(19) United States
(12) Patent Application Publication
(10) Pub. No.: US 2003/0152637 A1
(43) Pub. Date: Aug. 14, 2003

(54) Local anesthetic, and method of use

(76) Inventors: Mark Chasin, Manalapan, NJ (US); Glenn Van Buskirk, Basking Ridge, NJ (US); Richard Maskiewicz, Ridgefield, CT (US); Amol Ketkar, Audobon, PA (US); Kevin Burton, Fishkill, NY (US); Mohammed Shameem, Nanuet, NY (US); Craig Landau, Norwalk, CT (US); Celia Coles, Easton, CT (US); Ruth Swanton, New Haven, CT (US); Peter Lacouture, Newton, CT (US)

Correspondence Address: DAVIDSON, DAVIDSON & KAPPEL, LLC, 485 SEVENTH AVENUE, 14TH FLOOR, NEW YORK, NY, 10018, US

(21) Appl. No.: 10/057,301

(22) Filed: Jan. 25, 2002

Related U.S. Application Data
(60) Provisional application No. 60/264,186, filed on Jan. 25, 2001.

(51) Int. Cl. A61K 9/50

(52) U.S. Cl. 424/501

(57) ABSTRACT

This invention relates to pharmaceutical formulations administered via parenteral methods, which provide a prolonged localized analgesic effect. More particularly, the present invention concerns a pharmaceutically acceptable biocompatible biodegradable carrier containing a local anesthetic and the parenteral administration of such carrier in a manner such that a localized analgesic effect is attained for a prolonged period of time.

This application claims benefit of U.S. Provisional Application Serial No. 60/264,186, filed Jan. 25, 2001, the disclosure of which is hereby incorporated by reference in its entirety.

### TECHNICAL FIELD

This invention relates to pharmaceutical formulations administered via parenteral methods, which provide a prolonged localized analgesic effect. More particularly, the present invention concerns a pharmaceutically acceptable biocompatible biodegradable carrier containing a local anesthetic and the parenteral administration of such carrier in a manner such that a localized analgesic effect is attained for a prolonged period of time.

### BACKGROUND OF THE INVENTION

Local anesthetics are drugs, which provide local numbness and/or analgesia. While compounds utilized as general anesthetics reduce pain by producing a loss of consciousness, local anesthetics act by producing a loss of sensation in the localized area of administration in the body. The local anesthetics are a family of drugs with a long history of providing local anesthesia for surgery and painful procedures. In general, these products have a rapid onset, but a relatively short duration of action.

Different devices and formulations are known in the art for administration of local anesthetics. For example, local anesthetics can be delivered in solution or suspension by means of injection, infusion, infiltration, irrigation, topically and the like. Injection or infusion can be carried out acutely, or if prolonged local effects are desired, localized anesthetic agents can be administered continuously by means of a gravity drip or infusion pump.

Local anesthetics are disclosed in the following U.S. Pat Nos. 5,618,563; 5,747,060; 5,700,485; 5,942,241; 5,922,340; 6,046,187.

A relatively long-acting local anesthetic, bupivacaine hydrochloride, is commercially available as Marcaine® Hydrochloride and Sensorcaine, among others, in sterile isotonic solutions with and without epinephrine (as bitartrate) 1:200,000 for injection via local infiltration, peripheral nerve block, and caudal and lumbar epidural blocks. After injection of Marcaine® for caudal, epidural or peripheral nerve block in man, peak concentrations of bupivacaine in the blood are reached in 30 to 45 minutes, followed by a decline to insignificant levels during the next three to six hours.

A delivery system and method for local anesthetics which provides an extended period of local anesthesia, pain relief or analgesia is desireable. In particular, a delivery system and method, which is capable of being administered or injection resulting in a prolonged analgesic/anesthetic action is considered highly desirable.

### OBJECTS AND SUMMARY OF THE INVENTION

It is an object of the present invention to provide a formulation of a local anesthetic which is administrable, e.g., via injection, infiltration, or implantation to provide prolonged, local numbness, pain relief, local analgesia, local anesthesia or nerve blockage, at the site of administration.

It is another object of another preferred embodiment of the present invention to provide a biocompatible, biodegradable controlled release formulation of a local anesthetic which will provide an adequate (e.g., partial or full) sensory block (e.g., local analgesia, local anesthesia, or both) when administered at a desired site in a human patient, with a desired onset and prolonged duration of analgesic activity after administration, and which does so in a safe manner.

In accordance with the above objects and others, the present invention is directed in part to a controlled release formulation and method for providing local analgesia in a human, comprising administering at a desired site in a human patient a biocompatible, biodegradable controlled release carrier including a local anesthetic, the formulation providing an onset of local anesthesia or pain relief (local analgesia), local numbness or nerve blockade at the site of administration in a human which, upon first administration, occurs less than about 2 hours after administration, and a duration of effect which lasts for at least about 1 day after administration.

In certain embodiments, the invention is directed to a method for providing local analgesia, local anesthesia or nerve blockade in a human, comprising administering at a site in a human a formulation comprising a plurality of controlled release microspheres comprising bupivacaine free base and a biocompatible, biodegradable polymer comprising a 65:35 DL copolymer of lactic and glycolic acid having free carboxylic acid end groups, said copolymer having a molecular weight of about 40 kDa to about 120 kDa, said microspheres comprising from about 60% to about 85% bupivacaine free base, by weight, said microspheres being contained in a pharmaceutically acceptable medium for parenteral administration, said formulation having a concentration of bupivacaine free base from about 2.25 mg/ml to about 36.0 mg/ml and the formulation including a total amount of bupivacaine free base from about 45 mg to about 360 mg prior to administration, such that said formulation provides local analgesia, local anesthesia or nerve blockade at the site of administration less than about 2 hours after first administration, and a duration of local analgesia, local anesthesia or nerve blockade which lasts for at least about 1 day after first administration. The present invention is also directed to formulations utilized in this method.

In certain preferred embodiments, the duration of local analgesia is at least about 2 days, optionally the duration can be from about 2 to about 7 days after administration. In certain other preferred embodiments, the duration of local analgesia is from about 2 to about 4 days, or from about 3 to about 5 days, or from about 4 to about 7 days after administration.

In certain embodiments, the formulation further comprises a dose of a second local anesthetic in immediate release form, said second local anesthetic providing an onset of activity in less than about 5 minutes after administration of the formulation.

In additional embodiments, the formulation comprises a plurality of controlled release microspheres containing the local anesthetic. In certain preferred embodiments, the formulation further comprises an augmenting agent in an amount effective to prolong the effect of the local anesthetic.

In certain preferred embodiments, the local anesthetic incorporated into the formulation is bupivacaine free base.

The invention is further related to a formulation for providing local anesthesia or local analgesia or pain relief or nerve blockage at a site in a patient, comprising a plurality of biocompatible, biodegradable controlled release microspheres containing a dose of local anesthetic, providing an onset of local analgesia at the site of administration which occurs less than about 2 hours after administration, and a duration of local analgesia which lasts for at least about 1 day after administration. The microspheres may be suspended in a pharmaceutically acceptable medium for parenteral injection or infiltration prior to administration at the desired site.

In certain preferred embodiments, the microspheres further comprise an augmenting agent and provide local analgesia which lasts for at least 72 hours after administration.

In certain preferred embodiments, the microspheres further comprise an augmenting agent and provide local analgesia which lasts for at least about 4 days after administration.

In certain preferred embodiments, the formulation provides a measurable change in sensory responses at the site of administration in a human patient for a time period from about 2 days to about 7 days after administration.

In certain preferred embodiments, the local anesthetic formulations of the invention include an augmenting agent and provide a measurable change in sensory responses at the site of administration in a human patient for a time period from about 4 days to about 7 days after administration.

In other preferred embodiments, the formulations do not include an effective amount of an augmenting agent and provide a measurable change in sensory responses at the site of administration in a human patient for a time period from about 1 day to about 3 days after administration. Opionally the formulations contain no augmenting agent.

In certain preferred embodiments, the local anesthetic formulation further comprises a second local anesthetic in immediate release form, said formulation providing an onset of activity not more than 5 minutes after parenteral administration of the formulation.

The invention is further related to methods of treatment, comprising administering an effective amount of the formulations comprising a biocompatible, biodegradable controlled release carrier such as those described herein containing the local anesthetic (with or without optional augmenting agent) to a human patient or to a mammal.

The controlled release local anesthetic dosage form may be injected, infiltrated, implanted or administered in any other fashion known to those skilled in the art, at the site where the anesthetic is to be released. This can be prior to surgery, at the time of surgery, or following removal (discontinuation) or reversal of a systemic anesthetic or trauma or injury.

In certain preferred embodiments, the local anesthetic is incorporated into a biocompatible, biodegradable polymer, preferably in the form of microspheres or microcapsules, which are in turn suspended in a pharmaceutically acceptable medium for administration (e.g., injection, trocar, or other means of infiltration) a desired site in the patient (e.g., subcutaneously). The local anesthetic loaded microspheres may be extended duration local anesthetic formulations (“EDLA”) which extend the duration of the analgesia to, e.g., about 4 to about 5 days after administration. The prolonged duration of EDLA formulations may be made possible via the incorporation of an augmenting agent (e.g., a glucocorticosteroid such as dexamethasone). In other preferred embodiments, the local anesthetic loaded microspheres do not incorporate an augmenting agent, and the duration of analgesia lasts for about 1 to about 3 days after administration. Such formulations are referred to herein as an intermediate duration local anesthetic (“IDLA”). In preferred embodiments, the onset of measurable changes in sensory findings at the site of administration (indicative of analgesia) occur within about 2 hours with either the EDLA or the IDLA formulations.

In certain preferred embodiments, the formulations of the present invention comprise microcapsules in which the local anesthetic (e.g., bupivacaine base) with or without optional augmenting agent (e.g.,dexamethasone) is not uniformly distributed throughout the controlled release carrier (e.g., PLGA). In certain preferred embodiments, the microcapsules comprise a “shell” and a “core”, the bulk of the drug(s) being found in the core (e.g., about 60-100%, preferably about 70-90%), and the remainder of the drug(s) is found in the shell of the microcapsules. In further preferred embodiments, such microcapsules have a mean particular size preferably smaller than 200 microns, and preferably have a particular size distribution from about 5 to about 150 microns, more preferably from about 25 to about 125 microns. In further preferred embodiments, the “shell” of the microcapsule is from about 1 to about 10 microns in mean thickness, and more preferably to about 3 to about 5 microns in mean thickness.

In certain embodiments, the invention is further directed to the disclosed formulations and methods which exhibit particular pharmacokinetic parameters as disclosed herein which can be measured by microdialysis.

As used herein, the terms “local anesthetic agent” or “local anesthetic” means any drug, which provides local numbness, pain relief, nerve blockage, analgesia, and/or anesthesia. The term also includes, but is not limited to, any drug which, when locally administered, e.g., topically or by infiltration or injection, provides localized full or partial inhibition of sensory perception and/or motor function. Under either definition, the localized condition so induced is also referred to herein as “local analgesia”. For purposes of the present invention, the phrase “local anesthetic” also includes, but is not limited to, drugs which, when locally administered, e.g., topically or by infiltration or injection, provide localized full or partial inhibition of sensory perception and/or motor function. Commonly known local anesthetic agents include bupivacaine, levo-bupivacaine, ropivacaine, benzocaine, dibucaine, procaine, chloroprocaine, prilocaine, mepivacaine, etidocaine, tetracaine, lidocaine, and xylocaine, as well as anesthetically active derivatives, analogs and mixtures thereof. The phrase “local anesthetic agents” also can include those agents which are typically administered systemically, but which can be administered in a manner that results only in a local effect. The phrase “local anesthetic” also can include drugs of a different class than those traditionally associated with local anesthetic properties, such as morphine, fentanyl, and agents which, for example, can provide regional blockade of nociceptive pathways (afferent and/or efferent). Local anesthetics can be in the form of a salt, for example, the hydrochloride, bromide, acetate, citrate, carbonate or sulfate, or in the form of a free base. The free base generally provides a slower initial release and avoids an early “dumping” of the local anesthetic at the injection site.

The controlled release formulations and methods of the invention may be used in conjunction with any system for application, infiltration, implantation, insertion, or injection known in the art, including but not limited to microparticles, e.g., microspheres or microcapsules, liposomes, gels, pastes, trochars, tablets, implantable rods, pellets, plates or fibers and the like. The term “microspheres” as used herein is deemed to encompass matrices in which the drug (e.g., local anesthetic) is distributed (either uniformly or non-uniformly) throughout the biocompatible, biodegradable polymer. Microspheres in which the drug(s) is not uniformly distributed throughout the polymer are alternatively referred to herein as microcapsules. The term “microparticles” is interchangeably used herein with the term microspheres. In certain preferred embodiments, the local anesthetic microspheres are microcapsules.

As used herein, the terms controlled release and sustained release indicate a prolongation of the duration of release and/or duration of action of an active agent and are well understood in the art and are intended to be interchangeable, unless otherwise indicated.

As used herein, the term “patient” broadly refers to any animal, preferably a human, that is to be treated with the compositions and by the methods herein disclosed. The disclosed extended duration microparticle formulations can provide prolonged and effective administration of active agents. In particular, the disclosed methods and compositions will find use in veterinary practice and animal husbandry for, e.g., birds and mammals, wherever prolonged local anesthesia is convenient or desirable. In certain embodiments, the formulations are preferably used for companion animals such as dogs or cats, and additionally may be used in horses. In a preferred embodiment, the term “patient” includes humans in need of or desiring prolonged local analgesia or local nerve blockade or local numbness.

As used herein, the term “unit dose” refers to physically discrete units suitable as unitary dosages for mammalian subjects, each unit containing as the active ingredient a predetermined quantity of the local anesthetic. Examples of suitable unit doses of local anesthetic in accordance with the invention include liquid preparations in suitable containers for injection, sterile dry preparations for the extemporaneous preparation of sterile injectable preparations in a suitable liquid vehicle, or for administration as a solid implant.

The term “Cₘₐₓ ” as it is used herein is the highest plasma or tissue concentration of the drug attained after a single administration.

The term “Tₘₐₓ ” as it is used herein is the time period which elapses after administration of the dosage form until the plasma or tissue concentration of the drug attains the highest concentration after a single administration.

The term “AUC” as it is used herein is the area under the plasma or tissue concentration-time curve. The AUCt is the area under the curve for the measured interval and the term AUC ∞ is the extrapolated area under the curve.

The term “mean” for purposes of the present invention, when used to define a pharmacokinetic value represents the arithmetic mean value measured across a human population, e.g., as tested in the appended examples or larger.

### BRIEF DESCRIPTION OF THE FIGURES

FIG. 1 is a graph depicting the in vitro release of various Examples;

FIG. 2 is a graph showing in vivo efficacy (mean latency and percent responders assessed in the rat using a hotplate model) for various Examples;

FIG. 3 is a graph showing an average release profile for Example 2b;

FIG. A1 is a graph of the mean mechanical pain detection thresholds over time observed after administration of 40K EDLA and 120 K EDLA;

FIG. A2 is a graph of the mean mechanical pain detection thresholds over time for 1.25% 40K EDLA and 1.25% 40K IDLA;

FIG. A3 is a graph of the mean suprathreshold pain response-mechanical (VRS) scores over time observed after administration of 40K EDLA and 120K EDLA;

FIG. A4 is a graph of the mean suprathreshold pain response-mechanical (VRS) scores over time for 1.25% 40K EDLA and 1.25% 40K IDLA;

FIG. A5 is a graph of the mean mechanical touch detection thresholds over time observed after administration of 40K EDLA and 120K EDLA;

FIG. A6 is a graph of the mechanical touch detection thresholds over time for 1.25% 40K EDLA and 1.25% 40K IDLA;

FIG. A7 is a graph of the mean suprathreshold pain response-heat testing (VRS scores) over time for 40K EDLA and 120K EDLA;

FIG. A8 is a graph of the mean suprathreshold pain response-heat testing (VRS scores) over time for 1.25% 40K EDLA and 1.25% 40K IDLA;

FIG. A9 is a graph of the mean heat pain detection thresholds over time for 40K EDLA and 120K EDLA;

FIG. A10 is a graph of the mean heat pain detection thresholds over time for 1.25% 40K EDLA and 1.25% 40K IDLA;

FIG. A11 is a graph of the mean warm detection thresholds over time for 40K EDLA and 120K EDLA;

FIG. A12 is a graph of the mean warm detection thresholds over time for 1.25% 40K EDLA and 1.25% 40K IDLA;

FIG. A13 is a graph of the mean cool detection thresholds over time observed after administration of 40K EDLA and 120K EDLA;

FIG. C1 is a graph of the mean response to pin-prick over time observed after administration of 120K EDLA;

FIG. C2 is a graph of the mean response to pin-prick over time observed after administration of 40K EDLA;

FIG. C3 is a graph of the mean response to pin-prick over time for 2.5% 40K EDLA and 2.5% 40K IDLA;

FIG. C4 is a graph of the mean response to pin-prick over time for 1.25% 120K EDLA and 120K IDLA;

FIG. C5 is a graph of the mean response to pin-prick over time for 5.0% 40K EDLA;

FIG. C6 is a graph of the mean response to somesthetic testing over time for 2.5% 40K EDLA and 2.5% 40K IDLA;

FIG. C7 is a graph of the mean degree of numbness over time observed after administration of 120K EDLA;

FIG. C8 is a graph of the mean degree of numbness over time observed after administration of 40K EDLA;

FIG. C9 is a graph of the mean degree of numbness over time for 2.5% 40K EDLA and 2.5% 40K IDLA;

FIG. C10 is a graph of the mean degree of numbness over time for 5.0% 40K EDLA;

FIG. C11 is a graph of the mean plasma bupivacaine concentrations over time for 120K EDLA;

FIG. C12 is a graph of the mean plasma bupivacaine concentrations over time for 40K EDLA;

FIG. C13 is a graph of the mean plasma bupivacaine concentrations over time for 2.5% 40K EDLA and 2.5% 40K IDLA;

FIG. C14 is a graph of the mean plasma bupivacaine concentrations over time for 1.25% 120K EDLA and 1.25% 120K IDLA;

FIG. C15 is a graph of the mean plasma bupivacaine concentrations over time for 5.0% 40K EDLA ;

FIG. D1 shows the assessment areas on the back of the hand that were used for pinprick testing;

FIG. D2 shows the degree of analgesia/anesthesia experienced by subjects treated with 2.5% 120K EDLA, and the plasma bupivacaine concentrations, over time after administration;

FIG. D3 shows the degree of analgesia/anesthesia experienced by subjects treated with aqueous bupivacaine (0.5% AB-D), and the plasma bupivacaine concentrations, over time after administration;

FIG. E1 shows mean pinprick scores for 120K EDLA or aqueous bupivacaine over time, up to 50 days;

FIG. F1 shows the percent of subjects experiencing analgesia/anesthesia when treated with 40K EDLA or aqueous bupivacaine;

FIG. F2 shows the mean and range of duration of analgesia/anesthesia experienced by subjects treated with 40K EDLA or aqueous bupivacaine;

FIG. F3 is a graph of analgesia/anesthesia over time experienced by subjects treated with 1.25% 40K EDLA or 1.25% 40K IDLA;

FIG. F4 shows the percent of subjects experiencing temperature perception block over time when treated with 1.25% 40K EDLA or aqueous bupivacaine;

FIG. F5 is a graph of the mean and range of duration of temperature perception block over time experienced by subjects treated with 40K EDLA or aqueous bupivacaine;

FIG. F6 is a graph of the numbness scores over time experienced by subjects treated with 1.25% 40K EDLA and 1.25% 40K IDLA;

FIG. F7 is a graph of the peak mechanical touch detection thresholds over time experienced by subjects treated with 1.25% 40K EDLA or 1.25% 40K IDLA;

FIG. F8 is a graph of the mean plasma bupivacaine concentrations over time in subjects treated with 1.25% 40K EDLA and 1.25% 40K IDLA;

FIG. G1 is a graph of the degree of analgesia/anesthesia experienced by subjects treated with 40K EDLA and 120K EDLA;

FIG. G2 is a graph of the onset of analgesia/anesthesia experienced by subjects treated with 40K EDLA and 120K EDLA;

FIG. G3 is a graph of the mean level of analgesia/anesthesia experienced by subjects treated with 1.25% 40K and 1.25% 40K IDLA;

FIG. G4 is a graph of the mean level of temperature perception block experienced by subjects treated with 40K EDLA and 120K EDLA;

FIG. G5 is a graph of the temperature perception block experienced by subjects treated with 1.25% 40K EDLA and 1.25% 40K IDLA;

FIG. G6 is a graph of the degree of numbness experienced by subjects treated with 40K EDLA and 120K EDLA;

FIG. G7 is a graph of the degree of numbness experienced by subjects treated with 1.25% 40K EDLA and 1.25% 40K IDLA;

FIG. H1 is a histogram of the time to first pain >3 experienced by podiatric surgery patients treated with 40K EDLA or placebo;

FIG. H2 is a histogram of the time to first use of rescue medication by podiatric surgery patients treated with 40K EDLA or placebo.

FIG. J1 depicts a summary of study design of part I and part II of the microdialysis study.

FIG. J2 depicts the injection points made into an area of subcutaneous tissue in the microdialysis study.

FIG. J3 depicts the disposition of subjects in the microdialysis study.

### DETAILED DESCRIPTION

The formulations of the invention may be administered parenterally. Suitable locations for administration include but are not limited to, subcutaneous, intramuscular, intercostal, at a single nerve, epidural, or intra-articular. It is an object of another preferred embodiment of the invention to provide local analgesia or anesthesia to the following areas of the body: Superficial and/or Deep cervical plexus block in the neck, the Brachial Plexus by interscalene, supraclavicular, infraclavicular, and axillary approaches, the musculocutaneous nerve in the upper extremity, nerves in the elbow region (ulnar nerve, median nerve, radial nerve, lateral antebrachial cutaneous nerve); the nerves in the wrist area (ulnar, median, radial); the Lumbosacral Plexus (Psosas compartment, Lumbar plexus, Sciatic nerves: common peroneal nerve, superficial and deep peroneal nerves, anterior tibial nerve, sural nerve, anterior tibial nerve, musculocutaneous nerve, Tibial nerve); Knee joint nerves (common peroneal, tibial, saphenous); Lumbar Epidural, Cervical, Thoracic and Lumbar Spinal nerve roots, Intercostal nerves, Thoracic Spinal nerves, Spinal Accessory nerve, hypoglossal nerve; lateral femoral cutaneous nerve, suprascapular nerve femoral nerve, Obturator nerve, sacral nerves; Paracervical and Pudendal blocks in Obstetrics. Additionally, the formulations of the invention may be used with respect to the following nerves, which are susceptible to blockade in the area of pain therapy: specifically Sympathetic blockade: Stellate ganglion, Celiac plexus, Lumbar sympathetic, splanchnic nerves, vagus nerve; the head area, including: the Gasserian ganglion, sphenopalatine ganglion, posterior superior alveolar nerve, infraorbital and anterior superior alveolar nerves, inferior alveolar nerve, lingual nerve, superior laryngeal nerve, inferior or recurrent laryngeal nerve, branches of the ophthalmic nerve (lacrimal, frontal, and nasociliary), mandibular nerve, ethmoidal nerve, mental nerve, lingual nerve, facial nerve, glossopharyngeal nerve, the supraorbital and supratrochlear nerves; the maxillary nerve and palatine nerves; infraorbital, mental, occipital nerves, myofascial trigger points and Intercostal block (blockade of the thoracic spinal roots, dorsal branch, ventral branch at angle of rib, ventral branch in posterior axillary line; dorsolateral intercostal block); Inguinal and Iliohypogastric nerves; cervical plexus; phrenic nerve; peridural block (segmental, continuous epidural block, caudal and subarachnoid block, and neuraxially, as well as any other location at which the formulations of the invention would be considered useful.

In certain preferred embodiments, the formulations and methods of the invention may be further characterized by providing an in-vitro dissolution of the local anesthetic from the biocompatible biodegradable carrier as follows:

| TIME (Hours) | Percent Release |
|---|---|
| 0 | 0 |
| 0.25 | about 2 to about 32 |
| 0.5 | about 3 to about 60 |
| 1 | about 6 to about 86 |
| 1.5 | about 9 to about 92 |
| 2 | about 12 to about 94 |
| 3 | about 17 to about 97 |
| 4 | about 23 to about 97 |

The in-vitro dissolution range described above may be determined by subjecting the local anesthetic formulation to in-vitro conditions specified by the USP II Paddle Method, 100 RPM, 37 degrees Celcius, pH 3.0 in 900 ml of 10 mM sodium phosphate buffer.

In certain preferred embodiments, the dissolution ranges (determined as set forth above) are as follows:

| TIME (Hours) | Percent Release |
|---|---|
| 0 | 0 |
| 1 | From about 13 to about 36 |
| 2 | From about 33 to about 65 |
| 4 | From about 53 to about 87 |
| 8 | From about 72 to about 95 |
| 12 | From about 81 to about 98 |
| 18 | From about 89 to about 100 |
| 24 | From about 94 to about 100 |

The in-vivo efficacy of the formulations and methods of the invention may be further assessed in the rat using hotplate model, e.g., according to the procedure described in detail in IACUC No 9511-2199. The efficacy criteria established for formulations of the invention are mean latency greater than about 2 seconds, with a 12 second cut-off (this cutoff is imposed to prevent any possible damage to the animal). Latencies at 2 seconds are demonstrative of a statistically significant effect of the local anesthetic. Preferably, the mean latency under the rat hotplate model is greater than 7 seconds. Preferably, the percent responders is 50% or greater. Preferably, the formulations of the invention provide a mean latency under the rat hotplate model greater than about 7 seconds to about 12 seconds, with the percent of rats exhibiting the effect being at least about 50% of those tested.

Sensory testing in human models is useful in testing of local anesthetic formulations. In the appended examples, the local anesthetic activity in accordance with the invention was examined with reference to onset, peak density and duration of effect using seven specific modalities: 1) mechanical sensory testing (mechanical pain detection threshold using von Frey hairs; 2) suprathreshold (mechanical) testing using a single von Frey hair; 3) thermal sensory testing (warm detection threshold); 4) heat pain detection threshold; 5) suprathreshold (heat) testing; 6) cool detection threshold; and 7) tactile sensory testing (mechanical touch detection threshold). The varying degrees or levels of the results are indicative of the patient experiencing local pain relief, local numbness, and or local nerve blockade. The anesthetic activity of the formulations and methods of the invention was further characterized with respect to safety, by various measures of activity such as systemic blood plasma levels attained after administration at the localized site.

The formulations of the present invention preferably provide an onset of effect in humans at the site of administration, which occurs less than about 2 hours after administration, and a duration of local analgesia which lasts for at least about 1 to about 7 days after administration. The duration of effect is at least 1 day, but may be at least 2 days, at least 3 days, at least 4 days, at least 5 days, at least 6 days, at least 7 days, or more.

In certain preferred embodiments the formulations further comprise an augmenting agent in an amount effective to prolong the effect of the local anesthetic. In such embodiments, the formulations have a duration of local analgesia which lasts for at least about 4 days after administration, and in certain cases preferably for about 4 to about 7 days after administration. Such formulations are exemplified in the appended Examples, particularly via the formulation of Example 2. In certain other preferred embodiments, the duration of local analgesia is shorter, e.g., lasting until from about 24 to about 36 hours after administration. Such formulations are exemplified in the appended Examples, particularly via the formulation of Example 1.

However, as will be explained below, it is readily apparent to one skilled in the art that the exemplified formulations can be modified without altering the resultant duration of analgesia or anesthesia.

Formulations

Any pharmaceutically acceptable vehicle or formulation suitable for local infiltration or injection into a site to be anesthetized, that is able to provide a sustained release of an active agent may be employed to provide for prolonged local anesthesia and/or analgesia as needed. Slow release formulations known in the art include specially coated pellets, polymer formulations or matrices for surgical insertion or as sustained release microparticles, e.g., Microspheres or microcapsules, for implantation, insertion, infusion or injection, wherein the slow release of the active medicament is brought about through sustained or controlled diffusion out of the matrix and/or selective breakdown of the coating of the preparation or selective breakdown of a polymer matrix. Other formulations or vehicles for sustained or immediate delivery of an agent to a preferred localized site in a patient include, e.g., suspensions, emulsions, gels, liposomes and any other suitable art known delivery vehicle or formulation acceptable for subcutaneous or intramuscular administration.

A wide variety of biocompatible materials may be utilized as a controlled release carrier to provide the controlled release of the local anesthetic. Any pharmaceutically acceptable biocompatible polymer known to those skilled in the art may be utilized. It is preferred that the biocompatible controlled release material degrade in vivo within about one year, preferably within about 3 months, more preferably within about two months. More preferably, the controlled release material will degrade significantly within one to three months, with at least 50% of the material degrading into non-toxic residues, which are removed by the body, and 100% of the drug being released within a time period within about two weeks, preferably within about 2 days to about 7 days. A degradable controlled release material should preferably degrade by hydrolysis, either by surface erosion or bulk erosion, so that release is not only sustained but also provides desirable release rates. However, the pharmacokinetic release profile of these formulations may be first order, zero order, bi- or multi-phasic, to provide the desired reversible local anesthetic effect over the desired time period.

Suitable biocompatible polymers can be utilized as the controlled release material. The polymeric material may comprise biocompatible, biodegradable polymers, and in certain preferred embodiments is preferably a copolymer of lactic and glycolic acid. Preferred controlled release materials which are useful in the formulations of the invention include the polyanhydrides, polyesters, co-polymers of lactic acid and glycolic acid (preferably wherein the weight ratio of lactic acid to glycolic acid is no more than 4:1 i.e., 80% or less lactic acid to 20% or more glycolic acid by weight)) and polyorthoesters containing a catalyst or degradation enhancing compound, for example, containing at least 1% by weight anhydride catalyst such as maleic anhydride. Examples of polyesters include polylactic acid, polyglycolic acid and polylactic acid-polyglycolic acid copolymers. Other useful polymers include protein polymers such as collagen, gelatin, fibrin and fibrinogen and polysaccharides such as hyaluronic acid.

The polymeric material may be prepared by any method known to those skilled in the art. For example, where the polymeric material is comprised of a copolymer of lactic and glycolic acid, this copolymer may be prepared by the procedure set forth in U.S. Pat. No. 4,293,539 (Ludwig, et al.). Alternatively, copolymers of lactic and glycolic acid may be prepared by any other procedure known to those skilled in the art.

Various commercially available poly (lactide-co-glycolide) materials (PLGA) may be used in the preparation of the microspheres of the present invention. For example, poly(d,1-lactic-co-glycolic acid) is commercially available from Alkermes, Inc. (formerly Medisorb Technologies International L.P. (Cincinnati, Ohio.)). A preferred product commercially available from Medisorb is a 50:50 poly (D, L) lactic co-glycolic acid known as MEDISORB 5050 DL. This product has a mole percent composition of 50% lactide and 50% glycolide. Other suitable commercially available products are Medisorb 65:35 DL, 75:25 DL, 85:15 DL and poly(d,1-lactic acid) (d,1-PLA). Poly(lactide-co-glycolides) are also commercially available from Boerhinger Ingelheim (Germany) under its RESOMER(® mark, e.g., PLGA 50:50 (RESOMER RG 502), PLGA 75:25 (RESOMER RG 752) and d,1-PLA (RESOMER RG 206), and from Birmingham Polymers (Birmingham, Ala.). These copolymers are available in a wide range of molecular weights and ratios of lactic to glycolic acid.

Other useful polymers include polylactides, polyglycolides, polyanhydrides, polyorthoesters, polycaprolactones, polyphosphazenes, polyphosphoesters, polysaccharides, proteinaceous polymers, soluble derivatives of polysaccharides, soluble derivatives of proteinaceous polymers, polypeptides, polyesters, and polyorthoesters or mixtures or blends of any of these. Pharmaceutically acceptable polyanhydrides which are useful in the present invention have a water-labile anhydride linkage. The rate of drug release can be controlled by the particular polyanhydride polymer utilized and its molecular weight. The polysaccharides may be poly-1,4-glucans, e.g., starch glycogen, amylose, amylopectin, and mixtures thereof. The biodegradable hydrophilic or hydrophobic polymer may be a water-soluble derivative of a poly-1,4-glucan, including hydrolyzed amylopectin, hydroxyalkyl derivatives of hydrolyzed amylopectin such as hydroxyethyl starch (HES), hydroxyethyl amylose, dialdehyde starch, and the like. The polyanhydride polymer may be branched or linear. Examples of polymers which are useful in the present invention include (in addition to homopolymers and copolymers of poly(lactic acid) and/or poly(glycolic acid)) poly[bis(p-carboxyphenoxy) propane anhydride] (PCPP), poly[bis(p-carboxy)methane anhydride] (PCPM), polyanhydrides of oligomerized unsaturated aliphatic acids, polyanhydride polymers prepared from amino acids which are modified to include an additional carboxylic acid, aromatic polyanhydride compositions, and co-polymers of polyanhydrides with other substances, such as fatty acid terminated polyanhydrides, e.g., polyanhydrides polymerized from monomers of dimers and/or trimers of unsaturated fatty acids or unsaturated aliphatic acids. Polyanhydrides may be prepared in accordance with the methods set forth in U.S. Pat. No. 4,757,128, hereby incorporated by reference. Polyorthoester polymers may be prepared, e.g., as set forth in U.S. Pat. No. 4,070,347, hereby incorporated by reference. Polyphosphoesters may be prepared and used as set forth in U.S. Pat. Nos. 6,008,318, 6,153,212, 5,952,451, 6,051,576, 6,103,255, 5,176,907 and 5,194,581, all of which are hereby incorporated by reference herein in their entireties.

Proteinaceous polymers may also be used. Proteinaceous polymers and their soluble derivatives include gelation biodegradable synthetic polypeptides, elastin, alkylated collagen, alkylated elastin, and the like. Biodegradable synthetic polypeptides include poly-(N-hydroxyalkyl)-L-asparagine, poly-(N-hydroxyalkyl)-L-glutamine, copolymers of N-hydroxyalkyl-L-asparagine and N-hydroxyalkyl-L-glutamine with other amino acids. Suggested amino acids include L-alanine, L-lysine, L-phenylalanine, L-valine, L-tyrosine, and the like.

In additional embodiments, the controlled release material, which in effect acts as a carrier for the local anesthetic, can further include a bioadhesive polymer such as pectins (polygalacturonic acid), mucopolysaccharides (hyaluronic acid, mucin) or non-toxic lectins or the polymer itself may be bioadhesive, e.g., polyanhydride or polysaccharides such as chitosan.

In embodiments where the biodegradable polymer comprises a gel, one such useful polymer is a thermally gelling polymer, e.g., polyethylene oxide, polypropylene oxide (PEO-PPO) block copolymer such as Pluronic® F127 from BASF Wyandotte. In such cases, the local anesthetic formulation may be injected via syringe as a free-flowing liquid, which gels rapidly above 30° C. (e.g., when injected into a patient). The gel system then releases a steady dose of local anesthetic at the site of administration.

Microspheres

In certain embodiments of the invention, microspheres are manufactured using a method that evenly disperses the local anesthetic throughout the formulation, such as emulsion preparation, solvent casting, spray drying or hot melt, rather than a method such as compression molding. In certain preferred embodiments the microspheres are manufactured using a method that causes the local anesthetic to be concentrated toward the center of the microspheres, i.e., to form microcapsules. In certain embodiments it would be acceptable to have the local anesthetic concentrated toward the outside of the microspheres.

In certain preferred embodiments of the invention, the substrate comprises a plurality of microcapsules laden with the local anesthetic agent with or without an augmenting agent. Microcapsules may be prepared, for example, by dissolving or dispersing the local anesthetic agent in an organic solvent and dissolving a wall forming material (polystyrene, alkylcelluloses, polyesters, polysaccharides, polycarbonates, poly(meth)acrylic acid ester, cellulose acetate, hydroxypropylmethylcellulose phthalate, dibutylaminohydroxypropyl ether, polyvinyl butyral, polyvinyl formal, polyvinylacetal-diethylamino acetate, 2-methyl-5-vinyl pyridine methacrylate-methacrylic acid copolymer, polypropylene, vinylchloride-vinylacetate copolymer, glycerol distearate, etc.) in the solvent; then dispersing the solvent containing the local anesthetic agent and wall forming material in a continuous-phase processing medium, and then evaporating a portion of the solvent to obtain microcapsules containing the local anesthetic agent in suspension, and finally, extracting the remainder of the solvent from the microcapsules. This procedure is described in more detail in U.S. Pat. Nos. 4,389,330 and 4,530,840.

In the case of polymeric materials, biocompatibility may be enhanced by recrystallization of either the monomers forming the polymer and/or the polymer using standard techniques.

A desired release profile can be achieved by using a given polymer molecular weight and hydrophilicity, a mixture of polymers having different release rates, and/or different percent loading of local anesthetic and/or augmenting agent, for example, local anesthetic and or augmenting agent releasing in one day, three days, and one week. In addition, a mixture of microspheres having one or more different local anesthetic agents, having the same or different controlled release profile, can be utilized to provide the benefits of different potencies and spectrum of activity during the course of treatment.

The microspheres are preferably manufactured in a size distribution range suitable for local infiltration or injection. The diameter and shape of the microcapsules, microspheres or other particles can be manipulated to modify the release characteristics. For example, larger diameter microcapsules or microspheres will typically provide slower rates of release and reduced tissue penetration and smaller diameters of microcapsules or microspheres will produce the opposite effects, relative to microspheres of different mean diameter but of the same composition. The mean diameter of injectable microcapsules or microspheres is in a size range, for example, from about 5 microns to about 200 microns in diameter. In a more preferred embodiment, the microcapsules or microspheres range in mean diameter from about 20 to about 130 microns.

Other particle shapes which may be used to prepare the local anesthetic formulations of the invention, such as, for example, cylindrical shapes, can also modify release rates by virtue of the increased ratio of surface area to mass inherent to such alternative geometrical shapes, relative to a spherical shape.

The polymers used in certain preferred embodiments of the present invention, particularly poly(lactide co-glycolide) (referred to herein as “PLGA”), preferably have a molecular weight from about 5 kilodaltons (kDa) to about 200 kDa. Preferably the molecular weight is from about 20 kDa to about 50 kDa. The inherent viscosity of the preferred polymeric materials is from about 0.19 to about 0.7 dl/g, and most preferably from about 0.25 to about 0.43 dl/g. In certain preferred embodiments, these polymers are acid-terminated with carboxylic acid. In certain preferred embodiments, the polymer used in the microspheres is a poly(lactide co-glycolide) wherein the ratio of lactic acid to glycolic acid is from about 75:25 to about 50:50, preferably 65:35. In certain preferred embodiments, the polymer is a 65:35 DL copolymer of lactic and glycolic acid (inherent viscosity from about 0.25 to about 0.42 dL/g; molecular weight approximately 40 kDa with free carboxyl groups). In certain preferred embodiments, the local anesthetic incorporated in the polymer is bupivacaine base.

The local anesthetic is preferably incorporated into the microspheres in a percent loading between 0.1% and 90% or more, by weight, preferably between 5% and 80%, or more, by weight and more preferably between 65 and 80%, or more, by weight. In an even more preferred embodiment, the local anesthetic is loaded at about 70-75% by weight.

Diffusional release of the local anesthetic from the microspheres of the present invention can be altered in a number of ways including modification of polymer properties (molecular weight (MW), comonomer ratio and hydrophilicity), increasing matrix porosity via altering process parameters or through the addition of porosogens (inorganic salts and polyethylene glycol), and increasing dissolution rate/solubility of the drug.

Diffusivity of a Matrix

Diffusion through a sphere has been mathematically expressed through modification of Fick's First law as

$$\frac{ⅆ M}{ⅆ t} = \frac{4 \cdot D \cdot ϵ \cdot {C}_{s} \cdot R \cdot r \cdot π}{T \cdot h} \qquad ( 1 )$$

The flux (dM/dt) of a drug through the polymer matrix is dependent on diffusion coefficient (D), the porosity of the matrix (ε), the solubility of the drug in the release media (Cₛ), the radius of the matrix (R), the spherical boundary layer surface (r), the distance the drug must travel to reach the surface (h) and the tortuosity (T). As evidenced by Eq. 1, the options for changing the diffusional release without changing the properties of the drug can be controlled by increasing the porosity (decreasing tortuosity) of the matrix, changing the radius of the spheres (particle size), and decreasing the MW of the polymer (increasing D).

In certain preferred embodiments, the microspheres are porous microcapsules. In such circumstances, the diffusivity from the microcapsules may be better characterized by equation 2:

$$\frac{ⅆ M}{ⅆ t} = \frac{4 \cdot D \cdot ϵ \cdot {C}_{s} \cdot r \cdot π}{T \cdot h} \qquad ( 2 )$$

The flux (dM/dt) of a drug through the polymer matrix is dependent on diffusion coefficient (D), the porosity of the matrix (ε), the solubility of the drug in the release media (Cₛ), the spherical boundary layer surface (r), the distance the drug must travel to reach the surface (h) and the tortuosity (T). As evidenced by equation 2, the options for changing the diffusional release without changing the properties of the drug are limited to increasing the porosity (decreasing tortuosity) of the matrix, changing the thickness of the encapsulating polymer shell (decreasing h) and increasing the spherical surface area (r).

Change of Polymer Properties

Polymer properties such as molecular weight (MW), comonomer ratio and type of polymer end group can all play a role in determining the structure of the encapsulating shell and in drug diffusion through the shell. As hydration of the encapsulating shell matrix increases, so does the rate of diffusion through decreased tortuosity (diffusional resistance) in the swollen matrix and increased dissolution and transport.

Polymer MW can be used to manipulate the release profiles. In general, polymers with lower MW produce increased release due to formation of an encapsulating shell having greater porosity (decreased tortuosity) and increased flux.

Comonomer Ratio

Comonomer ratio is another important property of the polymer, which can be used to modify release patterns. Because lactic acid is more hydrophobic than glycolic acid, decreasing the lactic acid content can increase matrix hydrophilicity and increase hydration of the matrix (with concomitant tortuosity decrease). Modification of the comonomer ratio can significantly impact the efficacy of the dosage forms.

End Group

PLGAs are terminated with either an ester or a free carboxylic acid depending on the nature of the synthesis process. The carboxylic acid-terminated polymers are more hydrophilic in nature due to the ionizable functionality. These polymers hydrate more rapidly leading to more rapid degradation when compared to the less hydrophilic ester-terminated polymers. The more hydrophilic polymers also yield a more porous encapsulating shell. These effects are more prominent with the lower MW polymers as the contour length to end group ratio is smaller. In the higher MW polymers, changing the end groups has less effect as the physio-chemical properties of the polymer are dominated by the polymer backbone. Further, the rapid hydration of hydrophilic polymers should result in faster dissolution of bupivacaine and a faster release rate through the polymer shell matrix.

A related phenomenon that may increase the dissolution of the drug is the microenvironmental effect. This refers to the possibility of a lowered pH environment in the microspheres when using the lower MW hydrophilic PLGA. The lowered pH results from ionization of carboxylic acid residues initially present. Such a localized acidic environment may aid in dissolution of bupivacaine base and thereby increase its release rate.

Polymer Blends

Polymer blending offers another potential possibility for altering release. Polymer blending will modify the release profile while keeping the drug encapsulated.

Porosogens

Another possibility in increasing diffusion through the encapsulating shell matrix is to increase porosity. Porosogens can be added to the formulation to facilitate pore formation. A variety of possibilities exist which include inorganic salts and water soluble polymers such as polyethylene glycol.

Inorganic Salts as Porosogens

Calcium chloride is soluble in ethyl acetate and therefore can be used directly in the organic phase without jeopardizing the inline sterile filtration. In addition to CaCl₂; NaCl, citrate and ascorbate can be used to increase porosity. Polyethylene glycol (PEG) is a water soluble polymer which can be used to induce porosity. PEGs are available in a wide range of MW ensuring versatility in their implementation. Useful PEGs include, e.g., PEGs of MW 8000 and 4600.

Other Techniques to Alter Release Rate

The salt form of local anesthetics (e.g., bupivacaine HCl) has a better aqueous solubility than the base (e.g., bupivacaine base). This tends to increase the dissolution rate of the encapsulated drug and thereby increase the release rate. The addition of bupivacaine HCl to bupivacaine base can also result in the drug substance being a porosogen.

Drug Load

To avoid a burst release, decreased duration of action or a toxicology concern, e.g., when shifting to a lower MW polymer, one may decrease the drug loading in the microspheres.

Rate of Solvent Extraction

The rate at which the solvent is removed from the microspheres may influence the morphology of the microspheres (see the method of manufacture set forth below). Removing the solvent at a rapid rate produces microspheres with a very porous internal structure while removing the solvent slowly results in an internal cavity devoid of polymer.

Methods of Manufacture of Microspheres

In certain preferred embodiments, the local anesthetic formulations are prepared during the manufacture of microcapsules containing the drug. The formulations may be prepared as a plurality of microcapsules laden with the local anesthetic agent with or without the augmenting agent.

In preferred embodiments of the invention, the local anesthetic microsphere formulations are prepared by (i) forming an “oil-in-water” emulsion from an aqueous solution containing a surfactant and/or thickening agent (process water) and an organic solvent (oil) containing bupivacaine base raw material and a biocompatible, bioerodable polymer; (ii) removing the solvent following emulsification, via the use of an aqueous quench, allowing the microcapsules laden with the local anesthetic to form and harden. In certain preferred embodiments, the aqueous phase is prepared by adding a suitable quantity of polyvinyl alcohol (PVA) to water, heating to dissolve the PVA, and thereafter adding a suitable quantity of ethyl acetate to form the process water (aqueous phase) of the emulsion. In certain preferred embodiments, the organic phase is prepared by dissolving the polymer in a suitable solvent and thereafter adding the bupivacaine base and mixing until dissolved.

In embodiments where an augmenting agent is included in the microcapsules, the augmenting agent can also be added to the organic phase before or after the addition of the local anesthetic. In certain preferred embodiments, the augmenting agent is dexamethasone, which is added to the organic solvent prior or subsequent to the addition of bupivacaine base.

Microcapsules may also be prepared, for example, by dissolving or dispersing the local anesthetic agent in an organic solvent and dissolving a wall forming material (polystyrene, alkylcelluloses, polyesters, polysaccharides, polycarbonates, poly(meth)acrylic acid ester, cellulose acetate, hydroxypropylmethylcellulose phthalate, dibutylaminohydroxypropyl ether, polyvinyl butyral, polyvinyl formal, polyvinylacetal-diethylamino acetate, 2-methyl-5-vinyl pyridine methacrylate-methacrylic acid copolymer, polypropylene, vinylchloride-vinylacetate copolymer, glycerol distearate, etc.) in the solvent; then dispersing the solvent containing the local anesthetic agent and wall forming material in a continuous-phase processing medium, and then evaporating or extracting a portion of the solvent to obtain microcapsules containing the local anesthetic agent as an encapsulated suspension, and finally, extracting the remainder of the solvent from the microcapsules. This procedure is described in more detail in U.S. Pat. Nos. 4,389,330 and 4,530,840.

Methods for manufacture of microcapsules and microspheres are well known and are typified in the appended examples. Examples of suitable methods of making microcapsules and/or microspheres include solvent extraction, solvent evaporation, phase separation and fluidized bed coating.

In solvent extraction/evaporation procedures, the local anesthetic agent, if soluble in organic solvents, may be entrapped in the biodegradable polymer by dissolving the polymer in a volatile or water soluble organic solvent, adding the drug to the organic phase, emulsifying the organic phase in water which contains less than 2% polyvinyl alcohol, and finally removing the solvent under vacuum, or by addition to a large excess of water, to form discrete, hardened monolithic microspheres.

Phase separation microencapsulation procedures are suitable for entrapping water-soluble agents in the polymer to prepare microcapsules and microspheres. Phase separation involves coacervation of the polymer from an organic solvent by addition of a nonsolvent such as silicone oil. Microcapsules/microspheres may be prepared by the process of Ramstack et al., as described in WO 95/13799, the disclosure of which is incorporated herein in its entirety. The Ramstack et al. process essentially provides for a first phase, including an active agent and a polymer, and a second phase, that are pumped through a static mixer into a quench liquid to form microparticles containing the active agent. The first and second phases can optionally be substantially immiscible and the second phase is preferably free from solvents for the polymer and the active agent and includes an aqueous solution of an emulsifier.

In fluidized bed coating, the drug is dissolved in an organic solvent along with the polymer. The solution is then processed, e.g., through a Wurster air suspension coating apparatus to form the final microcapsule product.

Implants

The biodegradable sustained release materials may be used to prepare controlled release local anesthetic implants. The implants may be manufactured, e.g., by compression molding, injection molding, and screw extrusion, whereby the local anesthetic agent is loaded into the polymer. Implantable fibers can be manufactured, e.g., by blending the local anesthetic agent with the sustained release material and then extruding the mixture, e.g., under pressure, to thereby obtain biodegradable fibers. In certain preferred embodiments, the augmenting agent may be incorporated into the implant, or may be coated onto a surface of the implant.

Pellets, slabs or solid formulations shaped to fit particular locations, e.g., articular joints, may be surgically placed into a site where release of anesthetic agent is desired. Sustained release gels, pastes or suspensions, including gels, pastes or suspension containing microparticles, may also be administered to obtain localized anesthesia. For treatment of joint pain of the back or neck, the dosage form may be administered by intra-articular injection into one or more facet joints.

Other Formulations

Certain formulations may comprise a non-polymeric composition for in situ formation of a solid matrix in a human or an animal, for example the formulations described in U.S. Pat. Nos. 6,120,789 and 5,990,194. Such compositions are composed of a biocompatible, non-polymeric material and a pharmaceutically-acceptable, organic solvent, and are biodegradable and/or bioerodible, and substantially insoluble in aqueous or body fluids. The organic solvent component solubilizes the non-polymeric material, and has a solubility in water or other aqueous media ranging from miscible to dispersible. When placed into an implant site in an animal or a human, the non-polymeric composition eventually transforms into a solid structure.

In certain other formulations, such as those described in U. S. Pat. No. 5,747,058, a composition for the controlled release of substances is provided that includes: (i) a non-polymeric, non-water soluble high-viscosity liquid carrier material (HVLCM) of viscosity of at least 5,000 cP at 37.degree. C. that does not crystallize neat under ambient or physiological conditions; and (ii) a substance to be delivered. The HVLCM may be mixed with a viscosity lowering water soluble or miscible solvent such as ethanol, dimethylsulfoxide, ethyl lactate, ethyl acetate, benzyl alcohol, triacetin, N-methylpyrrolidone, propylene carbonate, glycofurol, freons such as trichlorofluoromethane and dichlorofluoromethane, dimethyl ether, propane, butane, dimethyl formamide, dimethyl acetamide, diethylene carbonate, butylene glycol, N-(beta-hydromethyl)lactamide, dioxolanes, and other amides, esters, ethers, alcohols, to form a lower viscosity liquid carrier material (LVLCM), which is mixed with the substance to be delivered, prior to administration. The LVLCM preferably has a viscosity less than 1000 cP, and more particularly less than 200 cP, and is useful for in vivo applications. On administration, the composition is placed into the body or on a surface, and the solvent dissipates or diffuses away from the LVLCM, forming in-situ a highly viscous implant or composition that releases the substance over time. By appropriate selection of the solvent and the HVLCM, a wide variety of pre- and post-administration composition viscosities can be achieved. The HVLCM as described herein is biodegradable. The HVLCM significantly decreases in viscosity when mixed with a solvent to form a LVLCM that can be mixed with a substrate for controlled delivery. The LVLCM/substrate composition is typically easier to place in the body than a HVLCM/substrate composition, because it flows more easily into and out of syringes or other implantation means, and can easily be formulated as an emulsion. In certain instances, sucrose acetate isobutyrate (“SAIB”), a sucrose molecule esterified with two acetic acid and six isobutyric acid moieties, is used as the HVLCM. SAIB is orally non-toxic and is currently used as to stabilize emulsions in the food industry. It is a very viscous liquid and has an unusual property that there is a dramatic change in viscosity with small additions of heat or with the addition of solvents. It is soluble in a large number of biocompatible solvents. When in solution or in an emulsion, SAIB can be applied via injection or an aerosol spray. SAIB is compatible with cellulose esters and other polymers that can affect the rate of delivery of the substance. In other instances, the HVLCM can be stearate esters such as those of propylene glycol, glyceryl, diethylaminoethyl, and glycol, stearate amides and other long-chain fatty acid amides, such as N,N′-ethylene distearamide, stearamide MEA and DEA, ethylene bistearamide, cocoamine oxide, long-chain fatty alcohols, such as cetyl alcohol and stearyl alcohol, long-chain esters such as myristyl myristate, beheny erucate, and glyceryl phosphates. In another instance, the HVLCM is acetylated sucrose distearate (Crodesta A-10). The HVLCM is present in the composition in any amount that achieves the desired affect. For example, as a tissue coating or for the prevention of adhesions, the HVLCM can be used alone as a protective film or bolus, or with a substrate that enhances the properties or effect of the material. The HVLCM is typically present in controlled delivery compositions in an amount in the range from about 99.5 percent to about 10 percent by weight, more typically, between 95 and 25 percent, and most typically, between 85 and 45, relative to the total weight of the composition.

In other embodiments of the invention, the controlled release material comprises an artificial lipid vesicle, or liposome. The use of liposomes as drug delivery systems is known, and comprehensive review articles on their properties and clinical applications are available; see, e.g., Barenholz and Amselem, in “Liposome Technology”, 2nd ed., G. Gregoriadis, ed., CRC Press, 1992; Lichtenberg and Barenholz, in Methods for Biochemical Analysis, 33, D. Glick, ed., 1988. A liposome is defined as a structure consisting of one or more concentric lipid bilayers separated by water or aqueous buffer compartments. These hollow structures, which have an internal aqueous compartment, can be prepared with diameters ranging from 20 nm to 10 μm. They are classified according to their final size and preparation method as: SUV, small unilamellar vesicles (20-50 mn); LUV, large unilamellar vesicles (100 nm); REV, reverse phase evaporation vesicles (0.5 μm); and MLV, large multilamellar vesicles (2-10 μm).

Liposomes as described herein will vary in size. Preferably, the liposomes have a diameter between 100 nm and 10 microns or greater. A wide variety of lipid materials may be used to form the liposomes including natural lecithins, e.g., those derived from egg and soya bean, and synthetic lecithins, the proviso being that it is preferred that the lipids are non-immunogenic and bio-degradable. Also, lipid-based materials formed in combination with polymers may be used, such as those described in U.S. Pat. No. 5,188,837 to Domb.

Examples of synthetic lecithins which may be used together with their respective phase transition temperatures, are di-(tetradecanoy)phosphatidylcholine (DTPC) (23° C.), di-(hexadecanoyl)phosphatidylcholine (DHPC) (41° C.) and di-(octandecanoyl) phosphatidylcholine (DOPC) (55° C.). Di-(hexadecanoyl) phosphatidycholine is preferred as the sole or major lecithin, optionally together with a minor proportion of the di-(octadecanoyl) or the di-(tetradecanoyl) compound. Other synthetic lecithins which may be used are unsaturated synthetic lecithins, for example, di-(oleyl)phosphatidyl-choline and di-(linoleyl)phosphatidylcholine. In addition to the main liposome-forming lipid or lipids, which are usually phospholipids, other lipids (e.g. in a proportion of 5-40% w/w of the total lipids) may be included, for example, cholesterol or cholesterol stearate, to modify the structure of the liposome membrane, rendering it more fluid or more rigid depending on the nature of the main liposome-forming lipid or lipids.

In certain embodiments, the augmenting agent is incorporated along with the local anesthetic agent into the lipid. In other preferred formulations, the lipids containing the local anesthetic agent are dispersed in a pharmaceutically acceptable aqueous medium. The augmenting agent may be incorporated into this aqueous medium. In a further embodiment, a portion of the dose of the local anesthetic is incorporated into the aqueous medium in immediate release form. The resultant formulation is an aqueous suspension which may comprise the local anesthetic and/or augmenting agent partitioned between a free aqueous phase and a liposome phase.

As an even further alternate embodiment, liposomes containing local anesthetic may be combined in an aqueous phase where liposomes containing the augmenting agent to form an aqueous pharmaceutical suspension useful for administration at the desired site in the patient to be anesthetized. This may be accomplished via injection or implantation. Liposomes may be prepared by dissolving an appropriate amount of a phospholipid or mixture or phospholipids together with any other desired lipid soluble components (e.g., cholesterol, cholesterol stearate) flowing in a suitable solvent (e.g., ethanol) and evaporating to dryness. An aqueous solution of the local anesthetic, optionally with augmenting agent, may then be added and mixed until a lipid film is dispersed. The resulting suspension will contain liposomes ranging in size, which may then fractionated to remove undesirable sizes, if necessary. This fractionation may be effected by column gel chromatography, centrifugation, ultracentrifugation or by dialysis, as well known in the art. The above method of preparation of liposomes is representative of a possible procedure only. Those skilled in the art will appreciate that there are many different methods of preparing liposomes, all of which are deemed to be encompassed by the present disclosure.

Applications

Potential applications include any condition for which localized nerve or neural element blockade is desirable, including both local anesthesia and/or local analgesia, motor blockade, and local anesthesia for other medical purposes. Uses include preoperative, intraoperative and postoperative administration to reduce pain during and after an operation or procedure. The benefits are especially significant for plastic surgical procedures and procedures necessitating intense analgesia where prolonged local analgesia will reduce potential morbitities and enhance and improve outcome.

Additional applications include use in trauma patients where tissue damage has occurred as a result of laceration, broken bones or connective tissue strains and tears. Uses may also include treatment of pain due to snake or insect bite, or for pain due to medical conditions such as pancreatitis or kidney stones. These formulations can also be used for the management of various forms of persistent pain, such as postoperative pain, sympathetically maintained pain, complex regional pain syndrome, neuropathic pain and other forms of chronic pain. The aforementioned applications of the methods of the invention are merely mentioned as examples, and additional applications for both human and veterinary practice will be immediately apparent to the artisan.

Local Anesthesia may be used to block pain by targeting specific nerves, as described in Zenz, Panhans, Niesel, Kreuscher, Regional Anesthesia, Year Book Medical Publishers, Inc., Chicago (1988) and Adriani, Labat's Regional Anesthesia, Warren H. Green, Inc., St. Louis, (1985), both of which are incorporated by reference herein in their entireties. Before, during or after surgery, pain may be blocked using local anesthetic agents (existing in a number of forms including EDLA) applied by various techniques known in the art to the following areas of the body: Superficial and/or Deep cervical plexus block in the neck, the Brachial Plexus by interscalene, supraclavicular, infraclavicular, and axillary approaches, the musculocutaneous nerve in the upper extremity, nerves in the elbow region (ulnar nerve, median nerve, radial nerve, lateral antebrachial cutaneous nerve); the nerves in the wrist area (ulnar, median, radial); the Lumbosacral Plexus (Psosas compartment, Lumbar plexus, Sciatic nerves: common peroneal nerve, superficial and deep peroneal nerves, anterior tibial nerve, sural nerve, anterior tibial nerve, musculocutaneous nerve, Tibial nerve); Knee joint nerves (common peroneal, tibial, saphenous); Lumbar Epidural, Cervical, Thoracic and Lumbar Spinal nerve roots, Intercostal nerves, Thoracic Spinal nerves, Spinal Accessory nerve, hypoglossal nerve; lateral femoral cutaneous nerve, suprascapular nerve femoral nerve, Obturator nerve, sacral nerves; Paracervical and Pudendal blocks in Obstetrics.

The following are the nerves susceptible to blockade in the area of pain therapy, specifically Sympathetic blockade: Stellate ganglion, Celiac plexus, Lumbar sympathetic, splanchnic nerves, vagus nerve. The head area includes: The Gasserian ganglion, sphenopalatine ganglion, posterior superior alveolar nerve, infraorbital and anterior superior alveolar nerves, inferior alveolar nerve, lingual nerve, superior laryngeal nerve, inferior or recurrent laryngeal nerve, branches of the ophthalmic nerve (lacrimal, frontal, and nasociliary), mandibular nerve, ethmoidal nerve, mental nerve, lingual nerve, facial nerve, glossopharyngeal nerve, the supraorbital and supratrochlear nerves; The maxillary nerve and palatine nerves; infraorbital, mental, occipital nerves, myofascial trigger points and intercostal block (blockade of the thoracic spinal roots, dorsal branch, ventral branch at angle of rib, ventral branch in posterior axillary line; dorsolateral intercostal block). Inguinal and Iliohypogastric nerves; cervical plexus; phrenic nerve; Peridural block (segmental, continuous epidural block, caudal and subarachnoid block), neuraxial block.

In certain embodiments, the formulation comprises microcapsules comprised of local anesthetic (e.g., bupivacaine) and a biocompatible, biodegradable polymer. In certain preferred embodiments, the polymer is a poly(lactide-co-glycolide). In certain preferred embodiments, the polymer is a 65:35 DL copolymer of lactic and glycolic acid having an inherent viscosity from about 0.25 to about 0.42 dL/g and a molecular weight of about 40 kDa. In other preferred embodiments, the formulation comprises microspheres comprising the local anesthetic, optional augmenting agent, and a polymer such as 65:35 DL copolymer of lactic and glycolic acid having a molecular weight from about 40 kDa to about 120 kDa. In certain other embodiments, the molecular weight of the polymer is about 120 kDa. In other embodiments, the formulation includes a mixture of microspheres utilizing polymers of different molecular weights, e.g., from about 20 kDa to about 120 kDa.

In certain preferred embodiments where the formulation is used for subcutaneous or intramuscular injection, the formulation provides a concentration of bupivacaine free base from about 2.25 mg/ml to about 36.0 mg/ml and provides a unit dose of bupivacaine free base from about 22.5 mg to about 360 mg, said formulation providing an onset of local analgesia and/or local anesthesia at the site of administration which occurs less than about 2 hours after administration, and a duration of effect which lasts for at least about 2 days after administration. In certain preferred embodiments where the formulation is used for subcutaneous or intramuscular injection, the formulations and methods include microspheres, e.g., in the medium at a concentration of about 6.25 mg/ml with about 16 ml of said medium at a strength of about 4.5 mg/ml of bupivacaine. In certain other preferred embodiments, the formulations and methods further comprise microspheres contained in the medium at a concentration of about 12.5 mg/ml with about 8 ml of said medium at a strength of about 9 ng/ml bupivacaine. In certain preferred embodiments where the formulation is used for subcutaneous or intramuscular injection, the formulations and methods further comprise dexamethasone, e.g., at a concentration from about 2.5 mcg/ml to about 10.0 mcg/ml dexamethasone. In certain preferred embodiments, the formulations and methods include microspheres at a concentration of about 25.0 mg/ml with about 4 ml of said medium at a strength of about 18 mg/ml bupivacaine.

Methods of Administration

The formulations of the present invention preferably provide an extended duration of effect in the localized area to be treated. For example, it would be desirable that such a formulation provides localized analgesia, localized numbness (anesthesia), or localized pain relief to the site of administration for a period of one day, two days, three days, or longer. The formulations can therefore, of course, be modified in order to obtain such a desired result.

The formulations of the present invention may be administered by injection, infiltration or infusion, which includes but is not limited to infiltration into muscle, facial, subcutaneous and cutaneous tissue of incisional or damaged (e.g., lacerated) tissue. Intra-articular administration is also contemplated. These applications may be post-surgical (e.g., incisions including laparotomy and laparoscopy) and post-trauma (e.g., laceration). Specific indications could include infiltration in tissue approximating surgical incisions for hernia repair, iliac crest harvest site, breast surgery, C-section, episiotomy and general abdominal incisions (cholecystectomy, colon resection/repair, gastric repair, etc.).

The microspheres and other injectable substrates described herein may be incorporated into a pharmaceutically acceptable vehicle (e.g., water) to prepare a suspension for injection. The final reconstituted product viscosity may be in a range suitable for the route of administration. In certain instances, the final reconstituted product viscosity may be such that would be considered suitable for subcutaneous or intramuscular injection at the desired site, e.g., about 5-15 cps, preferably about 8-12 cps. A preferred diluent for microspheres contains approximately 5% mannitol or 0.9% sodium chloride to maintain isotonicity; from about 0.01% to about 0.5% Polysorbate 80 (or Polysorbate 20) as a dispersant; and from about 0.5% to about 3.0% sodium carboxymethylcellulose (or methylcellulose) for the desired viscosity.

The microspheres of the invention are preferably incorporated into a unit dose in a size range suitable for injection into a desired site of administration by injection, infiltration, infusion and the like. For administration by injection and/or infiltration or infusion, the formulations according to the invention may be suspended (e.g., for microspheres), or dissolved (e.g., for immediate release local anesthetic components of the formulations), in any art-known vehicle suitable for microsphere dispersion and suspension, and subsequent injection, and/or infiltration or infusion. Such vehicles include, simply by way of example, isotonic, buffered or unbuffered vehicles containing suitable surfactant and thickening agents and the like, and may optionally include any other art known ingredients or agents, e.g., colorants, preservatives, antibiotics, epinephrine, and other art known ingredients. A more complete listing of art-known vehicles for administration of formulations by systemic administration and/or local injection and/or infiltration is provided by reference texts that are standard in the art, for example, REMINGTONS PHARMACEUTICAL SCIENCES, 16th Edition, 1980 and 17th Edition, 1985, both published by Mack Publishing Company, Easton, Pa.

In a preferred method of administration, the microspheres are administered by injection into a site where local anesthetic agent is to be released. Such administration may be accomplished using a syringe and needle or a trochar. The formulation described herein can also be used to administer local anesthetic agents that produce modality-specific blockade, as reported by Schneider, et al., Anesthesiology, 74:270-281 (1991), or that possess physical-chemical attributes that make them more useful for sustained release than for single injection blockade, as reported by Masters, et al., Soc. Neurosci. Abstr., 18:200 (1992), the teachings of which are incorporated herein.

A suspension of microspheres prepared in a form suitable for subcutaneous injection can be injected using methods well known in the art. The use of a needle is acceptable. The chosen needle is one that is small in bore (large) gauge as possible, and as long as is necessary. Commonly, for subcutaneous administration, a 20-23 gauge, 1″ needle is used. For the microparticles used in the present invention, one should allow for increased bore size (e.g., up to 18 gauge). This also allows for the puncturing needle to be removable, being encased in a plastic infusion catheter. For some procedures, “skinny” needles may be used. Such needles have the same bores but are longer, and hence look “skinny.” For locations such as pericardial, the gauges for the skinny needle are the same but the needles may be up to 3-4 inches long.

Microparticles (e.g., microcapsules) according to the invention that are suitable for deposit at a site in a patient in need of local anesthesia or analgesia can optionally be prepared in lyophilized form, e.g., for rehydration prior to use. The formulation, e.g., in the form of lyophilized particles is also desirably prepared in unit dosage form that is sterilized and provided in a container including an amount of such lyophilized particles sufficient to induce prolonged local anesthesia in at least one patient upon suspension in a solution acceptable for deposit into a patient.

Local Anesthetics

Local anesthetic agents which may be included in the formulations and methods of the present invention include, simply by way of example, bupivacaine, ropivacaine, dibucaine, procaine, chloroprocaine, prilocaine, mepivacaine, etidocaine, tetracaine (including but not limited to N-butyl tetracaine), lidocaine (including but not limited to N-beta-phenylethyl lidocaine), ethyl aminobenzoic acid, oxyburocaine, oxesazeine, benzoxazinate, proparacaine, benzocaine, butamben, halothane, isoflurane, enflurane, methoxyflurane xylocaine and the normal crystalline forms of bupivacaine, as well as anesthetically active derivatives, analogs and mixtures thereof. The local anesthetic can be in the form of a salt, for example, the hydrochloride, bromide, acetate, citrate, carbonate or sulfate. More preferably, the local anesthetic agent is in the form of a free base. A preferred local anesthetic agent is bupivacaine free base.

In certain embodiments, the bupivacaine free base comprises one or more crystalline bupivacaine polymorphs. In certain preferred embodiments, the microspheres are microcapsules which contain crystalline polymorphs of bupivacaine. Comparison of the X-ray diffraction pattern of the bupivacaine base raw material with the altered crystal form of bupivacaine in microspheres shows that there is a difference in the diffraction patterns. Major differences are observed at °2 Theta of approximately 7.5, 12.5 and 20). The melting transition of bupivacaine base (as shown in the DSC thermograms) has been identified herein as 107.6° C. for bupivacaine base (raw material). The melting transitions for the crystalline bupivacaine polymorphs have been identified as 94.4° C. and 100.8° C., corresponding to at least two polymorphs. Powder X-ray diffraction of such crystalline polymorphs of bupivacaine provides a peak of about 400 to about 600 counts/s (preferably about 500 counts/s) at °2 Theta of from about 7 to about 9; substantially no peak at °2 Theta of about 12.5 (e.g., about 100 counts/s); and a peak from about 1300 to about 1500 counts/s at °2 Theta of about 20 to about 21. This is differentiated from substantially no peak (e.g., about 0 counts/s) at °2 Theta of from about 7 to about 9; a peak of about 700 counts/s at °2 Theta of about 12.5 (e.g., about 100 counts/s); and a peak of about 750 counts/s at °2 Theta between about 19 and 20, with substantially no peak (e.g., 200 counts/s) at °2 Theta between 20 and 21, for the bupivacaine raw material.

The novel crystalline polymorph(s) of bupivacaine of the invention may also be characterized as exhibiting essentially the following x-ray diffraction properties set forth in

TABLE 1
X-ray Diffraction Properties of Bupivacaine Polymorph

| d-spacing (A) | Relative Intensity (%) | Angle (°2□) |
|---|---|---|
| 11.08-11.11 | 37.82-39.85 | 7.95-7.97 |
| 8.90-8.92 | 99.89-100 | 9.91-9.93 |
| 7.47-7.53 | 11.36-14.00 | 11.75-11.84 |
| 5.04-5.06 | 16.21-21.85 | 17.53-17.58 |
| 4.71 | 12.14-19.14 | 18.81-18.83 |
| 4.50-4.51 | 32.56-40.35 | 19.68-19.71 |
| 4.36 | 87.52-100 | 20.34-20.36 |
| 4.30 | 84.46-97.73 | 20.62-20.63 |
| 4.22-4.23 | 22.86-29.53 | 21.00-21.05 |
| 4.14-4.15 | 18.65-26.45 | 21.40-21.45 |
| 4.06-4.07 | 13.94-21.20 | 21.83-21.86 |
| 3.85-3.86 | 34.52-46.17 | 23.04-23.08 |
| 3.73 | 14.15-22.05 | 23.84-23.85 |

In certain preferred embodiments, the onset of analgesic activity of the formulations is shortened via the concurrent or combined administration of an effective amount of a relatively fast-acting local anesthetic, e.g., lidocaine, in immediate release form. In such instances, the onset of analgesic activity may be anywhere from instantaneous to less than about 2 hours after administration, preferably from about 0 to about 5 minutes after administration of the formulation. The concentration of lidocaine ranges, e.g., from about 0.5% to about 2%. For example, a further embodiment of the present invention includes mixing ready-to-use and/or concentrated solutions of lidocaine (e.g., 20%) in a diluent, whereby the performance of suspended microspheres depends upon diluent qualities (e.g.; dilution effect of lidocaine while maintaining desirable suspending vehicle properties) after the lidocaine and diluent have been mixed. In another embodiment of the present invention, lidocaine 10% (concentrated) is combined with the diluent to minimize dilution of the diluent, thereby achieving therapeutic levels of the lidocaine. In yet another embodiment of the present invention, lidocaine 20% (concentrated) is combined with the diluent to further minimize dilution of the diluent, thereby achieving therapeutic levels of the lidocaine. Preferably, the optimal range of viscosity of such mixed solutions ranges from about 8 cSt to about 12 cSt.

Augmentin Agent

In certain preferred embodiments, the local anesthetic formulations also include an amount of an augmenting agent, e.g., a glucocorticosteroid or nonglucocorticoid agent, that may be provided in any form suitable for administration. Augmenting agents according to the invention are compositions or compounds that prolong the duration of local anesthesia and/or enhance the effectiveness of local anesthetic agents when delivered to the site of local anesthetic administration before, simultaneously with or after the local anesthetic is administered. The augmentation of efficacy provided by the use of the augmenting agent cannot be predicted based on in vitro release (dissolution) of the bupivacaine in controlled release form. The inclusion of the augmenting agent within the controlled release formulations of the invention does not substantially alter or prolong the in-vitro dissolution rate of bupivacaine agent from the formulation; yet, the same formulation when administered in-vivo provides a rapid onset of local anesthesia and a significant increase in the time period of local anesthesia at the site of administration. The optimal concentration of augmenting agent for human clinical use may also be readily determined by routine animal screening-as described hereinbelow, and further adjusted, where indicated, by routine clinical experience.

The augmenting agents disclosed herein may be administered prior to, along with, or after administration, e.g., topical application, infiltration and/or injection of the local anesthetic agent in sustained release form, in each case with a substantial prolongation of local anesthesia in-vivo. In one embodiment, local anesthetic and augmenting agents are administered simultaneously in microspheres containing both the local anesthetic and the augmenting agent in a single medium for injection of infiltration. Alternatively, the local anesthetic and augmenting agent may be administered in the form of, e.g., separate microspheres suspended in a single (or separate) medium(s) suitable for injection or infiltration. In a further embodiment, simply by way of example, administration of controlled release microspheres with combined local anesthetic and vasoconstrictor agent can also be followed by one or more additional administrations of such combination formulation and/or of microspheres including as the active agent only local anesthetic or only vasoconstrictor agent.

The microspheres according to the invention can be administered alone or in combination with a solution including a glucocorticoid or non-glucocorticosteroid augmenting agent in an amount effective to prolong the duration of local anesthesia. Alternatively, in preferred embodiments the microspheres include an amount of an augmenting agent effective to prolong the duration of local anesthesia. In another alternative, one or more augmenting agents can be administered before, simultaneously with or after administration of the sustained release local anesthetic, wherein the augmenting agent is formulated into a separate microsphere formulation for sustained release. The controlled release rate for the augmenting agents may be the same as or different than the controlled release rate for the local anesthetic. The separate microsphere can be administered in a single injection, i.e., in a single injection vehicle, or in separate injections simultaneously or at different times. In a further embodiment, it has been found that additional dose of augmenting agent may also be administered as an injectable solution, in an injectable carrier or in a sustained release carrier to the nerve to be blockaded after the sustained release local anesthesia has worn off, to reactivate the initial local anesthesia without the co-administration of additional local anesthetic.

In those embodiments of the invention directed to formulations where the augmenting agent is included in the formulation, the augmenting agent may be included in controlled release form or in immediate release form. The augmenting agent may be incorporated into any pharmaceutically acceptable carrier. For example, the augmenting agent may be incorporated into or onto the surface of the microcapsules, which include the local anesthetic, or may be incorporated into separate particles suitable for administration (e.g., microspheres, microcapsules, etc.). Alternatively, the augmenting agent may be incorporated, either in controlled release form or in immediate release form, into a pharmaceutically acceptable aqueous medium suitable for infiltration or injection (separately or together with the microcapsules containing the local anesthetic).

In certain embodiments of the invention, the augmenting agent can be from one or more of the following general types or classes of agents, including glucocorticosteroid agents, alkalinizing agents, non-glucocorticoid steroids such as, e.g., neuroactive steroids and/or steroid or nonsteroid modulators of gamma amino butyric acid (“GABA”) receptors, modulators of ionic transport across cell membranes, including, e.g., modulators of membrane transport of monovalent and divalent metal ions such as, for example, blockers or enhancers of sodium, potassium and/or calcium transport across cell membranes, antipyretic agents, adrenergic receptor agonists or antagonists, such as alpha-2 receptor agonists, tubulin binding agents, including, e.g., agents that are capable of either causing formation or disruption of intracellular microtubules, osmotic polysaccharides, agonists and antagonists of potassium ATP channels, i.e., able to open or close potassium ATP channels, Na, K-ATPase inhibitors and enhancers, neurokinin antagonists, PLC (i.e., phosphatidylinositol-specific phospholipase C) inhibitors, inhibitors of leukocyte glucose metabolism and anti-convulsants. The augmenting agent can also be an analeptic, a tranquilizing agent, an ataretic, an antidepressant, an anti-seizure agent, leukotriene and prostaglandin agonists and inhibitors, phosphodiesterase agonists and inhibitors, e.g., based on cAMP, and combinations of any of the foregoing. Vasoconstrictive agents provided in controlled release form also provide for unexpected and surprising augmentation of duration and potency of local anesthetics relative to immediate release forms of vasonstrictive agents heretofore known to the art. The aforementioned types of augmenting agents may to used alone or in any mixture or combination of each such agent to provide effective augmentation of local anesthesia where desired.

In one embodiment, the augmenting agent is any art-known glucocorticosteroid agent, such as, simply by way of example, dexamethasone, cortisone, prednisone, hydrocortisone, beclomethasone dipropionate, betamethasone, flunisolide, methylprednisone, paramethasone, prednisolone, triamcinolone, alclometasone, amcinonide, clobetasol, fludrocortisone, diflorasone diacetate, fluocinolone acetonide, fluocinonide, fluorometholone, flurandrenolide, halcinonide, medrysone and mometasone, ropivicaine and pharmaceutically acceptable mixtures and salts thereof and any other derivatives and analogs thereof.

When a glucocorticosteroid agent is included in the controlled release formulation microcapsules comprising local anesthetic (e.g., microcapsules), it has been found that useful loadings of glucocorticosteroid agent are, e.g., from 0.005% to 30% by weight of the substrate.

When the glucocorticosteroid agent is included with a suitable vehicle in which microparticles comprising local anesthetic are suspended, the glucocorticosteroid agent is present, for example, in a weight percent relative to the local anesthetic varying from about 0.005% to about 15%.

In another embodiment, the augmenting agents include an alkalinizing agent. The alkalinizing augmenting agents used herein preferably raise the pH of the medium in which the local anesthetic agents in sustained release form are present (e.g., either an injection medium or the environment at the site of injection) to provide a pH from about 6.0 to about 8.5, preferably from about 7.5 to about 8.5. Preferably, the alkalinizing agent may be, for example, a carbonate buffer such as sodium carbonate. Of course, any other alkalinizing agent that is pharmaceutically acceptable for localized injection or infiltration may also be effectively employed. The augmenting agents also include non-glucocorticoid steroids such as e.g., androgens, such as testosterone and its active derivatives, analogs and metabolites; estrogens, such as estradiol and its active derivatives, analogs and metabolites and progestins, such as progesterone and its active derivatives, analogs and metabolites and mixtures of any of these.

In yet another embodiment, the augmenting agents are neuroactive steroids, such as, e.g., one or more of the class of anesthetic steroids. Neuroactive steroids useful as augmenting agents according to the invention also include those that modulate GABA receptors. Preferred neuroactive steroids include, simply by way of example, althesin and its main component, alphaxalone and active analogs, derivatives and mixtures thereof, as well as 5-alpha-pregnane-3 alpha-21-diol-20-one (tetrahydro-deoxycorticosterone or THDOC) and/or allotetrahydrocortisone (the 17-beta configuration); and dehydroepiandrosterone (“DHE”) and active analogs, derivatives and mixtures thereof. Preferably, the neuroactive steroids are present as an additive in the vehicle carrying the microspheres in a concentration ranging from about 0.01% to about 1% by weight, and most preferably from about 0.05% to about 0.5% by weight.

The augmenting agents also include non-steroidal modulators of GABA receptors, including those that are capable of potentiating the inhibitory effects of GABA on those receptors. Preferably, these include the benzodiapenes, e.g., diazepam as well as its active derivatives, analogs and metabolites and mixtures thereof. More preferably, the diazepam is present as an additive in the vehicle in a concentration ranging from about 0.01% to about 1% by weight, and most preferably from about 0.05% to about 0.5% by weight. Of course, the artisan will appreciate that the potency of benzodiazapenes varies widely, and will adjust these concentration ranges accordingly for other benzodiazapenes, relative to the potency of diazepam.

In yet another aspect of the invention, the augmenting agent is a modulator of ionic transport across cell membranes. Monovalent and multivalent metal ion transport can be modulated. Agents include, e.g., sodium, potassium and calcium channel modulators (e.g., nifedipine, nitrendipine, verapamil, etc.). In preferred embodiments, these also include, but are not limited to, aminopyridine, benzamil, diazoxide, 5,5 diphenylhydantoin, minoxidil, tetrethylammonium and valproic acid. Such augmenting agents, which can be used in accordance with the present invention include naturally occurring site 1 sodium channel blockers, such as tetrodotoxin, saxitoxin, decarbomoyl saxitoxin, neosaxitoxin, and other similarly-acting, structurally homologous toxins. Further, combinations of these toxins with further agents such as vasoconstrictors, glucocorticoids, alpha agonists (epinephrine, phenylephrine), beta-blockers (propranolol) and mixed central-peripheral alpha-2 agonists (clonidine), and/or adrenergic drugs, may be used as the augmenting agent. Such combinations are described in International Patent Publication No. WO 98/51290, which is hereby incorporated by reference. The inclusion of amphiphilic and/or lipophilic solvents in the formulations of the invention, together with the use of such toxins, is contemplated as a further augmenting alternative with respect to the present invention, as described in International Patent Publication No. WO 98/51290. Preferably, the ion transport modulating agent is present as an additive in the vehicle carrying the microspheres in a concentration ranging from about 0.01 to about 5 percent by weight, and most preferably from about 0.05 to about 1.5 percent by weight.

Augmenting agents also include, e.g., antipyretic agents such as aminopyrine, phenazone, dipyrone, apazone, phenylbutazone and derivatives and analogs thereof Aminopyrine is preferably included in the vehicle containing the microspheres in a concentration ranging from about 0.01 to about 0.5 percent and in a more preferred embodiment the concentration ranges from about 0.05 to about 0.5 percent, by weight.

Other preferred augmenting agents include, e.g., adrenergic receptor modulators, such as alpha-2 receptor agonists, can also be used as augmenting agents. Simply by way of example, the alpha-2 receptor agonist clonidine provides useful augmentation of local anesthesia, although any other art known alpha-2 receptor modulators capable of augmenting local anesthesia according to the invention may be used. Clonidine is preferably included in the vehicle containing the microspheres in a concentration ranging from about 0.01% to about 0.5% preferred embodiment the concentration ranges from about 0.05% to about 1%, by weight.

Tubulin binding agents that are capable of promoting the formation or disruption of cytoplasmic microtubules may be employed as augmenting agents according to the invention. Such agents include, for example, colchicine and the vinca alkaloids (vincristine and vinblastine), taxol as well as active derivatives, analogs metabolites and mixtures thereof. Of course, some agents may be classified in more than one category, thus, for example, colchicine is also known to inhibit glucose metabolism in leukocytes. Colchicine is preferably included in the vehicle containing the microspheres in a concentration ranging from about 0.01 to about 1.0 percent and in a more preferred embodiment the concentration ranges from about 0.05 to about 0.5 percent, by weight.

Additional augmenting agents, which may be used in conjunction with the present invention, include vanilloids such as naturally occurring and synthetic capsaicin, resiniferotoxin, and the like.

Osmotic polysaccharides are also able to be used as augmenting agents. In one preferred embodiment, the osmotic polysaccharide includes dextran. More preferably, the dextran augmenting agents according to the invention have a molecular weight ranging from about 20 kDa through about 200 kDa, or greater. A solution containing dextran in a form suitable for injection or infiltration into a desired site in a patient is preferably buffered to a pH ranging from about 3.0 to about 8.5, but in a preferred aspect is buffered to a pH ranging from about 7.0 to about 8.5.

Other preferred embodiments of the invention provide for potassium-ATP channel agonists for use as augmenting agents. A preferred potassium-ATP channel agonist is, e.g., diazoxide, as well as its active derivatives, analogs, metabolites and mixtures thereof that are useful as augmenting agents.

Sodium/potassium ATPase inhibitors are also preferred as augmenting agents according to the invention. Preferably, the sodium/potassium ATPase inhibitors are cardiac glycosides that are effective to augment local anesthesia. Cardiac glycosides that are useful according to the invention include, e.g., oubaine, digoxin, digitoxin and active derivatives, analogs and metabolites and mixtures of any of these.

Additionally, augmenting agents which may be used in accordance with the present invention include, e.g., neurokinin antagonists, such as, e.g., spantide and other peptide inhibitors of substance P receptors that are well known to the art, e.g., as are listed in Receptor and Ion Channel Nomenclature Supplement, Trends in Pharmacological Sciences 18:64-65, the disclosure of which is incorporated by reference herein in its entirety. PLC (i.e., phosphatidylinositol-specific phospholipase C) inhibitors such as, e.g., 1-[6-[[17-beta-3-methoxyestra-1,3,5(10)-triene-17-yl]amino]hexl]-1-H-pyrrole-2,5-dione, and anti-seizure agents and agents that stabilize cell membrane potential, such as, e.g., benzodiazepines, barbiturates, deoxybarbiturates, carbamazepine, succinamides, valproic acid, oxazalidienbiones, phenacemide and active derivatives, analogs and metabolites and mixtures thereof. Preferably, the anti-seizure augmenting agent is phenytoin, and most preferably is 5,5-diphenylhydantoin.

Locally acting vasoconstrictive agents also provide effective augmentation of local anesthesia that may be superior to that provided by immediate release vasoconstrictive agents. While not wishing to be bound by any hypothesis as to how vasconstrictive agents in controlled release form might greatly prolong local anesthetic activity, it is believed that controlled release vasoconstrictor agents provide a controlled and non-toxic vasoconstrictor activity that reduces the rate of local anesthetic washout from the treated tissue area to prolong the presence of effective concentrations of local anesthetic in the tissue. It is known to the art that vasoconstrictors, e.g., epinephrine, prolong local anesthetic activity for, at best, about 1 hour and that if excessive amounts of epinephrine or other vasoconstrictor is administered in an attempt to further prolong local anesthesia, local circulation may be so disrupted as to cause tissue necrosis and gangrene. Controlled release vasoconstrictor agents can achieve local tissue concentrations that are safe and effective to provide vasoconstrictor activity effective to substantially prolong local anesthesia. More unexpectedly, the local circulatory bed, i.e., blood vessels, remains responsive to the vasoconstrictor agent for prolonged periods, e.g., receptor desensitization or smooth muscle fatigue or tolerance does not prevent the prolongation effect. The gradual release from a controlled release formulation also serves to greatly reduce the risk of toxic reactions such as, e.g., localized tissue necroses.

The previously discussed vasoconstrictive augmenting agents can be administered before, simultaneously with or after the administration of local anesthetic. In one embodiment of the invention, at least a portion of the vasoconstrictive agent is formulated in the controlled release formulation together with local anesthetic. In another embodiment, the vasconstrictive agent is prepared in one or separate controlled release formulations.

Vasoconstrictor agents which may be used as augmenting agents in accordance with the invention include, but are not limited to, catecholamines e.g., epinephrine, norepinephrine and dopamine as well as, e.g., metaraminol, phenylephrine, methoxamine, mephentermine, methysergide, ergotamine, ergotoxine, dihydroergotamine, sumatriptan and analogs, and alpha-1 and alpha-2 adrenergic agonists, such as, e.g., clonidine, guanfacine, guanabenz and dopa (i.e., dihyrdoxyphenylalanine), methyldopa, ephedrine, amphetamine, methamphetamine, methylphenidate, ethylnorepinephrine ritalin, pemoline and other sympathomimetic agents, including active metabolites, derivatives and mixtures of any of the foregoing.

In a more preferred embodiment, at least a portion of any of the augmenting agents enumerated above are included in the sustained release formulation, in combination with a local anesthetic agent or agents in a concentration ranging from about 0.01 to about 30 percent or more, by weight, relative to the weight of the formulation. Preferably, the vasoconstrictor is included in a sustained release formulation in an amount ranging from about 0.005 percent to about 20%, and more preferably, from about 0.05 percent to about 5 percent, by weight, relative to the total weight of the formulation. When a vasoconstrictor is present in the injection vehicle in immediate release form, it is present in amounts ranging from about 0.01% to about 5 percent, or more, by weight, relative to the injection vehicle. The vasoconstrictor can also be provided in a ratio of local anesthetic, e.g., bupivacaine to vasoconstrictor, ranging from about 10:1 to about 20,000 and preferably from about 100:1 to about 2000:1 and from about 500:1 to about 1500:1.

The artisan will also appreciate that other augmenting agents according to the invention broadly include any other types and classifications of drugs or active agents known to the art. Such augmenting agents are readily identified by routine screening as discussed hereinbelow using animal sensory and motor quantitation protocols well known to the art.

The artisan will also appreciate that the amounts of augmenting agent and local anesthetic will vary depending upon the relative potency of the agents selected, the depth and duration of local analgesia, local anesthesia and/or local nerve blockade is desired. The optimal concentration and/or quantities or amounts of any particular augmenting agent, whether present in the injection vehicle, separately administered before, during or after local anesthesia is induced or whether included in the microsphere formulation, may be adjusted to accommodate variations in the treatment parameters. Such treatment parameters include the polymer composition of a particular microsphere preparation, the particular local anesthetic utilized, and the clinical use to which the preparation is put, in terms of the site treated for local anesthesia, the type of patient, e.g., human or non-human, adult or child, and the type of sensory stimulus to be anesthetized.

Further, the concentration and/or amount of any particular augmenting agent for a given formulation may be readily identified by routine screening in animals, e.g., rats, by screening a range of concentration and/or amounts of augmenting agent using the hotplate foot withdrawal assay and/or motor function assay described hereinbelow.

When the augmenting agent is included in the sustained release substrates (e.g., microparticles) comprising local anesthetic, it has been found that useful loadings of augmenting agent are from about 0.001% to about 30% by weight of the substrate or preferably from about 0.01% to about 5% by weight of the substrate. When the augmenting agent is included in controlled release substrates (e.g., microspheres) without local anesthetic, it has been found that useful loadings of augmenting agent are from about 0.001% to about 90%, or more, by weight of the substrate, or preferably from about 0.001% to about 30% by weight of the substrate or more preferably from about 0.01% to about 5% by weight of the substrate.

When the augmenting agent is included as part of the (aqueous) injection medium, the augmenting agent may be present in a weight percent relative to the local anesthetic varying from about 0.01% to about 15%.

The examples demonstrate that the above-described augmenting agents prolong the duration of local anesthesia in-vivo and do not significantly alter the time course of release of the local anesthetic in-vitro.

Additional Active Agents

The formulations of the present invention may further incorporate one or more additional active agents, which may provide similar therapeutic effects, additive therapeutic effects, or different therapeutic effects. The additional active agent(s) may be a pharmaceutically active agent, such as a drug and/or diagnostic substance for human or veterinary use. For example, in addition to the local analgesia provided by the local anesthetic, a drug of a different class than those traditionally associated with local anesthetic properties but which can provide analgesia may be included in the formulation. Such drugs include but are not limited to opioids such as morphine, fentanyl, cocaine, codeine and agents, which, for example, can provide regional blockade of nociceptive pathways (afferent and/or efferent).

Additional pharmaceutically active agents that can be incorporated into the formulations of the invention, include, e.g., antibiotics such as sulfisoxazole, penicillin G, ampicillin, cephalosporins, amikacin, gentamicin, tetracyclines, chloramphenicol, erythromycin, clindamycin, isoniazid, rifampin, and derivatives, salts and mixtures thereof; antifungals such as amphotericin B, nystatin, ketoconazole; antivirals such as acyclovir, amantadine; anticancer agents such as cyclophosphamide, methotrexate, etretinate and other art known anti-infective or antitumor agents or combinations thereof.

An active agent can also be an enzyme, antibody, antigen or other biological protein or peptide for pharmaceutical and/or diagnostic use or combinations thereof. An active agent may also be, simply by way of example, any art known agent, e.g., a polypeptide or peptide derivative effective to protect or regenerate cartilage and/or connective tissue.

Diagnostic agents that can be administered as an additional agent intra articularly according to the invention include, e.g., dyes, vital dyes, radio-opaque dyes, magnetic resonance imaging dyes, electron spin dyes, radio-isotope labeled moieties and others readily apparent to the artisan, or combinations thereof. In a preferred embodiment, the formulation can be prepared, e.g., to include any art-known nontoxic and radio-opaque dye, e.g., an iodine compound and the like, to aid in the visualization of the site for improved accuracy of administration and where desirable, to monitor the location of any controlled release material remaining at the site at a later time. In another embodiment, at least a portion of such optional radio-opaque dye is present in the suspending vehicle to assist in the localization of the site of injection.

Prodrugs are well known in the art and include inactive drug precursors which, when exposed to high temperature, metabolizing enzymes, cavitation and/or pressure, in the presence of oxygen or otherwise, or when released from the formulations in accordance with the invention (e.g., microcapsules), will form active drugs in the intercellular or intracellular environment. Suitable prodrugs, which may be included as additional active agents will be apparent to those skilled in the art.

Examples of antibodies that can be incorporated into the formulations of the invention generally include industrial antibodies as well as antibodies and derivatives of antibodies for use in biotechnological process as well as antibodies for diagnostic and therapeutic purposes. Such antibodies include, for example, IgA, IgD, IgG, IgE, IgM, and combinations thereof, in the form of monoclonal, polyclonal and recombinant antibodies, catalytic antibodies and antigen-binding antibodies. Further, fragments of antibodies can be incorporated, together with or separately from, intact antibodies. For example, antibody fragments include light and/or heavy chains, and combinations of light chains or heavy chains, as well as the Fab, Fv, Fc, Fd and smaller fragments, such as active portions of the variable region and non-naturally occurring combinations of such fragments and/or light and heavy chains or combinations thereof. Recombinant polypeptides with antibody activity can also be incorporated into microparticles by this method, as can engineered antibodies or antibodies or antibody fragments that are linked to other molecules, e.g., drugs, prodrugs and/or diagnostic or analytic label moieties or combinations thereof.

Examples of genetic materials that can be incorporated, include, e.g., nucleic acids such as RNA and DNA, of either natural or synthetic origin, including recombinant RNA and DNA and antisense RNA and DNA as well as chemical derivatives of these nucleic acids, e.g., phosphonamides. Types of genetic material that may be incorporated include, for example, genes carried on expression vectors such as plasmids, phagemids, cosmids, yeast artificial chromosomes (YACs), and defective or “helper” viruses, anti-gene nucleic acids, both single and double stranded RNA as well as viral vectors for transforming cells, in vivo or in vitro or for genetic therapy, e.g., retroviral vectors, adenoviral vectors and the like or combinations thereof.

Examples of enzymes that can be incorporated into the formulations of the invention include, generally, enzymes for diagnosis and therapeutic purposes, e.g., ribonuclease, neuramidinase, trypsin, glycogen phosphorylase, amino peptidase, trypsin chymotrypsin, amylase, muramidase, diesterase, glutamic acid dehydrogenase, as well as fibrinolytic enzymes, lysozymes, dextranase and ribozymes or combinations thereof, to name but a few that will be readily apparent to the artisan.

The additional active agent(s) can be either soluble or insoluble in a polymer solvent and may be in any pharmaceutically acceptable state, including liquids, solutions, pastes, solids, and the like, or may be included in, e.g., the microspheres along with the local anesthetic and optional augmenting agent.

Art known methods are also available to assay local tissue concentrations, diffusion rates from microspheres and local blood flow before and after administration of local anesthetic formulations according to the invention. One such method is microdialysis, as reviewed by T. E. Robinson et al., 1991, MICRODIALYSIS IN THE NEUROSCIENCES, Techniques, volume 7, Chapter 1, pages 1-64, incorporated herein by reference in its entirety.

The methods reviewed by Robinson can be applied, in brief, as follows. A microdialysis loop is placed in situ in a test animal. Dialysis fluid is pumped through the loop. When microspheres according to the invention are injected adjacent to the loop, released drugs, e.g., bupivacaine and vasoconstrictor augmenting agents, are collected in the dialysate in proportion to their local tissue concentrations. The progress of diffusion of the active agents can be determined thereby with suitable calibration procedures using known concentrations of active agents. For the vasoconstrictor augmenting agents, decrements and durations of vasoconstriction effects can be measured by clearance rates of marker substances, e.g., methylene blue or radiolabeled albumen from the local tissue.

Definitions or further descriptions of any of the foregoing terminology are well known in the art and may be found by referring to any standard biochemistry reference text such as “Biochemistry” by Albert L. Lehninger. Worth Publishers, Inc. and “Biochemistry” by Lubert Stryer, W. H. Freeman and Company, both of which are hereby incorporated by reference.

It is possible to tailor a system to deliver a specified loading and subsequent maintenance dose by manipulating the percent drug incorporated in the polymer and the thickness and porosity of the encapsulating shell matrix, in addition to varying the form and mixture of local anesthetic (e.g., free base versus salt), and the method of production.

All documents cited herein are incorporated by reference in their entireties for all purposes.

### EMBODIMENTS OF THE INVENTION

In certain embodiments, the invention is directed to a method for providing local analgesia, local anesthesia or nerve blockade in a human, comprising administering at a site in a human a formulation comprising a plurality of microspheres comprising a biocompatible, biodegradable carrier and a local anesthetic effective to provide local analgesia, local anesthesia or nerve blockade at the site of administration in a human which occurs less than 2 hours after first administration, and a duration of local analgesia, local anesthesia or nerve blockade which lasts for at least about 1 day after first administration, wherein the level of local anesthetic at the site of administration is at least 100 times, 150 times, 175 times or 200 times the level of local anesthetic in the systemic blood plasma. The present invention is also directed to formulations utilized in this method.

In certain embodiments, the present invention is directed to the above formulations and methods, wherein said formulation further comprises an augmenting agent in an amount effective to prolong the effect of the local anesthetic for a time period greater than that obtained via administration of said formulation without said augmenting agent such that a duration of local analgesia lasts for at least about 2 days after first administration, wherein the level of augmenting agent at the site of administration is at least 200 times, 250 times or 300 times the level of augmenting agent in the systemic blood plasma.

In certain embodiments, the invention is directed to a method for providing local analgesia, local anesthesia or nerve blockade in a human comprising administering at a site in a human a unit dose of microspheres comprising a biocompatible, biodegradable carrier and bupivacaine or a pharmaceutically acceptable salt thereof, effective to provide local analgesia, local anesthesia or nerve blockade at the site of administration in a human which occurs less than about 2 hours after first administration, and a duration of local analgesia, local anesthesia or nerve blockade which lasts for at least about 1 day after first administration, wherein the mean Cmax of bupivacaine measured by microdialysis in the tissue at the site is from about 35,000 ng/ml to below the toxic concentration at the site of administration. The present invention is also directed to formulations utilized in this method.

In certain embodiments, the present invention is directed to the above formulations and methods, wherein said formulation further comprises an effective amount of dexamethasone or a pharmaceutically acceptable salt thereof to prolong the effect of the bupivacaine for a time period greater than that obtained via administration of said formulation without said augmenting agent such that a duration of local analgesia, anesthesia or nerve blockade lasts for at least about 2 days after first administration, wherein the mean Cmax of dexamethasone measured by microdialysis in the tissue at the site is from about 45 ng/ml to below the toxic concentration at the site of administration.

In certain embodiments, the invention is directed to a method for providing local analgesia, local anesthesia or nerve blockade in a human, comprising administering a unit dose of microspheres comprising a biocompatible, biodegradable carrier and bupivacaine or a pharmaceutically acceptable salt thereof, effective to provide local analgesia, local anesthesia or nerve blockade at a site of administration in a human which occurs less than about 2 hours after first administration, and a duration of local analgesia, local anesthesia or nerve blockade which lasts for at least about 1 day after first administration, wherein the mean Tmax of bupivacaine occurs at a point from about 10 hours to about 45 hours after administration. The present invention is also directed to formulations utilized in this method.

In certain embodiments, the present invention is directed to the above formulations and methods, wherein said formulation further comprise an effective amount of dexamethasone or a pharmaceutically acceptable salt thereof to prolong the effect of the bupivacaine for a time period greater than that obtained via administration of said microspheres without said dexamethasone, such that a duration of local analgesia, anesthesia or nerve blockade lasts for at least about 2 days after first administration, wherein the mean Tmax of dexamethasone occurs at a point from about 5 hours to about 40 hours after administration.

In certain embodiments, the invention is directed to a method for providing local analgesia, local anesthesia or nerve blockade in a human, comprising administering a unit dose of microspheres comprising a biocompatible, biodegradable carrier and bupivacaine or a pharmaceutically acceptable salt thereof, effective to provide local analgesia, local anesthesia or nerve blockade at a site of administration in a human which occurs less than about 2 hours after first administration, and a duration of local analgesia, local anesthesia or nerve blockade which lasts for at least about 1 day after first administration, wherein the mean AUCt of bupivacaine at 96 hours measured by microdialysis in the tissue at the site is from about 2,000,000 ng/ml*h to about 4,000,000 ng/ml*h as measured by microdialysis. The present invention is also directed to formulations utilized in this method.

In certain embodiments, the present invention is directed to the above formulations and methods, wherein said formulation further comprise an effective amount of dexamethasone or a pharmaceutically acceptable salt thereof to prolong the effect of the bupivacaine for a time period greater than that obtained via administration of said microspheres without said dexamethasone, such that a duration of local analgesia, anesthesia or nerve blockade lasts for at least about 2 days after first administration, wherein the mean AUCt of dexamethasone at 96 hours measured by microdialysis in the tissue at the site is from about 800 ng/ml*h to about 3,000 ng/ml*h.

In certain embodiments, the present invention is directed to the above formulations and methods, wherein the mean Cmax of bupivicaine in the plasma is below about 250 ng/ml.

In certain embodiments, the present invention is directed to the above formulations and methods, wherein the mean Cmax of dexamethasone in the plasma is below about 0.50 ng/ml.

In certain embodiments, the present invention is directed to the above formulations and methods, wherein the mean Tmax of bupivicaine in the plasma is from about 25 to about 50 hours.

In certain embodiments, the present invention is directed to the above formulations and methods, wherein the mean Tmax of dexamethasone in the plasma occurs at a time point from about 12 to about 30 hours.

In certain embodiments, the present invention is directed to the above formulations and methods, wherein the mean AUCt of bupivicaine at 96 hours in the plasma is below about 12,000 ng/ml*h.

In certain embodiments, the present invention is directed to the above formulations and methods, wherein the mean AUC of dexamethasone at 96 hours in the plasma is below about 15 ng/ml*h.

In certain embodiments, the present invention is directed to the above formulations and methods, wherein the formulation provides an effect characterized by a mean pin prick pain response test which is less than 1.0 at 3 hours after administration; less than 1.0 at 24 hours after administration; less than 1.0 at 48 hours after administration; less than 1.0 at 72 hours after administration; or less than 1.0 at 96 hours after administration. In certain embodiments, the invention is directed to methods and formulations which provide the above pin prick test results at more than one or all of the above time points.

In certain embodiments, the present invention is directed to the above formulations and methods, wherein the formulation provides an effect characterized by a mean somesthetic response test which is less than 0.6 at 3 hours after administration; less than 0.6 at 24 hours after administration; less than 0.6 at 48 hours after administration; less than 0.6 at 72 hours after administration; or less than 0.6 at 96 hours after administration. In certain embodiments, the invention is directed to methods and formulations which provide the above somesthetic response test results at more than one or all of the above time points.

In certain embodiments, the present invention is directed to the above formulations and methods, wherein the formulation provides an effect characterized by a mean warmth detection threshold result which is at least 3 degrees C. over the baseline at 3 hours after administration; at least 3 degrees C. over the baseline at 24 hours after administration; at least 3 degrees C. over the baseline at 48 hours after administration; at least 3 degrees C. over the baseline at 72 hours after administration; or at least 3 degrees C. over the baseline at 96 hours after administration. In certain embodiments, the invention is directed to methods and formulations which provide the above mean warmth detection threshold results at more than one or all of the above time points.

In certain embodiments, the present invention is directed to the above formulations and methods, wherein the formulation provides an effect characterized by a mean heat pain detection threshold result which is at least 3 degrees C. over the baseline at 3 hours after administration; at least 3 degrees C. over the baseline at 24 hours after administration; at least 3 degrees C. over the baseline at 48 hours after administration; or at least 3 degrees C. over the baseline at 72 hours after administration. In certain embodiments, the invention is directed to methods and formulations which provide the above mean heat pain detection threshold results at more than one or all of the above time points.

The certain embodiments, the present invention is directed to methods of preparing the formulations disclosed herein.

In certain embodiments, the invention is directed to a method of detecting the local concentration of a local anesthetic at a site of administration comprising administering a local anesthetic at a site of a human and measuring the concentration of said local anesthetic in the tissue of said site by microdialysis at one or more time intervals.

In certain embodiments, the invention is directed to a method of detecting the local concentration of a corticosteroid at a site of administration comprising administering a corticosteroid at a site of a human and measuring the concentration of said local anesthetic in the tissue of said site by microdialysis at one or more time intervals.

In one embodiment, the invention is directed to a formulation for providing local analgesia, local anesthesia or nerve blockade in a human, comprising a biocompatible, biodegradable carrier including a local anesthetic, said formulation providing local analgesia, local anesthesia or nerve blockade at the site of administration in a human which, upon first administration, occurs less than about 2 hours after administration, and a duration of local analgesia, local anesthesia or nerve blockade which lasts for at least about 2 days after administration, wherein the level of local anesthetic in blood plasma after administration does not reach toxic levels.

Embodiment above, wherein said formulation further comprises an augmenting agent in an amount effective to prolong the effect of the local anesthetic for a time period greater than that obtained by use of the local anesthetic in controlled release form alone, said formulation having a duration of local analgesia which lasts for at least about 4 days after administration.

Embodiments above, wherein the duration of local analgesia is from about 2 to about 4 days after administration.

Embodiments above wherein the duration of local analgesia is from about 4 to about 7 days after administration.

Embodiments above which further comprises a dose of a second local anesthetic in immediate release form, said second local anesthetic providing said formulation with an onset of activity not more than about 5 minutes after administration of the formulation.

Embodiments above, wherein said carrier comprises microspheres comprising said local anesthetic and a biocompatible, biodegradable polymer.

Any of the foregoing embodiments, where the local anesthetic is bupivacaine free base.

Any of the foregoing embodiments, where the effect lasts for at least about 2 days.

Any of the foregoing embodiments, where said formulation further includes an effective amount of an augmenting agent selected from the group consisting of a glucocorticosteroid, a neurosteroid, a vasoconstricting agent, a modulator of ionic transport across cell membranes, a tubulin binding agent, a sodium/potassium ATP-ase inhibitor, and combinations of any of the foregoing.

Any of the foregoing embodiments, where the polymer is a 65:35 DL copolymer of lactic and glycolic acid having an inherent viscosity from about 0.25 to about 0.42 dL/g, a molecular weight of about 40 kDa, and free carboxylic acid end groups.

Any of the foregoing embodiments, where the local anesthetic is bupivacaine free base, the augmenting agent is dexamethasone, and the polymer is a copolymer of lactic and glycolic acid.

Any of the foregoing embodiments, where the carrier comprises microspheres comprising a polymer selected the group consisting of polyanhydrides, polyesters, copolymers of lactic acid and glycolic acid, polyorthoesters, proteins, and polysaccharides.

Any of the foregoing embodiments, where carrier further comprises a glucocorticosteroid incorporated at a loading between about 0.001 and about 30 percent by weight.

Embodiments above, where the glucocorticosteroid is dexamethasone.

Any of the foregoing embodiments, where the local anesthetic is incorporated into the controlled release form at a percent loading of ranging from about 60% to about 85% by weight.

Any of the foregoing embodiments, where the formulation comprises a plurality of microcapsules.

Any of the foregoing embodiments, where the carrier is suspended in a pharmaceutically acceptable vehicle for injection.

A formulation for providing local analgesia in a human, comprising a plurality of controlled release microspheres comprising bupivacaine free base and a biocompatible, biodegradable polymer comprising a 65:35 DL copolymer of lactic and glycolic acid having an inherent viscosity from about 0.25 to about 0.42 dL/g, a molecular weight of about 40 kDa, and free carboxylic acid end groups, said bupivacaine free base being contained in said microspheres at a drug loading of from about 60% to about 85%, by weight, said microspheres being contained in a pharmaceutically acceptable medium for parenteral administration at a concentration sufficient to provide a concentration of bupivacaine free base from about 2.25 mg/ml to about 36.0 mg/ml and providing a unit dose of bupivacaine free base from about 45 mg to about 360 mg, said formulation providing an onset of local analgesia at the site of administration which occurs less than about 2 hours after administration, and a duration of local analgesia which lasts for at least about 1 day after administration.

Embodiments above where the microspheres are contained in the medium at a concentration of about 6.25 mg/ml with about 16 ml of said medium at a strength of about 4.5 mg/ml of bupivacaine.

Embodiments above where the microspheres further comprise dexamethasone, and said formulation includes about 2.5 mcg/ml dexamethasone.

Embodiments above where the microspheres are contained in the medium at a concentration of about 12.5 mg/ml with about 8 ml of said medium at a strength of about 9 mg/ml bupivacaine.

Embodiments above where the microspheres further comprise dexamethasone, and said formulation includes about 5.0 mcg/ml dexamethasone.

Embodiments above where the microspheres contained in the medium at a concentration of about 25.0 mg/ml with about 4 ml of said medium at a strength of about 18 mg/ml bupivacaine.

Embodiments above where the microspheres further comprise dexamethasone, and said formulation includes about 10.0 mcg/ml dexamethasone.

Embodiments above where the microspheres are contained in the medium at a concentration of about 3.125 mg/ml with about 16 ml of said medium at a strength of about 2.25 mg/ml of bupivacaine and about 1.25 mcg/ml dexamethasone.

Any of the foregoing embodiments where the polymer is a copolymer of lactic and glycolic acid that is terminated with free carboxylic acid end groups.

Any of the foregoing embodiments, where the carrier is a 65:35 DL copolymer of lactic and glycolic acid having an inherent viscosity from about 0.25 to about 0.42 dL/g and a molecular weight of from about 10 kDa to about 150 kDa.

Any of the foregoing embodiments, where the carrier is a 65:35 DL copolymer of lactic and glycolic acid having an inherent viscosity from about 0.2 to about 0.6 dL/g and a molecular weight of from about 20 kDa to about 80 kDa.

Any of the foregoing embodiments, where the carrier is a 65:35 DL copolymer of lactic and glycolic acid having an inherent viscosity from about 0.7 to about 1.0 dL/g and a molecular weight of from about 100 kDa to about 150 kDa.

Any of the foregoing embodiments, where the carrier is a 65:35 DL copolymer of lactic and glycolic acid having an inherent viscosity from about 0.25 to about 0.42 dL/g and a molecular weight of from about 40 kDa to about 120 kDa.

A formulation for providing local analgesia, local anesthesia or nerve blockade in a human, comprising a biocompatible, biodegradable carrier including a local anesthetic, said formulation providing local analgesia, local anesthesia or nerve blockade at the site of administration in a human which, upon first administration, occurs less than about 2 hours after administration, and a duration of local analgesia, local anesthesia or nerve blockade which lasts for at least about 2 days after administration, wherein the level of local anesthetic in blood plasma after administration does not reach toxic levels, which formulation provides an in-vitro dissolution of the local anesthetic from the biocompatible, biodegradable carrier under in-vitro conditions specified by the USP II Paddle Method, 100 RPM, 37 degrees Celcius, pH 3.0 in 900 ml of 10 mM sodium phosphate buffer, as follows:

| TIME (Hours) | Percent Release |
|---|---|
| 0 | 0 |
| 0.25 | about 2 to about 32 |
| 0.5 | about 3 to about 60 |
| 1 | about 6 to about 86 |
| 1.5 | about 9 to about 92 |
| 2 | about 12 to about 94 |
| 3 | about 17 to about 97 |
| 4 | about 23 to about 97 |

Any of the foregoing embodiments, where the formulation is further assessed in the rat using hotplate model and provides a mean latency greater than about 2 seconds to about 12 seconds.

Any of the foregoing embodiments, where the formulation is further assessed in the rat using hotplate model and provides a mean latency greater than about 7 seconds to about 12 seconds.

Embodiments above where at least 50% of the rats tested experience the stated latency range.

Any of the foregoing embodiments, where the formulation provides an in-vitro dissolution of the local anesthetic from the biocompatible, biodegradable carrier under in-vitro conditions specified by the USP II Paddle Method, 100 RPM, 37 degrees Celcius, pH 3.0 in 900 ml of 10 mM sodium phosphate buffer, as follows:

| TIME (Hours) | Percent Release |
|---|---|
| 0 | 0 |
| 1 | From about 13 to about 36 |
| 2 | From about 33 to about 65 |
| 4 | From about 53 to about 87 |
| 8 | From about 72 to about 95 |
| 12 | From about 81 to about 98 |
| 18 | From about 89 to about 100 |
| 24 | From about 94 to about 100 |

A method for providing prolonged local analgesia at a site in a human, comprising administering a formulation comprising a local anesthetic in a biocompatible, biodegradable carrier including a local anesthetic, said formulation being capable of parenteral administration, said formulation providing an onset of local anesthesia or pain relief or nerve blockage at the site of administration in a human which, upon first administration, occurs less than about 2 hours after administration, and a duration of local analgesia which lasts for a time period of at least about 2 days after administration.

Embodiments of the Invention: Parenteral Administration

Any of the foregoing embodiments, which provide an effect characterized by the lowest force or number of a von Frey hair which produces a sensation of pain in a mechanical pain detection threshold test in a human patient, as follows: from about 13 to about 18 at 2 hours after administration; from about 13 to about 18 at 4 hours after administration; from about 14 to about 18 at 8 hours after administration; from about 13 to about 18 at 24 hours after administration; from about 13 to about 18 at 48 hours after administration; from about 13 to about 18 at 72 hours after administration; from about 12 to about 18 at 96 hours after administration; from about 11 to about 18 at 144 hours after administration, from about 15 to about 18 at 168 hours after administration, and from about 15 to about 18 at 192 hours after administration, based on a baseline test from about 13 to about 17, when the formulation is parenterally administered.

Any of the foregoing embodiments, which provide an effect characterized by the lowest force or number of a von Frey hair which produces a sensation of pain in a mechanical pain detection threshold test in a human patient, as follows: at least about 13 at 2 hours after administration; at least about 13 at 4 hours after administration; at least about 14 at 8 hours after administration; at least about 13 at 24 hours after administration; at least about 13 at 48 hours after administration; at least about 13 at 72 hours after administration; at least about 12 at 96 hours after administration; at least about 12 at 144 hours after administration, at least about 12 at 168 hours after administration, and at least about 12 to at 192 hours after administration, based on a baseline of a minimum von Frey hair number of about 10 and a maximum possible von Frey hair number of 18, when the formulation is administered parenterally.

Any of the foregoing embodiments, which provide a median effect across a patient population characterized by the lowest force or number of a von Frey hair which produces a sensation of pain in a mechanical pain detection threshold test in a human patient, as follows: about 16 to about 17 at 2 hours after administration; from about 16 to about 17 at 4 hours after administration; about 18 at 8 hours after administration; from about 17.5 to about 18 at 24 hours after administration; from about 17 to about 18 at 48 hours after administration; from about 16 to about 18 at 72 hours after administration; from about 15 to about 16.5 at 96 hours after administration; and from about 15 to about 16 at 144 hours after administration, based on a baseline test of about 15, when the formulation is administered parenterally.

Any of the foregoing embodiments, which provide a median effect across a patient population characterized by the lowest force or number of a von Frey hair which produces a sensation of pain in a mechanical pain detection threshold test in a human patient, as follows: about 13.5 to about 17.5 at 2 hours after administration; from about 11.5 to about 18 at 4 hours after administration; from about 11.5 to about 18 at 8 hours after administration; from about 13 to about 18 at 24 hours after administration; from about 15 to about 18 at 48 hours after administration; from about 15.5 to about 18 at 72 hours after administration; from about 15 to about 18 at 96 hours after administration; and from about 15 to about 16 at 144 hours after administration, based on a baseline test of about 15, when the formulation is administered parenterally.

Any of the foregoing embodiments, which provide an effect characterized by a mechanical pain detection threshold test in human patients in which the median lowest number of the von Frey hair in which half of the stimulations produces a sensation of pain or unpleasantness is as follows: from about 16 to about 17 at 2 hours after administration; from about 16 to about 17 at 4 hours after administration; about 18 at 8 hours after administration; from about 17.5 to about 18 at 24 hours after administration; from about 17 to about 18 at 48 hours after administration; from about 16 to about 18 at 72 hours after administration; from about 15 to about 16.5 at 96 hours after administration; and from about 15 to about 16 at 144 hours after administration, based on a median baseline test of about 15, when the formulation is administered parenterally.

Any of the foregoing embodiments, which provide an effect characterized by a mechanical pain detection threshold test in human patients in which the median lowest number of the von Frey hair in which half of the stimulations produces a sensation of pain or unpleasantness is as follows: from about 16 to about 17 at 2 hours after administration; from about 16 to about 17 at 4 hours after administration; about 18 at 8 hours after administration; from about 17.5 to about 18 at 24 hours after administration; from about 17 to about 18 at 48 hours after administration; from about 16 to about 18 at 72 hours after administration; from about 15 to about 16.5 at 96 hours after administration; and from about 15 to about 16 at 144 hours after administration, based on a median baseline test of about 15, when the formulation is administered parenterally.

Any of the foregoing embodiments, which provide an effect characterized by a mechanical pain detection threshold test in human patients in which the median lowest number of the von Frey hair in which half of the stimulations produces a sensation of pain or unpleasantness is as follows: about 17 at 2 hours after administration; about 17 at 4 hours after administration; about 18 at 8 hours after administration; about 18 at 24 hours after administration; about 18 at 48 hours after administration; about 18 at 72 hours after administration; and about 16.5 at 96 hours after administration, when the formulation is administered parenterally.

Any of the foregoing embodiments, which provide an effect characterized by a mechanical pain detection threshold test in human patients in which the median lowest number of the von Frey hair in which half of the stimulations produces a sensation of pain or unpleasantness is as follows: about 13.5 to about 17.5 at 2 hours after administration; from about 11.5 to about 18 at 4 hours after administration; from about 11.5 to about 18 at 8 hours after administration; from about 13 to about 18 at 24 hours after administration; from about 15 to about 18 at 48 hours after administration; from about 15.5 to about 18 at 72 hours after administration; from about 15 to about 18 at 96 hours after administration; and from about 15 to about 16 at 144 hours after administration, based on a baseline test of about 15, when the formulation is administered parenterally.

Any of the foregoing embodiments, which provide a mechanical pain detection threshold of about 16 at 144 hours after administration.

Any of the foregoing embodiments, wherein the median baseline mechanical pain detection threshold is from about 14.5 to about 16.5.

Any of the foregoing embodiments, which provide an effect characterized by a mechanical pain detection threshold test in human patients in which the median lowest number of the von Frey hair in which half of the stimulations produces a sensation of pain or unpleasantness is as follows: about 16 at 2 hours after administration; about 16 at 4 hours after administration; about 18 at 8 hours after administration; about 17.5 at 24 hours after administration; and about 17 at 48 hours after administration, based on a baseline test of about 15.

Any of the foregoing embodiments, which provide an effect characterized by a mechanical pain detection threshold of about 16 at 72 hours after administration.

A formulation for providing local analgesia in a human, comprising a biocompatible, biodegradable carrier including a local anesthetic, said formulation being capable of parenteral administration, said formulation providing an effect characterized by a mechanical pain detection threshold test in human patients in which the lowest number of the von Frey hair in which half of the stimulations produces a sensation of pain or unpleasantness is from about 16 to about 18 from about 2 to at least about 48 hours after administration, where the median baseline test is about 15, when the formulation is administered via perineurial, subcutaneous or intramuscular administration.

Any of the embodiments set forth above, wherein the lowest number of the von Frey hair in which half of the stimulations produced a sensation of pain or unpleasantness is from about 16 to about 18 from about 2 to at least about 72 hours after administration.

Any of the embodiments set forth above, wherein the lowest number of the von Frey hair in which half of the stimulations produced a sensation of pain or unpleasantness is at least 16 from about 2 to at least about 96 hours after administration.

Any of the embodiments set forth above, wherein the lowest number of the von Frey hair in which half of the stimulations produced a sensation of pain or unpleasantness is at least about 16 from about 2 hours to at least 5 days after administration.

Any of the embodiments set forth above, which provide an effect characterized by a mechanical pain detection threshold test in human patients in which the mean lowest number of the von Frey hair in which half of the stimulations produces a sensation of pain or unpleasantness is as follows: from about 15.6 to about 16.9 at 2 hours after administration; from about 15.7 to about 17.3 at 4 hours after administration; form about 16.4 to about 17.7 at 8 hours after administration; from about 16.2 to about 18 at 24 hours after administration; from about 15.7 to about 17.8 at 48 hours after administration; from about 15.5 to about 17.5 at 72 hours after administration; from about 15.1 to about 16.9 at 96 hours after administration; and from about 15.1 to about 16.8 at 144 hours after administration, when the formulation is administered via perineurial, subcutaneous or intramuscular administration.

Any of the embodiments set forth above, which provide an effect characterized by a mechanical pain detection threshold test in human patients in which the mean lowest number of the von Frey hair in which half of the stimulations produces a sensation of pain or unpleasantness is as follows: from about 13 to about 17.7 at 2 hours after administration; from about 11 to about 18 at 4 hours after administration; form about 11 to about 18 at 8 hours after administration; from about 13 to about 18 at 24 hours after administration; from about 14 to about 18 at 48 hours after administration; from about 14 to about 18 at 72 hours after administration; from about 15 to about 18.4 at 96 hours after administration; and at least about 15 for at least about 144 hours after administration, when the formulation is administered via perineurial, subcutaneous or intramuscular administration.

Any of the embodiments set forth above, which provide an effect characterized by a mechanical pain detection threshold test in human patients in which the mean lowest number of the von Frey hair in which half of the stimulations produces a sensation of pain or unpleasantness is as follows: about 16.46±0.39 at 2 hours after administration; about 16.85±0.42 at 4 hours after administration; about 17.38±0.31 at 8 hours after administration; about 17.92±0.08 at 24 hours after administration; about 17.33±0.47 at 48 hours after administration; about 17.0±0.54 at 72 hours after administration; and about 16.33±0.54 at 96 hours after administration.

Any of the embodiments set forth above, which provide a mechanical pain detection threshold of about 16.17±0.6 at 144 hours after administration.

Any of the embodiments set forth above, wherein the mean baseline mechanical pain detection threshold is about 15.38±0.27.

Any of the embodiments set forth above, which provide an effect characterized by a mechanical pain detection threshold test in human patients in which the mean lowest number of the von Frey hair in which half of the stimulations produces a sensation of pain or unpleasantness is as follows: about 16.08±0.49 at 2 hours after administration; about 16.23 ±0.53 at 4 hours after administration; about 16.85±0.44 at 8 hours after administration; about 16.75±0.51 at 24 hours after administration; and about 16.25±0.57 at 48 hours after administration, based on a baseline of about 15.31±33.

Any of the embodiments set forth above, which provide an effect characterized by a mechanical pain detection threshold of about 16.08±0.54 at 72 hours after administration.

A formulation for providing local analgesia in a human, comprising a biocompatible, biodegradable carrier including a local anesthetic, said formulation being capable of parenteral administration, said formulation providing an effect characterized by a mechanical pain detection threshold test in human patients in which the mean lowest number of the von Frey hair in which half of the stimulations produced a sensation of pain or unpleasantness is from about 15.1 to about 18 from about 2 to at least about 96 hours after administration, when the formulation is administered parenterally.

Any of the embodiments set forth above, wherein the mean lowest number of the von Frey hair in which half of the stimulations produced a sensation of pain or unpleasantness is from about 15.7 to about 17.8 from about 2 to at least about 48 hours after administration.

Any of the embodiments set forth above, which provide an effect characterized by a mechanical touch detection threshold test in which the lowest force or number of a von Frey hair which produces a sensation of touch or pressure in a human patient is as follows: from about 8 to about 15 at 2 hours after administration; from about 9 to about 18 at 4 hours after administration; from about 9 to about 18 at 8 hours after administration; from about 9 to about 18 at 24 hours after administration; from about 9 to about 18 at 48 hours after administration; from about 9 to about 15 at 72 hours after administration; from about 9 to about 14 at 96 hours after administration; and from about 9 to about 14 at 144 hours after administration, when the formulation is administered via perineurial, subcutaneous or intramuscular administration.

Any of the embodiments set forth above, which provide an effect characterized by a mechanical touch detection threshold test in which the lowest force or number of a von Frey hair which produces a sensation of touch or pressure in a human patient is as follows: from about 4 to about 15 at 2 hours after administration; from about 4 to about 18 at 4 hours after administration; from about 5 to about 18 at 8 hours after administration; from about 3 to about 18 at 24 hours after administration; from about 4 to about 16 at 48 hours after administration; from about 4 to about 18 at 72 hours after administration; and at least about 3 to about 18 for at least 96 hours after administration; when the formulation is administered via perineurial, subcutaneous or intramuscular administration.

Any of the embodiments set forth above, which provide an effect characterized by a mechanical touch detection threshold test in which the median lowest force or number of a von Frey hair which produces a sensation of touch or pressure in human patients is as follows: about 11 at 2 hours after administration; from about 11 to about 12 at 4 hours after administration; from about 12 to about 14 at 8 hours after administration; from about 13 to about 14 at 24 hours after administration; from about 11 to about 13 at 48 hours after administration; from about 10 to about 11.5 at 72 hours after administration; from about 10.5 to about 11 at 96 hours after administration; and from about 10 to about 11.5 at 144 hours after administration, based on a median baseline test of about 9, when the formulation is administered via perineurial, subcutaneous or intramuscular administration.

Any of the embodiments set forth above, which provide an effect characterized by a mechanical touch detection threshold test in which the median lowest force or number of a von Frey hair which produces a sensation of touch or pressure in human patients is as follows: about 11 at 2 hours after administration; about 12 at 4 hours after administration; about 14 at 8 hours after administration; about 14 at 24 hours after administration; about 13 at 48 hours after administration; about 11.5 at 72 hours after administration; about 11 at 96 hours after administration; and about 11.5 at 144 hours after administration, based on a median baseline test of about 9, when the formulation is administered via perineurial, subcutaneous or intramuscular administration.

Any of the embodiments set forth above, which provide an effect characterized by a mechanical touch detection threshold test in which the median lowest force or number of a von Frey hair which produces a sensation of touch or pressure in human patients is as follows: about 11 at 2 hours after administration; about 11 at 4 hours after administration; about 12 at 8 hours after administration; about 13 at 24 hours after administration; about 11 at 48 hours after administration, based on a median baseline test of about 9.

Any of the embodiments set forth above, which provide an effect further characterized by a mechanical touch detection threshold test in human patients in which the median lowest force or number of a von Frey hair which produces a sensation of touch or pressure in a human patient as follows: about 10 at 72 hours after administration.

A formulation for providing local analgesia in a human, comprising a biocompatible, biodegradable carrier including a local anesthetic, said formulation being capable of parenteral administration, said formulation providing an effect characterized by a mechanical touch detection threshold test in human patients in which the median lowest force or number of a von Frey hair which produces a sensation of touch or pressure in human patients is from about 11 to about 14 from about 2 to at least about 96 hours after administration, where the median baseline test is about 9, when the formulation is administered via perineurial, subcutaneous or intramuscular administration.

Any of the embodiments set forth above, wherein the median lowest number of the von Frey hair in which half of the stimulations produced a sensation of pain or unpleasantness is from about 11 to about 14 from about 2 to at least about 144 hours after administration.

A formulation for providing local analgesia in a human, comprising a biocompatible, biodegradable carrier including a local anesthetic, said formulation being capable of parenteral administration, said formulation providing an effect characterized by a mechanical touch detection threshold test in human patients in which the median lowest force or number of a von Frey hair which produces a sensation of touch or pressure in human patients is from about 11 to about 13 from about 2 to at least about 48 hours after administration, where the median baseline test is about 9, when the formulation is administered via perineurial, subcutaneous or intramuscular administration.

Any of the embodiments set forth above, wherein the median lowest number of the von Frey hair in which half of the stimulations produced a sensation of pain or unpleasantness is from about 10 to about 13 from about 2 to at least about 72 hours after administration.

Any of the embodiments set forth above, which provide an effect characterized by a mechanical touch detection threshold test in which the mean lowest force or number of a von Frey hair which produces a sensation of touch or pressure in human patients is as follows: from about 10.4 to about 11.7 at 2 hours after administration; from about 11.0 to about 12.5 at 4 hours after administration; from about 12.1 to about 14.0 at 8 hours after administration; from about 12.0 to about 15.0 at 24 hours after administration; from about 10.8 to about 14.0 at 48 hours after administration; from about 9.9 to about 12.4 at 72 hours after administration; from about 10.1 to about 11.7 at 96 hours after administration; and from about 9.8 to about 11.7 at 144 hours after administration, based on a mean baseline test from about 8.8 to about 9.2, when the formulation is administered via perineurial, subcutaneous or intramuscular administration.

Any of the embodiments set forth above, which provide an effect characterized by a mechanical touch detection threshold test in which the mean lowest force or number of a von Frey hair which produces a sensation of touch or pressure in human patients is as follows: from about 5 to about 12.09 at 2 hours after administration; from about 4 to about 13.5 at 4 hours after administration; from about 5 to about 15 at 8 hours after administration; from about 5 to about 15.6 at 24 hours after administration; from about 5 to about 16.2 at 48 hours after administration; from about 5 to about 16.2 at 72 hours after administration; from about 3 to about 15.2 at 96 hours, based on a mean baseline test from about 5 to about 9.9, when the formulation is administered via perineurial, subcutaneous or intramuscular administration.

Any of the embodiments set forth above, which provide an effect characterized by a mechanical touch detection threshold test in which the mean lowest force or number of a von Frey hair which produces a sensation of touch or pressure in human patients is as follows: about 11.08±0.64 at 2 hours after administration; about 11.77±0.72 at 4 hours after administration; about 13.15±0.82 at 8 hours after administration; about 14.08±0.88 at 24 hours after administration; about 13.5±0.53 at 48 hours after administration; about 12±0.41 at 72 hours after administration; and about 11.25±0.46 at 96 hours after administration, based on a mean baseline mechanical pain detection threshold of 9.1±0.23, based on a baseline of about 9.0±0.23.

Any of the embodiments set forth above, which provide a mean mechanical touch detection threshold of about 11.33±0.38 at 120 hours after administration.

Any of the embodiments set forth above, which provide an effect characterized by a mechanical touch detection threshold test in which the mean lowest force or number of a von Frey hair which produces a sensation of touch or pressure in human patients is as follows: about 10.92±0.57 at 2 hours after administration; about 11.69±0.67 at 4 hours after administration; about 12.85±0.74 at 8 hours after administration; about 12.83±0.84 at 24 hours after administration; and about 11.67±0.9 at 48 hours after administration.

Any of the embodiments set forth above, which provide an effect further characterized by a mean mechanical touch detection threshold of about 10.42±0.48 at 72 hours after administration.

Any of the embodiments set forth above, wherein the mean baseline mechanical pain detection threshold is about 8.85±0.1.

A formulation for providing local analgesia in a human, comprising a biocompatible, biodegradable carrier including a local anesthetic, said formulation being capable of parenteral administration, said formulation providing an effect characterized by a mechanical touch detection threshold test in human patients in which the mean lowest force or number of a von Frey hair which produces a sensation of touch or pressure in human patients is from about 10.4 to about 15 from about 2 to at least about 96 hours after administration, based on a mean baseline test from about 8.8 to about 9.2, when the formulation is administered via perineurial, subcutaneous or intramuscular administration.

Any of the embodiments set forth above, wherein the mean lowest number of the von Frey hair in which half of the stimulations produced a sensation of pain or unpleasantness is from about 10.4 to about 15 from about 2 to at least about 144 hours after administration.

A formulation for providing local analgesia in a human, comprising a biocompatible, biodegradable carrier including a local anesthetic, said formulation being capable of parenteral administration, said formulation providing an effect characterized by a mechanical touch detection threshold test in human patients in which the mean lowest force or number of a von Frey hair which produces a sensation of touch or pressure in human patients is from about 10.4 to about 13.7 from about 2 to at least about 48 hours after administration, where the mean baseline test is from about 8.8 to about 9.0, when the formulation is administered via perineurial, subcutaneous or intramuscular administration.

Any of the embodiments set forth above, wherein the mean lowest number of the von Frey hair in which half of the stimulations produced a sensation of pain or unpleasantness is from about 9.9 to about 13.7 from about 2 to at least about 72 hours after administration.

Any of the embodiments set forth above, which provide an effect characterized by a warm detection threshold test in which the median lowest increase in temperature from 32 C perceived by human patients, occurs at a temperature as follows in degrees C.: about 40.5 to about 44.05 at 2 hours after administration; about 40.15 to about 44.85at 4 hours after administration; about 40.15 to about 46.3 at 8 hours after administration; from about 41.7 to about 46.35 at 24 hours after administration; about 41.55 at 48 hours after administration; from about 40.4 to about 46.55 at 72 hours after administration; from about 41.1 to about 45.7 at 96 hours after administration; based on a median baseline test from about 39.9 to about 41.95, when the formulation is administered via perineurial, subcutaneous or intramuscular administration.

A formulation for providing local analgesia in a human, comprising a biocompatible, biodegradable carrier including a local anesthetic, said formulation being capable of parenteral administration, said formulation providing an effect characterized by a warm detection threshold test in which the median lowest increase in temperature from 32 C perceived by human patients, is from about 43 to about 46.9 from a time of about 2 to at least about 48 hours after administration, based on a median baseline test from about 41.6 to about 42.6, when the formulation is administered via perineurial, subcutaneous or intramuscular administration.

Any of the embodiments set forth above, which provide an effect characterized by a warm detection threshold test in which the mean lowest increase in temperature from 32 C perceived by human patients, occurs at a temperature as follows in degrees C.: about 43.2 to about 46.5 at 2 hours after administration; from about 44.1 to about 46.2 at 4 hours after administration; from about 44.8 to about 46.9 at 8 hours after administration; from about 45.6 to about 46.9 at 24 hours after administration; from about 44.1 to about 46.9 at 48 hours after administration; from about 42.6 to about 45.9 at 72 hours after administration; from about 41.5 to about 44.9 at 96 hours after administration; and from about 42.0 to about 43.5 at 144 hours after administration, based on a mean baseline test from about 41.1 to about 42.5, when the formulation is administered via perineurial, subcutaneous or intramuscular administration.

Any of the embodiments set forth above, which provide an effect characterized by a warm detection threshold test in which the mean lowest increase in temperature from 32 C perceived by human patients, occurs at a temperature as follows in degrees C.: about 40.2 to about 44.7 at 2 hours after administration; from about 40.3 to about 45.6 at 4 hours after administration; from about 39 to about 46.4 at 8 hours after administration; from about 40.1 to about 47.2 at 24 hours after administration; from about 39.1 to about 47.2 at 48 hours after administration; from about 39 to about 46.9 at 72 hours after administration; from about 39.7 to about 46.2 at 96 hours after administration;, based on a mean baseline test from about 39 to about 44.08, when the formulation is administered via perineurial, subcutaneous or intramuscular administration.

Any of the embodiments set forth above, which provides an effect characterized by a warm detection threshold test in which the mean lowest increase in temperature from 32 C perceived by human patients, occurs at a temperature as follows in degrees C.: about 43.82±0.65 at 2 hours after administration; about 44.69±0.64 at 4 hours after administration; about 45.35±0.56 at 8 hours after administration; about 46.39±0.54 at 24 hours after administration; about 46.09±0.76 at 48 hours after administration; about 45.19±0.67 at 72 hours after administration; and about 44.19±0.7 at 96 hours after administration, based on a mean baseline warm detection threshold of about 41.97±0.56.

Any of the embodiments set forth above, which provides a mean warm detection threshold of about 43.01±0.5 at 144 hours after administration.

Any of the embodiments set forth above, which provides an effect characterized by a warm detection threshold test in which the mean lowest increase in temperature from 32 C. perceived by human patients, occurs at a temperature as follows in degrees C.: about 45.72±0.76 at 2 hours after administration; about 45.42±0.78 at 4 hours after administration; about 46.22±0.65 at 8 hours after administration; and about 46.11±0.49 at 24 hours after administration; and about 44.72±0.65 at 48 hours after administration, based on a baseline test of about 41.64±0.54.

Any of the embodiments set forth above, which provide an effect further characterized by a mean warm detection threshold of about 42.97±0.4 at 72 hours after administration.

A formulation for providing local analgesia and/or anesthesia in a human, comprising a biocompatible, biodegradable carrier including a local anesthetic, said formulation being capable of subcutaneous administration, said formulation providing an effect characterized by a warm detection threshold test in which the mean lowest increase in temperature from 32 C perceived by human patients occurs at from about 41.5 C to about 46.9 C from about 2 to at least about 96 hours after administration, where the mean baseline test is from about 41.1 to about 42.5, when the formulation is administered via via perineurial, subcutaneous or intramuscular administration.

Any of the embodiments set forth above, which provide an effect wherein pinpricks are perceived as touch or pressure, having an onset of at least 0.5 hours and a duration of at least about 14 hours.

Any of the embodiments set forth above, wherein the effect lasts for about 110 hours.

Any of the embodiments set forth above, which provide a tactile perception block having an onset of at least about 1 hour and a duration of at least about 3 hours.

Any of the embodiments set forth above, which provide an effect wherein a temperature of 52° C. is not perceived as painful, having an onset of at least about 1 hour and a duration of at least about 2 days

Any of the embodiments set forth above, wherein the effect lasts for at least about 2 days.

Any of the embodiments set forth above, which provide an effect characterized by perception of a temperature as painful, said temperature being at least 3° C. greater than the temperature that is perceived as painful prior to administration of the formulation, having an onset of at least about 1 hour and a duration of at least about 2 days.

Any of the embodiments set forth above, wherein the duration is at least about 4 days.

Any of the embodiments set forth above, which provide an effect wherein a temperature of 52° C. is not perceived as warm, having an onset of at least about 1 hour and a duration of about 4 days hours.

Any of the embodiments set forth above, which provide an effect characterized by a heat pain detection threshold test in which the median lowest temperature above 32 C perceived as painful by human patients is as follows in degrees C.: about 49.1 to about 50.2 at 2 hours after administration; from about 49.9 to about 50.9 at 4 hours after administration; about 50.9 to about 51 at 8 hours after administration; from about 50.4 to about 50.75 at 24 hours after administration; from about 50.1 to about 51.05 at 48 hours after administration; from about 49.4 to about 50.65 at 72 hours after administration; from about 49.05 to about 50.3 at 96 hours after administration; and from about 49.4 to about 50.4 at 144 hours after administration, based on a median baseline test from about 48.9 to about 49.1, when the formulation is administered via perineurial, subcutaneous or intramuscular administration.

Any of the embodiments set forth above, which provide an effect characterized by a heat pain detection threshold test in which the median lowest temperature above 32 C perceived as painful by human patients is as follows in degrees C.: about 47.15 to about 49.2 at 2 hours after administration; from about 47.05 to about 50.3 at 4 hours after administration; about 47.3 to about 50.35 at 8 hours after administration; from about 47.3 to about 51.7 at 24 hours after administration; from about 47.75 to about 51.85 at 48 hours after administration; from about 46.85 to about 50.95 at 72 hours after administration; from about 47.45 to about 51.2 at 96 hours after administration; based on a median baseline test from about 46.8 to about 49, when the formulation is administered via perineurial, subcutaneous or intramuscular administration.

Any of the embodiments set forth above, which provide an effect characterized by a heat pain detection threshold test in which the mean lowest temperature above 32 C perceived as painful by human patients is as follows in degrees C.: about 48.8 to about 50.2 at 2 hours after administration; from about 49.2 to about 50.9 at 4 hours after administration; about 49.8 to about 50.9 at 8 hours after administration; from about 50.5 to about 51.6 at 24 hours after administration; from about 49.4 to about 51.8 at 48 hours after administration; from about 48.6 to about 51.2 at 72 hours after administration; from about 47.9 to about 51.1 at 96 hours after administration; and from about 48.9 to about 50.5 at 144 hours after administration, based on a mean baseline test from about 47.9 to about 49.2, when the formulation is administered via perineurial, subcutaneous or intramuscular administration.

Any of the embodiments set forth above, which provide an effect characterized by a heat pain detection threshold test in which the mean lowest temperature above 32 C perceived as painful by human patients is as follows in degrees C.: about 49.25±0.42 at 2 hours after administration; about 49.65±0.41 at 4 hours after administration; about 50.15±0.36 at 8 hours after administration; about 51.09±0.46 at 24 hours after administration; about 51.17±0.64 at 48 hours after administration; about 50.73±0.48 at 72 hours after administration; and about 50.7±0.43 at 96 hours after administration, based on a mean baseline heat pain detection threshold of about 48.52±0.59, when the formulation is administered via perineurial, subcutaneous or intramuscular administration.

Any of the embodiments set forth above, which provide a mean heat pain detection threshold of about 49.93±0.52 at 144 hours after administration.

Any of the embodiments set forth above, which provide an effect characterized by a heat pain detection threshold test in which the mean lowest temperature above 32 C perceived as painful by human patients is as follows in degrees C.: about 49.54±0.63 at 2 hours after administration; about 50.38±0.53 at 4 hours after administration; about 50.35±0.52 at 8 hours after administration; and about 50.88±0.42 at 24 hours after administration; and about 49.84±0.47 at 48 hours after administration, based on a baseline test of about 48.63±0.55, when the formulation is administered via perineurial, subcutaneous or intramuscular administration.

Any of the embodiments set forth above, which provide an effect further characterized by a mean heat pain detection threshold of about 49.11±0.48 at 72 hours after administration.

Any of the embodiments set forth above, which provide an effect characterized by a cool detection threshold test in which the mean lowest temperature perceived as cool from a baseline of 32 C by human patients, is as follows, in degrees C.: from about 24 to about 24.5 at 24 hours after administration; from about 24.5 to about 29.5 at 48 hours after administration; from about 27.5 to about 29.8 at 72 hours after administration; from about 29 to about 30 at 96 hours after administration; and from about 29.7 to about 30.2 at 120 hours, when the formulation is administered via subcutaneous or intramuscular administration.

Any of the embodiments set forth above, which provide an effect characterized by a mechanical pain response test in which human patients characterized the pain on stimulating the injected area 5 times with von Frey hair No. 17 on a Verbal Rank Scale of 0-10 where 0=no pain and 10=pain as bad as the patient could imagine, as follows, based on a median result for patients tested: about 1 at 2 hours after administration; about 1 at 4 hours after administration; about 1 at 8 hours after administration; from about 0 to about 0.5 at 24 hours after administration; from about 0 to about 0.5 at 48 hours after administration; from about 0 to about 1 at 72 hours after administration; from about 0 to about 1 at 96 hours after administration; and about 1 at 144 hours after administration, based on a median baseline test result of about 2, when the formulation is administered via perineurial, subcutaneous or intramuscular administration.

Any of the embodiments set forth above, which provide an effect characterized by a mechanical pain response test in which human patients characterized the pain on stimulating the injected area 5 times with von Frey hair No. 17 on a Verbal Rank Scale of 0-10 where 0=no pain and 10=pain as bad as the patient could imagine, as follows, based on a median result for patients tested: from about 1 to about 3.5 at 2 hours after administration; from about 1 to about 4 at 4 hours after administration; from about 0 to about 4.5 at 8 hours after administration; from about 0 to about 3.5 at 24 hours after administration; from about 0 to about 4 at 48 hours after administration; from about 0 to about 3 at 72 hours after administration; from about 0 to about 2.5 at 96 hours after administration;, based on a median baseline test result of from about 1 to about 2.5, when the formulation is administered via perineurial, subcutaneous or intramuscular administration.

Any of the embodiments set forth above, which provide a effect characterized by a mechanical pain response test in which human patients characterized the pain on stimulating the injected area 5 times with von Frey hair No. 17 on a Verbal Rank Scale of 0-10 where 0=no pain and 10=pain as bad as the patient could imagine, as follows based on a mean result for patients tested: from about 0.9 to about 1.5 at 2 hours after administration; from about 0.7 to about 1.3 at 4 hours after administration; from about 0.5 to about 1.3 at 8 hours after administration; from about 0.2 to about 0.8 at 24 hours after administration; from about 0.2 to about 1.0 at 48 hours after administration; from about 0.3 to about 1.3 at 72 hours after administration; from about 0.4 to about 2.1 at 96 hours after administration; and from about 0.6 to about 2.0 at 144 hours after administration, based on a mean baseline test result of about 1.6 to about 2.2, when the formulation is administered via perineurial, subcutaneous or intramuscular administration.

Any of the embodiments set forth above, which provide a effect characterized by a mechanical pain response test in which human patients characterized the pain on stimulating the injected area 5 times with von Frey hair No. 17 on a Verbal Rank Scale of 0-10 where 0=no pain and 10=pain as bad as the patient could imagine, as follows based on a mean result for patients tested: from about 0.9 to about 4 at 2 hours after administration; from about 0.5 to about 5 at 4 hours after administration; from about 0.1 to about 6 at 8 hours after administration; from about 0 to about 4 at 24 hours after administration; from about 0 to about 5 at 48 hours after administration; from about 0 to about 3 at 72 hours after administration; from about 0 to about 3 at 96 hours after administration; based on a mean baseline test result of about 1.2 to about 3.3, when the formulation is administered via perineurial, subcutaneous or intramuscular administration.

Any of the embodiments set forth above, which provide a effect characterized by a mechanical pain response test in which human patients characterized the pain on stimulating the injected area 5 times with von Frey hair No. 17 on a Verbal Rank Scale of 0-10 where 0=no pain and 10=pain as bad as the patient could imagine, as follows based on a mean result for patients tested: about 1.31±0.17 at 2 hours after administration; about 1.08±0.18 at 4 hours after administration; about 0.77±0.26 at 8 hours after administration; about 0.33±0.14 at 24 hours after administration; about 0.58±0.42 at 48 hours after administration; about 0.83±0.49 at 72 hours after administration; and about 1.08±0.66 at 96 hours after administration, based on a mean baseline mechanical pain response of about 1.85±0.3, when the formulation is administered via perineurial, subcutaneous or intramuscular administration.

Any of the embodiments set forth above, which provide a effect characterized by a mechanical pain response test in which human patients characterized the pain on stimulating the injected area 5 times with von Frey hair No. 17 on a Verbal Rank Scale of 0-10 where 0=no pain and 10=pain as bad as the patient could imagine, as follows based on a mean result for patients tested: about 1.15±0.22 at 2 hours after administration; about 0.92±0.18 at 4 hours after administration; about 1.08±0.26 at 8 hours after administration; about 0.58±0.19 at 24 hours after administration; about 0.75±0.28 at 48 hours after administration; about 1±0.33 at 72 hours after administration; and about 1.42±0.71 at 96 hours after administration, based on a mean baseline mechanical pain response of about 1.85±0.27, when the formulation is administered via perineurial, subcutaneous or intramuscular administration.

Any of the embodiments set forth -above, which provide a effect characterized by a mechanical pain response test in which human patients characterized the pain on stimulating the injected area 5 times with von Frey hair No. 17 on a Verbal Rank Scale of 0-10 where 0=no pain and 10=pain as bad as the patient could imagine, as follows based on a mean result for patients tested of from about 0.2 to about 1.5 at about 2 to about 72 hours after administration, based on a mean baseline test result of about 1.6 to about 2.2, when the formulation is administered via perineurial, subcutaneous or intramuscular administration.

Any of the embodiments set forth above, which provide a effect characterized by a heat pain response test in which human patients characterized the pain on stimulating the site of injection with 45 C for 5 seconds on a Verbal Rank Scale of 0-10 where 0=no pain and 10=pain as bad as the patient could imagine, as follows, based on a median result for patients tested: from about 0 to about 1 at 2 hours after administration; about 0 at 4 hours after administration, about 0 at 8 hours after administration; about 0 at 24 hours after administration; from about 0 to about 1 at 48 hours after administration; from about 0 to about 1 at 72 hours after administration; from about 0 to about 0.5 at 96 hours after administration; and about 0 at 144 hours after administration, based on a median baseline test result of about 2, when the formulation is administered via perineurial, subcutaneous or intramuscular administration.

Any of the embodiments set forth above, which provide a effect characterized by a heat pain response test in which human patients characterized the pain on stimulating the site of injection with 45 C for 5 seconds on a Verbal Rank Scale of 0-10 where 0=no pain and 10=pain as bad as the patient could imagine, as follows, based on a median result for patients tested: from about 1 to about 5.5 at 2 hours after administration; from about 1 to about 5 at 4 hours after administration; about 0.5 to about 5.5 at 8 hours after administration; from about 0 to about 5 at 24 hours after administration; from about 0 to about 5 at 48 hours after administration; from about 0 to about 4.5 at 72 hours after administration; from about 0 to about 4 at 96 hours after administration; based on a median baseline test result of from about 1.5 to about 5, when the formulation is administered via perineurial, subcutaneous or intramuscular administration.

Any of the embodiments set forth above, which provide a effect characterized by a heat pain response test in which human patients characterized the pain on stimulating the site of injection with 45 C for 5 seconds on a Verbal Rank Scale of 0-10 where 0=no pain and 10=pain as bad as the patient could imagine, as follows, based on a mean result for patients tested: from about 0.5 to about 1.4 at 2 hours after administration; from about 0.2 to about 1.3 at 4 hours after administration; about from about 0.1 to about 1.1 at 8 hours after administration; from about 0 to about 0.8 at 24 hours after administration; from about 0.4 to about 1.5 at 48 hours after administration; from about 0.3 to about 1.2 at 72 hours after administration; from about 0.3 to about 1.8 at 96 hours after administration; and from about 0.5 to about 2.0 at 144 hours after administration, based on a mean baseline test result of from about 1.9 to about 2.9, when the formulation is administered via perineurial, subcutaneous or intramuscular administration.

Any of the embodiments set forth above, which provide a effect characterized by a heat pain response test in which human patients characterized the pain on stimulating the site of injection with 45 C for 5 seconds on a Verbal Rank Scale of 0-10 where 0=no pain and 10=pain as bad as the patient could imagine, as follows, based on a mean result for patients tested: from about 0.75 to about 6 at 2 hours after administration; from about 0.6 to about 6 at 4 hours after administration; about from about 0.4 to about 7 at 8 hours after administration; from about 0 to about 6 at 24 hours after administration; from about 0 to about 6 at 48 hours after administration; from about 0 to about 6 at 72 hours after administration; from about 0 to about 5 at 96 hours after administration, based on a mean baseline test result of from about 1.5 to about 5.5, when the formulation is administered via perineurial, subcutaneous or intramuscular administration.

Any of the embodiments set forth above, which provide a effect characterized by a heat pain response test in which human patients characterized the pain on stimulating the site of injection with 45 C for 5 seconds on a Verbal Rank Scale of 0-10 where 0=no pain and 10=pain as bad as the patient could imagine, as follows, based on a mean result for patients tested: about 0.69±0.24 at 2 hours after administration; about 0.92±0.42 at 4 hours after administration; about 0.69±0.38 at 8 hours after administration; about 0.5±0.29 at 24 hours after administration; about 0.92±0.53 at 48 hours after administration; about 0.75±0.49 at 72 hours after administration; and about 0.83±0.58 at 96 hours after administration, based on a mean baseline heat pain response of about 2.46±0.48, when the formulation is administered via perineurial, subcutaneous or intramuscular administration.

Any of the embodiments set forth above, which provide a effect characterized by a heat pain response test in which human patients characterized the pain on stimulating the site of injection with 45 C for 5 seconds on a Verbal Rank Scale of 0-10 where 0=no pain and 10=pain as bad as the patient could imagine, as follows, based on a mean result for patients tested: about 0.92±0.47 at 2 hours after administration; about 0.46±0.24 at 4 hours after administration; about 0.38±0.24 at 8 hours after administration; about 0.17±0.17 at 24 hours after administration; about 0.92±0.47 at 48 hours after administration; about 0.92±0.29 at 72 hours after administration, based on a mean baseline heat pain response of about 2.38±0.51, when the formulation is administered via perineurial, subcutaneous or intramuscular administration.

Any of the embodiments set forth above, which provide an effect characterized by a mechanical pain detection threshold test in human patients in which the median lowest number of the von Frey hair in which half of the stimulations produces a sensation of pain or unpleasantness is as follows: about 17 at 2 hours after administration; about 17 at 4 hours after administration; about 18 at 8 hours after administration; about 18 at 24 hours after administration; about 18 at 48 hours after administration; about 18 at 72 hours after administration; and about 16.5 at 96 hours after administration.

Any of the embodiments set forth above, which provide an effect characterized by a mechanical pain detection threshold test in human patients in which the median lowest number of the von Frey hair in which half of the stimulations produces a sensation of pain or unpleasantness is as follows: about 16 at 2 hours after administration; about 16 at 4 hours after administration; about 18 at 8 hours after administration; about 17.5 at 24 hours after administration; and about 17 at 48 hours after administration.

Any of the embodiments set forth above, which provide an effect characterized by a mechanical pain detection threshold test in human patients in which the median lowest number of the von Frey hair in which half of the stimulations produces a sensation of pain or unpleasantness is from about 16 to about 18 from about 2 to at least about 48 hours after administration, where the median baseline test is about 15.

Any of the embodiments set forth above, providing an effect characterized by a mechanical pain detection threshold test in human patients in which the mean lowest number of the von Frey hair in which half of the stimulations produced a sensation of pain or unpleasantness is from about 15.1 to about 18 from about 2 to at least about 96 hours after administration.

Any of the embodiments set forth above, which provide an effect characterized by a mechanical touch detection threshold test in human patients in which the median lowest force or number of a von Frey hair which produces a sensation of touch or pressure in human patients is from about 11 to about 14 from about 2 to at least about 96 hours after administration, where the median baseline test is about 9.

Any of the embodiments set forth above, which provide an effect characterized by a mechanical touch detection threshold test in human patients in which the median lowest force or number of a von Frey hair which produces a sensation of touch or pressure in human patients is from about 10 to about 13 from about 2 to at least about 48 hours after administration, where the median baseline test is about 9.

Any of the embodiments set forth above, providing an effect characterized by a mechanical touch detection threshold test in human patients in which the mean lowest force or number of a von Frey hair which produces a sensation of touch or pressure in human patients is from about 10.4 to about 15 from about 2 to at least about 96 hours after administration, based on a mean baseline test from about 8.8 to about 9.2.

Any of the embodiments set forth above, which provide an effect characterized by a mechanical touch detection threshold test in human patients in which the mean lowest force or number of a von Frey hair which produces a sensation of touch or pressure in human patients is from about 10.4 to about 13.7 from about 2 to at least about 48 hours after administration, where the mean baseline test is from about 8.8 to about 9.0.

Any of the embodiments set forth above, which provide an effect characterized by a warm detection threshold test in which the median lowest increase in temperature from 32 C perceived by human patients, is from about 43 to about 46.8 from a time of about 2 to at least about 48 hours after administration, based on a median baseline test from about 41.6 to about 42.6.

Any of the embodiments set forth above, providing an effect characterized by a warm detection threshold test in which the mean lowest increase in temperature from 32 C perceived by human patients occurs at from about 41.5 C to about 46.9 C from about 2 to at least about 96 hours after administration, where the mean baseline test is from about 41.1 to about 42.5.

Any of the embodiments set forth above, which provide a effect characterized by a mechanical pain response test in which human patients characterized the pain on stimulating the injected area 5 times with von Frey hair No. 17 on a Verbal Rank Scale of 0-10 where 0=no pain and 10=pain as bad as the patient could imagine, as follows based on a mean result for patients tested of from about 0.2 to about 1.5 at 2 to 72 hours after administration, based on a mean baseline test result of about 1.6 to about 2.2.

### EMBODIMENTS FOR INTERCOSTAL ADMINISTRATION

Embodiments set forth above providing local analgesia, local anesthesia or nerve blockade in a human, comprising a biocompatible, biodegradable carrier including a local anesthetic, said formulation providing an onset of local analgesia, local anesthesia or nerve blockade after intercostal administration in a human which, upon first administration, occurs less than about 6 hours after administration, and a duration of local analgesia which lasts until at least about 1 day after administration.

Embodiments set forth above, wherein local analgesia, local anesthesia, or nerve blockade is provided within from about 1 to about 3 hours after intercostal administration.

Embodiments set forth above, wherein the duration of local analgesia, local anesthesia, or nerve blockade is at least until about 2 days after intercostal administration.

Embodiments set forth above, wherein the duration of local analgesia, local anesthesia, or nerve blockade is at least until about 4 days after intercostal administration.

Embodiments set forth above, wherein the duration of local analgesia, local anesthesia, or nerve blockade is at least until about 10 days after intercostal administration.

Embodiments set forth above, wherein the time to maximum effect of local analgesia, local anesthesia, or nerve blockade occurs from about 6 hours to about 2 days after intercostal administration.

Embodiments set forth above, wherein the time to maximum effect of local analgesia, local anesthesia, or nerve blockade occurs at a time up to 9 days after intercostal administration.

Embodiments set forth above wherein the onset of local analgesia, local anesthesia, or nerve blockade occurs from about 3 to about 6 hours after intercostal administration.

Embodiments set forth above wherein the duration of local analgesia, local anesthesia, or nerve blockade is from about 44 hours to about 75 hours, when administered intercostally.

Embodiments set forth above wherein the duration of local analgesia, local anesthesia, or nerve blockade is from about 5 hours to about 110 hours after onset of effect.

Embodiments set forth above wherein the duration of local analgesia, local anesthesia, or nerve blockade is from about 30 hours to about 100 hours after onset of effect.

Embodiments set forth above wherein the duration of local analgesia, local anesthesia, or nerve blockade is from about 44 hours to about 75.0 hours after onset of effect.

Embodiments set forth above, which provides a effect characterized by a pin prick pain response test in which the degree of pain was assessed by administering pin pricks in an area innervated by the intercostal nerve and assessed by O, 1 or 2 wherein O means the subject did not feel any pinpricks, 1 means the subject felt 2 or 3 pinpricks as touch or pressure and 2 means the subject felt 2 or 3 pinpricks as sharp, as follows, based on a mean result for patients tested: from about 1 to about 2 at 1 hour after administration; from about 0.5 to about 1.5 at 2 hours after administration; from 0 to about 1 at 6 hours after administration; from about 0 to about 0.75 at 24 hours after administration.

Embodiments set forth above, which provide 100% sensory block based on a pin prick test from 6 hours to 24 hours after administration.

Embodiments set forth above which provide 100% sensory block based on a pin prick test at about 2 days after administration.

Embodiments set forth above which provide 100% sensory block based on a pin prick test at about 3 days after administration.

Embodiments set forth above which provide 100% sensory block based on a pin prick test at about 4 days after administration.

Embodiments set forth above, wherein the duration of effect lasts for at least until about 4 days after administration.

Embodiments set forth above wherein said formulation provides a mean duration of analgesia/anesthesia effect from about 2 days to about 4 days.

Embodiments set forth above which provide 100% sensory block based on somesthetic testing within 2 hours after administration.

Embodiments set forth above which provide a 100% sensory block based on somesthetic testing from about 2 hours to about 24 hours after administration.

Embodiments set forth above which provides a effect characterized by a numbness response test in which human patients characterized the numbness on stimulating the site of injection on a Verbal Rank Scale of 0-10 where 0=not numb and 10=totally numb, as follows, based on a mean result for patients tested: about 0 to about 4 at 2 hours after administration; about 0 to about 3 at 6 hours after administration; about 0 to about 2 at 12 hours and from 0 to about 2 at 24 hours.

Embodiments set forth above which exhibits total numbness at 2 days after administration.

Embodiments set forth above which exhibits total numbness at 4 days after administration.

Embodiments set forth above which exhibits total numbness at 6 days after administration.

Embodiments set forth above which exhibits total numbness at 8 days after administration.

Embodiments set forth above, wherein the maximum plasma levels of local anesthetic do not exceed concentrations that cause systemic toxic reactions when administered intercostally.

Embodiments set forth above, wherein the anesthetic is bupivacaine and the mean maximum plasma concentration (Cmax) of bupivacaine does not exceed 4000 ng/mL when administered intercostally.

Embodiments set forth above, wherein the mean Cmax of bupivacaine does not exceed 250 ng/mL, when administered intercostally.

Embodiments set forth above, wherein the mean Cmax of bupivacaine do not exceed about 50 ng/mL, when administered intercostally.

Embodiments set forth above, wherein the mean Cmax of bupivacaine is from about 10 to about 20 ng/mL, when administered intercostally.

Embodiments set forth above, wherein the augmenting agent is dexamethasone and the mean Cmax of dexamethasone does not exceed 300 ng/mL.

Embodiments set forth above wherein the Cmax of dexamethasone does not exceed 250 ng/mL.

Embodiments set forth above wherein the Cmax of dexamethasone does not exceed 200 ng/mL.

A formulation for providing local analgesia in a human, comprising a plurality of controlled release microspheres comprising bupivacaine free base and a biocompatible, biodegradable polymer comprising a 65:35 DL copolymer of lactic and glycolic acid having an inherent viscosity from about 0.25 to about 0.42 dL/g, a molecular weight of about 20 kDa to about 80 kDa, and free carboxylic acid end groups, said bupivacaine free base being contained in said microspheres at a drug loading of from about 60% to about 85%, by weight, said microspheres being contained in a pharmaceutically acceptable diluent for intracostal injection at a concentration sufficient to provide a concentration of bupivacaine free base from about 4.5 mg/ml to about 36.0 mg/ml and providing a unit dose of bupivacaine free base from about 45 mg to about 360 mg, said formulation providing an onset of local analgesia at the site of administration which occurs less than about 6 hours after administration, and a duration of local analgesia which lasts for at least about 1 day after administration.

Embodiments set forth above, which further comprises a dose of a second local anesthetic in immediate release form, said second local anesthetic providing said formulation with an onset of activity not more than about 5 minutes after intercostal administration of the formulation.

The formulation for providing local analgesia in a human, comprising a plurality of controlled release microspheres comprising bupivacaine free base and a biocompatible, biodegradable polymer comprising a 65:35 DL copolymer of lactic and glycolic acid having an inherent viscosity from about 0.25 to about 0.42 dL/g, a molecular weight of about 40 kDa, and free carboxylic acid end groups, said bupivacaine free base being contained in said microspheres at a drug loading of from about 60% to about 85%, by weight, said microspheres being contained in a pharmaceutically acceptable diluent for intercostal administration at a concentration sufficient to provide a concentration of bupivacaine free base from about 4.5 mg/ml to about 36.0 mg/ml and providing a unit dose of bupivacaine free base from about 45 mg to about 360 mg, said formulation providing an onset of local analgesia at the site of administration which occurs less than about 2 hours after administration, and a duration of local analgesia which lasts for at least about 1 day after administration.

Embodiments set forth above, which further comprises a dose of a second local anesthetic in immediate release form, said second local anesthetic providing said formulation with an onset of activity not more than about 5 minutes after intercostal administration of the formulation.

### EMBODIMENTS FOR SUPERFICIAL PERONEAL ADMINISTRATION

A formulation for providing local analgesia, local anesthesia or nerve blockade in a human, comprising a biocompatible, biodegradable carrier including a local anesthetic, said formulation providing an onset of local analgesia, local anesthesia or nerve blockade after administration at a single nerve in a human which, upon first administration, occurs less than about 6 hours after administration, and a duration of local analgesia which lasts until at least about 1 day after administration to a single nerve.

Embodiments set forth above, wherein said single nerve is the superficial peroneal nerve.

Embodiments set forth above, wherein the onset of local analgesia is within 30 minutes after administration.

Embodiments set forth above, wherein the duration of local analgesia after onset is about 1 day to about 7 days.

Embodiments set forth above, wherein the local analgesia is measured by a pin prick response test in which the degree of pain was assessed by administering pin pricks in an area innervated by the superficial peroneal nerve and assessed by O, 1 or 2 wherein O means the subject did not feel any pinpricks (anesthesia), 1 means the subject felt 2 or 3 pinpricks as touch or pressure or felt one as touch or pressure and 1 as sharp (analgesia) and 2 means the subject felt 2 or 3 pinpricks as sharp.

Embodiments set forth above, wherein the maximum plasma bupivacaine concentration is less than about 25 ng/mL based on administration of 27 mg bupivacaine.

Embodiments set forth above, wherein the maximum plasma bupivacaine concentration is less than about 15 ng/mL based on administration of 27 mg bupivacaine.

Embodiments set forth above, wherein the maximum plasma bupivacaine concentration is less than about 5 ng/mL based on administration of 27 mg bupivacaine.

Embodiments set forth above which provides a block of temperature perception in a human patient up to 7 days after administration.

Embodiments set forth above which provides a block of temperature perception in a human patient up to 5 days after administration.

Embodiments set forth above which provides a block of temperature perception in a human patient up to 2 days after administration.

Embodiments set forth above which provides a block of temperature perception in a human patient up to 1 day after administration.

Embodiments set forth above wherein the temperature change is measured by touching the assessment area with a cold alcohol swab and instructing the human “Tell me if you feel any change in temperature when I touch this swab to your skin” wherein a “yes” indicates the human felt a change in temperature and a “no” indicates that the human did not fell a change in temperature.

Embodiments set forth above which provides an onset of numbness in a human patient within 30 minutes after administration.

Embodiments set forth above wherein the numbness is measured by a numbness response test in which human patients characterize the numbness upon stimulation of the site of injection on a Verbal Rank Scale of 0-10 where 0=not numb and 10=totally numb.

Embodiments set forth above, which provides a effect characterized by a numbness response test in which human patients characterized the numbness on stimulating the site of injection on a Verbal Rank Scale of 0-10 where 0=not numb and 10=totally numb, as follows, based on a mean result for patients tested: about 0 to about 5 at 1 hours after administration; about 0 to about 4 at 6 hours after administration; about 0 to about 3 at 12 hours and from 0 to about 3 at 24 hours.

Embodiments set forth above providing local analgesia in a human, comprising a plurality of controlled release microspheres comprising bupivacaine free base and a biocompatible, biodegradable polymer comprising a 65:35 DL copolymer of lactic and glycolic acid having an inherent viscosity from about 0.2 to about 1.0 dL/g and a molecular weight of about 20 kDa to about 150 kDa, said bupivacaine free base being contained in said microspheres at a drug loading of from about 60% to about 85%, by weight, said microspheres being contained in a pharmaceutically acceptable diluent for administration at the superficial peroneal nerve at a concentration sufficient to provide a concentration of bupivacaine free base from about 4.5 mg/ml to about 36.0 mg/ml and providing a unit dose of bupivacaine free base from about 45 mg to about 360 mg, said formulation providing an onset of local analgesia at the site of administration which occurs less than about 6 hours after administration, and a duration of local analgesia which lasts for at least about 1 day after administration.

Embodiments set forth above, which further comprises a dose of a second local anesthetic in immediate release form, said second local anesthetic providing said formulation with an onset of activity not more than about 5 minutes after administration at the peroneal nerve.

Embodiments set forth above providing local analgesia in a human, comprising a plurality of controlled release microspheres comprising bupivacaine free base and a biocompatible, biodegradable polymer comprising a 65:35 DL copolymer of lactic and glycolic acid having an inherent viscosity from about 0.2 to about 1.0 dL/g and a molecular weight of about 20 kDa to about 150 kDa, said bupivacaine free base being contained in said microspheres at a drug loading of from about 60% to about 85%, by weight, said microspheres being contained in a pharmaceutically acceptable diluent for administration at the superficial peroneal nerve at a concentration sufficient to provide a concentration of bupivacaine free base from about 4.5 mg/ml to about 36.0 mg/ml and providing a unit dose of bupivacaine free base from about 45 mg to about 360 mg, said formulation providing an onset of local analgesia at the site of administration which occurs less than about 2 hours after administration, and a duration of local analgesia which lasts for at least about 1 day after administration.

Embodiments set forth above, which further comprises a dose of a second local anesthetic in immediate release form, said second local anesthetic providing said formulation with an onset of activity not more than about 5 minutes after superficial peroneal nerve administration of the formulation.

### EMBODIMENTS FOR SUPERFICIAL RADIAL NERVE ADMINISTRATION

Any of the foregoing embodiments formulation for providing local analgesia, local anesthesia or nerve blockade in a human, comprising a biocompatible, biodegradable carrier including a local anesthetic, said formulation providing an onset of local analgesia, local anesthesia or nerve blockade after administration to the superficial radial nerve in a human which, upon first administration, occurs less than about 6 hours after administration, and a duration of local analgesia which lasts until at least about 1 day after administration to a single nerve.

Embodiment above wherein said single nerve is the superficial radial nerve.

Embodiments set forth above wherein the onset of local analgesia is about 0.25 to about 6 hours after administration.

Embodiments set forth above wherein the duration of local analgesia after onset is about 15 to about 240 hours.

Embodiments set forth above wherein the local analgesia is measured by a pin prick response test in which the degree of pain was assessed by administering pin pricks in an area innervated by the superficial radial nerve and assessed by O, 1 or 2 wherein O means the subject did not feel any pinpricks (anesthesia), 1 means the subject felt 2 or 3 pinpricks as touch or pressure or felt one as touch or pressure and 1 as sharp (analgesia) and 2 means the subject felt 2 or 3 pinpricks as sharp.

Embodiments set forth above wherein the maximum plasma bupivacaine concentration is less than about 50 ng/mL based on administration of 56.25 mg bupivacaine.

Embodiments set forth above wherein the maximum plasma bupivacaine concentration is less than about 35 ng/mL based on administration of 56.25 mg bupivacaine.

Embodiments set forth above wherein the maximum plasma bupivacaine concentration is less than about 25 ng/mL based on administration of 56.25 mg bupivacaine.

Embodiments set forth above wherein the maximum plasma bupivacaine concentration is less than about 15 ng/mL based on administration of 56.25 mg bupivacaine.

Embodiments set forth above which provide a block of temperature perception in a human patient up to 7 days after administration.

Embodiments set forth above which provides a block of temperature perception in a human patient up to 5 days after administration.

Embodiments set forth above which provides a block of temperature perception in a human patient up to 2 days after administration.

Embodiments set forth above which provides a block of temperature perception in a human patient up to 1 day after administration.

Embodiments set forth above wherein the temperature change is measured by touching the assessment area with a cold alcohol swab and instructing the human “Tell me if you feel any change in temperature when I touch this swab to your skin” wherein a “yes” indicates the human felt a change in temperature and a “no” indicates that the human did not fell a change in temperature.

Embodiments set forth above which provide an onset of numbness in a human patient within 30 minutes after administration.

Embodiments set forth above wherein the numbness is measured by a numbness response test in which human patients characterize the numbness upon stimulation of the site of injection on a Verbal Rank Scale of 0-10 where 0=not numb and 10=totally numb.

Embodiments set forth above which provide a effect characterized by a numbness response test in which human patients characterized the numbness on stimulating the site of injection on a Verbal Rank Scale of 0-10 where 0=not numb and 10=totally numb, as follows, based on a mean result for patients tested: about 0 to about 5 at 1 hours after administration; about 0 to about 4 at 6 hours after administration; about 0 to about 3 at 12 hours and from 0 to about 3 at 24 hours.

Embodiments set forth above which provides total numbness (0) at 2 days after administration.

Embodiments set forth above which provides total numbness (0) at 5 days after administration.

Embodiments set forth above which provides total numbness (0) at 7 days after administration.

Embodiments set forth above wherein the anesthetic is bupivacaine and the formulation provides a Cmax of bupivacaine less than 250 ng/ml based on a 27 mg dose.

Embodiments set forth above wherein the formulation provides a Cmax of bupivacaine less than 200 ng/ml based on a 27 mg dose.

Embodiments set forth above wherein the formulation provides a Cmax of bupivacaine less than 150 ng/ml based on a 27 mg dose.

Embodiments set forth above wherein the formulation provides a Cmax of bupivacaine less than 100 ng/ml based on a 27 mg dose.

Embodiments set forth above wherein the maximum plasma bupivacaine concentration is less than about 50 ng/mL based on a 27 mg dose.

Embodiments set forth above providing local analgesia in a human, comprising a plurality of controlled release microspheres comprising bupivacaine free base and a biocompatible, biodegradable polymer comprising a 65:35 DL copolymer of lactic and glycolic acid having an inherent viscosity from about 0.2 to about 1.0 dL/g and a molecular weight of about 20 kDa to about 150 kDa, said bupivacaine free base being contained in said microspheres at a drug loading of from about 60% to about 85%, by weight, said microspheres being contained in a pharmaceutically acceptable diluent for administration at the superficial radial nerve at a concentration sufficient to provide a concentration of bupivacaine free base from about 4.5 mg/ml to about 36.0 mg/ml and providing a unit dose of bupivacaine free base from about 45 mg to about 360 mg, said formulation providing an onset of local analgesia at the site of administration which occurs less than about 6 hours after administration, and a duration of local analgesia which lasts for at least about 1 day after administration.

Embodiments set forth above which further comprises a dose of a second local anesthetic in immediate release form, said second local anesthetic providing said formulation with an onset of activity not more than about 5 minutes after administration at a single nerve.

Embodiments set forth above providing local analgesia in a human, comprising a plurality of controlled release microspheres comprising bupivacaine free base and a biocompatible, biodegradable polymer comprising a 65:35 DL copolymer of lactic and glycolic acid having an inherent viscosity from about 0.2 to about 1.0 dL/g and a molecular weight of about 20 kDa to about 150 kDa, said bupivacaine free base being contained in said microspheres at a drug loading of from about 60% to about 85%, by weight, said microspheres being contained in a pharmaceutically acceptable diluent for administration at the superficial radial nerve at a concentration sufficient to provide a concentration of bupivacaine free base from about 4.5 mg/ml to about 36.0 mg/ml and providing a unit dose of bupivacaine free base from about 45 mg to about 360 mg, said formulation providing an onset of local analgesia at the site of administration which occurs less than about 2 hours after administration, and a duration of local analgesia which lasts for at least about 1 day after administration.

Embodiments set forth above which further comprises a dose of a second local anesthetic in immediate release form, said second local anesthetic providing said formulation with an onset of activity not more than about 5 minutes after superficial radial nerve administration of the formulation.

### DESCRIPTION OF THE PREFERRED EMBODIMENTS

The following examples illustrate various aspects of the present invention. They are not to be construed to limit the claims in any manner whatsoever.

### EXAMPLE 1

### Manufacture of Bupivacaine/Polymer Microcapsules (IDLA)

In Example 1, microcapsules comprising polymer and bupivacaine are prepared as follows. An oil-in-water emulsion was formed from an aqueous solution containing a surfactant (process water) and an organic solvent (oil) solution containing drug and polymer. Following emulsification, the solvent was removed in an aqueous quench allowing the microcapsules to harden.

Materials

Process water (aqueous phase) was prepared as follows: 1 Kg of polyvinyl alcohol (PVA) was added to 100 L of water for injection (WFI). The WFI was mixed and heated to approximately 95° C. to dissolve the PVA. The dissolution of PVA required approximately 3 hours, following which the temperature of the solution was reduced to approximately 25° C. Finally, 7.3 L (6.5 Kg) of ethyl acetate NF (Spectrum) was stirred into the PVA solution to form the process water (aqueous phase) of the emulsion.

The polymer/drug solution (organic phase) was prepared as follows: 1.4 Kg of Medisorb 65:35DL-3A PLGA (inherent viscosity=0.25-0.42 dL/g, molecular weight approximately 40 kDa, “40K”), hydrophilic (acid end-groups) was dissolved in 37.3 L (33.4 Kg) of ethyl acetate NF under ambient conditions. Next, 3.6 Kg of bupivacaine base (Orgamol) was added to the polymer solution and mixed until dissolved. The quench solution consisted of approximately 2500L of WFI at about 18-22° C. The formula for preparation of this batch is given in Table 2 below:

TABLE 2

| | | Theoretical Percent |
|---|---|---|
| Material | Amount in batch | of Final Product |
| 65/35 DL PLGA, | 1.4 Kg | 28% |
| “40K”, acid end | | |
| groups | | |
| Bupivacaine base | 3.6 Kg | 72% |
| Ethyl acetate | 3.9 Kg | NA* |
| Polyvinyl alcohol | 1 Kg | NA* |
| (PVA) | | |
| Deionized water | 2600 L | NA* |

*Used in manufacture; the component is not present in the finished product or appears in trace quantity only.

Process

The organic phase and the aqueous phase were pumped simultaneously through a 1.375″ diameter by 6 element static mixer to form an emulsion. The organic phase was pumped at a rate of 2 Kg/minute and the aqueous phase at 4 Kg/minute, into the quench solution, which was being stirred mechanically. Both the organic and aqueous phases were filtered via in-line filters before they were presented at the static mixer. The quench solution was then stirred for 1.5 hour, after which the product was passed through 125 and 25 μm sieves. These sieves were present in a SWECO sanitary separator. The SWECO separator is designed for collection and drying of microcapsules and consists of a stack of two sieves present above a motor capable of providing vibratory motion. Following collection of the microcapsules in the SWECO separator, the microcapsules were dried by applying vacuum to the SWECO. The dried microcapsules were collected after approximately 60 hrs and the yield (25-125 μm) was 3.157 Kg.

### EXAMPLE 2

### Manufacture of Bupivacaine/Dexamethasone/Polymer Microcapsules (EDLA)

In Example 2, microcapsules comprising polymer, bupivacaine and an augmenting agent (dexamethasone) are prepared as follows. An oil-in-water emulsion was formed from an aqueous solution containing a surfactant (process water) and an organic solvent (oil) solution containing drug and polymer. Following emulsification, the solvent was removed in an aqueous quench allowing the microcapsules to harden.

Materials

Process water (aqueous phase) was prepared as follows: 1 Kg of polyvinyl alcohol (PVA) was added to 100 L of water for injection (WFI). The WFI was mixed and heated to approximately 95° C. to dissolve the PVA. The dissolution of PVA required approximately 3 hrs following which the temperature of the solution was reduced to approximately 25° C. Finally, 7.3L (6.5 Kg) of ethyl acetate NF (Spectrum) was stirred into the PVA solution to form the process water (aqueous phase) of the emulsion.

The polymer/drug solution (organic phase) was prepared as follows: 1.4 Kg of Medisorb 65:35DL-3A PLGA (inherent viscosity=0.25-0.42 dL/g, MW approximately 40 kDa, “40K”), hydrophilic (acid end-groups) was dissolved in 37.3 L (33.4 Kg) of ethyl acetate NF under ambient conditions. Next, 2.8 g dexamethasone (Upjohn) was added. Then, 3.6 Kg of bupivacaine base (Orgamol) was added to the polymer solution and mixed until dissolved. The quench solution consisted of approximately 2500 L of WFI at 18-22° C.

The formula for preparation of this batch is given in Table 3 below:

TABLE 3

| | | Theoretical Percent |
|---|---|---|
| Material | Amount in Batch | of Final Product |
| 65/35 DL PLGA, “40K”, | 1.4 Kg | 28% |
| acid end groups | | |
| Bupivacaine base | 3.6 Kg | 72% |
| Dexamethasone | 2.8 g (overage of 40%) | 0.04% |
| Ethyl acetate | 39.9 Kg | NA* |
| Polyvinyl alcohol (PVA) | 1 Kg | NA* |
| Deionized Water | 2600 L | NA* |

*Used in manufacture; the component is not present in the finished product or appears in trace quantity only.

Process

The organic phase and the aqueous phase were pumped simultaneously through a 1.375″ diameter by 6 element static mixer to form an emulsion. The organic phase was pumped at a rate of 2 Kg/minute and the aqueous phase at 4 Kg/minute, into the quench solution, which was being stirred mechanically. Both the organic and aqueous phases were filtered via in-line filters before they were presented at the static mixer. The quench solution was then stirred for 1.5 hours, after which the product was passed through 125 and 25 μm sieves. The sieves were present in a SWECO sanitary separator. The SWECO separator is designed for collection and drying of microcapsules and consists of a stack of two sieves present above a motor capable of providing vibratory motion. Following collection of the microcapsules in the SWECO separator, the microcapsules were dried by applying vacuum to the SWECO. The dried microcapsules were collected after approximately 60 hours and the yield (25-125 μm) was 3.365 Kg.

### EXAMPLE 2A

### Manufacture of 40K Microspheres with 3% Overage

In Example 2A, microcapsules comprising polymer, bupivacaine, and an augmenting agent (dexamethasone), having a 75% Bupivicane base load was prepared, using the materials and process of Example 2. The formula for the preparation of this batch is given in table 3A:

TABLE 3A

| | | Theoretical Percent |
|---|---|---|
| Material | Amount in Batch | of Final Product |
| 65/35 DL PLGA, “40K”, | 1.4 Kg | 28% |
| acid end groups | | |
| Bupivacaine base | 3.6 Kg | 75% |
| | | (3% overage) |
| Dexamethasone | 2.8 g (overage of 40%) | 0.04% |
| Ethyl acetate | 39.9 Kg | NA* |
| Polyvinyl alcohol (PVA) | 1 Kg | NA* |
| Deionized Water | 2600 L | NA* |

*Used in manufacture; the component is not present in the finished product or appears in trace quantity only.

### EXAMPLE 2B

### Manufacture of 40K Microspheres 10 K2 Scale Batches

In Example 2B, microcapsules comprising polymer, bupivacaine, and an augmenting agent (dexamethasone), having a 75% Bupivicane base load was prepared, using the materials and process of Example 2. The formula for the preparation of this batch is given in table 3B:

TABLE 3B

| | | Theoretical Percent |
|---|---|---|
| Material | Amount in Batch | of Final Product |
| 65/35 DL PLGA, “40K”, | 2.8 Kg | 28% |
| acid end groups | | |
| Bupivacaine base | 7.2 Kg | 72% |
| Dexamethasone | Either 5.6 g (40% | 0.04% |
| | overage) or 5.2 g (30% | |
| | overage) | |
| Ethyl acetate | 79.8 Kg | NA* |
| Polyvinyl alcohol (PVA) | 2 Kg | NA* |
| Deionized Water | 4200 L | NA* |

*Used in manufacture; the component is not present in the finished product or appears in trace quantity only.

### EXAMPLE 3

### Manufacture of 120K Microspheres

In order to produce a formulation using polymer of higher molecular weight, the same process used in Example 2 was used with a polymer of 120 kDa, e.g., 65/35 DL PLGA, “120K”, with acid end groups. The proportion of the relative amounts of drug and polymer were the same for the high molecular weight formulation (“120K”).

The formula for preparation of this batch is given in Table 4 below:

TABLE 4

| | | Theoretical Percent |
|---|---|---|
| Material | Amount in Batch | of Final Product |
| 65/35 DL PLGA, “120K”, | 1.4 Kg | 28% |
| acid end groups | | |
| Bupivacaine base | 3.6 Kg | 72% |
| Dexamethasone | 2.8 g (overage of 40%) | 0.04% |
| Ethyl acetate | 39.9 Kg | NA* |
| Polyvinyl alcohol (PVA) | 1 Kg | NA* |
| Deionized Water | 2600 L | NA* |

*Used in manufacture; the component is not present in the finished product or appears in trace quantity only.

### EXAMPLE 4

### Manufacture of 80K EDLA Micro Spheres

Materials

Process water (aqueous phase) was prepared as follows: A 1% stock solution of polyvinyl alcohol (PVA) was prepared by the addition of 30 g PVA (Spectrum) to 3.0L of deionized water and heated while mixing to 65-70° C. until dissolved. The PVA solution was cooled to ambient temperature and 9.5 to 3.0L. Next, 375 ml of the stock VA solution was diluted with 1125 mml of deionized water. Finally 90 ml (80.1 g) of ethyl acetate NF (Fisher) was stirred into the process water prior to forming the emulsion.

The polymer/drug solution (organic phase) was prepared as follows: 5.6 g of Medisorb 65:35DL PLGA (inherent viscosity=0.5-0.6 dl/g) was dissolved in 150 ml (133.5 g) of ethyl acetate NF under ambient conditions. Next, 0.0115 g dexamethasone (Upjohn) was added. Then, 14.4 g of bupivacaine base (Orgamol) was added to the polymer solution and sonicated until dissolved. Finally, the organic phase was filtered through a 0.22 μm PTFE filter. The quench solution consisted of 8 L of deionized water at room temperature (RT).

Process

The organic phase and the aqueous phase were pumped simultaneously through a ½″ diameter by 21 element static mixer (Cole Parmer) to form an emulsion. The organic phase was pumped at a rate of 500 ml/minute and the aqueous phase at 1000 ml/minute, into the quench solution, which was being stirred mechanically (500 rpm). The quench solution was then stirred for 1.5 hour, after which the product was passed through 125 and 25 μm sieves. The 25-125 μm portion was collected on 10 μm filter paper and dried 4 hours under vacuum followed by air drying overnight. The process yield was 14.2 g of bupivacaine/dexamethasone-loaded microspheres (EDLA).

### EXAMPLE 4A

### Manufacture of 80K EDLA Microspheres (Scaled-UP)

In order to produce a formulation using another polymer of higher molecular weight, the same process used in Example 2 was used with a polymer of 80 kDa, e.g., 65/35 DL PLGA, “80K”, with acid end groups. The proportion of the relative amounts of drug and polymer were the same for the high molecular weight formulation (“80K”).

The formula for preparation of this batch is given in Table 5 below:

TABLE 5

| | | Theoretical Percent |
|---|---|---|
| Material | Amount in Batch | of Final Product |
| 65/35 DL PLGA, “80K”, | 1.4 Kg | 28% |
| acid end groups | | |
| Bupivacaine base | 3.6 Kg | 72% |
| Dexamethasone | 2.8 g (overage of 40%) | 0.04% |
| Ethyl acetate | 39.9 Kg | NA* |
| Polyvinyl alcohol (PVA) | 1 Kg | NA* |
| Deionized Water | 2600 L | NA* |

*Used in manufacture; the component is not present in the finished product or appears in trace quantity only.

In Example 4, microcapsules comprising polymer, bupivacaine, and an augmenting agent (dexamethasone) are prepared as follows. An oil-in-water emulsion was formed from an aqueous solution containing a surfactant (process water) and an organic solvent (oil) solution containing drug and polymer. Following emulsification, the solvent was removed in an aqueous quench allowing the microspheres to harden.

### EXAMPLE 5

### Preparation of Injection Medium

An injection medium was prepared utilizing the ingredients as set forth below in Table 6. The medium is isotonic. The isotonic medium was prepared by mixing sodium carboxymethylcellulose, polysorbate 80, mannitol in sterile water. The resulting isotonic diluent was then filtered and terminally sterilized by autoclaving.

TABLE 6

| Ingredients | Composition (amount/mL) |
|---|---|
| Sodium Carboxymethylcellulose, | 0.0100 g |
| USP (CMC) | |
| Polysorbate 80, NF (Tween 80) | 0.00100 g |
| Mannitol, USP | 0.0500 g |
| Sterile Water for Injection, USP/EP (WFI) | qs to 1.0 mL |
| 0.01N Glacial Acetic Acid solution* | as needed |
| 0.01N Sodium Hydroxide solution* | as needed |
| Nitrogen, NF** | — |
| Total | 1.0 mL |

*Used to adjust the pH of the diluent to 7.2 to 7.6
**Provided inert atmosphere

A quantity sufficient of Sterile Water for Injection, USP/EP (WFI) was mixed in a sterilized vessel at 500 to 600 RPM. The temperature of the WFI was +15° C. to +30° C. The mixing rate was increased to create a vortex and Sodium Carboxymethylcellulose, USP (CMC) was sifted into the WFI. The mixing rate was then reduced to 500 to 600 RPM. This solution was mixed for 60±5 minutes. After the CMC was dissolved, the Polysorbate 80, NF (Tween 80) was added to the vessel. This solution was mixed for 10±3 minutes. After the Tween 80 had dispersed, the Mannitol, US/EP was added to the vessel. This solution was mixed for 10±3 minutes. After the Mannitol, US/EP had dissolved, the pH of the solution was measured. If the pH was above 7.2, then the pH was adjusted by adding small increments of 0.01N Glacial Acetic Acid. If the pH was below 7.6, then the pH was adjusted with small increments of 0.01N Sodium Hydroxide. The solution was mixed for 5±1 minutes at 500 to 600 RPM after each incremental addition. After the pH was adjusted, a quantity sufficient WFI was added to reach the final solution weight. The solution was mixed for 10±2 minutes. The pH of the solution was measured. If the pH was above 7.2, then the pH was adjusted by adding small increments of 0.01N Glacial Acetic Acid. If the pH was below 7.6, then the pH was adjusted with small increments of 0.01N Sodium Hydroxide. The solution was mixed fro 5±1 minutes at 500 to 600 RPM after each incremental addition.

A clarification filtration was performed on the resulting isotonic diluent with a 0.2 μm Millipore Durapore filter. Sterilized vials were aseptically filled with the filtered isotonic diluent. The vials were then sealed with sterilized seals. The sealed vials were then terminally sterilized in a Barriquand Sterilizer at 123° C.±1° C. for 42±1 minutes, D value 2.17.

### IN-VITRO RELEASE OF BUPIVACAINE FROM MICROCAPSULES OF EXAMPLES 1 AND 2

The in-vitro release of bupivacaine from the microcapsules of Examples 1 and 2 was examined. Dissolution was performed by using USP Apparatus 2 Paddle Method <711>at 100 rpm at 37° C. A 80 mg±3 mg of sample, irrespective of microcapsules formulation, was employed per vessel containing 900 mL of 10 mM Sodium Phosphate Buffer, pH 3.0. The samples (clear solution) were withdrawn at preset time intervals and analyzed for bupivacaine base by HPLC. The HPLC conditions are:

| | |
|---|---|
| Column: | Waters Nova-Pak, C₁₈, 150 × 3.9 mm |
| Temperature: | 25° C. |
| Flow Rate: | 2.0 mL/min |
| Mobile Phase: | 30:70 CH₃CN:H₂O with 50 mM C₆H₅O₇Na₃ with |
| | 0.2% TEA, pH 6.0 |
| Injection volume: | 50 μl |
| Detection: | 240 nm |

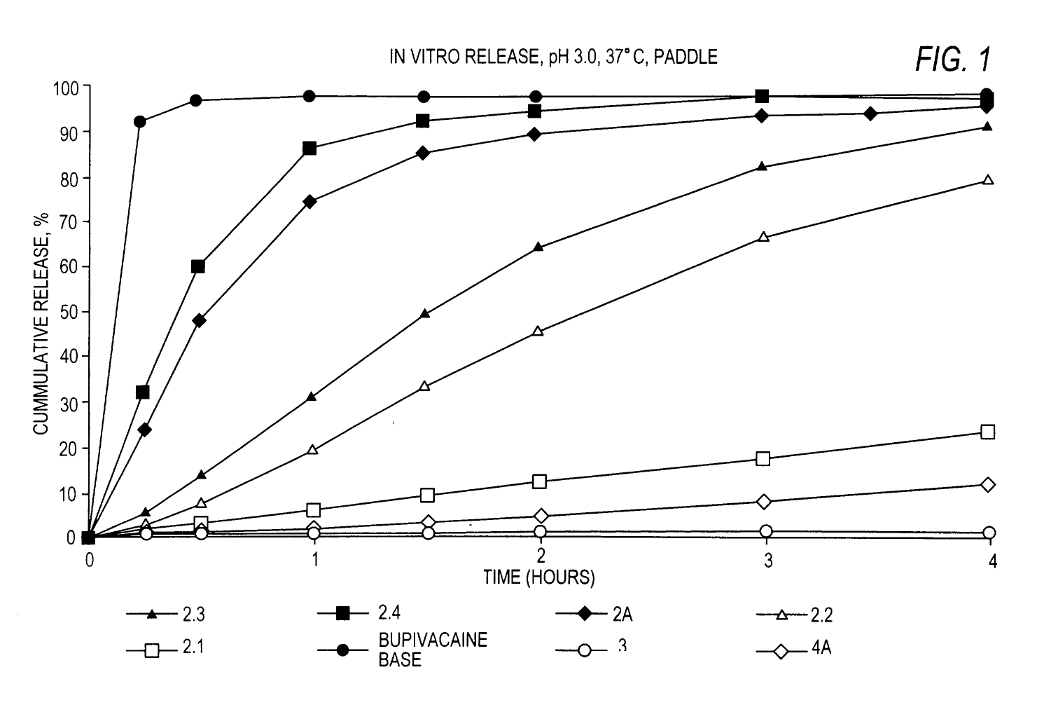

The in-vitro release of Examples 1 and 2 is shown in FIG. 1. The in-vitro release of bupivacaine from the bupivacaine-laden PLGA (approximately 40 kDa) microcapsules containing bupivacaine and dexamethasone (Example 2; alternatively referred to herein “EDLA”) is essentially identical to that of microcapsules containing no dexamethasone (Example 1; alternatively referred to herein as “IDLA”). The presence or absence of dexamethasone therefore has no impact on the release mechanism of bupivacaine from microcapsules in-vitro.

### IN-VITRO RELEASE OF BUPIVACAINE FROM 40K, 80K and 120K MICROSPHERES

The in-vitro release of bupivacaine from the microspheres of Examples 2 (batches 1-4), 2A, 3, and 4 was examined. Dissolution was performed by using USP Apparatus 2 Paddle Method at 100 rpm at 37° C. A 80 mg±3 mg of sample, irrespective of microsphere formulation, was employed per vessel containing 900 mL of 10 mM Sodium Phosphate Buffer, pH 3.0. The samples (clear solution) were withdrawn at preset time intervals and analyzed for bupivacaine base by HPLC.

The HPLC conditions are:

| | |
|---|---|
| Column: | Waters Nova-Pak, C₁₈, 150 × 3.9 mm |
| Temperature: | 25° C. |
| Flow Rate: | 2.0 mL/min |
| Mobile Phase: | 30:70 CH₃CN:H₂0 with 50 mM C₆H₅O₇Na₃ with |
| | 0.2% TEA, pH 6.0 |
| Injection volume: | 50 μl |
| Detection: | 240 nm |

Results

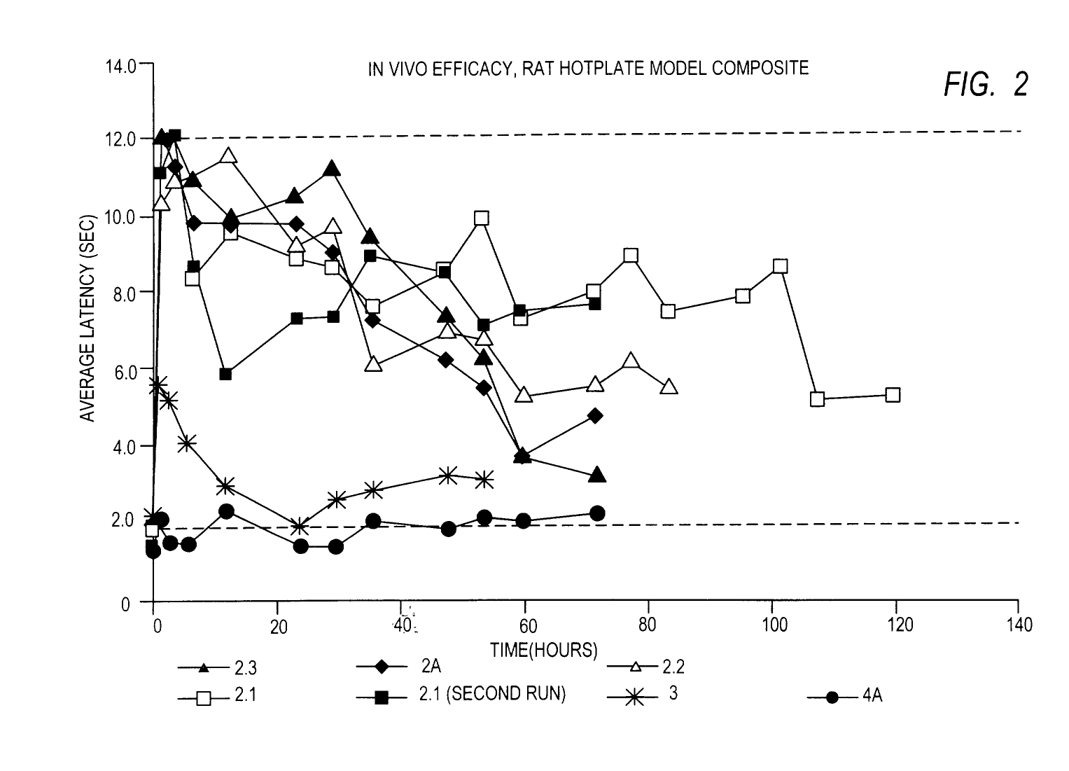

The in-vitro release of bupivacaine from the lower MW PLGA (approximately 40K) microspheres shows substantially higher release compared to the release from the higher MW (approximately 80K and approximately 120K) PLGA as shown in FIG. 2. The release from 80K and 120K microspheres was almost negligible in 4 hours. However, in 4 hours, 11.289% of drug was released from 80K polymer as compared to 1% from 120K polymer. This is to be expected based on the diffusional nature of the release where the higher MW polymer imposes a rigid barrier compared to the lower MW polymer. In addition, the hydrophilic nature of lower MW polymer assists in better hydration (wetting) of the microspheres and hence a faster dissolution rate of bupivacaine. The in-vitro release pattern of Bupivacaine from the three polymers, approximately 40K, 80K, and 120K, along with release patterns from the bupivacaine base are listed in Table 7 below:

TABLE 7
In Vitro Release of Bupivacaine (Cumulative Release % over 4 Hours)

| | Time (Hours) | | | | | | | |
|---|---|---|---|---|---|---|---|---|
| Example* | 0 | 0.25 | 0.5 | 1 | 1.5 | 2 | 3 | 4 |
| 2.1 | 0 | 2 | 3 | 6 | 9 | 12 | 17 | 23 |
| 2.2 | 0 | 3 | 8 | 19 | 33 | 45 | 66 | 79 |
| 2.3 | 0 | 6 | 14 | 31 | 49 | 64 | 82 | 91 |
| 2.4 | 0 | 32 | 60 | 86 | 92 | 94 | 97 | 97 |
| 2A | 0 | 24 | 48 | 74 | 85 | 89 | 93 | 95 |
| 3 (120K) | 0 | 1 | 1 | 1 | 1 | 1 | 1 | 1 |
| 4A (80K) | 0 | 0.6975 | 1.0075 | 1.67 | 3.1125 | 4.7838 | 7.7025 | 11.289 |
| Bupivacaine base | 0 | 92 | 96.5 | 97.09 | 96.84 | 97.035 | 97.275 | 97.665 |

*All of the Examples are based on a 5 kg scale, except for 2.4, which is based on a 10 kg scale.

The data is graphically represented in FIG. 1.

The dissolution ranges based on the above in-vitro data and the in-vivo efficacy of the formulations is listed below in Table 7A.

TABLE 7A

| TIME (Hours) | Percent Release |
|---|---|
| 0 | 0 |
| 0.25 | about 2 to about 32 |
| 0.5 | about 3 to about 60 |
| 1 | about 6 to about 86 |
| 1.5 | about 9 to about 92 |
| 2 | about 12 to about 94 |
| 3 | about 17 to about 97 |
| 4 | about 23 to about 97 |

The in-vitro release of bupivacaine from the microspheres of Examples 2B was examined. Dissolution was performed by using USP Apparatus 2 Paddle Method <711> at 100 rpm at 37° C. A 80 mg±3 mg of sample, irrespective of microsphere formulation, was employed per vessel containing 900 mL of 10 mM Sodium Phosphate Buffer, pH 3.0. The samples (clear solution) were withdrawn at preset time intervals and analyzed for bupivacaine base by HPLC. The following 24 hour dissolution release rates in Table 7B are preferred release rates which were based on batches made in accordance with the formulation of Example 2B (10 Kg scale batches).

TABLE 7B
In Vitro Release of Bupivacaine (10 Kg scale batches)
Cumulative Release % over 24 Hours

| Example | Time (Hours) | | | | | | | |
|---|---|---|---|---|---|---|---|---|
| 2B | 0 | 1.00 | 2.00 | 4.00 | 8.00 | 12.00 | 18.00 | 24.00 |

| | | | | | | | | |
|---|---|---|---|---|---|---|---|---|
| 2B.1 | 0 | 27 | 49 | 69 | 83 | 88 | 91 | 94 |
| 2B.2 | 0 | 13 | 34 | 69 | 87 | 94 | 96 | 98 |
| 2B.3 | 0.00 | 16.26 | 38.95 | 73.29 | 92.98 | 97.67 | 100.48 | 101.84 |
| 2B.4 | 0.00 | 15.47 | 36.39 | 68.13 | 90.00 | 96.40 | 99.96 | 101.64 |
| 2B.5 | 0.00 | 17.63 | 32.51 | 52.67 | 71.82 | 81.22 | 89.10 | 93.88 |
| 2B.6 | 0.00 | 30.19 | 54.12 | 74.48 | 87.61 | 92.88 | 96.81 | 99.06 |
| 2B.7 | 0.00 | 30.67 | 56.84 | 79.26 | 92.32 | 96.27 | 99.18 | 100.80 |
| 2B.8 | 0.00 | 36.13 | 58.89 | 77.48 | 88.90 | 92.67 | 96.02 | 97.93 |
| 2B.9 | 0.00 | 25.79 | 46.47 | 67.76 | 82.92 | 88.62 | 93.27 | 96.09 |
| 2B.10 | 0.00 | 25.94 | 49.39 | 73.12 | 88.70 | 94.20 | 97.94 | 99.96 |
| 2B.11 | 0.00 | 29.85 | 52.29 | 72.34 | 86.05 | 91.29 | 95.32 | 97.78 |
| 2B.12 | 0.00 | 36.21 | 65.36 | 86.92 | 95.23 | 97.80 | 100.08 | 101.46 |
| AVER- | 0.00 | 23.57 | 45.24 | 70.12 | 86.28 | 91.97 | 95.76 | 98.14 |
| AGE* | | | | | | | | |
| Std. Dev. | 0.00 | 8.17 | 10.13 | 7.77 | 6.48 | 5.23 | 3.97 | 3.03 |

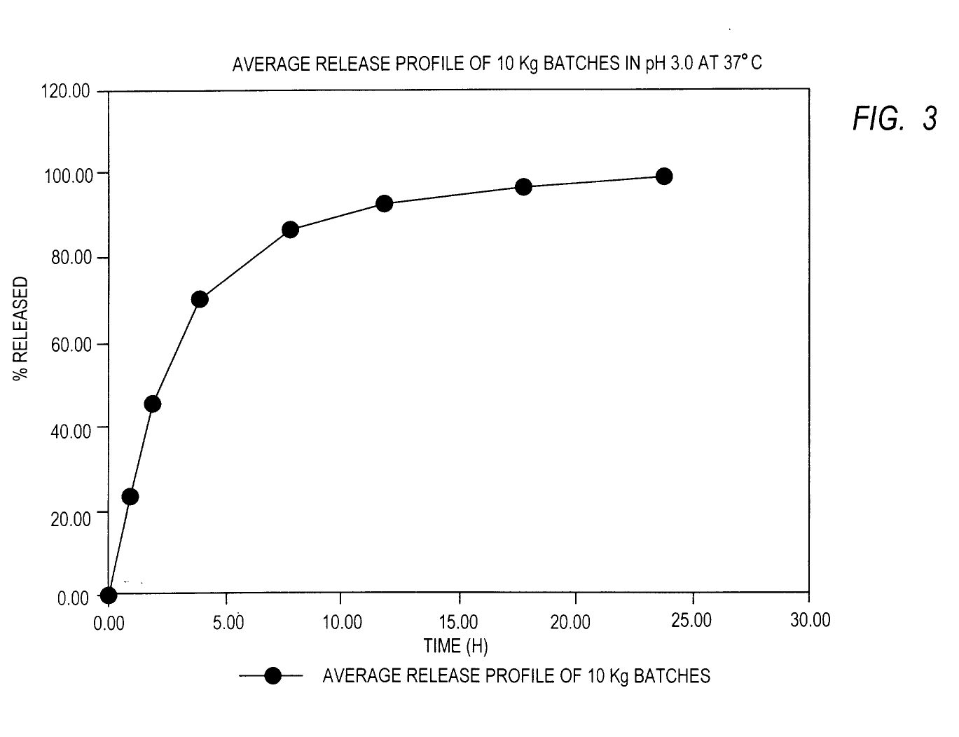

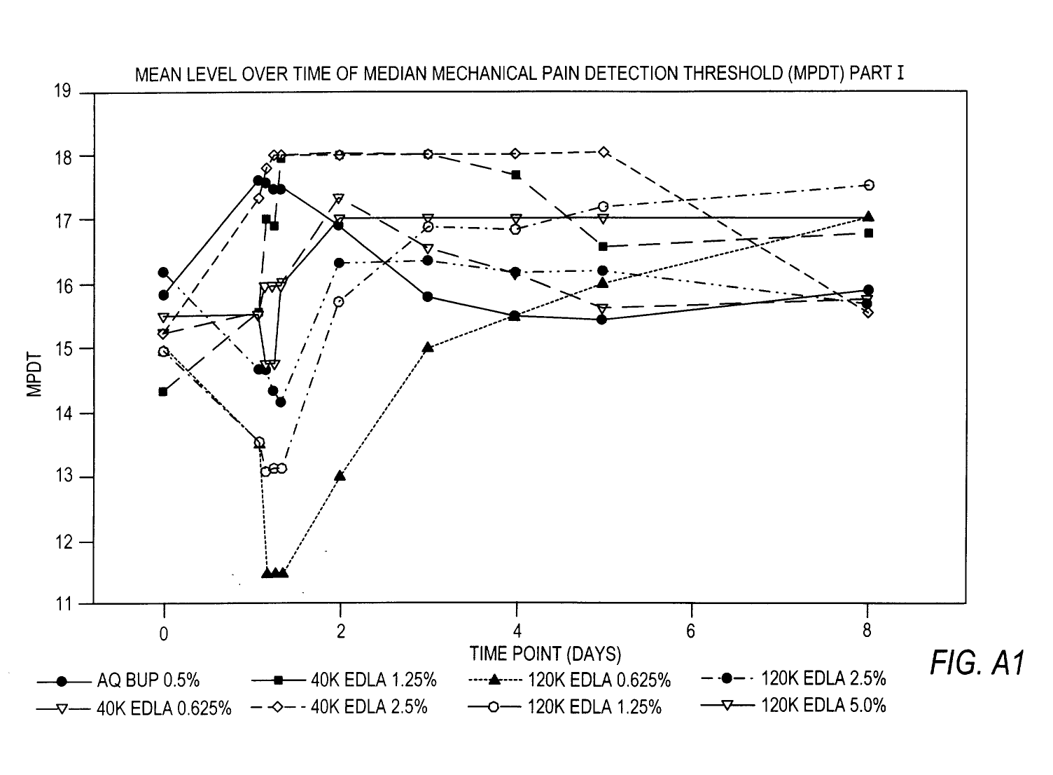

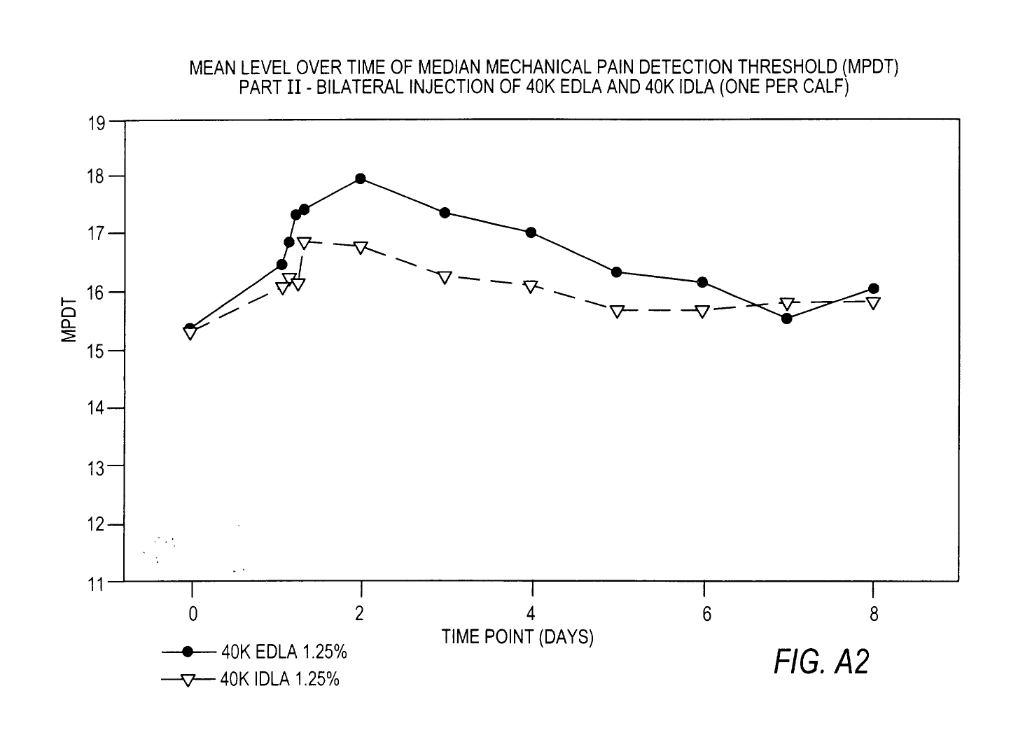

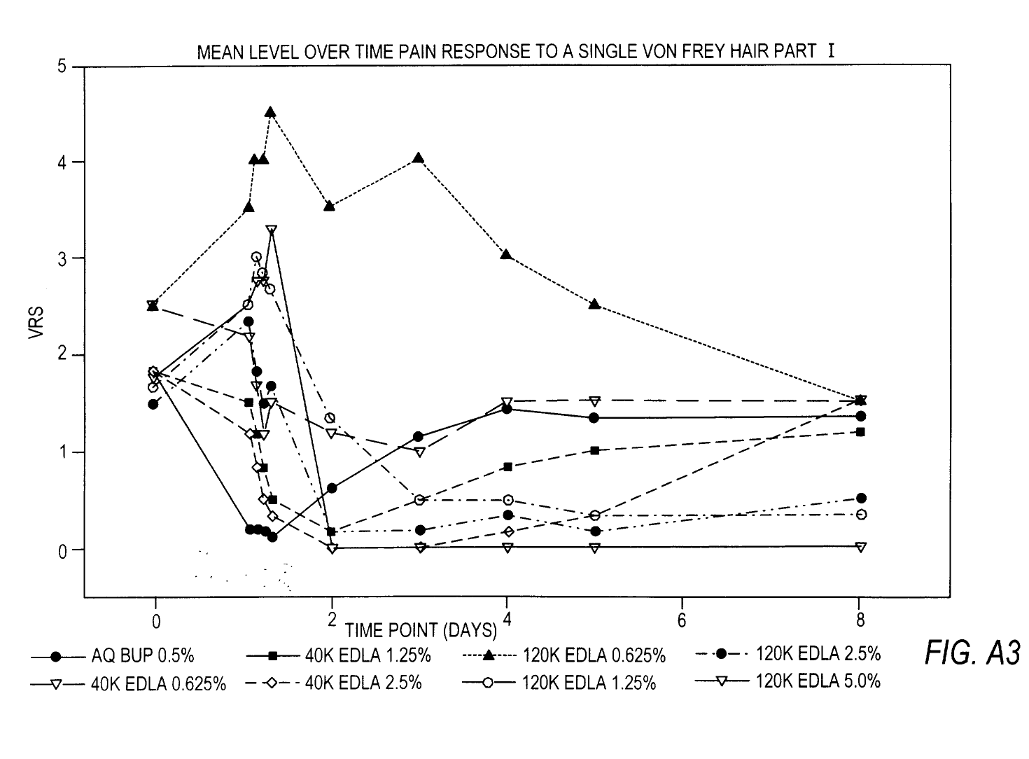

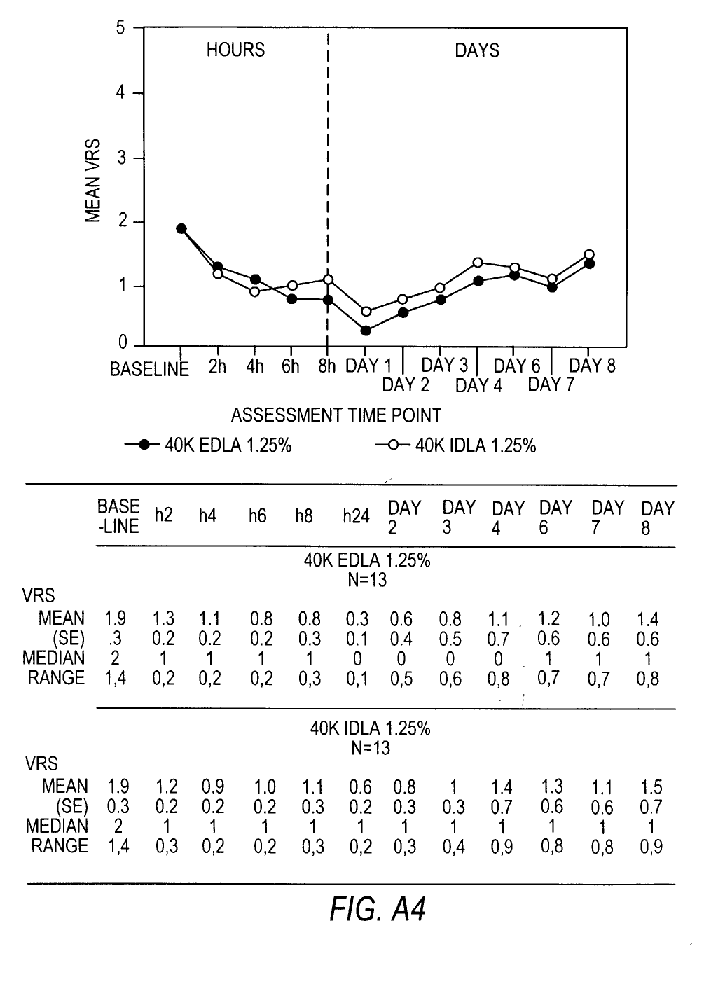

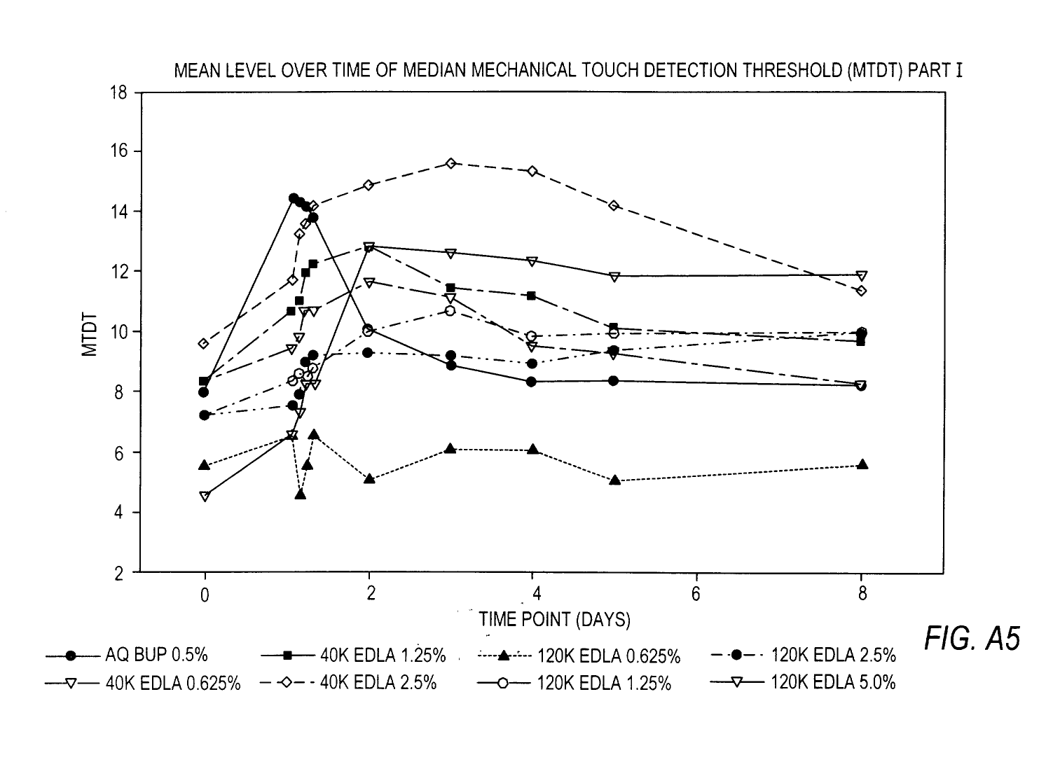

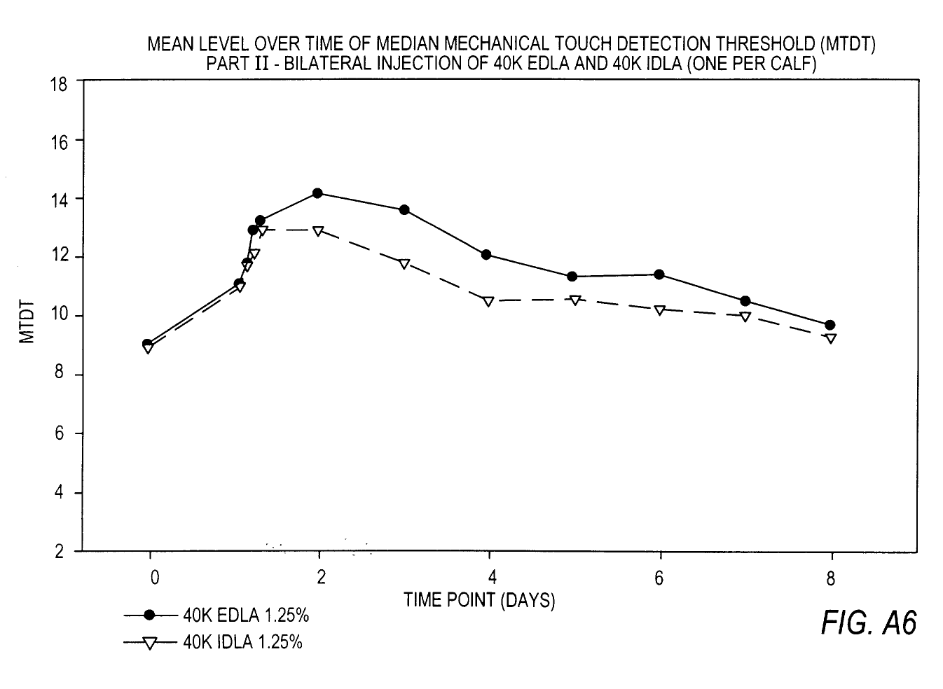

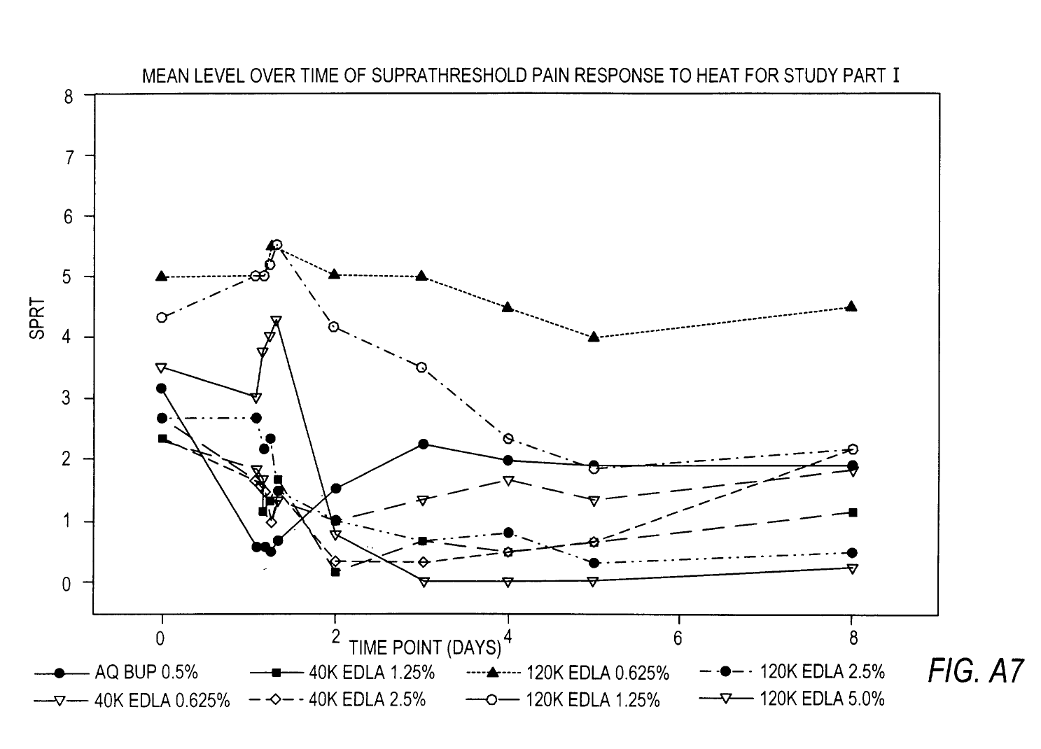

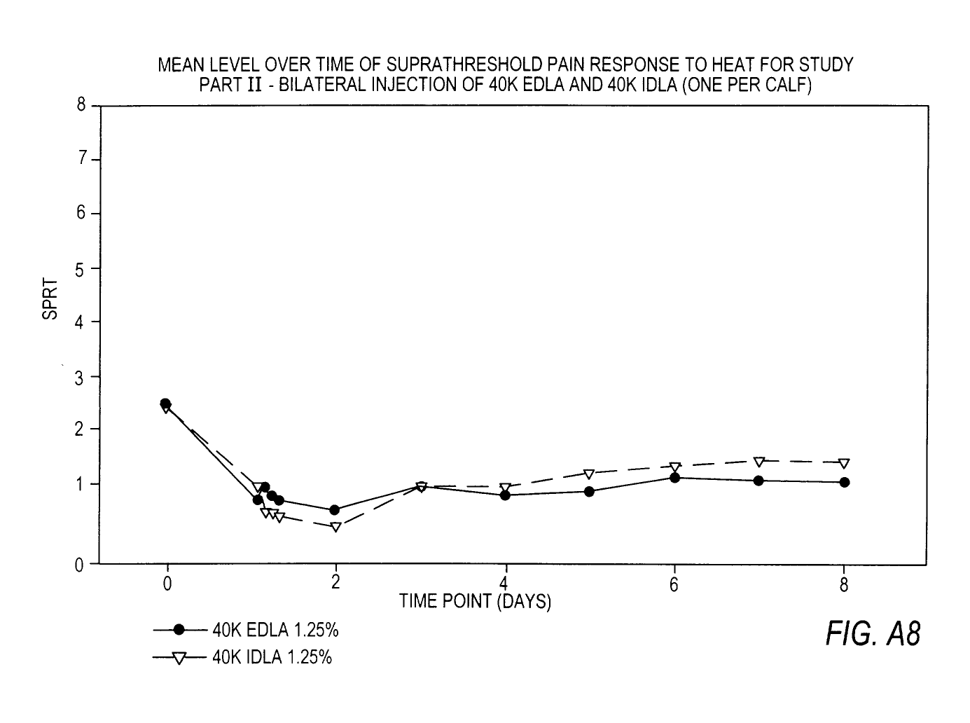

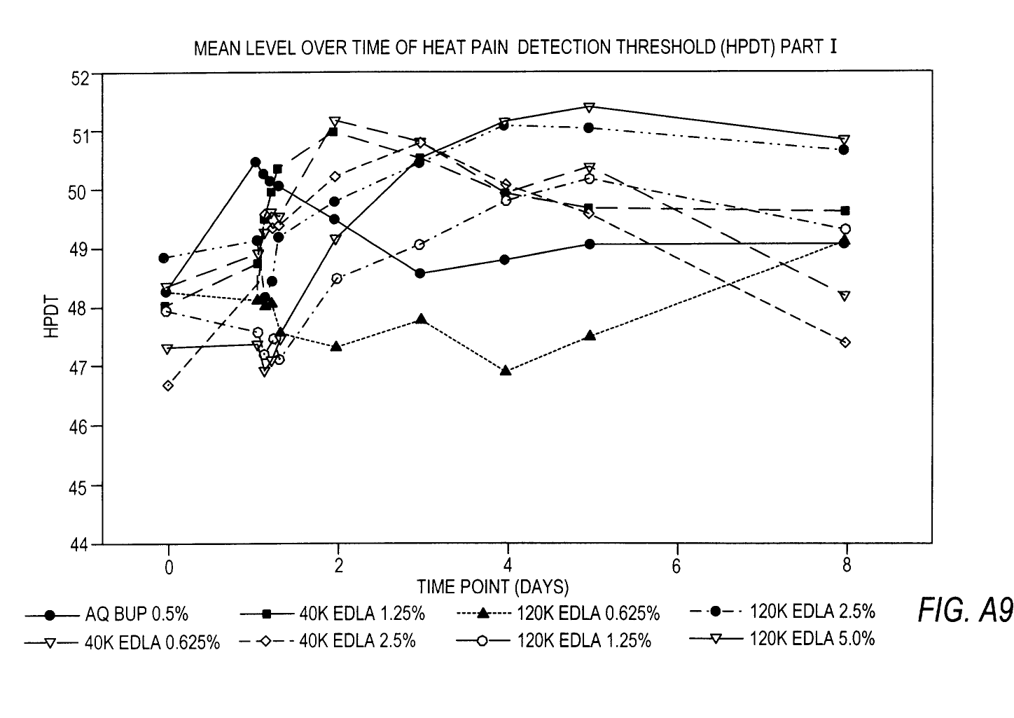

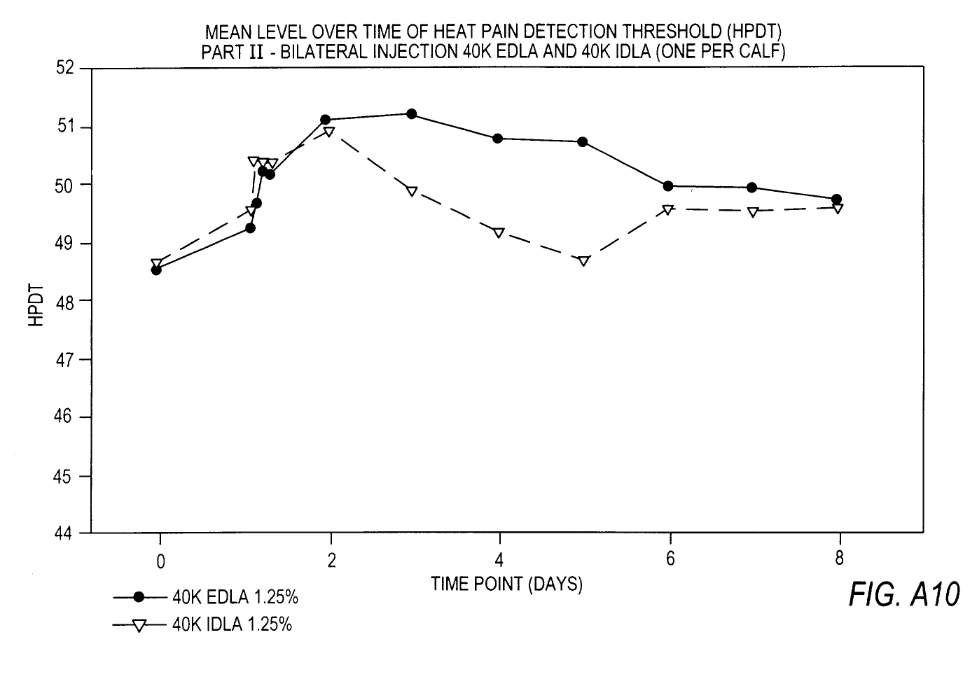

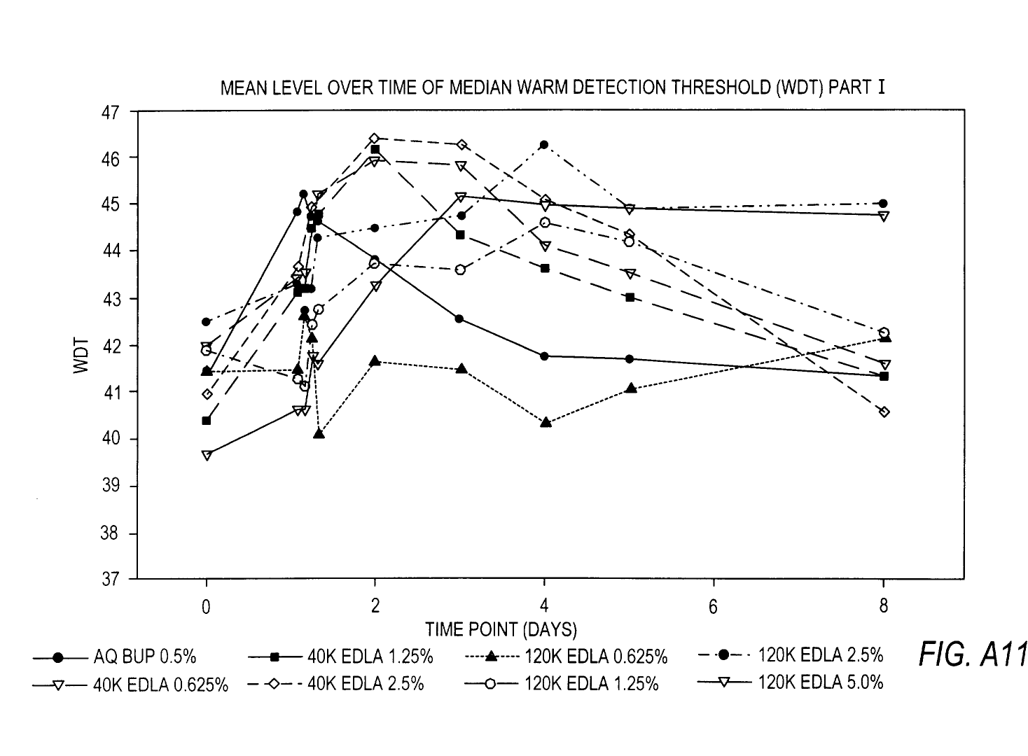

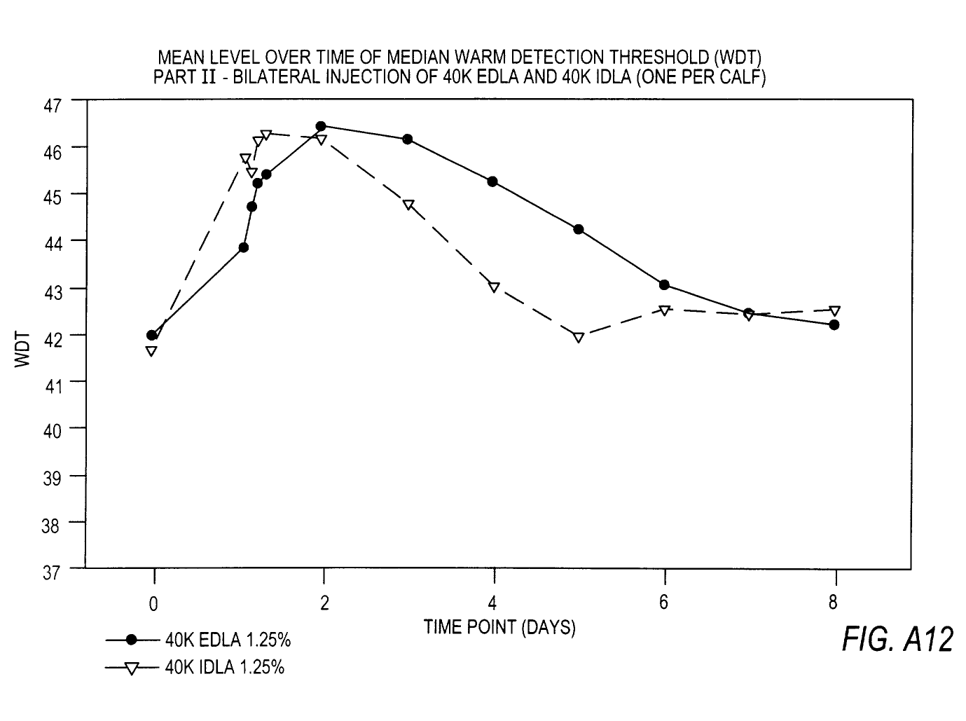

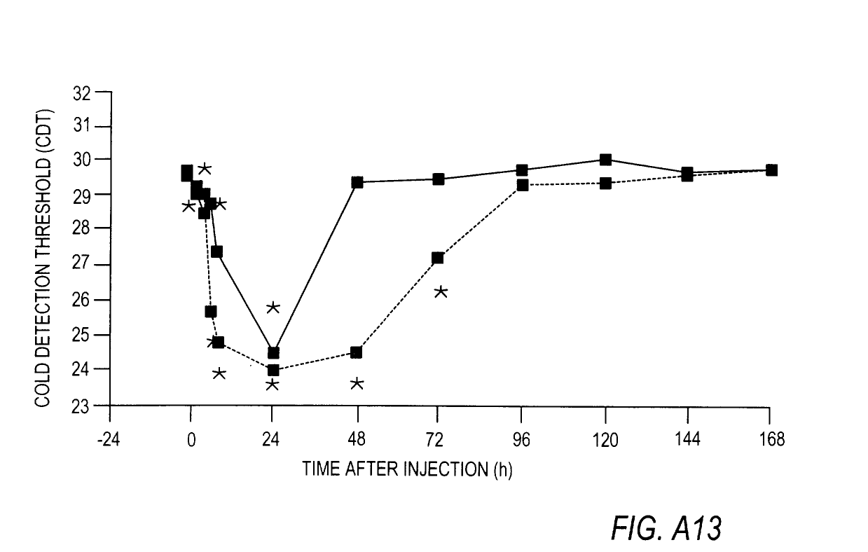

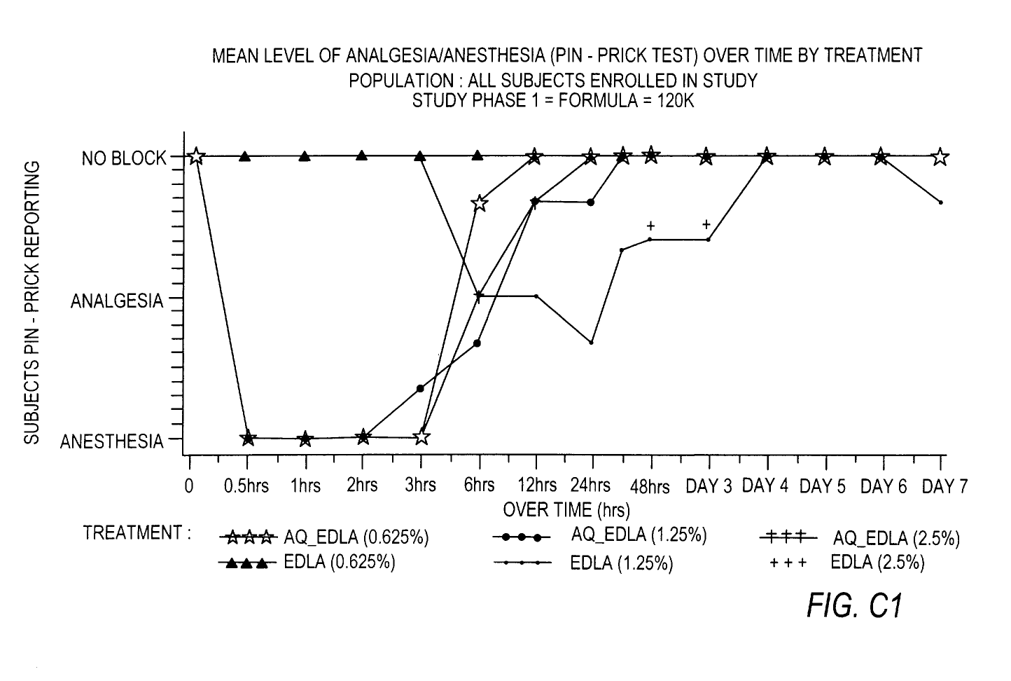

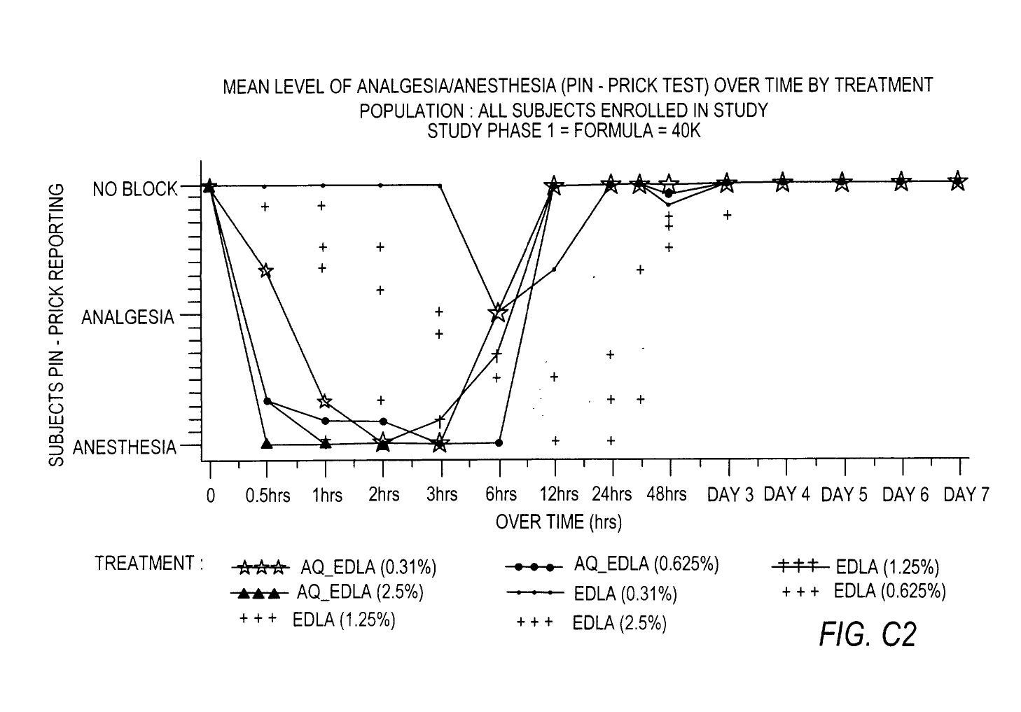

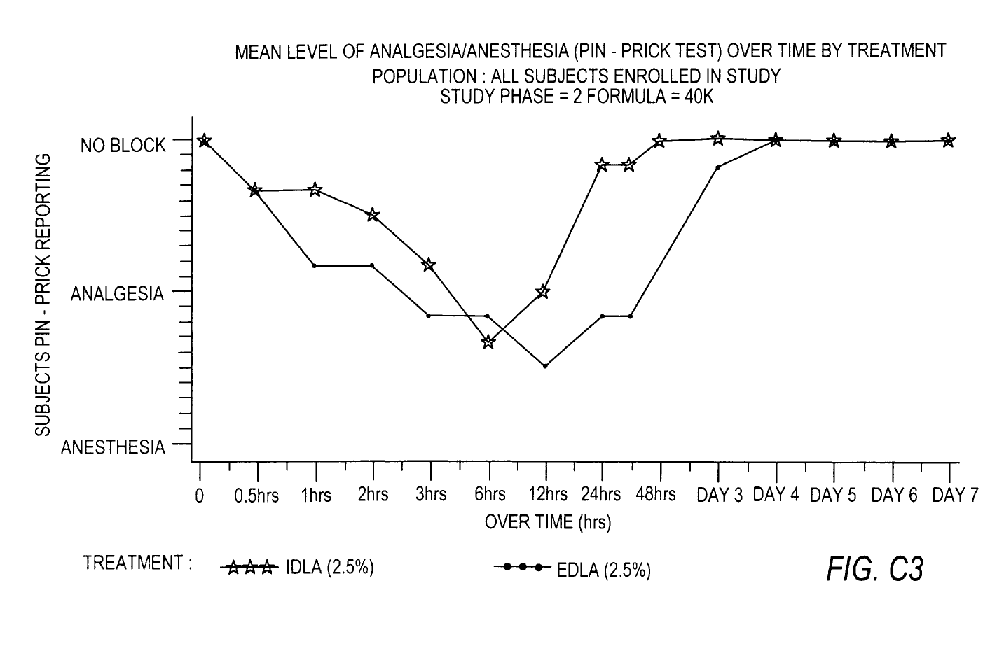

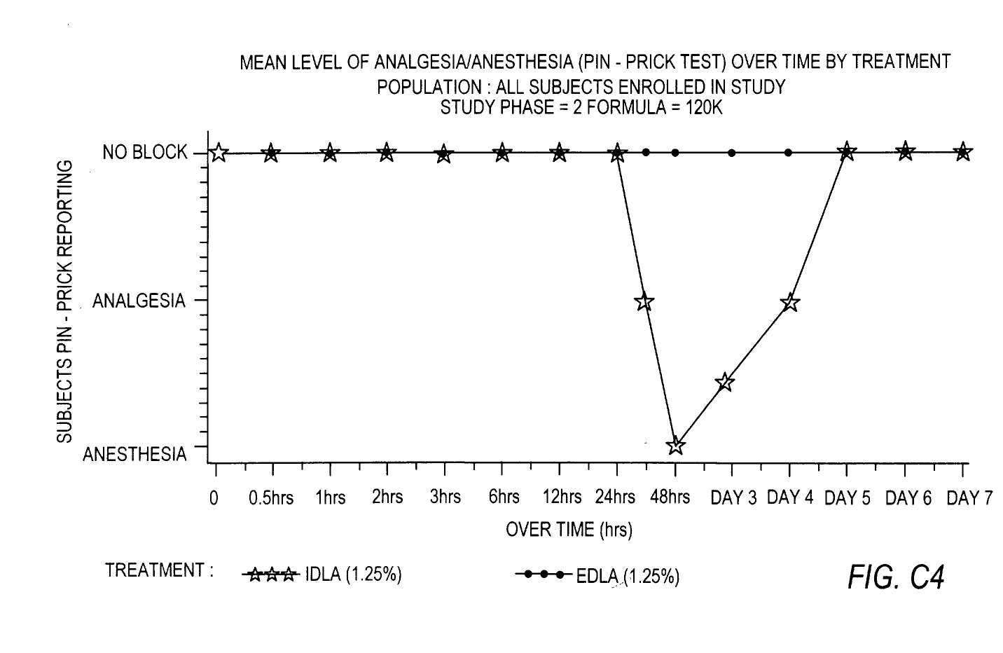

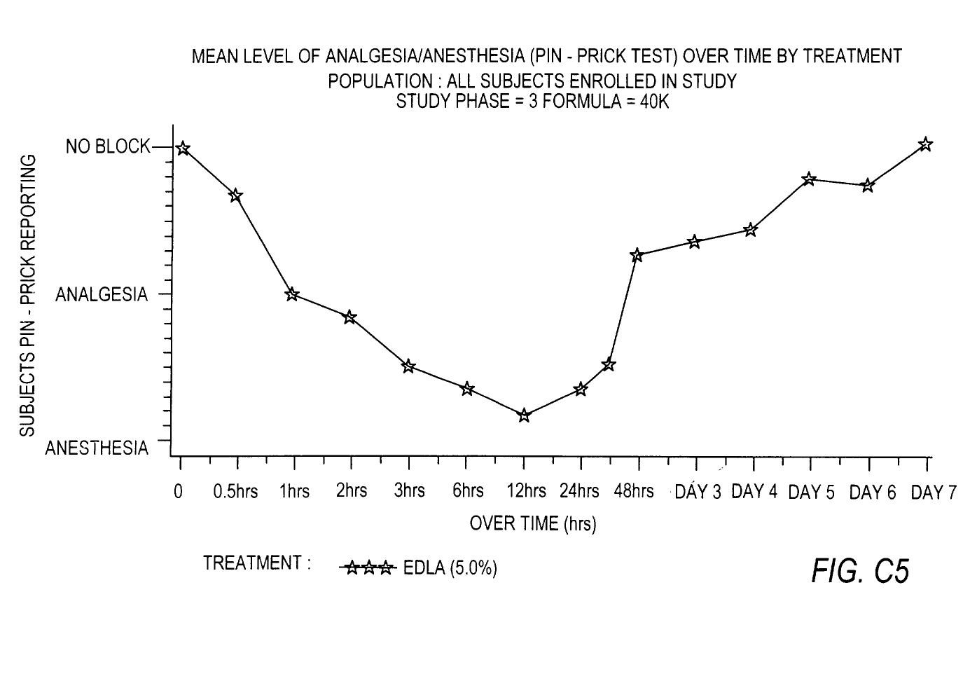

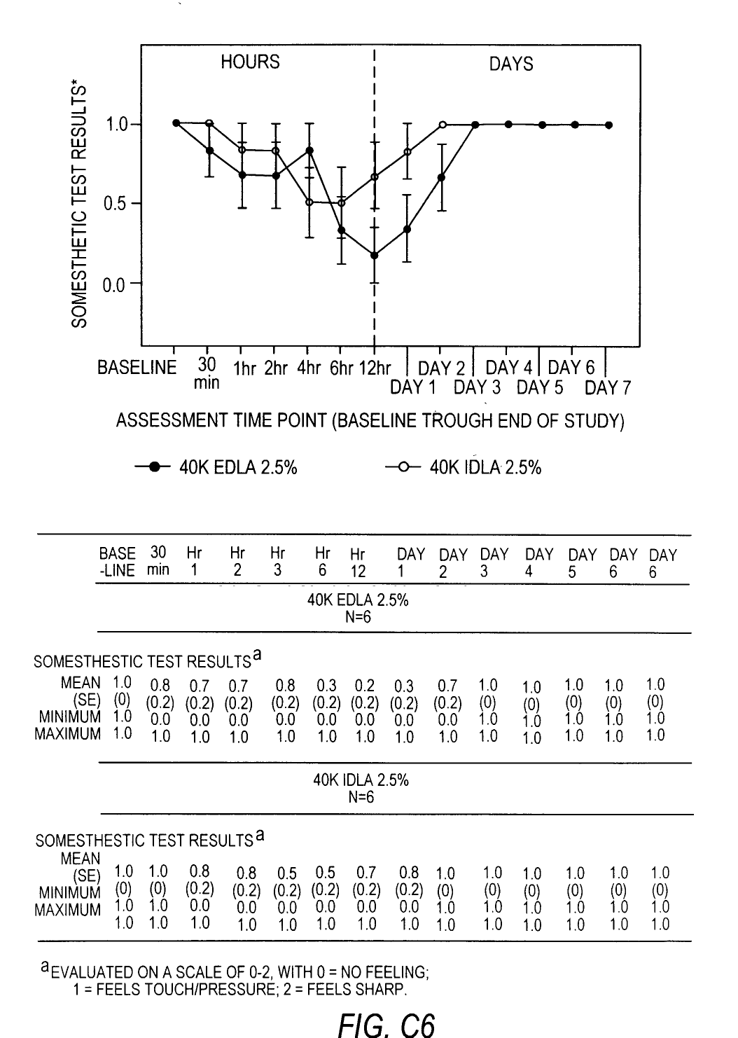

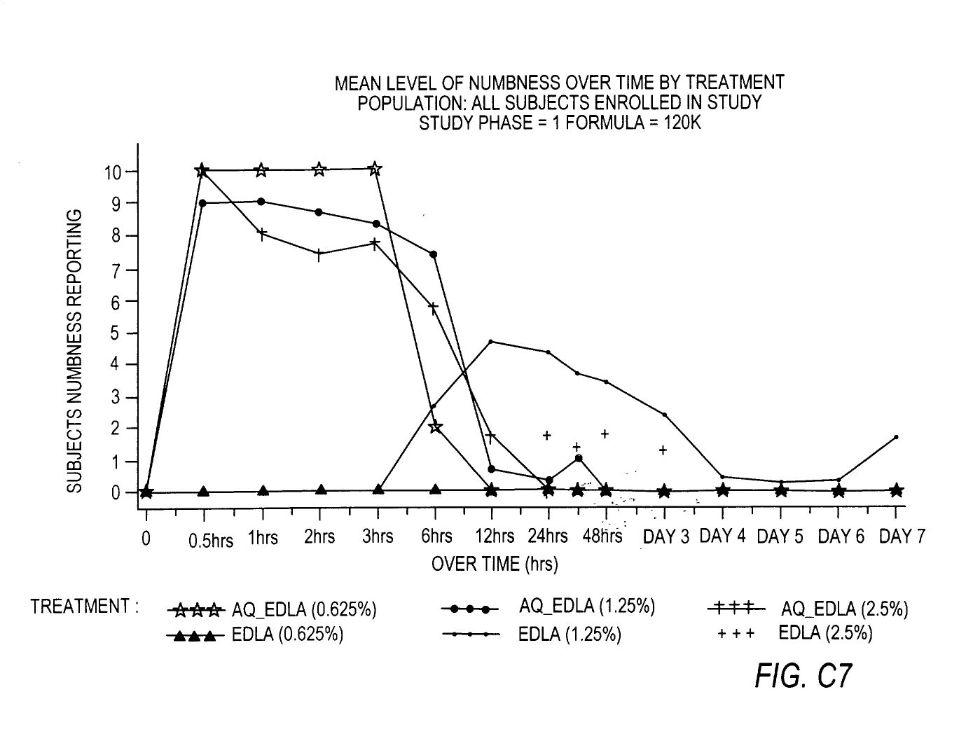

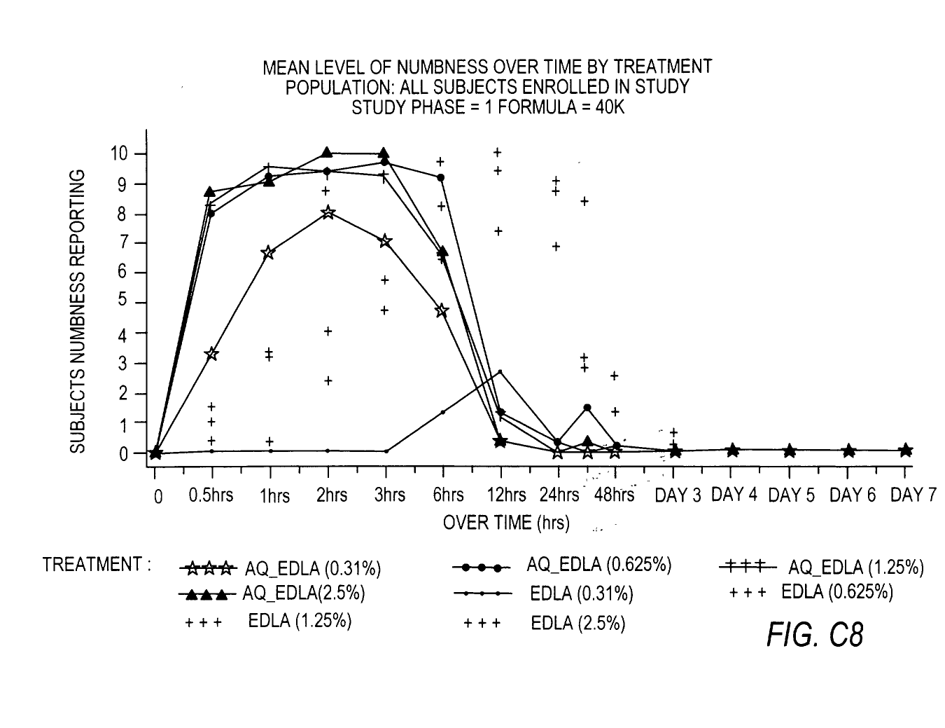

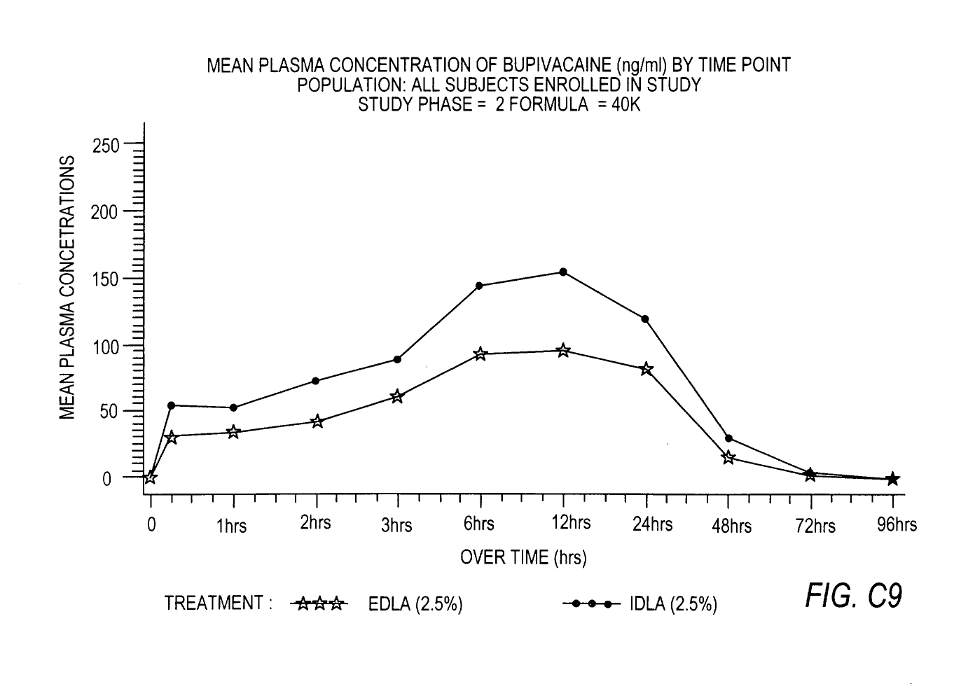

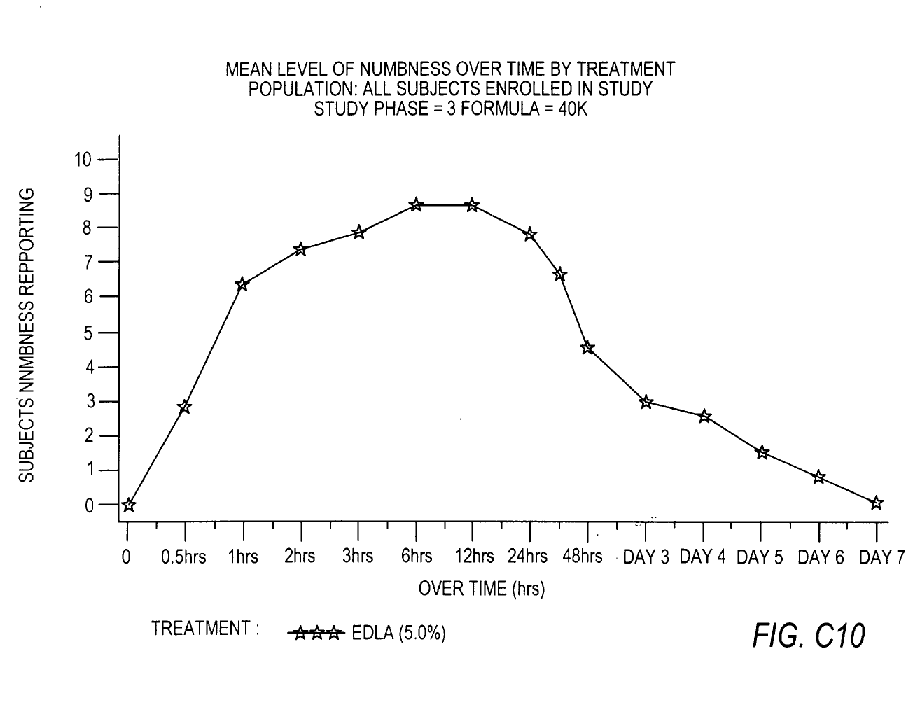

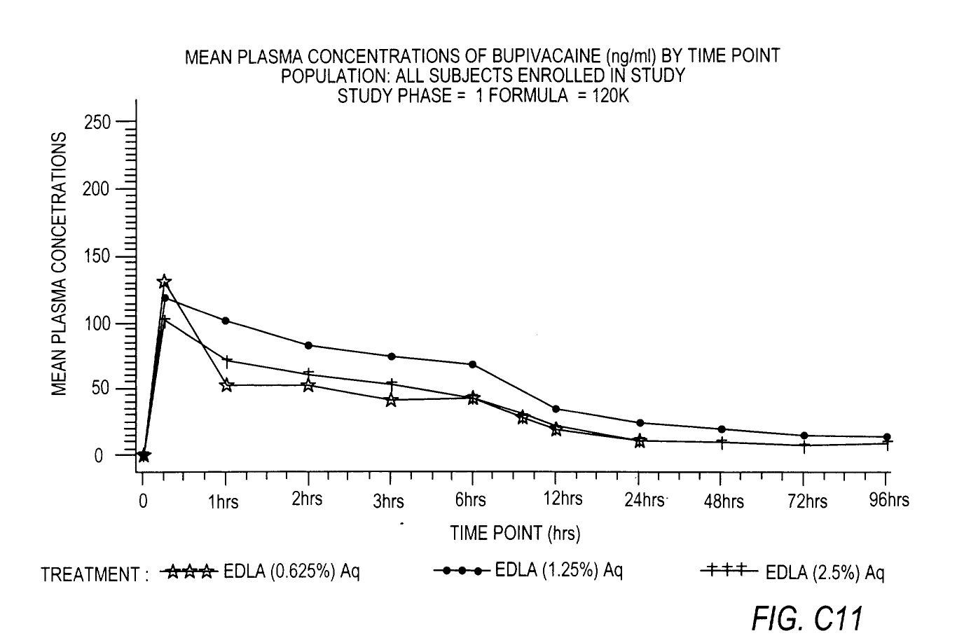

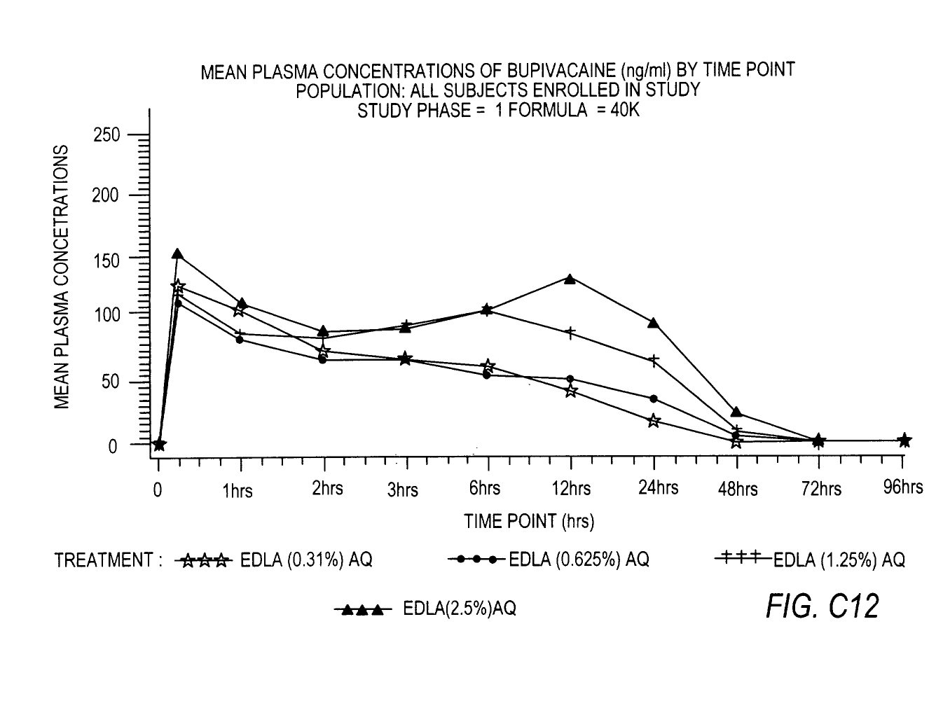

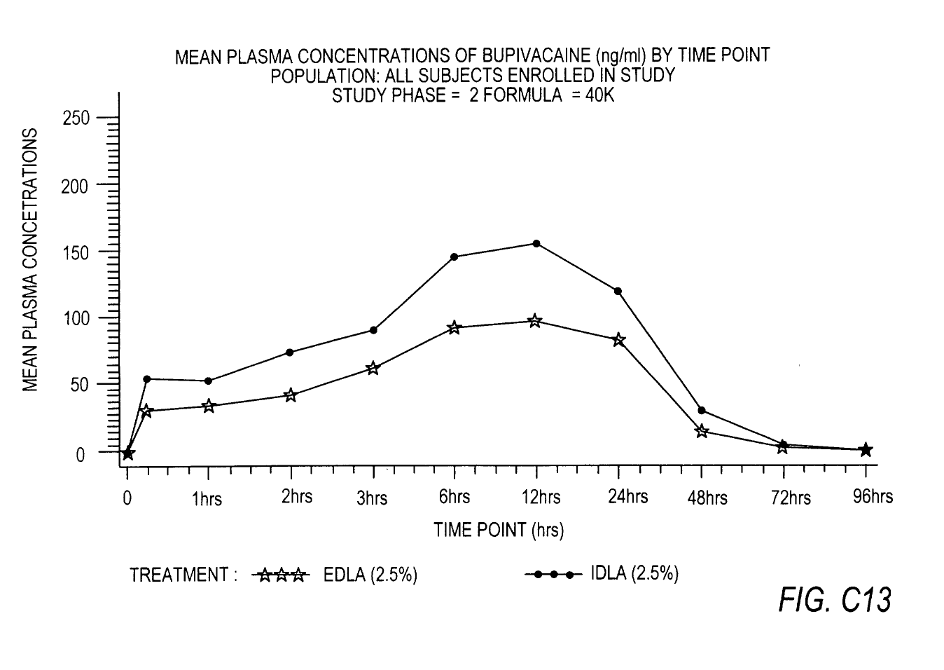

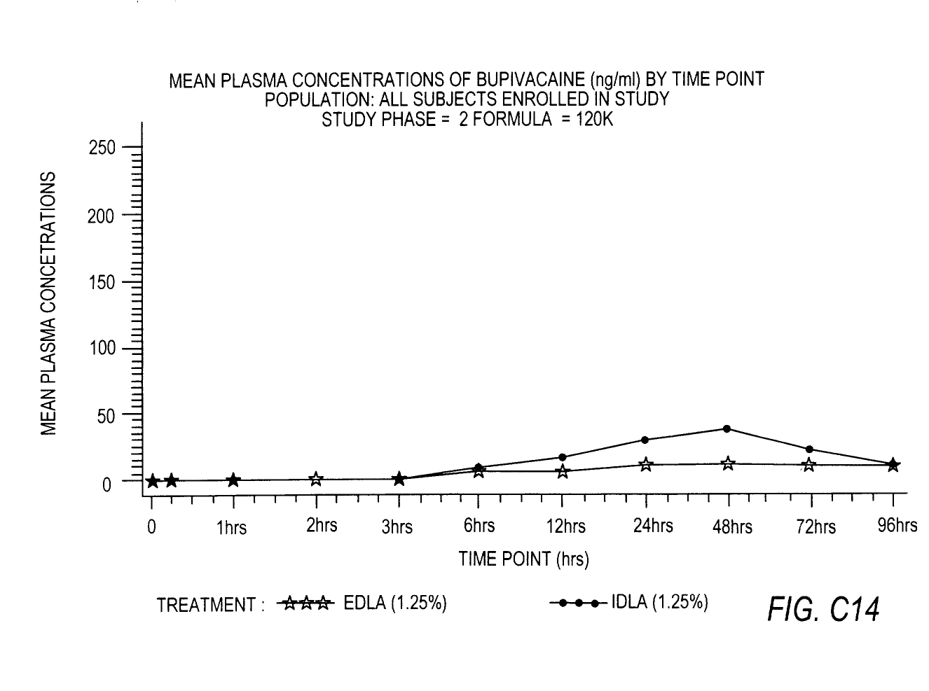

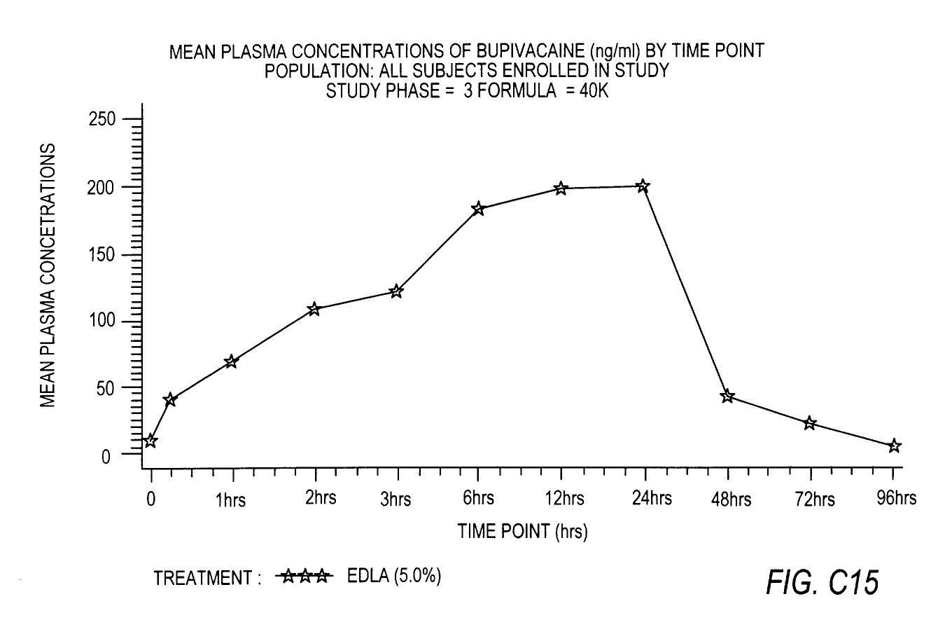

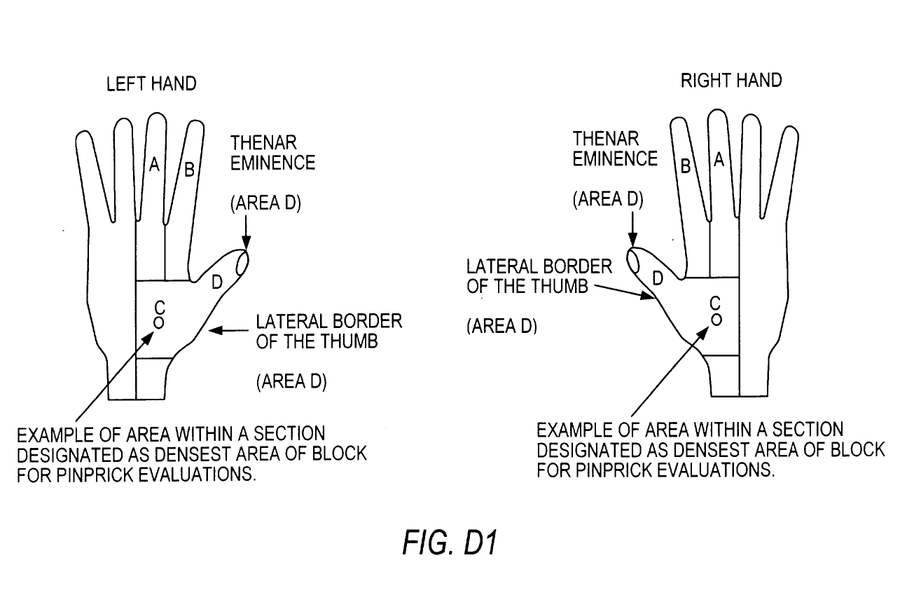

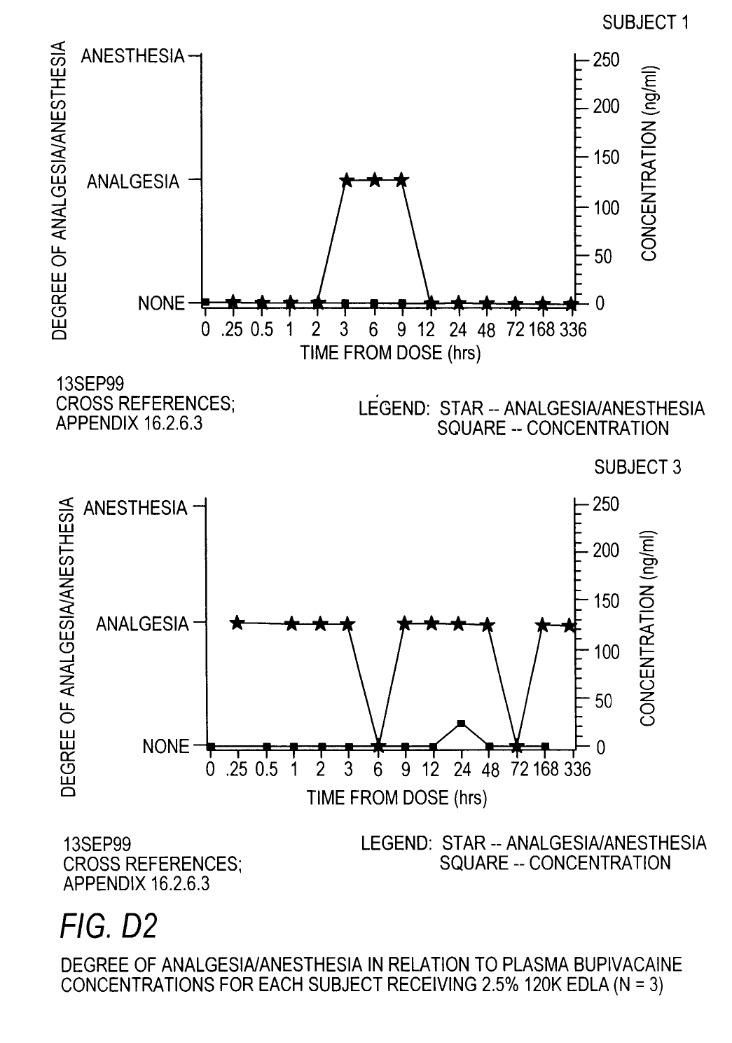

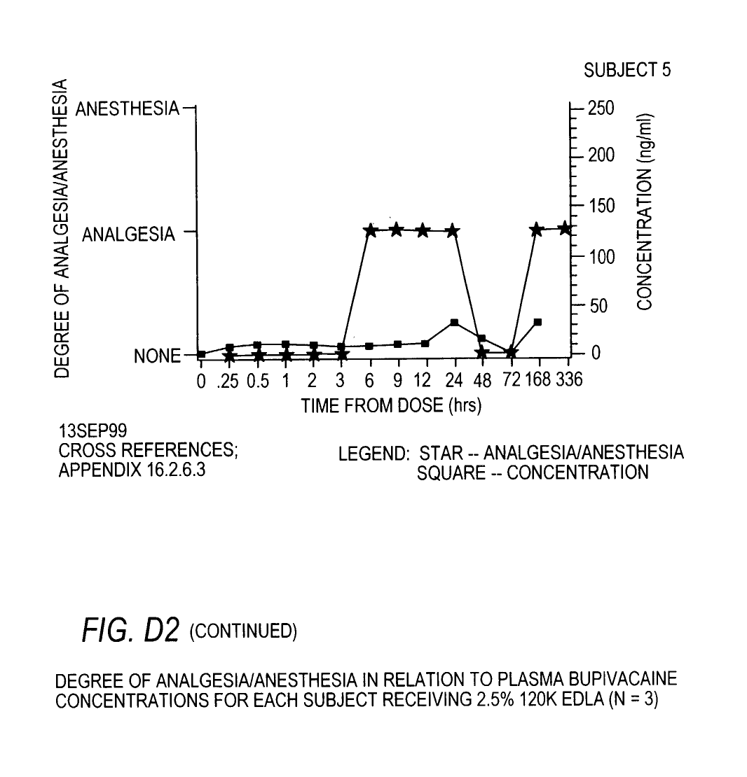

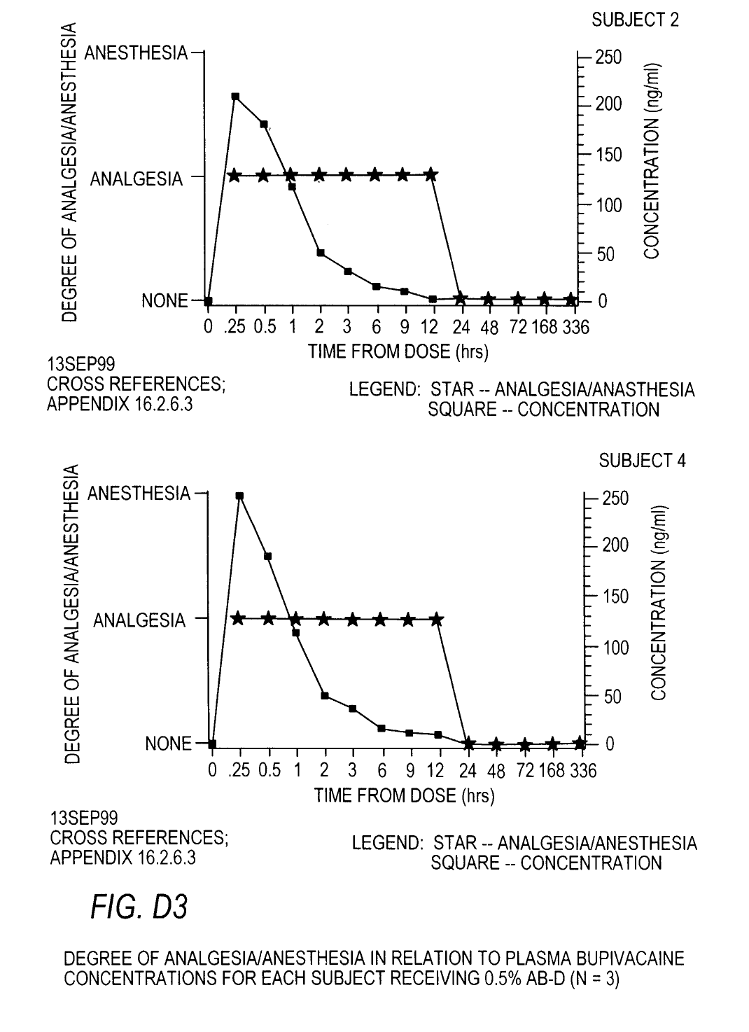

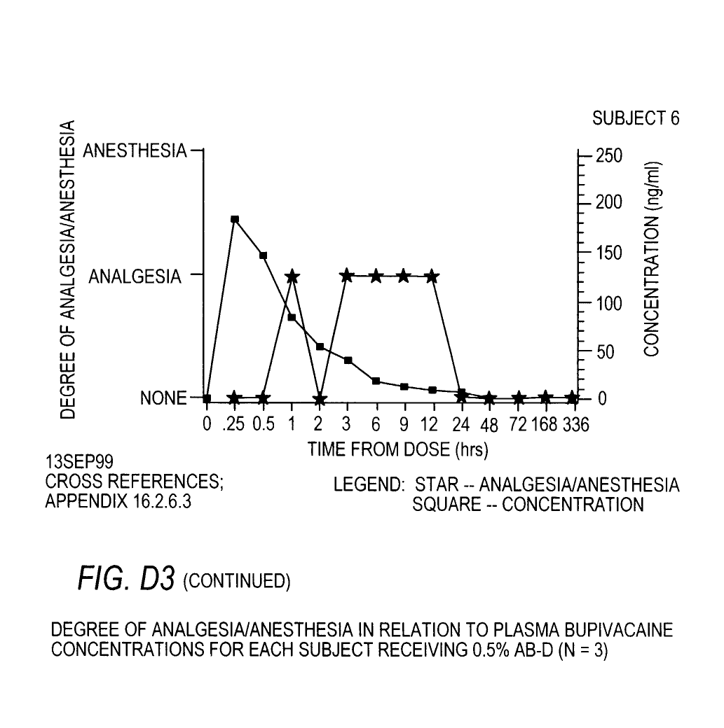

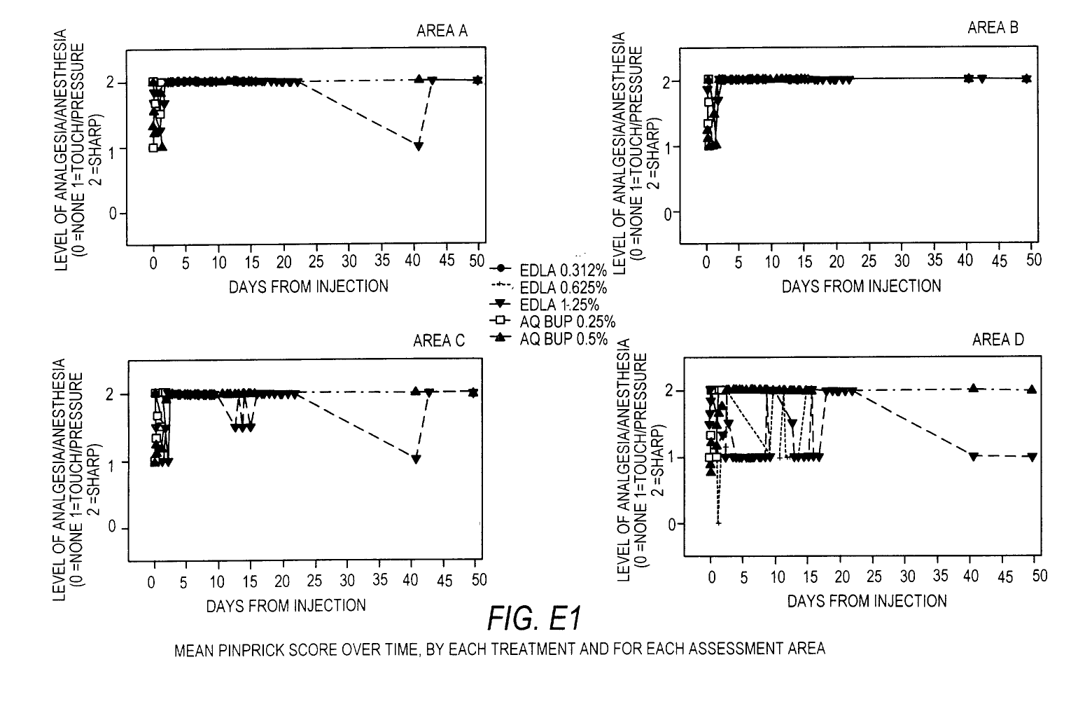

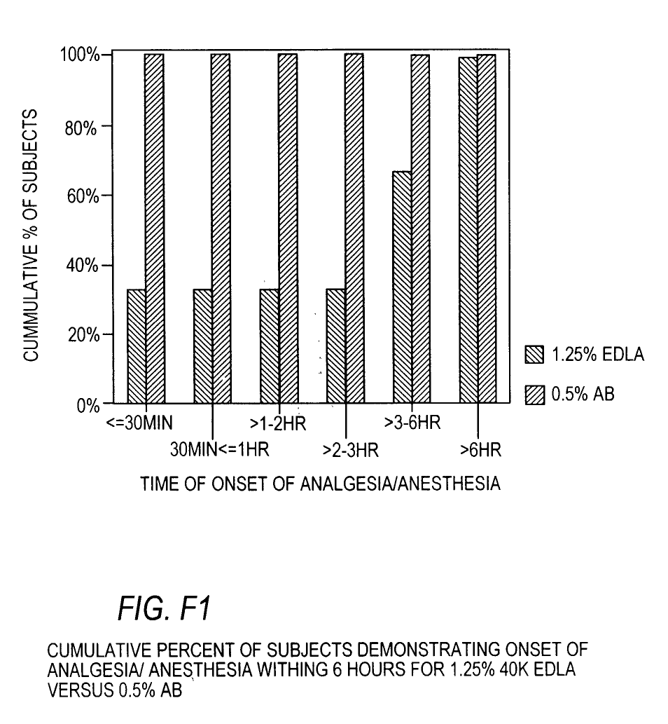

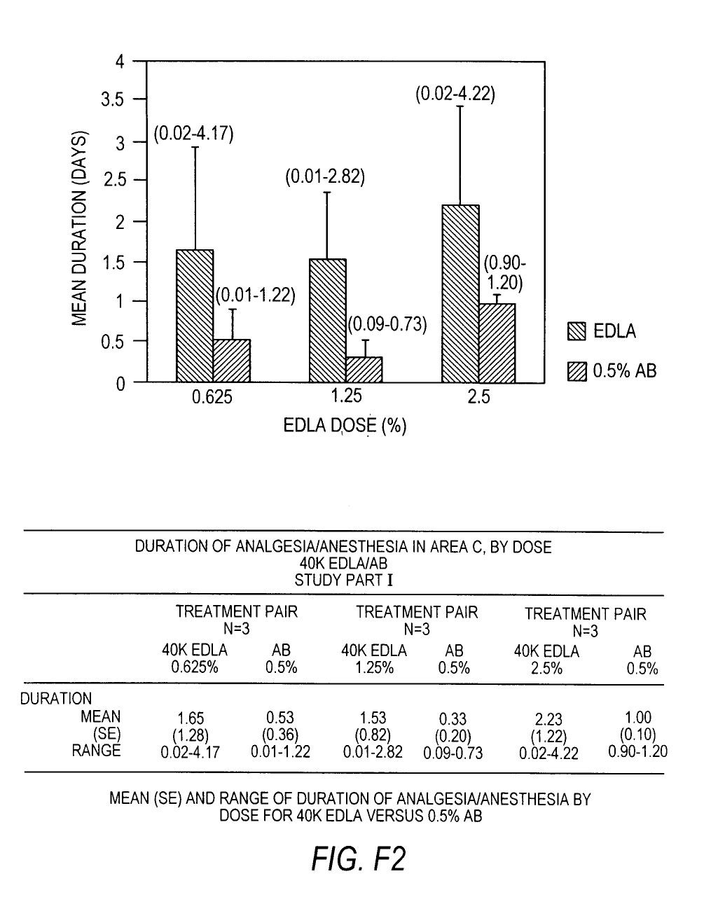

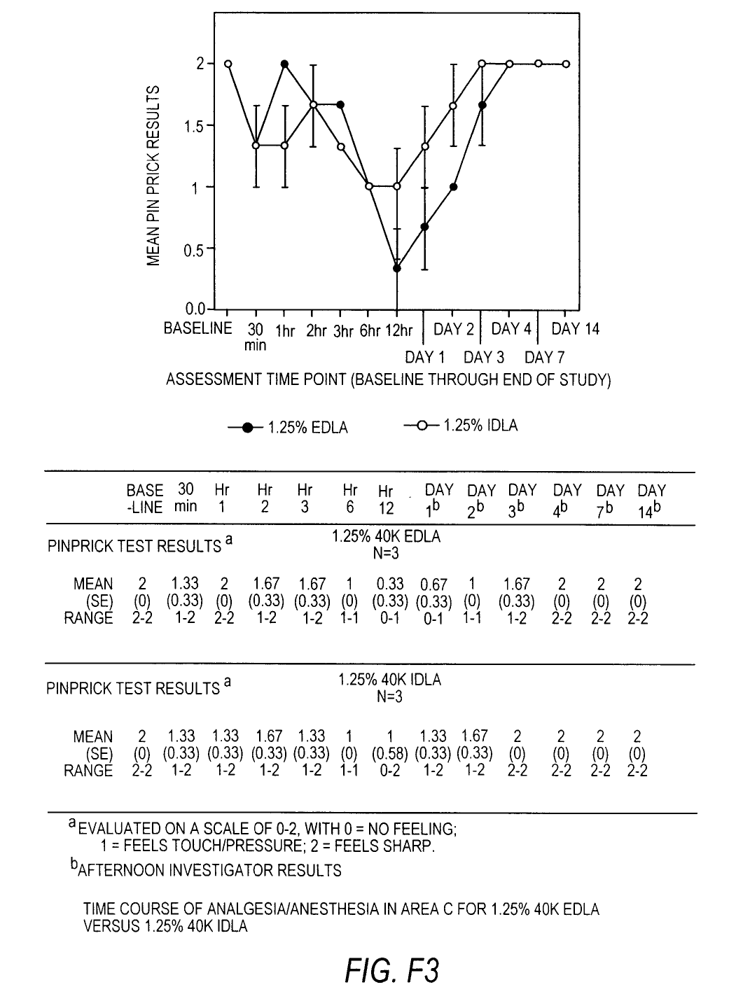

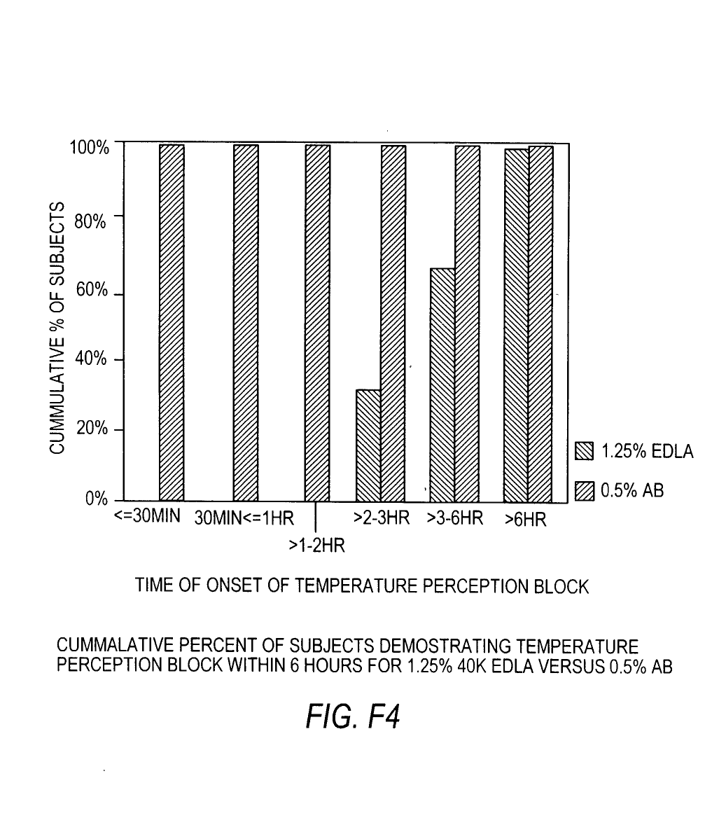

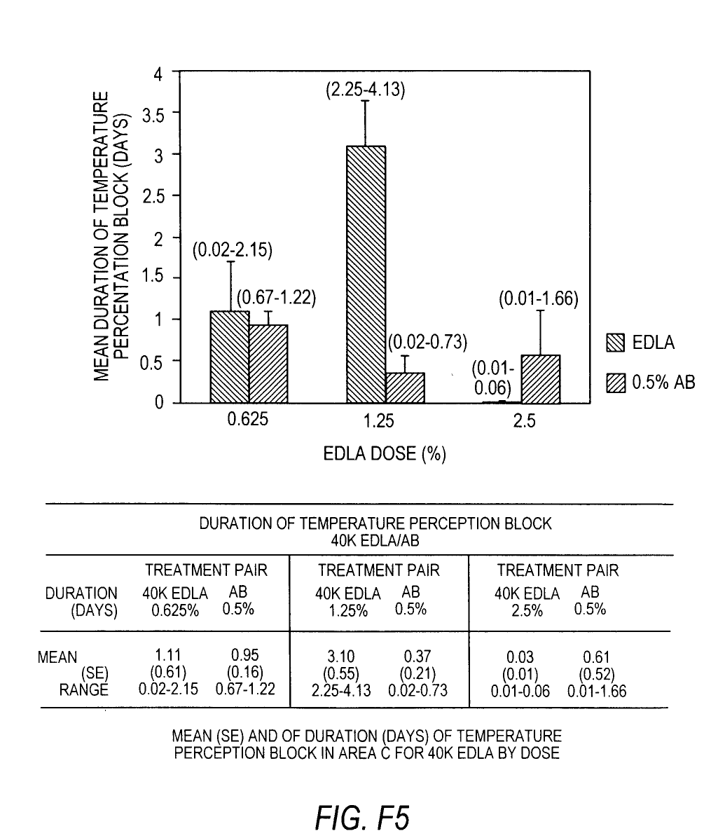

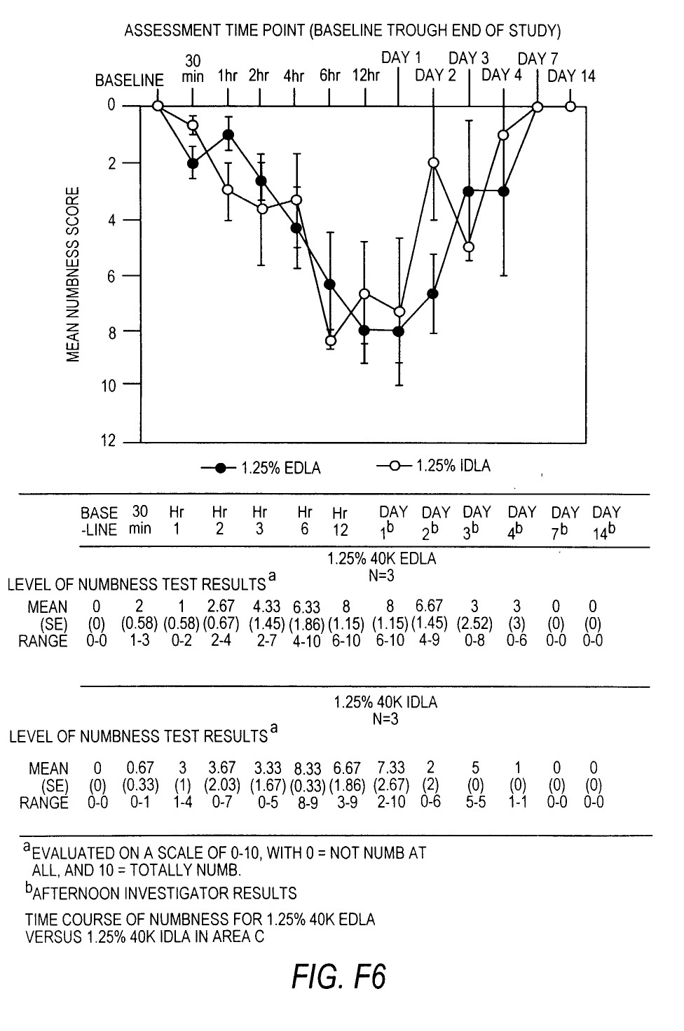

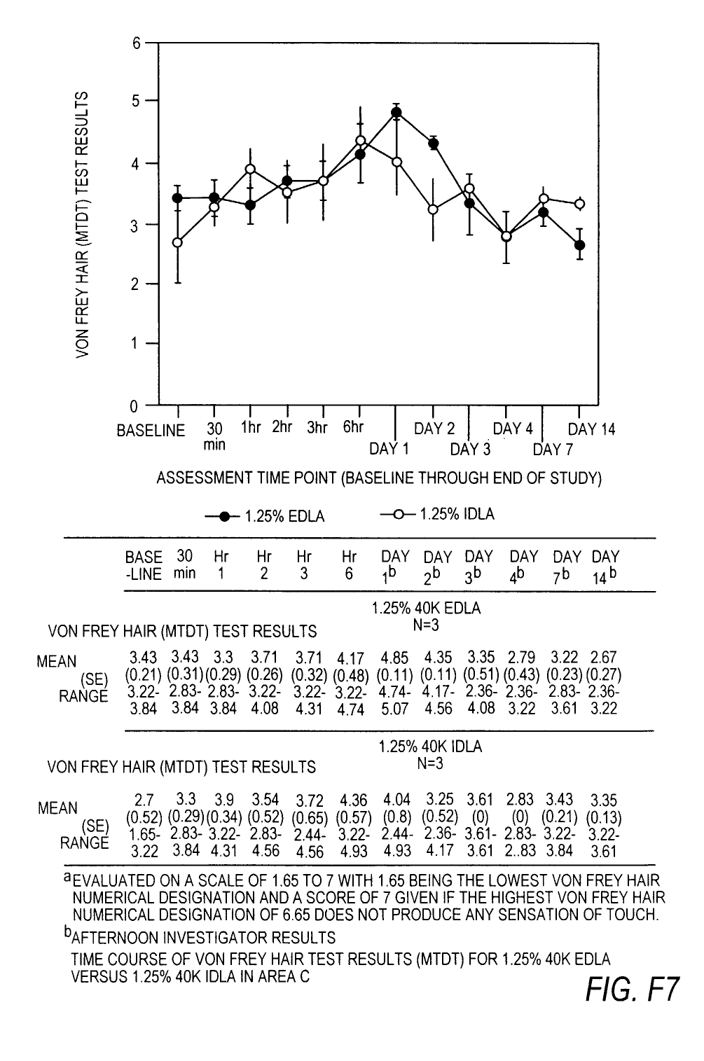

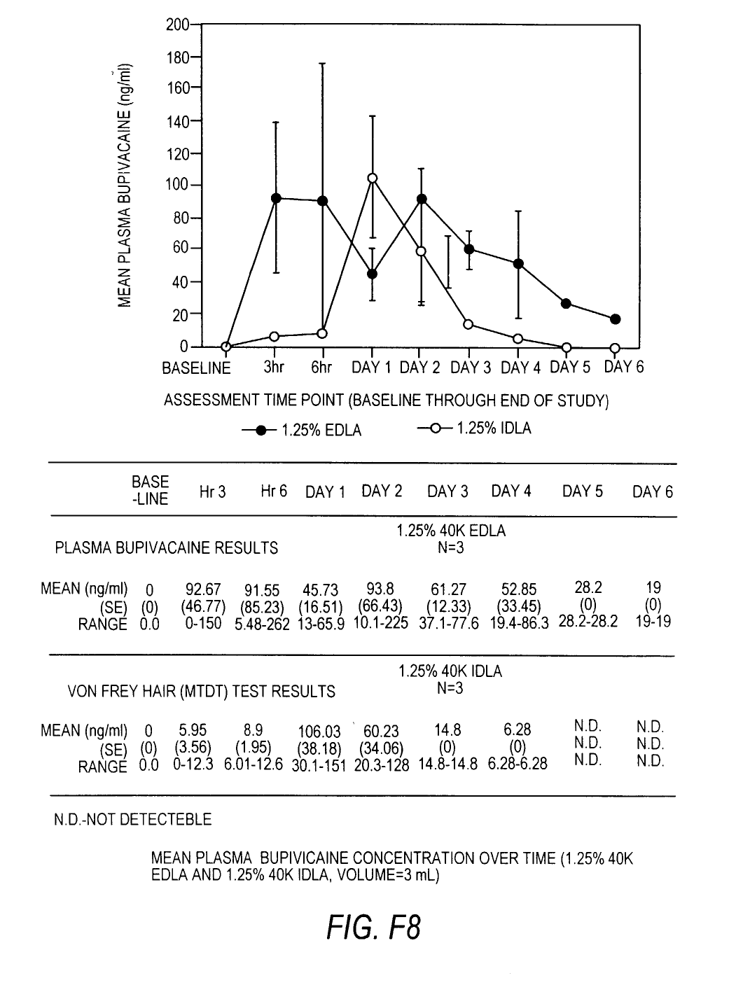

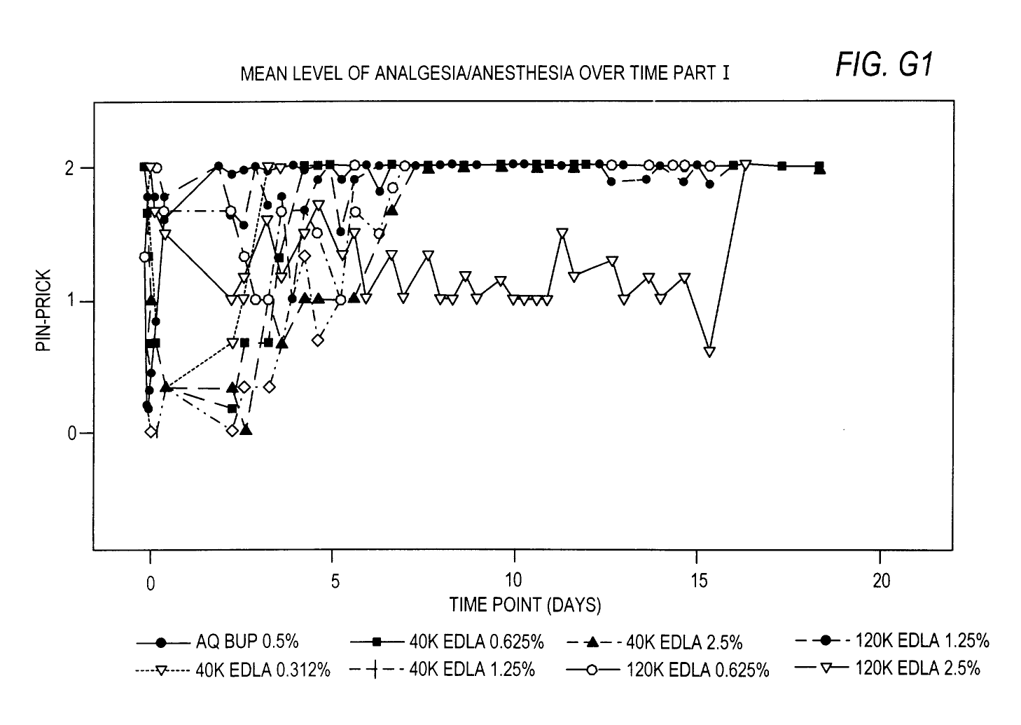

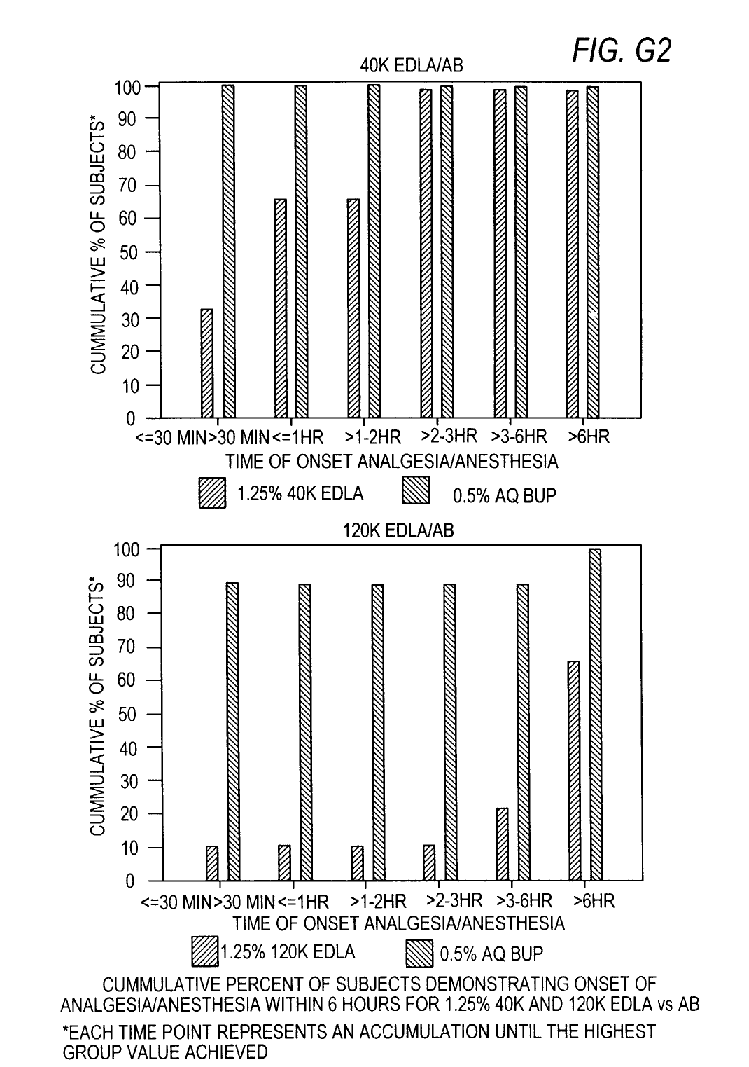

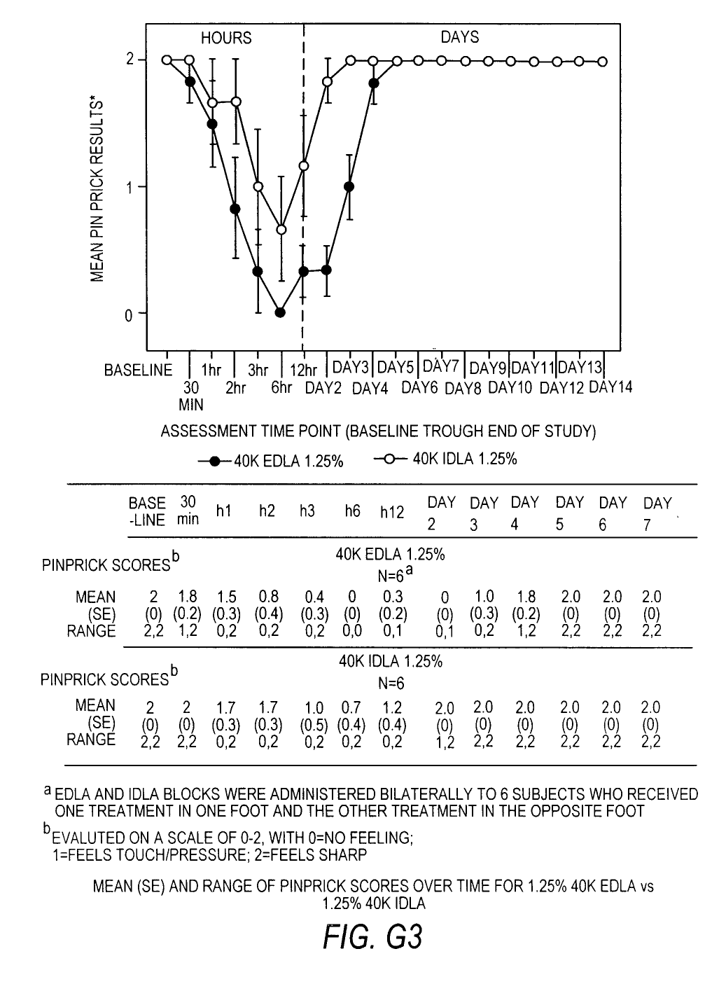

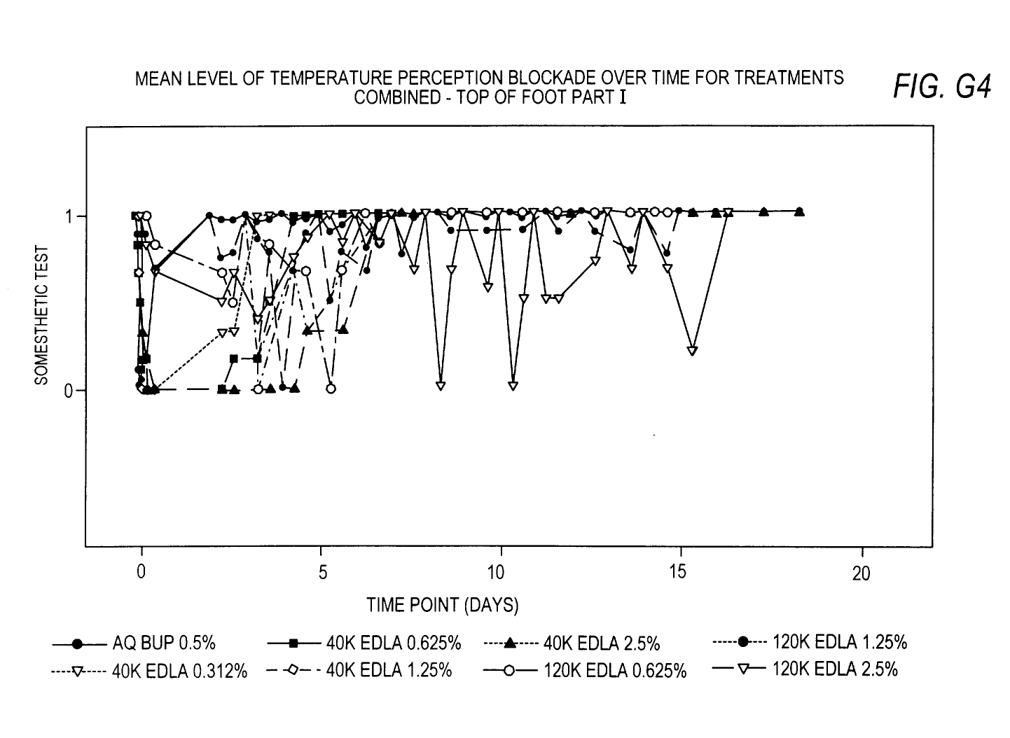

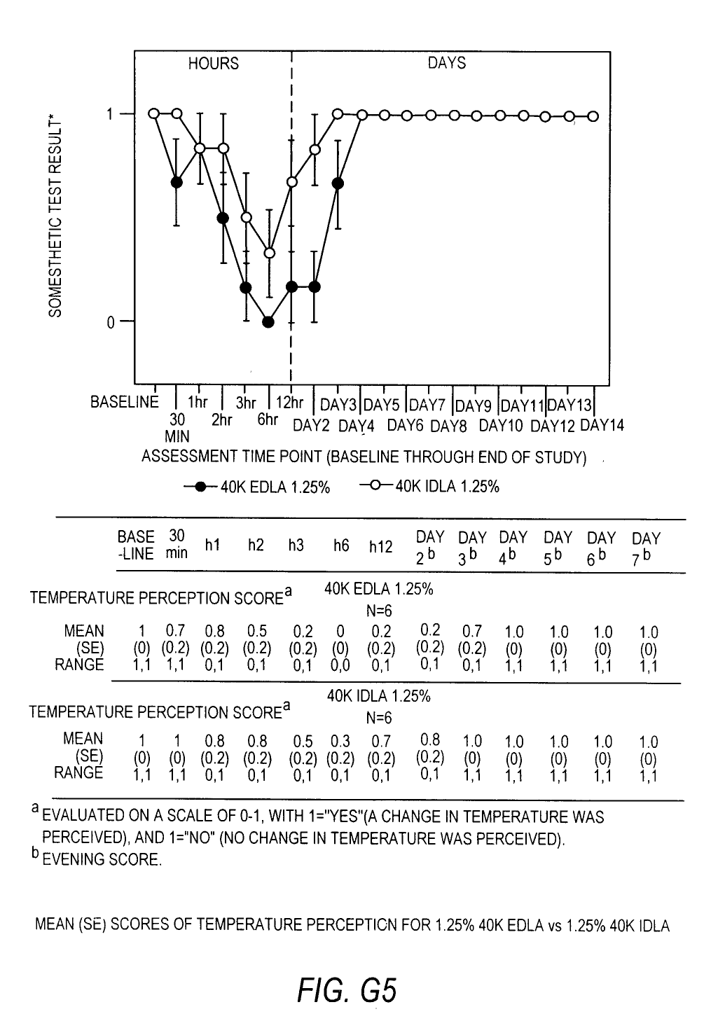

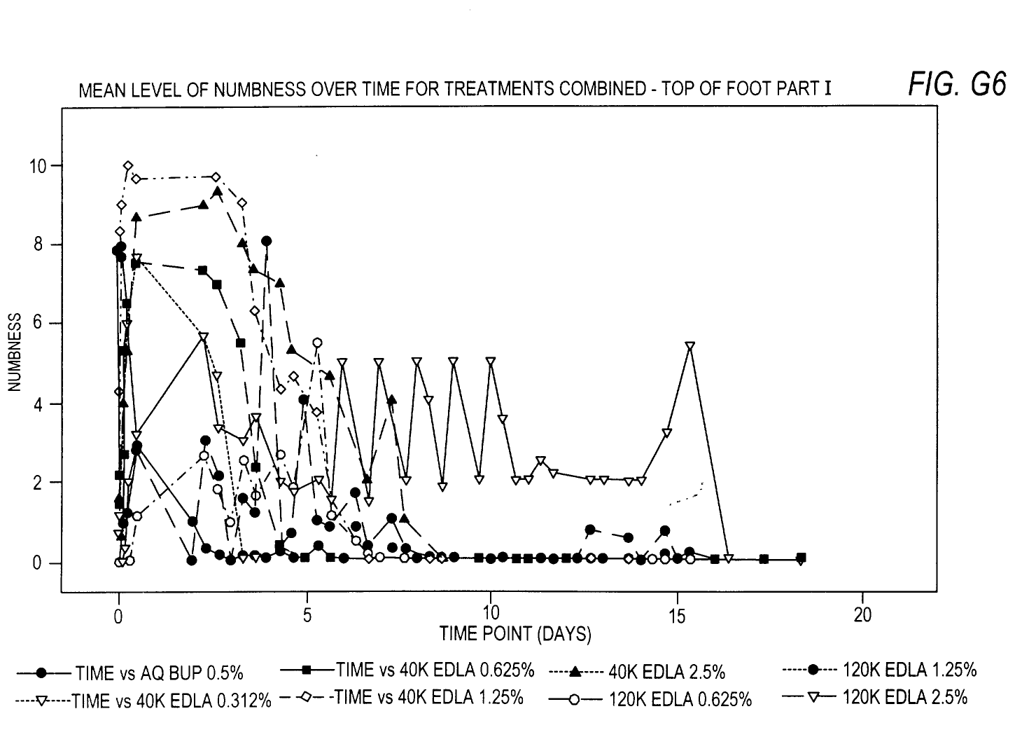

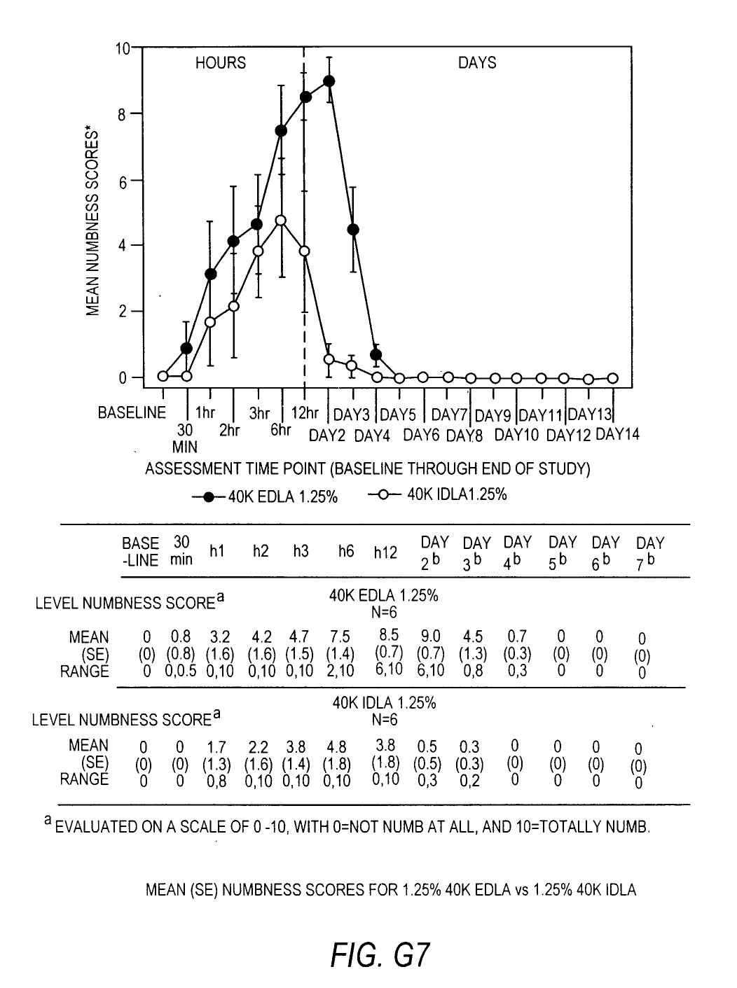

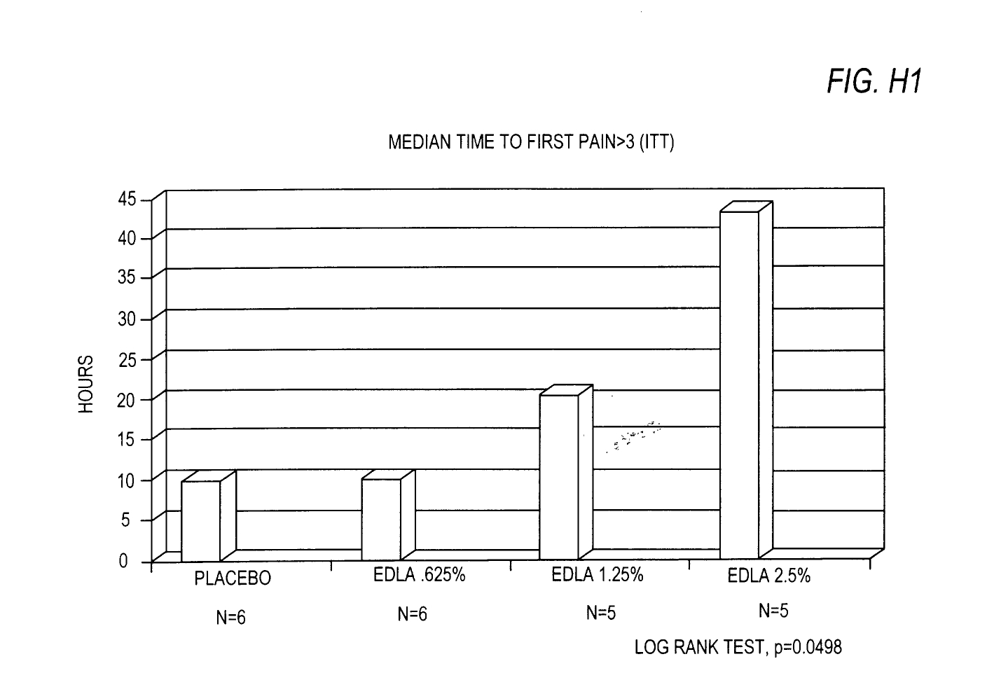

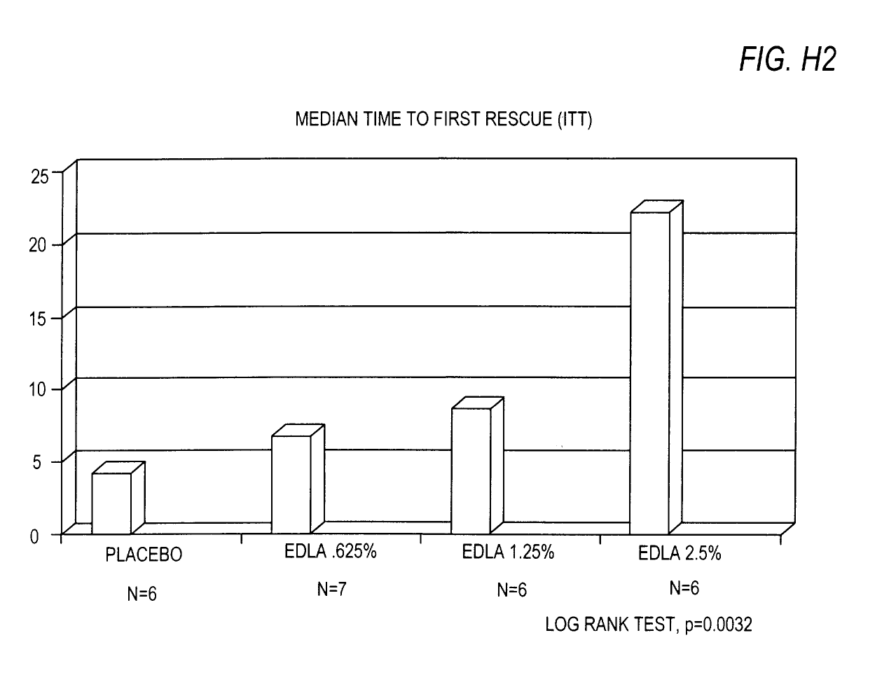

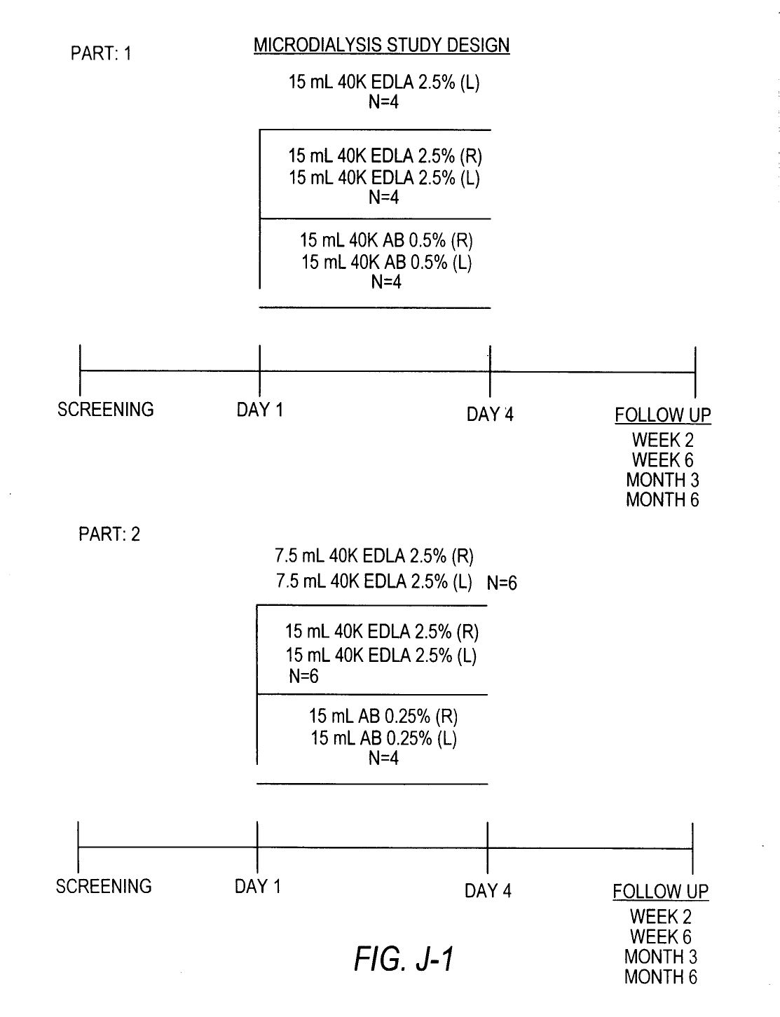

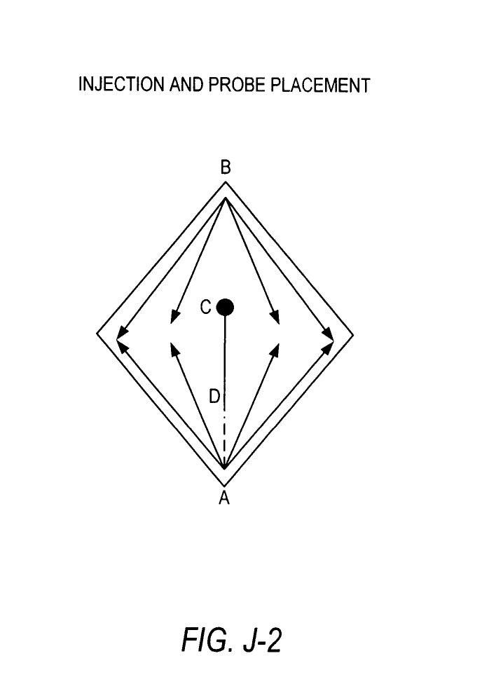

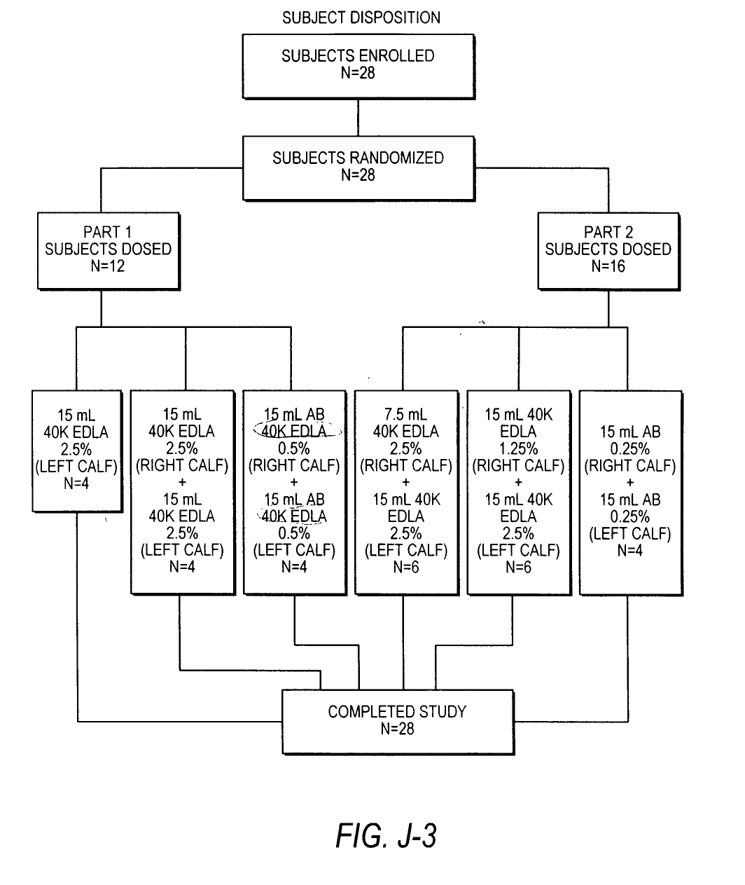

*The average dissolution range for the formulation of Example 2 b is shown in Figure 3.

The preferred dissolution ranges based on the above in-vitro dissolution data is listed below in Tablet 7C:

TABLE 7C

| TIME (Hours) | Percent Release |
|---|---|
| 0 | 0 |
| 1 | From about 13 to about 36 |
| 2 | From about 33 to about 65 |
| 4 | From about 53 to about 87 |
| 8 | From about 72 to about 95 |
| 12 | From about 81 to about 98 |
| 18 | From about 89 to about 100 |
| 24 | From about 94 to about 100 |

### In-Vivo Testing Bupivacaine Microcapsules—Hotplate Model

The in-vivo efficacy of the several formulations was assessed in the rat using hotplate model. The procedure is described in detail in IACUC No 9511-2199. The following paraphrases the procedure.

Male Sprague Dawley rats (Harlan Laboratories, Indianapolis, Ind.) with an average weight of 275 gm were used. The hotplate study consisted of gently holding the body of the animal while the plantar surface of the hind paw was placed on a hotplate heated to 56° C. The baseline latency was determined prior to unilateral injection of local anesthetic around the sciatic nerve of the rat.

For injection of microspheres, the rats were briefly anesthetized with isoflurane to prevent voluntary skeletal muscle contraction during the nerve stimulation procedure. To inject local anesthetics, a sterile 22-gauge STIMEX-4 parylene coated needle (Becton Dickenson, Franklin Lakes, N.J.) was inserted into a 1½ inch 18-gauge needle (Becton Dickenson). (Before use, the 18-gauge needles were cleared of burrs by repeatedly inserting an old STIMEX-4 uncoated needle. Burrs could account for the reports of needle blockage during microsphere injections. The burrs are also cleared to prevent scratching off the pargylene coating. The needles were then packaged and sterilized in an autoclave). The STIMEX-4 needles are coated with parylene to prevent electrical conduction throughout the needle, except at the tip that is un-coated. The fur was depilated at the site of injection, cleansed with sterile cotton swabs saturated with 10% providone iodine and rinsed with cotton swabs saturated with sterile isotonic saline. The surface skin was gently punctured with an 18-gauge needle in order to allow the 18-gauge/STIMEX-4 needle combination to be inserted into the tissue surrounding the nerve. The 18-gauge/STIMEX needle—with attached electrode—was inserted through the skin, between the greater trochanter of the femur and the ischial tuberosity of the pelvis. An electrode was placed on the forepaw. Electrical impulses (Digi Stim II®: <0.9 mA, and 1 Hz) delivered to the sciatic nerve caused hind limb flexion, whereas misplacement of the needle in skeletal or connective tissue failed to stimulate the hind limb. In fact, very close placement led to Digi Stim readings of <0.2 mA. Upon placement of the 18-gauge/STIMEX-4 needle combination, the STIMEX-4 needle was removed while leaving the 18-gauge needle in place near the sciatic nerve. Just prior to injection, the microspheres were briefly suspended by vortexing, and then drawn up into a 1 ml disposable syringe. Syringe volumes were increased an additional 0.07 ml (i.e. 0.6 ml injection volume+0.07 ml=0.67 ml; 0.6 ml delivered), since this represents the dead space of the 18-gauge needle. Thus, the injection of 0.67 ml resulted in 0.6 ml of microspheres deposited around the sciatic nerve.

Physicians and veterinarians routinely use STIMEX needles and nerve stimulators to inject local anesthetic around the nerves in humans and animals. The stimulus is neither painful nor stressful, in that <0.9 mA cannot be detected by humans. Successful injection was evidenced by almost immediate local anesthesia and muscle weakness in the injected hind limb. Animals were housed in plastic cages with bedding to prevent any injury from occurring in the injected paw. Our experience has shown that the integument of the injected paw remains completely intact, with no observed redness, tenderness or sores. The health of the integument is inspected daily. The rats exhibit no stress following the procedure and have no difficulty in obtaining food and water. Test paw withdrawal latencies following drug injection were assessed, and a 12 sec cut-off was imposed to prevent any possible damage that would confound the results. Local anesthesia was quantified as the Hot-Plate Latency (sec).

Time-course studies were analyzed with two-factor repeated measures analysis of variance ANOVA. A significant F-value for the Drug Treatment X Time interaction allowed for post hoc comparisons using the Tukey's test. The Tukey's test allows investigators to make multiple comparisons between any pair of data throughout the time-course.

Dose-response curves were analyzed using least-squares linear regression analysis. In order to calculate effective dose-50 (ED₅₀) values, both baseline and test hot-plate latencies for each rat were converted into percentage of maximum possible effect (% MPE) values. A 12-sec maximum cut-off time was used to prevent damage to the injected paw. % MPE values calculated according to the method of Harris & Pierson (1964) as: % MPE=[(test−control) (12−control)⁻¹]×100. ED₅₀ values with 95% confidence limits were calculated according to the method of Bliss (1967). ED₅₀ calculations were based on linear regression analysis of the scatter-plot of individual rats for the entire dose-response curve. Bliss (1967) developed the following formula to calculate the standard error of the ED₅₀ value. The 95% confidence limits (below, right) are based on the formula by Bliss (1967).

$$S . E . ( {ED}_{50} ) = \vert \frac{s}{m} \vert \sqrt{\frac{1}{N} + \frac{{( ED50 - \overline{x} )}^{2}}{\sum {( {x}_{i} - \overline{x} )}^{2}}} {ED}_{50} \pm t [ S . E . ( {ED}_{50} ) ]$$

$$Calculations$$

### IN-VIVO TESTING OF MICROCAPSULES OF EXAMPLES 1 AND 2

Results

The data are graphically represented for Example 1 in FIG. 2 and for Example 2 in FIG. 3. Two data sets were graphed: mean latency and percent responders. Mean latency represents the average latency of all the animals tested. The error bars represent the standard error of the mean. Latencies over 7 seconds are considered preferred. The percent responders are a measure of the number of animals having latencies greater than 7 seconds as a percent of the total number of animals injected. The efficacy criteria established for this model are mean latency greater than 7 seconds and percent responders 50% or greater.

FIG. 2 shows the mean latency and percent responder data for Example 1, a 72% bupivacaine-loaded 40 kDa microsphere formulation. This formulation, which is identical to Example 2 except that it contains no dexamethasone, shows an anesthetic effect through 24 hours at which time the percent responders drop below 50%.

FIG. 2 shows the mean latency and percent responder data for Example 2, a 72% bupivacaine, 0.04% dexamethasone loaded 40 kDa microcapsule formulation. This formulation shows a significant anesthetic effect lasting through 40 hours (mean latencies greater than 7 seconds; percent responders 50% or greater).

### IN-VIVO TESTING OF 40K, 80K, AND 120K MICROSPHERES

Results

The in-vivo efficacy, as demonstrated by the rat hotplate model latency in seconds, of the three polymers, approximately 40K, 80K and 120K, are listed in Table 8 below:

TABLE 8
In Vivo Efficacy (Rate Hotplate model
Latency measured in Seconds)

| | Ex. | | | | | | |
|---|---|---|---|---|---|---|---|
| Time | | | | | | 3 | 4A |
| (hours) | 2.1 | 2.2 | 2.3 | 2.4 | 2A | 120K | 80K |
| 0 | 1.9 | 1.6 | 2.2 | 2.1 | 2.1 | 2.2 | 1.3 |
| 1 | 11.7 | 10.4 | 12.0 | 11.3 | 12.0 | 5.6 | 1.9 |
| 3 | 12.0 | 10.9 | 11.9 | 12.0 | 11.2 | 5.2 | 1.5 |
| 6 | 8.3 | 10.9 | 10.9 | 12.0 | 9.8 | 4.1 | 1.5 |
| 12 | 9.4 | 11.5 | 9.9 | 11.0 | 9.7 | 3.0 | 2.3 |
| 24 | 8.8 | 9.1 | 10.5 | 11.5 | 9.7 | 1.9 | 1.4 |
| 30 | 8.6 | 9.7 | 11.2 | 9.3 | 8.9 | 2.6 | 1.4 |
| 36 | 7.5 | 6.1 | 9.4 | 6.6 | 7.2 | 2.8 | 2.0 |
| 48 | 8.4 | 7.0 | 7.3 | | 6.2 | 3.2 | 1.8 |
| 54 | 9.8 | 6.8 | 6.3 | | 5.5 | 3.1 | 2.1 |
| 60 | 7.2 | 5.3 | 3.8 | | 3.7 | | 2.0 |
| 72 | 7.8 | 5.6 | 3.2 | | 4.8 | | 2.2 |
| 78 | 8.8 | 6.2 | | | | | |
| 84 | 7.3 | 5.5 | | | | | |
| 96 | 7.7 | | | | | | |
| 102 | | | | | | | |
| 108 | 5.1 | | | | | | |
| 120 | 5.2 | | | | | | |

The data sets for the above table are graphically represented in FIG. 2. Latencies over 7 seconds are preferred, but those at 2 seconds showed a statistically significant effect. A 12 second cutoff was imposed to prevent any possible damage that would confound the results.

### EXAMPLE 6

### Comonomer Ratio

Comonomer ratio is another important property of the polymer which can be used to modify release patterns. Because lactic acid is more hydrophobic than glycolic acid, decreasing the lactic acid content can increase matrix hydrophilicity and increase hydration of the matrix. Although, there is a difference in MWs between these polymers, that alone cannot account for the large difference in release properties of these microspheres.

### EXAMPLE 7

### Hybrid Manipulation of Polymer Molecular Weight and Comonomer Ratio

Polymer MW can be used to manipulate the release profiles. In general, polymers with lower MW produce increased release due to decreased tortuosity and increased flux. Recent work has focused on low MW 50/50 PLGA. There is a significant enhancement of release rate when the low MW 50/50 polymer was used. However, it was difficult to distinguish between the release profiles from the two low MW polymers, MW ˜12K and ˜30K.

These formulations were also tested in vivo (rat hot plate test) at a dose of 50 mg of microspheres per nerve. The closed circles represent the mean latency time in seconds±standard error of the mean. Latency longer than 7 seconds (dashed line) denoted sufficient anesthetic action. The bars represent the percent of animals registering latencies over 7 seconds with the dashed line corresponding to 50%. The 50/50 microspheres produce anesthesia immediately with mean latency remaining above 7 seconds and the number of animals responding above 50% through 48 hours. At 54 and 60 hours, the anesthetic effect is moderated with mean latency falling below 7 seconds and the number of animals responding falling below 50%. This formulation showed excellent onset of action and duration. In contrast, the 75/25 PLGA microspheres showed no anesthetic effect over the period studied (24 hours). Because immediate anesthesia is necessary, this was deemed an unacceptable formulation. This in vivo response is predicted by the in vitro test, where even under aggressive conditions (pH 1.2), the formulation showed only moderate release. These in vivo profiles adequately demonstrate that modification of the comonomer ratio can significantly impact the efficacy of the dosage forms. A change in comonomer ratio from the current 65/35 PLGA will be indicated if the 65/35 low MW PLGA is unable to enhance the release rate.

### EXAMPLE 8

### (End Group)

PLGAs are terminated with either an ester or a free carboxylic acid depending on the nature of the synthesis process. The carboxylic acid-terminated polymers are more hydrophilic in nature due to the ionizable functionality. These polymers hydrate more rapidly leading to more rapid degradation when compared to the less hydrophilic ester-terminated polymers. This effect is more prominent with the lower MW polymers as the contour length to end group ratio is smaller. In the higher MW polymers, changing the end groups has less effect as the physio-chemical properties of the polymer are dominated by the polymer backbone. The increase in degradation reduces the tortuosity and increases diffusion rate. Further, the rapid hydration should result in faster dissolution of bupivacaine and a faster release rate through the polymer matrix.

A related phenomenon which may increase the dissolution of the drug is the microenvironmental effect. This refers to the possibility of a lowered pH environment in the microspheres when using the lower MW hydrophilic PLGA. The lowered pH results from ionization of carboxylic acid residues initially present and constantly generated as this polymer degrades in an aqueous medium. Such a localized acidic environment may aid in dissolution of bupivacaine base and thereby increase its release rate.

### EXAMPLE 9

### Polymer blends

Polymer blending offers another potential possibility for manipulating the release from polymer microspheres containing local anesthetic with or without optional augmenting agent. As previously described, the 50/50 PLGA (MW 10-12K) showed increased release rate, but also were deemed unstable due to crystal formation upon storage. Several polymer blends of 50/50 low MW and 65:35 High MW (polymer used in current process) were evaluated in ratios of 1:1, 3:1, and 9:1 in a attempt to form a stable formulation. The polymers were combined in the organic phase with the active ingredients and the solution filtered. Additional processing steps proceeded as usual.

The 1:1 blend released 66% in 0.5 hr. and about 96% in 24 hr. The 3:1 and 9:1 released drug very rapidly with over 100% (assay variation) released in 0.5 hours. It should be noted that these release conditions are very aggressive. Slightly less aggressive conditions such as higher pH (3.0 or 5.0) may produce a slower release profile providing better correlation with in vivo release. These results demonstrate the utility of the polymer blending to modify the release profile while keeping the drug encapsulated.

The in vivo response of the animals after administration of a 1:1 blend of 50/50 (˜12K) and 65/35 (˜120K) PLGA microspheres was tested. One hour after administration of the formulation, the latency increased to 12 sec (maximum allowable latency). The anesthetic effect continued through 12 hours with latency time around 10 seconds By 24 hours, the mean latency had fallen to about 7 sec and the number of animals responding had dropped below 40%. This would indicate insufficient blocking of pain and therefore this formulation lost effectiveness before 24 hours. At first glance, this profile does not seem to correlate well with the in vitro data. However, closer examination of the in vitro data suggests an explanation for the in vivo behavior. The in vitro data shows very rapid initial release followed by very slow release thereafter, even under the exceedingly acidic (pH 1.2) condition used. In vivo, where pH conditions are closer to neutrality (pH 6.8 to 7.4), the release after the initial release may not have been sufficient to produce anesthesia. Considering the in vivo data this formulation does not produce the desired duration of action.

The in vivo response of the animals after administration of a 3:1 blend of 50/50 (˜12K) and 65/35 (˜120K) PLGA microspheres was tested. In this case, anesthesia occurred rapidly and was maintained through 30 hours. By 36 hours, the mean latency was about 7 sec and the percentage of animals responding was below 50% indicating the diminution of anesthesia. Because the release in vitro was very rapid, little correlation with the in vivo results can be made. However, under the current in vitro release conditions, it appears that very rapid release can produce efficacy for an extended time. This becomes more apparent upon examining the in vivo response after administration of the 9:1 blend of these polymers.

The response profile after administration of the 9:1 blend of these polymers is demonstrated. Once again, the anesthetic effect was realized within 1 hour after administration and continued through 36 hours. By 48 hours, the latency time approached the baseline latency and no animal showed a latency over 7 seconds.

In summary, the in vivo results from the blends indicated that the 1:1 blend was effective for only a day. The 3:1 and 9:1 blends showed efficacy persisting for about two days and diminishing on the third day. These results seem promising in that further experimentation with other ratios of low and high MW polymers could extend the efficacy through 3 days.

### EXAMPLE 10

### Porosinogens

Another possibility in increasing diffusion from the matrix is to increase matrix porosity. Porosinogens can be added to the formulation to facilitate pore formation. A variety of possibilities exist which include inorganic salts and water soluble polymers such as polyethylene glycol.

Inorganic Salts as Porosinogens

Calcium chloride is soluble in ethyl acetate and therefore can be used directly in the organic phase without jeopardizing the inline sterile filtration. EDLA microspheres incorporating 0.01%, 0.025%, 0.05% and 0.1% were made using a solvent extraction technique. The release profiles of these microspheres are depicted in FIG. 13. The release profile at pH 1.2 and 37° C. shows that even the lowest salt concentration of 0.01% release is substantially increased compared to the control microspheres in which 5 mL of EtOH were added without CaCl₂. SEMs of these microspheres, show them to appear spherical and free of crystals. The in vivo response profile (hot-plate test) after administration of the 0.01% CaCl₂ microspheres is shown in FIG. 14. Anesthesia occurs within an hour after administration and continues through 30 hours. Between 36 and 48 hours some marginal anesthesia was evident but by 54 hours it was lost.

In addition to calcium chloride, two other sodium salts (sodium ascorbate and sodium citrate) were used to manufacture with increased porosity. These salts are soluble in ethyl acetate. The release profile is similar to the control microspheres. These salts were incorporated at a very low percent (0.1% and 0.2%) so increasing the concentration five to tenfold to 1% might have a more significant impact on the release kinetics of the system.

The most effective salt used was the CaCl₂ as release was increased even with the low percentage of salt used. Further, because of its solubility in EtOH, the inline sterile filtration of the organic phase would not be compromised.

PEG as a Porosinogen

Polyethylene glycol (PEG) is a water soluble polymer which can be used to induce porosity. PEGs are available in a wide range of MW ensuring versatility in their implementation. Two PEGs (MW 8000 and 4600) were solubilized in EtOH and incorporated in EDLA microspheres as porosinogens. The microspheres have been submitted to PA and in vitro release tests are pending. Drug loading was not compromised by the addition of PEG.

### EXAMPLE 11

### Other Techniques to Increase Release Rate

The salt form of bupivacaine has a better aqueous solubility than the base. This should increase the dissolution rate of the encapsulated drug and thereby increase the release rate. The limitation to using bupivacaine HCl is its limited solubility in ethyl acetate which is the organic solvent in the current manufacturing process.

The rate at which the solvent is removed from the microspheres has been shown to influence the morphology of the microspheres. Removing the solvent at a rapid rate produces microspheres with a very porous internal structure while removing the solvent slowly results in an interior cavity devoid of polymer.

### EXAMPLE 12

### Drug Load

One of the simplest ways to decrease the burst is to decrease the drug loading. The comparative release of two lots of 50/50 low MW PLGA (MW 10-12K) in pH 1.2 buffer at 37° C. was tested. The lower loaded microspheres show a burst of 56% while the 72% loaded microspheres show a burst of 77%. Once again, this release does not mimic in vivo conditions where the release profile could substantially change rendering the difference in burst irrelevant. Nevertheless, the effect of loading on the burst effect is aptly demonstrated in this release profile and may prove useful if it is ascertained that the burst from the low MW polymer is greater than desired.

### Example A

### Sensory Blockade Profile of an Extended Duration Local Anesthetic Administered as a Subcutaneous Injection

A local anesthetic formulation prepared in accordance with Example 2 (EDLA) is administered as a subcutaneous injection on the medial aspect of each calf of human subjects to determine concentrations that provide the desired sensory block. In Part 1 of the study, increasing concentrations are evaluated, up to a maximum concentration of 5.0% for 120K EDLA formulations, and 2.5% for 40K EDLA formulations. Each EDLA formulation is compared with aqueous bupivacaine (0.5%) for reference. Following Part 1, a further comparison study (Part 2) is performed to compare the sensory block afforded by formulations of Example 1 (IDLA) with the sensory block afforded by formulations of EDLA at the same dose (1.25%).

Both the subject and evaluator are blinded as to the treatment being injected in each site for the first four days of evaluation. A randomization schedule designates the calf that is injected with EDLA and the calf that is injected with aqueous bupivacaine. For both sets of experiments, the human subjects receive two injections, either one injection of EDLA into one calf and one injection of aqueous bupivacaine 0.5% into the other calf (Part 1), or one injection of EDLA into one calf and one injection of IDLA into the other calf (Part 2). Subjects are instructed to shave each calf 48 hours prior to the treatment. A 35×60 mm rectangle is drawn on the medial aspect of the right and left calves. A 22-gauge, 1½ inch needle and luer-lock syringe are used to inject a total of 5 mL of study drug in two divided doses of 2.5 mL each: the needle is inserted in opposite comers of the rectangle and 2.5 mL of the drug are injected in a “fan-wise” manner with each needle insertion, saturating the subcutaneous tissue within the rectangle (total volume 5 ml). Each infiltration is administered within 1 hour of study drug preparation as a one-time injection.

The formulations utilized in the study are described in Table Al below, wherein “LMW-EDLA” refers to the formulation of Example 2 utilizing the low molecular weight (40 kD) polymer; “HMW-EDLA” refers to the formulation of Example 2 utilizing the high molecular weight (120 kD) polymer; and “IDLA” refers to the formulation of Example 1 (no dexamethasone) utilizing the low molecular weight (40 kD) polymer: The doses of HMW-EDLA (“120K-EDLA”) are reconstituted and used according to the same procedures described in Table A1.

TABLE A1

| | Concentration | | Strength | |
|---|---|---|---|---|
| Medication | (microspheres) | Dosage Form | Bupivacaine | Dexamethasone |
| LMW-EDLA 0.625%* | 6.25 mg/mL | Microsphere Powder (100 | 4.5 mg/mL | 2.5 mcg/mL |
| | | mg) diluted with 16 mLs of | | |
| | | diluent | | |
| LMW-EDLA 1.25%* | 12.5 mg/mL | Microsphere Powder (100 | 9.0 mg/mL | 5.0 mcg/mL |
| | | mg) diluted with 8 mLs of | | |
| | | diluent | | |
| LMW-EDLA 2.5%* | 25.0 mg/mL | Microsphere Powder (100 | 18.0 mg/mL | 10.0 mcg/mL |
| | | mg) diluted with 4 mLs of | | |
| | | diluent | | |
| LMW-EDLA 5.0%* | 50.0 mg/mL | Microsphere Powder (100 | 36.0 mg/mL | 20.0 mcg/mL |
| | | mg) diluted with 2 mLs of | | |
| | | diluent | | |
| IDLA 1.25%* | 12.5 mg/mL | Microsphere Powder (100 | 9.0 mg/mL | N/A |
| | | mg) diluted with 8 mLs of | | |
| | | diluent | | |
| Aqueous Bupivacaine | 5.0 mg/mL | Bupivacaine 0.5% solution | 5.0 mg/mL | — |

*Percent refers to concentration of microspheres which were approximately 72% loaded with bupivacaine base.

Each study has a total duration of 14 days plus a 6 week safety evaluation and a 6 month long-term safety evaluation.

Efficacy Testing

Testing of Local Anesthetics in human models is often focused on three general areas: MECHANICAL testing (pin prick, von Frey Hairs), THERMAL testing (warm, hot, cool) and TACTILE testing (touch). Multiple testing modalities are used to broadly define the actions of a local anesthetic on a variety of conducting nerves based on size, conduction speed, myelinization, etc. The specifics of testing with these different modalities have been described in the literature, for example, Dahl, et al., Pain, 53:43-51 (1993); Moiniche, et al., Brit. J. of Anaesthesia, 71:201-205 (1993); Pedersen, et al., Anesthesiology, 84(5):1020-1026 (1996); Moiniche, et al., Regional Anesthesia, 18:300-303 (1993); Pedersen, et al., Brit. J. of Anaesthesia, 76(6):806-810 (1996); and, Pedersen, et al., Pain, 74:139-151 (1998), all of which are incorporated by reference herein in their entireties.

In the studies reported herein, the following seven specific modalities are used as a measure of local analgesia, local anesthesia and nerve blockade, making reference to the onset, peak density and duration of effect, based on measured changes in sensory responses. Evaluations are performed at 2, 4, 6, 8, 24, 48, 72 and 96 hours and on days 6, 7 and 8 post-injection.

Mechanical

1) Mechanical Pain Detection Threshold, using progressively stiffer von Frey Hairs;

2) Suprathreshold Pain Response-Mechanical, using von Frey Hair No. 17;

Tactile

3) Mechanical Touch Detection Threshold, using progressively stiffer von Frey Hairs;

Thermal

4) Warm Detection Threshold;

5) Heat Pain Detection Threshold;

6) Suprathreshold Pain Response-Heat; and

7) Cool Detection Threshold.

Each of these modalities and the results of efficacy testing using these modalities is discussed in detail below.

Mechanical and Tactile Testing

MECHANICAL PAIN DETECTION THRESHOLD is defined as the lowest force or number of a von Frey Hair which produces a definite sensation of pain or discomfort, and MECHANICAL TOUCH DETECTION THRESHOLD is defined as the lowest force or number of a von Frey Hair which produces a sensation of touch or pressure. Mechanical Touch Detection Threshold and Mechanical Pain Detection Threshold are determined simultaneously using progressively rigid von Frey Hairs (VFH) (Somedic A/B, Stockholm, Sweden). It was determined that each VFH pressed against a balance until it slightly flexed represents a force which logarithmically increases with each hair, covering a total range of 3 to 402 milliNewtons (mN) (VFH No. 7=3 mN; VFH No. 8=13 mN; VFH No. 9=20 mN; VFH No. 10=39 mN; VFH No. 11=59 mN; VFH No. 12=98 mN; VFH No. 13=128 mN; VFH No. 14=133 mN; VFH No. 15=314 mN; VFH No. 16=350 mN; VFH No. 17=402 mN).

The injected areas are stimulated 8 times with each VFH at a rate of about 2 stimuli per second, starting with VFH No. 7 up to VFH No. 17. The lowest VFH number that is sensed as touch or pressure (Mechanical Touch Detection Threshold) and the lowest number of the hair in which half of the eight stimulations are painful or unpleasant (Mechanical Pain Detection Threshold) are recorded. The procedure is repeated two more times and the median of the three measurements is reported. If VFH No. 17 does not produce the sensation of touch or pressure a Mechanical Touch Detection Threshold value of 18 was assigned. If VFH No. 17 does not produce any pain or discomfort a Mechanical Pain Detection Threshold value of 18 is assigned. SUPRATHRESHOLD PAIN RESPONSE-MECHANICAL to a single von Frey Hair is determined by stimulating the injected areas five times with VFH No. 17 (402 mN). The subject assesses the pain using a VRS scale of 0-10, where zero (0)=no pain and ten=(10) pain as bad as you can imagine.

If one were to run these experiments, one would expect the following data.

Mechanical Pain Detection Threshold

The results for mechanical pain detection threshold testing for Part 1 are tabulated below in Table A2 and illustrated in FIG. A1. As can be seen from Table A2 and FIG. A1, there is a measurable change from baseline in the mechanical pain detection threshold test as early as the two hour testing point. The effect reaches a maximum at from about 6 hours to about 24 hours for some formulations (LMW-EDLA), but in some instances a maximum effect is not observed due to the continued increase in the mechanical pain detection threshold throughout the testing period which is terminated at day eight (HMW-EDLA). The effect continues for some of the formulations tested for at least 8 days, the last time at which efficacy is measured.

TABLE A2
Sensory Evaluations
Mechanical Pain Detection Threshold** For EDLA Over Time up to 8 days

| | 120K | 40K | 120K | 40K | 120K | 40K | 120K | Aq. Bup. |
|---|---|---|---|---|---|---|---|---|
| | 0.625% | 0.625% | 1.25% | 1.25% | 2.5% | 2.5% | 5.0% | 0.5% |
| Baseline | | | | | | | | |
| N | 2 | 6 | 6 | 6 | 6 | 6 | 4 | 18 |
| Mean | 15 | 15.33 | 15 | 14.17 | 16.17 | 15.33 | 15.5 | 16.44 |
| SE* | 2 | 0.95 | 0.73 | 0.98 | 0.48 | 0.42 | 0.65 | 0.22 |
| Median | 15 | 15 | 14.5 | 15 | 16.5 | 15 | 15.5 | 16.5 |
| Min-Max | 13-17 | 12-18 | 13-18 | 10-16 | 14-17 | 14-17 | 14-17 | 15-18 |
| Hour 2 | | | | | | | | |
| N | 2 | 6 | 6 | 6 | 6 | 6 | 4 | 18 |
| Mean | 13.5 | 15.67 | 13.83 | 15.67 | 14.67 | 17.33 | 15.5 | 17.22 |
| SE* | 0.5 | 0.84 | 0.79 | 0.67 | 0.56 | 0.33 | 0.96 | 0.1 |
| Median | 13.5 | 15.5 | 13.5 | 16 | 14.5 | 17.5 | 16 | 17 |
| Min-Max | 13-14 | 13-18 | 12-17 | 13-18 | 13-17 | 16-18 | 13-17 | 17-18 |
| Hour 4 | | | | | | | | |
| N | 2 | 6 | 6 | 6 | 6 | 6 | 4 | 18 |
| Mean | 11.5 | 16 | 13 | 17 | 14.67 | 17.83 | 14.75 | 17.22 |
| SE* | 0.5 | 0.68 | 0.52 | 0.52 | 0.56 | 0.17 | 0.85 | 0.1 |
| Median | 11.5 | 15.5 | 12.5 | 17.5 | 14.5 | 18 | 14.5 | 17 |
| Min-Max | 11-12 | 14-18 | 12-15 | 15-18 | 13-17 | 17-18 | 13-17 | 17-18 |
| Hour 6 | | | | | | | | |
| N | 2 | 6 | 6 | 6 | 6 | 6 | 4 | 18 |
| Mean | 11.5 | 16 | 13.7 | 17 | 14.33 | 18 | 14.75 | 17.22 |
| SE* | 0.5 | 0.77 | 0.83 | 0.68 | 0.92 | 0 | 0.48 | 0.1 |
| Median | 11.5 | 16 | 12 | 18 | 14 | 18 | 14.5 | 17 |
| Min-Max | 11-12 | 14-18 | 12-17 | 14-18 | 12-17 | 18-18 | 14-16 | 17-18 |
| Hour 8 | | | | | | | | |
| N | 2 | 6 | 6 | 6 | 6 | 6 | 4 | 18 |
| Mean | 11.5 | 16 | 13.5 | 18 | 14.17 | 18 | 16 | 17.22 |
| SE* | 0.5 | 0.93 | 0.99 | 0 | 0.95 | 0 | 0.41 | 0.1 |
| Median | 11.5 | 16.5 | 13 | 18 | 13.5 | 18 | 16 | 17 |
| Min-Max | 11-12 | 13-18 | 11-18 | 18-18 | 12-17 | 18-18 | 15-17 | 17-18 |
| Hour 24 | | | | | | | | |
| N | 2 | 6 | 6 | 6 | 6 | 6 | 4 | 18 |
| Mean | 13 | 17.33 | 15.67 | 18 | 16.33 | 18 | 17 | 16.61 |
| SE* | 0 | 0.67 | 0.8 | 0 | 0.67 | 0 | 0 | 0.31 |
| Median | 13 | 18 | 15 | 18 | 17 | 18 | 17 | 17 |
| Min-Max | 13-13 | 14-18 | 14-18 | 18-18 | 13-17 | 18-18 | 17-17 | 13-18 |
| Hour 48 | | | | | | | | |
| N | 2 | 6 | 6 | 6 | 6 | 6 | 4 | 18 |
| Mean | 15 | 16.67 | 16.83 | 18 | 16.33 | 18 | 17 | 15.67 |
| SE* | 1 | 0.61 | 0.65 | 0 | 0.67 | 0 | 0 | 0.43 |
| Median | 15 | 17 | 17.5 | 18 | 17 | 18 | 17 | 16 |
| Min-Max | 14-16 | 14-18 | 14-18 | 18-18 | 13-17 | 18-18 | 17-17 | 11-18 |
| Hour 72 | | | | | | | | |
| N | 2 | 6 | 6 | 6 | 6 | 6 | 4 | 18 |
| Mean | 15.5 | 16 | 16.83 | 17.67 | 16.17 | 18 | 17 | 15.61 |
| SE* | 1.5 | 0.77 | 0.75 | 0.76 | 0.83 | 0 | 0 | 0.56 |
| Median | 15.5 | 16 | 18 | 16 | 17 | 18 | 17 | 16.5 |
| Min-Max | 14-17 | 13-18 | 14-18 | 13-18 | 12-17 | 18-18 | 17-17 | 9-18 |
| Hour 96 | | | | | | | | |
| N | 2 | 6 | 6 | 6 | 6 | 6 | 4 | 18 |
| Mean | 16 | 15.67 | 17.17 | 16.67 | 16.17 | 18 | 17 | 15.61 |
| SE* | 1 | 0.61 | 0.54 | 0.67 | 0.83 | 0 | 0 | 0.51 |
| Median | 16 | 15 | 18 | 17 | 17 | 18 | 17 | 16 |
| Min-Max | 15-17 | 14-18 | 15-18 | 14-18 | 12-17 | 18-18 | 17-17 | 9-18 |
| Day 8 | | | | | | | | |
| N | 2 | 6 | 6 | 6 | 6 | 6 | 4 | 18 |
| Mean | 17 | 15.67 | 17.5 | 16.83 | 15.67 | 15.5 | 17 | 16.17 |
| SE* | 0 | 0.8 | 0.34 | 0.65 | 0.95 | 0.5 | 0 | 0.49 |
| Median | 17 | 15 | 18 | 17.5 | 16.5 | 16 | 17 | 17 |
| Min-Max | 17-17 | 14-18 | 16-18 | 14-18 | 11-17 | 14-17 | 17-17 | 9-18 |

*SE = Standard Error
**Mechanical Pain Detection Threshold—the lowest number of the hair in which half of the 8 stimulations are painful/unpleasant; if VFH No. 17 does not produce any pain or discomfort, a Mechanical Pain Detection Threshold of 18 is recorded

The Mean Mechanical Pain Detection Thresholds over time for 1.25% 40K EDLA and 1.25% 40K IDLA from Part 2 are displayed in FIG. A2.

Onset and Duration of Mechanical Pain Detection Block

Onset of Mechanical Pain Detection Block (using Mechanical Pain Detection Threshold) is the first time at which testing with the von Frey Hair no. 17 does not produce any pain, that is, less than 4 out of 8 applications are painful on at least 2 of 3 repeated tests. The onset of Mechanical Pain Detection Block for 40K EDLA ranges from a mean of 3 to 38 hours and a median of 3 to 16 hours. The higher concentration of 40K EDLA shows a faster mean onset (3 hours) relative to the lowest concentration (38 hours). The onset of Mechanical Pain Detection Block for 1.25% 120K EDLA is 81 and 60 hours (mean and median), which is later than that observed for 1.25% 40K EDLA (5 hours, mean and median).

In Part 2, the 1.25% concentration of 40K EDLA, which is selected as the lowest effective dose in Part 1, is compared to the same concentration of 40K IDLA. The Mean Mechanical Pain Detection Thresholds over time for 1.25% 40K EDLA and 1.25% 40K IDLA are displayed in FIG. A2 and the accompanying table. Onset of Mechanical Pain Detection Block is earlier for 1.25% 40K EDLA (12 and 6 hours, mean and median) compared to 1.25% 40K IDLA (49 and 8 hours, mean and median). The results are shown below in Table A3.

TABLE A3
Onset of Mechanical Pain Detection Block (in hours)^{a, b}
Study Part 1.
120K EDLA

| | Treatment Pair | | Treatment Pair | | Treatment Pair | | Treatment Pair | | | |
|---|---|---|---|---|---|---|---|---|---|---|
| | 120K | | 120K | | 120K | | 120K | | Combined | |
| | EDLA | AB | EDLA | AB | EDLA | AB | EDLA | AB | 120K | |
| | 0.625% | 0.5% | 1.25% | 0.5% | 2.5% | 0.5 | 5% | 0.5% | EDLA | AB |
| | N = 2 | | N = 6 | | N = 6 | | N = 4 | | N = 18 | |
| Mean | 168^{c} | 168 | 81 | 57 | 168 | 168 | 168 | 168 | 139 | 131 |
| SE | 0 | 0 | 28.8 | 35 | 0 | 0 | 0 | 0 | 13.4 | 16.7 |
| Median | 168 | 168 | 60 | 2 | 168 | 168 | 168 | 168 | 168 | 168 |
| Min | 168 | 168 | 8 | 2 | 168 | 168 | 168 | 168 | 8 | 2 |
| Max | 168 | 168 | 168 | 168 | 168 | 168 | 168 | 168 | 168 | 168 |

40K EDLA

| | Treatment Pair | | Treatment Pair | | Treatment Pair | | Combined | |
|---|---|---|---|---|---|---|---|---|
| | 40K EDLA | AB | 40K EDLA | AB | 40K EDLA | AB | 40K | |
| | 0.625% | 0.5% | 1.25% | 0.5% | 2.5% | 0.5% | EDLA | AB |
| | N = 6 | | N = 6 | | N = 6 | | N = 18 | |
| Mean | 38 | 2 | 5 | 2 | 3 | 2 | 16 | 2 |
| SE | 26.3 | 0 | 1.0 | 0 | 0.7 | 0 | 9.1 | 0 |
| Median | 16 | 2 | 5 | 2 | 3 | 2 | 4 | 2 |
| Min | 2 | 2 | 2 | 2 | 2 | 2 | 2 | 2 |
| Max | 168 | 2 | 8 | 2 | 6 | 2 | 168 | 2 |

Study Part 2
40K EDLA/IDLA

Treatment Pair

| | |
|---|---|
| 40K EDLA | 40K IDLA |
| 1.25% | 1.25% |

N = 13)

| | | |
|---|---|---|
| Mean | 12 | 49 |
| SE | 3.9 | 19.6 |
| Median | 6 | 8 |
| Min | 2 | 2 |
| Max | 48 | 168 |

^{a}Mechanical Pain Detection Threshold: the lowest Von Frey Hair (VFH) number that produces a definite sensation of pain or discomfort in 4 of 8 VFH applications up to VFH No. 17.
^{b}Onset of Mechanical Pain Detection Block is defined as the first time at which 4 of 8 applications of VFH, up to No. 17, do not produce pain on at least 2 of 3 tests repeated during a single evaluation.
^{c}Onset at 168 hours = no onset, or failed block

Duration of Mechanical Pain Detection Block is the time from onset of Mechanical Pain Detection Block to offset. Offset of Mechanical Pain Detection Block is the midpoint between the last assessment time point at which VFH No. 17 does not produce pain and the first assessment time point at which a VFH No. 17 or lower does produce pain. Results are shown in Table A4.

TABLE A4
Duration of Mechanical Pain Detection Block^{a}
Study Part 1
120K EDLA/AB

| | Treatment Pair | | Treatment Pair | | Treatment Pair | | Treatment Pair | | | |
|---|---|---|---|---|---|---|---|---|---|---|
| | 120K | | 120K | | 120K | | 120K | | Combined | |
| | EDLA | AB | EDLA | AB | EDLA | AB | EDLA | AB | 120K | |
| | 0.625% | 0.5% | 1.25% | 0.5% | 2.5% | 0.5 | 5% | 0.5% | EDLA | AB |
| | N = 2 | | N = 6 | | N = 6 | | N = 4 | | N = 18 | |
| Mean | 0 | 0 | 86.7 | 44.7 | 0 | 0 | 0 | 0 | 28.9 | 14.9 |
| SE | 0 | 0 | 28.8 | 25.2 | 0 | 0 | 0 | 0 | 13.4 | 9.4 |
| Median | 0 | 0 | 108 | 34 | 0 | 0 | 0 | 0 | 0 | 0 |
| Min | 0 | 0 | 0 | 0 | 0 | 0 | 0 | 0 | 0 | 0 |
| Max | 0 | 0 | 160 | 166 | 0 | 0 | 0 | 0 | 160 | 166 |

Study Part 1
40K EDLA/AB

| | Treatment Pair | | Treatment Pair | | Treatment Pair | | Combined | |
|---|---|---|---|---|---|---|---|---|
| | 40K EDLA | AB | 40K EDLA | AB | 40K EDLA | AB | 40K | |
| | 0.625% | 0.5% | 1.25% | 0.5% | 2.5% | 0.5% | EDLA | AB |
| | N = 6 | | N = 6 | | N = 6 | | N = 18 | |
| Mean | 50.0 | 51.2 | 110.7 | 47.8 | 128.7 | 24.0 | 96.4 | 41 |
| SE | 26.1 | 24.3 | 19.4 | 24.2 | 0.7 | 4.5 | 13.0 | 11.2 |
| Median | 20 | 34 | 104 | 34 | 129 | 24 | 127 | 34 |
| Min | 0 | 1 | 52 | 5 | 126 | 14 | 0 | 1 |
| Max | 166 | 166 | 166 | 166 | 130 | 34 | 166 | 166 |

Study Part 2
40K EDLA/IDLA

Treatment Pair

| | |
|---|---|
| 40K EDLA | 40K IDLA |
| 1.25% | 1.25% |

N = 13)

| | | |
|---|---|---|
| Mean | 80.0 | 42 |
| SE | 13.3 | 14.7 |
| Median | 76 | 12 |
| Min | 6 | 0 |
| Max | 166 | 148 |

^{a}Duration of Mechanical Pain Detection Block, is expressed in hours and is the time from onset of Mechanical Pain Detection Block to offset. Offset of Mechanical Pain Detection Block is the midpoint between the last assessment timepoint at which VFH No. 17 does not produce pain and the first assessment timepoint at which a VFH No. 17 or lower does produce pain.

In Part 1, the duration of Mechanical Pain Detection Block for 40K EDLA ranges from a mean of 50 to 129 hours and a median of 20 to 129 hours. The higher concentration of 40K EDLA shows a longer mean duration (129 hours) relative to the lowest concentration (50 hours). The duration is 80 and 76 hours (mean and median) for 1.25% 40K EDLA, compared to 111 and 104 hours (mean and median) for 1.25% 120K EDLA. The duration of Mechanical Pain Detection Block for aqueous bupivacaine is shorter, as expected (48 hours and 34 hours, mean and median).

In Part 2, the 1.25% concentration of 40K EDLA, which is selected as the lowest effective dose in Part 1, is compared to the same concentration of 40K IDLA. Duration of Mechanical Pain Detection Block is almost twice as long for 1.25% 40K EDLA (80 and 76 hours, mean and median) compared to 1.25% 40K IDLA (42 and 12 hours, mean and median). In addition, the Mean Mechanical Pain Detection Threshold indicates a denser block for 40K EDLA compared to 40K IDLA. As shown in FIG. A2 and Summary Table A10, the maximum increase from the baseline in mechanical pain threshold for 40K EDLA is +2.5, occurring at 24 hours post injection, compared to +1.6 at 8 hours post injection for 40K IDLA, using the mean mechanical pain thresholds.

In summary, the results of the mechanical pain detection threshold tests show that measurable changes in sensory findings occur within 2 hours and an effect that is similar with IDLA and EDLA. The duration of effect is clearly affected by the dexamethasone. This effect ranges from 2-3 days with IDLA, but 4-5 days with EDLA. Duration of block, assessed by the return of Mechanical Pain Detection Threshold to baseline, is slightly later for 40K EDLA than 40K IDLA.

Suprathreshold Pain Response—Mechanical

As discussed above, this test is conducted with a single rigid von Frey Hair that was determined to produce a painful response in subjects. Pain response is determined by stimulating the injected area 5 times with VFH No. 17. Subjects rate pain on the Verbal Rank Scale (VRS) of 0 to 10, with 0=no pain and 10=pain as bad as you can imagine.

For Part 1, the Suprathreshold Pain Response—Mechanical (VRS scores) ranges from a mean baseline of about 1.7 to about 2.5. Sensory block is demonstrated by the change in VRS scores, which shows a decrease from baseline (2.0) after administration of EDLA formulations to about 1 at 2 hours after administration, and a decrease to about 0 to about 0.5 at 24 hours after administration. The effect is observed for at least 8 days after administration. The maximum decrease from baseline occurs for both 40K and 120K EDLA at about 24 hours after administration. The higher concentration of 40K EDLA shows a greater decrease from baseline and a longer duration relative to the lowest concentration. The mean Suprathreshold Pain Response-Mechanical (VRS) scores versus time are shown in Table A5 and FIG. A3.

TABLE A5
Sensory Evaluations
Mean Suprathreshold Pain Response-Mechanical (VRS)** Scores
Over Time up to 8 days

| | 120K | 40K | 120K | 40K | 120K | 40K | 120K | Aq. Bup. |
|---|---|---|---|---|---|---|---|---|
| | 0.625% | 0.625% | 1.25% | 1.25% | 2.5% | 2.5% | 5.0% | 0.5% |
| Baseline | | | | | | | | |
| N | 2 | 6 | 6 | 6 | 6 | 6 | 4 | 18 |
| Mean | 2.5 | 2.5 | 1.67 | 1.83 | 1.57 | 1.83 | 1.75 | 1.72 |
| SE* | 0.5 | 0.85 | 0.56 | 0.4 | 0.34 | 0.54 | 0.48 | 0.3 |
| Median | 2.5 | 2 | 2 | 1.5 | 1 | 2 | 1.5 | 2 |
| Min-Max | 5-5 | 0-5 | 0-3 | 1-3 | 1-3 | 0-4 | 1-3 | 0-4 |
| Hour 2 | | | | | | | | |
| N | 2 | 6 | 6 | 6 | 6 | 6 | 4 | 18 |
| Mean | 3.5 | 2.17 | 2.5 | 1.5 | 2.33 | 1.17 | 2.5 | 0 |
| SE* | 0.5 | 0.91 | 0.62 | 0.43 | 0.71 | 0.31 | 0.65 | 0 |
| Median | 3.5 | 2 | 2.5 | 1.5 | 3 | 1 | 2.5 | 0 |
| Min-Max | 3-4 | 0-6 | 1-5 | 0-3 | 1-4 | 0-2 | 1-4 | 0-0 |
| Hour 4 | | | | | | | | |
| N | 2 | 6 | 6 | 6 | 6 | 6 | 4 | 18 |
| Mean | 4 | 1.67 | 3 | 1.17 | 2.17 | 0.83 | 2.75 | 0 |
| SE* | 1 | 0.92 | 0.68 | 0.4 | 0.6 | 0.31 | 0.48 | 0 |
| Median | 4 | 1 | 3 | 1.5 | 2.5 | 1 | 2.5 | 0 |
| Min-Max | 3-5 | 0-6 | 0-5 | 0-2 | 0-4 | 0-2 | 2-4 | 0-0 |
| Hour 6 | | | | | | | | |
| N | 2 | 6 | 6 | 6 | 6 | 6 | 4 | 18 |
| Mean | 4 | 1.17 | 2.83 | 0.83 | 2.33 | 0.5 | 2.75 | 0 |
| SE* | 1 | 0.65 | 0.95 | 0.31 | 0.71 | 0.22 | 1.03 | 0 |
| Median | 4 | 0.5 | 2 | 1 | 1.5 | 0.5 | 2.5 | 0 |
| Min-Max | 3-5 | 0-4 | 1-7 | 0-2 | 1-5 | 0-1 | 1-5 | 0-0 |
| Hour 8 | | | | | | | | |
| N | 2 | 6 | 6 | 6 | 6 | 6 | 4 | 18 |
| Mean | 4.5 | 1.5 | 2.67 | 0.5 | 1.67 | 0.33 | 3.25 | 0 |
| SE* | 1.5 | 0.96 | 0.88 | 0.22 | 0.56 | 0.21 | 1.03 | 0 |
| Median | 4.5 | 0.5 | 2.5 | 0.5 | 1.5 | 0 | 3.5 | 0 |
| Min-Max | 3-6 | 0-6 | 0-6 | 0-1 | 0-4 | 0-12 | 1-5 | 0-0 |
| Hour 24 | | | | | | | | |
| N | 2 | 6 | 6 | 6 | 6 | 6 | 4 | 18 |
| Mean | 3.5 | 1.17 | 1.33 | 0.17 | 0.17 | 0 | 0 | 0.56 |
| SE* | 0.5 | 0.79 | 0.49 | 0.17 | 0.17 | 0 | 0 | 0.17 |
| Median | 3.5 | 0.5 | 1.5 | 0 | 0 | 0 | 0 | 0 |
| Min-Max | 3-4 | 0-5 | 0-3 | 0-1 | 0-1 | 0-0 | 0-0 | 0-2 |
| Hour 48 | | | | | | | | |
| N | 2 | 6 | 6 | 6 | 6 | 6 | 4 | 18 |
| Mean | 4 | 1 | 0.5 | 0.5 | 0.17 | 0 | 0 | 1 |
| SE* | 1 | 0.82 | 0.34 | 0.34 | 0.17 | 0 | 0 | 0.24 |
| Median | 4 | 0 | 0 | 0 | 0 | 0 | 0 | 1 |
| Min-Max | 3-5 | 0-5 | 0-2 | 0-2 | 0-1 | 0-0 | 0-0 | 0-4 |
| Hour 72 | | | | | | | | |
| N | 2 | 6 | 6 | 6 | 6 | 6 | 4 | 18 |
| Mean | 3 | 1.5 | 0.5 | 0.83 | 0.33 | 0.17 | 0 | 1.5 |
| SE* | 1 | 0.76 | 0.22 | 0.17 | 0.21 | 0.17 | 0 | 0.22 |
| Median | 3 | 1 | 0.5 | 1 | 0 | 0 | 0 | 1 |
| Min-Max | 2-4 | 0-5 | 0-1 | 0-1 | 0-1 | 0-1 | 0-0 | 0-3 |
| Hour 96 | | | | | | | | |
| N | 2 | 6 | 6 | 6 | 6 | 6 | 4 | 18 |
| Mean | 2.5 | 1.5 | 0.33 | 1 | 0.17 | 0.33 | 0 | 1.39 |
| SE* | 0.5 | 0.62 | 0.21 | 0 | 0.17 | 0.21 | 0 | 0.24 |
| Median | 2.5 | 1.5 | 0 | 1 | 0 | 0 | 0 | 1 |
| Min-Max | 2-3 | 0-4 | 0-1 | 1-1 | 0-1 | 0-1 | 0-0 | 0-4 |
| Day 8 | | | | | | | | |
| N | 2 | 6 | 6 | 6 | 6 | 6 | 4 | 18 |
| Mean | 1.5 | 1.5 | 0.33 | 1.17 | 0.5 | 1.5 | 0 | 1.22 |
| SE* | 0.5 | 0.76 | 0.21 | 0.17 | 0.22 | 0.43 | 0 | 0.31 |
| Median | 1.5 | 1 | 0 | 1 | 0.5 | 1.5 | 0 | 1 |
| Min-Max | 1-2 | 0-5 | 0-1 | 1-2 | 0-2 | 0-3 | 0-0 | 0-4 |

*SE = Standard Error
**Suprathreshold Pain Response-Mechanical—pain response to VFH No. 17; subjects assess the pain using a Verbal Rank Scale (VRS) of 0-10, where 0 = no pain and 10 = pain as bad as you can imagine.

In Part 2, the density of blockade of pain response to mechanical stimulation (VFH No. 17), as measured using mean VRS scores from the Suprathreshold Pain Response—Mechanical test, is greater for 40K EDLA versus 40K IDLA, with a maximum decline from baseline of 1.6 versus 1.3, respectively, and a more lasting block over time, for 40K EDLA. The mean Suprathreshold Pain Response-Mechanical (VRS) scores from baseline to Day 8 at each assessment time are shown in FIG. A4.

### Mechanical Touch Detection Threshold

Mechanical Touch Detection Threshold is the lowest VFH number that produced a sensation of touch or pressure in 4 of 8 VFH applications. For Part 1, the Mechanical Touch Detection Threshold ranges from a mean baseline of about 4.5 to about 9.5. Sensory block is demonstrated by the change in thresholds measured, which shows an increase from baseline after administration of EDLA formulations to about 1 at 2 hours after administration, and a increase to about 9 to about 15 at 24 hours after administration. The effect is observed for at least 8 days after administration. The maximum increase from baseline occurs for both 40K and 120K EDLA at about 24 hours after administration. The higher concentration of 40K EDLA shows a greater change from baseline and a longer duration relative to the lowest concentration. The mean Mechanical Touch Detection Thresholds versus time are shown in Table A6 and FIG. A5.

TABLE A6
Sensory Evaluations
Mechanical Touch Detection Threshold** For EDLA Over Time up to 8 days

| | 120K | 40K | 120K | 40K | 120K | 40K | 120K | Aq. Bup. |
|---|---|---|---|---|---|---|---|---|
| | 0.625% | 0.625% | 1.25% | 1.25% | 2.5% | 2.5% | 5.0% | 0.5% |
| Baseline | | | | | | | | |
| N | 2 | 6 | 6 | 6 | 6 | 6 | 4 | 18 |
| Mean | 5.5 | 8.33 | 7.17 | 8.5 | 7.17 | 9.5 | 4.5 | 7.28 |
| SE* | 0.5 | 0.42 | 1.01 | 0.22 | 0.54 | 0.43 | 0.65 | 0.44 |
| Median | 15.5 | 8 | 17.5 | 8.5 | 7 | 9.5 | 4.5 | 7.5 |
| Min-Max | 5-6 | 7-10 | 4-10 | 8-9 | 6-9 | 8-11 | 3-6 | 4-11 |
| Hour 2 | | | | | | | | |
| N | 2 | 6 | 6 | 6 | 6 | 6 | 4 | 18 |
| Mean | 6.5 | 9.33 | 8.33 | 10.67 | 7.5 | 11.67 | 6.5 | 15.17 |
| SE* | 1.5 | 0.67 | 0.99 | 0.8 | 0.34 | 0.42 | 0.87 | 0.35 |
| Median | 6.5 | 9.5 | 8.5 | 10 | 8 | 9.5 | 7 | 15 |
| Min-Max | 5-8 | 7-11 | 4-11 | 9-14 | 6-8 | 8-118 | 4-8 | 12-18 |
| Hour 4 | | | | | | | | |
| N | 2 | 6 | 6 | 6 | 6 | 6 | 4 | 18 |
| Mean | 4.5 | 9.67 | 8.5 | 11.17 | 7.83 | 13.33 | 7.25 | 14.83 |
| SE* | 0.5 | 1.12 | 0.76 | 0.75 | 0.48 | 0.21 | 0.48 | 0.47 |
| Median | 4.5 | 10.5 | 9 | 11.5 | 8 | 13 | 7.5 | 15 |
| Min-Max | 4-5 | 6-12 | 5-10 | 9-13 | 6-9 | 13-14 | 6-8 | 10-18 |
| Hour 6 | | | | | | | | |
| N | 2 | 6 | 6 | 6 | 6 | 6 | 4 | 18 |
| Mean | 5.5 | 10.67 | 8.5 | 12 | 9 | 13.83 | 8.25 | 14.56 |
| SE* | 0.5 | 0.92 | 0.81 | 0.97 | 0.5 | 0.65 | 0.25 | 0.52 |
| Median | 5.5 | 11.5 | 9 | 12 | 8.5 | 13 | 8 | 15 |
| Min-Max | 5-6 | 7-13 | 5-11 | 9-15 | 8-11 | 13-17 | 8-9 | 10-18 |
| Hour 8 | | | | | | | | |
| N | 2 | 6 | 6 | 6 | 6 | 6 | 4 | 18 |
| Mean | 6.5 | 10.67 | 8.83 | 12.17 | 9.17 | 14.17 | 8.25 | 13.94 |
| SE* | 1.5 | 1.15 | 0.65 | 0.65 | 0.6 | 0.83 | 0.25 | 0.57 |
| Median | 6.5 | 11.5 | 9 | 12 | 9 | 14 | 8 | 14.5 |
| Min-Max | 5-8 | 6-14 | 6-11 | 10-14 | 8-12 | 12-18 | 8-9 | 10-17 |
| Hour 24 | | | | | | | | |
| N | 2 | 6 | 6 | 6 | 6 | 6 | 4 | 18 |
| Mean | 5 | 11.5 | 10 | 12.83 | 9.33 | 14.83 | 12.75 | 9.5 |
| SE* | 0 | 0.85 | 1.06 | 0.75 | 1.36 | 0.79 | 1.18 | 0.54 |
| Median | 5 | 12 | 10.5 | 12.5 | 10 | 14.5 | 12 | 9 |
| Min-Max | 5-5 | 9-14 | 6-13 | 11-15 | 3-12 | 13-18 | 11-16 | 6-15 |
| Hour 48 | | | | | | | | |
| N | 2 | 6 | 6 | 6 | 6 | 6 | 4 | 18 |
| Mean | 6 | 11 | 10.67 | 11.5 | 9.17 | 15.5 | 12.5 | 7.83 |
| SE* | 1 | 1.53 | 1.12 | 0.76 | 1.19 | 0.67 | 1.04 | 0.45 |
| Median | 6 | 11 | 11.5 | 11 | 9 | 15 | 12.5 | 8 |
| Min-Max | 5-7 | 6-16 | 6-13 | 10-15 | 4-12 | 14-15 | 10-15 | 3-11 |
| Hour 72 | | | | | | | | |
| N | 2 | 6 | 6 | 6 | 6 | 6 | 4 | 18 |
| Mean | 6 | 9.5 | 9.67 | 11 | 8.83 | 15.33 | 12.25 | 7.17 |
| SE* | 1 | 0.92 | 1.15 | 0.68 | 1.3 | 0.88 | 1.44 | 0.63 |
| Median | 6 | 10 | 10 | 11 | 10 | 14.5 | 11.5 | 7 |
| Min-Max | 5-7 | 6-12 | 6-13 | 9-14 | 4-12 | 13-18 | 10-16 | 3-12 |
| Hour 96 | | | | | | | | |
| N | 2 | 6 | 6 | 6 | 6 | 6 | 4 | 18 |
| Mean | 5 | 9.17 | 10 | 10.17 | 9.33 | 14.17 | 11.75 | 7.22 |
| SE* | 2 | 0.48 | 0.93 | 0.54 | 1.33 | 1.08 | 1.25 | 0.65 |
| Median | 5 | 9 | 10.5 | 11 | 10.5 | 14 | 11.5 | 7.5 |
| Min-Max | 3-7 | 8-11 | 6-12 | 8-11 | 3-12 | 11-18 | 9-15 | 3-13 |
| Day 8 | | | | | | | | |
| N | 2 | 6 | 6 | 6 | 6 | 6 | 4 | 18 |
| Mean | 5.5 | 8.17 | 9.83 | 9.83 | 10 | 11.17 | 11.75 | 6.89 |
| SE* | 2.5 | 0.7 | 1.05 | 0.6 | 1.75 | 0.87 | 0.25 | 0.69 |
| Median | 5.5 | 8 | 10 | 9.5 | 11 | 11 | 12 | 7.5 |
| Min-Max | 3-8 | 6-11 | 6-13 | 8-12 | 3-16 | 8-14 | 11-12 | 3-14 |

*SE = Standard Error
**Mechanical Touch Detection Threshold—the lowest number of the von Frey Hair in which half of the 8 stimulations are sensed as touch or pressure; if VFH No. 17 does not produce any pain or discomfort, a Mechanical Touch Detection Threshold of 18 is recorded.

For Part 2, the mean Mechanical Touch Detection Threshold again indicates a denser block for 40K EDLA compared to 40K IDLA. The maximum increase from mean baseline in pain threshold is +5 for 40K EDLA versus +4 for 40K IDLA, and lasts until Day 2 versus Day 1, for 40K EDLA and IDLA, respectively, using the mean threshold values determined by the test. The mean Mechanical Touch Detection Threshold values over time for all concentrations of EDLA and for 1.25% 40K EDLA and 1.25% 40K IDLA are displayed in FIG. A6.

Thermal Testing

SUPRATHRESHOLD PAIN RESPONSE-HEAT in the injected areas is determined by a stimulus of 45° C. lasting 5 seconds using a computerized 15×25 num thermode (Thermostest, Somedic A/B, Stockholm, Sweden) on the injected areas. The subject assesses pain on a Verbal Rank Scale (VRS) of 0-10, with 0=no pain and 10=pain as bad as you can imagine.

WARM DETECTION THRESHOLD is defined as the lowest increase in temperature from 32° C. perceived, HEAT PAIN DETECTION THRESHOLD is defined as the lowest temperature perceived as painful, and COOL DETECTION THRESHOLD is defined as the lowest decrease in temperature from 32° C. perceived. Warm Detection Threshold, Heat Pain Detection Threshold and Cool Detection Threshold are determined with a computerized Thermostest (Somedic A/B, Stockholm, Sweden) in the injected areas. Subjects are instructed to press a button as soon as the specified sensation is reached. Thermal thresholds are determined from a baseline of 32° C. and increased (Warm Detection Threshold and Heat Pain Detection Threshold) or decreased (Cool Detection Threshold) at a rate of change of 1° C. per second. The upper cut off limit is 52° C. for Warm Detection Threshold and Heat Pain Detection Threshold. The lower cut off limit is 25° C. for Cool Detection Threshold.

Warm Detection Threshold, Heat Pain Detection Threshold and Cool Detection Threshold are calculated as the median of three measurements, with intervals of 10 seconds between each stimulus. If the subject has not perceived warmth or pain at 52° C., the value 53° C. is recorded for Warm Detection Threshold; if the subject has not perceived pain by 52° C., the value of 53° C. is recorded for Heat Pain Detection Threshold; and if the subject has not perceived coolness or pain at 25° C., the value 24° C. is recorded for Cool Detection Threshold.

Suprathreshold Pain Response—Heat

The results for Suprathreshold Pain Response-Heat testing (VRS scores) for Part 1 are tabulated below in Table A7 and FIG. A7. The results show a reduction in VRS scores from a mean baseline of 2.3 to a maximum of 5 before administration to about 0-1 at 24 hours, which is maintained at approximately this low level for the duration of the testing period.

TABLE A7
Sensory Evaluations
Suprathreshold Pain Response-Heat** Over Time up to 8 days

| | 120K | 40K | 120K | 40K | 120K | 40K | 120K | Aq. Bup. |
|---|---|---|---|---|---|---|---|---|
| | 0.625% | 0.625% | 1.25% | 1.25% | 2.5% | 2.5% | 5.0% | 0.5% |
| Baseline | | | | | | | | |
| N | 2 | 6 | 6 | 6 | 6 | 6 | 4 | 18 |
| Mean | 5 | 2.33 | 4.67 | 2.33 | 2.67 | 2.67 | 3.5 | 3.83 |
| SE* | 0 | 0.8 | 0.8 | 0.56 | 0.56 | 0.56 | 0.29 | 0.37 |
| Median | 5 | 1.5 | 5 | 2 | 3 | 3 | 3.5 | 4 |
| Min-Max | 5-5 | 1-6 | 1-6 | 1-4 | 1-5 | 1-4 | 3-4 | 1-7 |
| Hour 2 | | | | | | | | |
| N | 2 | 6 | 6 | 6 | 6 | 6 | 4 | 18 |
| Mean | 5 | 1.83 | 5 | 1.67 | 2.67 | 1.67 | 3 | 0.39 |
| SE* | 1 | 1.08 | 0.63 | 0.49 | 0.61 | 0.49 | 0.71 | 0.18 |
| Median | 5 | 1 | 5.5 | 1.5 | 3 | 1.5 | 2.5 | 0 |
| Min-Max | 4-6 | 0-7 | 2-6 | 0-3 | 1-4 | 0-3 | 2-5 | 0-3 |
| Hour 4 | | | | | | | | |
| N | 2 | 6 | 6 | 6 | 6 | 6 | 4 | 18 |
| Mean | 5 | 1.67 | 5 | 1.17 | 2.17 | 1.5 | 3.75 | 0.33 |
| SE* | 1 | 1.09 | 0.77 | 0.31 | 0.6 | 0.5 | 0.25 | 0.11 |
| Median | 5 | 1 | 5 | 1 | 2.5 | 2 | 4 | 0 |
| Min-Max | 4-6 | 0-7 | 2-7 | 0-2 | 0-4 | 0-3 | 3-4 | 0-1 |
| Hour 6 | | | | | | | | |
| N | 2 | 6 | 6 | 6 | 6 | 6 | 4 | 18 |
| Mean | 5.5 | 1.33 | 5.17 | 1.33 | 2.33 | 1 | 4 | 0.33 |
| SE* | 1.5 | 0.95 | 0.7 | 0.49 | 0.67 | 0.37 | 0.71 | 0.16 |
| Median | 5.5 | 0.5 | 6 | 1.5 | 2 | 1 | 4.5 | 0 |
| Min-Max | 4-7 | 0-6 | 3-7 | 0-3 | 0-5 | 0-2 | 2.5 | 0-2 |
| Hour 8 | | | | | | | | |
| N | 2 | 6 | 6 | 6 | 6 | 6 | 4 | 18 |
| Mean | 5.5 | 1.33 | 5.5 | 1.67 | 1.5 | 1.5 | 4.25 | 0.44 |
| SE* | 1.5 | 0.95 | 0.62 | 0.42 | 0.34 | 0.34 | 1.03 | 0.18 |
| Median | 5.5 | 0.5 | 5.5 | 1 | 2 | 2 | 4.5 | 0 |
| Min-Max | 4-7 | 0-6 | 3-7 | 1-3 | 0-2 | 0-2 | 2-6 | 0-23 |
| Hour 24 | | | | | | | | |
| N | 2 | 6 | 6 | 6 | 6 | 6 | 4 | 18 |
| Mean | 5 | 1 | 4.17 | 0.17 | 1 | 0.33 | 0.75 | 1.94 |
| SE* | 1 | 1 | 0.6 | 0.17 | 0.52 | 0.21 | 0.4 | 0.37 |
| Median | 5 | 0 | 4.5 | 0 | 0.5 | 0 | 0.5 | 2 |
| Min-Max | 4-6 | 0-6 | 2-6 | 0-1 | 0-3 | 0-1 | 0-3 | 0-5 |
| Hour 48 | | | | | | | | |
| N | 2 | 6 | 6 | 6 | 6 | 6 | 4 | 18 |
| Mean | 5 | 1.33 | 3.5 | 0.67 | 0.67 | 0.33 | 0 | 2.78 |
| SE* | 1 | 0.99 | 0.67 | 0.33 | 0.49 | 0.21 | 0 | 0.43 |
| Median | 5 | 0 | 3.5 | 0.5 | 0 | 0 | 0 | 3 |
| Min-Max | 4-6 | 0-6 | 2-5 | 0-2 | 0-3 | 0-1 | 0-0 | 0-7 |
| Hour 72 | | | | | | | | |
| N | 2 | 6 | 6 | 6 | 6 | 6 | 4 | 18 |
| Mean | 4.5 | 1.67 | 2.33 | 0.5 | 0.83 | 0.5 | 0 | 2.61 |
| SE* | 1.5 | 0.95 | 0.67 | 0.22 | 0.48 | 0.5 | 0 | 0.41 |
| Median | 4.5 | 1 | 2.5 | 0.5 | 0.5 | 0 | 0 | 2.5 |
| Min-Max | 3-6 | 0-6 | 0-4 | 0-2 | 0-3 | 0-3 | 0-0 | 0-7 |
| Hour 96 | | | | | | | | |
| N | 2 | 6 | 6 | 6 | 6 | 6 | 4 | 18 |
| Mean | 4 | 1.33 | 1.83 | 0.67 | 0.33 | 0.67 | 0 | 2.56 |
| SE* | 1 | 0.8 | 0.7 | 0.33 | 0.33 | 0.67 | 0 | 0.37 |
| Median | 4 | 0.5 | 1 | 0.5 | 0 | 0 | 0 | 2.5 |
| Min-Max | 3-5 | 0-5 | 0-4 | 0-2 | 0-2 | 0-4 | 0-0 | 0-6 |
| Day 8 | | | | | | | | |
| N | 2 | 6 | 6 | 6 | 6 | 6 | 4 | 18 |
| Mean | 4.5 | 1.83 | 2.17 | 1.17 | 0.5 | 2.17 | 0.25 | 2.56 |
| SE* | 0.5 | 0.75 | 0.54 | 0.4 | 0.34 | 0.54 | 0.25 | 0.35 |
| Median | 4.5 | 1 | 3 | 1.5 | 0 | 2 | 0 | 3 |
| Min-Max | 4.5 | 0-5 | 0-3 | 0-2 | 0-2 | 0-2 | 0-1 | 0-5 |

*SE = Standard Error
**Suprathreshold Pain Response-Heat—pain response to heat determined by a single stimulus of 45 degrees C lasting 5 seconds; pain is assessed by the subject using a Verbal Rank Scale (VRS) of 0-10, where 0 = no pain and 10 = pain as bad as you can imagine.

For Part 2, blockade of Suprathreshold Pain Response-Heat overall is slightly greater for 40K IDLA compared to 40K EDLA, with a maximum decrease in heat pain threshold of 2.2 for 40K IDLA versus 2.0 for 40K EDLA (−2.2 for 40K IDLA and−2.0 for 40K EDLA with respect to baseline). However, the block lasts longer for 40K EDLA, with a −1.5 change from baseline thresholds observed on Day 7 and Day 8 for 40K EDLA compared to Day 3 and Day 4 for 40K IDLA. The decline from baseline for 40K IDLA is −1.1 on Day 8, indicating a faster return to baseline nerve function compared to 40K EDLA (−1.5 on Day 8). The mean Suprathreshold Pain Response-Heat values over time for 1.25% 40K EDLA and IDLA are shown in FIG. A8.

Heat Pain Detection Threshold

Heat Pain Detection Threshold is the lowest temperature perceived as painful when an electrical thermode, set at 32° C. is applied to the injected area. The temperature is increased 1° C. per second up to 52° C. The results for Heat Pain Detection Threshold testing for Part 1 are tabulated below in Table A8 and FIG. A9. The results show that Heat Pain Detection Thresholds, defined as the lowest temperature perceived as painful, increase from a mean baseline of 48 before administration to about 51 at 24 hours, and are maintained at approximately this level for at least 4 days.

TABLE A8
Sensory Evaluations
Heat Pain Detection Threshold** For EDLA Over Time up to 8 days

| | 120K | 40K | 120K | 40K | 120K | 40K | 120K | Aq. Bup. |
|---|---|---|---|---|---|---|---|---|
| | 0.625% | 0.625% | 1.25% | 1.25% | 2.5% | 2.5% | 5.0% | 0.5% |
| Baseline | | | | | | | | |
| N | 2 | 6 | 6 | 6 | 6 | 6 | 4 | 18 |
| Mean | 48.25 | 48.32 | 47.93 | 48 | 48.82 | 46.67 | 47.28 | 48.42 |
| SE* | 0.75 | 0.75 | 0.62 | 19 | 0.61 | 0.62 | 1.22 | 0.3 |
| Median | 48.25 | 48.25 | 47.7 | 49 | 48.8 | 46.8 | 48.15 | 48.75 |
| Min-Max | 47.5-49 | 45.4-51.1 | 45.8-50 | 44.6-50.6 | 46.6-51.1 | 44.6-48.4 | 43.7-49.1 | 44.7-50.2 |
| Hour 2 | | | | | | | | |
| N | 2 | 6 | 6 | 6 | 6 | 6 | 4 | 18 |
| Mean | 48.1 | 48.87 | 47.55 | 48.73 | 49.1 | 48.37 | 47.33 | 50.47 |
| SE* | 1.2 | 0.7 | 0.48 | 0.44 | 0.47 | 0.46 | 0.83 | 0.36 |
| Median | 48.1 | 49.15 | 47.65 | 48.95 | 49.2 | 48.05 | 47.15 | 50.5 |
| Min-Max | 46.9-49.3 | 45.9-50.8 | 46.1-49 | 46.7-49.9 | 47.4-50.5 | 46.8-49.8 | 45.6-49.4 | 48-53 |
| Hour 4 | | | | | | | | |
| N | 2 | 6 | 6 | 6 | 6 | 6 | 4 | 18 |
| Mean | 48 | 49.22 | 47.18 | 49.47 | 48.1 | 49.55 | 46.88 | 50.73 |
| SE* | 0.8 | 1.01 | 0.45 | 0.93 | 0.52 | 0.77 | 0.62 | 0.39 |
| Median | 48 | 50.3 | 47.25 | 50 | 47.7 | 50.3 | 47.05 | 50.65 |
| Min-Max | 47.2-48.8 | 44.5-50.9 | 45.6-48.7 | 45.1-51.8 | 46.9-50.2 | 46.9-51.2 | 45.2-48.2 | 48.1-53 |
| Hour 6 | | | | | | | | |
| N | 2 | 6 | 6 | 6 | 6 | 6 | 4 | 18 |
| Mean | 48.05 | 49.58 | 47.45 | 49.93 | 48.4 | 49.33 | 47.05 | 50.48 |
| SE* | 0.15 | 0.66 | 0.57 | 0.73 | 0.57 | 0.72 | 0.58 | 0.38 |
| Median | 48.05 | 50 | 48.05 | 49.85 | 47.85 | 49.6 | 47.35 | 50.6 |
| Min-Max | 47.9-48.2 | 46.6-50.9 | 45.5-49 | 47-51.9 | 46.9-50.3 | 47.2-51.4 | 45.4-48.1 | 47.3-53 |
| Hour 8 | | | | | | | | |
| N | 2 | 6 | 6 | 6 | 6 | 6 | 4 | 18 |
| Mean | 47.55 | 49.5 | 47.08 | 50.32 | 49.15 | 49.37 | 47.4 | 50.63 |
| SE* | 1.35 | 0.83 | 0.75 | 0.53 | 0.37 | 0.84 | 0.33 | 0.35 |
| Median | 47.55 | 50.3 | 47.9 | 50.35 | 49.1 | 49.8 | 47.3 | 50.55 |
| Min-Max | 46.2-48.9 | 45.4-50.7 | 43.4-48.2 | 48.9-52 | 47.7-50.3 | 46.2-51.2 | 46.8-48.2 | 47.7-53 |
| Hour 24 | | | | | | | | |
| N | 2 | 6 | 6 | 6 | 6 | 6 | 4 | 18 |
| Mean | 47.3 | 51.12 | 48.45 | 50.95 | 49.75 | 50.18 | 49.1 | 49.19 |
| SE* | 0.1 | 1.02 | 0.61 | 0.86 | 0.42 | 0.91 | 0.58 | 0.29 |
| Median | 47.3 | 51.7 | 49.15 | 51.4 | 49.4 | 49.3 | 49.55 | 48.9 |
| Min-Max | 47.2-47.4 | 46.4-53 | 46.1-49.6 | 48.1-53 | 48.8-51.4 | 48.2-53 | 47.4-49.9 | 47.2-51.3 |
| Hour 48 | | | | | | | | |
| N | 2 | 6 | 6 | 6 | 6 | 6 | 4 | 18 |
| Mean | 47.75 | 50.78 | 49.02 | 50.5 | 50.37 | 50.73 | 50.5 | 48.68 |
| SE* | 0.85 | 1.26 | 0.51 | 1.15 | 0.63 | 0.74 | 0.39 | 0.26 |
| Median | 47.75 | 51.85 | 49.4 | 50.8 | 50.2 | 50.1 | 50.45 | 48.6 |
| Min-Max | 46.9-48.6 | 44.9-53 | 46.8-50.4 | 45.4-53 | 48.3-53 | 49.1-53 | 49.6-51.5 | 46.9-50.5 |
| Hour 72 | | | | | | | | |
| N | 2 | 6 | 6 | 6 | 6 | 6 | 4 | 18 |
| Mean | 46.85 | 49.82 | 49.75 | 49.9 | 51.05 | 50.03 | 51.08 | 48.89 |
| SE* | 0.25 | 0.85 | 0.46 | 1.15 | 0.52 | 0.73 | 0.7 | 0.31 |
| Median | 46.85 | 50.75 | 50.15 | 50.75 | 50.95 | 50.25 | 50.8 | 49.1 |
| Min-Max | 46.6-47.1 | 47.1-52.1 | 48.1-50.9 | 44.9-53 | 49.2-53 | 47.3-52.5 | 49.7-53 | 45.9-50.4 |
| Hour 96 | | | | | | | | |
| N | 2 | 6 | 6 | 6 | 6 | 6 | 4 | 18 |
| Mean | 47.45 | 50.3 | 50.13 | 49.63 | 50.97 | 49.55 | 51.33 | 48.94 |
| SE* | 0.35 | 0.62 | 0.5 | 1.15 | 0.51 | 0.91 | 0.68 | 0.23 |
| Median | 47.45 | 50.25 | 49.9 | 49.8 | 50.8 | 50.1 | 51.2 | 48.85 |
| Min-Max | 47.1-47.8 | 48.6-52.1 | 48.5-51.9 | 45-53 | 49.4-53 | 45.9-52.3 | 49.9-53 | 47.4-51.2 |
| Day 8 | | | | | | | | |
| N | 2 | 6 | 6 | 6 | 6 | 6 | 4 | 18 |
| Mean | 49.1 | 48.12 | 49.25 | 49.57 | 50.57 | 47.32 | 50.75 | 49.14 |
| SE* | 0.1 | 1.17 | 0.85 | 0.94 | 0.57 | 0.34 | 1.32 | 0.33 |
| Median | 49.1 | 49.55 | 49.55 | 49.75 | 50.35 | 47.1 | 51.05 | 49.1 |
| Min-Max | 49-49.2 | 44.1-50.4 | 45.6-51.7 | 45.6-52.5 | 48.8-53 | 46.4-48.4 | 47.9-53 | 46.9-53 |

*SE = Standard Error
**Heat Pain Detection Threshold—the lowest increase in temperature from 32 degrees C perceived as painful; if a temperature of 52 is not perceived as painful, a Heat Pain Detection Threshold of 53 is recorded.

For Part 2, onset of Thermal Pain Detection Block (using Heat Pain Detection Threshold) is defined as the first time at which testing of Heat Pain Detection Threshold does not indicate pain using the 52° C. cutoff point on at least 2 of 3 repeated tests. Offset of Heat Pain Detection Block is the midpoint between the last testing point where the Heat Pain Detection Threshold is greater than 52° C. and the first testing point where Heat Pain Detection Threshold is=52° C. Duration of Heat Pain Detection Block is the time from onset of Heat Pain Detection Block to offset of Heat Pain Block. These results are presented in Table A8 and shown in FIG. A10.

Onset and Duration of Heat Pain Detection Block

Onset of Heat Pain Detection Block is defined as the first time point at which 2 of 3 repeated tests for Heat Pain Detection Threshold does not indicate pain detection by the 52° C. cut off point, i.e., the first time point at which a median value of 53° C. is recorded. Subjects are tested through day 7 (168 hours). A mean onset of 168 indicates no effect.

TABLE A9
Onset of Heat Pain Detection Block^{a,b}
Study Part 1.
120K EDLA

| | Treatment Pair | | Treatment Pair | | Treatment Pair | | Treatment Pair | | | |
|---|---|---|---|---|---|---|---|---|---|---|
| | 120K | | 120K | | 120K | | 120K | | Combined | |
| | EDLA | AB | EDLA | AB | EDLA | AB | EDLA | AB | 120K | |
| | 0.625% | 0.5% | 1.25% | 0.5% | 2.5% | 0.5 | 5% | 0.5% | EDLA | AB |
| | N = 2 | | N = 6 | | N = 6 | | N = 4 | | N = 18 | |
| Mean | 168 | 86 | 168 | 113 | 136 | 141 | 126 | 127 | 148 | 122 |
| SE | 0 | 82 | 0 | 34.6 | 21.2 | 27.3 | 24.7 | 41.5 | 9.4 | 17.9 |
| Median | 168 | 86 | 168 | 168 | 168 | 168 | 132 | 168 | 168 | 168 |
| Min | 168 | 4 | 168 | 2 | 48 | 4 | 72 | 2 | 48 | 2 |
| Max | 168 | 168 | 168 | 168 | 168 | 168 | 168 | 168 | 168 | 168 |

Study Part 1
40K EDLA/AB

| | Treatment Pair | | Treatment Pair | | Treatment Pair | | Combined | |
|---|---|---|---|---|---|---|---|---|
| | 40K EDLA | AB | 40K EDLA | AB | 40K EDLA | AB | 40K | |
| | 0.625% | 0.5% | 1.25% | 0.5% | 2.5% | 0.5% | EDLA | AB |
| | N = 6 | | N = 6 | | N = 6 | | N = 18 | |
| Mean | 100 | 144 | 108 | 140 | 120 | 140 | 109 | 142 |
| SE | 30.6 | 24.0 | 28.9 | 27.7 | 30.4 | 27.7 | 16.4 | 14.4 |
| Median | 108 | 168 | 132 | 168 | 168 | 168 | 168 | 168 |
| Min | 24 | 24 | 24 | 2 | 24 | 2 | 24 | 2 |
| Max | 168 | 168 | 168 | 168 | 168 | 168 | 168 | 168 |

Study Part 2
40K EDLA/IDLA

Treatment Pair

| | |
|---|---|
| 40K EDLA | 40K IDLA |
| 1.25% | 1.25% |

N = 13)

| | | |
|---|---|---|
| Mean | 90 | 119 |
| SE | 21.1 | 21.2 |
| Median | 48 | 168 |
| Min | 6 | 2 |
| Max | 168 | 168 |

^{a}Heat Pain Detection Threshold is the lowest temperature perceived as painful when a thermode (32° C.) is applied to the injected area and the temperature is increased 1° C. per second up to 52° C.
^{b}Onset of Heat Pain Detection Block is expressed in hours and is defined as the first time the subject has not indicated pain detection by a 52° C. cut off point on at least 2 of 3 repeated tests. Subjects are tested through day 7 (168 hours). A mean onset of 168 hours indicates no effect.

Heat Pain Detection Block is a more sensitive measure of block because temperature perception is blocked prior to mechanical pain. This is evidenced by a Heat Pain Detection Block for the 2.5% and 5.0% concentrations of 120K EDLA (whereas Mechanical Pain Detection Block shows no effect for both these concentrations), and a sensory block of all 3 concentrations of 40K EDLA. In Part 1 (dose response comparison of EDLA, with aqueous bupivacaine as reference), no onset of Heat Pain Detection Block is observed for the 1.25% concentration of 120K EDLA. Onset of Heat Pain Detection Block for 1.25% 40K EDLA is 108 hours and 132 hours (mean and median). Onset of Heat Pain Detection Block for aqueous bupivacaine is 140 and 168 hours (mean and median). In the absence of an observed effect for the 1.25% concentration of 120K EDLA, 2.5% 120K EDLA is compared to 2.5% 40K EDLA. The onset of Heat Pain Detection Block is faster for 2.5% 40K EDLA compared to 2.5% 120K EDLA (120 and 168 [mean and median] and 136 and 168 hours, respectively).

In Part 2, onset of Heat Pain Detection Block is 90 and 48 hours (mean and median) for 1.25% 40K EDLA, compared to 119 and 168 hours (mean and median) for 1.25% 40K IDLA. Mean Heat Pain Detection Threshold indicates a slightly denser block for 40K EDLA compared to 40K IDLA. The maximum increase from baseline in heat pain threshold is the same (+2) for 40K EDLA and 40K IDLA; however, maximum density of block lasts longer for 40K EDLA (up to 4 days) compared to 40K IDLA (1 day).

Duration of Heat Pain Detection Block

Duration of Heat Pain Detection Block is the time from onset of heat pain block to offset. Offset of Heat Pain Detection Block is the midpoint between the last assessment time point at which the Heat Pain Detection Threshold is greater than 52° C. and the first assessment time point at which Heat Pain Detection Threshold is ≦52° C. Duration of Heat Pain Detection Block is shown in Table A10.

TABLE A10
Duration of Heat Pain Detection Block^{a,b}
Study Part 1.
120K EDLA

| | Treatment Pair | | Treatment Pair | | Treatment Pair | | Treatment Pair | | | |
|---|---|---|---|---|---|---|---|---|---|---|
| | 120K | | 120K | | 120K | | 120K | | Combined | |
| | EDLA | AB | EDLA | AB | EDLA | AB | EDLA | AB | 120K | |
| | 0.625% | 0.5% | 1.25% | 0.5% | 2.5% | 0.5 | 5% | 0.5% | EDLA | AB |
| | N = 2 | | N = 6 | | N = 6 | | N = 4 | | N = 18 | |
| Mean | 0 | 6 | 0 | 2 | 12 | 0.2 | 21 | 0.8 | 8.7 | 1.6 |
| SE | 0 | 6.0 | 0 | 1.6 | 7.6 | 0.2 | 17.2 | 0.8 | 4.6 | 0.8 |
| Median | 0 | 6 | 0 | 0 | 0 | 0 | 6 | 0 | 0 | 0 |
| Min | 0 | 6 | 0 | 0 | 0 | 0 | 0 | 0 | 0 | 0 |
| Max | 0 | 0-12 | 0 | 10 | 36 | 1 | 72 | 3 | 72 | 12 |

Study Part 1
40K EDLA/AB

| | Treatment Pair | | Treatment Pair | | Treatment Pair | | Combined | |
|---|---|---|---|---|---|---|---|---|
| | 40K EDLA | AB | 40K EDLA | AB | 40K EDLA | AB | 40K | |
| | 0.625% | 0.5% | 1.25% | 0.5% | 2.5% | 0.5% | EDLA | AB |
| | N = 6 | | N = 6 | | N = 6 | | N = 18 | |
| Mean | 26 | 2 | 22 | 0.5 | 24 | 0.2 | 24 | 0.9 |
| SE | 17.4 | 2.0 | 10.5 | 0.5 | 17.8 | 0.2 | 8.5 | 0.7 |
| Median | 6 | 0 | 18 | 0 | 0 | 0 | 0 | 0 |
| Min | 1 | 0 | 0 | 0 | 0 | 0 | 0 | 0 |
| Max | 108 | 12 | 60 | 3 | 108 | 1 | 108 | 12 |

Study Part 2
40K EDLA/IDLA

Treatment Pair

| | |
|---|---|
| 40K EDLA | 40K IDLA |
| 1.25% | 1.25% |

N = 13)

| | | |
|---|---|---|
| Mean | 17.6 | 5.5 |
| SE | 9.1 | 4.5 |
| Median | 0 | 0 |
| Min | 0 | 0 |
| Max | 108 | 58 |

^{a}Heat Pain Detection Threshold is defined as the lowest temperature perceived as painful when a thermode (32° C.) is applied to the injected area and the temperature increased 1° C. per second up to 52° C.
^{b}Duration of Heat Pain Detection Block is expressed in hours and is the time from onset of Heat Pain Detection Block to offset. Offset of Heat Pain Detection Threshold is the midpoint between the last assessment timepoint at which the Heat Pain Detection Threshold is greater than 52° C. and the first assessment timepoint at which Heat Pain Detection Threshold is ≦52° C.

In Part 1, duration of Heat Pain Detection Block is 22 and 18 hours (mean and median) for 1.25% 40K EDLA. No Heat Pain Detection Block is observed for 1.25% 120K EDLA. Duration of Heat Pain Detection Block for aqueous bupivacaine is short (0.5 and 0 hours, mean and median, for 1.25% 40K EDLA/aqueous bupivacaine treatment pair).

In the absence of an observed effect for the 1.25% concentration of 120K EDLA, 2.5% 120K EDLA is compared to 2.5% 40K EDLA. The duration of Heat Pain Detection Block is longer for 2.5% 40K EDLA compared to 2.5% 120K EDLA (24 and 0, mean and median, and 12 and 0 hours, respectively).

In Part 2, the 1.25% concentration of 40K EDLA selected as the lowest effective dose in Part 1 is compared to the same concentration of 40K IDLA. Duration of Heat Pain Detection Block is three times as long for 1.25% 40K EDLA (18 and 0 hours, mean and median) compared to 1.25% 40K IDLA (6 and 0 hours, mean and median).

Warm Detection Threshold, Over Time

Warm Detection Threshold is the lowest increase in temperature perceived, starting from a baseline temperature of 32° C. and increasing the temperature in 1° C. increments per second up to 52° C. If the subject does not perceive warmth by 52° C., a value of 53° C. is recorded for Warm Detection Threshold.

In Part 1, the first measurable changes occur within two hours and reach a peak by three hours. An increase in the Warm Detection Threshold is observed from a mean baseline of 40-42 to about 46, occurring within 24 to as late as 72 hours. Warm Detection Threshold values over time are displayed in Table A11 and FIG. A11.

TABLE A11
Sensory Evaluations
Warm Detection Threshold** For EDLA Over Time up to 8 days

| | 120K | 40K | 120K | 40K | 120K | 40K | 120K | Aq. Bup. |
|---|---|---|---|---|---|---|---|---|
| | 0.625% | 0.625% | 1.25% | 1.25% | 2.5% | 2.5% | 5.0% | 0.5% |
| Baseline | | | | | | | | |
| N | 2 | 6 | 6 | 6 | 6 | 6 | 4 | 18 |
| Mean | 41.5 | 42.05 | 41.97 | 40.47 | 42.55 | 41.02 | 39.73 | 41.26 |
| SE* | 2.5 | 0.99 | 1.98 | 0.19 | 1.53 | 0.54 | 0.52 | 0.63 |
| Median | 41.5 | 41.95 | 41.05 | 40.6 | 41.6 | 40.8 | 39.9 | 40.95 |
| Min-Max | 39-44 | 39.3-46.2 | 38.8-46.5 | 39.9-40.9 | 38.9-48.5 | 39.7-43.5 | 38.3-40.8 | 37.5-46.9 |
| Hour 2 | | | | | | | | |
| N | 2 | 6 | 6 | 6 | 6 | 6 | 4 | 18 |
| Mean | 41.5 | 43.43 | 41.35 | 43.2 | 43.32 | 43.7 | 40.68 | 44.81 |
| SE* | 1.15 | 1.29 | 0.72 | 0.74 | 0.97 | 0.77 | 0.44 | 0.63 |
| Median | 41.55 | 44.05 | 40.5 | 43.35 | 43.65 | 43.55 | 40.55 | 44.75 |
| Min-Max | 40.4-42.7 | 38.6-46.2 | 39.7-43.8 | 40.8-45.6 | 40.4-46.9 | 41.3-46 | 39.8-41.8 | 39.7-52 |
| Hour 4 | | | | | | | | |
| N | 2 | 6 | 6 | 6 | 6 | 6 | 4 | 18 |
| Mean | 42.7 | 43.55 | 41.17 | 43.25 | 42.75 | 44.65 | 40.68 | 45.87 |
| SE* | 2 | 1.36 | 0.85 | 1.15 | 1 | 0.95 | 0.38 | 0.5 |
| Median | 42.7 | 44.85 | 40.15 | 43.95 | 41.8 | 44.4 | 40.65 | 45.15 |
| Min-Max | 40.7-44.7 | 39.1-46.5 | 39.4-44 | 39.6-45.9 | 40.6-46.9 | 42.2-47.3 | 39.8-41.6 | 43.7-52 |
| Hour 6 | | | | | | | | |
| N | 2 | 6 | 6 | 6 | 6 | 6 | 4 | 18 |
| Mean | 42.2 | 44.6 | 42.47 | 44.55 | 43.23 | 44.97 | 41.8 | 45.37 |
| SE* | 2 | 1.38 | 0.71 | 0.85 | 0.79 | 0.82 | 0.81 | 0.5 |
| Median | 42.2 | 45.85 | 42.2 | 44.5 | 42.45 | 45.75 | 41.9 | 45.45 |
| Min-Max | 40.2-44.2 | 39.9-46 | 40.7-45.1 | 41.6-47.8 | 41-46.1 | 42.3-47 | 40-43.4 | 42.3-52 |
| Hour 8 | | | | | | | | |
| N | 2 | 6 | 6 | 6 | 6 | 6 | 4 | 18 |
| Mean | 40.15 | 45.23 | 42.82 | 44.63 | 44.3 | 44.9 | 41.63 | 45.39 |
| SE* | 1.15 | 1.19 | 0.83 | 0.81 | 0.75 | 0.54 | 0.39 | 0.49 |
| Median | 40.15 | 46.3 | 43.25 | 44.1 | 44.7 | 45.25 | 41.45 | 45 |
| Min-Max | 39-41.3 | 39.6-47.4 | 40.2-45.4 | 42.7-47.7 | 42-46.2 | 43.2-46.5 | 40.9-42.7 | 43.4-52 |
| Hour 24 | | | | | | | | |
| N | 2 | 6 | 6 | 6 | 6 | 6 | 4 | 18 |
| Mean | 41.7 | 45.98 | 43.77 | 46.23 | 44.52 | 46.45 | 43.28 | 43.33 |
| SE* | 1.6 | 1.28 | 0.88 | 0.99 | 0.7 | 0.66 | 0.37 | 0.52 |
| Median | 41.7 | 44.95 | 44.7 | 46.35 | 44.6 | 46.2 | 43.2 | 42.8 |
| Min-Max | 40.1-43.3 | 40.7-45.4 | 40.2-45.8 | 42.9-49.8 | 42.1-46.3 | 44.3-48.3 | 42.55-44.2 | 39.1-46.5 |
| Hour 48 | | | | | | | | |
| N | 2 | 6 | 6 | 6 | 6 | 6 | 4 | 18 |
| Mean | 41.55 | 45.83 | 43.62 | 44.37 | 44.77 | 46.3 | 45.18 | 42.64 |
| SE* | 2.45 | 1.43 | 1.1 | 1.23 | 0.64 | 0.71 | 0.34 | 0.63 |
| Median | 41.55 | 46.95 | 44.8 | 45.15 | 44.6 | 46.1 | 45.2 | 42.05 |
| Min-Max | 39.1-44 | 39.8-48.7 | 40-46.1 | 39.3-48 | 43.3-46.9 | 43.8-48.4 | 44.5-45.8 | 38.4-47.1 |
| Hour 72 | | | | | | | | |
| N | 2 | 6 | 6 | 6 | 6 | 6 | 4 | 18 |
| Mean | 40.4 | 44.1 | 44.63 | 43.55 | 46.28 | 45.12 | 45 | 41.52 |
| SE* | 1.4 | 0.51 | 1.13 | 1.65 | 0.64 | 0.92 | 1.3 | 0.57 |
| Median | 40.4 | 44.55 | 45.95 | 43.45 | 46.55 | 45.9 | 45.6 | 40.95 |
| Min-Max | 39-41.8 | 42.5-45.3 | 40.6-47.1 | 39.2-49.4 | 43.5-48.1 | 41.2-47.8 | 41.4-47.4 | 38.1-46.7 |
| Hour 96 | | | | | | | | |
| N | 2 | 6 | 6 | 6 | 6 | 6 | 4 | 18 |
| Mean | 41.1 | 43.53 | 44.23 | 43.05 | 44.93 | 44.38 | 44.9 | 41.31 |
| SE* | 1.4 | 0.82 | 1.25 | 1.34 | 1.28 | 1.13 | 0.23 | 0.68 |
| Median | 41.1 | 43.6 | 44.8 | 43 | 45.7 | 45.5 | 44.85 | 41.15 |
| Min-Max | 39.7-42.5 | 41-46.8 | 40.1-48.2 | 39-47.3 | 40.2-48.6 | 40.1-47 | 44.4-45.5 | 37.6-46.6 |
| Day 8 | | | | | | | | |
| N | 2 | 6 | 6 | 6 | 6 | 6 | 4 | 18 |
| Mean | 42.2 | 41.63 | 42.3 | 41.37 | 45.03 | 40.63 | 44.75 | 41.21 |
| SE* | 2.5 | 0.74 | 1.44 | 0.69 | 1.02 | 0.51 | 1.04 | 0.62 |
| Median | 42.2 | 41.55 | 41.5 | 40.75 | 45.4 | 40.5 | 44.45 | 41.05 |
| Min-Max | 39.7-44.7 | 39.5-44.1 | 38.3-47.3 | 40-44.4 | 41.4-47.7 | 39.1-42.2 | 42.6-47.5 | 37.8-46.6 |

*SE = Standard Error
**Warm Detection Threshold—the lowest increase in temperature from 32 degrees C. perceived; if a temperature of 52 is not perceived, a Warm Detection Threshold of 53 is recorded.

The results of Part 2 are shown in FIG. A12. The mean Warm Detection Threshold indicates an equivalent density of block (+4 from baseline) for 40K EDLA and 40K IDLA, with a later onset for 40K EDLA compared to 40K IDLA (24 hours versus 6 hours, respectively), and later offset (Day 2 versus Day 1 for 40K EDLA and 40K IDLA, respectively). The duration of effect is dramatically longer with EDLA.

Cool Detection Threshold

This test is conducted by providing a single exposure to a temperature that is designed to be detectable as cool. These results are shown graphically in FIG. A13.

Conclusions

Overall, a denser and longer sensory block is observed for 40K EDLA compared to 40K IDLA. The density of the effect is measured by the increase from baseline. In mechanical pain and heat pain thresholds, and in mechanical touch and warm thresholds, the density is equivalent or greater for 40K EDLA compared to 40K IDLA, while duration is generally greater for 40K EDLA compared to 40K IDLA, as shown in Table A12 below.

TABLE A12
Summary of Sensory Block Over Time, 1.25% 40K EDLA vs 40K IDLA

| | | | | | |
|---|---|---|---|---|---|
| Mechanical | | Mechanical | | | |
| Paid | Heat Pain | Touch | Warm | Suprathreshold | Suprathreshold |
| Detection | Detection | Detection | Detection | Pain Response- | Pain Response- |
| Threshold | Threshold | Threshold | Threshold | Heat | Mechanical |

Maximum Change from Baseline

| | | | | | | |
|---|---|---|---|---|---|---|
| Density^{a} | | | | | | |
| 40K EDLA | +2.5 | +2 | +5 | +4 | −2.2 | −1.6 |
| 40K IDLA | +1.6 | +2 | +4 | +4 | −2.0 | −1.3 |

Change^{d} from Baseline at 6 hours

Onset^{ab}

| | | | | | | |
|---|---|---|---|---|---|---|
| 40K EDLA | +2 | +1 | +4 | +3 | −1.7 | −1.1 |
| 40K IDLA | +0.9 | +1 | +3 | +4 | −1.9 | −0.9 |

Change^{d} from baseline on Day 4

| | | | | | | |
|---|---|---|---|---|---|---|
| Duration^{c} (days) | | | | | | |
| 40K EDLA | +0.9 | +2 | +2 | +2 | −1.7 | −0.8 |
| 40K IDLA | +0.4 | 0 | +2 | 0 | −1.2 | −0.5 |

^{a}Density of block is expressed as the maximum change from baseline.
^{b}Onset of block is expressed as the change from baseline at 6 hours.
^{c}Duration of block is expressed as the change from baseline on Day 4.
^{d}Change (+/−) = increased pain/detection threshold.

### Example B

### Onset and Duration of Sensory Block After Subcutaneous Infiltration of Long-acting Bupivacaine (120K EDLA)) with Dexamethasone and Aqueous Bupivacaine

A double-blind, randomized, incomplete block design study was performed to evaluate the sensory blockade characteristics (onset and duration of analgesia and anesthesia) and safety profile of 120K EDLA when administered on each arm of human subjects compared to aqueous Bupivacaine (AB). The total duration of the study was 14 days, not including a 14-day screening period, which preceded the first clinic visit and administration of the study drug. Increasing concentrations were evaluated, up to a maximum of 2.5% for 120k EDLA.

Fifteen normal male and female volunteers were enrolled. Subjects reported to the facility on the evening prior to injection for pregnancy and urine drug screens and for baseline evaluation of vital signs and sensory acuity (pinprick, thermal, and tactile tests). The EDLA injections were administered the following morning. The subjects remained at the site for the first 24 hours following injection and were evaluated at specified intervals for onset and degree of sensory blockade as well as for adverse events. Upon discharge, the subjects were instructed to return to the site every day (approximately every 24 hours) for 6 days following the injections and again at day 14 (2 weeks) for a final follow-up evaluation. Sensory blockade testing using pin-prick, thermal, and tactile tests were performed at each visit, and subjects were evaluated to determine injection site reactions, residual or other adverse effects.

Microsphere preparations containing bupivacaine with and without dexamethasone, 120K EDLA 1.25%, and 120K IDLA 1.25%, respectively, and microsphere powder (placebo) were tested. Formulations of 120K EDLA 1.25%, 120K IDLA 1.25%, and microsphere powder (placebo) were administered as a subcutaneous injection (6 mL) on the volar surface of each arm. Each injection site was examined by the investigator every day for 3 days and then every 7 days for 8 weeks and again at 6 months post-injection. Injection sites which developed delayed onset swelling or induration were assessed periodically by measuring the area of induration, along with photographs of the arm with the area of induration outlined. A biopsy was performed if the investigator and sponsor felt the lesion was suitable for biopsy. The swelling or induration tended to be mild, non-painful and resolved without incident.

In evaluating Mechanical Stimuli (pinprick) and tactile stimuli (cotton-ball), assessments were performed at baseline and at the following post-injection times: 15, 30, 45 and 60 minutes, 1.5, 2, 2.5, 3, 6, and 12 hours, and 1, 2, 3, 4, 5, 6, 7, and 14 days. Thermal stimuli evaluations, Warm Detection Threshold and Heat Pain Detection Threshold (WDT and HPDT, respectively) were performed at baseline and at 1, 2, 3, 6 and 12 hours post-injection, and 1, 2, 3, 4, 5, 6, 7, and 14 days post-injection. Suture placement was performed at 3 hours post-injection and used as an additional measure of the depth of sensory block.

In evaluating Degree of Sensory Block, assessments were made by a perceived change in sensation to pinprick. With the subject looking away, the evaluator administered 5 pinpricks to each injected area. The evaluator asked the subject “Did you feel any pin-pricks”? If the subject stated that the pinpricks were felt, they were asked if the pinpricks felt sharp or more like a sensation of touch or pressure.

The response to the pinprick test for sensory block was classified as follows:

| | |
|---|---|
| Anesthesia | 0 = Subject did not feel any pinpricks. |
| Analgesia | 1 = Subject felt pinpricks, but pricks were perceived as touch |
| | or pressure. |
| No Block | 2 = Subject felt sharp pinpricks. |

Analgesia/Anesthesia

Ten of 20 (50%) injections with 120K EDLA resulted in analgesia or anesthesia. Incidence of analgesia/anesthesia varied with concentration. Four of 5 (80%) injections with 120K EDLA 2.5% resulted in analgesia, followed by 3 of 5 (60%) injections with 120K EDLA 0.625%, 2 of 5 (40%) injections with 120K EDLA 0.312% and 1 of 5 (20%) injections with 120K EDLA 1.25%. All subjects (100%) who received 0.25% and 0.5% AB reported analgesia/anesthesia.

Duration of analgesia/anesthesia was defined as the time between onset of analgesia/anesthesia and time when there was a return to sensation of sharpness. Onset and duration of analgesia/anesthesia were both variable. The mean onset of analgesia/anesthesia following injection with 120K EDLA 0.312% was 3.1 hours (range 0.3-6 hours); the mean duration of analgesia/anesthesia was 0 hours (i.e. there was no analgesia/anesthesia at the evaluation time immediately following onset). Injection with 120K EDLA 0.625% resulted in a mean onset of analgesia/anesthesia of 1.2 hours (range 0.3-3 hours). The 120K EDLA 0.625% recipients had the longest mean duration of analgesia/anesthesia (56.3 hours) but also the greatest range of duration (0.0-167.8 hours). Analgesia/anesthesia occurred following one injection with 120K EDLA 1.25% with an onset of 0.5 hours post-injection and a duration of 0.5 hours. Mean onset following injection with 120K EDLA 2.5% was 24.7 hours (range, 0.3-72.0) and the mean duration was 14.9 hours (range, 0.0-48.0).

The mean onset of analgesia/anesthesia following injection of both 0.25% and 0.5% AB was 0.3 hours (range 0.3-0.5 hours). Injections with 0.25% AB resulted in a mean duration of analgesia/anesthesia of 16.3 hours (range, 1.5-47.8), and injections with 0.5% AB demonstrated a mean duration of analgesia/anesthesia of 38.8 hours (range, 2.8-143.8). The results concerning the incidence, onset and duration of analgesia/anesthesia are summarized in Table B 1.

TABLE B1
Onset of Analgesia/Anesthesia

| | AB | | 120K EDLA | | | |
|---|---|---|---|---|---|---|
| Analgesia/ | 0.25% | 0.5% | 0.312% | 0.625% | 1.25% | 2.5% |
| Anesthesia | N = 5 | N = 5 | N = 5 | N = 5 | N = 5 | N = 5 |
| Number (%) of | 5 (100) | 5 (100) | 2 (40) | 3 (60) | 1 (20) | 4 (80) |
| Subjects reporting | | | | | | |
| Analgesia/ | | | | | | |
| Anesthesia | | | | | | |
| Onset^{a} (hours) | 0.3 ± 0.1 | 0.3 ± 0.0 | 3.1 ± 2.9 | 1.2 ± 0.9 | 0.5 ± 0 | 24.7 ± 16.7 |
| mean ± SE | | | | | | |
| Duration^{b} (hours) | 16.3 ± 8.8 | 38.8 ± 26.5 | 0.0 ± 0.0 | 56.3 ± 55.7 | 0.5 ± 0 | 14.9 ± 11.4 |
| mean ± SE | | | | | | |

N = number of subjects who received each treatment (subjects received bilateral injections of 2 different treatments)
^{a}Hours post-injection
^{b}Hours from onset of analgesia/anesthesia to time when there was a return to sensation of sharpness

None of the injections with 120K EDLA resulted in anesthesia, as defined above (Subject did not feel any pinpricks). Three of 5 (60%) injections with 0.25% AB and 4 of 5 (80%) with 0.5% AB led to anesthesia. The mean onset of anesthesia for 0.25% AB injections was 0.7 hours (range, 0.3-1.0 hours) and for 0.5% AB injections mean onset was 0.9 hours (range, 0.3-1.5).

Thermal Stimulation

A determination of Warm Detection Threshold and Heat Pain Detection Threshold was performed using a computerized semiconductor thermode as described in Example A. The detection thresholds were determined from a baseline temperature of 32° C. with a 1° C. per second increase in temperature to a maximum of 52° C. The subjects were instructed to activate a push button when a sensation of warmth was detected (Warm Detection Threshold) and again when the sensation of pain was perceived (Heat Pain Detection Threshold). These values were recorded and the thermode was returned to the baseline temperature. If the cut-off limit of 52° C. was reached and the subject had not indicated pain, the thermode automatically returned to baseline. Subjects who had not perceived warmth or pain by 52° C. were rated as 53° C.

Warm Detection Threshold was defined as the lowest temperature at which warmth was perceived and Heat Pain Detection Threshold as the lowest temperature perceived as painful. Interpretation of “pain” was left to the subject who was instructed to apply the same interpretation throughout the study. Each threshold was calculated as the median of three determinations preformed with intervals of 10 seconds between each stimulation. Mean Warm Detection Threshold and Heat Pain Detection Threshold by timepoint were defined as the average detection temperature at each assessment point. Thermal Pain Block was defined as a Heat Pain Detection Threshold of 53° C.

Thermal Pain Block

Nineteen of 20 (95%) injections with 120K EDLA resulted in thermal pain block and 9 of 10 (90%) injections with AB resulted in thermal pain block. Onset and duration of thermal pain block are summarized in Table B2. The duration of thermal pain block was the time between onset of thermal pain block and the time when there was a return of sensation to pain from heat stimulation (Heat Pain Detection Threshold ≦52° C.). Onset occurred quickly with AB (mean onset was 1.3 hours with 0.25% and 0.6 hours with 0.5%) and with 120K EDLA 2.5% (mean onset, 1.6 hours). Mean onset for the other 120K EDLA treatments was more variable and much later, due to some instances of delayed onset ranging from 3 to 7 days. The mean duration of thermal pain blocks was also variable, ranging from 88.8 hours for 120K EDLA 0.625% to 233.6 hours for 120K EDLA 2.5%. The mean duration of block for 0.25% AB was 161.3 hours and for 0.5% AB was 133.8 hours.

TABLE B2
Onset and Duration of Thermal Pain Block

| | AB | | 120K EDLA | | | |
|---|---|---|---|---|---|---|
| Thermal Pain | 0.25% | 0.5% | 0.312% | 0.625% | 1.25% | 2.5% |
| Block | N = 5 | N = 5 | N = 5 | N = 5 | N = 5 | N = 5 |
| Number (%) of | 4 (80) | 5 (100) | 5 (100) | 5 (100) | 4 (80) | 5 (100) |
| subjects with pain | | | | | | |
| block | | | | | | |
| Onset^{a} (hours) | 1.3 ± 0.6 | 0.6 ± 0.2 | 19.2 ± 14.0 | 50.4 ± 32.3 | 20.5 ± 17.2 | 1.6 ± 0.7 |
| mean ± SE | | | | | | |
| Duration^{b} (hours) | 161.3 ± 68.0 | 133.8 ± 9.8 | 153.6 ± 49.0 | 88.8 ± 37.1 | 177.5 ± 52.9 | 233.6 ± 40.8 |
| mean ± SE | | | | | | |

N = number of subjects who received each treatment (subjects received bilateral injections of 2 different treatments)
^{a}Hours post-injection
^{b}The duration of thermal pain block was the time between onset of thermal pain block Heat Pain Detection Threshold > 52° C.). and time when there was a return of sensation to pain from heat stimulation (Heat Pain Detection Threshold ≦ 52° C.).

The incidence, onset, and duration of altered thermal pain threshold are summarized in Table B3. Altered thermal pain threshold was defined as a Heat Pain Detection Threshold score that is less than 53° C. and differed from the initial value by 3 or more degrees. Sixteen of 20 (80%) injections with 120K EDLA resulted in altered thermal pain threshold. All 10 (100%) injections with AB resulted in altered pain threshold.

The mean onsets of altered pain threshold were longer for the AB treatments (17.2 hours for 0.25% AB and 31.8 hours for 0.5%) than they were for most of the 120K EDLA treatments (2.0, 5.4, and 5.0 hours for 120K EDLA 0.312%, 0.625% and 1.25%, respectively). Onset for 120K EDLA 2.5%, however, was 118 hours, due to one late onset of altered pain threshold with this treatment.

The duration of altered thermal pain detection was the time between onset of thermal pain block and time when Heat Pain Detection Threshold returned to baseline levels. The mean duration of altered pain threshold was 162.8 for 0.25% AB and 205.8 hours for 0.5% AB. For 120K EDLA treatments the mean duration ranged from 108 hours (120K EDLA 2.5%) to 268.2 hours (0.625%).

TABLE B3
Onset and Duration of Altered Thermal Pain Detection

| | AB | | 120K EDLA | | | |
|---|---|---|---|---|---|---|
| Altered Thermal Pain | 0.25% | 0.5% | 0.312% | 0.625% | 1.25% | 2.5% |
| Detection | N = 5 | N = 5 | N = 5 | N = 5 | N = 5 | N = 5 |
| Number (%) of subjects | 5 (100) | 5 (100) | 5 (100) | 5 (100) | 3 (60) | 3 (60) |
| with altered thermal pain | | | | | | |
| detection | | | | | | |
| Onset^{a} (hours) | 17.2 ± 8.8 | 31.8 ± 16.5 | 2.0 ± 1.0 | 5.4 ± 1.9 | 5.0 ± 3.5 | 118.0 ± 109.0 |
| mean ± SE | | | | | | |
| Duration^{b} (hours) | 162.8 ± 67.5 | 205.8 ± 57.0 | 209.2 ± 55.7 | 268.2 ± 61.6 | 143.0 ± 93.6 | 108.0 ± 108.0 |
| mean ± SE | | | | | | |

N = number of subjects who received each treatment (subjects received bilateral injections of 2 different treatments)
^{a}Hours postinjection
^{b}The duration of altered pain threshold detection was the time between onset of thermal pain block and time when Heat Pain Detection Threshold returned to baseline levels.

Heat Block

Subjects were considered to have heat block if their Warm Detection Threshold reached 53° C. Three of 20 (15%) subjects who received 120K EDLA experienced heat block. Six of 10 (60%) subjects who received AB demonstrated heat block, as shown in Table B4. Onset of heat block for the two AB treatment groups was relatively quick: 1.0 hours for 0.25% AB and 1.5 hours for 0.5% AB. For subjects who received 120K EDLA, the mean onset of heat block was later: 12 hours for 120K EDLA 0.625% and 18 hours for 120K EDLA 2.5%. The duration of heat block was similar for the AB treatment groups (8 hours for 0.25% AB and 6.5 hours for 0.5% AB) but vastly different for the 120K EDLA groups (108 hours for 120K EDLA 0.625% and no duration for the 120K EDLA 2.5% group).

TABLE B4
Onset and Duration of Heat Block

| | AB | | 120K EDLA | | | |
|---|---|---|---|---|---|---|
| | 0.25% | 0.5% | 0.312% | 0.625% | 1.25% | 2.5% |
| Heat Block | N = 5 | N = 5 | N = 5 | N = 5 | N = 5 | N = 5 |
| Number (%) of | 2 (40) | 4 (80) | 0 | 1 (20) | 0 | 2 (40) |
| subjects with heat | | | | | | |
| block | | | | | | |
| Onset^{a} (hours) | 1.0 ± 0.0 | 1.5 ± 0.3 | — | 12.0 | — | 18.0 ± 6.0 |
| mean ± SE | | | | | | |
| Duration^{b} (hours) | 8.0 ± 3.0 | 6.5 ± 2.7 | — | 108.0 | — | 0.0 ± 0.0 |
| mean ± SE | | | | | | |

N = number of subjects who received each treatment (subjects received bilateral injections of 2 different treatments)
^{a}Hours post-injection
^{b}The duration of heat block was the length of time that Warm Detection Threshold remained at 53° C.

Tactile Perception Block

In evaluating Tactile Stimuli (Cotton-ball), sensibility to touch was evaluated by the subject's ability to perceive “touch” when a cotton-ball was lightly brushed on the skin in the injected area. The subject looked away while the evaluator tested the area with a cotton-ball. The subject was asked, “Tell me if you feel something touching your arm”? The subject responded with “yes” or no

Only 2 of 20 (10%) treatments with 120K EDLA resulted in tactile block but 7 of 10 (70%) treatments with AB resulted in tactile perception block, as shown in Table B5. The mean onset of tactile perception block for the AB injections was faster than for thermal pain block or heat block: 0.4 hours for 0.25% AB and 0.3 hours for 0.5% AB. Following injections with 120K EDLA, the mean onset of tactile perception block was short: 2.5 hours for both 120K EDLA 0.625% and 120K EDLA 2.5%, the only treatment groups that experienced tactile perception block.

The mean duration of tactile block was 2.2 hours for 0.25% AB and 7.8 hours for 0.5% AB. Tactile block resulting from treatment with 120K EDLA 2.5% had no duration, and treatment with 120K EDLA 0.625% resulted in tactile block 3.5 hours in duration.

TABLE B5
Onset and Duration of Tactile Block

| | AB | | 120K EDLA | | | |
|---|---|---|---|---|---|---|
| | 0.25% | 0.5% | 0.312% | 0.625% | 1.25% | 2.5% |
| Tactile Block | N = 5 | N = 5 | N = 5 | N = 5 | N = 5 | N = 5 |
| Number (%) of | 4 (80) | 3 (60) | 0 | 1 (20) | 0 | 1 (20) |
| subjects with tactile | | | | | | |
| block | | | | | | |
| Onset^{a} (hours) | 0.4 ± 0.1 | 0.3 ± 0.0 | — | 2.5 | — | 2.5 |
| mean ± SE | | | | | | |
| Duration^{b} (hours) | 2.2 ± 1.3 | 7.8 ± 2.0 | — | 3.5 | — | 0.0 |
| mean ± SE | | | | | | |

N = number of subjects who received each treatment (subjects received bilateral injections of 2 different treatments)
^{a}Hours postinjection
^{b}The duration of tactile perception block was the length of time that the subject was unable to feel the touch of the cotton-ball in the injected area

Pain on Suture Placement

Pain on Suture Placement was used as an additional method to evaluate the depth of sensory block. A 4-0 silk suture was placed full thickness and incorporated some subcutaneous tissue in the injected area. The subject was asked to rate the pain on insertion of the suture using an 11-point Verbal Rank Scale with 0=“no pain” and 10=“pain as bad as you can imagine.” All of the subjects who received 120K EDLA reported at least some pain on suture placement, as shown in Table B6. Subjects who received 120K EDLA 0.625% reported the most (mean, 6.6) and those who received 120K EDLA 2.5% reported the least (mean 3.2). None of the subjects who received AB, either 0.25% or 0.5%, reported pain on suture placement.

TABLE B6
Pain on Suture Placement

| | AB | | 120K EDLA | | | |
|---|---|---|---|---|---|---|
| Pain on Suture | 0.25% | 0.5% | 0.312% | 0.625% | 1.25% | 2.5% |
| Placement | N = 5 | N = 5 | N = 5 | N = 5 | N = 5 | N = 5 |
| Number (%) of | 0 | 0 | 5 (100) | 5 (100) | 5 (100) | 5 (100) |
| subjects with pain on | | | | | | |
| suture placement | | | | | | |
| Pain on placement^{a} | — | — | 4.4 ± 1.6 | 6.6 ± 1.2 | 4.6 ± 1.3 | 3.2 ± 1.3 |
| mean ± SE | | | | | | |

N = number of subjects who received each treatment
^{a}Rated on an 11-point scale in which 0 = “no pain” and 10 = “pain as bad as you can imagine”

Pain on Injection

In evaluating Pain on Injection, during each injection, the subject was asked to evaluate the pain of the injection (not the needle insertion). Pain on injection was rated using an 11 point Verbal Rank scale, where 0=no pain, and 10=pain as bad as you can imagine. Mean scores are summarized in Table B7. Pain on injection was rated highest by the 120K EDLA 0.625% treatment group (mean, 5.2) and lowest by the 120K EDLA 2.5% treatment group (mean, 2.6).

TABLE B7
Pain on Injection

| | Aqueous Bupivacaine | | 120K EDLA | | | |
|---|---|---|---|---|---|---|
| | 0.25% | 0.5% | 0.312% | 0.625% | 1.25% | 2.5% |
| Pain on Injection | N = 5 | N = 5 | N = 5 | N = 5 | N = 5 | N = 5 |
| Mean ± SE | 3.4 ± 1.2 | 3.8 ± 1.1 | 4.6 ± 1.4 | 5.2 ± 0.9 | 3.0 ± 0.8 | 2.6 ± 0.5 |

Summary of Efficacy

Sensory blockade data is summarized in Table B8. The incidence of analgesia/anesthesia among subjects who received 120K EDLA was not concentration dependent. Overall, 50% of subjects who received 120K EDLA experienced analgesia and none reported anesthesia. All subjects who received AB reported analgesia and 70% experienced anesthesia. The mean duration of analgesia/anesthesia ranged from 0 to 56 hours for the 120K EDLA treatment groups and from 16 to 39 hours for the AB treatment groups.

Thermal pain block occurred in 95% of 120K EDLA recipients, versus 90% of subjects who received AB. The mean onset of thermal pain block ranged from 1.6 hours to 50 hours post-injection for 120K EDLA and from 0.6 to 1.3 hours for AB. The mean duration of thermal pain block ranged from 89 to 234 hours for 120K EDLA and from 134 to 161 hours for AB.

Treatment with 120K EDLA resulted in altered thermal pain detection in 80% of subjects; AB treatment resulted in altered thermal pain detection for all subjects. The mean onset of altered thermal pain detection was less than 6 hours post-injection for 120K EDLA 0.312%, 0.625% and 1.25%, and 118 hours for 120K EDLA 2.5%. The mean onset of altered thermal pain detection ranged from 17 to 32 hours post-injection for AB. The duration of altered thermal pain detection ranged from 108 to 268 hours for 120K EDLA and from 163 to 206 hours for AB.

Heat block occurred in 15% of subjects receiving 120K EDLA, versus 60% of AB recipients. Mean onset of heat block was almost 10-fold less in subjects who received AB. The duration of heat block ranged from 0 to 108 hours for 120K EDLA and from 6.5 to 8 hours for AB.

Only 10% of 120K EDLA treatments resulted in tactile block while 70% percent of AB treatments resulted in tactile block. The mean onset of tactile block was 2.5 hours post-injection for 120K EDLA subjects and less than 0.5 hours for AB subjects. The duration of tactile block ranged from 0 to 3.5 hours for 120K EDLA and from 2 to 8 hours for AB.

All subjects who received 120K EDLA reported pain on suture placement but none of the subjects who received AB experienced pain on suture placement.

TABLE B8
Efficacy Summary-Incidence, Onset and Duration of Sensory Block

| | AB | | 120K EDLA | | | |
|---|---|---|---|---|---|---|
| | 0.25% | 0.5% | 0.312% | 0.625% | 1.25% | 2.5% |
| Sensory Block | N = 5 | N = 5 | N = 5 | N = 5 | N = 5 | N = 5 |
| Analgesia/Anesthesia | | | | | | |
| Number (%) of Subjects | 5(100) | 5(100) | 2(40) | 3(60) | 1(20) | 4(80) |
| Onset^{a} (hours) | 0.3 ± 0.1 | 0.3 ± 0.0 | 3.1 ± 2.9 | 1.2 ± 0.9 | 0.5 ± 0 | 24.7 ± 16.7 |
| mean ± SE | | | | | | |
| Duration^{b} (hours) | 16.3 ± 8.8 | 38.8 ± 26.5 | 0.0 ± 0.0 | 56.3 ± 55.7 | 0.5 ± 0 | 14.9 ± 11.4 |
| mean ± SE | | | | | | |
| Anesthesia | | | | | | |
| Number (%) of Subjects | 3(60) | 4(80) | 0 | 0 | 0 | 0 |
| Onset^{a} (hours) | 0.7 ± 0.2 | 0.9 ± 0.4 | 0 | 0 | 0 | 0 |
| mean ± SE | | | | | | |
| Duration^{b} (hours) | 4.8 ± 3.3 | 3.6 ± 2.8 | 0 | 0 | 0 | 0 |
| mean ± SE | | | | | | |
| Thermal Pain Block | | | | | | |
| Number (%) of Subjects | 4(80) | 5(100) | 5(100) | 5(100) | 4(80) | 5(100) |
| Onset^{a} (hours) | 1.3 ± 0.6 | 0.6 ± 0.2 | 19.2 ± 14.0 | 50.4 ± 32.3 | 20.5 ± 17.2 | 1.6 ± 0.7 |
| mean ± SE | | | | | | |
| Duration^{b} (hours) | 161.3 ± 68.0 | 133.8 ± 9.8 | 153.6 ± 49.0 | 88.8 ± 37.1 | 177.5 ± 52.9 | 233.6 ± 40.8 |
| mean ± SE | | | | | | |
| Altered Thermal Pain Detection | | | | | | |
| Number (%) of Subjects | 5(100) | 5(100) | 5(100) | 5(100) | 3(60) | 3(60) |
| Onset^{a} (hours) | 17.2 ± 8.8 | 31.8 ± 16.5 | 2.0 ± 1.0 | 5.4 ± 1.9 | 5.0 ± 3.5 | 118.0 ± 109.0 |
| mean ± SE | | | | | | |
| Duration^{b} (hours) | 162.8 ± 67.5 | 205.8 ± 57.0 | 209.2 ± 55.7 | 268.2 ± 61.6 | 143.0 ± 93.6 | 108.0 ± 108.0 |
| mean ± SE | | | | | | |
| Heat Block | | | | | | |
| Number (%) of Subjects | 2(40) | 4(80) | 0 | 1(20) | 0 | 2(40) |
| Onset^{a} (hours) | 1.0 ± 0.0 | 1.5 ± 0.3 | 0 | 12.0 | 0 | 18.0 ± 6.0 |
| mean ± SE | | | | | | |
| Duration^{b} (hours) | 8.0 ± 3.0 | 6.5 ± 2.7 | 0 | 108.0 | 0 | 0.0 ± 0.0 |
| mean ± SE | | | | | | |
| Tactile Block | | | | | | |
| Number (%) of Subjects | 4(80) | 3(60) | 0 | 1(20) | 0 | 1(20) |
| Onset^{a} (hours) | 0.4 ± 0.1 | 0.3 ± 0.0 | 0 | 2.5 | 0 | 2.5 |
| mean ± SE | | | | | | |
| Duration^{b} (hours) | 2.2 ± 1.3 | 7.8 ± 2.0 | 0 | 3.5 | 0 | 0.0 |
| mean ± SE | | | | | | |

N = number of subjects who received each treatment
^{a}Hours postinjection
^{b}Time from onset to offset

### CONCLUSION

Sensory blockade following subcutaneous infiltration of 120K EDLA was variable with respect to onset, duration and 120K EDLA concentration. One half of the subjects who received 120K EDLA reported analgesia but none reported anesthesia. All subjects who received subcutaneous infiltration of AB experienced analgesia and 70% experienced anesthesia. Most subjects exposed to 120K EDLA or AB experienced thermal pain block and altered thermal pain detection. Subjects who received 120K EDLA had a much lower incidence of heat block and of tactile block than did subjects who received AB.

Most adverse events were site-specific and were expected with this formulation. Most were mild and resolved without intervention. None of the adverse events was serious or severe. The relative absence of systemic adverse events suggested a safety profile characterized by minimal plasma bupivacaine concentrations.

### Example C

### The Sensory Blockade and Pharmacokinetics of EDLA and IDLA Administered as an Intercostal Nerve Block

A local anesthetic formulation prepared in accordance with Example 2 (EDLA) was administered to the intercostal nerves T9, T10, and T11. In Part 1 of the study, bilateral intercostal nerve blocks were administered using 40K EDLA and 120K EDLA in ascending doses to human subjects. Subjects received either 120K or 40K EDLA in one side and aqueous bupivacaine 0.25% in the other, thereby acting as their own controls in determining the effective dose of the test products. In Part 2, the two doses of 120K EDLA and 40K EDLA that demonstrated a 4-day duration of block in Part 1 were compared to equivalent doses of 120K and 40K IDLA (intermediate duration local anesthetic, incorporating bupivacaine in microspheres without dexamethasone, prepared in accordance with Example 1). Intercostal nerve blocks were administered to 1 side only (left side) using 40K EDLA and 120K EDLA, and an equivalent dose of 40K IDLA or 120K IDLA was administered in additional subjects for comparison. In Part 3, 5.0% 40K EDLA was administered in one side (left side). Plasma concentrations of bupivacaine and dexamethasone for each treatment group were determined.

All subjects were randomized as to which side received study drug vs. active comparator (0.25% aqueous bupivacaine). Bilateral segmental blocks to intercostal nerves T9, T10, and T11 were performed following standard practice used at Virginia Mason Clinic, as described in “Celiac and hypogastric plexus, intercostal, interpleural, and peripheral neural blockade of the thorax and abdomen,” by Kopacz, D. J. and Thompson, G. E., in Neural Blockade 3rd Edition, Cousins, M. J. and Bridenbaugh, P. O., Eds., New York, N.Y.: Lippincott-Raven Publishers, 1998, incorporated by reference herein. Skin infiltration over each block site was accomplished by making a skin wheal over the site using lidocaine 0.5-1% without epinephrine. The total dose of lidocaine used in this manner did not exceed 40 mg (4-8 mL). The blockade was made at the angle of the rib with the subject in the prone position. All EDLA or IDLA formulations were administered in volume of 2 mL per nerve (6 mL per side).

Tables C1 and C2 list the study treatments that were compared:

TABLE C1
Test and Reference Treatments

| | |
|---|---|
| Study Drug and Dose | Reference Treatment |

Part 1^{a}
Bilateral Injections (EDLA vs. Aq. Bupivacaine)

| | | |
|---|---|---|
| 120K EDLA | 40K EDLA | Aqueous Bupivacaine |
| — | 0.312% | 0.25% |
| 0.625% | 0.625% | 0.25% |
| 1.25% | 1.25% | 0.25% |
| 2.50% | 2.50% | 0.25% |

Part 2
Unilateral Injections

| | |
|---|---|
| 120K EDLA 1.25% | 120K IDLA 1.25% |
| 40K EDLA 2.50% | 40K IDLA 2.50% |

Part 3
Unilateral Injections

| | |
|---|---|
| 40K EDLA 5% | No comparator |

^{a}Subjects received bilateral injections of 120K EDLA or 40K EDLA to the intercostal nerves, T9, T10, and T11, on one side, and aqueous bupivacaine on the other side.

TABLE C2
Treatments Administered

| | Dose Form | Unit Strength (each mL) | |
|---|---|---|---|
| 120K EDLA 0.625%* | Suspension | Bupivacaine | 4.5 mg/mL |
| | | Dexamethasone | 2.5 μg/mL |
| 120K EDLA 1.25% | Suspension | Bupivacaine | 9.0 mg/mL |
| | | Dexamethasone | 5.0 μg/mL |
| 120K EDLA 2.5% | Suspension | Bupivacaine | 18.0 mg/mL |
| | | Dexamethasone | 10.0 μg/mL |
| 40K EDLA 0.312% | Suspension | Bupivacaine | 2.3 mg/mL |
| | | Dexamethasone | 1.2 μg/mL |
| 40K EDLA 0.625% | Suspension | Bupivacaine | 4.5 mg/mL |
| | | Dexamethasone | 2.5 μg/mL |
| 40K EDLA 1.25% | Suspension | Bupivacaine | 9.0 mg/mL |
| | | Dexamethasone | 5.0 μg/mL |
| 40K EDLA 2.5% | Suspension | Bupivacaine | 18.0 mg/mL |
| | | Dexamethasone | 10.0 μg/mL |
| 40K EDLA 5.0% | Suspension | Bupivacaine | 36.0 mg/mL |
| | | Dexamethasone | 20.0 μg/mL |
| 120K IDLA 1.25% | Suspension | Bupivacaine | 9.0 mg/mL |
| 40K IDLA 2.5% | Suspension | Bupivacaine | 18.0 mg/mL |
| AB 0.25% (Marcaine | Injectable | Bupivacaine | 2.5 mg/mL |
| HCI ®) | solution | | |

EDLA (120K and 40K) was supplied in 10 mL vials containing 100 mg of bupivacaine-loaded microspheres (approximately 72% by weight of bupivacaine and 0.04% dexamethasone).
IDLA (120K and 40K) was supplied in 10 mL vials containing 100 mg of bupivacaine-loaded microspheres (approximately 72% by weight of bupivacaine).

### Efficacy Testing

Sensory block was assessed in all subjects at 0, 15 minutes, 1, 2, 3, 6, 12, 24, 48, 72, and 96 hours post-injection and daily thereafter (approximately every 24 hours) until the block resolved using baseline pinprick, somesthetic testing (temperature perception block), and level of numbness tests, which were performed bilaterally. Subjects from all three parts of the study had blood samples taken for determination of plasma bupivacaine and dexamethasone levels at the same time points. The onset and duration of analgesia/anesthesia in response to pinprick (primary), incidence of anesthesia, onset and duration of temperature perception block, rate of unsuccessful blocks, degree of numbness, plasma drug concentrations over time, pharmacokinetic parameters (Cₘₐₓ, Tₘₐₓ, AUC), and degree of anesthesia/analgesia in relation to plasma drug concentrations were determined. Safety variables included pain on injection, local reaction at injection site, presence of other sensations/reactions (itching, tingling, burning, pain, hyperaesthesia), incidence and severity of adverse events, changes from baseline in vital signs and changes from baseline in laboratory tests (including hematology, clinical chemistry and urinalysis).

Pin-prick testing was performed as follows: The investigator assessed the degree of sensory block by administering pinpricks to the corresponding quadrant(s) of the abdomen at the mid-clavicular line in the area innervated by the intercostal nerves. Assessments were made by lightly tapping the skin on the quadrants of the abdomen using the dull end of a dental needle (or similar type needle). The density of sensory block was classified using the following criteria:

0=Subject did not feel any pinpricks.

1=Subject felt 2 or 3 (out of 3) pinpricks as TOUCH or PRESSURE.

2=Subject felt 2 or 3 (out of 3) pinpricks as SHARP.

If only 2 pinpricks were felt and 1 was felt as touch or pressure and the other was felt as sharp, or if only one pinprick was felt, the level of “1” (touch/pressure) was assigned.

Efficacy was also assessed in terms of onset, offset and duration of analgesia and/or anesthesia. Onset of analgesia was defined as the time at which pinprick testing demonstrated analgesia (touch/pressure) or anesthesia (no pinpricks felt) in a given area. Once the onset of sensory block was determined, the area(s) demonstrating the block were marked with a surgical pen. The areas outlined with the pen were designated as the pinprick test areas. All pinprick testing was subsequently contained within these site(s) in order to provide consistency of testing. Pinprick testing for onset of sensory block was performed by the investigator at pre-dose (baseline) and approximately 30 minutes, 1, 2, 3, 6 and 12 hours post-injection.

Duration of analgesia/anesthesia was defined as the time between onset of analgesia/anesthesia and time when there was a return of sensation of sharpness in response to pinprick. Offset of block was estimated as the midpoint in time between the last evaluation where analgesia/anesthesia was reported and the next evaluation where analgesia/anesthesia was no longer present. In the event of intermittent periods of analgesia, the total duration was the sum of these periods. Subjects returned to the study site approximately every 24 hours post-injection for pinprick testing by the investigator until the offset of sensory blockade was determined. Subjects were instructed on how to perform the pinprick method to self-evaluate the density of sensory block at home and record the results in a diary at least 1 time during the day, approximately every 12 hours following the investigator's assessment. Self-assessments continued for a total of 14 days, regardless of offset. Temperature perception block (somesthetic test) was assessed by touching the treated area with an alcohol swab. Subjects were instructed to answer “yes” if a change in temperature was felt, or “no” if no change was perceived. Onset of temperature perception block was defined as the first time at which the subject did not feel a change in temperature. Offset was defined as a return to baseline values for the somesthetic test.

Degree of numbness was measured as the distribution of numbness ratings at each time point, and was based on an 11-point numeric rating scale, where 0=not numb at all and 10=totally numb. Subjects were asked to rate the degree of numbness following touch to the sensory blocked areas on the abdomen.

Efficacy assessments using all testing modalities were performed by the investigator at baseline (hour 0), approximately 30 minutes, 1, 2, 3, 6 and 12 hours post-injection, and once every 24 hours until the block offset, and by the subject once every 24 hours (+/−90 minutes), approximately 12 hours following investigator assessments for 14 days regardless of offset of block. For the determination of plasma bupivacaine and dexamethasone concentrations, as well as standard pharmacokinetic measures (Cₘₐₓ, Tₘₐₓ, AUC), blood was drawn in Parts 1, 2, and 3 of the study at 0 hour (pre-dose), and at 15 minutes, 1, 2, 3, 6, 12, 24, 48, 72 and 96 hours post-injection and every 24 hours until the block offset.

Results

Analgesia and/or anesthesia, assessed by the response to pin-prick, is shown in Table C3 and FIGS. C1-C5, as a function of time after administration of EDLA or IDLA formulations or aqueous bupivacaine.

TABLE C3
Pin-Prick Results vs. Time
40K EDLA, 120K EDLA and IDLA

| | 40K | 40K | 40K | 40K | 120K | 120K | 120K | 120K | 40K | 40K | Aq. |
|---|---|---|---|---|---|---|---|---|---|---|---|
| | EDLA | EDLA | EDLA | EDLA | EDLA | EDLA | EDLA | IDLA | IDLA | EDLA | Bup. |
| | 0.312% | 0.625% | 1.25% | 2.50% | 0.625% | 1.25% | 2.50% | 1.25% | 2.5% | 5.0% | 0.25% |
| Baseline | | | | | | | | | | | |
| N | 3 | 6 | 6 | 3 | 3 | 3 | 3 | 1 | 6 | 6 | 18 |
| Mean | 2.00 | 2.00 | 2.00 | 2.00 | 2.00 | 2.00 | 2.00 | 2.00 | 2.00 | 2.00 | 2.00 |
| SE* | 0.00 | 0.00 | 0.00 | 0.00 | 0.00 | 0.00 | 0.00 | | 0.00 | 0.00 | 0.00 |
| Minimum | 2.00 | 2.00 | 2.00 | 2.00 | 2.00 | 2.00 | 2.00 | 2.00 | 2.00 | 2.00 | 2.00 |
| Median | 2.00 | 2.00 | 2.00 | 2.00 | 2.00 | 2.00 | 2.00 | 2.00 | 2.00 | 2.00 | 2.00 |
| Maximum | 2.00 | 2.00 | 2.00 | 2.00 | 2.00 | 2.00 | 2.00 | 2.00 | 2.00 | 2.00 | 2.00 |
| Hour 0.5 | | | | | | | | | | | |
| N | 3 | 6 | 6 | 3 | 3 | 3 | 3 | 1 | 6 | 6 | 18 |
| Mean | 2.00 | 2.00 | 1.83 | 2.00 | 2.00 | 2.00 | 2.00 | 2.00 | 1.67 | 1.67 | 0.44 |
| SE* | 0.00 | 0.00 | 0.17 | 0.00 | 0.00 | 0.00 | 0.00 | | 0.21 | 0.21 | 0.20 |
| Minimum | 2.00 | 2.00 | 1.00 | 2.00 | 2.00 | 2.00 | 2.00 | 2.00 | 1.00 | 1.00 | 0.00 |
| Median | 2.00 | 2.00 | 2.00 | 2.00 | 2.00 | 2.00 | 2.00 | 2.00 | 2.00 | 2.00 | 0.00 |
| Maximum | 2.00 | 2.00 | 2.00 | 2.00 | 2.00 | 2.00 | 2.00 | 2.00 | 2.00 | 2.00 | 2.00 |
| Hour 1 | | | | | | | | | | | |
| N | 3 | 6 | 6 | 3 | 3 | 3 | 3 | 1 | 6 | 6 | 18 |
| Mean | 2.00 | 1.83 | 1.50 | 1.33 | 2.00 | 2.00 | 2.00 | 2.00 | 1.67 | 1.00 | 0.11 |
| SE* | 0.00 | 0.17 | 0.34 | 0.67 | 0.00 | 0.00 | 0.00 | | 0.21 | 0.37 | 0.08 |
| Minimum | 2.00 | 1.00 | 0.00 | 0.00 | 2.00 | 2.00 | 2.00 | 2.00 | 1.00 | 0.00 | 0.00 |
| Median | 2.00 | 2.00 | 2.00 | 2.00 | 2.00 | 2.00 | 2.00 | 2.00 | 2.00 | 1.00 | 0.00 |
| Maximum | 2.00 | 2.00 | 2.00 | 2.00 | 2.00 | 2.00 | 2.00 | 2.00 | 2.00 | 2.00 | 1.00 |
| Hour 2 | | | | | | | | | | | |
| N | 3 | 6 | 6 | 3 | 3 | 3 | 3 | 1 | 6 | 6 | 18 |
| Mean | 2.00 | 1.50 | 1.17 | 0.33 | 2.00 | 2.00 | 2.00 | 2.00 | 1.50 | 0.83 | 0.06 |
| SE* | 0.00 | 0.22 | 0.40 | 0.33 | 0.00 | 0.00 | 0.00 | | 0.22 | 0.40 | 0.06 |
| Minimum | 2.00 | 1.00 | 0.00 | 0.00 | 2.00 | 2.00 | 2.00 | 2.00 | 1.00 | 0.00 | 0.00 |
| Median | 2.00 | 1.50 | 1.50 | 0.00 | 2.00 | 2.00 | 2.00 | 2.00 | 1.50 | 0.50 | 0.00 |
| Maximum | 2.00 | 2.00 | 2.00 | 1.00 | 2.00 | 2.00 | 2.00 | 2.00 | 2.00 | 2.00 | 1.00 |
| Hour 3 | | | | | | | | | | | |
| N | 3 | 6 | 6 | 3 | 3 | 3 | 3 | 1 | 6 | 6 | 18 |
| Mean | 2.00 | 1.00 | 0.83 | 0.00 | 2.00 | 2.00 | 2.00 | 2.00 | 1.17 | 0.50 | 0.06 |
| SE* | 0.00 | 0.26 | 0.40 | 0.00 | 0.00 | 0.00 | 0.00 | | 0.31 | 0.34 | 0.06 |
| Minimum | 2.00 | 0.00 | 0.00 | 0.00 | 2.00 | 2.00 | 2.00 | 2.00 | 0.00 | 0.00 | 0.00 |
| Median | 2.00 | 1.00 | 0.50 | 0.00 | 2.00 | 2.00 | 2.00 | 2.00 | 1.00 | 0.00 | 0.00 |
| Maximum | 2.00 | 2.00 | 2.00 | 0.00 | 2.00 | 2.00 | 2.00 | 2.00 | 2.00 | 2.00 | 1.00 |
| Hour 6 | | | | | | | | | | | |
| N | 3 | 6 | 6 | 3 | 3 | 3 | 3 | 1 | 6 | 6 | 18 |
| Mean | 1.00 | 0.50 | 0.00 | 0.00 | 2.00 | 1.00 | 2.00 | 2.00 | 0.67 | 0.33 | 0.56 |
| SE* | 0.58 | 0.34 | 0.00 | 0.00 | 0.00 | 0.58 | 0.00 | | 0.42 | 0.33 | 0.20 |
| Minimum | 0.00 | 0.00 | 0.00 | 0.00 | 2.00 | 0.00 | 2.00 | 2.00 | 0.00 | 0.00 | 0.00 |
| Median | 1.00 | 0.00 | 0.00 | 0.00 | 2.00 | 1.00 | 2.00 | 2.00 | 0.00 | 0.00 | 0.00 |
| Maximum | 2.00 | 2.00 | 0.00 | 0.00 | 2.00 | 2.00 | 2.00 | 2.00 | 2.00 | 2.00 | 2.00 |
| Hour 12 | | | | | | | | | | | |
| N | 3 | 6 | 6 | 3 | 3 | 3 | 3 | 1 | 6 | 6 | 18 |
| Mean | 1.33 | 0.50 | 0.00 | 0.00 | 2.00 | 1.00 | 1.67 | 2.00 | 1.00 | 0.17 | 2.00 |
| SE* | 0.67 | 0.34 | 0.00 | 0.00 | 0.00 | 0.58 | 0.33 | | 0.45 | 0.17 | 0.00 |
| Minimum | 2.00 | 0.00 | 0.00 | 0.00 | 2.00 | 0.00 | 1.00 | 2.00 | 0.00 | 0.00 | 2.00 |
| Median | 2.00 | 0.00 | 0.00 | 0.00 | 2.00 | 1.00 | 2.00 | 2.00 | 1.00 | 0.00 | 2.00 |
| Maximum | 2.00 | 2.00 | 0.00 | 0.00 | 2.00 | 2.00 | 2.00 | 2.00 | 2.00 | 1.00 | 2.00 |
| Day 1, | | | | | | | | | | | |
| morning | | | | | | | | | | | |
| N | 3 | 6 | 6 | 3 | 3 | 3 | 3 | 1 | 6 | 6 | 18 |
| Mean | 2.00 | 0.67 | 0.33 | 0.00 | 2.00 | 0.67 | 1.67 | 2.00 | 1.83 | 0.33 | 2.00 |
| SE* | 0.00 | 0.33 | 0.21 | 0.00 | 0.00 | 0.67 | 0.33 | | 0.17 | 0.21 | 0.00 |
| Minimum | 2.00 | 0.00 | 0.00 | 0.00 | 2.00 | 0.00 | 1.00 | 2.00 | 1.00 | 0.00 | 2.00 |
| Median | 2.00 | 0.50 | 0.00 | 0.00 | 2.00 | 0.00 | 2.00 | 2.00 | 2.00 | 0.00 | 2.00 |
| Maximum | 2.00 | 2.00 | 1.00 | 0.00 | 2.00 | 2.00 | 2.00 | 2.00 | 2.00 | 1.00 | 2.00 |
| Day 1, | | | | | | | | | | | |
| evening | | | | | | | | | | | |
| N | 3 | 6 | 6 | 3 | 3 | 3 | 3 | 1 | 6 | 6 | 18 |
| Mean | 2.00 | 1.33 | 1.33 | 0.33 | 2.00 | 1.33 | 2.00 | 1.00 | 1.83 | 0.50 | 2.00 |
| SE* | 0.00 | 0.33 | 0.33 | 0.33 | 0.00 | 0.33 | 0.00 | | 0.17 | 0.22 | 0.00 |
| Minimum | 2.00 | 0.00 | 2.00 | 0.00 | 2.00 | 1.00 | 2.00 | 1.00 | 1.00 | 0.00 | 2.00 |
| Median | 2.00 | 1.50 | 1.50 | 0.00 | 2.00 | 1.00 | 2.00 | 1.00 | 2.00 | 0.50 | 2.00 |
| Maximum | 2.00 | 2.00 | 2.00 | 1.00 | 2.00 | 2.00 | 2.00 | 1.00 | 2.00 | 1.00 | 2.00 |
| Day 2, | | | | | | | | | | | |
| morning | | | | | | | | | | | |
| N | 3 | 6 | 6 | 3 | 1 | 2 | 1 | 1 | 3 | 6 | 18 |
| Mean | 1.67 | 1.67 | 1.50 | 1.33 | 2.00 | 1.00 | 1.00 | 0.00 | 2.00 | 1.33 | 2.00 |
| SE* | 0.33 | 0.21 | 0.34 | 0.33 | | 0.00 | | | 0.00 | 0.33 | 0.00 |
| Minimum | 1.00 | 1.00 | 0.00 | 1.00 | 2.00 | 1.00 | 1.00 | 0.00 | 2.00 | 0.00 | 2.00 |
| Median | 2.00 | 2.00 | 2.00 | 1.00 | 2.00 | 1.00 | 1.00 | 0.00 | 2.00 | 1.50 | 2.00 |
| Maximum | 2.00 | 2.00 | 2.00 | 2.00 | 2.00 | 1.00 | 1.00 | 0.00 | 2.00 | 2.00 | 2.00 |
| Day 2, | | | | | | | | | | | |
| evening | | | | | | | | | | | |
| N | 3 | 6 | 6 | 3 | 3 | 3 | 3 | 1 | 6 | 6 | 18 |
| Mean | 2.00 | 1.83 | 1.50 | 2.00 | 2.00 | 1.67 | 1.67 | 2.00 | 2.00 | 1.17 | 1.94 |
| SE* | 0.00 | 0.17 | 0.34 | 0.00 | 0.00 | 0.33 | 0.33 | | 0.00 | 0.17 | 0.06 |
| Minimum | 2.00 | 1.00 | 0.00 | 2.00 | 2.00 | 1.00 | 1.00 | 2.00 | 2.00 | 1.00 | 1.00 |
| Median | 2.00 | 2.00 | 2.00 | 2.00 | 2.00 | 2.00 | 2.00 | 2.00 | 2.00 | 1.00 | 2.00 |
| Maximum | 2.00 | 2.00 | 2.00 | 2.00 | 2.00 | 2.00 | 2.00 | 2.00 | 2.00 | 2.00 | 2.00 |
| Day 3, | | | | | | | | | | | |
| morning | | | | | | | | | | | |
| N | 3 | 3 | 2 | 2 | — | 2 | 1 | 1 | 2 | 6 | 10 |
| Mean | 2.00 | 2.00 | 1.50 | 2.00 | — | 1.00 | 1.00 | 0.00 | 2.00 | 1.50 | 2.00 |
| SE* | 0.00 | 0.00 | 0.50 | 0.00 | — | 0.00 | | | 0.00 | 0.22 | 0.00 |
| Minimum | 2.00 | 2.00 | 1.00 | 2.00 | — | 1.00 | 1.00 | 0.00 | 2.00 | 1.00 | 2.00 |
| Median | 2.00 | 2.00 | 1.50 | 2.00 | — | 1.00 | 2.00 | 0.00 | 2.00 | 1.50 | 2.00 |
| Maximum | 2.00 | 2.00 | 2.00 | 2.00 | — | 1.00 | 2.00 | 0.00 | 2.00 | 2.00 | 2.00 |
| Day 3, | | | | | | | | | | | |
| evening | | | | | | | | | | | |
| N | 3 | 6 | 6 | 3 | 3 | 3 | 3 | 1 | 6 | 6 | 18 |
| Mean | 2.00 | 2.00 | 1.83 | 2.00 | 2.00 | 1.67 | 1.67 | 1.00 | 2.00 | 1.17 | 2.00 |
| SE* | 0.00 | 0.00 | 0.17 | 0.00 | 0.00 | 0.33 | 0.33 | | 0.00 | 0.31 | 0.00 |
| Minimum | 2.00 | 2.00 | 1.00 | 2.00 | 2.00 | 1.00 | 1.00 | 1.00 | 2.00 | 0.00 | 2.00 |
| Median | 2.00 | 2.00 | 2.00 | 2.00 | 2.00 | 2.00 | 2.00 | 1.00 | 2.00 | 1.00 | 2.00 |
| Maximum | 2.00 | 2.00 | 2.00 | 2.00 | 2.00 | 2.00 | 2.00 | 1.00 | 2.00 | 2.00 | 2.00 |
| Day 4, | | | | | | | | | | | |
| morning | | | | | | | | | | | |
| N | 3 | — | — | 1 | — | 2 | 1 | 1 | 2 | 4 | 3 |
| Mean | 2.00 | — | — | 2.00 | — | 2.00 | 2.00 | 1.00 | 2.00 | 1.25 | 2.00 |
| SE* | 0.00 | — | — | | — | 0.00 | | | 0.00 | 0.25 | 0.00 |
| Minimum | 2.00 | — | — | 2.00 | — | 2.00 | 2.00 | 1.00 | 2.00 | 1.00 | 2.00 |
| Median | 2.00 | — | — | 2.00 | — | 2.00 | 2.00 | 1.00 | 2.00 | 1.00 | 2.00 |
| Maximum | 2.00 | — | — | 2.00 | — | 2.00 | 2.00 | 1.00 | 2.00 | 2.00 | 2.00 |
| Day 4, | | | | | | | | | | | |
| evening | | | | | | | | | | | |
| N | 3 | 6 | 6 | 3 | 3 | 3 | 3 | 1 | 6 | 6 | 18 |
| Mean | 2.00 | 2.00 | 2.00 | 2.00 | 2.00 | 2.00 | 2.00 | 1.00 | 2.00 | 1.50 | 2.00 |
| SE* | 0.00 | 0.00 | 0.00 | 0.00 | 0.00 | 0.00 | 0.00 | | 0.00 | 0.22 | 0.00 |
| Minimum | 2.00 | 2.00 | 2.00 | 2.00 | 2.00 | 2.00 | 2.00 | 1.00 | 2.00 | 1.00 | 2.00 |
| Median | 2.00 | 2.00 | 2.00 | 2.00 | 2.00 | 2.00 | 2.00 | 1.00 | 2.00 | 1.50 | 2.00 |
| Maximum | 2.00 | 2.00 | 2.00 | 2.00 | 2.00 | 2.00 | 2.00 | 1.00 | 2.00 | 2.00 | 2.00 |
| Day 5, | | | | | | | | | | | |
| morning | | | | | | | | | | | |
| N | | — | — | — | — | — | — | — | — | 3 | 18 |
| Mean | | — | — | — | — | — | — | — | — | 1.67 | 2.00 |
| SE* | — | — | — | — | — | — | — | — | — | 0.33 | 0.00 |
| Minimum | — | — | — | — | — | — | — | — | — | 1.00 | 2.00 |
| Median | — | — | — | — | — | — | — | — | — | 2.00 | 2.00 |
| Maximum | — | — | — | — | — | — | — | — | — | 2.00 | 2.00 |
| Day 5, | | | | | | | | | | | |
| evening | | | | | | | | | | | |
| N | — | — | — | — | — | — | — | 1 | 5 | 6 | 18 |
| Mean | — | — | — | — | — | — | — | 2.00 | 2.00 | 1.83 | 2.00 |
| SE* | — | — | — | — | — | — | — | | 0.00 | 0.17 | 0.00 |
| Minimum | — | — | — | — | — | — | — | 2.00 | 2.00 | 1.00 | 2.00 |
| Median | — | — | — | — | — | — | — | 2.00 | 2.00 | 2.00 | 2.00 |
| Maximum | — | — | — | — | — | — | — | 2.00 | 2.00 | 2.00 | 2.00 |
| Day 6, | | | | | | | | | | | |
| morning | | | | | | | | | | | |
| N | — | — | — | — | — | — | — | — | — | 1 | 18 |
| Mean | — | — | — | — | — | — | — | — | — | 1.00 | 2.00 |
| SE* | — | — | — | — | — | — | — | — | — | | 0.00 |
| Minimum | — | — | — | — | — | — | — | — | — | 1.00 | 2.00 |
| Median | — | — | — | — | — | — | — | — | — | 1.00 | 2.00 |
| Maximum | — | — | — | — | — | — | — | — | — | 1.00 | 2.00 |
| Day 6, | | | | | | | | | | | |
| evening | | | | | | | | | | | |
| N | — | — | — | — | — | — | — | 1 | 5 | 6 | 18 |
| Mean | — | — | — | — | — | — | — | 2.00 | 2.00 | 1.83 | 2.00 |
| SE* | — | — | — | — | — | — | — | | 0.00 | 0.17 | 0.00 |
| Minimum | — | — | — | — | — | — | — | 2.00 | 2.00 | 1.00 | 2.00 |
| Median | — | — | — | — | — | — | — | 2.00 | 2.00 | 2.00 | 2.00 |
| Maximum | — | — | — | — | — | — | — | 2.00 | 2.00 | 2.00 | 2.00 |
| Day 7, | | | | | | | | | | | |
| morning | | | | | | | | | | | |
| N | — | — | — | — | — | — | — | — | — | 1 | 18 |
| Mean | — | — | — | — | — | — | — | — | — | 2.00 | 2.00 |
| SE* | — | — | — | — | — | — | — | — | — | | 0.00 |
| Minimum | — | — | — | — | — | — | — | — | — | 2.00 | 2.00 |
| Median | — | — | — | — | — | — | — | — | — | 2.00 | 2.00 |
| Maximum | — | — | — | — | — | — | — | — | — | 2.00 | 2.00 |
| Day 7, | | | | | | | | | | | |
| evening | | | | | | | | | | | |
| N | — | — | — | — | — | — | — | 1 | 6 | 6 | 18 |
| Mean | — | — | — | — | — | — | — | 2.00 | 2.00 | 2.00 | 2.00 |
| SE* | — | — | — | — | — | — | — | | 0.00 | 0.00 | 0.00 |
| Minimum | — | — | — | — | — | — | — | 2.00 | 2.00 | 2.00 | 2.00 |
| Median | — | — | — | — | — | — | — | 2.00 | 2.00 | 2.00 | 2.00 |
| Maximum | — | — | — | — | — | — | — | 2.00 | 2.00 | 2.00 | 2.00 |

Baseline = Day 1, pre-injection.
*SE = Standard Error.
Level of Pin-Prick: 0 = no feeling; 1 = touch/pressure; 2 = sharp.

As can be seen from the results set forth in Table C3 and FIGS. C1-C5, the sensation of a pin-prick as touch or pressure or no sensation of pin-prick was achieved within 6 hours of administration of EDLA or IDLA formulations, and in some instances, within 1 to 3 hours. The time to return to normal sensation varied between 1 to 4 days and up to at least 10 days with some formulations. Time to maximum effect varied as well, with maximum effect being achieved between 6 hours and 2 days and up to 9 days after administration of certain formulations. In contrast, analgesia and/or anesthesia from 0.25% aqueous bupivacaine was achieved within 0.5 to 1 hour but had completely disappeared by 12 hours after administration.

Onset of analgesia was defined as the time at which pinprick testing demonstrated analgesia (touch/pressure) or anesthesia (no pinpricks felt) in a given area. Duration of analgesia/anesthesia was defined as the time between onset of analgesia/anesthesia and time when there was a return of sensation of sharpness in response to pinprick. Offset of block was estimated as the midpoint in time between the last evaluation where analgesia/anesthesia was reported and the next evaluation where analgesia/anesthesia was no longer present.

As reported in Table C4 and depicted graphically in FIGS. C1, C2 and C5, onset of block (as defined above) occurred within 3-6 hours in 89% of subjects across all 40K EDLA doses, compared to 22% of subjects in the 120K EDLA groups. Onset of block was observed within 1 to 3 hours in 80% of 40K EDLA blocks. Aqueous bupivacaine 0.25% had the shortest onset of analgesia/anesthesia, with 100% of subjects experiencing onset within 1 hour. The 2.5% dose/concentration of 40K EDLA had the most rapid onset, with 100% of subjects reporting analgesia/anesthesia onset within 2 hours (vs. 0.312%=0%, 0.625%=50%, 1.25%=50%). The maximum dose of 40K EDLA administered in this study, 5.0%, had an onset similar to that of the other 40K groups, with 66% of subjects having analgesia/anesthesia onset within 1 hour. Among subjects receiving 120K EDLA, the 1.25% dose had the most rapid onset, with 67% of subjects reporting analgesia/anesthesia within 3-6 hours.

The 2.5% concentration of 40K EDLA was selected in Part 1 for comparison with the equivalent dose 40K IDLA in Part 2. Results showed that 40K EDLA 2.5% had a slightly more rapid onset of block compared to 40K IDLA 2.5%, with 66% of successful sensory blocks occurring within 2 hours, vs 50% for IDLA. These results suggest that the 40K formulation (both EDLA and IDLA) produces a more rapid onset of analgesia/anesthesia than the 120K formulation, and that EDLA has a slightly more rapid onset than IDLA. Table C4 summarizes these results.

FIG. C3 illustrates the overall time course of analgesia for 40K EDLA 2.5% and 40K IDLA 2.5%, and shows that the onset of block was similar for both 40K EDLA 2.5% and 40K IDLA, while duration for 40K EDLA was longer (2 days vs 1 day, respectively). The overall time course of analgesia for 120K IDLA 1.25% was longer in comparison with the 40K formulations in terms of both the onset and the duration of the block, with a peak effect at 48 hours, and return to normal sensation by 5 days (see FIG. C4). The overall time course of analgesia for 40K EDLA 5.0%, as shown in FIG. C5, shows an onset of block that is comparable to the 40K EDLA and IDLA 2.5% formulations, and demonstrates a duration of block that is longer in comparison with treatment with 40K formulations at a lower concentration of local anesthetic.

TABLE C4
Time to Onset of Analgesia/Anesthesia^{a} (Number (%))
Study Part 1: EDLA 120K

| | EDLA | EDLA | EDLA | AB^{b} |
|---|---|---|---|---|
| | 0.625% | 1.25% | 2.5% | 0.25% |
| Time to Onset | (N = 3) | (N = 3) | (N = 3) | (N = 9) |
| ≦30 min. | 0 | 0 | 0 | 9 (100%) |
| >30 min. < 1 h | 0 | 0 | 0 | 0 |
| >1-2 h | 0 | 0 | 0 | 0 |
| >2-3 h | 0 | 0 | 0 | 0 |
| >3-6 h | 0 | 2 (67%) | 0 | 0 |
| >6-12 h | 0 | 0 | 1 (33%) | 0 |
| >12 h | 0 | 1 (33%) | 1 (33%) | 0 |

Study Part 1: EDLA 40K

| | EDLA | EDLA | EDLA | EDLA | AB^{b} |
|---|---|---|---|---|---|
| | 0.312% | 0.625% | 1.25% | 2.5% | 0.25% |
| | (N = 3) | (N = 6) | (N = 6) | (N = 3) | (N = 18) |
| ≦30 min. | 0 | 0 | 1 (17%) | 0 | 14 (78%) |
| >30 min. < 1 h | 0 | 1 (17%) | 1 (17%) | 1 (33%) | 4 (22%) |
| >1-2 h | 0 | 2 (33%) | 1 (17%) | 2 (67%) | 0 |
| >2-3 h | 0 | 2 (33%) | 1 (17%) | 0 | 0 |
| >3-6 h | 2 (67%) | 0 | 2 (33%) | 0 | 0 |
| >6-12 h | 0 | 0 | 0 | 0 | 0 |
| >12 h | 1 (33%) | 0 | 0 | 0 | 0 |

| Study Part 2 | | | | | Study Part 3 |
|---|---|---|---|---|---|
| | EDLA | IDLA | EDLA | IDLA | 40K |
| | 120K | 120K | 40K | 40K | EDLA |
| | 1.25% | 1.25% | 2.5% | 2.5% | 5.0% |
| Time to Onset | (N = 2) | (N = 1) | (N = 6) | (N = 6) | (N = 6) |
| ≦30 min. | 0 | 0 | 2 (33%) | 2 (33%) | 2 (33%) |
| >30 min. < 1 h | 0 | 0 | 2 (33%) | 0 | 2 (33%) |
| >1-2 h | 0 | 0 | 0 | 1 (17%) | 0 |
| >2-3 h | 0 | 0 | 1 (17%) | 1 (17%) | |
| >3-6 h | 0 | 0 | 1 (17%) | 0 | 0 |
| >6-12 h | 0 | 0 | 0 | 0 | 1 (17%) |
| >12-h | 0 | 1 (100%) | 0 | 0 | 0 |

Note:
Columns resulting in fewer than 100% of subjects represented are due to unsuccessful sensory blocks (subjects did not experience analgesia/anesthesia).
^{a}Analgesia = subjects felt 2 or 3 of 3 pinpricks as touch/pressure. Anesthesia = subjects did not feel any of 3 pinpricks
^{b}AB data are averaged over treatment group.

Duration of analgesia/anesthesia was defined as the time between onset of analgesia/anesthesia and the time when there was a return of a sensation of sharpness to pinprick testing (i.e., loss of analgesia/anesthesia). Duration of analgesia was longer for 120K EDLA 2.5% compared to 40K EDLA 2.5% (75.0 hours vs 44.3 hours), and both formulations had longer duration than aqueous bupivacaine (from 7 to 10 hours). Table C5 summarizes the results.

A dose-response relationship was apparent for both the 120K EDLA (1.25%=64 hours, 2.5%=75 hours) and 40K EDLA (0.312%=5 hours, 0.625%=39 hours, 1.25%=hours; 2.5%=44 hours). The 0.312% concentration of 40K EDLA showed slight efficacy, with a duration of block that was shorter than that for AB (5 vs 8 hours). The maximum concentration of 40K EDLA (5.0%) almost doubled the duration of block compared to the 2.5% concentration (86 hours compared to 44.5 hours, respectively). The 120K IDLA formulation at 1.25% produced a relatively long duration of analgesia/anesthesia (72 hours).

The 2.5% concentration of 40K EDLA was selected in Part 1 for comparison with 40K IDLA in Part 2. As FIG. C3 shows, duration of analgesia/anesthesia was longer for 2.5% 40K EDLA (45 hours) compared to 2.5% 40K IDLA (20 hours). These data support previous findings showing that dexamethasone prolongs the duration of action of bupivacaine.

TABLE C5
Mean Duration (hours) of Analgesia/Anesthesia
Study Part 1

| | | | |
|---|---|---|---|
| EDLA 120K | EDLA 120K | EDLA 120K | AB^{b} |
| 0.625% | 1.25% | 2.5% | 0.25% |
| (N = 3) | (N = 3) | (N = 3) | (N = 9) |

Duration (hours) Mean (SE)

| | | | |
|---|---|---|---|
| 0 | 64.0 (11.1) | 75.0 (9) | 10.3 (2.8) |

Study Part 1

| | | | | |
|---|---|---|---|---|
| EDLA 40K | EDLA 40K | EDLA 40K | EDLA 40K | AB^{b} |
| 0.312% | 0.625% | 1.25% | 2.5% | 0.25% |
| (N = 3) | (N = 6) | (N = 6) | (N = 3) | (N = 18) |

Duration (hours) Mean (SE)

| 5.0 (3.6) | 38.6 (5.6) | 42.9 (9.8) | 44.3 (2.2) | 8.1 (0.9) |
|---|---|---|---|---|
| Study Part 2 | | | | Study Part 3 |
| EDLA 120K | IDLA 120K | EDLA 40K | IDLA 40K | 40K EDLA |
| 1.25% | 1.25% | 2.5% | 2.5% | 5.0% |
| (N = 2) | (N = 1) | (N = 6) | (N = 6) | (N = 6) |

Duration (hours) Mean (SE)

| | | | | |
|---|---|---|---|---|
| 0 | 72.0 (0) | 44.5 (10.1) | 20.3 (7.4) | 86.0 (17.0) |

^{a}Not all subjects in all dose/treatment groups experienced successful sensory blocks.
^{b}AB data are averaged over treatment group

### Incidence of Analgesia/Anesthesia

Across all 40K EDLA dose groups, analgesia was observed in 67% to 100% of subjects. A dose-response effect was evident with respect to percent of subjects experiencing analgesia: 0.312%=67%, 0.625%=83%, 1.25%=67%, 2.5%=100%; and 5.0%=100 of subjects, and anesthesia: 0.312%=33%, 0.625%=67%, 1.25%=100%, 2.5%=100%; and 5.0%=83% of subjects. For comparison, in the subjects tested with aqueous bupivacaine, analgesia was observed in 100% of subjects, and anesthesia was observed in 83% of subjects. In contrast, in the 120K EDLA groups, 0-100% of the subjects reported analgesia; and 0-67% of the subjects experienced anesthesia.

In the 40K 2.5% comparison, EDLA was more closely associated with analgesia than was IDLA (EDLA=100% of subjects; IDLA=67% of subjects). The 5.0% concentration of 40K EDLA resulted in anesthesia in 83% of subjects and analgesia in 100% of subjects. Results are summarized by treatment group in Table C6.

TABLE C6
Incidence of Analgesia/Anesthesia (Number (%))
Study Part 1

| | | | |
|---|---|---|---|
| EDLA 120K | EDLA 120K | EDLA 120K | AB^{a} |
| 0.625% | 1.25% | 2.5% | 0.25% |
| (N = 3) | (N = 3) | (N = 3) | (N = 9) |

No. (%) with analgesia^{b}

| | | | |
|---|---|---|---|
| 0 | 3 (100%) | 2 (67%) | 9 (100%) |

No. (%) with anesthesia^{c}

| | | | |
|---|---|---|---|
| 0 | 2 (67%) | 2 (67%) | 9 (100%) |

Study Part 1

| | | | | |
|---|---|---|---|---|
| EDLA 40K | EDLA 40K | EDLA 40K | EDLA 40K | AB^{a} |
| 0.312% | 0.625% | 1.25% | 2.5% | 0.25% |
| (N = 3) | (N = 6) | (N = 6) | (N = 3) | (N = 18) |

No. (%) with analgesia^{b}

| | | | | |
|---|---|---|---|---|
| 2 (67%) | 5 (83%) | 4 (67%) | 3 (100%) | 18 (100%) |

No. (%) with anesthesia^{c}

| | | | | |
|---|---|---|---|---|
| 1 (33%) | 4 (67%) | 6 (100%) | 3 (100%) | 18 (100%) |

| Study Part 2 | | | | Study Part 3 |
|---|---|---|---|---|
| EDLA 120K | IDLA 120K | EDLA 40K | IDLA 40K | EDLA 40K |
| 1.25% | 1.25% | 2.5% | 2.5% | 5.0% |
| (N = 2) | (N = 1) | (N = 6) | (N = 6) | (N = 6) |

No. (%) with analgesia^{b}

| | | | | |
|---|---|---|---|---|
| 0 | 1 (100%) | 6 (100%) | 4 (67%) | 6 (100%) |

No. (%) with anesthesia^{c}

| | | | | |
|---|---|---|---|---|
| 0 | 1 (100%) | 3 (50%) | 4 (67%) | 5 (83%) |

^{a}AB data were averaged over treatment groups
^{b}Subjects felt 2 or 3 of the 3 pinpricks as touch/pressure
^{c}Subjects did not feel any of 3 pinpricks

Somesthetic Testing

Analgesia and anesthesia, assessed by the response to somesthetic testing (temperature perception), is shown in Table C7 below, as a function of time after administration of EDLA or IDLA formulations or aqueous bupivacaine.

TABLE C7
Somesthetic Test Results vs. Time
40K EDLA, 120K EDLA and IDLA

| | 40K | 40K | 40K | 40K | 120K | 120K | 120K | 120K | 40K | 40K | Aq. |
|---|---|---|---|---|---|---|---|---|---|---|---|
| | EDLA | EDLA | EDLA | EDLA | EDLA | EDLA | EDLA | IDLA | IDLA | EDLA | Bup. |
| | 0.312% | 0.625% | 1.25% | 2.5% | 0.625% | 1.25% | 2.50% | 1.25% | 2.5% | 5.0% | 0.25% |
| Baseline | | | | | | | | | | | |
| N | 3 | 6 | 6 | 3 | 3 | 3 | 3 | 1 | 6 | 6 | 6 |
| Mean | 1.00 | 1.00 | 1.00 | 1.00 | 1.00 | 1.00 | 1.00 | 1.00 | 1.00 | 1.00 | 1.00 |
| SE* | 0.00 | 0.00 | 0.00 | 0.00 | 0.00 | 0.00 | 0.00 | | 0.00 | 0.00 | 0.00 |
| Minimum | 1.00 | 1.00 | 1.00 | 1.00 | 1.00 | 1.00 | 1.00 | 1.00 | 1.00 | 1.00 | 1.00 |
| Median | 1.00 | 1.00 | 1.00 | 1.00 | 1.00 | 1.00 | 1.00 | 1.00 | 1.00 | 1.00 | 1.00 |
| Maximum | 1.00 | 1.00 | 1.00 | 1.00 | 1.00 | 1.00 | 1.00 | 1.00 | 1.00 | 1.00 | 1.00 |
| Hour 0.5 | | | | | | | | | | | |
| N | 3 | 6 | 6 | 3 | 3 | 3 | 3 | 1 | 6 | 6 | 6 |
| Mean | 1.00 | 1.00 | 1.00 | 1.00 | 1.00 | 1.00 | 1.00 | 1.00 | 1.00 | 0.83 | 0.00 |
| SE* | 0.00 | 0.00 | 0.00 | 0.00 | 0.00 | 0.00 | 0.00 | | 0.00 | 0.17 | 0.00 |
| Minimum | 1.00 | 1.00 | 1.00 | 1.00 | 1.00 | 1.00 | 1.00 | 1.00 | 1.00 | 0.00 | 0.00 |
| Median | 1.00 | 1.00 | 1.00 | 1.00 | 1.00 | 1.00 | 1.00 | 1.00 | 1.00 | 1.00 | 0.00 |
| Maximum | 1.00 | 1.00 | 1.00 | 1.00 | 1.00 | 1.00 | 1.00 | 1.00 | 1.00 | 1.00 | 0.00 |
| Hour 1 | | | | | | | | | | | |
| N | 3 | 6 | 6 | 3 | 3 | 3 | 3 | 1 | 6 | 6 | 6 |
| Mean | 1.00 | 0.83 | 0.83 | 1.00 | 1.00 | 1.00 | 1.00 | 1.00 | 0.83 | 0.83 | 0.00 |
| SE* | 0.00 | 0.17 | 0.17 | 0.00 | 0.00 | 0.00 | 0.00 | | 0.17 | 0.17 | 0.00 |
| Minimum | 1.00 | 0.00 | 0.00 | 1.00 | 1.00 | 1.00 | 1.00 | 1.00 | 0.00 | 0.00 | 0.00 |
| Median | 1.00 | 1.00 | 1.00 | 1.00 | 1.00 | 1.00 | 1.00 | 1.00 | 1.00 | 1.00 | 0.00 |
| Maximum | 1.00 | 1.00 | 1.00 | 1.00 | 1.00 | 1.00 | 1.00 | 1.00 | 1.00 | 1.00 | 0.00 |
| Hour 2 | | | | | | | | | | | |
| N | 3 | 6 | 6 | 3 | 3 | 3 | 3 | 1 | 6 | 6 | 6 |
| Mean | 1.00 | 1.00 | 0.67 | 0.00 | 1.00 | 1.00 | 1.00 | 1.00 | 0.83 | 0.67 | 0.00 |
| SE* | 0.00 | 0.00 | 0.21 | 0.00 | 0.00 | 0.00 | 0.00 | | 0.17 | 0.21 | 0.00 |
| Minimum | 1.00 | 1.00 | 0.00 | 0.00 | 1.00 | 1.00 | 1.00 | 1.00 | 0.00 | 0.00 | 0.00 |
| Median | 1.00 | 1.00 | 1.00 | 0.00 | 1.00 | 1.00 | 1.00 | 1.00 | 1.00 | 1.00 | 0.00 |
| Maximum | 1.00 | 1.00 | 1.00 | 0.00 | 1.00 | 1.00 | 1.00 | 1.00 | 1.00 | 1.00 | 0.00 |
| Hour 3 | | | | | | | | | | | |
| N | 3 | 6 | 6 | 3 | 3 | 3 | 3 | 1 | 6 | 6 | 6 |
| Mean | 1.00 | 0.83 | 0.33 | 0.00 | 1.00 | 1.00 | 1.00 | 1.00 | 0.50 | 0.83 | 0.17 |
| SE* | 0.00 | 0.17 | 0.21 | 0.00 | 0.00 | 0.00 | 0.00 | | 0.22 | 0.17 | 0.17 |
| Minimum | 1.00 | 0.00 | 0.00 | 0.00 | 1.00 | 1.00 | 1.00 | 1.00 | 0.00 | 0.00 | 0.00 |
| Median | 1.00 | 1.00 | 0.00 | 0.00 | 1.00 | 1.00 | 1.00 | 1.00 | 0.50 | 1.00 | 0.00 |
| Maximum | 1.00 | 1.00 | 1.00 | 0.00 | 1.00 | 1.00 | 1.00 | 1.00 | 1.00 | 1.00 | 1.00 |
| Hour 6 | | | | | | | | | | | |
| N | 3 | 6 | 6 | 3 | 3 | 3 | 3 | 1 | 6 | 6 | 6 |
| Mean | 0.67 | 0.33 | 0.00 | 0.00 | 1.00 | 0.67 | 1.00 | 1.00 | 0.50 | 0.67 | 0.33 |
| SE* | 0.33 | 0.21 | 0.00 | 0.00 | 0.00 | 0.33 | 0.00 | | 0.22 | 0.21 | 0.21 |
| Minimum | 0.00 | 0.00 | 0.00 | 0.00 | 1.00 | 0.00 | 1.00 | 1.00 | 0.00 | 0.00 | 0.00 |
| Median | 1.00 | 0.00 | 0.00 | 0.00 | 1.00 | 1.00 | 1.00 | 1.00 | 0.50 | 1.00 | 0.00 |
| Maximum | 1.00 | 1.00 | 0.00 | 0.00 | 1.00 | 1.00 | 1.00 | 1.00 | 1.00 | 1.00 | 1.00 |
| Hour 12 | | | | | | | | | | | |
| N | | | | | | | | | | | |
| Mean | 3 | 6 | 6 | 3 | 3 | 3 | 3 | 1 | 6 | 6 | 6 |
| SE* | 0.67 | 0.33 | 0.00 | 0.00 | 1.00 | 0.33 | 0.67 | 1.00 | 0.67 | 0.00 | 0.83 |
| Minimum | 0.33 | 0.21 | 0.00 | 0.00 | 0.00 | 0.33 | 0.33 | | 0.21 | 0.00 | 0.17 |
| Median | 0.00 | 0.00 | 0.00 | 0.00 | 1.00 | 0.00 | 0.00 | 1.00 | 0.00 | 0.00 | 0.00 |
| Maximum | 1.00 | 0.00 | 0.00 | 0.00 | 1.00 | 0.00 | 1.00 | 1.00 | 1.00 | 0.00 | 1.00 |
| | 1.00 | 1.00 | 0.00 | 0.00 | 1.00 | 1.00 | 1.00 | 1.00 | 1.00 | 0.00 | 1.00 |
| Day 1, | | | | | | | | | | | |
| morning | | | | | | | | | | | |
| N | 3 | 6 | 6 | 3 | 3 | 3 | 3 | 1 | 6 | 6 | 6 |
| Mean | 1.00 | 0.33 | 0.33 | 0.00 | 1.00 | 0.33 | 0.67 | 1.00 | 0.83 | 0.00 | 1.00 |
| SE* | 0.00 | 0.21 | 0.21 | 0.00 | 0.00 | 0.33 | 0.33 | | 0.17 | 0.00 | 0.00 |
| Minimum | 1.00 | 0.00 | 0.00 | 0.00 | 1.00 | 0.00 | 0.00 | 1.00 | 0.00 | 0.00 | 1.00 |
| Median | 1.00 | 0.00 | 0.00 | 0.00 | 1.00 | 0.00 | 1.00 | 1.00 | 1.00 | 0.00 | 1.00 |
| Maximum | 1.00 | 1.00 | 1.00 | 0.00 | 1.00 | 1.00 | 1.00 | 1.00 | 1.00 | 0.00 | 1.00 |
| Day 1, | | | | | | | | | | | |
| evening | | | | | | | | | | | |
| N | 3 | 6 | 6 | 3 | 3 | 3 | 3 | 1 | 6 | 6 | 6 |
| Mean | 1.00 | 0.67 | 0.83 | 0.00 | 1.00 | 0.33 | 0.67 | 1.00 | 0.83 | 0.00 | 1.00 |
| SE* | 0.00 | 0.21 | 0.17 | 0.00 | 0.00 | 0.33 | 0.33 | | 0.17 | 0.00 | 0.00 |
| Minimum | 1.00 | 0.00 | 0.00 | 0.00 | 1.00 | 0.00 | 0.00 | 1.00 | 0.00 | 0.00 | 1.00 |
| Median | 1.00 | 1.00 | 1.00 | 0.00 | 1.00 | 0.00 | 1.00 | 1.00 | 1.00 | 0.00 | 1.00 |
| Maximum | 1.00 | 1.00 | 1.00 | 0.00 | 1.00 | 1.00 | 1.00 | 1.00 | 1.00 | 0.00 | 1.00 |
| Day 2, | | | | | | | | | | | |
| morning | | | | | | | | | | | |
| N | 3 | 6 | 6 | 3 | 1 | 2 | 1 | 1 | 3 | 6 | 6 |
| Mean | 0.67 | 1.00 | 1.00 | 1.00 | 1.00 | 0.00 | 0.00 | 0.00 | 1.00 | 0.17 | 1.00 |
| SE* | 0.33 | 0.00 | 0.00 | 0.00 | | 0.00 | | | 0.00 | 0.17 | 0.00 |
| Minimum | 0.00 | 1.00 | 1.00 | 1.00 | 1.00 | 0.00 | 0.00 | 0.00 | 1.00 | 0.00 | 1.00 |
| Median | 1.00 | 1.00 | 1.00 | 1.00 | 1.00 | 0.00 | 0.00 | 0.00 | 1.00 | 0.00 | 1.00 |
| Maximum | 1.00 | 1.00 | 1.00 | 1.00 | 1.00 | 0.00 | 0.00 | 0.00 | 1.00 | 1.00 | 1.00 |
| Day 2, | | | | | | | | | | | |
| evening | | | | | | | | | | | |
| N | 3 | 6 | 6 | 3 | 3 | 3 | 3 | 1 | 6 | 6 | 6 |
| Mean | 1.00 | 0.83 | 1.00 | 1.00 | 1.00 | 0.33 | 0.67 | 1.00 | 1.00 | 0.33 | 1.00 |
| SE* | 0.00 | 0.17 | 0.00 | 0.00 | 0.00 | 0.33 | 0.33 | | 0.00 | 0.21 | 0.00 |
| Minimum | 1.00 | 0.00 | 1.00 | 1.00 | 1.00 | 0.00 | 0.00 | 1.00 | 1.00 | 0.00 | 1.00 |
| Median | 1.00 | 1.00 | 1.00 | 1.00 | 1.00 | 0.00 | 1.00 | 1.00 | 1.00 | 0.00 | 1.00 |
| Maximum | 1.00 | 1.00 | 1.00 | 1.00 | 1.00 | 1.00 | 1.00 | 1.00 | 1.00 | 1.00 | 1.00 |
| Day 3, | | | | | | | | | | | |
| morning | | | | | | | | | | | |
| N | 3 | 3 | 2 | 2 | — | 2 | 1 | 1 | 2 | 6 | 2 |
| Mean | 1.00 | 1.00 | 1.00 | 1.00 | — | 0.00 | 1.00 | 0.00 | 1.00 | 0.50 | 1.00 |
| SE* | 0.00 | 0.00 | 0.00 | 0.00 | — | 0.00 | | | 0.00 | 0.22 | 0.00 |
| Minimum | 1.00 | 1.00 | 1.00 | 1.00 | — | 0.00 | 1.00 | 0.00 | 1.00 | 0.00 | 1.00 |
| Median | 1.00 | 1.00 | 1.00 | 1.00 | — | 0.00 | 1.00 | 0.00 | 1.00 | 0.50 | 1.00 |
| Maximum | 1.00 | 1.00 | 1.00 | 1.00 | — | 0.00 | 1.00 | 0.00 | 1.00 | 1.00 | 1.00 |
| Day 3, | | | | | | | | | | | |
| evening | | | | | | | | | | | |
| N | 3 | 6 | 6 | 3 | 3 | 3 | 3 | 1 | 6 | 6 | 6 |
| Mean | 1.00 | 1.00 | 1.00 | 1.00 | 1.00 | 0.67 | 0.67 | 1.00 | 1.00 | 0.50 | 1.00 |
| SE* | 0.00 | 0.00 | 0.00 | 0.00 | 0.00 | 0.33 | 0.33 | | 0.00 | 0.22 | 0.00 |
| Minimum | 1.00 | 1.00 | 1.00 | 1.00 | 1.00 | 0.00 | 0.00 | 1.00 | 1.00 | 0.00 | 1.00 |
| Median | 1.00 | 1.00 | 1.00 | 1.00 | 1.00 | 1.00 | 1.00 | 1.00 | 1.00 | 0.50 | 1.00 |
| Maximum | 1.00 | 1.00 | 1.00 | 1.00 | 1.00 | 1.00 | 1.00 | 1.00 | 1.00 | 1.00 | 1.00 |
| Day 4, | | | | | | | | | | | |
| morning | | | | | | | | | | | |
| N | 2 | — | — | 1 | — | 2 | 1 | 1 | 2 | 4 | 6 |
| Mean | 1.00 | — | — | 1.00 | — | 1.00 | 1.00 | 1.00 | 1.00 | 0.75 | 1.00 |
| SE* | 0.00 | — | — | | — | 0.00 | | | 0.00 | 0.25 | 0.00 |
| Minimum | 1.00 | — | — | 1.00 | — | 1.00 | 1.00 | 1.00 | 1.00 | 0.00 | 1.00 |
| Median | 1.00 | — | — | 1.00 | — | 1.00 | 1.00 | 1.00 | 1.00 | 1.00 | 1.00 |
| Maximum | 1.00 | — | — | 1.00 | — | 1.00 | 1.00 | 1.00 | 1.00 | 1.00 | 1.00 |
| Day 4, | | | | | | | | | | | |
| evening | | | | | | | | | | | |
| N | 3 | 6 | 6 | 3 | 3 | 3 | 3 | 1 | 6 | 6 | 6 |
| Mean | 1.00 | 1.00 | 1.00 | 1.00 | 1.00 | 1.00 | 1.00 | 1.00 | 1.00 | 0.83 | 1.00 |
| SE* | 0.00 | 0.00 | 0.00 | 0.00 | 0.00 | 0.00 | 0.00 | | 0.00 | 0.17 | 0.00 |
| Minimum | 1.00 | 1.00 | 1.00 | 1.00 | 1.00 | 1.00 | 1.00 | 1.00 | 1.00 | 0.00 | 1.00 |
| Median | 1.00 | 1.00 | 1.00 | 1.00 | 1.00 | 1.00 | 1.00 | 1.00 | 1.00 | 1.00 | 1.00 |
| Maximum | 1.00 | 1.00 | 1.00 | 1.00 | 1.00 | 1.00 | 1.00 | 1.00 | 1.00 | 1.00 | 1.00 |
| Day 5, | | | | | | | | | | | |
| morning | | | | | | | | | | | |
| N | — | — | — | — | — | 1 | — | — | — | 3 | — |
| Mean | — | — | — | — | — | 1.00 | — | — | — | 1.00 | — |
| SE* | — | — | — | — | — | | — | — | — | 0.00 | — |
| Minimum | — | — | — | — | — | 1.00 | — | — | — | 1.00 | — |
| Median | — | — | — | — | — | 1.00 | — | — | — | 1.00 | — |
| Maximum | — | — | — | — | — | 1.00 | — | — | — | 1.00 | — |
| Day 5, | | | | | | | | | | | |
| evening | | | | | | | | | | | |
| N | — | — | — | — | — | — | — | — | 5 | 6 | — |
| Mean | — | — | — | — | — | — | — | — | 1.00 | 1.00 | — |
| SE* | — | — | — | — | — | — | — | — | 0.00 | 0.00 | — |
| Minimum | — | — | — | — | — | — | — | — | 1.00 | 1.00 | — |
| Median | — | — | — | — | — | — | — | — | 1.00 | 1.00 | — |
| Maximum | — | — | — | — | — | — | — | — | 1.00 | 1.00 | — |

Baseline = Day 1, pre-injection.
*SE = Standard Error.
Somesthetic Test: 0 = not feel a change in temperature; 1 = feel a change in temperature.

Temperature perception block, as defined by the response to somesthetic testing, was achieved within 6 hours of administration of EDLA or IDLA formulations. In some instances, temperature perception block was achieved within 1 to 3 hours. The return to normal sensation varied between 1 to 4 days. Time to maximum effect varied as well, with maximum effect being achieved between 2 hours and 2 days after administration of EDLA or IDLA formulations.

Onset of temperature perception block was defined as the first time at which the subject did not feel a change in temperature. Like the analgesia/anesthesia data, the time to onset of temperature perception block results revealed that, for the most part, subjects in the 40K EDLA groups had a shorter onset than in the 120K groups. As can be seen in Table C8, more subjects in the 40K groups experienced blockade of temperature perception within 3-6 hours (83% across doses) compared to subjects in the 120K EDLA groups (11% across doses). Aqueous bupivacaine 0.25% had the most rapid onset of temperature perception block, with 89% of subjects experiencing onset within 1 hour.

As with the onset of analgesia/anesthesia, 2.5% 40K EDLA was the most effective dose in terms of time to onset, with 100% of subjects demonstrating block of temperature perception within 2 hours (vs. 0.312%=0, 0.625%=17%, 1.25%=33%). The maximal dose of 40K EDLA, 5.0%, had an onset slightly longer than that of the other 40K groups, with 50% of subjects showing temperature perception block within the first 6 hours and the other 50% in the next 6 hours.

The 40K EDLA formulation had a more rapid onset of temperature perception block than 40K IDLA (40K EDLA=33% within 1 hour; 40K IDLA=17% within 1 hour). The single subject in the IDLA 120K group did not demonstrate temperature perception blockade until after 12 hours post-injection. The results of these data were similar to the analgesia/anesthesia results and suggest that administration of EDLA to these intercostal nerves in the selected doses produces a more rapid onset of action than IDLA.

TABLE C8
Onset of and Duration of Temperature Perception Block

| | Study Part 1 | | | |
|---|---|---|---|---|
| | EDLA 120K | EDLA 120K | EDLA 120K | AB* 0.25% |
| Time to Onset | 0.625% (N = 3) | 1.25% (N = 3) | 2.5% (N = 3) | (N = 9) |
| ≦30 min. | 0 | 0 | 0 | 9(100%) |
| >30 min. <1 h | 0 | 0 | 0 | 0 |
| >1-2 h | 0 | 0 | 0 | 0 |
| >2-3 h | 0 | 0 | 0 | 0 |
| >3-6 h | 0 | 1 (33%) | 0 | 0 |
| >6-12 h | 0 | 1 (33%) | 1 (33%) | 0 |
| >12 h | 0 | 1 (33%) | 1 (33%) | 0 |
| Mean Duration [h] (SE) | 0 | 70.0 (5.3) | 36.0 (36.0) | 9.5 (1.6) |
| Range | 0 | 72.0-78.0 | 36.0-72.0 | 8.5-17.5 |

| | EDLA 40K | EDLA 40K | EDLA 40K | EDLA 40K | AB* |
|---|---|---|---|---|---|
| | 0.312% | 0.625% | 1.25% | 2.5% | 0.25% |
| Time to Onset | (N = 3) | (N = 6) | (N = 6) | (N = 3) | (N = 18) |
| ≦30 min. | 0 | 0 | 0 | 0 | 14 (78%) |
| >30 min. <1 h | 0 | 1 (17%) | 1 (17%) | 0 | 1 (6%) |
| >1-2 h | 0 | 0 | 1 (17%) | 3 (100%) | 2 (11%) |
| >2-3 h | 0 | 1 (17%) | 2 (33%) | 0 | 0 |
| >3-6 h | 1 (33%) | 3 (50%) | 2 (33%) | 0 | 0 |
| >6-12 h | 1 (33%) | 0 | 0 | 0 | 0 |
| >12 h | 1 (33%) | 0 | 0 | 0 | 0 |
| Mean Duration [h] (SE) | 3 (1.7) | 26.5 (6.7) | 24.5 (3.9) | 40.0 (0) | 8.7 (1.1) |
| Range | 3.0-6.0 | 28.0-42.5 | 25.5-39.0 | 40.0-40.0 | 8.5-17.5 |
| | | Study Part 2 | | | Study Part 3 |
| | EDLA 120K | IDLA 120K | EDLA 40K | IDLA 40K | 40K EDLA |
| | 1.25% | 1.25% | 2.5% | 2.5% | 5.0% |
| Time to Onset | (N = 2) | (N = 1) | (N = 6) | (N = 6) | (N = 6) |
| ≦30 min. | 0 | 0 | 1 (17%) | 0 | 1 (17%) |
| >30 min. <1 h | 0 | 0 | 1 (17%) | 1 (17%) | 0 |
| >1-2 h | 0 | 0 | 0 | 1 (17%) | 1 (17%) |
| >2-3 h | 0 | 0 | 0 | 1 (17%) | 0 |
| >3-6 h | 0 | 0 | 2 (33%) | 1 (17%) | 1 (17%) |
| >6-12 h | 0 | 0 | 1 (17%) | 0 | 3 (50%) |
| >12 h | 0 | 1 (100%) | 1 (17%) | 0 | 0 |
| Mean Duration | 0 | 0 | 32.2 (8.7) | 13.5 (4.5) | 60.0 (8.1) |
| [h] (SE) | | | | | |
| Range | 0 | 0 | 30.0-68.0 | 13.5-24.5 | 58.0-94.0 |

As can be seen in Table C8, the duration of temperature perception block was greater in the 120K EDLA groups (53 hours overall for patients who experienced temperature perception block) relative to the 40K EDLA groups (31 hours) across doses. This effect was true primarily for the 120K 1.25% group, in which the mean duration of temperature perception block was 70 hours. This was longer than any other group. As with the analgesia/anesthesia results, aqueous bupivacaine had a relatively short mean duration of action (about 9 hours).

Within the 40K EDLA groups, a dose-effect relationship was observed, with the lowest dose producing the shortest duration of temperature perception block and the highest dose the longest duration (0.312%=3 hrs, 0.625%=27 hrs, 1.25%=25 hrs, 2.5%=40 The relationship was less clear for the 120K EDLA groups (0.625%=no blocks, 1.25%=70 hrs, 2.5%=36 hrs). These results demonstrate that EDLA has a longer-lasting effect than aqueous bupivacaine.

As FIG. C6 and Table C8 show, duration of temperature perception block was longer in the 40K EDLA 2.5% group (32 hours) than in the 40K IDLA 2.5% group (13.5 hours), supporting the notion that the addition of dexamethasone to the EDLA formulation extends its duration of action. The maximal 40K EDLA dose, 5.0%, produced a mean duration of temperature perception block that was longer than that seen in any other 40K EDLA group (60 hours), although not as long as the longest duration seen in the Study Part 1 120K group (1.25%=70 hours).

Decree of Numbness

Analgesia and anesthesia, assessed as the degree of numbness on a scale of 0-10, with 0 =not numb, and 10=totally numb, is shown in Table C9 below and FIGS. C7-C 10, as a function of time after administration of EDLA or IDLA formulations or aqueous bupivacaine.

TABLE C9
Degree of Numbness Results vs. Time
40K EDLA, 120K EDLA and IDLA

| | 40K | 40K | 40K | 40K | 120K | 120K | 120K | 120K | 40K | 40K | Aq. |
|---|---|---|---|---|---|---|---|---|---|---|---|
| | EDLA | EDLA | EDLA | EDLA | EDLA | EDLA | EDLA | IDLA | IDLA | EDLA | Bup. |
| | 0.312% | 0.625% | 1.25% | 2.5% | 0.625% | 1.25% | 2.50% | 1.25% | 2.5% | 5.0% | 0.5% |
| Baseline | | | | | | | | | | | |
| N | 3 | 6 | 6 | 3 | 3 | 3 | 3 | 1 | 6 | 6 | 6 |
| Mean | 0.00 | 0.00 | 0.00 | 0.00 | 0.00 | 0.00 | 0.00 | 0.00 | 0.00 | 0.00 | 0.00 |
| SE* | 0.00 | 0.00 | 0.00 | 0.00 | 0.00 | 0.00 | 0.00 | | 0.00 | 0.00 | 0.00 |
| Minimum | 0.00 | 0.00 | 0.00 | 0.00 | 0.00 | 0.00 | 0.00 | 0.00 | 0.00 | 0.00 | 0.00 |
| Median | 0.00 | 0.00 | 0.00 | 0.00 | 0.00 | 0.00 | 0.00 | 0.00 | 0.00 | 0.00 | 0.00 |
| Maximum | 0.00 | 0.00 | 0.00 | 0.00 | 0.00 | 0.00 | 0.00 | 0.00 | 0.00 | 0.00 | 0.00 |
| Hour 0.5 | | | | | | | | | | | |
| N | 3 | 6 | 6 | 3 | 3 | 3 | 3 | 1 | 6 | 6 | 6 |
| Mean | 0.00 | 0.33 | 1.50 | 1.00 | 0.00 | 0.00 | 0.00 | 0.00 | 0.67 | 2.83 | 8.00 |
| SE* | 0.00 | 0.33 | 1.15 | 1.00 | 0.00 | 0.00 | 0.00 | | 0.33 | 1.30 | 0.63 |
| Minimum | 0.00 | 0.00 | 0.00 | 0.00 | 0.00 | 0.00 | 0.00 | 0.00 | 0.00 | 0.00 | 6.00 |
| Median | 0.00 | 0.00 | 0.00 | 0.00 | 0.00 | 0.00 | 0.00 | 0.00 | 0.50 | 2.00 | 8.00 |
| Maximum | 0.00 | 2.00 | 7.00 | 3.00 | 0.00 | 0.00 | 0.00 | 0.00 | 2.00 | 9.00 | 10.00 |
| Hour 1 | | | | | | | | | | | |
| N | 3 | 6 | 6 | 3 | 3 | 3 | 3 | 1 | 6 | 6 | 6 |
| Mean | 0.00 | 0.33 | 3.17 | 3.33 | 0.00 | 0.00 | 0.00 | 0.00 | 1.50 | 6.33 | 9.17 |
| SE* | 0.00 | 0.33 | 2.01 | 0.88 | 0.00 | 0.00 | 0.00 | | 0.62 | 1.65 | 0.31 |
| Minimum | 0.00 | 0.00 | 0.00 | 2.00 | 0.00 | 0.00 | 0.00 | 0.00 | 0.00 | 0.00 | 8.00 |
| Median | 0.00 | 0.00 | 0.00 | 3.00 | 0.00 | 0.00 | 0.00 | 0.00 | 1.50 | 7.50 | 9.00 |
| Maximum | 0.00 | 2.00 | 10.00 | 5.00 | 0.00 | 0.00 | 0.00 | 0.00 | 4.00 | 9.00 | 10.00 |
| Hour 2 | | | | | | | | | | | |
| N | 3 | 6 | 6 | 3 | 3 | 3 | 3 | 1 | 6 | 6 | 6 |
| Mean | 0.00 | 2.33 | 4.00 | 8.67 | 0.00 | 0.00 | 0.00 | 0.00 | 2.67 | 7.33 | 9.33 |
| SE* | 0.00 | 1.05 | 2.00 | 1.33 | 0.00 | 0.00 | 0.00 | | 0.92 | 1.67 | 0.33 |
| Minimum | 0.00 | 0.00 | 0.00 | 6.00 | 0.00 | 0.00 | 0.00 | 0.00 | 0.00 | 0.00 | 8.00 |
| Median | 0.00 | 1.50 | 2.00 | 10.00 | 0.00 | 0.00 | 0.00 | 0.00 | 3.00 | 9.50 | 9.50 |
| Maximum | 0.00 | 6.00 | 10.00 | 10.00 | 0.00 | 0.00 | 0.00 | 0.00 | 5.00 | 10.00 | 10.00 |
| Hour 3 | | | | | | | | | | | |
| N | 3 | 6 | 6 | 3 | 3 | 3 | 3 | 1 | 6 | 6 | 6 |
| Mean | 0.00 | 4.67 | 5.67 | 10.00 | 0.00 | 0.00 | 0.00 | 0.00 | 4.50 | 7.83 | 9.67 |
| SE* | 0.00 | 1.50 | 1.94 | 0.00 | 0.00 | 0.00 | 0.00 | | 1.50 | 1.58 | 0.21 |
| Minimum | 0.00 | 0.00 | 0.00 | 10.00 | 0.00 | 0.00 | 0.00 | 0.00 | 0.00 | 0.00 | 9.00 |
| Median | 0.00 | 5.00 | 7.00 | 10.00 | 0.00 | 0.00 | 0.00 | 0.00 | 5.50 | 9.00 | 10.00 |
| Maximum | 0.00 | 9.00 | 10.00 | 10.00 | 0.00 | 0.00 | 0.00 | 0.00 | 8.00 | 10.00 | 10.00 |
| Hour 6 | | | | | | | | | | | |
| N | 3 | 6 | 6 | 3 | 3 | 3 | 3 | 1 | 6 | 6 | 6 |
| Mean | 1.33 | 6.33 | 8.17 | 9.67 | 0.00 | 2.67 | 0.00 | 0.00 | 6.17 | 8.67 | 9.17 |
| SE* | 1.33 | 1.58 | 1.14 | 0.33 | 0.00 | 2.19 | 0.00 | | 1.97 | 1.33 | 0.31 |
| Minimum | 0.00 | 0.00 | 3.00 | 9.00 | 0.00 | 0.00 | 0.00 | 0.00 | 0.00 | 2.00 | 8.00 |
| Median | 0.00 | 7.00 | 9.50 | 10.00 | 0.00 | 1.00 | 0.00 | 0.00 | 8.50 | 10.00 | 9.00 |
| Maximum | 4.00 | 10.00 | 10.00 | 10.00 | 0.00 | 7.00 | 0.00 | 0.00 | 10.00 | 10.00 | 10.00 |
| Hour 12 | | | | | | | | | | | |
| N | 3 | 6 | 6 | 3 | 3 | 3 | 3 | 1 | 6 | 6 | 6 |
| Mean | 2.67 | 7.33 | 10.00 | 9.33 | 0.00 | 4.67 | 1.67 | 0.00 | 4.83 | 8.67 | 1.33 |
| SE* | 2.19 | 1.50 | 0.00 | 0.67 | 0.00 | 2.33 | 1.67 | | 2.17 | 0.88 | 0.80 |
| Minimum | 0.00 | 0.00 | 10.00 | 8.00 | 0.00 | 0.00 | 0.00 | 0.00 | 0.00 | 5.00 | 0.00 |
| Median | 1.00 | 8.50 | 10.00 | 10.00 | 0.00 | 7.00 | 0.00 | 0.00 | 4.50 | 10.00 | 0.50 |
| Maximum | 7.00 | 10.00 | 10.00 | 10.00 | 0.00 | 7.00 | 5.00 | 0.00 | 10.00 | 10.00 | 5.00 |
| Day 1, | | | | | | | | | | | |
| morning | | | | | | | | | | | |
| N | 3 | 6 | 6 | 3 | 3 | 3 | 3 | 1 | 6 | 6 | 6 |
| Mean | 0.33 | 6.83 | 9.00 | 8.67 | 0.00 | 4.33 | 1.67 | 0.00 | 2.00 | 7.83 | 0.33 |
| SE* | 0.33 | 1.38 | 0.82 | 1.33 | 0.00 | 2.33 | 1.67 | | 1.26 | 1.01 | 0.33 |
| Minimum | 0.00 | 0.00 | 5.00 | 6.00 | 0.00 | 0.00 | 0.00 | 0.00 | 0.00 | 5.00 | 0.00 |
| Median | 0.00 | 8.00 | 10.00 | 10.00 | 0.00 | 5.00 | 0.00 | 0.00 | 0.00 | 8.50 | 0.00 |
| Maximum | 1.00 | 9.00 | 10.00 | 10.00 | 0.00 | 8.00 | 5.00 | 0.00 | 6.00 | 10.00 | 2.00 |
| Day 1, | | | | | | | | | | | |
| evening | | | | | | | | | | | |
| N | 3 | 6 | 6 | 3 | 3 | 3 | 3 | 1 | 6 | 6 | 6 |
| Mean | 0.00 | 3.17 | 2.83 | 8.33 | 0.00 | 3.67 | 1.33 | 3.00 | 0.83 | 6.67 | 1.50 |
| SE* | 0.00 | 1.11 | 1.64 | 1.20 | 0.00 | 1.33 | 1.33 | | 0.83 | 0.95 | 1.50 |
| Minimum | 0.00 | 0.00 | 0.00 | 6.00 | 0.00 | 1.00 | 0.00 | 3.00 | 0.00 | 3.00 | 0.00 |
| Median | 0.00 | 2.50 | 1.00 | 9.00 | 0.00 | 5.00 | 0.00 | 3.00 | 0.00 | 6.50 | 0.00 |
| Maximum | 0.00 | 8.00 | 10.00 | 10.00 | 0.00 | 5.00 | 4.00 | 3.00 | 5.00 | 10.00 | 9.00 |
| Day 2, | | | | | | | | | | | |
| morning | | | | | | | | | | | |
| N | 3 | 6 | 6 | 3 | 1 | 2 | 1 | 1 | 3 | 6 | 6 |
| Mean | 0.33 | 0.17 | 1.83 | 4.00 | 0.00 | 4.50 | 3.00 | 7.00 | 0.00 | 4.83 | 0.00 |
| SE* | 0.33 | 0.17 | 1.47 | 1.00 | | 0.50 | | | 0.00 | 1.11 | 0.00 |
| Minimum | 0.00 | 0.00 | 0.00 | 3.00 | 0.00 | 4.00 | 3.00 | 7.00 | 0.00 | 3.00 | 0.00 |
| Median | 0.00 | 0.00 | 0.00 | 3.00 | 0.00 | 4.50 | 3.00 | 7.00 | 0.00 | 4.00 | 0.00 |
| Maximum | 1.00 | 1.00 | 9.00 | 6.00 | 0.00 | 5.00 | 3.00 | 7.00 | 0.00 | 10.00 | 0.00 |
| Day 2, | | | | | | | | | | | |
| evening | | | | | | | | | | | |
| N | 3 | 6 | 6 | 3 | 3 | 3 | 3 | 1 | 6 | 6 | 6 |
| Mean | 0.00 | 0.33 | 0.83 | 1.00 | 0.00 | 2.67 | 1.33 | 10.00 | 0.00 | 4.33 | 0.33 |
| SE* | 0.00 | 0.33 | 0.83 | 1.00 | 0.00 | 1.45 | 1.33 | | 0.00 | 0.92 | 0.33 |
| Minimum | 0.00 | 0.00 | 0.00 | 0.00 | 0.00 | 1.00 | 0.00 | 10.00 | 0.00 | 2.00 | 0.00 |
| Median | 0.00 | 0.00 | 0.00 | 0.00 | 0.00 | 3.00 | 0.00 | 10.00 | 0.00 | 3.50 | 0.00 |
| Maximum | 0.00 | 2.00 | 5.00 | 3.00 | 0.00 | 5.00 | 4.00 | 10.00 | 0.00 | 8.00 | 2.00 |
| Day 3, | | | | | | | | | | | |
| morning | | | | | | | | | | | |
| N | 3 | 3 | 2 | 2 | — | 2 | 1 | 1 | 2 | 6 | 3 |
| Mean | 0.00 | 0.00 | 1.50 | 0.00 | — | 4.00 | 3.00 | 10.00 | 0.00 | 3.17 | 0.00 |
| SE* | 0.00 | 0.00 | 1.50 | 0.00 | — | 1.00 | | | 0.00 | 1.19 | 0.00 |
| Minimum | 0.00 | 0.00 | 0.00 | 0.00 | — | 3.00 | 3.00 | 10.00 | 0.00 | 0.00 | 0.00 |
| Median | 0.00 | 0.00 | 1.50 | 0.00 | — | 4.50 | 3.00 | 10.00 | 0.00 | 2.50 | 0.00 |
| Maximum | 0.00 | 0.00 | 3.00 | 0.00 | — | 5.00 | 3.00 | 10.00 | 0.00 | 8.00 | 0.00 |
| Day 3, | | | | | | | | | | | |
| evening | | | | | | | | | | | |
| N | 3 | 6 | 6 | 3 | 3 | 3 | 3 | 1 | 6 | 6 | 6 |
| Mean | 0.00 | 0.00 | 0.33 | 0.33 | 0.00 | 1.33 | 0.67 | 7.00 | 0.00 | 2.83 | 0.00 |
| SE* | 0.00 | 0.00 | 0.33 | 0.33 | 0.00 | 0.88 | 0.67 | | 0.00 | 1.25 | 0.00 |
| Minimum | 0.00 | 0.00 | 0.00 | 0.00 | 0.00 | 0.00 | 0.00 | 7.00 | 0.00 | 0.00 | 0.00 |
| Median | 0.00 | 0.00 | 0.00 | 0.00 | 0.00 | 1.00 | 0.00 | 7.00 | 0.00 | 1.50 | 0.00 |
| Maximum | 0.00 | 0.00 | 2.00 | 1.00 | 0.00 | 3.00 | 2.00 | 7.00 | 0.00 | 8.00 | 0.00 |
| Day 4, | | | | | | | | | | | |
| morning | | | | | | | | | | | |
| N | 2 | — | — | 1 | — | 2 | 1 | 1 | 2 | 4 | — |
| Mean | 0.00 | — | — | 0.00 | — | 0.50 | 0.00 | 0.00 | 0.00 | 3.00 | — |
| SE* | 0.00 | — | — | | — | 0.50 | | | 0.00 | 1.08 | — |
| Minimum | 0.00 | — | — | 0.00 | — | 0.00 | 0.00 | 0.00 | 0.00 | 1.00 | — |
| Median | 0.00 | — | — | 0.00 | — | 0.50 | 0.00 | 0.00 | 0.00 | 2.50 | — |
| Maximum | 0.00 | — | — | 0.00 | — | 1.00 | 0.00 | 0.00 | 0.00 | 6.00 | — |
| Day 4, | | | | | | | | | | | |
| evening | | | | | | | | | | | |
| N | 3 | 6 | 6 | 3 | 3 | 3 | 3 | 1 | 6 | 6 | 6 |
| Mean | 0.00 | 0.00 | 0.00 | 0.00 | 0.00 | 0.33 | 0.00 | 3.00 | 0.00 | 2.33 | 0.00 |
| SE* | 0.00 | 0.00 | 0.00 | 0.00 | 0.00 | 0.33 | 0.00 | | 0.00 | 1.31 | 0.00 |
| Minimum | 0.00 | 0.00 | 0.00 | 0.00 | 0.00 | 0.00 | 0.00 | 3.00 | 0.00 | 0.00 | 0.00 |
| Median | 0.00 | 0.00 | 0.00 | 0.00 | 0.00 | 0.00 | 0.00 | 3.00 | 0.00 | 1.00 | 0.00 |
| Maximum | 0.00 | 0.00 | 0.00 | 0.00 | 0.00 | 1.00 | 0.00 | 3.00 | 0.00 | 8.00 | 0.00 |
| Day 5, | | | | | | | | | | | |
| morning | | | | | | | | | | | |
| N | — | — | — | — | — | 1 | — | — | — | 3 | — |
| Mean | — | — | — | — | — | 0.00 | — | — | — | 2.33 | — |
| SE* | — | — | — | — | — | | — | — | — | 2.33 | — |
| Minimum | — | — | — | — | — | 0.00 | — | — | — | 0.00 | — |
| Median | — | — | — | — | — | 0.00 | — | — | — | 0.00 | — |
| Maximum | — | — | — | — | — | 0.00 | — | — | — | 7.00 | — |
| Day 5, | | | | | | | | | | | |
| evening | | | | | | | | | | | |
| N | — | — | — | — | 3 | 3 | 3 | 1 | 5 | 6 | — |
| Mean | — | — | — | — | 0.00 | 0.33 | 0.00 | 0.00 | 0.00 | 1.17 | — |
| SE* | — | — | — | — | 0.00 | 0.33 | 0.00 | | 0.00 | 2.17 | — |
| Minimum | — | — | — | — | 0.00 | 0.00 | 0.00 | 0.00 | 0.00 | 0.00 | — |
| Median | — | — | — | — | 0.00 | 0.00 | 0.00 | 0.00 | 0.00 | 0.00 | — |
| Maximum | — | — | — | — | 0.00 | 1.00 | 0.00 | 0.00 | 0.00 | 7.00 | — |
| Day 6, | | | | | | | | | | | |
| evening | | | | | | | | | | | |
| N | — | — | — | — | 3 | 3 | 3 | — | — | 1 | — |
| Mean | — | — | — | — | 0.00 | 0.33 | 0.00 | — | — | 3.00 | — |
| SE* | — | — | — | — | 0.00 | 0.33 | 0.00 | — | — | | — |
| Minimum | — | — | — | — | 0.00 | 0.00 | 0.00 | — | — | 3.00 | — |
| Median | — | — | — | — | 0.00 | 0.00 | 0.00 | — | — | 3.00 | — |
| Maximum | — | — | — | — | 0.00 | 1.00 | 0.00 | — | — | 3.00 | — |
| Day 7, | | | | | | | | | | | |
| evening | | | | | | | | | | | |
| N | — | — | — | — | 3 | 3 | 3 | 1 | 5 | 6 | — |
| Mean | — | — | — | — | 0.00 | 1.67 | 0.00 | 0.00 | 0.00 | 0.50 | — |
| SE* | — | — | — | — | 0.00 | 1.67 | 0.00 | | 0.00 | 0.50 | — |
| Minimum | — | — | — | — | 0.00 | 0.00 | 0.00 | 0.00 | 0.00 | 0.00 | — |
| Median | — | — | — | — | 0.00 | 0.00 | 0.00 | 0.00 | 0.00 | 0.00 | — |
| Maximum | — | — | — | — | 0.00 | 5.00 | 0.00 | 0.00 | 0.00 | 3.00 | — |
| Day 8, | | | | | | | | | | | |
| morning | | | | | | | | | | | |
| N | — | — | — | — | — | 1 | 1 | — | — | — | — |
| Mean | — | — | — | — | — | 6.00 | 4.00 | — | — | — | — |
| SE* | — | — | — | — | — | | | — | — | — | — |
| Minimum | — | — | — | — | — | 6.00 | 4.00 | — | — | — | — |
| Median | — | — | — | — | — | 6.00 | 4.00 | — | — | — | — |
| Maximum | — | — | — | — | — | 6.00 | 4.00 | — | — | — | — |
| Day 8, | | | | | | | | | | | |
| evening | | | | | | | | | | | |
| N | — | — | — | — | 3 | 3 | 3 | — | — | — | — |
| Mean | — | — | — | — | 0.00 | 1.67 | 0.67 | — | — | — | — |
| SE* | — | — | — | — | 0.00 | 1.67 | 0.67 | — | — | — | — |
| Minimum | — | — | — | — | 0.00 | 0.00 | 0.00 | — | — | — | — |
| Median | — | — | — | — | 0.00 | 0.00 | 0.00 | — | — | — | — |
| Maximum | — | — | — | — | 0.00 | 5.00 | 2.00 | — | — | — | — |
| Day 9, | | | | | | | | | | | |
| morning | | | | | | | | | | | |
| N | — | — | — | — | — | 1 | 2 | — | — | — | — |
| Mean | — | — | — | — | — | 5.00 | 3.50 | — | — | — | — |
| SE* | — | — | — | — | — | | 0.50 | — | — | — | — |
| Minimum | — | — | — | — | — | 5.00 | 3.00 | — | — | — | — |
| Median | — | — | — | — | — | 5.00 | 3.50 | — | — | — | — |
| Maximum | — | — | — | — | — | 5.00 | 4.00 | — | — | — | — |
| Day 9, | | | | | | | | | | | |
| evening | | | | | | | | | | | |
| N | — | — | — | — | 3 | 3 | 3 | — | — | — | — |
| Mean | — | — | — | — | 0.00 | 1.33 | 1.33 | — | — | — | — |
| SE* | — | — | — | — | 0.00 | 1.33 | 0.88 | — | — | — | — |
| Minimum | — | — | — | — | 0.00 | 0.00 | 0.00 | — | — | — | — |
| Median | — | — | — | — | 0.00 | 0.00 | 1.00 | — | — | — | — |
| Maximum | — | — | — | — | 0.00 | 4.00 | 3.00 | — | — | — | — |
| Day 10, | | | | | | | | | | | |
| morning | | | | | | | | | | | |
| N | — | — | — | — | — | 1 | 1 | — | — | — | — |
| Mean | — | — | — | — | — | 3.00 | 3.00 | — | — | — | — |
| SE* | — | — | — | — | — | | | — | — | — | — |
| Minimum | — | — | — | — | — | 3.00 | 3.00 | — | — | — | — |
| Median | — | — | — | — | — | 3.00 | 3.00 | — | — | — | — |
| Maximum | — | — | — | — | — | 3.00 | 3.00 | — | — | — | — |
| Day 10, | | | | | | | | | | | |
| evening | | | | | | | | | | | |
| N | — | — | — | — | 2 | 3 | 3 | — | — | — | — |
| Mean | — | — | — | — | 0.00 | 0.33 | 1.00 | — | — | — | — |
| SE* | — | — | — | — | 0.00 | 0.33 | 1.00 | — | — | — | — |
| Minimum | — | — | — | — | 0.00 | 0.00 | 0.00 | — | — | — | — |
| Median | — | — | — | — | 0.00 | 0.00 | 0.00 | — | — | — | — |
| Maximum | — | — | — | — | 0.00 | 1.00 | 3.00 | — | — | — | — |
| Day 11, | | | | | | | | | | | |
| morning | | | | | | | | | | | |
| N | — | — | — | — | — | — | 1 | — | — | — | — |
| Mean | — | — | — | — | — | — | 3.00 | — | — | — | — |
| SE* | — | — | — | — | — | — | | — | — | — | — |
| Minimum | — | — | — | — | — | — | 3.00 | — | — | — | — |
| Median | — | — | — | — | — | — | 3.00 | — | — | — | — |
| Maximum | — | — | — | — | — | — | 3.00 | — | — | — | — |
| Day 11, | | | | | | | | | | | |
| evening | | | | | | | | | | | |
| N | — | — | — | — | 3 | 3 | 3 | — | — | — | — |
| Mean | — | — | — | — | 0.00 | 0.00 | 1.00 | — | — | — | — |
| SE* | — | — | — | — | 0.00 | 0.00 | 1.00 | — | — | — | — |
| Minimum | — | — | — | — | 0.00 | 0.00 | 0.00 | — | — | — | — |
| Median | — | — | — | — | 0.00 | 0.00 | 0.00 | — | — | — | — |
| Maximum | — | — | — | — | 0.00 | 0.00 | 3.00 | — | — | — | — |
| Day 12, | | | | | | | | | | | |
| evening | | | | | | | | | | | |
| N | — | — | — | — | 3 | 3 | 3 | — | — | — | — |
| Mean | — | — | — | — | 0.00 | 0.00 | 0.00 | — | — | — | — |
| SE* | — | — | — | — | 0.00 | 0.00 | 0.00 | — | — | — | — |
| Minimum | — | — | — | — | 0.00 | 0.00 | 0.00 | — | — | — | — |
| Median | — | — | — | — | 0.00 | 0.00 | 0.00 | — | — | — | — |
| Maximum | — | — | — | — | 0.00 | 0.00 | 0.00 | — | — | — | — |

Baseline = Day 1, pre-injection.
*SE = Standard Error.
Numbness scale of 0-10: 0 = not numb; 10 = totally numb.

Degree of numbness, as defined on a scale of numbness of 0-10, was achieved within 6 hours of administration of EDLA or IDLA formulations. In some instances, numbness was achieved within 1 to 3 hours. Return to normal sensation varied between 1 to 4 days.

FIGS. C7, C8 and C10 show the degree of numbness results over time for 40K and 120K EDLA. Mean numbness scores for the selected dose of 40K EDLA (2.5%) peaked earlier (3 hours post-injection) and higher (mean numbness score of 10) than the scores for the 120K EDLA selected dose (1.25%, peak numbness score of 4.7 at 12 hours post-injection).Within the 40K EDLA group, with the exception of the 2.5% selected dose, all dose groups had numbness scores that peaked at 12 hours post-injection. A dose-response relationship was observed with respect to the peak scores in the 40K EDLA group (0.312%=2.7, 0.625%=7.3, 1.25%=10,2.5%=10).

The dose-response relationship was less clear in the 120K EDLA dose groups (0.625%=no block, 1.25%=4.7,2.5%=1.7). Like the 40K formulation, the times to peak numbness were consistent at 12 hours post-injection. The earliest peak numbness score was seen in the active control group, 0.25% aqueous bupivacaine (peak numbness score of 9.2 at 2 hours post-injection). The 40K EDLA 2.5% dose group (complete numbness at 3 hours post-injection) showed an early, high peak that would be ideal for most applications of this drug. The 5% formulation of 40K EDLA produced a peak numbness score of 8.7, with onset at 6 hours (see FIG. C10).

With regard to the IDLA formulations, a peak numbness score of 10 was reported on Day 2 in the one subject receiving 120K IDLA 1.25%. The 40K IDLA 2.5% formulation had a shorter onset to peak (6 hours) and a smaller peak numbness score (6.2) relative to 40K EDLA 2.5% (peak numbness score of 7.8 at 12 hours post-injection) (FIG. C9). The 12-hour onset in the EDLA group in this part of the study is in contrast to the early peak (3 hours) seen in this dose group in Part 1 of the study. These data suggest higher (but later) peak numbness for 40K EDLA vs 40K IDLA.

Pharmacokinetic Results

The results are summarized by treatment group over time in Table C10, and presented graphically in FIGS. C11-C15. All plasma bupivacaine levels for EDLA, IDLA, and AB that resulted in effective analgesia/anesthesia in this study were well below those at which systemic toxic reactions are believed to occur (i.e., 4000 ng/mL) according to Moore, D. C., Mather, L. E., Bridenbaugh, P. O., “Arterial and venous plasma levels of bupivacaine following peripheral nerve block,” Anesth Analg. 1976, Vol. 55, pp. 763-68, incorporated by reference herein.

As FIGS. C11, C12 and C15 show, bupivacaine concentrations for 40K EDLA tended to be higher than those seen for 120K EDLA, especially in the 3 highest 40K EDLA dose groups (1.25%, 2.5%, 5.0%). Both 1.25% 40K EDLA and 2.5% 40K EDLA demonstrated an early peak in plasma concentrations (approximately 15 minutes post-injection), which was similar to that seen in the 120K dose groups, as well as a second peak occurring approximately 6 to 12 hours post-injection. The second peak was not observed with 120K EDLA. While the precise reason for this second peak is not known, it suggests that the initial early peak is due to release of aqueous bupivacaine, administered as the control in this study, while the later peak is due to the sustained release of bupivacaine from EDLA microspheres. A second peak in dexamethasone levels was also observed at the same times for 1.25% 120K EDLA.

In Part 2, the comparison of 2.5% EDLA and IDLA 40K formulations showed only a small initial early peak in plasma bupivacaine concentration (about 50 ng/mL), as shown in FIG. C13. The high molecular weight formulations, 120K EDLA and IDLA at 1.25% showed only delayed peaks at about 48 hours of approximately 10 ng/mL and 40 ng/mL, respectively (FIG. C14). The 5.0% 40K EDLA dosing group (FIG. C15) did not show an early peak in concentrations, but rather, showed only a delayed peak (peak time=24 hours). These observations are further evidence that the initial early peak seen in the Part 1 studies is primarily due to release of aqueous bupivacaine administered as the control treatment.

In all 40K groups except 5.0%, plasma bupivacaine concentrations were back to baseline (undetectable) levels by 72 hours post-injection (96 hours in the 5.0% group). In the 120K EDLA groups, plasma bupivacaine concentrations were essentially back to baseline (pre-injection) undetectable levels by 96 hours post-injection, with the exception of some residual low levels (8.7 to 13.0 ng/mL) seen in the Part 1, 1.25% and 2.5% 120K EDLA groups only. These data suggest that the bupivacaine administered via 1.25% and 2.5% 120K EDLA remains in the body for longer periods of time than for all but the maximum dose of 40K EDLA.

For all doses of the 120K EDLA formulation, mean plasma dexamethasone levels were undetectable (zero) at the time points measured. Detectable dexamethasone concentrations were, however, evident in some of the 40K EDLA groups, (data not shown). Specifically, dexamethasone was detectable in plasma in the 3 highest dose groups (1.25%, 2.5%, and 5.0%). These levels peaked at approximately 6 to 12 hours post-injection (1.25% peak level=42.9 ng/mL; 2.5%=91.0 ng/mL and 103.2 ng/mL (in Parts 1 and 2,respectively); and 5.0%=196.8 ng/mL) and appeared to correlate with the second peak in plasma bupivacaine levels seen in the 1.25% and 2.5% 40K groups, shown in FIG. C11, and with the delayed peak seen in the 40K 5.0% group. No dexamethasone was detectable in any IDLA group.

Bupivacaine and Dexamethasone Pharmacokinetic Parameters

As can be seen in Table C10, the time of occurrence for peak bupivacaine concentrations (tₘₐₓ) was shorter in the 120K and 40K groups of Part 1 vs. Parts 2 and 3; most likely due to the concurrent administration of aqueous bupivacaine in Part 1. The maximum concentration of drug (Cₘₐₓ) was similar across doses both within and across the 120K and 40K EDLA groups (130.3 ng/dl and 167.0 ng/dL, respectively), with the exception of higher concentrations seen in the 2.5% 40K EDLA (167 ng/dl) and 5.0% 40K EDLA (227.8 ng/dl) groups. No Cₘₐₓ data were reported for the 1.25% 120K EDLA group in Part 2 of the study since no patient ever had detectable bupivacaine levels; however, in the 40K formulation, a higher Cₘₐₓ was observed in the IDLA group relative to EDLA.

For subjects in Part 1, the total bupivacaine AUC reflects bupivacaine from injections of both EDLA and AB. For the 4 tested concentrations of EDLA 40K, bupivacaine mean AUCₜs ranged from 1093 to 4087 ng/mL-hr; there was a direct relationship between the concentration of EDLA and bupivacaine AUC. The mean bupivacaine AUCₜ for subjects receiving 2.5% EDLA 40K in Part 2 (without simultaneous AB) was lower than for subjects receiving the same concentration of EDLA 40K in Part 1 (with simultaneous AB). Subjects in Part 3 (5.0% EDLA 40K, without AB) had the highest total AUC (7943 ng/mL-hr). Subjects who received 2.5% IDLA 40K in Part 2 had a higher mean bupivacaine AUC, than the subjects who received EDLA, presumably because of a higher mean Cₘₐₓ. Bupivacaine AUCₜs for the 9 subjects who received EDLA 120K and the subject who received IDLA 120K are also shown in Table C10.

TABLE C10
Pharmacokinetic Parameters for Plasma Bupivacaine

| | Study Part 1* | | | | | | |
|---|---|---|---|---|---|---|---|
| | EDLA | EDLA | EDLA | EDLA | EDLA | EDLA | EDLA |
| | 120K | 120K | 120K | 40K | 40K | 40K | 40K |
| | 0.625% | 1.25% | 2.5% | 0.312% | 0.625% | 1.25% | 2.5% |
| PK Parameter | (N = 3) | (N = 3) | (N = 3) | (N = 3) | (N = 6) | (N = 6) | (N = 3) |
| Tₘₐₓ (h) | 0.3(0) | 0.5(0.3) | 0.3(0) | 0.3(0) | 0.4(0.1) | 5.2(3.9) | 4.2(3.9) |
| Cₘₐₓ (ng/mL) | 130.3 | 122.7 | 101.3 | 127.0 | 116.0 | 126.5 | 167.0 |
| | (5.0) | (8.3) | (7.5) | (18.8) | (11.2) | (18.7) | (12.0) |
| AUCt | 625.6 | 2891.4 | 2351.1 | 1093.7 | 1588.9 | 2774.8 | 4087.2 |
| (ng/mL · h) | (141.0) | (460.7) | (229.3) | (139.6) | (382.9) | (559.3) | (456.2) |

| | Study Part 2 | | | | Study Part 3 |
|---|---|---|---|---|---|
| | EDLA 120K | IDLA 120K | EDLA 40K | IDLA 40K | 40K EDLA |
| PK Parameter | 1.25% (N = 2) | 1.25% (N = 1) | 2.5% (N = 6) | 2.5% (N = 6) | 5.0% (N = 6) |
| Tₘₐₓ (h) | — | 48.0 | 13.0(3.6) | 15.0(3.0) | 12.3(3.9) |
| Cₘₐₓ (ng/mL) | — | 35.4 | 101.6(9.7) | 164.9(28.3) | 227.8(31.9) |
| AUCt | — | 2140.1 | 3073.4(357.1) | 4583.1(1160.6) | 7943.8(1078.0) |
| (ng/mL · h) | | | | | |

*All subjects in Part 1 also received 0.25% aqueous bupivacaine
No plasma dexamethasone pharmacokinetic parameters were reported in Study Parts 1 or 2. In Part 3(40K EDLA 5.0%), plasma dexamethasone was associated with a mean tₘₐₓ of 7 hours and a Cₘₐₓ of 198 ng/dl.

Summary and Conclusions

Across dose groups, 40K EDLA had a faster onset of analgesia/anesthesia relative to 120K EDLA while aqueous bupivacaine had a more rapid onset than both EDLA groups (100% onset within 2 hours). The 2.5% 40K EDLA dose was the most effective (100% onset within 2 hours) followed by the 1.25% 120K EDLA dose (67% within 3-6 hours); although not as effective as 40K EDLA. EDLA produced a more rapid onset compared with IDLA (EDLA, 66% within 2 hours; IDLA, 50% within 2 hours).

The duration of analgesia/anesthesia in the selected doses (120K EDLA=1.25%, 40K EDLA=2.5%) from Study Part 1 was greater in the 120K EDLA group (68 hours) than the 40K EDLA group (35 hours). Aqueous bupivacaine had a relatively short duration of action (8-9 hours). Within dosing groups, a dose-effect relationship was apparent in both the 120K EDLA groups (1.25%=64 hours, 2.5%=75 hours) and the 40K EDLA groups (0.312%=5 hours, 0.625%=39 hours, 1.25%=43 hours; 2.5%=44 hours). With respect to the EDLA vs. IDLA comparison, EDLA had a longer duration of analgesic/anesthetic action relative to IDLA (EDLA=45 hours, IDLA=20 hours). These data suggest that dexamethasone prolongs the duration of analgesic/anesthetic action of EDLA.

All aqueous bupivacaine sensory blocks demonstrated only anesthesia without analgesia. The 40K EDLA formulation blocks produced the most analgesia (0.312%=67% of blocks, 0.625%=17% of blocks; 5.0%=17% of blocks). These findings suggest that, overall, EDLA is more likely to be associated with analgesia than aqueous bupivacaine and that the 40K dose formulation is more effective in this respect than the 120K formulation. In the EDLA vs. IDLA comparison, more analgesia was seen in EDLA subjects (50%) vs. IDLA (0%).

The onset of temperature perception block data closely resembled the analgesic/anesthetic data. The most rapid onset of temperature perception block was seen in the aqueous bupivacaine groups (89% within 1 hour). More subjects in the 40K EDLA groups (across doses) had onset within 3-6 hours vs. the 120K EDLA groups (11%). Within the 40K EDLA groups, the 2.5% dose group was the most effective (100% onset within 2 hours). 40K EDLA had a more rapid onset of temperature perception block compared with IDLA (33% within 1 hour vs. 17% within 1 hour).

The duration of temperature perception block was greater in the 120K EDLA groups (across doses) relative to the 40K EDLA groups (56 hours vs. 24 hours, respectively). Aqueous bupivacaine had the shortest duration (9 hours). EDLA also had a greater duration of temperature perception block than IDLA (32 hours vs. 13.5 hours, respectively), supporting the notion that dexamethasone increases the duration of action of aqueous bupivacaine.

At the selected dose (2.5%), 40K EDLA had a earlier time to peak numbness score and a higher peak score than the 120K EDLA selected dose [(1.25%) 40K=score of 10 at 3 hours post-injection; 120K=score of 4.7 at 12 hours post-injection). A dose-effect relationship in peak scores was observed in the 40K EDLA but not 120K EDLA groups. The earliest peak numbness score was seen in the aqueous bupivacaine groups (score of 9.2 at 2 hours post-injection). The EDLA and IDLA groups were similar with respect to onset of peak numbness score (EDLA=7.8 at hour 12; IDLA=6.2 at Hour 6).

Plasma bupivacaine concentrations in the three highest dose groups of 40K EDLA groups were higher than those seen in the 120K EDLA groups. The 1.25% and 2.5% 40K EDLA dose groups also showed an unexpected second peak in plasma levels occurring approximately 6-12 hours post-injection; this effect was not seen in any other group. In all but the highest 40K EDLA group, plasma bupivacaine levels were back to baseline (0) by 72 hours post-injection, whereas bupivacaine was still detectable in plasma in 120K EDLA-treated subjects by at least 96 hours post-injection. Importantly, all plasma bupivacaine levels for EDLA, IDLA, and AB that resulted in effective analgesia/anesthesia in this study were well below those at which systemic toxic reactions are believed to occur (i.e., 4000 ng/mL).

Plasma dexamethasone concentrations were undetectable in all subjects except the three highest 40K EDLA doses (1.25%, 2.5%, and 5.0%) in which plasma levels peaked at approximately 6-12 hours post-injection. These peaks appeared to correlate with the second peak in plasma bupivacaine levels seen in the 1.25% and 2.5% 40K groups and with the delayed peak seen in the 40K 5.0% group.

### SUPERFICIAL RADIAL NERVE EXAMPLES

### Example D

### EDLA with and Without Dexamethasone In Superficial Radial Nerve Block

A double-blind, randomized, 2-period crossover study evaluated the efficacy and safety of 2.5% 120K EDLA compared with 0.5% aqueous bupivacaine with dexamethasone (AB-D), each administered as a superficial radial nerve block.

The 120K EDLA (2.5%) suspension was prepared to yield a microsphere concentration of 2.5%, and supplying 18.75 mg bupivacaine and 10 microgram (μg) dexamethasone per mL. Three mL of 120K EDLA suspension was administered as a single injection, providing 56.3 mg bupivacaine and 30 μg dexamethasone.

AB-D solution was prepared to yield a aqueous bupivacaine concentration of 0.5%. The AB-D solution contained 5 mg bupivacaine and 10 μg dexamethasone. Three mL of AB-D was administered as a single injection to supply 15 mg aqueous bupivacaine and 30 μg dexamethasone.

The treatments were administered as an injection to the right or left wrist. Each subject received one injection of study drug in one wrist during treatment period 1, before crossing over to period 2, when he or she received the second treatment in the opposite wrist. The injection site was identified at the anatomic “snuffbox” made prominent by extension of the thumb. The extensor pollicis longus and brevis tendons were marked, and a point was identified over the extensor longus tendon opposite the base of the first metacarpal. A 21-gauge needle was directed proximally along the tendon as far as the dorsal radial tubercle, and a 2-mL suspension of 2.5% 120K EDLA or solution of 0.5% AB-D was injected subcutaneously. The needle was then withdrawn and redirected at a right angle across the snuffbox to a point just past the brevis tendon. A further 1-mL solution was then injected.

Efficacy measurements were onset and duration of analgesia/anesthesia, onset and duration of temperature perception block, incidence of analgesia/anesthesia and rate of complete blocks, and pharmacokinetics and pharmacodynamics measurements. Safety evaluations included pain on injection.

Analgesia/anesthesia block and temperature perception block testing was conducted at 0 hour to establish baseline sensory perception, and every 5 minutes up to 1 hour post-injection, or until onset of block. After 1 hour, analgesia/anesthesia block and temperature perception block testing continued every hour for 12 hours, or until the block offset. Thereafter, if the block had not offset, the analgesia/anesthesia block and temperature perception block testing were performed every hour while awake on the day the drug was administered, and thereafter, approximately every 4 to 6 hours until the block offset. Offset of block was defined as a return of normal sensation to all parts of the hand, and a return to baseline values for analgesia/anesthesia block and temperature perception block. Subjects returned to the site for follow-up efficacy and safety evaluations at 24, 48, and 72 hours post-injection, and for blood draws.

Onset And Duration Of Analgesia/Anesthesia (Pinprick)

In evaluating analgesia/anesthesia block, pinpricks were administered to a triangular area on the back of the hand, as shown in FIG. D1. Assessments were made by lightly tapping the skin with the dull end of a dental needle, using sufficient pressure to produce a sensation of sharpness (determined by first testing a nonaffected area). Each area was pricked 3 times and the subject was asked how many pinpricks, if any, were felt. Sensory block was rated as: Anesthesia subject felt 0/3 pinpricks; Analgesia=subject felt 2 or 3 of 3 pinpricks, perceived as touch or pressure; or, No block=subject felt 2 or 3 of 3 pinpricks, perceived as sharp.

If the subject reported feeling 2 pinpricks, of which one was perceived as touch or pressure and the other was perceived as sharp, the block was described as analgesia. The subject was considered to demonstrate analgesia if 2 out of 3 pinpricks were dull (that is, perceived as touch or pressure, rather than as being sharp). The subject was also considered to demonstrate anesthesia if only 1 pinprick was felt.

Table D1 shows the onset and duration of analgesia occurring in Area C only.

TABLE D1
Onset and Duration of Analgesia,^{a}Area C,
^{b}up to Day 7 (N = 6)

| | Sensory Block (Response to Pinprick) | | | | |
|---|---|---|---|---|---|
| | Subject | Onset | Offset | Duration | Total Duration |
| Treatment | (No.) | (Hour)^{c} | (Hour)^{d} | (Hour)^{e} | (Hour)^{e} |
| 2.5% 120K | 1 | 3:00 | 11:30 | 8:30 | 19:00 |
| EDLA | | | | | |
| | | 13:00 | 23:00 | 10:30 | |
| | 3 | 0:15 | 5:30 | 5:15 | 173:45 |
| | | 7:00 | 71:30 | 64:30 | |
| | | 76:00 | 180:00 | 104:00 | |
| | 5 | 6:00 | 38:00 | 32:00 | 32:00 |
| | | | | | Total Duration |
| | | | | | (Final Offset) |
| | | | | | (Hour) |
| 0.5% AB-D | 2 | 0:15 | 13:30 | 13:15 | 13:15 |
| | 4 | 0:15 | 19:00 | 18:45 | 18:45 |
| | 6^{f} | 1:00 | 1:30 | 0:30 | 15:30 |
| | | 3:00 | 13:30 | 10:30 | |
| | | 89:00 | 93:30 | 4:30 | |

^{a}None of the subjects reported anesthesia (0/5 pinpricks).
^{b}The TABLE shows periods of block occurring only in area C, the area on the back of the hand specified by the protocol as that designated for efficacy (pinprick) assessments.
^{c}Hours from injection, expressed as the first time the subject responded to mechanical stimulation (pinprick) felt as touch or pressure.
^{d}Midpoint between last time the subject responded to touch/pressure and the next measurement point with no analgesia.
^{e}Subjects with analgesia at day 7 are truncated to offset at 180 hours (168 + 12 hours).
^{f}The reonset of block in subject 6 at 4 days, was considered an anomaly, probably related to assessment technique.

Onset of anesthesia/analgesia was expressed as the first time when no sensation of pain from pinprick was recorded (for analgesia), or no sensation of touch or pressure was recorded (for anesthesia). None of the subjects experienced anesthesia, either with 120K EDLA or AB-D treatments. Onset of analgesia was later (15 minutes to 6 hours for 2.5% 120K EDLA versus 15 minutes to 1 hour for 0.5% AB-D). Offset (initial) for both treatments was variable (between 6 and 38 hours for 2.5% 120K EDLA and 2 and 19 hours for 0.5% AB-D).

Duration of analgesia/analgesia was expressed as the time between onset of anesthesia/analgesia and return to sensation of pain (when the block was rated analgesia) or touch or pressure (when the block was rated anesthesia). Duration of analgesia/anesthesia following 120K EDLA was differentiated according to assessment area. Most subjects experienced analgesia in area C. Some subjects also reported late-onset analgesia (beyond 7 days) in area D, i.e., the area of the thenar eminence and thumb (see FIG. D1). Reoccurrence of analgesia after the initial block offset occurred in a different part of the hand (the area of the thenar eminence, identified as area D).

As shown in Table D1, two subjects receiving 120K EDLA experienced more than one period of analgesia/anesthesia; therefore, the total duration is expressed as the aggregate of all periods of block. Duration of block in area C in 3 subjects receiving 2.5% 120K EDLA ranged between 19 hours and 7 days. One subject receiving 0.5% AB-D (subject 6) experienced 3 periods of block, with final offset occurring at 93:30 hours post-injection. The overall duration of analgesia ranged from 13 to 19 hours in this group.

The rate of complete blocks was defined as the percentage of blocks in which anesthesia was recorded within 3 hours of injection. A “partial block” was defined as a successful nerve block. A partial block was to include subjects who demonstrated analgesia, but not anesthesia, in response to pinprick.

Neither 120K EDLA nor AB-D resulted in anesthesia. Both 120K EDLA and AB-D resulted in analgesia, 3 in the 120K EDLA and 3 in the AB-D treatment groups. The absence of anesthesia in subjects receiving 3 mL of 0.5% aqueous bupivacaine was unexpected, given that this dose is usually associated with anesthesia. The absence of anesthesia was possibly explained by the anatomy of the superficial radial nerve, which is highly branched, and by the intersubject anatomical variability of this nerve.

Onset And Duration of Temperature Perception Block

Temperature perception (Somesthetic test) was assessed by touching each of the 4 assessment areas with a cold alcohol swab. The subject was instructed, “Tell me if you feel any change in temperature when I touch this swab to your skin.” Responses were recorded as “YES” (subject felt a change in temperature) or “NO” (subject had not felt a change in temperature).

Onset of temperature perception block occurred between 15 minutes and 6 hours post-injection with 2.5% 120K EDLA—slightly later than pain block, and with AB-D, occurred at the same time as onset of pain block (1 hour). Duration of temperature perception block was expressed as the time between onset temperature block and return to sensation of cold. Duration of temperature block followed the time course of blockade of pain perception but was usually shorter for both treatments, with offset usually occurring several hours prior to offset of sensory blockade.

Pharmacokinetic/Pharmacodynamic Measurements

Plasma bupivacaine concentrations were determined at each sampling time. Blood was obtained for determination of plasma bupivacaine at 15 minutes, 30 minutes, and at 1, 2, 3, 6, 9, and 12 hours post-injection, and at follow-up on day 7. Pharmacokinetic (PK) parameters were determined for plasma bupivacaine: (Cmax, Tmax, AUCt). The relation of plasma bupivacaine concentrations to the degree of response, measured as no block, analgesia, or anesthesia was observed.

Pharmacokinetic parameters were inestimable for all 3 subjects receiving 120K EDLA. For AB-D, the maximum exposure (Cmax) was 204, 249, and 182 ng/mL, and the total exposure (AUCt) was 356.8, 424.1, and 477.9 ng/mL·h for subjects 2, 4, and 6, respectively.

Among subjects receiving 2.5% 120K EDLA, plasma bupivacaine concentrations were undetectable or virtually undetectable in 2 subjects (subjects 1 and 3) (Table D2).

TABLE D2
Individual Plasma Bupivacaine Concentrations (ng/mL)
up to Day 7, Following Dosing With 2.5% 120K EDLA
and 0.5% AB-D (N = 6)

| | 2.5% 120K EDLA | | | 0.5% AB-D | | |
|---|---|---|---|---|---|---|
| Hour: minute | | | | | | |
| Post- | Concentration (ng/mL) | | | | | |
| injection | Subject | Subject | Subject | Subject | Subject | Subject |
| | 1 | 3 | 5 | 2 | 4 | 6 |
| 0:00 | 0.00 | 0.00 | 0.00 | 0.00 | 0.00 | 0.00 |
| 0:15 | 0.00 | —* | 5.85 | 204.00 | 249.00 | 182.00 |
| 0:30 | 0.00 | 0.00 | 9.18 | 176.00 | 187.00 | 144.00 |
| 1:00 | 0.00 | 0.00 | 8.51 | 113.00 | 110.00 | 82.30 |
| 2:00 | 0.00 | 0.00 | 8.14 | 47.80 | 47.80 | 52.20 |
| 3:00 | 0.00 | 0.00 | 7.06 | 29.70 | 34.80 | 37.60 |
| 6:00 | 0.00 | 0.00 | 6.86 | 12.30 | 15.60 | 17.50 |
| 9:00 | 0.00 | 0.00 | 8.84 | 7.27 | 10.50 | 12.10 |
| 12:00 | 0.00 | 0.00 | 9.20 | 0.00 | 9.00 | 8.57 |
| 24:00 | 0.00 | 24.00 | 30.80 | 0.00 | 0.00 | 6.04 |
| 48:00 | 0.00 | 0.00 | 14.90 | 0.00 | 0.00 | 0.00 |
| 72:00 | 0.00 | 0.00 | 0.00 | 0.00 | 0.00 | 0.00 |
| 1 week | 0.00 | 0.00 | 31.30 | 0.00 | 0.00 | 0.00 |

*Not recorded.

The maximum concentration with 2.5% 120K EDLA in any subject was 31.3 ng/mL, (subject 5) versus 249.0 ng/mL (subject 4) for aqueous bupivacaine. At the same time, the maximum bupivacaine dose delivered with 3 mL of 2.5% 120K EDLA was 56.25 mg versus 15 mg delivered as 3 mL of 0.5% aqueous bupivacaine.

Onset and offset of analgesia with 2.5% 120K EDLA were variable and showed no obvious relation to plasma concentrations, suggesting a local pharmacodynamic effect confined to the injection site (FIG. D2). By contrast, plasma bupivacaine concentrations in subjects 2, 4, and 6, who received aqueous bupivacaine (0.5% AB-D), behaved as predicted, displaying a prompt release and a relatively fast decline that coincided approximately with analgesic effect (FIG. D3).

### Example E

### Ascending Doses of EDLA in Superficial Radial Nerve Block

An open-label, comparative, dose-response study evaluated ascending doses of 120K EDLA administered as a superficial radial nerve block. Bilateral nerve blocks were administered to 3 subjects, using the lowest dose of 120K EDLA (3 mL of 0.312% suspension) in one wrist and the lowest dose of AB (3 mL of 0.25% solution) in the opposite wrist. Onset and duration of sensory block were assessed. If the duration of activity for 120K EDLA was less than 3 days, 3 more subjects were enrolled, and nerve blocks were administered to the second group using a higher dose (concentration and/or volume) of 120K EDLA and the higher dose of AB (0.5%). Each additional group of 3 subjects were enrolled and received a higher dose of 120K EDLA if administration of nerve blocks in the previous group had not demonstrated the desired 3- to 4-day duration of action. The maximum concentration of 120K EDLA was 2.5%; the maximum volume was 3 mL.

Efficacy measures included onset and duration of analgesia/anesthesia, onset and duration of temperature perception block, incidence of anesthesia and rate of unsuccessful blocks, and degree of numbness. Safety measures included pain on injection.

Onset and Duration of Analgesia/Anesthesia (Pinprick)

Analgesia/Anesthesia was evaluated using Response to Pinprick as a measure of efficacy. Sensory block was assessed by administering pinpricks to each of 4 designated areas on the back of each hand, as shown in FIG. D1, innervated by superficial radial nerve block. Assessments were made as set forth in Example D.

Onset of analgesia/anesthesia (sensory block) was categorized according to results of assessments performed at intervals up to 6 hours: ≦30 minutes, 30 minutes to 1 hour; 1 to 2 hours; 2 to 3 hours; 3 to 6 hours; and >6 hours. Duration of analgesia was categorized by results of assessments performed up to 6 hours following injection, and thereafter, approximately every 12 hours until the block offset. Assessment of onset and duration of analgesia/anesthesia was conducted in 4 assessment areas to determine individual variation in innervation. The most consistent incidence of block was observed in assessment area D, the thenar eminence and radial border of the thumb, versus areas A, B, and C. Table E1 provides data regarding onset and duration of block in area D, by treatment.

TABLE E1
Onset and Duration of Analgesia/Anesthesia, Assessment Area D

| Treatment Pairs | | | | | | | |
|---|---|---|---|---|---|---|---|
| 120K | | 120K | | 120K | | Combined | |
| EDLA | AB | EDLA | AB | EDLA | AB | 120K | |
| 0.312% | 0.25% | 0.625% | 0.5% | 1.25% | 0.5% | EDLA | AB |

| | | | |
|---|---|---|---|
| (N = 3) | (N = 3) | (N = 6) | (N = 12) |

Number (%) of Subjects With Analgesia/Anesthesia*

| | | | | | | | |
|---|---|---|---|---|---|---|---|
| 1(33) | 3(100) | 2(67) | 3(100) | 5(83) | 6(100) | 8(67) | 12(100) |

Onset, Number (%) of Subjects**

| | | | | | | | | |
|---|---|---|---|---|---|---|---|---|
| ≦30 | 0(0) | 1(33) | 0(0) | 2(67) | 0(0) | 4(67) | 0(0) | 7(58) |
| min | | | | | | | | |
| >30 | 0(0) | 2(67) | 0(0) | 1(33) | 1(17) | 2(33) | 1(8) | 5(42) |
| min-1 h | | | | | | | | |
| >1-≦2 h | 0(0) | 0(0) | 1(33) | 0(0) | 2(33) | 0(0) | 3(25) | 0(0) |
| >2-≦3 h | 0(0) | 0(0) | 0(0) | 0(0) | 0(0) | 0(0) | 0(0) | 0(0) |
| >3-≦6 h | 0(0) | 0(0) | 1(33) | 0(0) | 0(0) | 0(0) | 1(8) | 0(0) |
| >6h | 1(33) | 0(0) | 0(0) | 0(0) | 2(33) | 0(0) | 3(25) | 0(0) |

Duration (Days)

| | | | | | | | | |
|---|---|---|---|---|---|---|---|---|
| Mean | 0.33 | 0.47 ± 0.22 | 0.76 ± 0.74 | 0.50 ± 0.34 | 5.38 ± 1.86 | 0.54 ± 0.15 | 3.60 ± 1.42 | 0.52 ± 0.11 |
| Range | 0.33-0.33 | 0.04-0.69 | 0.02-1.50 | 0.16-1.19 | 0.02-9.18 | 0.16-1.18 | 0.02-9.18 | 0.04-1.19 |

*Anesthesia = 0/3 pinpricks felt. Analgesia = 2/3 pinpricks felt as touch or pressure.
**The denominator for percent of subjects is the total number of subjects dosed, rather than the total number of subjects with analgesia/anesthesia.

As can be seen from the results provided in Table E1 the onset of analgesia/aesthesia was later and the duration longer for 120K EDLA compared to AB in area D. Onset ranged between 1 and 6 hours, and the mean duration was 3.60±1.42 days. The range was 0.02 to 9.18 days, which was considerably longer than the duration observed with 0.5% AB (mean, 0.52 days; range, 0.04-1.19 days).

In area D, the 1.25% concentration of 120K EDLA resulted in some level of block in ⅚ (83%) of subjects, compared to ⅔ and ⅓ in for the 0.625% and the 0.312% concentrations, respectively. Three subjects (subjects 008, 010, and 011) who received 120K EDLA had sensory block recurring to days 15, 17, and 50, post-injection, respectively.

The most definitive block was observed for the highest concentration of EDLA (1.25%) vs the lower concentrations. To facilitate evaluation of effect differences in assessment areas, onset and duration of block are shown for 1.25% EDLA, by assessment area, in Table E2.

TABLE E2
Onset and Duration of Analgesia/Anesthesia for 1.25% 120K EDLA, By Assessment Area

| | Area A | | Area B | | Area C | | Area D | |
|---|---|---|---|---|---|---|---|---|
| | 120K EDLA | AB | 120K EDLA | AB | 120K EDLA | AB | 120K EDLA | AB |
| | (N = 6) | | (N = 6) | | (N = 6) | | (N = 6) | |
| Number (%) of | | | | | | | | |
| Subjects With | | | | | | | | |
| Analgesia/Anesthesia | | | | | | | | |
| | 2 (33) | 5 (83) | 4 (67) | 6 (100) | 4 (67) | 6 (100) | 5 (83) | 6 (100) |
| Onset, Number | | | | | | | | |
| (%) of Subjects* | | | | | | | | |
| ≦30 min | 0 (0) | 1 (17) | 0 (0) | 2 (33) | 0 (0) | 4 (67) | 0 (0) | 4 (67) |
| >30 min-1 h | 0 (8) | 3 (50) | 1 (17) | 3 (50) | 0 (0) | 2 (33) | 1 (17) | 2 (33) |
| >1-≦2 h | 2 (33) | 1 (17) | 2 (33) | 0 (0) | 2 (33) | 0 (0) | 2 (33) | 0 (0) |
| >2-≦3 h | 0 (0) | 0 (0) | 0 (0) | 1 (17) | 0 (0) | 0 (0) | 0 (0) | 0 (0) |
| >3-≦6 h | 0 (0) | 0 (0) | 1 (17) | 0 (0) | 2 (33) | 0 (0) | 0 (0) | 0 (0) |
| >6 h | 0 (0) | 0 (0) | 0 (0) | 0 (0) | 0 (0) | 0 (0) | 2 (33) | 0 (0) |
| Duration (Days) | | | | | | | | |
| Mean (±SE) | 0.55 ± 0.53 | 0.35 ± 0.14 | 0.36 ± 0.24 | 0.45 ± 0.11 | 0.78 ± 0.34 | 0.57 ± 0.16 | 5.38 ± 1.86 | 0.54 ± 0.15 |
| Range | 0.02-1.08 | 0.02-0.69 | 0.01-1.08 | 0.02-0.69 | 0.20-1.59 | 0.08-1.14 | 0.02-9.18 | 0.16-1.18 |

The denominator for percent of subjects is the total number of subjects dosed, rather than the total number of subjects with analgesia/anesthesia.

Onset of sensory block with 1.25% 120K EDLA was similarly variable in the 4 assessment areas, but duration was notably longer in area D (5.38±1.86 days vs 0.55±0.53, 0.36±0.24, and 0.78±0.34 days in areas A, B, and C, respectively). One subject who received 1.25% 120K EDLA experienced reonset of block that continued to day 50 post-injection.

With 0.5% AB treatment, onset generally occurred between 15 minutes to1 hour; and duration of block was comparable (approximately 12 hours) across assessment areas B, C, and D. Duration of block in area A was somewhat shorter (0.35±0.14, ie, approximately 10 hours).

FIG. E1 shows mean pinprick scores for each treatment up to 50 days, which was the maximum duration of block exhibited by any subject. The 1.25% concentration of 120K EDLA demonstrated the most definitive block and held the most interest as a potentially therapeutic dose. Table E3 summarizes the mean pinprick scores for 1.25% 120K EDLA compared to 0.5% AB up to day 7, by each assessment area and for combined areas.

TABLE E3
Period of Block (Analgesia/Anesthesia*) in Response to Pinprick, for 120K EDLA 1.25%
and AB 0.5%

[symbol]

[symbol]

[symbol]

[symbol]

*Period of block is shown in enclosed areas.
**Anesthesia (0) = none of 3 pinpricks detected; analgesia (1) = 2 of 3 pinpricks detected as touch or pressure; no block (2) = 2 or more pinpricks detected as sharp.

The extended duration of block occurred with 120K EDLA in area C and was accounted for by the extended duration of analgesia observed with 1.25% 120K EDLA in subjects 8 and 10 (up to days 15 and 17, respectively). As shown in FIG. E1, the spikes in areas A, C, and D represent the reonset of block experienced by subject 11 at 41 days, which continued in area D without resolution to day 50. Generally, the block set up later with 120K EDLA compared to AB, although onset was earlier (30 minutes) in area D. With 120K EDLA, duration was similar to AB in areas A and B and was longer in areas C and D, where duration extended to day 2 and day 7, respectively. With 0.5% AB, the block occurred between 30 minutes and 3 hours and offset in all areas by the end of day 1.

Onset and Duration of Temperature Perception Block

Temperature perception was assessed as set forth in Example D. Onset of temperature block was categorized according to results of assessments performed at intervals up to 6 hours: ≦30 minutes, 30 minutes to 1 hour, 1 to 2, 2 to 3, 3 to 6 hours, and >6 hours. Duration of temperature block was categorized by results of assessments performed up to 6 hours following injection, and thereafter, approximately every 12 hours until the block offset. Assessment of onset and duration of temperature block was conducted in 4 assessment areas to determine the dispersibility of the microsphere preparation, and the disposition of local anesthetic.

EDLA resulted in the most definitive temperature block in assessment area D, the thenar eminence and radial border of the thumb, compared to areas A, B, and C. Onset and duration of block in area D are shown, by treatment, in Table E4.

TABLE E4
Onset and Duration of Temperature Perception Block, Assessment Area D, by Dose Pairs and Overall

| | Treatment Pairs | | | | | | Combined | |
|---|---|---|---|---|---|---|---|---|
| | 120K EDLA | AB | 120K EDLA | AB | 120K EDLA | AB | Treatments | |
| | 0.312% | 0.25% | 0.625% | 0.5% | 1.25% | 0.5% | 120K EDLA | AB |
| | (N = 3) | | (N = 3) | | (N = 6) | | (N = 12) | |
| Number (%) of | | | | | | | | |
| Subjects With | | | | | | | | |
| Temperature | | | | | | | | |
| Perception Block | | | | | | | | |
| | 0 (0) | 2 (67) | 2 (67) | 3 (100) | 5 (83) | 6 (100) | 7 (58) | 11 (92) |
| Onset, Number | | | | | | | | |
| (%) of Subjects* | | | | | | | | |
| ≦30 min | 0 (0) | 1 (33) | 0 (0) | 2 (67) | 0 (0) | 3 (50) | 0 (0) | 6 (50) |
| >30 min-1 h | 0 (0) | 1 (33) | 0 (0) | 1 (33) | 1 (17) | 3 (50) | 1 (8) | 5 (42) |
| >1-≦2 h | 0 (0) | 0 (0) | 0 (0) | 0 (0) | 2 (33) | 0 (0) | 2 (17) | 0 (0) |
| >2-≦3 h | 0 (0) | 0 (0) | 0 (0) | 0 (0) | 0 (0) | 0 (0) | 0 (0) | 0 (0) |
| >3-≦6 h | 0 (0) | 0 (0) | 1 (33) | 0 (0) | 1 (17) | 0 (0) | 2 (17) | 0 (0) |
| >6 h | 0 (0) | 0 (0) | 1 (33) | 0 (0) | 1 (17) | 0 (0) | 2 (17) | 0 (0) |
| Duration (Days) | | | | | | | | |
| Mean | — | 0.35 ± 0.34 | 0.65 ± 0.32 | 0.12 ± 0.08 | 2.08 ± 1.56 | 0.45 ± 0.17 | 1.67 ± 1.11 | 0.34 ± 0.11 |
| Range | — | 0.01-0.69 | 0.33-0.97 | 0.01-0.28 | 0.02-8.16 | 0.01-1.18 | 0.02-8.16 | 0.01-1.18 |

*The denominator for percent of subjects is the total number of subjects dosed, rather than the total number of subjects with analgesia/anesthesia.

Examination of temperature block in assessment Area D revealed the 1.25% concentration of 120K EDLA demonstrated the most consistent effect compared to lower concentrations (Table E6). Onset ranged between 1 and 6 hours, and the mean duration was 2.08±1.56 days. The range was 0.02 to 8.16 days, which was longer than the duration observed in the same group with 0.5% AB (mean, 0.45±0.17 days; range, 0.01 to 1.18 days). The lowest (0.132%) concentration had no effect on temperature perception, and the 0.625% concentration had only a minor effect. AB 0.5%, by comparison, resulted in temperature block within 1 hour; and the block lasted between approximately 1 hour and 1 day.

As with pinprick evaluations, the most consistent temperature perception block was observed with the 1.25% concentration of 120K EDLA. Table E5 summarizes the onset and duration of temperature block for the 1.25% concentration, for the 4 assessment areas.

TABLE E5
Onset and Duration of Temperature Perception Block* for 1.25% 120K EDLA, by Assessment Area

| | Area A | | Area B | | Area C | | Area D | |
|---|---|---|---|---|---|---|---|---|
| | 120K EDLA | AB | 120K EDLA | AB | 120K EDLA | AB | 120K EDLA | AB |
| | (N = 6) | | (N = 6) | | (N = 6) | | (N = 6) | |
| Number (%) of | | | | | | | | |
| Subjects With | | | | | | | | |
| Temperature | | | | | | | | |
| Perception Block* | | | | | | | | |
| | 2 (33) | 5 (83) | 3 (50) | 6 (100) | 5 (83) | 6 (100) | 5 (83) | 6 (100) |
| Onset, Number | | | | | | | | |
| (%) of Subjects | | | | | | | | |
| ≦30 min | 0 (0) | 0 (0) | 0 (0) | 3 (50) | 0 (0) | 3 (50) | 0 (0) | 3 (50) |
| >30 min-1 h | 1 (17) | 2 (33) | 0 (0) | 3 (50) | 0 (0) | 3 (50) | 1 (8) | 3 (50) |
| >1-≦2 h | 1 (17) | 2 (33) | 2 (17) | 0 (0) | 1 (8) | 0 (0) | 2 (17) | 0 (0) |
| >2-≦3 h | 0 (0) | 0 (0) | 0 (0) | 0 (0) | 0 (0) | 0 (0) | 0 (0) | 0 (0) |
| >3-≦6 h | 0 (0) | 0 (0) | 1 (8) | 0 (0) | 0 (0) | 0 (0) | 1 (8) | 0 (0) |
| >6 h | 0 (0) | 1 (17) | 0 (0) | 0 (0) | 4 (33) | 0 (0) | 1 (8) | 0 (0) |
| Duration (Days) | | | | | | | | |
| Mean(±SE) | 0.57 ± 0.55 | 0.13 ± 0.06 | 0.49 ± 0.31 | 0.18 ± 0.10 | 0.65 ± 0.25 | 0.53 ± 0.10 | 2.08 ± 1.56 | 0.45 ± 0.17 |
| Range | 0.02-1.12 | 0.02-0.31 | 0.02-1.08 | 0.01-0.65 | 0.18-1.42 | 0.04-0.69 | 0.02-8.16 | 0.02-1.18 |

*Successful block = Subject does not detect a change in temperature. Failed block = Subject detects a change in temperature.

The 1.25% concentration of 120K EDLA resulted in variable onset of temperature block, ranging from approximately 1 hour to >6 hours, across all assessment areas. The upper end of the duration range was notably longer in area D (8.16 days) compared to areas A (1.12 days), B (1.08 days), and C (1.42 days). For 0.5% AB, between-area assessments showed little differences in activity, although onset tended to be later and duration shorter in area A.

Onset of temperature perception block with 120K EDLA was not appreciably different from onset of block of pain perception, but duration was shorter (2.08±1.56 vs 5.38±1.86, for temperature and pain block, respectively).

TABLE E6
Period of Temperature Perception Block* for 120K EDLA 1.25% and AB 0.5%

[symbol]

[symbol]

[symbol]

[symbol]

*Period of block is shown in enclosed areas.
**Score 0 = Subject does not detect a change in temperature. Score 1 = Subject detects a change in temperature.

The principal differences between treatments were in the extended duration of block occurring with 120K EDLA, and specifically, with 1.25% 120K EDLA in areas C and D. In area C, these differences were largely accounted for by the extended effects of 1.25% 120K EDLA in two subjects. With 120K EDLA treatment, blockade of temperature perception occurred between 1 and 3 hours and offset by day 2 (1 day later than offset of sensory block), except in area D, where temperature block continued beyond day 7. Temperature perception block with AB treatment occurred within 30 minutes and had an offset by day 2, which was later than offset of sensory block (day 1).

Incidence of Analgesia/Anesthesia

Table E7 shows the incidence of anesthesia, analgesia, and unsuccessful block in area D, by treatment.

TABLE E7
Incidence (Number [%] of Subjects) of Anesthesia, Analgesia, and Unsuccessful Sensory
Block in Assessment Area D, by Treatment

| | | | | | | | Combined | |
|---|---|---|---|---|---|---|---|---|
| | Treatment Pairs | | | | | | Treatments | |
| | 120K EDLA | AB | 120K EDLA | AB | 120K EDLA | AB | | |
| | 0.312% | 0.25% | 0.625% | 0.5% | 1.25% | 0.5% | 120K EDLA | AB |
| | (N = 3) | | (N = 3) | | (N = 6) | | (N = 12) | |
| Test Result | Number (%) of Subjects | | | | | | | |
| Anesthesia* | 0 (0) | 0 (0) | 1 (33) | 2 (67) | 2 (33) | 2 (33) | 3 (25) | 4 (33) |
| Analgesia | 1 (33) | 3 (100) | 1 (33) | 1 (33) | 3 (50) | 4 (67) | 5 (42) | 8 (67) |
| Analgesia/Anesthesia* | 1 (33) | 3 (100) | 2 (67) | 3 (100) | 5 (83) | 6 (100) | 8 (67) | 12 (100) |
| No block† | 2 (67) | 0 (0) | 1 (33) | 0 (0) | 1 (17) | 0 (0) | 4 (33) | 0 (0) |

*Anesthesia = 0/3 pinpricks felt.
**Analgesia = 2/3 pinpricks felt as touch or pressure.
†No block = ≧2 pinpricks felt as sharp.
§Percent of subjects shown in the table refers to overall incidence across all 4 assessment areas.

In area D, anesthesia was reported in 3 (25%) subjects receiving 120K EDLA and in 4 (33%) of subjects receiving 0.5% AB. Anesthesia occurred in 0/3 subjects receiving the lowest concentration (0.312%) 120K EDLA, ⅓ subjects receiving 0.625% 120K EDLA, and in ⅔ subjects receiving 1.25% 120K EDLA. AB resulted in anesthesia only with the 0.5% concentration (3 mL).

Analgesia/anesthesia occurred at 8/12 (67%) of 120K EDLA injection sites, occurred at least once with all doses, and occurred more consistently with high versus low 120K EDLA dose/concentrations (83% incidence with 1.25%, 67% with 0.625%, and 33% with 0.312% 120K EDLA). Analgesia/anesthesia was observed at 100% of sites treated with AB (0.25% and 0.5%). Four subjects (33%) reported no block with 120K EDLA treatment, 2 receiving 0.312%, 1 receiving 0.625% 120K EDLA, and 1 receiving 1.25% 120K EDLA.

Table E8 shows the overall incidence (combined 120K EDLA and combined AB) of anesthesia, analgesia, and unsuccessful sensory block, by assessment area.

TABLE E8
Incidence (Number [%] of Subjects) of Anesthesia, Analgesia, and Unsuccessful Sensory
Block for Combined EDLA and Combined AB Treatments, by Assessment Area

| | Assessment Areas | | | | | | | | | |
|---|---|---|---|---|---|---|---|---|---|---|
| | Area A | | Area B | | Area C | | Area D | | Combined Areas | |
| | 120K EDLA | AB | 120K EDLA | AB | 120K EDLA | AB | 120K EDLA | AB | 120K EDLA | AB |
| | (N = 12) | | (N = 12) | | (N = 12) | | (N = 12) | | (N = 48) | |
| Test Result | Number (%) of Subjects | | | | | | | | | |
| Anesthesia** | 1 (8) | 2 (17) | 3 (25) | 2 (17) | 3 (25) | 3 (25) | 3 (25) | 4 (33) | 10 (21) | 11 (23) |
| Analgesia | 3 (25) | 9 (75) | 3 (25) | 10 (83) | 2 (17) | 8 (67) | 5 (42) | 8 (67) | 13 (27) | 35 (73) |
| Analgesia/ | 4 (33) | 11 (92) | 6 (50) | 12 (100) | 5 (42) | 11 (92) | 8 (67) | 12 (100) | 23 (50) | 46 (96) |
| Anesthesia* | | | | | | | | | | |
| No block† | 8 (67) | 1 (8) | 6 (50) | 0 (0) | 7 (58) | 1 (8) | 4 (33) | 0 (0) | 25 (52) | 2 (4) |

*Analgesia = 2/3 pinpricks (felt as touch/pressure).
**Anesthesia = 0/3.
†No block = ≧3.

Of the 4 designated assessment areas on the injected hand, area D, proximal to the thenar eminence and lateral border of the thumb, exhibited the most consistent block with 120K EDLA treatment, with 67% of subjects demonstrating at least analgesia, and 25% demonstrating anesthesia. Area A exhibited the least responsiveness to 120K EDLA treatment, with 33% demonstrating analgesia and 8% demonstrating anesthesia. This result was thought to be related to the known variability in the anatomical structure of the superficial radial nerve, rather than to any intrinsic variability in the behavior of the microspheres.

Aqueous bupivacaine 0.5% resulted in a relatively consistent block, with only small differences noted between assessment areas with respect to analgesia/anesthesia. However, anesthesia alone was observed more frequently in area D compared to other assessment areas, an observation that agreed with the relatively greater responsiveness in area D seen with 120K EDLA treatment.

Degree of Numbness

In evaluating Level of Numbness, the subject or investigator assessed level of numbness by touching each of the 4 assessment areas on each hand. Subjects were asked to rate the degree of numbness based on an 11-point scale, on which 0 was equal to “not numb at all,” and 10 was equal to “totally numb.” Level of numbness scores tended to reflect the rapid onset and shorter duration of AB, and the more gradual onset and longer duration of 120K EDLA. Table E9 summarizes the mean level of numbness scores to day 7, by time point.

TABLE E9
Period of Numbness* (Any Level) for 120K EDLA 1.25% and AB 0.5%

[symbol]

[symbol]

[symbol]

[symbol]

*Numbness was rated on an 11-point scale, on which 0 = not numb at all and 10 = totally numb.

As shown in Table E9, some level of numbness occurred for AB at each time point between 30 minutes and day 2, while for 120K EDLA, some level of numbness occurred between 30 minutes and >7 days. Level of numbness scores tended to be higher at all times for AB compared to 120K EDLA, although the magnitude of the range was similar for 120K EDLA (0.33 to 4.25) and AB (1.25 to 8.17). Level of numbness scores over time with 1.25% 120K EDLA were lower compared to AB at all time points up to the latter part of day 1, when AB scores declined and 120K EDLA scores were highest. Level of numbness scores were highest at 1 hour post-injection for AB, and at day 1 PM assessments for 120K EDLA, continuing with 120K EDLA treatment to demonstrate some level of numbness up to day 7. Consistent with results of pinprick and temperature perception assessments, mean level of numbness scores indicated that most 120K EDLA activity occurred in areas C and D, with an earlier onset and notable extended duration of effect in area D.

Conclusions

In area D, 120K EDLA, at the highest concentration (1.25%) resulted in anesthesia/analgesia in 83% of the subjects and in anesthesia or analgesia in two thirds of the subjects. The 1.25% dose/concentration of 120K EDLA demonstrated the most consistent analgesic effect compared to lower EDLA concentrations, according to all efficacy measures. Onset was between 1 and 6 hours and duration was at least 5 days. Assessment area D demonstrated a comparatively greater response to 120K EDLA and AB compared to other assessment areas. This result was thought to be related to the known variability in the anatomical structure of the superficial radial nerve, rather than to any intrinsic variability in the behavior of the microspheres.

### Example F

### Evaluating the Safety and Sensory Blockade Characteristics of 40K EDLA and 40K IDLA When Administered to the Superficial Radial Nerve

An open-label, comparative, ascending dose-response study compared 40K EDLA, 40K IDLA, and aqueous bupivacaine (AB). This study was conducted in two parts. In Part 1, bilateral superficial radial nerve blocks were administered to successive groups of three (3) subjects until a dose of 40K EDLA demonstrated a sensory block for a duration of approximately three (3) to five (5) days. Aqueous Bupivacaine (0.5%) was used as a reference treatment (AB). Following assessment of duration of sensory block (if less than three [3] days), the next group of three (3) subjects was enrolled and was administered bilateral nerve blocks using a higher dose (concentration) of 40K EDLA and a constant dose of the reference treatment (AB). Additional groups of three (3) subjects were enrolled following assessment and resolution of the nerve blocks in the previous group. Adjustments in the concentration of 40K EDLA for each subsequent group of three (3) subjects were determined. The concentrations of 40K EDLA were 0.624%, 1.25%, and 2.5%. The volume per injection was 3 mL. The same volume (3 mL) was used for injection of 40K EDLA, 40K IDLA and AB.

In Part 2, six (6) subjects received a superficial radial nerve block on one wrist only (the wrist of the non-dominant hand). Three (3) subjects were administered the selected dose of 40K EDLA that was identified in Part 1 as 1.25%. The other three (3) subjects were administered 40K IDLA at the equivalent dose. Blood samples were taken for plasma bupivacaine and dexamethasone levels at 0, 3 and 6 hours post-injection and daily thereafter until the block resolved. In addition, changes in the amplitude and velocity of radial nerve conduction were assessed.

Efficacy evaluations included onset and duration of analgesia/anesthesia, onset and duration of temperature perception block, incidence of analgesia/anesthesia and rate of unsuccessful block, degree of numbness, mechanical touch detection threshold, and pharmacokinetic measures. To determine the extent and timing of nerve recovery after extended blockade, nerve conduction studies were performed to measure the latency and amplitude of the radial sensory response.

The superficial radial nerve innervates the area of the hand from the radial border near the thumb to the middle of the back of the hand. This area was divided into four (4) test areas as shown in FIG. D1, which were designated A, B, C, and D. Assessment of efficacy was conducted in these four (4) assessment areas. The pattern of local anesthetic effects in Areas A through D varied within and across subjects, presumably due to variation in the pattern of distal radial nerve innervation. Across doses of 40K EDLA, the most consistent incidence and duration of block was observed in assessment area C versus areas A, B, and D. Therefore, data for area C are presented for all efficacy measures (primary and secondary). Onset And Duration Of Analgesia/Anesthesia Pinprick testing was performed in each of the four designated areas (A, B, C, and D) the area that appeared to demonstrate the most pronounced change from baseline pinprick results was marked with a circle about the size of a dime. All subsequent pinprick tests were performed within each of these four (4) circles and within each of the four (4) designated areas. If an area had not demonstrated any sensory block, the area was tested with pinpricks but without drawing a circle. Each circle was “pricked” approximately three (3) times with the dull end of a needle and the subject was asked to state how many of the pinpricks were felt. If the subject felt some of the pinpricks, the subject was asked how many were felt as sharp or as touch/pressure. The number of pinpricks felt as sharp or as touch/pressure (rated as analgesia) was recorded for each area as 0=subject did not feel any pinpricks (rated as anesthesia); 1=subject felt 2 (rated as an unsuccessful block) or 3 (out of 3) pinpricks as touch or pressure; or, 2=subject felt 2 or 3 (out of 3) pinpricks as sharp. If only two (2) pinpricks were felt and one (1) was felt as touch or pressure and the other was felt as sharp, or if only one (1) pinprick was felt, the level of “1” (touch/pressure) was assigned.

Onset and duration of analgesia with or without anesthesia were assessed by the investigator and by the subject. Onset of analgesia with or without anesthesia was defined as the first time at which pinprick testing on the top of the hand demonstrated analgesia (touch/pressure) or anesthesia (no pinpricks felt). Pinprick testing for onset of sensory block was performed at Baseline, and post-injection at approximately hours 0.5, 1, 2, 3 and 6.

Duration of analgesia with or without anesthesia was defined as the time between onset of analgesia with or without anesthesia and time when there was a return of sensation of sharpness to pinpricks in a given area. Subjects returned to the study site approximately every 24 hours for pinprick testing by the evaluator until the offset of sensory block was determined. Subjects were also instructed on how to perform the pinprick assessments at home.

The first self-evaluation was performed at 12 hours post-injection and thereafter approximately every 12 hours following the investigator's assessment at each daily return visit. Self-assessments continued once in the morning and again in the evening for 14 consecutive days post-injection, regardless of offset. All subjects returned for evaluations on Days 7 and 14, regardless of offset.

Onset of Analgesia/Anesthesia: 40K EDLA versus AB

To illustrate the effect of differences in assessment areas in Study Part 1, onset and duration of block are shown-for 1.25% 40K EDLA, by assessment area, in Table F1.

TABLE F1
Onset and Duration of Analgesia/Anesthesia for 1.25% 40K EDLA, by Assessment Area^{a}
Study Part 1

| | Area A | | Area B | | Area C | | Area D | |
|---|---|---|---|---|---|---|---|---|
| | Treatment Pair | | Treatment Pair | | Treatment Pair | | Treatment Pair | |
| | 40K EDLA | AB | 40K EDLA | AB | 40K EDLA | AB | 40K EDLA | AB |
| | 1.25% | 0.5% | 1.25% | 0.5% | 1.25% | 0.5% | 1.25% | 0.5% |
| | N = 3 | | N = 3 | | N = 3 | | N = 3 | |
| Number (%) of | | | | | | | | |
| Subjects With | | | | | | | | |
| Analgesia/Anesthesia | | | | | | | | |
| (No., [%]) | | | | | | | | |
| | 2 (67%) | 3 (100%) | 3 (100%) | 2 (67%) | 3 (100%) | 3 (100%) | 3 (100%) | 3 (100%) |
| Time to Onset of | | | | | | | | |
| Analgesia/Anesthesia | | | | | | | | |
| (No., [%]) | | | | | | | | |
| ≦30 min | 0 | 3 (100%) | 0 | 1 (33%) | 1 (33%) | 3 (100%) | 0 | 1 (33%) |
| >30 min-1 hr | 0 | 0 | 0 | 0 | 0 | 0 | 0 | 0 |
| >1-2 hrs | 1 (33%) | 0 | 1 (33%) | 0 | 0 | 0 | 1 (33%) | 1 (33%) |
| >2-3 hrs | 0 | 0 | 0 | 1 (33%) | 0 | 0 | 0 | 1 (33%) |
| >3-6 hrs | 0 | 0 | 0 | 0 | 1 (33%) | 0 | 1 (33%) | 0 |
| >6 hrs | 1 (33%) | 0 | 2 (67%) | 0 | 1 (33%) | 0 | 1 (33%) | 0 |
| Duration (Days) | | | | | | | | |
| Mean (±SE) | 0.16 (0.10) | 0.05 (0.02) | 0.37 (0.17) | 0.38 (0.35) | 1.53 (0.82) | 0.33 (0.20) | 0.98 (0.97) | 0.04 (0.02) |
| Range | 0.05-0.26 | 0.01-0.09 | 0.14-0.70 | 0.03-0.73 | 0.01-2.82 | 0.09-0.73 | 0.00-2.93 | 0.02-0.08 |

^{a}Analgesia = subjects who felt 2 or 3 of 3 pinpricks as touch/pressure. Anesthesia = subjects who did not feel any of 3 pinpricks.

Onset of analgesia/anesthesia with 1.25% 40K EDLA was slightly faster assessment areas C and D compared to areas A and B. Areas C and D had onset ocurring within six (6) hours in 67% of the blocks whereas areas A and B onset of 33% of the blocks ocurring within six (6) hours. With 0.5% AB treatment, onset generally occurred within six (3) hours in all areas. Areas A and C had 100% of the blocks ocurring within less than 30 minutes. Across 40K EDLA doses, onset of block occurred within six (6) hours in 78% of the blocks in area C compared to within 30 minutes for 100% of blocks with AB. Table F2 summarizes these results.

TABLE F2
Onset and Duration of Analgesia/Anesthesia^{a} in Area C
Study Part 1

| | Treatment Pair | | Treatment Pair | | Treatment Pair | | Study Part 2 | |
|---|---|---|---|---|---|---|---|---|
| | 40K EDLA | AB | 40K EDLA | AB | 40K EDLA | AB | 40K EDLA | 40K IDLA |
| | 0.625% | 0.5% | 1.25% | 0.5% | 2.5% | 0.5% | 1.25% | 1.25% |
| | N = 3 | | N = 3 | | N = 3 | | N = 3 | N = 3 |
| Time to Onset of | | | | | | | | |
| Analgesia/Anesthesia | | | | | | | | |
| (No., [%]) | | | | | | | | |
| ≦30 min | 0 | 3 (100%) | 1 (33%) | 3 (100%) | 1 (33%) | 3 (100%) | 2 (67%) | 2 (67%) |
| >30 min-1 hr | 1 (33%) | 0 | 0 | 0 | 0 | 0 | 0 | 0 |
| >1-2 hrs | 1 (33%) | 0 | 0 | 0 | 1 (33%) | 0 | 0 | 0 |
| >2-3 hrs | 0 | 0 | 0 | 0 | 0 | 0 | 0 | 0 |
| >3-6 hrs | 0 | 0 | 1 (33%) | 0 | 1 (33%) | 0 | 1 (33%) | 0 |
| >6 hrs | 1 (33%) | 0 | 1 (33%) | 0 | 0 | 0 | 0 | 1 (33%) |
| Duration (days) | | | | | | | | |
| Mean | 1.65 | 0.53 | 1.53 | 0.33 | 2.23 | 1.00 | 0.84 | 1.09 |
| (SE) | 1.28 | 0.36 | 0.82 | 0.20 | 1.22 | 0.10 | 0.82 | 0.99 |
| Range | 0.02-4.17 | 0.01-1.22 | 0.01-2.82 | 0.09-0.73 | 0.02-4.22 | 0.90-1.20 | 0.01-2.48 | 0.04-3.07 |

^{a}Analgesia = subjects felt 2 or 3 of 3 pinpricks as touch/pressure. Anesthesia = subjects did not feel any of 3 pinpricks.

Onset of block was slightly slower for lower concentrations of 40K EDLA compared to higher concentrations, with onset occurring within 6 hours in 67% of the blocks for the 0.625% and 1.25% concentrations and in 100% of the blocks for the 2.5% concentration, as shown in Table F2. FIG. F1 shows the percent of subjects demonstrating onset of analgesia.anesthesia within 6 hours for 1.25% 40 K EDLA, in comparison with aqueous bupivacaine.

Duration of analgesia/anesthesia was defined as the time between the first onset of analgesia and the time when there was a return of a sensation of sharpness in response to pinprick testing (i.e., loss of analgesia). were statistically significant (p<0.05): a decrease in radial pulse for the 15 mL bilateral AB 0.5% treatment and a decrease in temperature for the unilateral 15 mL 40K EDLA 2.5% treatment, the bilateral 15 mL 40K EDLA 2.5% treatment, and the bilateral 15 mL AB 0.25% treatment. None of the mean changes in vital signs results from baseline to final visit were considered clinically meaningful.

Clinically Notable Vital Sign Abnormalities

Table J-19 lists clinically notable vital sign abnormalities by subject and parameter, along with all other values during the study for that vital sign and parameter and other relevant vital sign parameters at selected time points.

TABLE J-19
Clinically Notable Vital Sign Abnormalities
Safety Population (N = 28)

| | | | SBP | DBP | HR | RR |
|---|---|---|---|---|---|---|
| Treatment Group | Subject | Visit | (mmHg)^{a} | (mmHg)^{a} | (bpm)^{a} | (breaths/min)^{a} |
| 15 mL 40K | 213 | Screening | 112 | 64 | 60 | 16 |
| EDLA 1.25% + | | | | | | |
| 15 mL 40K | | | | | | |
| EDLA 2.5% | | | | | | |
| | | Injection day | 104 | 41^{b} | 48 | 16 |
| | | 24 h post-inj. | 119 | 53 | 69 | 16 |
| | | 48 h post-inj. | 107 | 39 | 55 | 14 |
| | | 72 h post-inj. | 103 | 44 | 55 | 16 |
| | | 96 h post-inj. | 111 | 69 | 61 | 14 |
| 15 mL 40K | 107 | Screening | 110 | 80 | 76 | 14 |
| EDLA 2.5% | | | | | | |
| bilateral | | | | | | |
| | | Injection day | 123 | 75 | 49^{c} | 16 |
| | | 24 h post-inj. | 122 | 78 | 69 | 12 |
| 15 mL AB 0.5% | 106 | Screening | 118 | 70 | 76 | 14 |
| bilateral | | | | | | |
| | | Injection day | 136 | 81 | 68 | 18 |
| | | 24 h post-inj. | 134 | 86 | 68 | 10 |
| | | 48 h post-inj. | 136 | 87 | 68 | 16 |
| 15 mL 40K | 104 | Screening | 116 | 70 | 88 | 16 |
| EDLA 2.5% left | | | | | | |
| | | 96 h post-inj. | 133 | 78 | 62 | 24 |

^{a}Clinically notable abnormality is bolded.
^{b}This value is the lowest of five clinically notable DBP values recorded on the day of injection.
^{c}Occurred at 1 hour post-injection.

As shown in Table F2, the duration of sensory block for 1.25% 40K EDLA was notably longer in area C (1.53±0.82) compared to the other areas (0.16±0.10, 0.37±0.17, and 0.98±0.97 days in areas A, B and D respectively). With the 0.5% AB treatment, the duration was similar in areas B (0.38±0.35 days) and C (0.33±0.20 days). A shorter duration following 0.5% AB treatment was observed in area A (0.05±0.02 days) and area D (0.04±0.02 days). Thus, overall, duration of analgesia for AB was shorter than that seen for 40K EDLA in areas C and D, but approximately equal to that seen in areas A and B.

The mean duration of analgesia/anesthesia in area C was 1.80 days (across doses) for 40K EDLA and 0.62 days for AB. The 1.25% 40K EDLA concentration was selected for comparison of duration with AB. Duration of analgesia was longer for 1.25% 40K EDLA compared to AB (1.5 days versus 0.3 days). The 2.5% 40K EDLA group demonstrated a longer mean duration of sensory block compared to the lower concentrations (0.625%=1.7 days; 1.25%=1.5 days; 2.5%=2.2 days; FIG. F2).

The 1.25% concentration of 40K EDLA was selected from Part 1 for comparison with the equivalent concentration of 40K IDLA in Part 2. The time course of the analgesia is shown in FIG. F3. The results in area C for Part 2 showed that onset of analgesia was similar for 40K EDLA versus 40K IDLA, occurring within 6 hours in 100% of 40K EDLA blocks versus 67% in the 40K IDLA blocks. Onset of analgesia occurred within 30 minutes in 67% of the blocks for both 1.25% 40K EDLA and 1.25% 40K IDLA. Duration of block in area C was similar in both groups (0.84 days for 1.25% 40K EDLA and 1.09 days for 1.25% 40K IDLA). Table F2 summarizes the results.

The 1.25% 40K EDLA had a slightly faster onset in areas C and D (67% of blocks occurred in under 6 hours) in comparison to areas A and B (33% of the blocks occurred in under 6 hours). Across doses, onset of block occurred with 40K EDLA within 6 hours in 78% of the blocks in area C compared to within 30 minutes for 100% of blocks with 0.5% AB.

With 1.25% 40K EDLA, the duration of analgesia/anesthesia was notably longer in area C (1.5 days versus 0.16, 0.37 and 0.98 days in areas A, B and D respectively). Across doses the duration of analgesia/anesthesia was longer in the 40K EDLA sensory blocks versus that in the AB group. The mean duration of analgesia in the 40K EDLA blocks ranged from 1.53 days to 2.23 days with the longest duration noted at the highest 40K EDLA concentration (0.625%=1.65 days, 1.25%=1.53 days and 2.5%=2.23 days). The mean duration of analgesia in the AB blocks ranged from 0.33 days to 1.00 days.

### Onset and Duration of Temperature Perception Block

Temperature perception block was assessed as set forth in Example D, at baseline, and at post-injection hours 0.5, 1, 2, 3 and 6. The first self-evaluation was performed at 12 hours post-injection and thereafter approximately every 12 hours following the investigator's assessment at each daily return visit. Self-assessments continued once in the morning and again in the evening for 14 consecutive days post-injection, regardless of offset. Blockade of temperature perception was rated on a scale of 0-1, with 0=“Yes” (a change in temperature was perceived), and 1=“No” (no change in temperature was perceived). The data presented below are those from area C, the area that was shown to provide the longest duration of analgesia. The results are set forth in Table F3:

TABLE F3
Onset and Duration of Temperature Perception Block in Area C
Study Part 1

| Time to Onset | Treatment Pair | | Treatment Pair | | Treatment Pair | |
|---|---|---|---|---|---|---|
| of Temperature | 40K EDLA | AB | 40K EDLA | AB | 40K EDLA | AB |
| Perception | 0.625% | 0.5% | 1.25% | 0.5% | 2.5% | 0.5% |
| Block (No., %) | N = 3 | | N = 3 | | N = 3 | |
| ≦30 min | 0 | 3 (100%) | 0 | 3 (100%) | 2 (67%) | 3 (100%) |
| >30 min-1 hr | 0 | 0 | 0 | 0 | 0 | 0 |
| >1-2 hrs | 1 (33%) | 0 | 0 | 0 | 1 (33%) | 0 |
| >2-3 hrs | 0 | 0 | 1 (33%) | 0 | 0 | 0 |
| >3-6 hrs | 0 | 0 | 1 (33%) | 0 | 0 | 0 |
| >6 hrs | 2 (67%) | 0 | 1 (33%) | 0 | 0 | 0 |

Duration (days)

| | | | | | | |
|---|---|---|---|---|---|---|
| Mean | 1.11 | 0.95 | 3.10 | 0.37 | 0.03 | 0.61 |
| (SE) | (0.61) | (0.16) | (0.55) | (0.21) | (0.01) | (0.52) |
| Range | 0.02-2.15 | 0.67-1.22 | 2.25-4.13 | 0.02-0.73 | 0.01-0.06 | 0.01-1.66 |

Study Part 2

| Time to Onset | | |
|---|---|---|
| of Temperature | 40K EDLA | 40K IDLA |
| Perception | 1.25% | 1.25% |
| Block (No., %) | N = 3^{b} | N = 3 |
| ≦30 min | 1 (33%) | 1 (33%) |
| >30 min-1hr | 0 | 0 |
| >1-2 hrs | 0 | 1 (33%) |
| >2-3 hrs | 1 (33%) | 0 |
| >3-6 hrs | 1 (33%) | 0 |
| >6 hrs | 0 | 1 (33%) |
| Duration (days) | | |
| Mean | 2.85 | 0.49 |
| (SE) | (1.93) | (0.36) |
| Range | 0.01-6.55 | 0.11-1.21 |

The results for temperature perception block were similar to those for analgesia; 40K EDLA had a slightly earlier onset (100% within 6 hours for 40K EDLA versus 67% within 6 hours for 40K IDLA). As can be ascertained from the results set forth in Table F3, onset of temperature perception block in area C was earlier for AB across all doses (100% within 30 minutes) than for 40K EDLA across all doses (22% within 30 minutes). As shown in FIG. F4, onset of temperature perception block within 6 hours in area C was observed more reliably with progressively higher concentrations of 40K EDLA (33% for 0.625%, 67% for 1.25% and 100% for 2.5%). The 2.5% 40K EDLA group had 100% response rate within 2 hours.

Across doses, the duration of temperature perception block was longer in area C for 40K EDLA versus AB (1.41 days versus 0.64 days, respectively). Blockade of temperature perception in area C was longer for 1.25% 40K EDLA in comparison to the higher and lower concentrations (0.625% 40K EDLA=1.11 days; 1.25%=3.10 days; and 2.5%=0.03 days) (Table F3). The mean duration of temperature block across the 40K EDLA doses ranged from 0.03 to 3.10 days for the 40K EDLA group, while for AB the mean duration of sensory block ranged from 0.37 to 0.95 days. The short duration of temperature perception block in the 2.5% 40K EDLA group was due in part to the utilization of only the time of the initial temperature perception block for reporting the duration of the block. As shown in FIG. F5, blockade of temperature perception in area C was longer for 1.25% 40K EDLA versus 1.25% 40K IDLA (2.85 days for 40K EDLA versus 0.49 days for 40K IDLA).

Incidence of Analgesia/Anesthesia

In evaluating Incidence of analgesia/anesthesia, sensory block was rated as analgesia if the subject reported feeling two (2) or three (3) of the pinpricks as touch/pressure. Sensory block was rated as anesthesia if the subject reported no sensation in response to pinprick. The number (%) of subjects who experienced analgesia versus anesthesia at any time point was calculated. Percent of unsuccessful sensory blocks was defined as the percent of blocks in which neither anesthesia nor analgesia were demonstrated. The percent of unsuccessful blocks at any time-point was calculated. The results are shown in Table F4:

TABLE F4
Incidence of Analgesia^{a}/Anesthesia^{b} in Area C
Study Part 1

| | | | | | |
|---|---|---|---|---|---|
| 0.625% 40K | | 1.25% 40K | | 2.5% 40K | 0.5% |
| EDLA | 0.5% AB | EDLA | 0.5% AB | EDLA | AB |

| | | |
|---|---|---|
| N = 3 | N = 3 | N = 3 |

No. (%) with analgesia^{a}

| | | | | | |
|---|---|---|---|---|---|
| 3 | 3 | 3 | 3 | 3 | 3 |
| (100%) | (100%) | (100%) | (100%) | (100%) | (100%) |

No. (%) with anesthesia^{b}

| | | | | | |
|---|---|---|---|---|---|
| 2 | 1 | 1 | 1 | 2 | 2 |
| (67%) | (33%) | (33%) | (33%) | (67%) | (67%) |

Study Part 2

| | |
|---|---|
| 1.25% 40K EDLA | 1.25% 40K IDLA |
| N = 3 | N = 3 |

No. (%) with analgesia^{a}

| | |
|---|---|
| 3 | 3 |
| (100%) | (100%) |

No. (%) with anesthesia^{b}

| | |
|---|---|
| 2 | 1 |
| (67%) | (33%) |

^{a}Subjects felt 2 or 3 of the 3 pinpricks as touch/pressure
^{b}Subjects had not felt any of 3 pinpricks

The incidence of analgesia in area C was the same for 40K EDLA and AB as shown in Table F4 (analgesia=100% for both groups). The incidence of anesthesia was slightly higher for 40K EDLA (across all dosing groups, 56% incidence of anesthesia for 40K EDLA and 44% for AB). Among 40K EDLA dose groups, the incidence of block in area C was fairly consistent. Analgesia occurred in 100% of the blocks for all 40K EDLA doses. Anesthesia occurred slightly less frequently in the 1.25% group (33%) compared to subjects in the 0.625% and 2.5% groups (67%).

The 1.25% concentration demonstrated the same rate of analgesia (100%) in area C for both the 40K EDLA and 40K IDLA groups. The incidence of anesthesia was higher in the 1.25% 40K EDLA group (67%) versus the 1.25% 40K IDLA group (33%). None of the sensory blocks administered in this study were unsuccessful in area C.

In area A during Part 1 of the study, 67% of the blocks across doses of 40K EDLA were unsuccessful and 11% of the blocks for AB were unsuccessful. In area A for Part 2 of the study, 33% of the blocks were unsuccessful for 1.25% 40K EDLA and 67% of the blocks were unsuccessful for 1.25% 40K IDLA. In area B of the study during Part 1, 11% of the blocks that were unsuccessful for both 40K EDLA and AB. In area B during Part 2 of the study, there were no unsuccessful blocks in the 1.25% 40K EDLA group, whereas 67% of the blocks were unsuccessful in the 1.25% 40K IDLA group. Finally, in area D, there were no unsuccessful blocks observed during Part 1 of the study. During Part 2 of the study, no unsuccessful blocks were seen in area D for the 1.25% 40K EDLA group; 33% of the blocks were unsuccessful in area D for the 1.25% 40K IDLA group.

Degree of Numbness

Degree of numbness was assessed by asking subjects to rate the degree of numbness following touch to the sensory blocked areas on the back of the hand. Degree of numbness (defined as the distribution of numbness ratings at each time point) was based on an 11-point rating scale; 0 equals not numb at all and 10 equals totally numb. Degree of numbness was assessed at Baseline, and at post-injection hours 0.5, 1, 2, 3 and 6. The first self-evaluation was performed at 12 hours post-injection and thereafter approximately every 12 hours following the investigator's assessment at each daily return visit for 14 consecutive days post-injection, regardless of offset.

The peak numbness score for the 1.25% group was seen at Day 1 post-injection while the peak numbness score for AB was observed at 30 minutes post-injection. Across 40K EDLA doses, the mean peak numbness score in area C was 7.89 at 1 day post-injection, and was approximately equal to that seen with AB, peak numbness score of 9.33, which occurred earlier, at 30 minutes post-injection. The highest mean numbness scores in area C and the time of peak numbness for each dose group were as follows: 0.625% 40K EDLA=score of 7 at both 12 hours and 1 day post-injection; 1.25% 40K EDLA=score of 9 at Day 1 and 2.5% 40K EDLA=score of 7.67 at Day 1. The peak numbness score was seen later for the 40K EDLA groups.

As shown in FIG. F6, the peak numbness scores in area C for 1.25% 40K EDLA and 1.25% 40K IDLA were quite similar. The mean numbness score for 1.25% 40K EDLA was 8 and occurred at both 12 hours and 1 day post-injection, compared to a score of 8.33 for 40K IDLA, which occurred 6 hours post-injection. Thus, while the peak numbness scores were similar, the peak numbness score was achieved much sooner in the 40K IDLA group.

Mechanical Touch Detection Threshold

Mechanical Touch Detection Threshold was defined as the lowest force or number of a Von Frey Hair (VFH) that produced a sensation of touch or pressure. Mechanical Touch Detection Threshold was determined using 20 progressively rigid Von Frey Hairs (Somedic A/B, Stockholm, Sweden). Each of the four (4) designated areas on the top of the hand were stimulated three times with each VFH, starting with VFH No. 1.65 (least rigid) up to VFH No. 6.65 (most rigid). The lowest VFH number in which two (2) of the three (3) stimulations were detected (sensed as touch or pressure) was recorded. If VFH No. 6.65 had not produced any sensation of touch or pressure (two [2] out of three [3] stimulations), a value of seven (7) was assigned. Mechanical Touch Detection Threshold was assessed at baseline, and at post-injection hours 0.5, 1, 2, 3 and 6 by the principal investigator; every 24 hours thereafter until offset; and on Day 7 and Day 14 regardless of offset.

In Part 1 of the study, the 40K EDLA sensory blocks and AB sensory blocks had similar Mechanical Touch Detection Threshold scores. Across doses the peak Mechanical Touch Detection Threshold score in area C for the 40K EDLA groups was 5.10 occurring at Day 1 post-injection. The peak Mechanical Touch Detection Threshold score for AB was 5.04 occurring at 1 hour post-injection. Thus, the peak Mechanical Touch Detection Threshold score was seen in the AB group with an earlier onset than was seen in the 40K EDLA group. Similar Mechanical Touch Detection Threshold scores were obtained across the three 40K EDLA dose groups: 0.625% 40K EDLA=4.78 at Day 1; 1.25% 40K EDLA=5.54 at Day 1; and 2.5% 40K EDLA=4.98 at Day 1.

The Mechanical Touch Detection Threshold for 40K EDLA and 40K IDLA were as shown in FIG. F7. The peak-Mechanical Touch Detection Threshold score for 1.25% 40K EDLA was 4.85 and occurred on Day 1, compared to a score of 4.36 for 1.25% 40K IDLA, which occurred 6 hours post-injection. Thus, a similar peak was seen in the 40K EDLA and 40K IDLA groups, with a longer latency onset to peak score observed in the 40K EDLA group. Similar Mechanical Touch Detection Threshold scores were observed in the 40K EDLA and AB groups. Across doses, the peak Mechanical Touch Detection Threshold score for 40K EDLA groups in area C was 5.54 while the peak Mechanical Touch Detection Threshold score for AB groups was 5.04. Similar Mechanical Touch Detection Threshold scores were obtained for the three 40K EDLA dose groups (0.625%=4.78, 1.25%=5.54 and 2.5%=4.98).

Pharmacokinetic Results

Plasma bupivacaine and dexamethasone concentrations over time were determined for 40K EDLA- and 40K IDLA-treated subjects in Part 2. Pharmacokinetic parameters (Cₘₐₓ, Tₘₐₓ, and AUC) were calculated from plasma concentrations of 40K EDLA and 40K IDLA.

Subjects had blood drawn pre-dose (baseline), at three (3) and six (6) hours post-injection, and approximately every 24 hours until the offset of the block was determined. Dexamethasone and bupivacaine concentrations were determined using liquid chromatography. The calibration ranged from 0.05 to 300 ng/mL for dexamethasone and 5.00 to 300 ng/mL for bupivacaine, where the limit of quantitation was 0.05 ng/mL for dexamethasone and 5.00 ng/mL for bupivacaine.

Data on plasma bupivacaine concentrations are summarized by treatment group in FIG. F8. Mean plasma bupivacaine concentration versus time curves for 40K EDLA and 40K IDLA were markedly different from one another. The 1.25% 40K EDLA group had an early mean peak of 92.67 ng/ml at 3 hours post-injection. This peak level (which was seen earlier than in the 40K IDLA group) was maintained at approximately this level at 6 hours and Day 2 post-injection, but with a drop to 45.73 ng/ml at Day 1 post-injection. Plasma bupivacaine levels in the 40K EDLA group were still elevated at Day 4 post-injection at 52.85 ng/ml. In the one subject sample collected at Days 5 and 6, the plasma bupivacaine levels had returned to near-baseline levels of 28.2 ng/ml (day 5) and 19 ng/ml (day 6).

Following injection with 1.25% 40K IDLA, plasma levels of bupivacaine had a mean peak value of 106.03 ng/ml at 24 hours (FIG. F8). Elevated levels of plasma bupivacaine were still observed at 2 days post-injection (60.23 ng/ml). At Days 3 and 4 post-injection; however, the bupivacaine levels had decreased to 14.8 ng/ml and 6.28 ng/ml, respectively.

Plasma dexamethasone concentrations were undetectable at most of the timepoints that were measured following injection with 1.25% 40K EDLA or 40K IDLA. The mean plasma dexamethasone concentrations in the 40K EDLA group that were detectable were observed at 3 hours (0.1 ng/ml), 6 hours (0.12 ng/ml) and 2 days (0.02 ng/ml) post-injection. Plasma bupivacaine pharmacokinetic parameters are summarized in Table F5 below:

TABLE F5
Bupivacaine Pharmacokinetic Parameters (Mean (+/−SE)

Study Part 2

| | 40K EDLA | 40K IDLA |
|---|---|---|
| | 1.25% | 1.25% |
| | N = 3 | N = 3 |
| PK Parameter | | |
| Cₘₐₓ (ng/ml) | 153.03 (57.07) | 106.03 (38.18) |
| Tₘₐₓ (hr) | 27 (22.52) | 24 (0) |
| AUC (ng*hr/ml) | 6842.67 (3919.99) | 3333.67 (1196.93) |

As can be seen in Table F5, the time of occurrence of peak bupivacaine concentrations (Tₘₐₓ) was similar between the 40K EDLA and 40K IDLA groups (27 hours post-injection for the 40K EDLA group and 24 hours post-injection for the 40K IDLA group). However, the maximum concentration of drug (Cₘₐₓ) was higher in the 40K EDLA group (153.03 ng/ml for 40K EDLA group and 106.03 in the 40K IDLA group). This higher Cₘₐₓ in the 40K EDLA group and longer time at which the elevated concentrations of bupivacaine were maintained resulted in an increased total bupivacaine AUC in the 40K EDLA group in comparison to the 40K IDLA group. The mean total AUC for the 40K EDLA group was 6842.7 ng*hr/ml while the mean total AUC for the 40K IDLA group was 3333.7 ng*hr/ml.

No plasma dexamethasone pharmacokinetic parameters were reported for either the 40K EDLA or 40K IDLA groups in Part 2 of the study. The very low mean plasma dexamethasone concentrations that were detectable in the 40K EDLA group were observed following the injection at 3 hours (0.1 ng/ml), 6 hours (0.12 ng/ml) and 2 days (0.02 ng/ml).

Nerve Conduction Testing

To assess potential nerve damage, change from baseline (pre-injection) in amplitude and velocity of nerve conduction was assessed. Nerve conduction studies were conducted to determine the time course of neurophysiological effects of the drug, specifically, the amplitude, latency and distance of the neurophysiological sensory response over time, and the time to return of normal sensation. Nerve conduction studies were conducted on the right and/or left superficial radial nerves on the hand assigned to be injected with study medication. Skin temperature on the hands was measured using a standard practice/method. A minimum skin temperature of 32° C. was maintained throughout the conduct of nerve testing, and thermal packs was used to warm the hands if the temperature fell below the minimum. Two recording ring electrodes coated with conducting gel were placed over the base of the thumb and the stimulating electrode was placed over the wrist, approximately 2 cm proximal to the wrist. A ground electrode, coupled with electrode paste, was taped to the skin between the stimulating and recording electrodes. Using graded intensity stimuli, single electrical pulses, lasting no more than 1/1,000^{th} of a second (1 ms), were gradually increased in current until a maximal sensory response was obtained. The intensity was then increased slightly to ensure supramaximal stimulation, in accordance with standard practice.

Some variation in the course of the superficial radial nerve and its branches was anticipated, in keeping with the anatomic variability of this nerve. Accordingly, disc recording electrodes in the first web space were affixed to the skin with tape. The response amplitude in the web space was to be five (5) microvolts or greater. If the response amplitude suggested that alternate sites were superior, the investigator varied the placement of the recording electrodes over the distal branches of the nerve. Once a reliable assessment montage was determined for a given subject, the identical montage was used throughout the course of the study to measure the latency and amplitude of the radial response. Sensory response amplitude, latency and distance [between stimulating and recording electrodes], as well as skin temperature, were recorded. In addition, placement of the stimulus and recording electrodes was described.

Nerve conduction testing was performed at baseline, 1, 6, and 24 hours post-injection, and thereafter, on Days 7 and 14; and at the 6-week follow up. If the results of the nerve conduction test were abnormal at the 6-week evaluation (outside of +/−20% of normal range), the tests were repeated at the 3- and 6-month follow-up visits. If the results were normal at the 6-week evaluation, no further nerve conduction tests were required. Changes in amplitude and/or velocity of nerve conduction were summarized by treatment. Post-injection vital signs were compared with baseline assessments using a paired t-test. Laboratory values recorded pre- and post-injection were analyzed using shift tables.

Table F6 provides data obtained with respect to changes in amplitude of nerve conduction:

TABLE F6
Radial Nerve Conduction - Amplitude (uV)
Study Part 1

| | Treatment Pair | | Treatment Pair | | Treatment Pair | |
|---|---|---|---|---|---|---|
| | 40K EDLA | AB | 40K EDLA | AB | 40K EDLA | AB |
| | 0.625% | 0.5% | 1.25% | 0.5% | 2.5% | 0.5% |
| | N = 3 | | N = 3 | | N = 3 | |
| Baseline Mean | 33 | 28.67 | 36.5 | 31 | 35.17 | 31 |
| Mean Change Hour 1 | −15.33* | −18.67 | −14.17 | −18.83* | −12.13 | −20.33 |
| Mean Change Hour 6 | −16.37* | −20.47* | −18.17* | −19.67* | −21.17 | −16** |
| Mean Change Hour 24 | −18* | −10.67 | −25.83* | −7.67 | −24.5 | −15** |
| Mean Change Day 7 | −4.83 | −1 | −9.83 | −3.33 | −12.5 | +0.17 |
| Mean Change Day 14 | −1.83 | +3 | −1.67 | +3 | −5.33 | +1 |

Study Part 2

| | Treatment Group | Treatment Group |
|---|---|---|
| | 1.25% 40K EDLA | 1.25% 40K IDLA |
| | N = 3 | N = 3 |
| Baseline Mean | 40 | 37.83 |
| Mean Change Hour 1 | −18.67 | −14.17 |
| Mean Change Hour 6 | −21.5* | −19.5** |
| Mean Change Hour 24 | −33.3^{a} | −21** |
| Mean Change Day 7 | −3.67 | −5.83 |
| Mean Change Day 14 | −7.5 | −2.33 |

*Significant at ≦0.05 level
**Significant at ≦0.01 level
^{a}Only two subjects were evaluated at Hour 24; the mean change compared to baseline mean is for the same two subjects.

Aqueous bupivacaine (0.5%), 40K EDLA, and 40K IDLA all resulted in diminished amplitude of the conducted impulse beginning at hour 1. In general, the effect on nerve conduction amplitude was greater for higher concentrations of 40K EDLA and was greater for 1.25% 40K EDLA than for 1.25% 40K IDLA. The effect of 0.5% AB was greater than any concentration of 40K EDLA at Hour 1, a difference that had reversed by hour 24.

Table F7 provides data obtained with respect to changes in velocity of nerve conduction:

TABLE F7
Radial Nerve Conduction - Velocity (cm/ms)
Study Part 1

| | Treatment Pair | | Treatment Pair | | Treatment Pair | |
|---|---|---|---|---|---|---|
| | 40K EDLA | AB | 40K EDLA | AB | 40K EDLA | AB |
| | 0.625% | 0.5% | 1.25% | 0.5% | 2.5% | 0.5% |
| | N = 3 | | N = 3 | | N = 3 | |
| Baseline Mean | 6.13 | 6.25 | 6.16 | 5.77 | 5.88 | 6.13 |
| Mean Change Hour 1 | +0.03 | −0.42 | +0.25 | +0.42 | +0.39 | +0.33 |
| Mean Change Hour 6 | +0.28 | −0.69 | +0.56 | +0.06 | +0.39 | +0.21 |
| Mean Change Hour 24 | +0.28 | −0.12 | −0.15 | +0.02 | +0.67 | −0.73 |
| Mean Change Day 7 | −0.42 | +0.02 | +0.14 | +0.23 | +0.02 | +0.03 |
| Mean Change Day 14 | +0.12 | 0 | −0.12 | +0.48* | 0 | 0 |

Study Part 2

| | Treatment Group | Treatment Group |
|---|---|---|
| | 1.25% 40K EDLA | 1.25% 40K IDLA |
| | N = 3 | N = 3 |
| Baseline Mean | 6.73 | 6.87 |
| Mean Change Hour 1 | −0.04 | −0.12 |
| Mean Change Hour 6 | −0.17 | −0.31 |
| Mean Change Hour 24 | 0^{a} | −0.07 |
| Mean Change Day 7 | −0.73 | −0.81 |
| Mean Change Day 14 | −0.46 | −0.45* |

*Significant at ≦0.05 level
^{a}Only two subjects with recorded value at Hour 24; the mean change is based on comparison for these two subjects only.

The effects on nerve conduction velocity were small for all agents and concentrations. This effect was slightly greater in the 40K EDLA than the 40K IDLA group, and the effect was correlated with increasing concentrations of 40K EDLA. For all treatment groups, the change in conduction and amplitude had resolved or nearly resolved at the Day 7 evaluation.

Conclusions

In general, 40K EDLA had a longer onset and duration of action than 0.5% AB for both analgesia and temperature perception block. The mean duration of analgesia/anesthesia in area C was 1.80 days (across doses) for 40K EDLA and 0.62 days for aqueous bupivacaine (across doses). Assessment area C provided the most consistent onset and longest duration among the 40K EDLA groups. Thus, the efficacy results reported were focused on the results obtained from area C. Dexamethasone was generally more effective in prolonging the action of 40K EDLA in measures of efficacy (i.e., 2.85 days of temperature perception block for 1.25% 40K EDLA group versus 0.49 days for 1.25% 40K IDLA group). The 40K EDLA group had a higher total systemic exposure to bupivacaine than did the 40K IDLA group (a mean total AUC of 6842 ng*hr/ml for 40K EDLA and 3333.7 ng*hr/ml for 40K IDLA). Thus, 40K EDLA in the 1.25% and 2.5% formulations appears to be a safe and effective method of producing local analgesia of extended duration.

Effect of Dexamethasone on Sensory Nerve Blocks

The results of Part 2 were designed to illustrate the potential effect that very low doses of dexamethasone can have on extending the duration and effectiveness of 40K EDLA. In Part 2 of the study, 1.25% 40K EDLA produced a more rapid onset of analgesia/anesthesia (100% of the subjects within 6 hours) and had a similar duration of action (mean=0.84 days) compared to 1.25% 40K IDLA (onset=67% within 6 hours; mean duration=1.09 days).

Results of the time to onset of temperature perception block (somesthetic test) were similar to those for analgesia; 40K EDLA had a slightly earlier onset (100% within 6 hours for 40K EDLA versus 67% within 6 hours for 40K IDLA). The duration of temperature perception block was much longer for 40K EDLA in comparison to 40K IDLA (2.85 days for 40K EDLA versus 0.49 days for 40K IDLA).

40K EDLA produced a similar degree of numbness score (peak numbness score=8) compared to 40K IDLA (peak numbness score=8.33).

The same rate of analgesia (100%) was noted in both 40K EDLA and 40K IDLA. The incidence of anesthesia was slightly higher in the 40K EDLA group (67%) versus the 40K IDLA group (33%).

In a similar fashion the Mechanical Touch Detection Threshold for 40K EDLA and 40K IDLA produced similar responses. The peak Mechanical Touch Detection Threshold score for 40K EDLA was 4.85 and occurred on Day 1 post-injection, while the peak Mechanical Touch Detection Threshold score for 40K IDLA was 4.36 which occurred 6 hours post-injection. Thus, while the peak Mechanical Touch Detection Threshold scores were similar in the 40K EDLA and 40K IDLA groups, longer time needed to reach the peak score in the 40K EDLA group.

Summary of Safety

40K EDLA, 40K IDLA, and AB were all associated with a time-limited decrease in the amplitude of radial nerve conduction. Small changes were observed in the velocity of radial nerve conduction and were not judged to be of clinical significance, including the two statistically significant changes that appeared at the Day 14 evaluation in radial nerves exposed to 0.5% AB (in the 1.25% 40K EDLA/0.5% AB group) and 1.25% 40K IDLA.

### Example G

### The Sensory Blockade Characteristics of an Extended Duration Local Anesthetic (EDLA) and an Intermediate Duration Local Anesthetic (IDLA) When Administered to the Superficial Peroneal Nerve

An open-label, comparative, 2-part, dose-response study evaluated ascending dose levels of 120K EDLA and 40K EDLA to identify the effective dose. At the effective dose, the optimal formulation would provide a 3- to 5-day duration of sensory block. The role of dexamethasone in extending the duration of bupivacaine activity was also evaluated. The five treatments were 120K EDLA and 40K EDLA, prepared in accordance with Example 2, and 120K IDLA and 40K IDLA (prepared in accordance with Example 1), and Aqeuous Bupivacine (AB). The test drugs and concentrations, all of which were administered in 3-milliliter (mL) injections, are shown in Table G1 below.

TABLE G1

| Drug and Concentration* | Dose Form | Unit Strength (each mL) | | Total Dose (3 mL) |
|---|---|---|---|---|
| 120K EDLA 0.625% | Suspension | Bupivacaine | 4.5 mg/mL | 13.5 mg |
| | | Dexamethasone | 2.5 μg/mL | 7.5 μg |
| 120K EDLA 1.25% | Suspension | Bupivacaine | 9.0 mg/mL | 27.0 mg |
| | | Dexamethasone | 5.0 μg/mL | 14.5 μg |
| 120K EDLA 2.5% | Suspension | Bupivacaine | 18.0 mg/mL | 54.0 mg |
| | | Dexamethasone | 10.0 μg/mL | 30.0 μg |
| 40K EDLA 0.312% | Suspension | Bupivacaine | 2.3 mg/mL | 7.0 mg |
| | | Dexamethasone | 1.2 μg/mL | 3.7 μg |
| 40K EDLA 0.625% | Suspension | Bupivacaine | 4.5 mg/mL | 13.5 mg |
| | | Dexamethasone | 2.5 μg/mL | 7.5 μg |
| 40K EDLA 1.25% | Suspension | Bupivacaine | 9.0 mg/mL | 27.0 mg |
| | | Dexamethasone | 5.0 μg/mL | 14.5 μg |
| 40K EDLA 2.5% | Suspension | Dexamethasone | 18.0 mg/mL | 54.0 mg |
| | | Bupivacaine | 10.0 μg/mL | 30.0 μg |
| 120K IDLA 1.25% | Suspension | Bupivacaine | 9.0 mg/mL | 27.0 mg |
| 40K IDLA 1.25% | Suspension | Bupivacaine | 9.0 mg/mL | 27.0 mg |
| | | | | 15.0 μg |
| AB 0.5% | Solution | Bupivacaine | 5 mg/mL | 15.0 mg |

The 40K IDLA formulation was included to assess the role of dexamethasone in extending the duration of bupivacaine local activity. Procedures for testing 120K EDLA versus (vs) 120K IDLA and 40K EDLA vs 40K IDLA were slightly different. Unilateral injection of 120K EDLA or 120K IDLA permitted assessment of plasma concentrations of bupivacaine and dexamethasone. Plasma concentrations were not assessed in subjects receiving bilateral injections of 40K EDLA and 40K IDLA. Treatments administered are shown in Table G2 below.

TABLE G2
Test and Reference Treatments for the Comparison
Study Part 1^{a}
Bilateral Injections

| | |
|---|---|
| Study Drug and Dose | Reference |

| | | |
|---|---|---|
| 120K EDLA | 40K EDLA | AB |
| (3 mL) | (3 mL) | (3 mL) |
| — | 0.312% | 0.5% |
| 0.625% | 0.625% | 0.5% |
| 1.25% | 1.25% | 0.5% |
| 2.5% | 2.5% | 0.5% |

Study Part 2
Unilateral Injections

| | |
|---|---|
| 120K EDLA | 120K IDLA |
| (3 mL) | (3 mL) |
| 1.25% | 1.25% |

Bilateral Injections^{b}

| | |
|---|---|
| 40K EDLA | 40K IDLA |
| (3 mL) | (3 mL) |
| 1.25% | 1.25% |

^{a}Subjects received 120K EDLA or 40K EDLA in one foot plus AB in the other
^{b}The 40K EDLA and 40K IDLA groups comprised 6 subjects who received EDLA in one foot and IDLA in the other.

For each superficial peroneal nerve block administered, the intermediate branch of the superficial peroneal nerve was made prominent by the maximum plantar flexion and slight adduction of the foot and its superficial course was marked where it is most easily identified, just medial and slightly distal to the lateral (fibular) malleolus. The needle was redirected towards the medial malleolus and advanced 2-4 centimeter (cm) as an additional medication was injected subcutaneously to anesthetize the medial branch of the superficial peroneal nerve.

In Part 1, subjects received 120K EDLA (at 0.625%, 1.25% or 2.5%) as a superficial peroneal nerve block to one foot and AB 0.5% as a superficial peroneal nerve block to the opposite foot. For evaluating the 40K EDLA formulation, subjects received 40K EDLA (0.312%, 0.625%, 1.25% or 2.5%) as a superficial peroneal nerve block to one foot and AB 0.5% as a superficial peroneal nerve block to the opposite foot.

The 1.25% concentration was selected for comparison of 40K EDLA to 120K EDLA and to 40K IDLA in Part 2. Subjects received unilateral injections of 120K EDLA or IDLA at 1.25% in the left foot. Additional subjects received bilateral injections of 1.25% 40K EDLA in one foot and 1.25% 40K IDLA in the other foot.

Efficacy measurements were onset and duration of analgesia (with or without anesthesia), incidence of analgesia (with or without anesthesia), onset and duration of temperature perception block, degree of numbness, number (%) of unsuccessful sensory blocks, and pharmacokinetics/pharmacodynamic meausures. Safety variables included pain upon injection.

Onset and Duration of Analgesia (with or without Anesthesia)

Sensory blockade was assessed by lightly tapping the skin on the dorsum of the foot, using the dull end of a dental needle (or similar type needle). The area above the 3rd and 4th metatarsals was designated as the primary test area. Additional area(s) were identified as demonstrating sensory block; these areas were designated secondary test areas. The primary and secondary areas demonstrating sensory block were marked with a surgical pen to designate the pinprick test areas. Pinprick tests were then conducted consistently within these sites.

The primary and secondary areas were tested using pinprick three times and the subject was asked how many pinpricks were felt. The density of the sensory block was rated on a 0-2 scale, with 0=anesthesia, 1=analgesia, and 2=no block. Ratings were scored as 0=subject felt 0 (out of 3) pinpricks; 1=subject felt 2 or 3 (out of 3) pinpricks as touch or pressure or subject felt 2 (out of 3) pinpricks, 1 as touch or pressure, and 1 as sharp; and, 2=subject felt 2 or 3 (out of 3) pinpricks as sharp.

Onset of analgesia (with or without anesthesia) was defined as the first time at which pinprick testing on the top of the foot demonstrated analgesia (touch/pressure) or anesthesia (no pinpricks felt). Pinprick testing for onset of sensory block was performed at pre-dose (baseline) and approximately 30 minutes, 1, 2, 3 and 6 hours post-injection.

Across all doses (Parts 1 and 2), onset of block occurred within ≦3 hours in 67% of blocks for 40K EDLA, compared to 100% of blocks for AB, and 4% of blocks for 120K EDLA. For the 1.25% concentration, onset of block occurred within ≦3 hours in 100% of 40K EDLA blocks vs 11% of 120K EDLA blocks (FIGS. G1 and G2). Onset of block was faster for higher vs lower concentrations of 40K EDLA, with onset occurring in ≦3 hours in 67% of blocks for 0.625% and 2.5% concentrations, and in 100% of blocks for the 1.25% concentration. These results are summarized in Table G3.

Onset of block was faster for 1.25% 40K EDLA compared to 1.25% 40K IDLA, occurring in ≦1 hour in 33% of 40K EDLA blocks vs 0% of the 40K IDLA blocks. As shown in FIG. G3 and Table G3, onset was ≦3 hours in 83% of 40K EDLA blocks vs 33% of 40K IDLA blocks. No response was observed for 1.25% 120K EDLA and 1.25% 120K IDLA in Part 2 of the study. There were no obvious reasons for this finding. One subject (Subject 21) received a suboptimal injection (drug not injected 1 hour after suspension was prepared). Plasma bupivacaine concentrations estimated for these subjects were below the limits of detection.

TABLE G3
Onset of Analgesia (with or without anesthesia)^{a}
Study Part 1

| | Treatment Pair | | Treatment Pair | | Treatment Pair | |
|---|---|---|---|---|---|---|
| | 120K EDLA | AB | 120K EDLA | AB | 120K EDLA | AB |
| Time to Onset of Analgesia | 0.625% | 0.5% | 1.25% | 0.5% | 2.5% | 0.5% |

| | | | |
|---|---|---|---|
| (+/−anesthesia) | N = 6 | N = 9 | N = 6 |

Number (%) Subjects

| | | | | | | |
|---|---|---|---|---|---|---|
| ≦30 min | 0 | 4 (67%) | 1 (11%) | 8 (89%) | 0 | 6 (100%) |
| >30 min ≦ 1 h | 0 | 2 (33%) | 0 | 0 | 0 | 0 |
| >1 ≦ 2 h | 0 | 0 | 0 | 0 | 0 | 0 |
| >2 ≦ 3 h | 0 | 0 | 0 | 0 | 0 | 0 |
| >3 ≦ 6 h | 0 | 0 | 1 (11%) | 0 | 1 (17%) | 0 |
| >6 h | 3 (50%) | 0 | 4 (44%) | 1 (11%) | 5 (83%) | 0 |

Study Part 1

| | Treatment Pair | | Treatment Pair | | Treatment Pair | | Treatment Pair | |
|---|---|---|---|---|---|---|---|---|
| | 40K EDLA | AB | 40K EDLA | AB | 40K EDLA | AB | 40K EDLA | AB |
| Time to Onset of Analgesia | 0.312% | 0.5% | 0.625% | 0.5% | 1.25% | 0.5% | 2.5% | 0.5% |

| | | | | |
|---|---|---|---|---|
| (+/−anesthesia) | N = 3 | N = 6 | N = 3 | N = 3 |

Number (%) Subjects

| | | | | | | | | |
|---|---|---|---|---|---|---|---|---|
| ≦30 min | 0 | 1 (33%) | 1 (17%) | 3 (50%) | 1 (33%) | 3 (100%) | 0 | 1 (33%) |
| >30 min ≦ 1 h | 0 | 2 (67%) | 0 | 3 (50%) | 1 (33%) | 0 | 0 | 2 (67%) |
| >1 ≦ 2 h | 0 | 0 | 1 (17%) | 0 | 0 | 0 | 1 (33%) | 0 |
| >2 ≦ 3 h | 0 | 0 | 2 (33%) | 0 | 1 (33%) | 0 | 1 (33%) | 0 |
| >3 ≦ 6 h | 1 (33%) | 0 | 1 (17%) | 0 | 0 | 0 | 1 (33%) | 0 |
| >6 h | 2 (67%) | 0 | 1 (17%) | 0 | 0 | 0 | 0 | 0 |

Study Part 2

Treatment Pair

| | | | | |
|---|---|---|---|---|
| | 120K EDLA | 120K IDLA | 40K EDLA | 40K IDLA |
| Time to Onset of Analgesia | 1.25% | 1.25% | 1.25% | 1.25% |
| (+/−anesthesia) | N = 3 | N = 3 | N = 6^{b} | N = 6 |

Number (%) Subjects

| | | | | |
|---|---|---|---|---|
| ≦30 min | 0 | 0 | 0 | 0 |
| >30 min ≦ 1 h | 0 | 0 | 2 (33%) | 0 |
| >1 ≦ 2 h | 0 | 0 | 1 (17%) | 1 (17%) |
| >2 ≦ 3 h | 0 | 0 | 2 (33%) | 1 (17%) |
| >3 ≦ 6 h | 0 | 0 | 1 (17%) | 2 (33%) |
| >6 h | 0 | 0 | 0 | 0 |

Note:
Columns resulting in fewer than 100% of subjects represented were due to unsuccessful sensory blocks (subjects had not experienced analgesia (with or without anesthesia)).
^{a}Analgesia = subjects who felt 2/3 or 3/3 pinpricks as touch/pressure. Anesthesia = subjects who felt 0/3 pinpricks.
^{b}The 40K EDLA and 40K IDLA groups comprised 6 subjects who received EDLA in one foot and IDLA in the other.

Duration of analgesia was defined as the time between onset of analgesia (with or without anesthesia) and time when there was a return of sensation of sharpness to pinpricks. Subjects returned to the study site approximately every 24 hours post-injection for pinprick testing by the evaluator until the offset of sensory blockade was determined. Assessments were performed once every 12 hours until the offset of block, and once every 24 hours thereafter for a total of 14 days, regardless of offset. Across doses, duration of block (analgesia with or without anesthesia) was longer for 40K EDLA compared to AB (40K EDLA=2.3 days vs AB=0.5 days), and was shorter than that observed for 120K EDLA (2.8 days). Duration of analgesia was longer for 1.25% 40K EDLA than for 1.25% 120K EDLA (3.1 days vs 1.7days), and both formulations had longer duration than the reference drug (from 0.2 to 0.6 days). Table G4 summarizes these results.

A longer mean duration of sensory block was observed for the 1.25% and 2.5% concentrations of 40K EDLA (3 mL) compared to the lower concentrations (0.312%=1.2 days; 0.625%=2.3 days; 1.25%=3.1 days; 2.5%=2.5 days; see FIGS. G1 and G2). The 2.5% concentration of 120K EDLA resulted in the longest mean duration of block (4.2 days). Duration of sensory block for individual subjects receiving 120K EDLA was prolonged. Six (6) subjects who received 1.25% and 2.5%120K EDLA had not experienced return of normal sensation by study Day 14.

Duration of analgesia was longer for 1.25% 40K EDLA (3 mL) compared to 1.25% 40K IDLA (2.1 days vs 0.6 days, respectively). As shown in FIG. G3, the mean pinprick scores showed 40K EDLA set up faster than 40K IDLA (2 hours vs 3 hours) and lasted considerably longer (day 3 vs hour 12). Duration of block for 40K IDLA and AB (across doses for 40K/AB treatment pairs) was similar (range, 0.1-0.8 days vs 0.2-0.8 days, respectively).

TABLE G4
Duration of Analgesia (with or without anesthesia)^{a} (Mean and Range (+/−SE))
Study Part 1

| | Treatment Pair | | Treatment Pair | | Treatment Pair | |
|---|---|---|---|---|---|---|
| | 120K EDLA | AB | 120K EDLA | AB | 120K EDLA | AB |
| | 0.625% | 0.5% | 1.25% | 0.5% | 2.5% | 0.5% |
| | N = 6 | | N = 9 | | N = 6 | |
| Duration (days) | | | | | | |
| Mean | 2.1 | 0.4 | 1.7 | 0.6 | 4.2 | 0.5 |
| (SE) | (1.1) | (0.1) | (0.5) | (0.1) | (2.3) | (0.1) |
| Range | 0.7, 4.2 | 0.4, 0.7 | 0.1, 3.2 | 0.2, 1.3 | 0, 13.9 | 0.2, 0.8 |

Study Part 1

| | Treatment Pair | | Treatment Pair | | Treatment Pair | | Treatment Pair | |
|---|---|---|---|---|---|---|---|---|
| | 40K EDLA | AB | 40K EDLA | AB | 40K EDLA | AB | 40K EDLA | AB |
| | 0.312% | 0.5% | 0.625% | 0.5% | 1.25% | 0.5% | 2.5% | 0.5% |
| | N = 3 | | N = 6 | | N = 3 | | N = 3 | |
| Duration (days) | | | | | | | | |
| Mean | 1.2 | 0.6 | 2.3 | 0.3 | 3.1 | 0.2 | 2.5 | 0.8 |
| (SE) | (0.5) | (0.1) | (0.2) | (0.03) | (0.6) | (0.1) | (1.7) | (0.03) |
| Range | 0.3, 1.9 | 0.4, 0.8 | 1.8, 2.7 | 0.2, 0.4 | 2.3, 4.3 | 0.2, 0.4 | 0.1, 5.8 | 0.7, 0.8 |

Study Part 2

Treatment Pair^{b}

| | 120K EDLA | 120K IDLA | | 40K IDLA |
|---|---|---|---|---|
| | 1.25% | 1.25% | 40K EDLA | 1.25% |
| | N = 3 | N = 3 | 1.25% | N = 6 |
| Duration (days) | | | | |
| Mean | 0 | 0 | 2.1 | 0.6 |
| (SE) | (0) | (0) | (0.5) | (0.1) |
| Range | 0 | 0 | 0.02, 3.3 | 0.3, 0.8 |

Note:
Columns resulting in fewer than 100% of subjects represented were due to unsuccessful sensory blocks (subjects had not experienced analgesia (with or without anesthesia)).
^{a}Analgesia = subjects who felt 2/3 or 3/3 pinpricks as touch/pressure. Anesthesia = subjects who felt 0/3 pinpricks.
^{b}The 40K EDLA and 40K IDLA groups comprised 6 subjects who received EDLA in one foot and IDLA in the other.

Incidence of Analgesia with or without Anesthesia

In evaluating incidence of analgesia (with or without anesthesia), sensory block was rated as analgesia if the subject reported feeling 2 or 3 of the pinpricks as touch/pressure. Sensory block was rated as anesthesia if the subject reported no sensation in response to pinprick. The number (%) of subjects who experienced analgesia at any time point was calculated.

Across 40K EDLA concentrations, the incidences of analgesia and anesthesia were similar for EDLA and AB (analgesia=100%, and anesthesia=93% for both 40 EDLA and AB). The incidence of analgesia and anesthesia was more reliable for 40K EDLA. Analgesia with or without anesthesia occurred in 100% vs 67% of subjects for 40K vs 120K EDLA, respectively. Anesthesia occurred in 100% vs 22% of subjects for 40K vs 120K EDLA, respectively. These results are shown in Table G5.

TABLE G5
Incidence of Analgesia (with or without anesthesia)^{a}
Study Part 1 - 120K EDLA

| Treatment Pair | | Treatment Pair | | Treatment Pair | |
|---|---|---|---|---|---|
| EDLA | AB | EDLA | AB | EDLA | AB |
| 0.625% | 0.5% | 1.25% | 0.5% | 2.5% | 0.5% |

| | | |
|---|---|---|
| N = 6 | N = 9 | N = 6 |

Number (%) Subjects

| | | | | | | |
|---|---|---|---|---|---|---|
| Analgesia | 3 (50%) | 6 (100%) | 6 (67%) | 9 (100%) | 6 (100%) | 6 (100%) |
| (+/−anesthesia)^{a} | | | | | | |
| Anesthesia^{b} | 1 (17%) | 5 (83%) | 2 (22%) | 8 (89%) | 5 (83%) | 6 (100%) |

Study Part 1 - 40K EDLA

| Treatment Pair | | Treatment Pair | | Treatment Pair | | Treatment Pair | |
|---|---|---|---|---|---|---|---|
| EDLA | AB | EDLA | AB | EDLA | AB | EDLA | AB |
| 0.312% | 0.5% | 0.625% | 0.5% | 1.25% | 0.5% | 2.5% | 0.5% |

| | | | |
|---|---|---|---|
| N = 3 | N = 6 | N = 3 | N = 3 |

Number (%) Subjects

| | | | | | | | | |
|---|---|---|---|---|---|---|---|---|
| Analgesia | 3 (100%) | 3 (100%) | 6 (100%) | 6 (100%) | 3 (100%) | 3 (100%) | 3 (100%) | 3 (100%) |
| (+/−anesthesia)^{a} | | | | | | | | |
| Anesthesia^{b} | 2 (67%) | 3 (100%) | 6 (100%) | 5 (83%) | 3 (100%) | 3 (100%) | 3 (100%) | 3 (100%) |

Study Part 2 - EDLA/IDLA

Treatment Pair^{c}

| | | | |
|---|---|---|---|
| 120K EDLA | 120K IDLA | 40K EDLA | 40K IDLA |
| 1.25% | 1.25% | 1.25% | 1.25% |
| N = 3 | N = 3 | N = 6 | N = 6 |

Number (%) Subjects

| | | | | |
|---|---|---|---|---|
| Analgesia | 0 | 0 | 6 (100%) | 4 (67%) |
| (+/−anesthesia)^{a} | | | | |
| Anaesthesia^{b} | 0 | 0 | 6 (100%) | 4 (67%) |

^{a}Subjects felt 2 or 3 of the 3 pinpricks as touch/pressure, or 1 as touch/pressure and 1 as sharp.
^{b}Subjects had not felt any of 3 pinpricks
^{c}The 40K EDLA and 40K IDLA groups comprised the same 6 subjects who received EDLA in one foot and IDLA in the other.

The incidence of analgesia with or without anesthesia was reliable across 40K EDLA dose groups, occurring in 100% of all subjects and all dose groups. Anesthesia occurred in 67% of blocks for the 0.312% concentration, vs 100% for all other 40K EDLA concentrations) (see Table G5). The 120K formulation was less consistent across doses, with analgesia occurring in 100% of blocks with the 2.5% concentration vs 67% for 1.25%, and 50% for 0.625%. Anesthesia also less frequent, occurring in 83% of blocks for the 2.5% 120K EDLA concentration, vs 22% for 1.25%, and 17% for 0.625%). Analgesia with or without anesthesia occurred in 100% vs 67% of subjects for 40K EDLA and 40K IDLA, respectively; anesthesia occurred at the same rates (100% vs 67% for 40K EDKA and 40K IDLA, respectively).

Onset and Duration of Temperature Perception Block

In evaluating the Block of temperature perception (somesthetic test—onset and duration), temperature perception was assessed by a perceived change in temperature when the sensory blocked areas were touched with an alcohol swab. Subjects were instructed to answer “yes” if a change in temperature was felt, or “no” if no change was perceived. The answers were converted to a scale of 0-1, with 1=“no”, and 0=“yes”. Blocking of temperature perception was evaluated once every 12 hours until the offset of block, and once every 24 hours thereafter for a total of 14 days, regardless of offset.

Onset was defined as the first time at which the subject had not felt a change in temperature. Offset was defined as a return to baseline values for the somesthetic test. As shown in Table G6-Part 1, across doses, onset of temperature perception block was earlier for 40K EDLA than for 120K EDLA (80% of blocks set up at or before hour 6) compared to that observed for 120K EDLA (48% set up later than 6 hours, and 48% failed to set up). Onset of temperature perception block was earlier for 1.25% 40K EDLA compared to 1.25% 120K EDLA (≦6 h in 100% for 40K EDLA vs 11% for 120K EDLA. Both formulations had later onset than AB, which set up in <1 hour for 100% of blocks (see FIG. G4).

Onset of temperature perception ≦3 hours was observed more reliably with higher concentrations of 40K EDLA (0.625%=67% , 1.25%=67%, 2.5%=and 67%). No blockade of temperature perception was observed within 3 hours for the lowest concentration (0.312%) of 40K EDLA or for any concentration of 120K EDLA (see Table G6-Part 1).

Results for temperature perception block were consistent with those for analgesia; onset of block ≦1 hour occurred more reliably for 40K EDLA vs 40K IDLA (40K EDLA=33%, and 40K IDLA=0% (Table G6-Part 2). As shown in FIG. G5, the mean scores for temperature perception block showed 1.25% 40K EDLA set up faster than 1.25% 40K IDLA and lasted longer.

TABLE G6
Onset^{a} of Temperature Perception Block
Study Part 1

| | 120K EDLA | AB | 120K EDLA | AB | 120K EDLA | AB |
|---|---|---|---|---|---|---|
| Time to Onset of | 0.625% | 0.5% | 1.25% | 0.5% | 2.5% | 0.5% |

| | | | |
|---|---|---|---|
| Temperature Perception Block | N = 6 | N = 9 | N = 6 |

Number (%) Subjects

| | | | | | | |
|---|---|---|---|---|---|---|
| ≦30 min | 0 | 3 (50%) | 1 (11%) | 8 (89%) | 0 | 6 (100%) |
| >30 min ≦ 1 h | 0 | 3 (50%) | 0 | 0 | 0 | 0 |
| >1 ≦ 2 h | 0 | 0 | 0 | 0 | 0 | 0 |
| >2 ≦ 3 h | 0 | 0 | 0 | 0 | 0 | 0 |
| >3 ≦ 6 h | 0 | 0 | 0 | 0 | 0 | 0 |
| >6 h | 3 (50%) | 0 | 2 (22%) | 1 (11%) | 5 (83%) | 0 |

Study Part 1

| | | | | | | | | |
|---|---|---|---|---|---|---|---|---|
| | 40K EDLA | AB | 40K EDLA | AB | 40K EDLA | AB | 40K EDLA | AB |
| Time to Onset of | 0.312% | 0.5% | 0.625% | 0.5% | 1.25% | 0.5% | 2.5% | 0.5% |

| | | | | |
|---|---|---|---|---|
| Temperature Perception Block | N = 3 | N = 6 | N = 3 | N = 3 |

Number (%) Subjects

| | | | | | | | | |
|---|---|---|---|---|---|---|---|---|
| ≦30 min | 0 | 1 (33%) | 1 (17%) | 2 (33%) | 0 | 2 (67%) | 0 | 1 (33%) |
| >30 min ≦ 1 h | 0 | 2 (67%) | 0 | 4 (67%) | 1 (33%) | 1 (33%) | 0 | 2 (67%) |
| >1 ≦ 2 h | 0 | 0 | 2 (33%) | 0 | 0 | 0 | 0 | 0 |
| >2 ≦ 3 h | 0 | 0 | 1 (17%) | 0 | 1 (33%) | 0 | 2 (67%) | 0 |
| >3 ≦ 6 h | 1 (33%) | 0 | 1 (17%) | 0 | 1 (33%) | 0 | 1 (33%) | 0 |
| >6 h | 2 (67%) | 0 | 1 (17%) | 0 | 0 | 0 | 0 | 0 |

Study Part 2

| | | | | |
|---|---|---|---|---|
| | 120K EDLA | 120K IDLA | 40K EDLA | 40K IDLA |
| Time to Onset of | 1.25% | 1.25% | 1.25% | 1.25% |
| Temperature Perception Block | N = 3 | N = 3 | N = 6^{b} | N = 6 |

Number (%) Subjects

| | | | | |
|---|---|---|---|---|
| ≦30 min | 0 | 0 | 1 (17%) | 0 |
| >30min ≦ 1 h | 0 | 0 | 1 (17%) | 0 |
| >1 ≦ 2 h | 1 (33%) | 0 | 1 (17%) | 1 (17%) |
| >2 ≦ 3 h | 0 | 0 | 1 (17%) | 1 (17%) |
| >3 ≦ 6 h | 0 | 0 | 1 (17%) | 2 (33%) |
| >6 h | 1 (33%) | 0 | 1 (17%) | 0 |

^{a}Refers to only those subjects who had successful sensory blocks.
^{b}The 40K EDLA and 40K IDLA groups comprised the same 6 subjects who received EDLA in one foot and IDLA in the other.

In evaluating Duration of Temperature Perception Block, duration of temperature perception block was defined as the time between onset of block in response to cold and the time when there was a return of a sensation of cold. The results of the duration of Temperature Perception Block are summarized in Table G7. Across doses, duration of temperature perception block was longer for 40K EDLA compared to AB (3.2 days compared to 0.5 days, respectively), and compared to 120K EDLA (1.4 days). The duration of temperature perception block was longer for 40K EDLA compared to 120K EDLA (40K EDLA=3.2 days; 120K EDLA=1.4 days). Duration of temperature perception block was longer for higher vs lower concentrations of 40K EDLA (0.312%=1.2 days; 0.625%=2.3 224 days; 1.25%=3.2 days; 2.5%=4.2 days). The mean duration was longer for 40K EDLA compared to 40K IDLA (1.4 days vs 0.5 days, respectively). Duration of block for 40K IDLA was similar to that observed for AB (0.5 days vs 0.2-0.8 days for AB).

TABLE G7
Duration^{a} of Temperature Perception Block
Study Part 1

| | Treatment Pair | | Treatment Pair | | Treatment Pair | |
|---|---|---|---|---|---|---|
| | 120K EDLA | AB | 120K EDLA | AB | 120K EDLA | AB |
| | 0.625% | 0.5% | 1.25% | 0.5% | 2.5% | 0.5% |
| | N = 6 | | N = 9 | | N = 6 | |
| Duration (days) | | | | | | |
| Mean | 1.9 | 0.4 | 1.4 | 1.0 | 1.2 | 0.4 |
| (SE) | (1.2) | (0.1) | (0.7) | (0.6) | (0.5) | (0.1) |
| Range | 0.7, 4.2 | 0.1, 0.7 | 0.3, 2.8 | 0.2, 5.8 | 0.0, 2.4 | 0.0, 0.8 |

Study Part 1

| | Treatment Pair | | Treatment Pair | | Treatment Pair | | Treatment Pair | |
|---|---|---|---|---|---|---|---|---|
| | 40K EDLA | AB | 40K EDLA | AB | 40K EDLA | AB | 40K EDLA | AB |
| | 0.312% | 0.5% | 0.625% | 0.5% | 1.25% | 0.5% | 2.5% | 0.5% |
| | N = 3 | | N = 6 | | N = 3 | | N = 3 | |
| Duration (days) | | | | | | | | |
| Mean | 1.2 | 0.3 | 2.3 | 0.5 | 3.2 | 0.4 | 4.2 | 0.8 |
| (SE) | (0.5) | (0.1) | (0.2) | (0.1) | (0.9) | (0.2) | (0.6) | (0.03) |
| Range | 0.3, 1.9 | 0.1, 0.4 | 1.7, 2.7 | 0.2, 0.7 | 2.1, 5.0 | 0.2, 0.7 | 2.9, 4.9 | 0.7, 0.8 |

Study Part 2

Treatment Pair

| | 120K EDLA | 120K IDLA | 40K EDLA | 40K IDLA |
|---|---|---|---|---|
| | 1.25% | 1.25% | 1.25% | 1.25% |
| | N = 3 | N = 3 | N = 6^{b} | |
| Duration (days) | | | | |
| Mean | 0.9 | 0 | 1.4 | 0.5 |
| (SE) | (0.8) | 0 | (0.5) | (0.2) |
| Range | 0.1, 1.7 | 0 | 0, 3.2 | 0.1, 0.8 |

^{a}Refers to only those subjects who had successful temperature perception blocks.
^{b}The 40K EDLA and 40K IDLA groups comprised the same 6 subjects who received EDLA in one foot and IDLA in the other.

Degree of Numbness

Numbness was evaluated by the parameter, Degree of numbness, defined as the distribution of numbness ratings at each time point, and was based on an 11-point verbal rating scale; 0=not numb at all and 10=totally numb. Subjects were asked to rate their degree of numbness following touch to the sensory blocked areas on the top of the foot. Degree of numbness was evaluated once every 12 hours until the offset of block, and once every 24 hours thereafter for a total of 14 days, regardless of offset.

As shown in FIG. G6, the 40K formulation of EDLA demonstrated a greater degree of numbness compared to 120K EDLA. Across 40K EDLA doses, the mean peak numbness score post-injection was 8.2, compared to 2.4 for 120K EDLA. Mean peak numbness occurred at 12 hours following injection for both 40K EDLA and 120K EDLA. The comparable level of numbness for AB (mean score, 8.6) occurred 2 hours post-injection.

Within the 40K EDLA group, the highest mean numbness scores and the time of peak numbness for each concentration were as follows: 0.312%=7.7 at Hour 12; 0.625%=7.5 at Hour 12; 1.25%=10 at Hour 6; and 2.5%=9.3 on Day 2. Within the 120K EDLA group, the highest mean numbness scores and the time of peak numbness for each concentration were as follows: 0.625%=1.8 on Day 2; 1.25%=2.8 at Hour 12; 2.5%=3.7 on Day 3. The peak numbness scores for 40K EDLA and 40K IDLA were markedly different. The mean peak numbness score for 1.25% 40K EDLA was 9 and occurred on Day 2, compared to 4.8 for 40K IDLA, which occurred 6 hours post-injection (FIG. G7).

Number (%) of Unsuccessful Sensory Blocks

In evaluating Percent of unsuccessful sensory blocks, the percent of assessments in which neither anesthesia nor analgesia were demonstrated was calculated for each standardized test, by time-point. Across doses (Parts 1 and 2), the rate of unsuccessful blocks was 0% 40K EDLA and AB, compared to 38% of blocks for 120K EDLA. The rate of unsuccessful blocks was lower for 40K EDLA than for 40K IDLA (0% vs 33%, respectively). All (100%) of blocks for 120K EDLA and 120K IDLA were unsuccessful, as shown in Table G8).

TABLE G8
Number and Percent of Unsuccessful Sensory Blocks^{a}
Study Part 1 - 40K EDLA/AB

| Treatment Pair | | Treatment Pair | | Treatment Pair | |
|---|---|---|---|---|---|
| 120K | AB | 120K | AB | 120K | AB |
| EDLA | 0.5% | EDLA | 0.5% | EDLA | 0.5% |
| 0.625% | | 1.25% | | 2.5% | |

| | | |
|---|---|---|
| N = 6 | N = 9 | N = 6 |

Number (%) Subjects without Analgesia

| | | | | | |
|---|---|---|---|---|---|
| 3 (50%) | 0 | 3 (33%) | 0 | 0 | 0 |

Study Part 1 - 120K EDLA/AB

| Treatment Pair | | Treatment Pair | | Treatment Pair | | | |
|---|---|---|---|---|---|---|---|
| 40K EDLA | AB | 40K EDLA | AB | 40K EDLA | AB | 40K EDLA | AB |
| 0.312% | 0.5% | 0.625% | 0.5% | 1.25% | 0.5% | 2.5% | 0.5% |

| | | | |
|---|---|---|---|
| N = 3 | N = 6 | N = 3 | N = 3 |

Number (%) Subjects without Analgesia

| | | | | | | | |
|---|---|---|---|---|---|---|---|
| 0 | 0 | 0 | 0 | 0 | 0 | 0 | 0 |

Study Part 2 EDLA/IDLA

| | | Treatment Pair | |
|---|---|---|---|
| 120K EDLA | 120K IDLA | 40K EDLA | 40K IDLA |
| 1.25% | 1.25% | 1.25% | 1.25% |
| N = 3 | N = 3 | N = 6^{b} | N = 6 |

Number (%) Subjects without Analgesia

| | | | |
|---|---|---|---|
| 3 (100%) | 3 (100%) | 0 | 2 (33%) |

^{a}Subjects felt 2 or 3 of the 3 of the pinpricks as sharp (unsuccessful sensory block)
^{b}The 40K EDLA and 40K IDLA groups comprised the same 6 subjects who received EDLA in one foot and IDLA in the other.

Pharmacokinetics/Pharmacodynamic Meausures

In evaluating Pharmacokinetics/Pharmacodynamic Measures, subjects had blood drawn pre-dose (baseline), 3 and 6 hours post-injection and approximately every 24 hours until the offset of the block was determined. Plasma bupivacaine and dexamethasone concentrations over time were determined for 120K EDLA and 120K IDLA. Dexamethasone and bupivacaine concentrations were determined using liquid chromatography with MS-MS detection technique. The calibration ranged from 50.0 to 6400 pg/mL for dexamethasone and 5.00 to 640 ng/mL for bupivacaine, where the limit of quantitation (LOQ) was 50.0 pg/mL for dexamethasone and 5.00 ng/mL for bupivacaine.

The results showed plasma bupivacaine and dexamethasone concentrations were below the limits of detection for the assay (5.0 ng/mL for bupivacaine and 50.0 pg/mL for dexamethasone, data not shown).

Pain on Injection.

Mean pain on injection scores are presented in Table G9. With respect to pain on injection scores, the 120K EDLA groups appeared to experience slightly more pain upon injection when compared with AB and 120K IDLA (average scores ranging from 3.8 to 5.7 for 120K EDLA; 3.8 to 4.7 for AB; 2.7 was the average score for the 120K IDLA group). This effect was not evident in the 40K EDLA groups versus their active controls or versus 40K IDLA.

TABLE G9
Pain on Injection Scores^{a} (Mean (+/−SE))
Study Part 1

| 120K EDLA | AB | 120K EDLA | AB | 120K EDLA | AB |
|---|---|---|---|---|---|
| 0.625% | 0.5% | 1.25% | 0.5% | 2.5% | 0.5% |

| | | |
|---|---|---|
| N = 6 | N = 9 | N = 6 |

Pain on injection (mean [SE])

| | | | | | |
|---|---|---|---|---|---|
| 5.7 (0.6) | 3.8 (0.5) | 5.1 (0.9) | 4.7 (0.8) | 4.6 (1.2) | 3.8 (1.2) |

Study Part 1

| | | | | | | | |
|---|---|---|---|---|---|---|---|
| 40K EDLA | AB | 40K EDLA | AB 0.5% | 40K EDLA | AB 0.5% | 40K EDLA | AB 0.5% |
| 0.312% | 0.5% | 0.625% | | 1.25% | | 2.5% | |

| | | | |
|---|---|---|---|
| N = 3 | N = 6 | N = 3 | N = 3 |

Pain on injection (mean [SE])

| | | | | | | | |
|---|---|---|---|---|---|---|---|
| 2.3 (1.2) | 2.3 (0.9) | 3.7 (0.6) | 4.5 (0.8) | 3.0 (1.2) | 1.0 (1.0) | 0.7 (0.7) | 3.7 (0.3) |

Study Part 2

| | | | |
|---|---|---|---|
| 120K EDLA | 120K IDLA | 40K EDLA | 40K IDLA |
| 1.25% | 1.25% | 1.25% | 1.25% |
| N = 3 | N = 3 | N = 6^{b} | N = 6^{b} |

Pain on injection (mean [SE])

| | | | |
|---|---|---|---|
| 4.0 | 2.7 | 3.2 | 3.5 |
| (2.0) | (0.3) | (1.0) | (0.6) |

^{a}During each injection of the superficial peroneal nerve, the subject was asked to evaluate the pain of the injection (not needle insertion) using an 11-point verbal rating scale where 0 = no pain and 10 = pain as bad as you can imagine.
^{b}The 40K EDLA and 40K IDLA groups comprised the same 6 subjects who received EDLA in one foot and IDLA in the other.

Conclusions

In general, EDLA had a longer onset and duration of action than 0.5% AB, both in terms of analgesia (with or without anesthesia) and temperature perception block. Dexamethasone was effective in prolonging the action of EDLA; IDLA generally had a duration of action similar to AB. The 40K EDLA formulation, especially in concentrations of 1.25% and 2.5%, appeared to be a safe and effective method of producing local analgesia of extended duration.

### Example H

### The Efficacy of 40K EDLA Administered as a Peripheral Nerve Block for Post-operative Analgesia Following Podiatric Surgery

A double blind, randomized, dose-ranging study evaluated doses of 40 kilodaltons (K) Extended Duration Local Anesthetic (EDLA) to achieve an analgesic block post-operatively lasting three to five days. Each patient who participated in the study was scheduled to undergo unilateral podiatric surgery (bunionectomy with single osteotomy). Patients were administered a peripheral nerve block (Mayo Block) with 18 milliliters (mL) of aqueous bupivacaine 0.5% (90 milligrams (mg)) for surgical anesthesia. At the end of surgery, patients were randomized to receive an additional anesthetic block, using 18 mL of 40K EDLA 0.625%, 1.25%, or 2.5% (81 mg, 162 mg, or 324 mg bupivacaine respectively), or 18 mL of normal saline for injection (placebo). After surgery, all patients received a prescription for hydrocodone 5 mg/500 mg acetaminophen (APAP (Lortab)) to be taken every four hours as needed for post-operative pain relief.

The duration of the study was approximately 6 days (+/−1 day). Follow-up evaluations were required at 14 days (+/−2 days), three months (+/−2 weeks) and a long-term follow up at six months (+/−2 weeks) post surgery. In addition, the patient was contacted by telephone every day from day 1 until the day 6 evaluation and again at 6 weeks (+/−1 week) post-surgery.

A specified amount of diluent was added to the vials containing 100 mg 40K EDLA microsphere (72% by weight of bupivacaine and 0.04% dexamethasone) powder to yield a specific microsphere concentration, as shown in Table H1 below:

TABLE H1

| mL of Diluent Added | Concentration (%) | Microspheres (mg/mL) |
|---|---|---|
| 16 | 0.625 | 6.2 |
| 8 | 1.25 | 12.5 |
| 4 | 2.5 | 25.0 |

Baseline evaluations/procedures that were performed on the same day and prior to the patient's surgery included baseline pain score (on a 0-10 scale) and degree of numbness assessments.

Prior to podiatric surgery (bunionectomy with single osteotomy), all patients were administered a peripheral nerve block (Mayo Block) with 18 mL of 0.5% aqueous bupivacaine for surgical anesthesia. Additional 0.5% aqueous bupivacaine, up to 10 mL, were administered intra-operatively for additional anesthesia if necessary.

After wound closure, the patients were administered an additional block using 18 mL of Normal Saline for Injection as a control or 18 mL 40K EDLA 0.625%, 1.25%, or 2.5%. The post-operative Mayo Block was administered in the same manner and technique as the pre-operative Mayo Block.

After surgery, all patients received instructions on standard post-operative care, as well as instructions on recording results of efficacy evaluations. The evaluator performed the initial 1-hour post-operative evaluations. With the evaluator's help, the patient performed the second evaluation at the time of discharge and recorded the results.

Patients were instructed to complete the evaluations every day up until the post-operative visit on Day 6. The patient performed the pain scores based on a 0-10 scale twice a day; once upon awakening and again prior to going to bed with additional pain scores performed prior to taking rescue pain medication (including waking up at night due to pain—“Quality of Sleep”). They also recorded Quality of Pain Assessment (the type of pain over the last 24 hours) prior to going to bed every evening and recorded the number of tablets of rescue pain medication taken and the times it was taken. All patients were required to return to the site at 6 days post-surgery for post-operative pain assessments (Pain Score and Quality of Pain) and the degree of numbness.

Efficacy

Evaluation/procedures included Pain Scores, Pain Intensity (Quality of Pain), Quality of Sleep, Degree of Numbness, and Rescue pain Medication

Pain Scores

Pain Scores based on a 0-10 scale were measured twice a day (morning and evening), and prior to taking rescue pain medication. The patient was asked to assess the degree of post-operative pain by looking at a horizontal 11 point scale and circling the number that best described his/her pain (0=no pain, and 10=pain as bad as can be imagined).

The mean daily pain scores showed that there were differences between treatment with 1.25% and 2.5% EDLA concentrations in comparison with placebo. These differences were most evident in the first two days post-operatively.

Time to First Pain

Patients were instructed to record the time to first pain greater than 3, using the 0-10 scale discussed above. EDLA at 1.25% and 2.5% showed efficacy in human patients, delaying the. perception of pain for at least 1 day. As shown in FIG. H1, the formulations also exhibited a dose response relationship for median time to first pain >3 for EDLA at 1.25% and 2.5% of approximately 21 hours and 43 hours, respectively.

Pain Intensity (Quality of Pain)

Pain Intensity (Quality of Pain) was measured at the end of each day. The patient was asked to assess the quality of pain he/she experienced during the past 24 hours by rating the pain level with each of the following adjectives on a scale of 0-3 (0=none; 1=mild; 2=moderate; and 3=severe throbbing, shooting, stabbing, sharp, cramping, gnawing, hot-burning, aching, heavy, tender, splitting, tiring-exhausting, sickening, fearful, and punishing-cruel.

Quality of Sleep

Quality of Sleep was measured. The patient was asked to record the number of times he/she awoke during the night to take rescue pain medication. If the patient awoke due to pain, he/she recorded the time, pain score (0-10 scale) assessment, and the number of tablets of rescue pain medication taken. There was no significant difference between treatments in the number of night awakenings due to pain.

Degree of Numbness

Degree of Numbness (to the touch) was measured twice a day (morning and evening). The patient was asked to determine the degree of numbness of the blocked area. The patient was asked to touch the area on top of the operative foot using the index finger and rate the degree numbness based on an 11 point scale (0=not numb at all; 10=totally numb).

Rescue Pain Medication

Rescue Pain Medication was measured. All patients received a prescription for 40 tablets of Lortab to be taken for post-operative breakthrough pain. The patient was instructed to take 1-2 tablets every 4 hours as needed—only when post-operative pain becomes uncomfortable, not in anticipation of pain. The patient was instructed to record the level of pain (pain scores based on a 0-10 scale) prior to taking the medication and to record the pain score together with time and number of tablets taken.

The total rescue dose (total number of rescue tablets used) was statistically different for all doses of EDLA in comparison with placebo, with the 2.5% EDLA formulation showing greatest efficacy. The time to first use of rescue medication also demonstrates efficacy. As shown in FIG. H2, a dose response relationship for the time to first use of rescue was observed, with the rank order of efficacy being 2.5%>1.25%>0.625% EDLA>placebo.

### Example I

### Safety Evaluations

Studies were performed to assess the potential for Extended Duration Local Anesthetic (EDLA) to cause tissue irritation as well as to assess the safety and etiology of delayed onset swelling/induration following subcutaneous injection. The formulations were shown to be safe.

The injected medications used in these studies included 120K EDLA at concentrations of 1.25%, 2.5% and 5.0%, 120K IDLA at concentration of 1.25%, and aqueous bupivacaine at concentrations of 0.25% and 0.5%. These studies also included two placebo injections: 1) empty microspheres (concentration of 1.25%) suspended in diluent (without bupivacaine) to separate out any effects due to the microspheres themselves, and 2) injections with only the diluent to study the effects of the diluent.

All injections (0.2 or 6 milliliters (mL)) were administered subcutaneously to the volar surfaces of the arm. The actual dose of active drugs in milligrams (mg) administered per mL is shown in Table I1 below.

TABLE I1
Doses of Active Drugs

| INJECTION | ACTIVE DRUG ADMINISTERED PER mL |
|---|---|
| 120K EDLA 1.25% | 9.38 mg bupivacaine |
| 120K EDLA 2.5% | 18.75 mg bupivacaine |
| 120K EDLA 5.0% | 37.50 mg bupivacaine |
| 120K IDLA 1.25% | 9.38 mg bupivacaine |
| Aqueous Bupivacaine | 2.5 mg bupivacaine |
| 0.25% | |
| Aqueous Bupivacaine 0.5% | 5.0 mg bupivacaine |

*The two placebo injections (Empty Microspheres 1.25% and diluent only) contained no bupivacaine.

Safety evaluations included Adverse Events and Localized Response to Injections.

Safety Evaluation

1. Summary of Adverse Events

Six subjects (37.5%) reported 8 adverse events of which 6 were considered to be study medication related. The most common adverse events were itching and burning that were defined as pruritus and pain, respectively. There were 2 adverse events that were not related to medication.

The adverse events of pain (burn) was reported by 2 subjects (Nos. 1,13) and included sites that received empty microspheres, and 0.25% and 0.5% EDLA. Pruritus was observed in Subject No. 15 and appeared at 5 of the 7 sites, including the 3 EDLA concentrations, aqueous bupivacaine 0.5%, and diluent sites.

No clinically significant changes in vital signs were recorded at any time during the studies. There were no serious adverse events or deaths.

Localized Response to Infection

Pain on injection as measured in this study did not result in a clear differentiation between treatments. However, the 3 EDLA concentrations provided the highest percent of sites with a “no pain” category, 15 of the 16 subjects (87.5%) with this response compared to 3 of 16 subjects (18.8%) for 0.5% Aq. Bupivacaine.

No pattern was observed for skin color change which was variably recorded as ranging from 25% to 64% at the 7 injection sites. A return to normal for 80% or more of the sites was recorded by 24 to 36 hours. The area of skin color (in mm) did not differ between treatment sites with an initial area ranging from 10 to 14 mm among the 7 test sites. The area of discoloration then noticeably diminished by 3 hours to a range from 2 to 6 mm.

Elevation of a wheal at the injection site was noted as “raised” at most of the sites at 15 minutes after injection, which then resolved to none for the great majority at 3 to 6 hours. The incidence of elevations was not as frequent for the 2 aqueous bupivacaine injections, although the diluent alone was raised in 81.3% of the sites. This effect is most likely due to the physiological properties of bupivacaine. The empty microspheres and the 3 EDLA concentrations were raised in 75% or more of their test sites.

Localized reactions of itching, burning, swelling, etc. were infrequent with 3 reports of burning and 3 reports of itching. The 3 burn sensations were reported for empty microspheres and 2.5% and 5.0% EDLA. The 3 itching responses were reported for diluent and twice for the 1.25% EDLA.

The above summary provides a benign safety profile for the EDLA injections at these concentrations in a tissue irritation study. The response to EDLA was not different from that seen for diluent, empty microspheres or the 2 aqueous bupivacaine concentrations. The results indicate no dermal or intradermal tissue irritation reactions to EDLA injections at these volumes and concentrations.

Special Safety variables assessed delayed onset swelling/induration, which was defined as induration or swelling which occurred within or near the site of injection, approximately 5 or more days following injection, and following resolution of any immediate post-injection swelling due to drug volume and/or needle (mechanical) irritation. All injection sites that developed delayed onset swelling/induration were biopsied and the tissue examined histologically. A total of 10 biopsies were performed, 5 of which were bilateral.

Results of Histological Examination

Tissue Reaction

Tissue/cellular reactions (e.g., abscess, fibrosis, necrosis, and neovascularization) were characterized by classifying and quantifying the cells (e.g., eosinophils, fibroblasts, foreign body giant cells, lymphocytes, macrophages, monocytes, and polymorphonuclear leukocytes) found at the tissue/microsphere interface and rated as 0 (none), 1 (minimal), 2 (mild), 3 (moderate), or 4 (extensive).

Biopsies from all 3 treatments resulted in significant tissue reaction. Fifty percent of placebo biopsies had at least some tissue reaction. Biopsies from the IDLA injection sites had the greatest percentage of samples showing some (minimal, mild or moderate) tissue reaction (64%), followed by biopsies from EDLA injection sites (58%), and those from placebo injection sites (50%).

The greatest treatment-specific differences found in the cell types present in the biopsies were in the presence of fibroblasts, macrophages and polymorphonuclear leukocytes. Eighty percent of EDLA biopsies received a ‘minimal’ rating for fibroblasts but all of the IDLA samples received ‘minimal’ ratings and only one (50%) of the placebo biopsies was rated ‘minimal’. For macrophages, all three IDLA biopsies were rated either ‘mild’ (33%) or ‘moderate’ (67%), while 40% of EDLA samples and 50% of placebo samples were rated either ‘mild’ or ‘moderate’. Forty percent of EDLA samples were rated minimal or mild for the presence of polymorphonuclear leukocytes but all of the placebo biopsies were rated ‘none’.

Biopsies from all 3 treatments had similar ratings for abscess and necrosis. Ratings for fibrosis were similar to those observed for fibroblasts: 80% of EDLA biopsies were rated minimal, 100% of IDLA samples were rated minimal and only 50% of placebo samples were rated minimal. EDLA and IDLA biopsies also showed slightly more neovascularization than did placebo samples.

Microsphere Disposition and Degradation

In evaluating Microsphere Disposition/Biodegradation, microspheres were marked as present or not. If microspheres were present the extent of microsphere biodegradation was rated as 1) no visually apparent degradation, 2) partial degradation, or 3) extensive degradation.

Microspheres were present in all biopsies with the exception of one EDLA sample. The two placebo biopsies showed no apparent degradation of polymer particles while all of the microspheres in samples from IDLA and EDLA injection sites showed partial degradation.

Overall Character of the Tissue Reaction

The etiologic profile of the tissue reaction was selected from among the following catagories: 1) acute inflammation, 2) chronic inflammation, 3) granulation tissue, 4) foreign body reaction, 5) fibrosis, 6) fibrous capsule, 7) infection, and 8) drug/chemical reaction.

The tissue reaction location was indicated as either focal (within the microsphere implant site) or diffuse (outside of the microsphere implant site). A five-point rating scale from 0 (none) to 4 (extensive) was again used to score the reaction. Although biopsies from IDLA-treated injection sites had the greatest overall tissue reaction, only EDLA samples showed tissue reaction outside of the microsphere implant site. Three EDLA biopsies had diffuse chronic inflammation and 3 had diffuse drug/chemical toxic reaction. No other biopsies, from any treatment, had tissue reaction outside the microsphere implant site (diffuse). Acute inflammation, granulation tissue and infection were not observed in any of the biopsies. With the exception of 1 EDLA biopsy, all biopsies showed chronic inflammation. Sixty percent of the EDLA samples had chronic inflammation outside the microsphere implant site (diffuse). Chronic inflammation within the microsphere implant site (focal) was observed for all of the IDLA and placebo biopsies and 20% of EDLA samples. The assessments for drug/chemical toxic reactions were the same as for chronic inflammation: 60% of EDLA samples showed diffuse location and all of the IDLA and placebo samples showed focal location. A single IDLA biopsy was the only biopsy to have a fibrous capsule. Fibrosis and foreign body reactions, when present, were observed only within the microsphere implant site.

Overall Assessment Rating

Overall, biopsies from EDLA injection sites showed the least tissue reaction, followed by biopsies from placebo injection sites and biopsies from IDLA injection sites. None of the biopsies were assessed to have granulation tissue or infection. One biopsy from an EDLA injection site was assessed a mild rating for acute inflammation but all other biopsies were rated as having no acute inflammation. Two of the EDLA biopsies (40%) were rated as having moderate chronic inflammation and one (20%) showed no chronic inflammation. The remaining 2 EDLA biopsies were rated as having minimal or mild chronic inflammation, as were all of the IDLA and Placebo biopsies. One (33%) of the IDLA samples was rated minimal for fibrous capsules and all other IDLA, as well as all EDLA and placebo, biopsies were rated as having none.

Biopsies from all three treatment injection sites showed some foreign body reaction. One EDLA (20%), 2 IDLA (67%) and 1 placebo (50%) biopsy were rated moderate for foreign body reaction. One EDLA sample (20%) was rated as having none and the remaining biopsies were rated as having minimal foreign body reaction. Only a single EDLA biopsy (20%) was rated as having no drug/chemical toxic reaction. All other biopsies had either minimal or mild drug/chemical toxic reaction.

Site with More Significant Tissue Reaction

None of the IDLA/placebo treatment pairs were biopsied, therefore the only treatment pairs that were compared were EDLA/placebo and EDLA/IDLA. When these treatment pairs were analyzed for more significant tissue, no difference was noted in 51.5% of the categories for the EDLA/placebo pairs and in 54.5% of the categories for EDLA/IDLA pairs. However, when a difference was noted, the EDLA biopsies were more often selected over the placebo pairs (36.4% versus 9.1%), and the IDLA biopsies were more often selected over the EDLA biopsies (33.3% versus 15.2%). The greatest single difference noted for the EDLA/placebo pairs was in the comparison of neovascularization: the EDLA biopsy was selected as the site with more significant neovascularization both times. Most notably for the EDLA/IDLA treatment pairs, 2 of 3 (67%) IDLA biopsies were selected as having more significant presence of foreign body giant cells, lymphocytes, macrophages and monocytes.

Site with More Significant Microsphere Degradation and Overall Reaction

Two of 2 (100%) EDLA biopsies were selected over their placebo pairs as having more significant degradation and 1 of the 3 (33%) IDLA biopsies was selected over its EDLA pair as having more significant microsphere degradation. The other 2 pairs showed no difference in microsphere degradation.

Most treatment pairs, when analyzed for overall reaction in 8 classifications, showed no difference. No difference was noted in 63% of EDLA/placebo pairs and in 54% of EDLA/IDLA pairs. When a difference between EDLA and placebo biopsies was apparent, EDLA was selected as having more overall reaction in 31% of the reaction classifications, and placebo was selected in only 6% of the cases. Both EDLA samples had more significant chronic inflammation than did their placebo partners. Among the EDLA/IDLA pairs, IDLA biopsies were selected 33% and EDLA 13% of the time, overall. IDLA biopsies were selected as demonstrating more significant overall chronic inflammation, drug/chemical toxic reaction, and foreign body reaction in 2 of 3 (67%) pairs.

Delayed Onset Swelling/Induration and Biopsy Evaluations: Delayed onset swelling/induration was observed at more than half (54 percent) of all injection sites but most frequently at EDLA injection sites. Eight of 10 (80%) EDLA injection sites showed delayed onset swelling/induration while only 5 of 9 (56%) IDLA injection sites and 2 of 9 (22%) placebo injection sites demonstrated delayed onset swelling/induration. The mean time to onset of swelling/induration was similar for all of the treatments, ranging from 28.5 to 30.1 days. The mean duration of delayed onset swelling/induration for placebo injection sites (70.4 days) was more than double the mean duration for EDLA (35.0 days) and IDLA (28.1 days) injection sites. The range of duration of swelling/induration was equally broad for EDLA (7-64 days) and IDLA (7-60) injection sites but much tighter for placebo injection sites (68-72 days). The mean for the maximal area of swelling/induration was similar for all treatments.

Conclusions

Most adverse events were site-specific and were expected with this formulation. Most were mild and resolved without intervention. The relative absence of systemic adverse events suggested a safety profile characterized by minimal plasma bupivacaine concentrations. There were no systemic adverse events at the 6-month follow-up and none of the adverse events at 6-month follow up was serious or severe.

Fifty four percent of injection sites developed the delayed onset swelling/induration that this study was principally designed to evaluate. Eight of these received EDLA, 5 received IDLA and 2 received placebo. The mean time to delayed onset swelling/induration was similar for the 3 treatments as was the mean maximal area of swelling/induration.

Five subjects demonstrating bilateral delayed onset swelling/induration were biopsied, yielding 10 biopsies. Five of the biopsies were from injection sites that had been administered EDLA, 3 IDLA and 2 placebo. All biopsies exhibited significant tissue reaction although IDLA biopsies exhibited the most and placebo biopsies the least. In the second part of the biopsy evaluation biopsy pairs were reunited and comparatively evaluated. When EDLA/IDLA biopsies were comparatively evaluated, biopsies from IDLA administration sites were most often selected as having the more significant tissue and overall reaction. When EDLA/placebo biopsies were evaluated, biopsies from EDLA administration sites were most often selected. Although EDLA treatment resulted in the most delayed onset swelling/induration and biopsies from subjects who received IDLA demonstrated the most tissue reaction, a significant fraction of subjects who received placebo also developed delayed onset swelling/induration.

### Example J

### Microdialysis Study

DURAIN®, also referred to as 40K EDLA (Extended Duration Local Analgesic), is being investigated as a means of providing long-acting local analgesia for the management of acute post-operative/procedural pain by blockade of small nerve endings via infiltration, and by blockade of selected peripheral nerves by perineural administration.

The product combines bupivacaine free base, a local anesthetic of the amide class, and dexamethasone, a synthetic adrenocorticoid included in DURAIN® solely for its observed ability to prolong the duration of action of bupivacaine. The two active ingredients are encapsulated in a slightly porous shell composed of polylactic-co-glycolic acid polymers (MW=40 kD) in a 65:35 ratio. 120K EDLA, a previously studied formulation, differed from DURAIN® in that it employed polymer with a molecular weight of 120 kD. Bupivacaine (free base) comprises approximately 72% of total microcapsule mass and dexamethasone comprises 0.04%. Bupivacaine-loaded microspheres without dexamethasone are referred to as 40K IDLA (Intermediate Duration Local Analgesic). In both products, the sterile, ingredient-loaded microcapsules are formed into a dry powder for storage and shipment. When suspended in a specialized aqueous diluent, they form a fine suspension, suitable for injection.

After administration of DURAIN®, the active ingredients diffuse slowly from the microcapsules, which ultimately biodegrade at the injection site. Onset of effect (within 30 to 60 minutes) is significantly later than that observed with aqueous forms of local anesthetics, while duration of effect is significantly longer (up to 5 days). Because of a later onset, longer duration and (intended) reduced density of block, DURAIN® is suitable for analgesia rather than anesthesia.

Microdialysis was used in the evaluation of the subcutaneous administration of 40K EDLA and Aqueous Bupivacaine to determine the correlation of subcutaneous tissue and plasma bupivacaine and dexamethasone concentrations with local sensory testing. The study was used to determine the feasibility of using microdialysis technique to study the relationship between local tissue concentrations of bupivacaine and dexamethasone from 40K EDLA and local sensory response. In addition, the relationship between local tissue concentrations and plasma concentrations of the drugs. The study also investigated the feasibility of using non-invasive MRI methods to monitor 40K EDLA microsphere disposition over time. The study also explored the feasibility of using LASER Doppler to evaluate the effect of 40K EDLA on local blood flow. Local anesthetics generally cause cutaneous vasodilation. Measurement of skin blood flow velocity by LASER Doppler was evaluated as a measure of the duration of effect of 40K EDLA.

A 2-part trial was conducted at a single site. Part 1 was an open-label, randomized parallel group design in normal, healthy, young adult, male and female subjects, conducted to assess the relationship between tissue and plasma bupivacaine/dexamethasone concentrations and local sensory response. Three groups of 4 subjects each were randomly assigned to receive subcutaneous infiltration of 1 of the following 3 treatments: 15 mL 40K EDLA 2.5% unilaterally in the left calf; 15 mL 40K EDLA 2.5% bilaterally in both calves; or 15 mL aqueous bupivacaine 0.5% bilaterally in both calves. Plasma and local tissue concentrations of the drug were to be evaluated and sensory testing performed at specific intervals on the day of injection, and daily for 3 additional days. MRI scanning was used to assess residence time and local tissue response to microspheres, and LASER Doppler evaluation was used to assess local blood flow at specified time points. MRI scanning would be conducted only on subjects randomized to receive 40K EDLA 2.5%. The core period of Part 1 of the study (Days 1-4) was completed prior to the start of Part 2.

Part 2 was a double-blind (subject/evaluator), randomized, parallel group trial to explore the relationship between dose (volume and concentration) of 40K EDLA and sensory response. Two groups of 6 subjects each received either 7.5 mL 40K EDLA 2.5% in the right calf and 15 mL 40K EDLA 2.5% in the left calf, or 15 mL 40K EDLA 1.25% in the right calf and 15 mL 40K EDLA 2.5% in the left calf. Four additional subjects were randomized to receive aqueous bupivacaine 0.25% bilaterally in both calves. Subjects in this part of the study underwent similar assessments at the same time points as in Part 1. Tissue and plasma drug concentrations were to be measured as in Part 1.

Subjects were assessed for 4 days for pharmacodynamic, pharmacokinetic, pharmacokinetic-pharmacodynamic, and safety variables. Safety follow-up evaluations were conducted at 2 weeks (±2 days), 6 weeks (±2 weeks), 3 months, and 6 months post-injection. The pharmacodynamic variables included sensory testing (pinprick testing, somesthetic testing, warmth detection threshold, and heat pain detection threshold), skin blood flow assessments using LASER Doppler evaluations, and MRI assessments. The primary pharmacokinetic variables included AUCt (area under the plasma or tissue concentration time course profile from dosing to last quantifiable concentration), AUC∞ (area under the plasma or tissue concentration time course profile from dosing to infinity), and Cmax (maximum observed plasma or tissue concentration). The pharmacokinetic-pharmacodynamic variables included correlation of subcutaneous tissue and plasma bupivacaine concentrations with local sensory response. The safety variables included clinical laboratory tests, medical histories, vital signs, physical examinations, electrocardiogram, and adverse events. The study design is summarized in FIG. J-1.

In Part 1 of the study, there were 3 different treatment groups:

The first treatment group, unilateral 15 mL 40K EDLA 2.5%, was chosen to determine the relationship between local tissue concentrations and plasma concentrations of bupivacaine and dexamethasone with 40K EDLA administration, as well as to determine the relationship of plasma bupivacaine concentrations to sensory testing and the relationship of tissue bupivacaine concentrations to sensory testing.

The second treatment group, bilateral 15 mL 40K EDLA 2.5%, was chosen to compare to the pharmacokinetic profile of the first treatment in order to establish the dose-proportionality for systemic exposure. In addition, the calf implanted with the microdialysis probe was to be used to determine the relationship between local tissue concentrations of 40K EDLA and sensory testing. The results of sensory testing from the calf implanted with the dialysis probe was to be compared to the results obtained from the opposite calf which had no dialysis probe in order to determine whether the presence of the probe affected sensory testing.

The rationale for the third treatment group, bilateral 15 mL aqueous bupivacaine 0.5%, was similar to that for bilateral 15 mL 40K EDLA 2.5%. The calf implanted with the microdialysis probe was to be used to determine the relationship between local tissue concentrations of bupivacaine and sensory testing. The results of sensory testing from the calf implanted with the dialysis probe was to be compared to the results obtained from the opposite calf which had no dialysis probe in order to determine whether the presence of the probe affected sensory testing.

For all 3 treatment groups in Part 1, LASER Doppler was used to assess changes in local blood flow to assess the duration of study medication effect. In addition, all 3 treatment groups in Part 1 were to be assessed to examine the effect of local pH changes on the local release of bupivacaine at the sites where the microdialysis probe was implanted. MRI was used to evaluate residence time and disposition of the microspheres over time only at sites where 40K EDLA 2.5% was injected.

In Part 2, there were 3 different treatment groups:

The first treatment group, 7.5 mL 40K EDLA 2.5% in the right calf and 15 mL 40K EDLA 2.5% in the left calf, was chosen to examine the effect of volume and dose of 40K EDLA (while holding concentration constant) on local tissue bupivacaine exposure and sensory testing.

The second treatment group, 15 mL 40K EDLA 1.25% in the right calf and 15 mL 40K EDLA 2.5% in the left calf, was chosen to examine the effect of dose and concentration of 40K EDLA (while holding volume constant) on local tissue bupivacaine exposure and sensory testing. The 15 mL 40K EDLA 1.25% treatment in this treatment group was also to be compared to the 7.5 mL 40K EDLA 2.5% in the first treatment group, in order to examine the effect of concentration and volume of 40K EDLA (while holding total dose constant).

The third treatment group, bilateral 15 mL aqueous bupivacaine 0.25%, was chosen as a control to compare to the plasma concentrations, and local blood flow evaluations of the other two treatment groups in Part 2.

For all 3 treatment groups in Part 2: plasma concentrations were to be determined for each bilateral combination of treatments; and local blood flow was to be assessed by LASER Doppler for each site.

The treatments administered in the study are indicated in Table J-1 below:

TABLE J-1

| | | |
|---|---|---|
| Treatment | Volume | Total dose |

| | | | |
|---|---|---|---|
| 40K EDLA 1.25% | 15 mL | Bupivacaine | 135.0 mg |
| | | Dexamethasone | 75.0 mcg |
| 40K EDLA 2.5% | 7.5 mL | Bupivacaine | 135.0 mg |
| | | Dexamethasone | 75.0 mcg |
| 40K EDLA 2.5% | 15 mL | Bupivacaine | 270.0 mg |
| | | Dexamethasone | 150.0 mcg |
| AB 0.25% | 15 mL | Bupivacaine | 37.5 mg |
| AB 0.5% | 15 mL | Bupivacaine | 75.0 mg |

The investigational drugs used in the study are listed in Table J-2 below:

TABLE J-2

| | 40K EDLA | AB 0.25% | AB 0.5% | Diluent |
|---|---|---|---|---|
| Dosage form | Suspension | Solution | Solution | Solution |
| Dose | 15 mL of | 15 mL of 0.25% | 15 mL of 0.5% | N/A |
| | 1.25%; | | | |
| | 7.5 mL and 15 | | | |
| | mL of 2.5% | | | |
| Ingredients | Bupivacaine, | Aqueous | Aqueous | Sodium |
| | dexamethasone | Bupivacaine | Bupivacaine | carboxymethylcellulose, |
| | | | | Polysorbate 80, |
| | | | | Mannitol, and |
| | | | | water for injection |
| Manufacturer | P. F. Labs | Abbott | Abbott | P. F. Labs |
| | | Pharmaceuticals | Pharmaceuticals | |
| Batch/Lot | CB27-35-VIF | 550653A | 531353A | 851-53-0002 |
| number | | | | |
| Site of | Totowa, NJ | Abbott | Abbott | Totowa, NJ |
| manufacture | | Pharmaceuticals | Pharmaceuticals | |

40K EDLA and diluent were supplied in labeled containers by the Purdue Frederick Company for PPLP. 40K EDLA was supplied in 10 mL vials containing 100 mg per vial of open-label medication and was stored at −5° Celsius (23° F.). Diluent was supplied in 30 mL vials of open-label medication and stored at controlled room temperature. AB (aqueous bupivacaine) was obtained commercially from Abbott Pharmaceuticals.

The schedule of visits and procedures is shown in Table J-3 below:

TABLE J-3

| | | Day 1 | Day 2 | Day 3 | Day 4 | Week 2 | Week 6 | Month 3 | Month 6 |
|---|---|---|---|---|---|---|---|---|---|
| | Screen | Visit | Visit | Visit | Visit | Visit | Visit | Call | Call |
| Informed Consent | X | | | | | | | | |
| Demographic | X | | | | | | | | |
| Medical History | X | | | | | | | | |
| Surgical History | X | | | | | | | | |
| Physical Exam | X | | | | X | | X | | |
| Vital Signs¹ | X | X | X | X | X | X | X | | |
| Laboratory | X | | | | X | | | | |
| ECG² | X | X | | | | | | | |
| Pregnancy Test | X | X | | | | | | | |
| Concomitant Meds | X | X | X | X | X | | X | X | X |
| Injection Site | | X | | | | | | | |
| Preparation³ | | | | | | | | | |
| Treatment | | X | | | | | | | |
| Assignment | | | | | | | | | |
| Administration of | | X | | | | | | | |
| Study Drug | | | | | | | | | |
| Probe Insertion⁴ | | X | | | | | | | |
| MRI Scanning | | Prior to inj. | | | X | X | X | | |
| | | and at 3 h | | | | | | | |
| | | post-inj. | | | | | | | |
| Sensory and | | Prior to inj., 30 | 48 h | 72 h | 96 h | | | | |
| LASER Doppler | | min., | | | | | | | |
| Testing | | 1, 3, 6, 12, 24 h | | | | | | | |
| | | post-inj. | | | | | | | |
| Dialysis Samples | | Prior to inj., | 48 h | 72 h | 96 h | | | | |
| | | 1-3 h, 6, 12, 24 | | | | | | | |
| | | h post-inj. | | | | | | | |
| Plasma Samples | | Prior to inj., 30 | 48 h | 72 h | 96 h | | | | |
| | | min., | | | | | | | |
| | | 1, 3, 6, 12, 24 h | | | | | | | |
| | | post-inj. | | | | | | | |
| Safety Assessments | | X | X | X | X | X | X | X | X |
| Completion of Core | | | | | X | | | | |
| Study | | | | | | | | | |
| Completion of | | | | | | | | | X |
| Extended Study | | | | | | | | | |

¹Vital signs were to be assessed at each sensory testing
²ECG was to be performed 6 hours after the study drug was given
³Injection site preparation with Betadine was to be done prior to injection of the study drug
⁴Microdialysis probe insertion was to take place before the study drug was injected. In part 2 of the study, 80% of the study drug was injected prior to microdialysis probe insertion.

### Pharmacokinetics/Pharmacodynamics Assessments

Drug Concentration Measurements

Plasma Concentrations

Blood samples for determining plasma concentrations of bupivacaine and dexamethasone were obtained during each of the 2 study periods immediately before dosing (0 hour); at 0.5, 1, 3, 6, 12, 24, 48, 72, and 96 hours post-injection. At each time, -4 mL venous blood was drawn into EDTA-containing tubes.

Tissue Concentrations

Bupivacaine and dexamethasone tissue concentrations were determined by the microdialysis technique. Microdialysis probes were implanted at the site of injection on the medial lower calf (midway between the knee and ankle). Injections were made into an area of subcutaneous tissue approximately 6 cm×6 cm square (as shown in FIG. J-2). The skin at points A and B were first anesthetized with plain 0.5% lidocaine (1 mL total). A 2-inch, 18-gauge intravenous catheter and needle was then passed through the skin at point A, advanced through the subcutaneous tissue for a distance of approximately 5 cm, and then made to exit the skin at point C. The needle was then removed, leaving the intravenous catheter tip protruding 2-3 mm through the skin. A custom loop microdialysis probe (20 mm “window,” MW cutoff 6000 daltons, primed for 20 min with microdialysate infusing at 10 mcL/min.) was inserted through this distal tip to eventually span approximately the distance C-D in the subcutaneous tissue. After allowing the microdialysis probe to equilibrate for 10 minutes (and obtaining a baseline sample over an additional 10 minutes), the first 40% of the total volume of study medication (6 mL or 3 mL depending on treatment) was injected in a fanwise fashion (4 passes) from point B. The second 40% of medication was injected in an identical manner from point A (adjacent to entrance of above catheter). The final 20% (3 mL or 1.5 mL) of study drug was injected through the intravenous catheter as it was withdrawn from point C to point D, then removed entirely. This was to ensure that the microdialysis probe would come to reside completely in the center of this last 20% of injectate. The time required to inject the entire amount of study medication was approximately 5 minutes. Collection of dialysate was continuous for the first 3 hours (10 mcL/min.)—20 minutes sampling period (9 samples, 200 mcL each). After this 3-hour period, the microdialysis probe was disconnected and capped. Each subsequent sample collection was preceded by an uncapping of the probe, reconnection, and a 10-minute flush with dialysate solution (10 mcL/min.). Twenty-minute collections were then to occur at 5 hours 50 minutes, 11 hours 50 minutes, and 23 hours 50 minutes post-injection. The collection was repeated at 48, 72 and 96 hours, such that the midpoint of dialysate collection corresponded to the sensory testing and the blood draw. The pH of all local tissue dialysis samples were measured at the study site laboratory (the site was to retain -50 mcL). The rest of the dialysate was used for determination of drug concentrations.

Pharmacokinetic Metrics

The following pharmacokinetic metrics were derived from the plasma concentration versus time data and the tissue concentration versus time data:

Primary:

AUCt (ng/mL·h): Area under the plasma concentration-time course profile from time=0 to the last quantifiable concentration.

AUC∞ (ng/mL·h): Area under the plasma concentration-time course profile from time=0 to infinity.

Cmax (ng/mL): Maximum observed plasma/tissue concentration taken directly from the concentration-time course profile.

Secondary:

tmax (h): Time from dosing to maximum observed concentration.

t½ (h): Apparent terminal half-life.

MRT (h): Mean residence time, the estimated mean time that any specific molecule is present in the body

Pharmacodynamic Measures

Sensory Testing

Schedule of Assessments: Sensory testing was to be conducted at baseline, and at 30 minutes, 1, 3, 6, 12, 24, 48, 72, and 96 hours post-injection. Sensory testing included:

Pin prick testing: The evaluator assessed the degree of sensory blockade by administering pin-pricks to the injection site. Assessment was made by lightly tapping the skin using the dull end of a dental needle using sufficient pressure to produce a feeling of sharpness (the pressure needed to produce a feeling of sharpness was determined by testing in a non-affected area). The area was pricked with the needle 3 times and the subject was asked how many pin-pricks were felt. If the subject stated that the pin pricks were felt, the subject was asked how many of the 3 pin-pricks were felt as sharp and how many were felt as touch or pressure. The density of block for each tested area was determined and recorded in the source documents and documented on the CRF as follows:

0=Subject did not feel any pin-pricks

1=Subject felt 2 or 3 (out of 3) pin-pricks as touch or pressure

2=Subject felt 2 or 3 (out of 3) pin-pricks as sharp

If only 2 pin-pricks were felt and 1 was felt as touch or pressure and the other was felt as sharp, or if only 1 pin-prick was felt, the level of 1=touch or pressure was assigned.

Thermal Thresholds: Thermal stimulation for determination of Warmth Detection Threshold (WDT) and Heat Pain Detection Threshold (HPDT) was performed with a custom built thermode-thermocouple starting at 30° C. and increasing at a rate of 1.5° C./s to a cutoff limit of 50° C. The subject was instructed to push a button when a sensation of warmth was detected (WDT) and again when the sensation of pain was perceived (HPDT). These values were recorded and the thermode returned to the baseline temperature. If the limit of 50° C. was reached and the subject did not indicate pain, the thermode automatically returned to baseline. Subjects who did not perceive warmth or pain by 50° C. were rated as 51° C. Each threshold was calculated as the median of three determinations performed with intervals of 10 seconds between each determination.

Somesthetic Testing: Somesthetic evaluations were performed by touching the injected area with an alcohol swab. The subject was asked: “Tell me if you feel any change in temperature when I touch this swab to your skin.” (The swab was applied to a different part of the calf to determine baseline perception). The subject was to answer “yes” if a change was perceived, or “no” if no change was perceived. Responses were scored as 0 (Subject did not feel a change in temperature) or 1 (Subject did feel a change in temperature).

Skin Blood Flow

Schedule of Assessments: Skin blood flow was to be measured at baseline and at 30 minutes, 1, 3, 6, 12, 24, 48, 72, and 96 hours post-injection.

Cutaneous blood flow velocity at all injection sites and at a control site 8 cm more proximal on the left calf were tested at baseline using a LASER Doppler flow probe. Flow velocity at the injection site was corrected for changes in flow at the control site using the formula:

Percent change in blood flow velocity=([(Sₓ−Sₒ)/Sₒ]−[(Cₓ−Cₒ)/Cₒ])×100

where Sₓ is the blood flow velocity at the injection site x minutes after the injection, Sₒ is the baseline blood flow velocity at the injection site, Cₓ is the blood flow velocity at the control site x minutes after the injection, and Cₒ is the baseline blood flow velocity at the control site.

Pharmacokinetic/Pharmacodynamic Evaluation

The relationship between plasma concentrations of bupivacaine and sensory testing was examined for the unilateral 15 mL 40K EDLA 2.5% treatment in Part 1 by directly comparing changes in bupivacaine plasma concentrations and sensory test results over time.

The relationship between local tissue concentrations of bupivacaine and sensory testing was examined across all treatments in Part 2 by directly comparing changes in bupivacaine tissue concentrations and sensory test results over time. Part 1 tissue data were not used for this analysis (See Section 9.8.2).

MRI Assessments

Schedule of Assessments: MRI scanning was to be done at baseline (within 7 days prior to study drug injection), at 3 and 96 hours, 2 (±2 days) weeks, 6 (±2 weeks) weeks post-injection. In Part 1, only those subjects randomized to receive 40K EDLA had MRI evaluations.

Standard MRI sequence parameters were used to visualize:

Microspheres: density signal and distribution in the injected area.

Injected fluid/edema: density signal.

### Subject Disposition

Table J-4 summarizes by treatment group the disposition of the 28 subjects randomized to treatment.

TABLE J-4
Patient Disposition: All Randomized Subjects

| | Treatment Groups | | | | | | |
|---|---|---|---|---|---|---|---|
| | | | | | 15 mL | | |
| | | 15 mL | | 7.5 mL | 40K | 15 mL | |
| | | 40K | | 40K | EDLA | AB | |
| | | EDLA | 15 mL | EDLA | 1.25% | 0.25% | |
| | | 2.5% (R) | AB 0.5% | 2.5% (R) | (R) | (R) | |
| | | + | R | + | + | + | |
| | 15 mL | 15 mL | + | 15 mL | 15 mL | 15 mL | |
| | 40K | 40K | 15 mL | 40K | 40K | AB | |
| | EDLA | EDLA | AB 0.5% | EDLA | EDLA | 0.25% | Overall |
| Category | 2.5% (L) | 2.5% (L) | (L) | 2.5% (L) | 2.5% (L) | (L) | Total |
| Randomized | 4 | 4 | 4 | 6 | 6 | 4 | 28 |
| Completed | 4 (100%) | 4 (100%) | 4 (100%) | 6 (100%) | 6 (100%) | 4 (100%) | 28 |
| | | | | | | | (100%) |
| Discontinued (Total) | 0 | 0 | 0 | 0 | 0 | 0 | 0 |

The disposition of subjects is displayed in FIG. J-3.

Data Sets Analyzed

The Intent-to-Treat Population (ITT) included all 28 subjects who were randomized to study medication and received at least one dose of study medication.

The Evaluable for Pharmacokinetics Population, the Evaluable for Efficacy Population, and the Evaluable for Efficacy and Pharmacokinetics Population were all the same and included the ITT Population less those subjects who were completely excluded for the Evaluable for Efficacy Populations due to protocol violations.

The ITT population was used for the Evaluable for Safety Population.

The number of subjects included in each population is presented in Table J-5.

TABLE J-5

| | | 15 mL 40K | | 7.5 mL 40K | 15 mL 40K | |
|---|---|---|---|---|---|---|
| | | EDLA | | EDLA | EDLA | |
| | 15 mL | 2.5% (R) + | 15 mL AB | 2.5% (R) + | 1.25% (R) + | 15 mL AB |
| | 40K | 15 mL 40K | 0.5% (R) + | 15 mL 40K | 15 mL 40K | 0.25% (R) + |
| | EDLA | EDLA | 15 mL AB | EDLA | EDLA | 15 mL AB |
| | 2.5% (L) | 2.5% (L) | 0.5% (L) | 2.5% (L) | 2.5% (L) | 0.25% (L) |
| Populations | Number of Subjects | | | | | |
| ITT | 4 | 4 | 4 | 6 | 6 | 4 |
| PK | 4 | 4 | 4 | 6 | 5 | 4 |
| Efficacy | 4 | 4 | 4 | 6 | 5 | 4 |
| PK/Efficacy | 4 | 4 | 4 | 6 | 5 | 4 |
| Safety | 4 | 4 | 4 | 6 | 6 | 4 |

Table J-6 lists the absolute doses of bupivacaine and dexamethasone for each treatment group based on the concentration and volume of study medication administered.

TABLE J-6
Total Dose of Bupivacaine and Dexamethasone Delivered

| | Total Dose of | Total Dose of |
|---|---|---|
| | Bupivacaine | Dexamethasone |
| Treatment Group | (mg) | (mcg) |
| 15 mL 40K EDLA 2.5% left calf | 270.0 | 150.0 |
| 15 mL 40K EDLA 2.5% right calf + | 540.0 | 300.0 |
| 15 mL 40K EDLA 2.5% left calf | | |
| 15 mL AB 0.5% right calf + | 150.0 | 0 |
| 15 mL AB 0.5% left calf | | |
| 7.5 mL 40K EDLA 2.5% right calf + | 405.0 | 225.0 |
| 15 mL 40K EDLA 2.5% left calf | | |
| 15 mL 40K EDLA 1.25% right calf + | 405.0 | 225.0 |
| 15 mL 40K EDLA 2.5% left calf | | |
| 15 mL AB 0.25% right calf + | 75.0 | 0 |
| 15 mL AB 0.25% left calf | | |

### Plasma Concentration Data: Part 1

Table J-7 presents the mean plasma bupivacaine and dexamethasone concentrations over time for all treatment groups in Part 1.

TABLE J-7
Plasma Bupivacaine/Dexamethasone Concentrations^{a} (ng/mL) Over Time: Part 1
Evaluable for Pharmacokinetics Population

| BUPIVACAINE | | | DEXAMETHASONE | |
|---|---|---|---|---|
| 15 mL 40K EDLA | 15 mL 40K EDLA | 15 mL AB 0.5% | 15 mL 40K EDLA | 15 mL 40K EDLA |
| 2.5% unilateral left | 2.5% bilateral | bilateral | 2.5% unilateral left | 2.5% bilateral |
| (n = 4) | (n = 4) | (n = 4) | (n = 4) | (n = 4) |

Baseline

| | | | | | |
|---|---|---|---|---|---|
| n | 4 | 3 | 2 | 4 | 4 |
| Mean ± SEM | 0 ± 0 | 0 ± 0 | 0 | 0 ± 0 | 0 ± 0 |
| Median | 0 | 0 | 0 | 0 | 0 |
| Min, Max | 0, 0 | 0, 0 | 0, 0 | 0, 0 | 0, 0 |
| 30 min | | | | | |
| n | 4 | 4 | 3 | 4 | 4 |
| Mean ± SEM | 34.3 ± 6.5 | 63.3 ± 5.3 | 447.0 ± 85.8 | 0.01 ± 0.01 | 0.01 ± 0.01 |
| Median | 33.5 | 61.8 | 463.0 | 0 | 0 |
| Min, Max | 20.0, 50.3 | 53.0, 76.7 | 291.0, 587.0 | 0, 0.05 | 0, 0.05 |
| 1 h | | | | | |
| n | 4 | 4 | 4 | 4 | 4 |
| Mean ± SEM | 31.4 ± 8.9 | 67.2 ± 3.4 | 334.0 ± 58.4 | 0 ± 0 | 0.05 ± 0.02 |
| Median | 27.1 | 68.0 | 346.5 | 0 | 0.06 |
| Min, Max | 15.1, 56.1 | 58.3, 74.6 | 209.0, 434.0 | 0, 0 | 0, 0.07 |
| 3 h | | | | | |
| n | 4 | 3 | 4 | 4 | 3 |
| Mean ± SEM | 17.0 ± 4.3 | 29.6 ± 4.4 | 117.7 ± 18.0 | 0.05 ± 0.02 | 0.06 ± 0.03 |
| Median | 16.5 | 27.8 | 109.0 | 0.05 | 0.07 |
| Min, Max | 7.7, 27.2 | 23.1, 38.0 | 84.8, 168.0 | 0, 0.08 | 0, 0.1 |
| 6 h | | | | | |
| n | 4 | 4 | 4 | 4 | 4 |
| Mean ± SEM | 19.0 ± 6.3 | 41.1 ± 4.1 | 89.8 ± 19.9 | 0.09 ± 0.01 | 0.18 ± 0.01 |
| Median | 19.8 | 40.6 | 89.9 | 0.09 | 0.18 |
| Min, Max | 5.5, 31.0 | 31.9, 51.4 | 45.4, 134.0 | 0.06, 0.12 | 0.16, 0.2 |
| 12 h | | | | | |
| n | 4 | 4 | 4 | 4 | 4 |
| Mean ± SEM | 37.2 ± 12.9 | 65.9 ± 9.5 | 75.3 ± 14.4 | 0.17 ± 0.02 | 0.3 ± 0.02 |
| Median | 36.9 | 62.1 | 73.3 | 0.16 | 0.29 |
| Min, Max | 11.7, 63.4 | 49.0, 90.5 | 48.6, 106.0 | 0.13, 0.23 | 0.26, 0.36 |
| 24 h | | | | | |
| n | 4 | 4 | 4 | 4 | 4 |
| Mean ± SEM | 101.8 ± 36.3 | 196.0 ± 30.1 | 81.9 ± 22.9 | 0.28 ± 0.03 | 0.45 ± 0.07 |
| Median | 82.6 | 207.0 | 77.7 | 0.26 | 0.44 |
| Min, Max | 39.2, 203.0 | 119.0, 251.0 | 32.3, 140.0 | 0.23, 0.37 | 0.31, 0.61 |
| 48 h | | | | | |
| n | 4 | 3 | 4 | 4 | 4 |
| Mean ± SEM | 136.1 ± 16.4 | 331.7 ± 58.2 | 49.7 ± 11.3 | 0.08 ± 0.01 | 0.18 ± 0.02 |
| Median | 150.0 | 298.0 | 51.0 | 0.08 | 0.19 |
| Min, Max | 87.3, 157.0 | 252.0, 445.0 | 20.9, 76.1 | 0.06, 0.11 | 0.12, 0.23 |
| 72 h | | | | | |
| n | 4 | 3 | 4 | 4 | 3 |
| Mean ± SEM | 126.0 ± 12.9 | 288.3 ± 82.6 | 20.9 ± 4.3 | 0.01 ± 0.01 | 0.02 ± 0.02 |
| Median | 125.5 | 295.0 | 20.1 | 0 | 0 |
| Min, Max | 96.9, 156.0 | 142.0, 428.0 | 11.8, 31.5 | 0, 0.06 | 0, 0.06 |
| 96 h | | | | | |
| n | 4 | 4 | 4 | 4 | 4 |
| Mean ± SEM | 96.3 ± 36.5 | 165.5 ± 28.6 | 9.1 ± 1.1 | 0 ± 0 | 0 ± 0 |
| Median | 74.5 | 155.5 | 9.0 | 0 | 0 |
| Min, Max | 35.0, 201.0 | 107.0, 244.0 | 6.6, 11.7 | 0, 0 | 0, 0 |

^{a}Note that the maximal concentration and time to maximal concentration reflected in this table may differ from the calculated Cmax and tmax in the pharmacokinetic metrics data.

### Plasma Concentration Data: Part 2

Table J-8 presents the mean plasma bupivacaine and dexamethasone concentrations over time for all treatment groups in Part 2.

TABLE J-8
Plasma Bupivacaine/Dexamethasone Concentrations^{a} (ng/mL) Over Time: Part 2
Evaluable for Pharmacokinetics Population

| BUPIVACAINE | | | DEXAMETHASONE | |
|---|---|---|---|---|
| 7.5 mL 40K EDLA | 15 mL 40K EDLA | | 7.5 mL 40K EDLA | 15 mL 40K EDLA |
| 2.5% (R) + | 1.25% (R) + | | 2.5% (R) + | 1.25% (R) + |
| 15 mL 40K EDLA | 15 mL 40K EDLA | 15 mL AB 0.25% | 15 mL 40K EDLA | 15 mL 40K EDLA |
| 2.5% (L) | 2.5% (L) | bilateral | 2.5% (L) | 2.5% (L) |
| (n = 6) | (n = 5) | (n = 4) | (n = 6) | (n = 5) |

Baseline

| | | | | | |
|---|---|---|---|---|---|
| N | 6 | 5 | 4 | 6 | 5 |
| Mean ± SEM | 0 ± 0 | 0 ± 0 | 0 ± 0 | 0 ± 0 | 0 ± 0 |
| Median | 0 | 0 | 0 | 0 | 0 |
| Min, Max | 0, 0 | 0, 0 | 0, 0 | 0, 0 | 0, 0 |
| 30 min | | | | | |
| n | 6 | 5 | 4 | 6 | 5 |
| Mean ± SEM | 69.5 ± 9.5 | 67.3 ± 12.5 | 201.8 ± 43.8 | 0 ± 0 | 0.02 ± 0.02 |
| Median | 68.9 | 59.0 | 206.0 | 0 | 0 |
| Min, Max | 41.8, 110.0 | 40.0, 99.5 | 114.0, 281.0 | 0, 0 | 0, 0.08 |
| 1 h | | | | | |
| n | 6 | 5 | 4 | 6 | 5 |
| Mean ± SEM | 65.7 ± 10.5 | 55.3 ± 10.5 | 154.3 ± 42.3 | 0 ± 0 | 0.04 ± 0.02 |
| Median | 60.1 | 54.2 | 143.0 | 0 | 0.05 |
| Min, Max | 42.3, 113.0 | 26.7, 78.7 | 70.1, 261.0 | 0, 0 | 0, 0.07 |
| 3 h | | | | | |
| n | 6 | 5 | 4 | 6 | 5 |
| Mean ± SEM | 44.3 ± 9.0 | 35.8 ± 9.3 | 72.1 ± 22.6 | 0.02 ± 0.01 | 0.06 ± 0.02 |
| Median | 36.6 | 32.2 | 64.6 | 0 | 0.06 |
| Min, Max | 24.8, 78.8 | 15.0, 61.1 | 29.1, 130.0 | 0, 0.07 | 0, 0.1 |
| 6 h | | | | | |
| n | 6 | 5 | 4 | 6 | 5 |
| Mean ± SEM | 49.4 ± 11.5 | 42.2 ± 11.2 | 54.9 ± 14.5 | 0.14 ± 0.01 | 0.16 ± 0.01 |
| Median | 45.4 | 31.3 | 48.5 | 0.15 | 0.15 |
| Min, Max | 24.2, 101.0 | 18.5, 75.9 | 30.0, 92.6 | 0.07, 0.16 | 0.12, 0.19 |
| 12 h | | | | | |
| n | 6 | 5 | 4 | 6 | 5 |
| Mean ± SEM | 84.3 ± 22.8 | 63.8 ± 11.5 | 39.1 ± 9.1 | 0.24 ± 0.03 | 0.27 ± 0.03 |
| Median | 63.7 | 66.7 | 39.2 | 0.26 | 0.3 |
| Min, Max | 38.2, 193.0 | 35.3, 97.0 | 21.1, 56.7 | 0.14, 0.31 | 0.19, 0.34 |
| 24 h | | | | | |
| n | 6 | 5 | 4 | 6 | 5 |
| Mean ± SEM | 152.5 ± 33.2 | 135.7 ± 19.3 | 34.0 ± 8.1 | 0.23 ± 0.02 | 0.33 ± 0.04 |
| Median | 124.0 | 147.0 | 34.5 | 0.23 | 0.37 |
| Min, Max | 82.9, 295.0 | 87.7, 190 | 13.9, 53.0 | 0.15, 0.3 | 0.21, 0.42 |
| 48 h | | | | | |
| n | 6 | 5 | 4 | 6 | 5 |
| Mean ± SEM | 133.5 ± 25.1 | 158.4 ± 19.6 | 17.1 ± 6.4 | 0.08 ± 0.01 | 0.1 ± 0.03 |
| Median | 111.4 | 165.0 | 14.2 | 0.07 | 0.09 |
| Min, Max | 78.0, 228.0 | 105.0, 218.0 | 5.6, 34.5 | 0.05, 0.1 | 0, 0.17 |
| 72 h | | | | | |
| n | 6 | 5 | 4 | 6 | 5 |
| Mean ± SEM | 58.2 ± 10.1 | 95.8 ± 21.2 | 7.3 ± 4.5 | 0 ± 0 | 0 ± 0 |
| Median | 59.5 | 92.7 | 5.1 | 0 | 0 |
| Min, Max | 29.7, 94.2 | 46.0, 173.0 | 0, 18.8 | 0, 0 | 0, 0 |
| 96 h | | | | | |
| n | 6 | 5 | 4 | 6 | 5 |
| Mean ± SEM | 30.0 ± 4.1 | 48.1 ± 9.7 | 1.5 ± 1.5 | 0 ± 0 | 0 ± 0 |
| Median | 28.7 | 46.5 | 0 | 0 | 0 |
| Min, Max | 19.2, 48.5 | 18.5, 71.8 | 0, 5.8 | 0, 0 | 0, 0 |

R = right side;
L = left side
^{a}Note that the maximal concentration and time to maximal concentration reflected in this table may differ from the calculated Cmax and tmax in the pharmacokinetic metrics data.

### Plasma Pharmacokinetic Metrics: Parts 1 and 2

Table J-9(A) presents the pharmacokinetic metrics for the plasma concentrations of bupivacaine and dexamethasone in Part 1. Table J-9(B) presents the pharmacokinetic metrics for the plasma concentrations of bupivacaine and dexamethasone in Part 2.

TABLE J-9(A)
Plasma Bupivacaine/Dexamethasone Pharmacokinetic Metrics: Part 1
Evaluable for Pharmacokinetics Population

| Bupivacaine | | | Dexamethasone | |
|---|---|---|---|---|
| 15 mL 40K EDLA | 15 mL 40K EDLA | | 15 mL 40K EDLA | |
| 2.5% | 2.5% | 15 mL AB 0.5% | 2.5% | 15 mL 40K EDLA |
| unilateral left | bilateral | bilateral | unilateral left | 2.5% bilateral |
| (n = 4) | (n = 4) | (n = 4) | (n = 4) | (n = 4) |

Cmax (ng/mL)

| | | | | | |
|---|---|---|---|---|---|
| n | 4 | 4 | 4 | 4 | 4 |
| Mean ± SEM | 162.0 ± 25.3 | 287.8 ± 60.2 | 443.5 ± 60.8 | 0.28 ± 0.03 | 0.45 ± 0.07 |
| Median | 174.0 | 275.0 | 448.0 | 0.26 | 0.44 |
| Min, Max | 96.9, 203.0 | 156.0, 445.0 | 291.0, 587.0 | 0.23, 0.37 | 0.31, 0.61 |
| AUCt (ng/mL · h) | | | | | |
| n | 4 | 4 | 4 | 4 | 4 |
| Mean ± SEM | 9744.2 ± 1473.4 | 19220.4 ± 3833.6 | 5218.2 ± 1003.7 | 8.5 ± 1.5 | 14.4 ± 1.9 |
| Median | 10350.3 | 17999.9 | 5093.5 | 7.2 | 14.5 |
| Min, Max | 5710.1, 12566.1 | 11403.3, 29478.4 | 2903.2, 7782.5 | 6.5, 12.9 | 10.2, 18.5 |
| AUC∞(ng/mL · h) | | | | | |
| n | 0 | 0 | 4 | 0 | 0 |
| Mean ± SEM | | | 5497.9 ± 1015.8 | | |
| Median | | | 5349.8 | | |
| Min, Max | | | 3178.4, 8113.5 | | |
| tmax (h) | | | | | |
| n | 4 | 4 | 4 | 4 | 4 |
| Mean ± SEM | 59.4 ± 14.8 | 58.9 ± 12.0 | 0.81 ± 0.14 | 22.5 ± 0.6 | 19.9 ± 3.1 |
| Median | 60.3 | 47.5 | 0.79 | 22.6 | 22.3 |
| Min, Max | 23.8, 93.4 | 45.5, 94.9 | 0.5, 1.2 | 21.0, 23.7 | 10.7, 24.4 |
| t1/2 (h) | | | | | |
| n | 2 | 1 | 4 | 0 | 0 |
| Mean ± SEM | 48.5 ± 14.1 | 37.7 | 22.1 ± 2.3 | | |
| Median | 48.5 | 37.7 | 19.9 | | |
| Min, Max | 34.4, 62.7 | 37.7, 37.7 | 19.4, 29.0 | | |
| MRT (h) | | | | | |
| n | 4 | 4 | 4 | 4 | 4 |
| Mean ± SEM | 53.6 ± 2.9 | 53.2 ± 2.2 | 28.1 ± 1.6 | 23.7 ± 1.3 | 24.4 ± 1.2 |
| Median | 52.5 | 54.2 | 27.8 | 23.3 | 23.2 |
| Min, Max | 48.5, 60.9 | 47.2, 57.4 | 24.6, 32.2 | 21.1, 27.2 | 23.1, 28.1 |

In Part 1 of the study (Table J-9(A)), the bilateral 15 mL AB 0.5% treatment demonstrated rapid distribution of bupivacaine into the systemic circulation, characterized by a relatively low mean tmax (0.81 hours). In contrast, both the unilateral and bilateral 15 mL 40K EDLA 2.5% treatments demonstrated a slow distribution of bupivacaine into the systemic circulation (mean tmax=59.4 and 58.9 hours, respectively). The mean tmax for dexamethasone was lower than that of bupivacaine and was similar across the unilateral and bilateral 40K EDLA treatment groups (22.5 hours for unilateral, 19.9 hours for bilateral). The mean t1/2 and MRT were longer for both the unilateral and bilateral 40K EDLA treatments than the mean t1/2 and MRT for the AB treatment.

In order to determine the dose-proportionality of bupivacaine concentrations in plasma after treatment with increasing 40K EDLA doses, the Cmax and AUCt for the unilateral and bilateral 15 mL 40K EDLA 2.5% treatments were compared. The mean plasma Cmax for the bilateral 15 mL 40K EDLA 2.5% treatment (287.8 ng/mL) was slightly less than double the Cmax of the unilateral treatment (162.0 ng/mL). Similarly, the mean AUCt achieved with the bilateral 15 mL 40K EDLA 2.5% treatment (19220.4 ng/mL·h) was slightly less than double that of the unilateral treatment (9744.2 ng/mL·h). Dexamethasone concentrations showed a similar dose-proportional relationship. The Cmax resulting from the bilateral 15 mL 40K EDLA 2.5% treatment (0.45 ng/mL) was slightly less than double that of the unilateral treatment (0.28 ng/mL). Similarly, the mean AUCt achieved with the bilateral 15 mL 40K EDLA 2.5% treatment (14.4 ng/mL·h) was slightly less than double that of the unilateral treatment (8.5 ng/mL·h).

The Cmax of both the unilateral and bilateral 15 mL 40K EDLA 2.5% treatments was less than that of the bilateral AB treatment (Cmax=443.5 ng/mL), despite a greater total bupivacaine dose in the 40K EDLA-treated subjects (unilateral 40K EDLA=270.0 mg bupivacaine, bilateral 40K EDLA=540.0 mg bupivacaine, bilateral AB=150.0 mg bupivacaine).

TABLE J-9(B)
Plasma Bupivacaine/Dexamethasone Pharmacokinetic Metrics: Part 2
Evaluable for Pharmacokinetics Population

| | Bupivacaine | | | Dexamethasone | |
|---|---|---|---|---|---|
| | 7.5 mL 40K EDLA | 15 mL 40K EDLA | | 7.5 mL 40K EDLA | 15 mL 40K EDLA |
| | 2.5% (R) + | 1.25% (R) + | | 2.5% (R) + | 1.25% (R) + |
| | 15 mL 40K EDLA | 15 mL 40K EDLA | 15 mL AB 0.25% | 15 mL 40K EDLA | 15 mL 40K EDLA |
| | 2.5% (L) | 2.5% (L) | bilateral | 2.5% (L) | 2.5% (L) |
| | (n = 6) | (n = 5) | (n = 4) | (n = 6) | (n = 5) |
| Cmax (ng/mL) | | | | | |
| n | 6 | 5 | 4 | 6 | 5 |
| Mean ± SEM | 153.1 ± 33.1 | 170.8 ± 18.7 | 201.8 ± 43.8 | 0.25 ± 0.02 | 0.33 ± 0.04 |
| Median | 125.0 | 176.0 | 206.0 | 0.27 | 0.37 |
| Min, Max | 82.9, 295.0 | 105.0, 218.0 | 114.0, 281.0 | 0.17, 0.31 | 0.21, 0.42 |
| AUCt | | | | | |
| (ng/mL · h) | | | | | |
| n | 6 | 5 | 4 | 6 | 5 |
| Mean ± SEM | 8924.9 ± 1719.8 | 9935.2 ± 979.8 | 2141.7 ± 493.7 | 7.6 ± 0.69 | 9.7 ± 1.7 |
| Median | 7282.9 | 10652.6 | 2173.2 | 8.0 | 11.7 |
| Min, Max | 5065.5, 15890.3 | 6529.3, 12185.9 | 904.7, 3315.8 | 5.4, 10.0 | 3.9, 13.1 |
| AUC∞ | | | | | |
| (ng/mL · h) | | | | | |
| n | 6 | 2 | 1 | 0 | 0 |
| Mean ± SEM | 10082.6 ± 1793.5 | 10768.4 ± 1140.3 | 2311.8 | | |
| Median | 8286.3 | 10768.4 | 2311.8 | | |
| Min, Max | 5722.3, 16547.2 | 9628.1, 11908.8 | 2311.8, 2311.8 | | |
| tmax (h) | | | | | |
| n | 6 | 5 | 4 | 6 | 5 |
| Mean ± SEM | 31.3 ± 4.9 | 42.3 ± 5.0 | 0.71 ± 0.03 | 16.0 ± 2.6 | 20.9 ± 2.2 |
| Median | 24.4 | 46.9 | 0.73 | 12.6 | 23.0 |
| Min, Max | 22.5, 46.9 | 22.6, 48.7 | 0.62, 0.77 | 10.6, 25.6 | 12.0, 23.3 |
| t½ (h) | | | | | |
| n | 6 | 4 | 1 | 0 | 0 |
| Mean ± SEM | 25.8 ± 2.6 | 32.9 ± 7.9 | 26.5 | | |
| Median | 25.9 | 31.4 | 26.5 | | |
| Min, Max | 16.0, 34.5 | 17.1, 51.9 | 26.5, 26.5 | | |
| MRT (h) | | | | | |
| n | 6 | 5 | 4 | 6 | 5 |
| Mean ± SEM | 41.2 ± 1.5 | 45.1 ± 2.6 | 20.5 ± 4.3 | 21.7 ± 0.5 | 20.7 ± 1.9 |
| Median | 41.2 | 45.6 | 20.6 | 21.5 | 22.5 |
| Min, Max | 37.0, 45.6 | 36.2, 52.8 | 10.9, 29.8 | 20.5, 23.4 | 13.1, 23.2 |

In Part 2 of the study, one 40K EDLA-treated group received 7.5 mL 40K EDLA 2.5%+15 mL 40K EDLA 2.5%, and the other received 15 mL 40K EDLA 1.25%+15 mL 40K EDLA 2.5%. Although both treatments delivered the same total dose of bupivacaine, the mean Cmax, AUCt, and tmax were slightly higher for the 15 mL 40K EDLA 1.25%+15 mL 40K EDLA 2.5% treatment. However, the variability for these parameters is somewhat large. Although the absolute dose of dexamethasone was also the same with these two treatments, the mean Cmax, AUCt, and tmax were also slightly higher for the 15 mL 40K EDLA 1.25%+15 mL 40K EDLA 2.5% treatment. As in Part 1, the mean Cmax for either 40K EDLA treatment (153.1 and 170.8 ng/mL) was less than the Cmax for AB (201.8 ng/mL), despite a larger total bupivacaine dose for 40K EDLA treatment (405.0 mg bupivacaine) than for AB treatment (75.0 mg bupivacaine).

As in Part 1 of the study, treatment with AB in Part 2 resulted in a rapid distribution of bupivacaine into plasma (tmax=0.71 hours) compared to 7.5 mL 40K EDLA 2.5%+15 mL 40K EDLA 2.5% (tmax=31.3 hours) or 15 mL 40K EDLA 1.25%+15 mL 40K EDLA 2.5% (tmax=42.3 hours). In addition, the mean MRT for 40K EDLA treatment was approximately double that for AB treatment (41.2 and 45.1 hours for 40K EDLA vs. 20.5 hours for AB). The mean MRT for dexamethasone (21.7 and 20.7 hours) was approximately half that of bupivacaine (41.2 and 45.1 hours) with 40K EDLA treatment.

### Tissue Bupivacaine and Dexamethasone

Tissue Concentration Data: Part 1

In Part 1 of the study, the tissue concentrations of bupivacaine were often too large to be measured under the assay conditions employed; therefore there are no summary statistics for bupivacaine tissue concentrations or pharmacokinetic metrics for Part 1.

Tissue Concentration Data: Part 2

The tissue concentrations of bupivacaine and dexamethasone at selected time points for Part 2 are presented in Tables J-10(A) (bupivacaine) and J-10(B) (dexamethasone).

TABLE J-10 (A)
Tissue Bupivacaine Concentrations^{a} (ng/mL) Over Time: Part 2
Evaluable for Pharmacokinetics Population

| | | | | | |
|---|---|---|---|---|---|
| 7.5 mL 40K | 15 mL 40K EDLA | 15 mL 40K EDLA | 15 mL 40K EDLA | 15 mL AB | 15 mL AB |
| EDLA 2.5% right | 2.5% left | 1.25% rignt | 2.5% left | 0.25% right | 0.25% left |

1 h

| | | | | | | |
|---|---|---|---|---|---|---|
| N | 6 | 5 | 4 | 5 | 4 | 4 |
| Mean | 17414.5 | 17644.6 | 16660.0 | 16124.8 | 5413.3 | 7139.5 |
| ±SEM | 2605.0 | 4778.6 | 1236.6 | 5030.9 | 1860.6 | 2754.5 |
| Median | 15919.5 | 18799.0 | 17090.5 | 14434.0 | 6016.0 | 6878.0 |
| Min, Max | 11208.0, 26332.0 | 5057.0, 29680.0 | 13387.0, 19072.0 | 5547.0, 32380.0 | 801.0, 8820.0 | 1207.0, 13595.0 |
| 2 h | | | | | | |
| n | 6 | 6 | 5 | 5 | 4 | 4 |
| Mean | 24758.5 | 21052.5 | 15994.6 | 16242 | 4998.8 | 5038.5 |
| ±SEM | 3581.2 | 4388.5 | 3316.9 | 5243.4 | 1018.8 | 788.7 |
| Median | 24412.5 | 17951.5 | 17562.0 | 13578.0 | 4649.5 | 5023.5 |
| Min, Max | 16389.0, 34102.0 | 9318.0, 38447.0 | 3508.0, 22643.0 | 5691.0, 35068.0 | 3265.0, 7431.0 | 3566.0, 6541.0 |
| 3 h | | | | | | |
| n | 6 | 6 | 5 | 5 | 4 | 4 |
| Mean | 19329.3 | 26736.8 | 16711.6 | 22440.0 | 4040.5 | 4639.8 |
| ±SEM | 2731.2 | 4214.8 | 3584.9 | 6335.4 | 557.1 | 678.6 |
| Median | 16617.0 | 28227.0 | 19886.0 | 20540.0 | 3914.0 | 4576.0 |
| Min, Max | 14002.0, 31201.0 | 10672.0, 39925.0 | 4038.0, 25045.0 | 8060.0, 45305.0 | 3068.0, 5266.0 | 3394.0, 6013.0 |
| 5 h 50 min | | | | | | |
| n | 6 | 5 | 5 | 5 | 4 | 4 |
| Mean | 31163.8 | 37953.4 | 31936.6 | 32427.8 | 4234.3 | 6480.8 |
| ±SEM | 3233.3 | 4165.3 | 7184.8 | 9544.4 | 879.9 | 824.5 |
| Median | 30529.0 | 33703.0 | 36366.0 | 23865.0 | 4262.5 | 6034.0 |
| Min, Max | 20788.0, 40191.0 | 30559.0, 53932.0 | 12099.0, 46827.0 | 15502.0, 68927.0 | 2271.0, 6141.0 | 5157.0, 8698.0 |
| 11 h 50 min | | | | | | |
| n | 6 | 6 | 5 | 5 | 4 | 4 |
| Mean | 34417.2 | 45942.2 | 33970.2 | 42937.4 | 4083.8 | 4320.5 |
| ±SEM | 4617.8 | 6168.0 | 7349.3 | 9677.5 | 915.0 | 1498.0 |
| Median | 35965.5 | 41928.0 | 23606.0 | 30695.0 | 4501.0 | 3114.0 |
| Min, Max | 15927.0, 46082.0 | 28378.0, 73464.0 | 20913.0, 56611.0 | 26925.0, 77624.0 | 1529.0, 5804.0 | 2335.0, 8719.0 |
| 23 h 50 min | | | | | | |
| n | 5 | 5 | 5 | 5 | 4 | 4 |
| Mean | 32596.6 | 58099.8 | 35307.4 | 54570.8 | 678.5 | 1972.0 |
| ±SEM | 1922.7 | 4626.2 | 7114.1 | 10250.8 | 205.3 | 851.1 |
| Median | 31643.0 | 54984.0 | 29561.0 | 41365.0 | 678.5 | 1895.5 |
| Min, Max | 28026.0, 38213.0 | 48036.0, 74031.0 | 17891.0, 59562.0 | 32246.0, 82523.0 | 209.0, 1148.0 | 400.0, 3697.0 |
| 48 h | | | | | | |
| n | 6 | 6 | 5 | 4 | 3 | 3 |
| Mean | 27433.0 | 48370.3 | 26152.4 | 39954.8 | 154.8 | 296.0 |
| ±SEM | 5288.2 | 4414.7 | 7448.5 | 9671.5 | 13.0 | 96.7 |
| Median | 27032.0 | 50279.0 | 17948.0 | 33238.5 | 164.4 | 382.0 |
| Min, Max | 10477.0, 42081.0 | 34706.0, 61665.0 | 10243.0, 49720.0 | 25024.0, 68318.0 | 129.0, 171.0 | 103.0, 403.0 |
| 72 h | | | | | | |
| n | 6 | 6 | 4 | 5 | 4 | 3 |
| Mean | 31131.5 | 31411.5 | 15272.3 | 34906.4 | 154.8 | 175.6 |
| ±SEM | 10755.0 | 7229.4 | 4026.4 | 7811.3 | 79.1 | 79.0 |
| Median | 22989.5 | 30676.0 | 15228.0 | 35286.0 | 85.8 | 108.0 |
| Min, Max | 7536.0, 79949.0 | 3361.0, 58470.0 | 6725.0, 23908.0 | 10429.0, 57957.0 | 56.6, 391.0 | 85.7, 333.0 |
| 96 h | | | | | | |
| N | 6 | 6 | 5 | 5 | 4 | 3 |
| Mean | 12248.3 | 15842.3 | 19816.0 | 12771.2 | 125.0 | 105.0 |
| ±SEM | 4341.9 | 4242.0 | 7376.1 | 3266.0 | 69.6 | 66.1 |
| Median | 12724.5 | 16562.5 | 14283.0 | 12811.0 | 88.1 | 88.1 |
| Min, Max | 834.0, 27913.0 | 948.0, 32110.0 | 5668.0, 44279.0 | 3964.0, 23121.0 | 0.0, 324.0 | 0.0, 227.0 |

^{a}Note that the maximal concentration and time to maximal concentration reflected in this table may differ from the calculated Cmax and tmax in the pharmacokinetic metrics data.

TABLE J-10(B)
Tissue Dexamethasone Concentrations^{a} (ng/mL) Over Time: Part 2
Evaluable for Pharmacokinetics Population

| | | | 15 mL 40K | |
|---|---|---|---|---|
| | 7.5 mL 40K | 15 mL 40K | EDLA 1.25% | 15 mL 40K |
| | EDLA 2.5% right | EDLA 2.5% left | right | EDLA 2.5% left |
| 1 h | | | | |
| n | 6 | 5 | 4 | 5 |
| Mean ± SEM | 22.4 ± 6.4 | 30.8 ± 10.9 | 23.0 ± 5.7 | 20.1 ± 5.5 |
| Median | 21.8 | 26.4 | 25.7 | 15.1 |
| Min, Max | 3.5, 42.0 | 5.1, 66.3 | 6.8, 33.7 | 10.1, 41.3 |
| 2 h | | | | |
| n | 6 | 6 | 5 | 5 |
| Mean ± SEM | 29.2 ± 7.5 | 25.3 ± 6.4 | 16.2 ± 4.6 | 18.8 ± 3.5 |
| Median | 25.4 | 21.4 | 19.4 | 20.5 |
| Min, Max | 6.3, 52.6 | 10.3, 50.4 | 3.2, 27.3 | 8.3, 28.1 |
| 3 h | | | | |
| n | 6 | 6 | 5 | 5 |
| Mean ± SEM | 21.5 ± 6.6 | 27.5 ± 5.4 | 19.5 ± 4.3 | 16.1 ± 3.6 |
| Median | 20.4 | 27.3 | 22.6 | 14.0 |
| Min, Max | 5.4, 51.0 | 9.0, 46.3 | 3.6, 27.5 | 7.6, 24.8 |
| 5 h 50 min | | | | |
| n | 6 | 5 | 5 | 5 |
| Mean ± SEM | 41.8 ± 9.5 | 82.6 ± 23.8 | 61.9 ± 17.0 | 62.1 ± 24.6 |
| Median | 35.8 | 65.8 | 66.6 | 37.2 |
| Min, Max | 21.0, 75.0 | 34.9, 156.0 | 7.0, 111.0 | 14.5, 126.0 |
| 11 h 50 min | | | | |
| n | 6 | 6 | 5 | 5 |
| Mean ± SEM | 47.3 ± 7.9 | 66.7 ± 19.1 | 36.6 ± 14.5 | 84.0 ± 34.3 |
| Median | 40.8 | 48.2 | 27.4 | 65.4 |
| Min, Max | 28.2, 77.3 | 20.0, 142.0 | 8.7, 92.1 | 22.1, 212.0 |
| 23 h 50 min | | | | |
| n | 5 | 5 | 5 | 5 |
| Mean ± SEM | 18.8 ± 5.3 | 35.4 ± 10.0 | 21.4 ± 11.3 | 50.8 ± 24.1 |
| Median | 13.2 | 27.5 | 11.9 | 31.5 |
| Min, Max | 11.8, 39.5 | 20.7, 74.9 | 4.6, 66.0 | 17.3, 146.0 |
| 48 h | | | | |
| n | 6 | 6 | 5 | 4 |
| Mean ± SEM | 10.8 ± 4.0 | 27.5 ± 11.6 | 9.0 ± 5.7 | 20.8 ± 6.6 |
| Median | 71 | 12.5 | 2.5 | 187 |
| Min, Max | 2.2, 29.4 | 7.9, 78.1 | 1.4, 31.1 | 7.0, 38.7 |
| 72 h | | | | |
| n | 6 | 6 | 4 | 5 |
| Mean ± SEM | 11.0 ± 6.2 | 9.1 ± 4.3 | 3.6 ± 2.5 | 10.1 ± 3.8 |
| Median | 5.5 | 6.3 | 1.7 | 6.2 |
| Min, Max | 1.5, 41.4 | 0.7, 30.2 | 0.0, 10.8 | 2.7, 20.8 |
| 96 h | | | | |
| n | 6 | 6 | 5 | 5 |
| Mean ± SEM | 16.1 ± 11.8 | 4.1 ± 1.7 | 7.9 ± 3.6 | 2.9 ± 1.1 |
| Median | 2.6 | 3.0 | 6.4 | 2.8 |
| Min, Max | 0.0, 73.8 | 0.7, 12.5 | 0.0, 18.5 | 0.0, 6.7 |

^{a}Note that the maximal concentration and time to maximal concentration reflected in this table may differ from the calculated Cmax and tmax in the pharmacokinetic metrics data.

Tissue Pharmacokinetic Metrics: Part 2

Tables J-11(A) and J-11(B) present the pharmacokinetic metrics for the tissue concentrations of bupivacaine and dexamethasone, respectively, for Part 2 of the study.

TABLE J-11 (A)
Tissue Bupivacaine Pharmacokinetic Metrics: Part 2
Evaluable for Pharmacokinetics Population

| 7.5 mL 40K | 15 mL 40K | 15 mL 40K EDLA | 15 mL 40K | 15 mL AB | |
|---|---|---|---|---|---|
| EDLA 2.5% | EDLA 2.5% | 1.25% | EDLA 2.5% | 0.25% | 15 mL AB 0.25% |
| right | left | right | left | right | left |
| Cmax | | | | | |

(ng/mL)

| | | | | | | |
|---|---|---|---|---|---|---|
| n | 6 | 6 | 5 | 5 | 4 | 4 |
| Mean | 46710.3 | 58472.7 | 41082.2 | 54719.2 | 28919.8 | 24810.8 |
| ±SEM | 7086.9 | 3641.0 | 6461.3 | 10204.0 | 18864.3 | 12079.9 |
| Median | 43338.0 | 56931.0 | 46292.0 | 42107.0 | 13839.5 | 18474.0 |
| Min, Max | 29342.0, 79949.0 | 49495.0, | 23606.0, 59562.0 | 32246.0, | 3265.0, 84735.0 | 5157.0, 57138.0 |
| | | 74031.0 | | 82523.0 | | |
| AUCt | | | | | | |
| (ng/mL·h) | | | | | | |
| n | 6 | 6 | 5 | 5 | 4 | 4 |
| Mean | 2688330.3 | 3756938.8 | 2294113.0 | 3526532.3 | 100563.5 | 137506.0 |
| ±SEM | 296398.2 | 157067.0 | 511460.8 | 585452.2 | 20684.1 | 30401.9 |
| Median | 2726977.8 | 3796442.9 | 1846200.4 | 3244569.7 | 103039.8 | 118976.5 |
| Min, Max | 1625087.4, | 3230823.1, | 1051878.2, | 1877679.5, | 49677.3, | 88472.7, |
| | 3751288.0 | 4332246.3 | 3911488.8 | 5416888.0 | 146497.2 | 223598.4 |
| AUC∞ | | | | | | |
| (ng/mL · h) | | | | | | |
| n | 4 | 4 | 1 | 2 | 3 | 2 |
| Mean | 3774531.1 | 4235629.6 | 1980785.2 | 3371669.1 | 103355.1 | 122687.1 |
| ±SEM | 1168311.0 | 202080.1 | | 1392012.6 | 32750.1 | 32289.4 |
| Median | 3212170.6 | 4227838.6 | 1980785.2 | 3371669.1 | 93930.0 | 122687.1 |
| Min, Max | 1656490.1, | 3866280.4, | 1980785.2, | 1979656.4, | 51933.2, | 90397.7, |
| | 7017293.3 | 4620560.8 | 1980785.2 | 4763681.7 | 164202.1 | 154976.5 |
| tmax (h) | | | | | | |
| n | 6 | 6 | 5 | 5 | 4 | 4 |
| Mean | 31.6 | 33.7 | 15.0 | 32.8 | 0.5 | 1.9 |
| ±SEM | 9.5 | 8.7 | 3.3 | 9.5 | 0.4 | 1.6 |
| Median | 24.1 | 24.4 | 12.0 | 23.1 | 0.05 | 0.31 |
| Min, Max | 11.5, 70.9 | 12.9, 70.9 | 6.2, 22.8 | 22.9, 70.7 | 0.03, 1.8 | 0.27, 6.6 |
| t1/2 (h) | | | | | | |
| n | 5 | 5 | 3 | 2 | 3 | 2 |
| Mean | 46.3 | 32.6 | 49.1 | 25.0 | 34.0 | 36.4 |
| ±SEM | 12.3 | 7.8 | 12.4 | 7.1 | 9.5 | 21.2 |
| Median | 37.3 | 32.9 | 39.5 | 25.0 | 37.9 | 36.4 |
| Min, Max | 15.2, 81.1 | 8.0, 56.0 | 34.1, 73.8 | 17.8, 32.1 | 15.9, 48.3 | 15.2, 57.6 |
| MRT (h) | | | | | | |
| n | 6 | 6 | 5 | 5 | 4 | 4 |
| Mean | 42.4 | 41.7 | 39.1 | 41.0 | 14.6 | 14.2 |
| ±SEM | 3.0 | 2.8 | 1.4 | 2.5 | 2.1 | 2.9 |
| Median | 42.7 | 42.0 | 40.2 | 38.1 | 15.1 | 13.4 |
| Min, Max | 33.7, 54.2 | 30.0, 51.1 | 34.5, 41.9 | 35.7, 47.8 | 9.6, 18.6 | 8.0, 21.8 |

TABLE J-11(B)
Tissue Dexamethasone Pharmacokinetic Metrics: Part 2
Evaluable for Pharmacokinetics Population

| | 7.5 mL 40K EDLA | | | |
|---|---|---|---|---|
| | 2.5% | 15 mL 40K EDLA 2.5% | 15 mL 40K EDLA 1.25% | 15 mL 40K EDLA 2.5% |
| | right | left | right | left |
| Cmax (ng/mL) | | | | |
| n | 6 | 6 | 5 | 5 |
| Mean ± SEM | 58.4 ± 7.9 | 91.5 ± 20.7 | 62.2 ± 16.7 | 97.3 ± 32.9 |
| Median | 61.2 | 85.4 | 66.6 | 90.3 |
| Min, Max | 32.5, 77.3 | 37.4, 156.0 | 8.7, 111.0 | 30.2, 212.0 |
| AUCt (ng/mL · h) | | | | |
| n | 6 | 6 | 5 | 5 |
| Mean ± SEM | 1831.2 ± 381.2 | 2495.8 ± 549.8 | 1396.5 ± 588.6 | 2723.6 ± 989.6 |
| Median | 1652.0 | 1889.3 | 851.7 | 1976.2 |
| Min, Max | 860.6, 3172.7 | 1317.9, 4297.6 | 330.0, 3662.1 | 953.6, 6478.9 |
| AUC∞ | | | | |
| (ng/mL · h) | | | | |
| n | 3 | 6 | 1 | 5 |
| Mean ± SEM | 1451.8 ± 412.2 | 2624.2 ± 560.8 | 4197.8 | 2831.6 ± 984.5 |
| Median | 1211.4 | 1977.3 | 4197.8 | 2047.7 |
| Min, Max | 889.1, 2255.0 | 1464.8, 4444.9 | 4197.8, 4197.8 | 1021.6, 6510.9 |
| tmax(h) | | | | |
| N | 6 | 6 | 5 | 5 |
| Mean ± SEM | 24.0 ± 14.3 | 9.1 ± 1.2 | 7.4 ± 1.0 | 9.5 ± 1.3 |
| Median | 11.4 | 8.7 | 6.5 | 11.2 |
| Min, Max | 6.3, 95.2 | 6.4, 12.9 | 6.1, 11.5 | 6.2, 12.0 |
| t½ (h) | | | | |
| N | 3 | 6 | 1 | 5 |
| Mean ± SEM | 20.8 ± 2.4 | 21.4 ± 3.2 | 26.9 | 19.0 ± 3.1 |
| Median | 19.0 | 21.2 | 26.9 | 17.7 |
| Min, Max | 17.8, 25.6 | 10.3, 33.2 | 26.9, 26.9 | 11.8, 30.6 |
| MRT (h) | | | | |
| N | 6 | 6 | 5 | 5 |
| Mean ± SEM | 31.7 ± 7.1 | 28.3 ± 2.8 | 25.2 ± 5.1 | 26.9 ± 2.6 |
| Median | 26.6 | 26.9 | 25.8 | 24.8 |
| Min, Max | 18.7, 65.9 | 21.4, 40.6 | 11.1, 40.7 | 21.5, 33.4 |

The reliability of the microdialysis methodology in measuring local tissue concentrations of bupivacaine was assessed by determining the similarity of the tissue pharmacokinetic metrics of bupivacaine obtained at different sites using identical doses of 40K EDLA. For the two 15 mL 40K EDLA 2.5% treatments, the mean tissue Cmax (58472.7 vs. 54719.2 ng/mL) and AUCt (3756938.8 vs. 3526532.3 ng/mL·h) obtained were similar, indicating that the microdialysis method can reliably detect similar peak and total tissue exposure to bupivacaine following a similar dose of bupivacaine delivered as 40K EDLA at two different sites. For the two sites treated with 15 mL AB 0.25%, the tissue Cmax (28919.8 vs. 24810.8 ng/mL) and AUCt (100563.5 vs. 137506.0 ng/mL·h) obtained are also similar, indicating that the method is reliable for detecting tissue bupivacaine delivered as AB as well.

The relationship between 40K EDLA dose and tissue bupivacaine concentration was examined by comparing the tissue pharmacokinetic metrics for bupivacaine obtained at different sites where differing doses of 40K EDLA were delivered (Table 11.2.1.2.3A).

Although the 15 mL EDLA 2.5% treatment delivered twice the dose of bupivacaine as the 7.5 mL 40K EDLA 2.5% treatment, the mean Cmax measured by the assay for the 15 mL 40K EDLA 2.5% treatment was not double that of the 7.5 mL 40K EDLA 2.5% treatment (46710.3 vs. 58472.7 ng/mL, respectively) in the same subjects. Likewise, the mean AUCt for the two treatments (2688330.3 vs. 3756938.8 ng/mL·h, respectively) in the same subjects did not reflect a doubling of total exposure between the two treatments. Since the injection volume differed between the two treatments, it is possible that this difference may have affected the total dose of bupivacaine delivered at the tissue site.

However, a comparison of two treatments in the same subjects in which one delivers twice the dose of bupivacaine but utilizes a similar injection volume (15 mL 40K EDLA 1.25% vs. 15 mL 40K EDLA 2.5%) also does not demonstrate a doubling of the mean Cmax or AUCt for bupivacaine. The 15 mL 40K EDLA 1.25% treatment results in a Cmax of 41082.2 ng/mL and an AUCt of 2294113.0 ng/mL·h, while the 15 mL 40K EDLA 2.5% treatment results in a Cmax of 54719.2 ng/mL and an AUCt of 3526532 ng/mL·h.

Therefore, it appears as though the tissue Cmax and AUCt for bupivacaine delivered as 40K EDLA do not demonstrate dose-proportionality over the two doses tested with the current sample size, regardless of whether the dose is increased by increasing the injection volume or the concentration. Although the higher dose does result in a higher tissue bupivacaine Cmax and AUCt, a doubling of the dose does not result in a precise doubling of these endpoints.

The mean tissue tmax for bupivacaine is roughly similar for the three 40K EDLA treatments where the concentration administered was 2.5% (31.6, 33.7, and 32.8 hours), whereas the mean tmax for 40K EDLA given at a 1.25% concentration was approximately half as long (15.0 hours). This would suggest that the tmax is dependent on the concentration of 40K EDLA administered; this occurred despite an identical total 40K EDLA dose for the 7.5 mL 40K EDLA 2.5% and 15 mL 40K EDLA 1.25% treatments. The mean tissue tmax for either AB treatment (0.5 and 1.9 hours) was much shorter than that of any EDLA treatments.

The mean tissue t1/2 for bupivacaine was slightly lower for the two 15 mL 40K EDLA 2.5% treatments (32.6 and 25.0 hours) versus the 7.5 mL 40K EDLA 2.5% treatment (46.3 hours) and the 15 mL 40K EDLA 1.25% treatment (49.1 hours), although there was large variability. The mean tissue t1/2 for bupivacaine delivered as AB (34.0 and 36.4 hours) showed some similarity with the t1/2 of bupivacaine delivered as 40K EDLA.

The mean tissue MRT for bupivacaine was roughly uniform across the different 40K EDLA treatments (42.4, 41.7, 39.1, and 41.0 hours) and was consistently longer than that of either AB treatment (14.6 and 14.2 hours).

The reliability of the microdialysis methodology in measuring local tissue concentrations of dexamethasone was also assessed by determining the similarity of the tissue pharmacokinetic metrics of dexamethasone obtained at different sites using identical doses of 40K EDLA. For the two 15 mL EDLA 2.5% treatments, the mean tissue Cmax (91.5 ng/mL vs. 97.3 ng/mL) and AUCt (2495.8 vs. 2723.6 ng/mL·h) obtained were roughly similar, indicating that the microdialysis method can reliably detect similar peak and total dexamethasone exposure following a similar dose of dexamethasone delivered as 40K EDLA at 2 different sites.

The relationship between 40K EDLA dose and tissue dexamethasone concentration was examined by comparing the tissue pharmacokinetic metrics of dexamethasone obtained at different sites where different doses of 40K EDLA were delivered. Although the 15 mL 40K EDLA 2.5% treatment delivers twice the dose of dexamethasone as the 7.5 mL 40K EDLA 2.5%, the Cmax measured by the assay for the 15 mL 40K EDLA 2.5% treatment was less than double that of the 7.5 mL 40K EDLA 2.5% treatment (58.4 vs. 91.5 ng/mL), although there was large variability. Similarly, the AUCt for the 2 treatments (1831.2 vs 2495.8 ng/mL·h) do not reflect a doubling of total exposure between the two treatments, possibly due to large variability. Since the injection volume differed between the 2 treatments, it is likely that this difference may have affected the total dose of dexamethasone delivered to the tissue.

Comparison of 2 treatments in which one delivers twice the dose of dexamethasone but delivers the same injection volume (15 mL 40K EDLA 1.25% vs. 15 mL 40K EDLA 2.5%) also does not demonstrate a doubling of the Cmax for dexamethasone (62.2 vs. 97.3 ng/mL), although the AUCt between the 2 treatments is roughly double (1396.5 vs. 2723.6 ng/mL·h). Again, the large variability and the relatively small sample size may have contributed to the lack of dose-proportionality.

Therefore, as for bupivacaine, the tissue Cmax and AUCt for dexamethasone do not show acceptable dose-proportionality over the two doses tested, regardless of whether the dose is increased by increasing the injection volume or the concentration. Although the higher dose does result in higher tissue bupivacaine Cmax and AUCt, a doubling of the dose does not consistently result in a doubling of these metrics.

The tmax for dexamethasone was fairly consistent across 40K EDLA treatments (9.1, 7.4, 9.5 hours), except for a value of 95.2 hours that greatly skewed the mean of the 7.5 mL 40K EDLA 2.5% treatment (24.0 hours). The t1/2 was somewhat more consistent across 40K EDLA treatments (20.8, 21.4, 26.9, 19.0 hours), as was the MRT (31.7, 28.3, 25.2, 26.9 hours).

### Pharmacodynamics Results

Analysis of pharmacodynamics results was performed using data from the Evaluable for Efficacy Population.

### Sensory Testing Results Over Time: Part One

Tables J-12(A), J-12(B), J-12(C), and J-12(D) present the results of sensory testing over time in Part 1 for Pin-Prick, Somesthetic, WDT, and HPDT testing, respectively.

TABLE J-12(A)
Pin-Prick Testing Results^{a} Over Time: Part 1
Evaluable for Efficacy Population

| | | | | |
|---|---|---|---|---|
| 15 mL 40K EDLA | 15 mL 40K EDLA | 15 mL 40K EDLA | | |
| 2.5% | 2.5% | 2.5% | 15 mL AB 0.5% | 15 mL AB 0.5% |
| unilateral | right | left | right | left |

Baseline

| | | | | | |
|---|---|---|---|---|---|
| N | 4 | 4 | 4 | 4 | 4 |
| Mean ± SEM | 2.0 ± 0 | 2.0 ± 0 | 2.0 ± 0 | 2.0 ± 0 | 2.0 ± 0 |
| Median | 2.0 | 2.0 | 2.0 | 2.0 | 2.0 |
| Min, Max | 2, 2 | 2, 2 | 2, 2 | 2, 2 | 2, 2 |
| 30 min | | | | | |
| N | 4 | 4 | 4 | 4 | 4 |
| Mean ± SEM | 1.3 ± 0.3 | 1.0 ± 0.4 | 1.0 ± 0.4 | 0.5 ± 0.3 | 0.3 ± 0.3 |
| Median | 1.0 | 1.0 | 1.0 | 0.5 | 0 |
| Min, Max | 1, 2 | 0, 2 | 0, 2 | 0, 1 | 0, 1 |
| 1 h | | | | | |
| N | 4 | 4 | 4 | 4 | 4 |
| Mean ± SEM | 1.5 ± 0.3 | 0.5 ± 0.3 | 1.0 ± 0.4 | 0.8 ± 0.3 | 0.3 ± 0.3 |
| Median | 1.5 | 0.5 | 1.0 | 1.0 | 0 |
| Min, Max | 1, 2 | 0, 1 | 0, 2 | 0, 1 | 0, 1 |
| 3 h | | | | | |
| N | 4 | 4 | 4 | 4 | 4 |
| Mean ± SEM | 1.0 ± 0.4 | 1.0 ± 0.4 | 1.0 ± 0.4 | 0.5 ± 0.3 | 0 ± 0 |
| Median | 1.0 | 1.0 | 1.0 | 0.5 | 0 |
| Min, Max | 0, 2 | 0, 2 | 0, 2 | 0, 1 | 0, 0 |
| 6 h | | | | | |
| N | 4 | 4 | 4 | 4 | 4 |
| Mean ± SEM | 0.8 ± 0.3 | 0.8 ± 0.5 | 0.5 ± 0.3 | 0.8 ± 0.3 | 0.3 ± 0.3 |
| Median | 1.0 | 0.5 | 0.5 | 1.0 | 0 |
| Min, Max | 0, 1 | 0, 2 | 0, 1 | 0, 1 | 0, 1 |
| 12 h | | | | | |
| N | 4 | 4 | 4 | 4 | 4 |
| Mean ± SEM | 0.3 ± 0.3 | 0 ± 0 | 0.8 ± 0.3 | 0.5 ± 0.3 | 0.3 ± 0.3 |
| Median | 0 | 0 | 1.0 | 0.5 | 0 |
| Min, Max | 0, 1 | 0, 0 | 0, 1 | 0, 1 | 0, 1 |
| 24 h | | | | | |
| N | 4 | 4 | 4 | 4 | 4 |
| Mean ± SEM | 0.3 ± 0.3 | 0.5 ± 0.3 | 0.8 ± 0.3 | 1.5 ± 0.3 | 1.0 ± 0 |
| Median | 0 | 0.5 | 1.0 | 1.5 | 1.0 |
| Min, Max | 0, 1 | 0, 1 | 0, 1 | 1, 2 | 1, 1 |
| 48 h | | | | | |
| N | 4 | 4 | 4 | 4 | 4 |
| Mean ± SEM | 0.3 ± 0.3 | 0.3 ± 0.3 | 0 ± 0 | 1.8 ± 0.3 | 2.0 ± 0 |
| Median | 0 | 0 | 0 | 2.0 | 2.0 |
| Min, Max | 0, 1 | 0, 1 | 0, 0 | 1, 2 | 2, 2 |
| 72 h | | | | | |
| N | 4 | 3 | 3 | 4 | 4 |
| Mean ± SEM | 0.3 ± 0.3 | 1.0 ± 0.6 | 0.7 ± 0.3 | 2.0 ± 0 | 1.5 ± 0.3 |
| Median | 0 | 1.0 | 1.0 | 2.0 | 1.5 |
| Min, Max | 0, 1 | 0, 2 | 0, 1 | 2, 2 | 1, 2 |
| 96 h | | | | | |
| N | 4 | 4 | 4 | 4 | 4 |
| Mean ± SEM | 0.3 ± 0.3 | 1.3 ± 0.5 | 1.8 ± 0.3 | 1.8 ± 0.3 | 1.8 ± 0.3 |
| Median | 0 | 1.5 | 2.0 | 2.0 | 2.0 |
| Min, Max | 0, 1 | 0, 2 | 1, 2 | 1, 2 | 1, 2 |

TABLE J-12(B)
Somesthetic Testing Results^{a} Over Time: Part 1
Evaluable for Efficacy Population

| | | | | |
|---|---|---|---|---|
| 15 mL 40K EDLA | 15 mL 40K EDLA | 15 mL 40K EDLA | | |
| 2.5% | 2.5% | 2.5% | 15 mL AB 0.5% | 15 mL AB 0.5% |
| unilateral | right | left | right | left |

Baseline

| | | | | | |
|---|---|---|---|---|---|
| N | 3 | 4 | 4 | 4 | 4 |
| Mean ± SEM | 1.0 ± 0 | 0.8 ± 0.3 | 1.0 ± 0 | 1.0 ± 0 | 1.0 ± 0 |
| Median | 1.0 | 1.0 | 1.0 | 1.0 | 1.0 |
| Min, Max | 1, 1 | 0, 1 | 1, 1 | 1, 1 | 1, 1 |
| 30 min | | | | | |
| N | 4 | 4 | 4 | 4 | 4 |
| Mean ± SEM | 0.3 ± 0.3 | 0.3 ± 0.3 | 0.3 ± 0.3 | 0 ± 0 | 0 ± 0 |
| Median | 0 | 0 | 0 | 0 | 0 |
| Min, Max | 0, 1 | 0, 1 | 0, 1 | 0, 0 | 0, 0 |
| 1 h | | | | | |
| N | 4 | 4 | 4 | 4 | 4 |
| Mean ± SEM | 0 ± 0 | 0.3 ± 0.3 | 0 ± 0 | 0 ± 0 | 0 ± 0 |
| Median | 0 | 0 | 0 | 0 | 0 |
| Min, Max | 0, 0 | 0, 1 | 0, 0 | 0, 0 | 0, 0 |
| 3 h | | | | | |
| N | 4 | 4 | 4 | 4 | 4 |
| Mean ± SEM | 0 ± 0 | 0.3 ± 0.3 | 0.3 ± 0.3 | 0.3 ± 0.3 | 0 ± 0 |
| Median | 0 | 0 | 0 | 0 | 0 |
| Min, Max | 0, 0 | 0, 1 | 0, 1 | 0, 1 | 0, 0 |
| 6 h | | | | | |
| N | 4 | 4 | 4 | 4 | 4 |
| Mean ± SEM | 0 ± 0 | 0 ± 0 | 0 ± 0 | 0 ± 0 | 0 ± 0 |
| Median | 0 | 0 | 0 | 0 | 0 |
| Min, Max | 0, 0 | 0, 0 | 0, 0 | 0, 0 | 0, 0 |
| 12 h | | | | | |
| N | 4 | 4 | 4 | 4 | 4 |
| Mean ± SEM | 0.3 ± 0.3 | 0.3 ± 0.3 | 0.3 ± 0.3 | 0 ± 0 | 0 ± 0 |
| Median | 0 | 0 | 0 | 0 | 0 |
| Min, Max | 0, 1 | 0, 1 | 0, 1 | 0, 0 | 0, 0 |
| 24 h | | | | | |
| N | 4 | 4 | 4 | 4 | 4 |
| Mean ± SEM | 0.3 ± 0.3 | 0.5 ± 0.3 | 0.3 ± 0.3 | 0.5 ± 0.3 | 0 ± 0 |
| Median | 0 | 0.5 | 0 | 0.5 | 0 |
| Min, Max | 0, 1 | 0, 1 | 0, 1 | 0, 1 | 0, 0 |
| 48 h | | | | | |
| N | 4 | 4 | 4 | 4 | 4 |
| Mean ± SEM | 0.3 ± 0.3 | 0.3 ± 0.3 | 0 ± 0 | 1.0 ± 0 | 0.5 ± 0.3 |
| Median | 0 | 0 | 0 | 1.0 | 0.5 |
| Min, Max | 0, 1 | 0, 1 | 0, 0 | 1, 1 | 0, 1 |
| 72 h | | | | | |
| N | 4 | 3 | 3 | 4 | 4 |
| Mean ± SEM | 0 ± 0 | 0 ± 0 | 0.3 ± 0.3 | 1.0 ± 0 | 0.5 ± 0.3 |
| Median | 0 | 0 | 0 | 1.0 | 0.5 |
| Min, Max | 0, 0 | 0, 0 | 0, 1 | 1, 1 | 0, 1 |
| 96 h | | | | | |
| N | 4 | 4 | 4 | 4 | 4 |
| Mean ± SEM | 0.3 ± 0.3 | 0.5 ± 0.3 | 0.3 ± 0.3 | 1.0 ± 0 | 1.0 ± 0 |
| Median | 0 | 0.5 | 0 | 1.0 | 1.0 |
| Min, Max | 0, 1 | 0, 1 | 0, 1 | 1, 1 | 1, 1 |

TABLE J-12(C)
Warmth Detection Threshold Testing Results^{a} (° C.) Over Time: Part 1
Evaluable for Efficacy Population

| | | | | |
|---|---|---|---|---|
| 15 mL 40K EDLA | 15 mL 40K EDLA | 15 mL 40K EDLA | | |
| 2.5% | 2.5% | 2.5% | 15 mL AB 0.5% | 15 mL AB 0.5% |
| unilateral | right | left | right | left |

Baseline

| | | | | | |
|---|---|---|---|---|---|
| n | 4 | 4 | 4 | 4 | 4 |
| Mean ± SEM | 40.1 ± 2.6 | 44.3 ± 1.3 | 43.6 ± 1.2 | 43.2 ± 1.2 | 40.9 ± 1.0 |
| Median | 42.6 | 43.5 | 43.7 | 42.1 | 40.8 |
| Min, Max | 32.2, 42.9 | 42.2, 47.9 | 40.6, 46.3 | 41.6, 46.8 | 38.4, 43.5 |
| 30 min | | | | | |
| n | 4 | 3 | 3 | 3 | 3 |
| Mean ± SEM | 47.1 ± 1.2 | 50.1 ± 0.4 | 48.2 ± 0.2 | 50.4 ± 0.6 | 51.0 ± 0 |
| Median | 47.3 | 49.8 | 48.2 | 51.0 | 51.0 |
| Min, Max | 44.6, 49.1 | 49.6, 51.0 | 47.8, 48.5 | 49.3, 51.0 | 51.0, 51.0 |
| 1 h | | | | | |
| n | 4 | 4 | 4 | 4 | 4 |
| Mean ± SEM | 46.8 ± 1.7 | 50.0 ± 0.6 | 50.0 ± 0.9 | 49.8 ± 0.7 | 49.2 ± 1.8 |
| Median | 46.7 | 50.1 | 50.8 | 50.2 | 51.0 |
| Min, Max | 42.8, 51.0 | 48.6, 51.0 | 47.3, 51.0 | 47.9, 51.0 | 43.7, 51.0 |
| 3 h | | | | | |
| n | 4 | 4 | 4 | 4 | 4 |
| Mean ± SEM | 47.5 ± 0.4 | 51.0 ± 0 | 49.3 ± 1.7 | 50.3 ± 0.7 | 49.6 ± 1.5 |
| Median | 47.4 | 51.0 | 51.0 | 51.0 | 51.0 |
| Min, Max | 46.6, 48.7 | 51.0, 51.0 | 44.1, 51.0 | 48.1, 51.0 | 45.2, 51.0 |
| 6 h | | | | | |
| n | 4 | 4 | 4 | 4 | 4 |
| Mean ± SEM | 46.8 ± 1.8 | 51.0 ± 0 | 47.8 ± 1.6 | 49.8 ± 1.3 | 49.8 ± 1.2 |
| Median | 46.2 | 51.0 | 47.8 | 51.0 | 51.0 |
| Min, Max | 43.7, 51.0 | 51.0, 51.0 | 44.5, 51.0 | 46.0, 51.0 | 46.2, 51.0 |
| 12 h | | | | | |
| n | 4 | 4 | 4 | 4 | 4 |
| Mean ± SEM | 48.5 ± 2.0 | 50.5 ± 0.5 | 50.1 ± 0.7 | 50.0 ± 1.0 | 49.7 ± 1.4 |
| Median | 50.2 | 51.0 | 50.5 | 51.0 | 51.0 |
| Min, Max | 42.5, 51.0 | 49.1, 51.0 | 48.2, 51.0 | 47.1, 51.0 | 45.6, 51.0 |
| 24 h | | | | | |
| n | 4 | 4 | 4 | 4 | 4 |
| Mean ± SEM | 46.3 ± 1.8 | 51.0 ± 0 | 50.2 ± 0.8 | 45.3 ± 2.2 | 43.2 ± 2.3 |
| Median | 46.3 | 51.0 | 51.0 | 44.9 | 42.5 |
| Min, Max | 42.5, 50.0 | 51.0, 51.0 | 47.8, 51.0 | 40.2, 51.0 | 38.5, 49.3 |
| 48 h | | | | | |
| n | 4 | 4 | 4 | 4 | 4 |
| Mean ± SEM | 44.8 ± 4.5 | 48.5 ± 2.5 | 47.9 ± 2.0 | 38.7 ± 3.7 | 40.8 ± 3.0 |
| Median | 48.2 | 51.0 | 49.1 | 37.8 | 38.9 |
| Min, Max | 31.8, 51.0 | 40.9, 51.0 | 42.5, 51.0 | 31.4, 47.7 | 36.3, 49.0 |
| 72 h | | | | | |
| n | 4 | 3 | 3 | 4 | 4 |
| Mean ± SEM | 45.6 ± 0.3 | 44.6 ± 4.4 | 45.8 ± 2.3 | 39.2 ± 1.1 | 39.4 ± 0.7 |
| Median | 45.5 | 46.6 | 45.5 | 39.8 | 39.9 |
| Min, Max | 45.1, 46.2 | 36.2, 51.0 | 41.9, 50.0 | 36.1, 41.2 | 37.5, 40.4 |
| 96 h | | | | | |
| n | 4 | 4 | 4 | 4 | 4 |
| Mean ± SEM | 46.2 ± 2.4 | 45.8 ± 1.2 | 46.5 ± 1.8 | 40.5 ± 1.8 | 40.0 ± 2.3 |
| Median | 46.7 | 45.5 | 46.1 | 40.2 | 41.8 |
| Min, Max | 40.5, 51.0 | 43.4, 48.7 | 42.9, 51.0 | 37.1, 44.3 | 33.3, 43.1 |

TABLE J-12(D)
Heat Pain Detection Threshold Testing Results^{a} (° C.) Over Time: Part 1
Evaluable for Efficacy Population

| | | | | |
|---|---|---|---|---|
| 15 mL 40K EDLA | 15 mL 40K EDLA | 15 mL 40K EDLA | | |
| 2.5% | 2.5% | 2.5% | 15 mL AB 0.5% | 15 mL AB 0.5% |
| unilateral | right | left | right | left |

Baseline

| | | | | | |
|---|---|---|---|---|---|
| n | 4 | 4 | 4 | 4 | 4 |
| Mean ± SEM | 46.1 ± 0.3 | 49.7 ± 0.9 | 48.7 ± 0.9 | 48.9 ± 1.6 | 47.6 ± 1.2 |
| Median | 45.9 | 50.3 | 48.6 | 50.3 | 48.5 |
| Min, Max | 45.7, 46.8 | 47.2, 51.0 | 46.8, 51.0 | 44.1, 51.0 | 44.2, 49.4 |
| 30 min | | | | | |
| n | 4 | 3 | 3 | 3 | 3 |
| Mean ± SEM | 50.7 ± 0.3 | 50.7 ± 0.3 | 50.0 ± 0.6 | 50.4 ± 0.6 | 51.0 ± 0 |
| Median | 51.0 | 51.0 | 50.2 | 51.0 | 51.0 |
| Min, Max | 49.8, 51.0 | 50.1, 51.0 | 48.9, 51.0 | 49.3, 51.0 | 51.0, 51.0 |
| 1 h | | | | | |
| n | 4 | 4 | 4 | 4 | 4 |
| Mean ± SEM | 50.3 ± 0.5 | 51.0 ± 0 | 50.7 ± 0.3 | 49.8 ± 0.7 | 49.2 ± 1.8 |
| Median | 50.6 | 51.0 | 51.0 | 50.2 | 51.0 |
| Min, Max | 49.0, 51.0 | 51.0, 51.0 | 49.9, 51.0 | 47.9, 51.0 | 43.7, 51.0 |
| 3 h | | | | | |
| n | 4 | 4 | 4 | 4 | 4 |
| Mean ± SEM | 49.3 ± 0.7 | 51.0 ± 0 | 50.6 ± 0.4 | 50.3 ± 0.7 | 49.6 ± 1.5 |
| Median | 49.0 | 51.0 | 51.0 | 51.0 | 51.0 |
| Min, Max | 48.2, 51.0 | 51.0, 51.0 | 49.3, 51.0 | 48.1, 51.0 | 45.2, 51.0 |
| 6 h | | | | | |
| N | 4 | 4 | 4 | 4 | 4 |
| Mean ± SEM | 48.2 ± 0.9 | 51.0 ± 0 | 49.9 ± 0.8 | 49.8 ± 1.3 | 50.8 ± 0.2 |
| Median | 48.7 | 51.0 | 50.4 | 51.0 | 51.0 |
| Min, Max | 45.6, 49.9 | 51.0, 51.0 | 47.6, 51.0 | 46.0, 51.0 | 50.1, 51.0 |
| 12 h | | | | | |
| N | 4 | 4 | 4 | 4 | 4 |
| Mean ± SEM | 50.0 ± 0.7 | 51.0 ± 0 | 51.0 ± 0.1 | 50.1 ± 1.0 | 50.0 ± 1.0 |
| Median | 50.3 | 51.0 | 51.0 | 51.0 | 51.0 |
| Min, Max | 48.2, 51.0 | 51.0, 51.0 | 50.8, 51.0 | 47.2, 51.0 | 47.0, 51.0 |
| 24 h | | | | | |
| n | 4 | 4 | 4 | 4 | 4 |
| Mean ± SEM | 48.3 ± 1.1 | 51.0 ± 0 | 50.7 ± 0.4 | 46.8 ± 2.3 | 45.4 ± 2.4 |
| Median | 48.6 | 51.0 | 51.0 | 47.9 | 46.9 |
| Min, Max | 45.6, 50.3 | 51.0, 51.0 | 49.6, 51.0 | 40.2, 51.0 | 38.5, 49.3 |
| 48 h | | | | | |
| n | 4 | 4 | 4 | 4 | 4 |
| Mean ± SEM | 47.6 ± 2.8 | 49.7 ± 1.4 | 50.4 ± 0.4 | 42.7 ± 3.6 | 43.7 ± 2.8 |
| Median | 50.1 | 51.0 | 50.7 | 43.2 | 43.8 |
| Min, Max | 39.2, 51.0 | 45.6, 51.0 | 49.1, 51.0 | 35.3, 49.1 | 37.4, 49.8 |
| 72 h | | | | | |
| n | 4 | 3 | 3 | 4 | 4 |
| Mean ± SEM | 46.7 ± 0.5 | 46.0 ± 4.0 | 47.8 ± 2.4 | 43.9 ± 1.3 | 43.7 ± 1.5 |
| Median | 47.2 | 48.8 | 49.2 | 44.0 | 44.0 |
| Min, Max | 45.1, 47.5 | 38.1, 51.0 | 43.2, 51.0 | 40.9, 46.9 | 40.1, 46.8 |
| 96 h | | | | | |
| n | 4 | 4 | 4 | 4 | 4 |
| Mean ± SEM | 48.0 ± 1.5 | 47.1 ± 1.4 | 48.2 ± 1.1 | 45.6 ± 2.6 | 44.3 ± 3.4 |
| Median | 48.5 | 46.6 | 48.1 | 47.5 | 46.7 |
| Min, Max | 44.0, 51.0 | 44.4, 50.8 | 45.7, 51.0 | 38.0, 49.4 | 34.3, 49.4 |

### Sensory Testing Results Over Time: Part Two

Tables J-13(A), J-13(B), J-13(C), and J-13(D) present the results of sensory testing over time in Part 2 for Pin-Prick, Somesthetic, WDT, and HPDT testing, respectively.

TABLE J-13(A)
Pin-Prick Testing Results^{a} Over Time: Part 2
Evaluable for Efficacy Population

| | | | | | |
|---|---|---|---|---|---|
| 7.5 mL 40K | 15 mL 40K | 15 mL 40K | 15 mL 40K | 15 mL AB | |
| EDLA 2.5% | EDLA 2.5% | EDLA 1.25% | EDLA 2.5% | 0.25% | 15 mL AB 0.25% |
| right | left | right | left | right | left |
| (n = 6) | (n = 6) | (n = 5) | (n = 5) | (n = 4) | (n = 4) |

Baseline

| | | | | | | |
|---|---|---|---|---|---|---|
| n | 6 | 6 | 5 | 5 | 4 | 4 |
| Mean ± SEM | 2.0 ± 0 | 2.0 ± 0 | 2.0 ± 0 | 2.0 ± 0 | 2.0 ± 0 | 2.0 ± 0 |
| Median | 2.0 | 2.0 | 2.0 | 2.0 | 2.0 | 2.0 |
| Min, Max | 2, 2 | 2, 2 | 2, 2 | 2, 2 | 2, 2 | 2, 2 |
| 30 min | | | | | | |
| n | 6 | 6 | 5 | 5 | 4 | 4 |
| Mean ± SEM | 1.3 ± 0.2 | 1.3 ± 0.2 | 1.6 ± 0.2 | 1.0 ± 0 | 1.0 ± 0 | 1.0 ± 0 |
| Median | 1.0 | 1.0 | 2.0 | 1.0 | 1.0 | 1.0 |
| Min, Max | 1, 2 | 1, 2 | 1, 2 | 1, 1 | 1, 1 | 1, 1 |
| 1 h | | | | | | |
| n | 6 | 6 | 5 | 5 | 4 | 4 |
| Mean ± SEM | 1.2 ± 0.2 | 1.0 ± 0.3 | 0.8 ± 0.2 | 1.2 ± 0.2 | 0.8 ± 0.3 | 0.8 ± 0.3 |
| Median | 1.0 | 1.0 | 1.0 | 1.0 | 1.0 | 1.0 |
| Min, Max | 1, 2 | 0, 2 | 0, 1 | 1, 2 | 0, 1 | 0, 1 |
| 3 h | | | | | | |
| n | 6 | 6 | 5 | 5 | 4 | 4 |
| Mean ± SEM | 0.8 ± 0.2 | 0.7 ± 0.2 | 1.0 ± 0.3 | 0.8 ± 0.2 | 1.0 ± 0 | 1.0 ± 0 |
| Median | 1.0 | 1.0 | 1.0 | 1.0 | 1.0 | 1.0 |
| Min, Max | 0, 1 | 0, 1 | 0, 2 | 0, 1 | 1, 1 | 1, 1 |
| 6 h | | | | | | |
| n | 6 | 6 | 5 | 5 | 4 | 4 |
| Mean ± SEM | 0.8 ± 0.2 | 0.5 ± 0.2 | 1.2 ± 0.4 | 1.2 ± 0.2 | 0.8 ± 0.3 | 0.8 ± 0.3 |
| Median | 1.0 | 0.5 | 1.0 | 1.0 | 1.0 | 1.0 |
| Min, Max | 0, 1 | 0, 1 | 0, 2 | 1, 2 | 0, 1 | 0, 1 |
| 12 h | | | | | | |
| n | 6 | 6 | 5 | 5 | 4 | 4 |
| Mean ± SEM | 0.7 ± 0.2 | 0.5 ± 0.2 | 0.8 ± 0.2 | 1.2 ± 0.2 | 1.3 ± 0.3 | 1.3 ± 0.3 |
| Median | 1.0 | 0.5 | 1.0 | 1.0 | 1.0 | 1.0 |
| Min, Max | 0, 1 | 0, 1 | 0, 1 | 1, 2 | 1, 2 | 1, 2 |
| 24 h | | | | | | |
| n | 6 | 6 | 5 | 5 | 4 | 4 |
| Mean ± SEM | 0.8 ± 0.2 | 0.5 ± 0.2 | 0.8 ± 0.2 | 0.8 ± 0.2 | 1.3 ± 0.3 | 1.3 ± 0.3 |
| Median | 1.0 | 0.5 | 1.0 | 1.0 | 1.0 | 1.0 |
| Min, Max | 0, 1 | 0, 1 | 0, 1 | 0, 1 | 1, 2 | 1, 2 |
| 48 h | | | | | | |
| n | 6 | 6 | 5 | 5 | 4 | 4 |
| Mean ± SEM | 0.7 ± 0.2 | 0.7 ± 0.2 | 0.8 ± 0.2 | 0.4 ± 0.2 | 1.8 ± 0.3 | 1.8 ± 0.3 |
| Median | 1.0 | 1.0 | 1.0 | 0 | 2.0 | 2.0 |
| Min, Max | 0, 1 | 0, 1 | 0, 1 | 0, 1 | 1, 2 | 1, 2 |
| 72 h | | | | | | |
| n | 6 | 6 | 5 | 5 | 4 | 4 |
| Mean ± SEM | 0.8 ± 0.2 | 0.7 ± 0.2 | 0.8 ± 0.4 | 0.8 ± 0.2 | 2.0 ± 0 | 2.0 ± 0 |
| Median | 1.0 | 1.0 | 1.0 | 1.0 | 2.0 | 2.0 |
| Min, Max | 0, 1 | 0, 1 | 0, 2 | 0, 1 | 2, 2 | 2, 2 |
| 96 h | | | | | | |
| n | 6 | 6 | 5 | 5 | 4 | 4 |
| Mean ± SEM | 1.2 ± 0.2 | 0.8 ± 0.2 | 1.6 ± 0.2 | 1.4 ± 0.2 | 2.0 ± 0 | 2.0 ± 0 |
| Median | 1.0 | 1.0 | 2.0 | 1.0 | 2.0 | 2.0 |
| Min, Max | 1, 2 | 0, 1 | 1, 2 | 1, 2 | 2, 2 | 2, 2 |

TABLE J-13(B)
Somesthetic Testing Results^{a} Over Time: Part 2
Evaluable for Efficacy Population

| | | | | | |
|---|---|---|---|---|---|
| 7.5 mL 40K | 15 mL 40K | 15 mL 40K | 15 mL 40K | 15 mL AB | |
| EDLA 2.5% | EDLA 2.5% | EDLA 1.25% | EDLA 2.5% | 0.25% | 15 mL AB 0.25% |
| right | left | right | left | right | left |
| (n = 6) | (n = 6) | (n = 5) | (n = 5) | (n = 4) | (n = 4) |

Baseline

| | | | | | | |
|---|---|---|---|---|---|---|
| n | 6 | 6 | 5 | 5 | 4 | 4 |
| Mean ± SEM | 0.5 ± 0.2 | 0.5 ± 0.2 | 1.0 ± 0 | 1.0 ± 0 | 1.0 ± 0 | 1.0 ± 0 |
| Median | 0.5 | 0.5 | 1.0 | 1.0 | 1.0 | 1.0 |
| Min, Max | 0, 1 | 0, 1 | 1, 1 | 1, 1 | 1, 1 | 1, 1 |
| 30 min | | | | | | |
| n | 6 | 6 | 5 | 5 | 4 | 4 |
| Mean ± SEM | 0.3 ± 0.2 | 0.2 ± 0.2 | 0.8 ± 0.2 | 0.6 ± 0.2 | 0 ± 0 | 0 ± 0 |
| Median | 0 | 0 | 1.0 | 1.0 | 0 | 0 |
| Min, Max | 0, 1 | 0, 1 | 0, 1 | 0, 1 | 0, 0 | 0, 0 |
| 1 h | | | | | | |
| n | 6 | 6 | 5 | 5 | 4 | 4 |
| Mean ± SEM | 0.3 ± 0.2 | 0.2 ± 0.2 | 0.4 ± 0.2 | 0 ± 0 | 0 ± 0 | 0 ± 0 |
| Median | 0 | 0 | 0 | 0 | 0 | 0 |
| Min, Max | 0, 1 | 0, 1 | 0, 1 | 0, 0 | 0, 0 | 0, 0 |
| 3 h | | | | | | |
| n | 6 | 6 | 5 | 5 | 4 | 4 |
| Mean ± SEM | 0 ± 0 | 0 ± 0 | 0.2 ± 0.2 | 0 ± 0 | 0 ± 0 | 0 ± 0 |
| Median | 0 | 0 | 0 | 0 | 0 | 0 |
| Min, Max | 0, 0 | 0, 0 | 0, 1 | 0, 0 | 0, 0 | 0, 0 |
| 6 h | | | | | | |
| n | 6 | 6 | 5 | 5 | 4 | 4 |
| Mean ± SEM | 0.2 ± 0.2 | 0.2 ± 0.2 | 0 ± 0 | 0 ± 0 | 0 ± 0 | 0 ± 0 |
| Median | 0 | 0 | 0 | 0 | 0 | 0 |
| Min, Max | 0, 1 | 0, 1 | 0, 0 | 0, 0 | 0, 0 | 0, 0 |
| 12 h | | | | | | |
| n | 6 | 6 | 5 | 5 | 4 | 4 |
| Mean ± SEM | 0 ± 0 | 0 ± 0 | 0 ± 0 | 0.2 ± 0.2 | 0 ± 0 | 0.3 ± 0.3 |
| Median | 0 | 0 | 0 | 0 | 0 | 0 |
| Min, Max | 0, 0 | 0, 0 | 0, 0 | 0, 1 | 0, 0 | 0, 1 |
| 24 h | | | | | | |
| n | 6 | 6 | 5 | 5 | 4 | 4 |
| Mean ± SEM | 0.2 ± 0.2 | 0.2 ± 0.2 | 0 ± 0 | 0 ± 0 | 0.3 ± 0.3 | 0.5 ± 0.3 |
| Median | 0 | 0 | 0 | 0 | 0 | 0.5 |
| Min, Max | 0, 1 | 0, 1 | 0, 0 | 0, 0 | 0, 1 | 0, 1 |
| 48 h | | | | | | |
| n | 6 | 6 | 5 | 5 | 4 | 4 |
| Mean ± SEM | 0.3 ± 0.2 | 0.2 ± 0.2 | 0 ± 0 | 0 ± 0 | 0.5 ± 0.3 | 0.8 ± 0.3 |
| Median | 0 | 0 | 0 | 0 | 0.5 | 1.0 |
| Min, Max | 0, 1 | 0, 1 | 0, 0 | 0, 0 | 0, 1 | 0, 1 |
| 72 h | | | | | | |
| n | 6 | 6 | 5 | 5 | 4 | 4 |
| Mean ± SEM | 0.5 ± 0.2 | 0 ± 0 | 0 ± 0 | 0 ± 0 | 0.8 ± 0.3 | 1.0 ± 0 |
| Median | 0.5 | 0 | 0 | 0 | 1.0 | 1.0 |
| Min, Max | 0, 1 | 0, 0 | 0, 0 | 0, 0 | 0, 1 | 1, 1 |
| 96 h | | | | | | |
| n | 6 | 6 | 5 | 5 | 4 | 4 |
| Mean ± SEM | 0.5 ± 0.2 | 0.3 ± 0.2 | 0.2 ± 0.2 | 0 ± 0 | 1.0 ± 0 | 1.0 ± 0 |
| Median | 0.5 | 0 | 0 | 0 | 1.0 | 1.0 |
| Min, Max | 0, 1 | 0, 1 | 0, 1 | 0, 0 | 1, 1 | 1, 1 |

TABLE J-13(C)
WDT Testing Results^{a} (° C.) Over Time: Part 2
Evaluable for Efficacy Population

| | | | | | |
|---|---|---|---|---|---|
| 7.5 mL 40K | 15 mL 40K | 15 mL 40K | 15 mL 40K | 15 mL AB | |
| EDLA 2.5% | EDLA 2.5% | EDLA 1.25% | EDLA 2.5% | 0.25% | 15 mL AB 0.25% |
| right | left | right | left | right | left |
| (n = 6) | (n = 6) | (n = 5) | (n = 5) | (n = 4) | (n = 4) |

Baseline

| | | | | | | |
|---|---|---|---|---|---|---|
| n | 6 | 6 | 5 | 5 | 4 | 4 |
| Mean ± SEM | 40.6 ± 1.2 | 39.7 ± 1.0 | 37.0 ± 1.5 | 37.1 ± 1.2 | 37.9 ± 2.1 | 38.7 ± 1.4 |
| Median | 41.7 | 40.1 | 37.0 | 35.8 | 38.1 | 38.6 |
| Min, Max | 36.7, 43.5 | 36.1, 42.3 | 34.0, 42.0 | 34.1, 40.5 | 32.6, 42.8 | 35.7, 42.0 |
| 30 min | | | | | | |
| n | 6 | 6 | 5 | 5 | 4 | 4 |
| Mean ± SEM | 45.6 ± 1.2 | 44.2 ± 1.1 | 44.6 ± 1.7 | 45.7 ± 2.1 | 47.8 ± 1.9 | 48.4 ± 0.9 |
| Median | 45.1 | 44.9 | 45.9 | 48.1 | 48.1 | 47.9 |
| Min, Max | 42.0, 49.5 | 40.7, 47.7 | 40.4, 49.1 | 37.9, 49.7 | 43.9, 51.0 | 46.6, 51.0 |
| 1 h | | | | | | |
| n | 6 | 6 | 5 | 5 | 4 | 4 |
| Mean ± SEM | 45.9 ± 1.0 | 43.4 ± 1.7 | 43.2 ± 1.9 | 46.0 ± 1.8 | 49.6 ± 0.8 | 45.5 ± 1.7 |
| Median | 46.0 | 43.9 | 45.1 | 45.8 | 49.9 | 46.1 |
| Min, Max | 42.6, 49.8 | 38.9, 48.3 | 35.9, 46.7 | 40.2, 51.0 | 47.8, 51.0 | 40.8, 49.1 |
| 3 h | | | | | | |
| n | 6 | 6 | 5 | 5 | 4 | 4 |
| Mean ± SEM | 44.8 ± 0.8 | 46.0 ± 1.7 | 45.3 ± 1.9 | 47.1 ± 1.3 | 47.4 ± 1.8 | 45.3 ± 1.8 |
| Median | 45.1 | 44.8 | 47.6 | 47.3 | 47.2 | 45.0 |
| Min, Max | 42.4, 47.4 | 42.0, 51.0 | 40.1, 49.3 | 42.6, 49.9 | 44.0, 51.0 | 41.6, 49.5 |
| 6 h | | | | | | |
| n | 6 | 6 | 5 | 5 | 4 | 4 |
| Mean ± SEM | 47.1 ± 1.1 | 47.7 ± 1.0 | 44.7 ± 2.3 | 46.0 ± 2.1 | 44.4 ± 1.5 | 44.6 ± 1.5 |
| Median | 47.1 | 47.7 | 44.8 | 44.4 | 45.3 | 44.7 |
| Min, Max | 43.9, 51.0 | 44.3, 51.0 | 39.3, 51.0 | 41.0, 51.0 | 40.2, 46.8 | 40.9, 48.1 |
| 12 h | | | | | | |
| n | 5 | 5 | 4 | 4 | 4 | 4 |
| Mean ± SEM | 47.7 ± 1.5 | 48.5 ± 1.5 | 46.1 ± 2.5 | 50.2 ± 0.8 | 47.7 ± 0.6 | 50.2 ± 0.8 |
| Median | 47.6 | 51.0 | 48.4 | 51.0 | 47.6 | 51.0 |
| Min, Max | 43.5, 51.0 | 44.5, 51.0 | 38.7, 49.0 | 47.8, 51.0 | 46.6, 48.9 | 47.9, 51.0 |
| 24 h | | | | | | |
| n | 6 | 6 | 5 | 5 | 4 | 4 |
| Mean ± SEM | 44.9 ± 1.5 | 46.3 ± 2.5 | 48.4 ± 1.2 | 49.8 ± 0.7 | 43.2 ± 1.2 | 42.4 ± 1.1 |
| Median | 43.6 | 48.8 | 47.7 | 51.0 | 43.1 | 43.0 |
| Min, Max | 41.6, 51.0 | 36.2, 51.0 | 44.7, 51.0 | 47.9, 51.0 | 40.6, 46.1 | 39.4, 44.1 |
| 48 h | | | | | | |
| n | 6 | 6 | 5 | 5 | 4 | 4 |
| Mean ± SEM | 45.4 ± 1.7 | 45.9 ± 1.9 | 46.2 ± 1.1 | 49.0 ± 1.1 | 39.6 ± 1.0 | 41.4 ± 2.1 |
| Median | 46.0 | 46.5 | 46.6 | 49.4 | 39.1 | 42.6 |
| Min, Max | 38.6, 51.0 | 37.1, 51.0 | 42.5, 48.8 | 44.9, 51.0 | 37.8, 42.4 | 35.7, 44.6 |
| 72 h | | | | | | |
| n | 6 | 6 | 5 | 5 | 4 | 4 |
| Mean ± SEM | 44.3 ± 1.7 | 45.6 ± 1.2 | 44.6 ± 1.2 | 45.2 ± 1.7 | 40.4 ± 3.1 | 40.8 ± 1.0 |
| Median | 43.8 | 45.3 | 44.5 | 46.3 | 41.6 | 40.6 |
| Min, Max | 37.8, 49.6 | 42.0, 49.9 | 40.7, 48.4 | 40.4, 48.9 | 31.9, 46.5 | 38.8, 43.0 |
| 96 h | | | | | | |
| n | 6 | 6 | 5 | 5 | 4 | 4 |
| Mean ± SEM | 41.6 ± 1.3 | 40.6 ± 0.8 | 43.2 ± 2.4 | 44.8 ± 2.0 | 38.0 ± 2.1 | 38.3 ± 2.1 |
| Median | 42.6 | 40.8 | 40.7 | 46.2 | 38.0 | 37.0 |
| Min, Max | 35.9, 44.5 | 38.3, 42.6 | 38.1, 51.0 | 37.5, 49.6 | 34.2, 41.8 | 35.2, 44.2 |

TABLE J-13(D)
HPDT Testing Results^{a} (° C.) Over Time: Part 2
Evaluable for Efficacy Population

| | | | | | |
|---|---|---|---|---|---|
| 7.5 mL 40K | 15 mL 40K | 15 mL 40K | 15 mL 40K | 15 mL AB | |
| EDLA 2.5% | EDLA 2.5% | EDLA 1.25% | EDLA 2.5% | 0.25% | 15 mL AB 0.25% |
| right | left | right | left | right | left |
| (n = 6) | (n = 6) | (n = 5) | (n = 5) | (n = 4) | (n = 4) |

Baseline

| | | | | | | |
|---|---|---|---|---|---|---|
| N | 6 | 6 | 5 | 5 | 4 | 4 |
| Mean ± SEM | 44.5 ± 1.3 | 44.0 ± 1.1 | 41.8 ± 1.4 | 42.4 ± 1.3 | 43.1 ± 0.8 | 43.0 ± 0.7 |
| Median | 45.7 | 45.1 | 42.7 | 42.6 | 42.9 | 43.5 |
| Min, Max | 38.7, 46.9 | 38.5, 45.7 | 37.6, 45.6 | 39.2, 45.3 | 41.5, 45.0 | 41.0, 43.8 |
| 30 min | | | | | | |
| N | 6 | 6 | 5 | 5 | 4 | 4 |
| Mean ± SEM | 46.8 ± 0.9 | 47.4 ± 1.2 | 46.6 ± 0.9 | 47.3 ± 1.4 | 50.7 ± 0.3 | 50.0 ± 0.6 |
| Median | 46.1 | 48.3 | 46.5 | 48.2 | 51.0 | 50.1 |
| Min, Max | 44.5, 49.8 | 43.2, 50.2 | 44.5, 49.1 | 43.3, 51.0 | 49.7, 51.0 | 48.8, 51.0 |
| 1 h | | | | | | |
| N | 6 | 6 | 5 | 5 | 4 | 4 |
| Mean ± SEM | 47.8 ± 1.0 | 46.5 ± 1.0 | 46.4 ± 1.0 | 47.1 ± 1.4 | 51.0 ± 0 | 49.3 ± 0.6 |
| Median | 47.6 | 46.5 | 46.4 | 47.8 | 51.0 | 49.1 |
| Min, Max | 44.7, 51.0 | 43.3, 51.0 | 42.9, 49.1 | 42.8, 51.0 | 51.0, 51.0 | 47.9, 51.0 |
| 3 h | | | | | | |
| N | 6 | 6 | 5 | 5 | 4 | 4 |
| Mean ± SEM | 47.5 ± 0.9 | 47.9 ± 1.2 | 47.2 ± 1.2 | 48.2 ± 1.0 | 49.3 ± 1.0 | 49.3 ± 1.0 |
| Median | 47.2 | 47.9 | 47.6 | 47.3 | 49.4 | 49.4 |
| Min, Max | 44.6, 51.0 | 44.4, 51.0 | 43.8, 51.0 | 45.9, 51.0 | 47.5, 51.0 | 47.2, 51.0 |
| 6 h | | | | | | |
| N | 6 | 6 | 5 | 5 | 4 | 4 |
| Mean ± SEM | 49.0 ± 0.8 | 48.6 ± 0.6 | 47.7 ± 1.7 | 47.1 ± 1.8 | 48.0 ± 2.1 | 47.9 ± 1.7 |
| Median | 49.1 | 48.0 | 49.2 | 47.6 | 49.4 | 48.6 |
| Min, Max | 46.2, 51.0 | 47.1, 51.0 | 43.3, 51.0 | 42.9, 51.0 | 42.3, 51.0 | 43.4, 51.0 |
| 12 h | | | | | | |
| N | 5 | 5 | 4 | 4 | 4 | 4 |
| Mean ± SEM | 49.5 ± 0.9 | 50.1 ± 0.9 | 46.8 ± 1.8 | 49.4 ± 1.6 | 49.6 ± 0.5 | 50.2 ± 0.8 |
| Median | 51.0 | 51.0 | 48.4 | 51.0 | 49.3 | 51.0 |
| Min, Max | 46.9, 51.0 | 46.4, 51.0 | 41.4, 49.0 | 44.5, 51.0 | 48.7, 51.0 | 47.9, 51.0 |
| 24 h | | | | | | |
| N | 6 | 6 | 5 | 5 | 4 | 4 |
| Mean ± SEM | 46.3 ± 1.4 | 46.9 ± 2.2 | 49.2 ± 0.8 | 50.4 ± 0.6 | 46.0 ± 0.9 | 46.1 ± 0.4 |
| Median | 46.5 | 49.1 | 48.8 | 51.0 | 45.7 | 46.0 |
| Min, Max | 40.9, 51.0 | 37.8, 51.0 | 47.4, 51.0 | 47.9, 51.0 | 44.1, 48.4 | 45.2, 47.0 |
| 48 h | | | | | | |
| N | 6 | 6 | 5 | 5 | 4 | 4 |
| Mean ± SEM | 47.5 ± 1.2 | 48.3 ± 1.5 | 47.7 ± 0.9 | 50.3 ± 0.5 | 44.3 ± 0.7 | 44.9 ± 0.5 |
| Median | 47.0 | 49.1 | 47.0 | 51.0 | 44.3 | 44.7 |
| Min, Max | 42.8, 51.0 | 41.1, 51.0 | 46.0, 51.0 | 48.9, 51.0 | 43.0, 45.7 | 43.9, 46.3 |
| 72 h | | | | | | |
| N | 6 | 6 | 5 | 5 | 4 | 4 |
| Mean ± SEM | 47.3 ± 1.6 | 47.8 ± 1.3 | 46.1 ± 0.9 | 47.4 ± 1.1 | 44.4 ± 1.8 | 44.6 ± 0.8 |
| Median | 47.5 | 48.1 | 45.9 | 46.8 | 44.8 | 44.8 |
| Min, Max | 40.7, 51.0 | 43.4, 51.0 | 43.5, 48.4 | 44.2, 50.6 | 39.9, 48.1 | 42.4, 46.2 |
| 96 h | | | | | | |
| N | 6 | 6 | 5 | 5 | 4 | 4 |
| Mean ± SEM | 43.9 ± 1.4 | 44.9 ± 1.2 | 43.8 ± 2.1 | 45.7 ± 1.9 | 42.4 ± 1.7 | 42.8 ± 1.6 |
| Median | 45.0 | 45.4 | 41.6 | 46.7 | 43.3 | 43.6 |
| Min, Max | 37.6, 46.9 | 40.7, 48.2 | 39.5, 51.0 | 39.4, 51.0 | 37.6, 45.5 | 38.4, 45.7 |

### PHARMACOKINETIC-PHARMACODYNAMIC RELATIONSHIP

Plasma Pharmacokinetic-Pharmacodynamic Relationship

The relationship between plasma concentrations of bupivacaine and sensory testing was examined for the unilateral 15 mL 40K EDLA 2.5% treatment in Part 1. For pin-prick testing, analgesia (score≦1.0) was first observed at 3 hours post-injection and was last observed at 96 hours post-injection. Plasma bupivacaine concentrations following unilateral 15 mL 40K EDLA 2.5% treatment were decreasing between the 1 and 6 hour observations, and therefore did not correlate with the onset of analgesia. Similarly, somesthetic test results were consistently 0 (0=No, no change in temperature was perceived) at the 1, 3, and 6 hour observations while plasma bupivacaine concentrations were decreasing. Warmth detection threshold appeared to be near maximally increased from baseline across all post-injection time points, and did not change with changing plasma bupivacaine concentrations. Heat pain detection thresholds did decrease slightly at the 3 and 6 hour observations, but also decreased at the 24 hour time point and later while plasma bupivacaine concentrations were rising. Therefore, it appears that local sensory testing is unrelated to the plasma concentration of bupivacaine following administration of 15 mL 40K EDLA 2.5%.

The relationship between plasma concentrations of bupivacaine and sensory testing was also examined for the bilateral AB 0.5% treatment in Part 1. Plasma bupivacaine concentrations following bilateral AB 0.5% treatment rapidly increased by 30 minutes post-injection and then gradually decreased through 96 hours post-injection. Results across all sensory tests demonstrated a roughly similar pattern that mirrored the plasma bupivacaine concentrations, with a maximal or nearly maximal effect at 30 minutes post-injection and a gradual decline in the effect through 96 hours post-injection.

Tissue Pharmacokinetic-Pharmacodynamic Relationship

The relationship between tissue concentrations of bupivacaine and sensory testing was examined for each of the treatments in Part 2. Across 40K EDLA treatments in general, bupivacaine tissue concentrations increased with time to a maximum value at the 11 hour 50 minute or 23 hour 50 minute time points and then gradually decreased with time. For pin-prick testing, the mean scores generally decreased over time to a minimum value that occurred between 6 and 48 hours before increasing again. For somesthetic testing, the mean scores decreased to a minimum value prior to 6 hours and generally remained at near minimum through the 96 hour time point. For WDT and HPDT testing, the maximal effect generally corresponded to the time window of the maximal bupivacaine concentration (12-24 hours post-injection). Therefore, it appears that for pin-prick, WDT, and HPDT testing that tissue bupivacaine concentration following 40K EDLA treatment roughly corresponds temporally to sensory effect, although future studies would be necessary to demonstrate this conclusively. Across AB treatments in general, bupivacaine tissue concentrations were maximal at 1 hour post-injection and then gradually decreased through 96 hours post-injection. Results across all sensory tests demonstrated a roughly similar pattern to tissue bupivacaine concentrations, with a maximal or nearly maximal effect at 30 minutes post-injection and a gradual decrease in the effect through 96 hours post-injection.

Changes in Local Blood Flow Over Time

In order to evaluate the effect of different volumes and concentrations of 40K EDLA on local blood flow assessed by LASER Doppler, descriptive statistics on the percent change in blood flow velocity were calculated at each time point for each site at which local blood flow was assessed for each treatment in Parts 1 and 2. For Part 1, Tables J-14(A) and J-14(B) present the percent change in local blood flow velocity over time for 40K EDLA and AB, respectively. For Part 2, Tables J-14(C) and J-14(D) present the percent change in local blood flow velocity over time for 40K EDLA and AB, respectively.

TABLE J-14(A)
Percent Change in Blood Flow Velocity Over
Time for Part 1: 40K EDLA
Evaluable for Efficacy Population

| | | |
|---|---|---|
| 15 mL 40K | 15 mL 40K | 15 mL 40K |
| EDLA 2.5% | EDLA 2.5% | EDLA 2.5% |
| unilateral | right | left |

Baseline

| | | | |
|---|---|---|---|
| N | 4 | 3 | 3 |
| Mean | 0 | 0 | 0 |
| ±SEM | 0 | 0 | 0 |
| Median | 0 | 0 | 0 |
| Min, Max | 0, 0 | 0, 0 | 0, 0 |
| 30 | | | |
| minutes | | | |
| n | 4 | 3 | 3 |
| Mean | 222.8 | 234.7 | 146.7 |
| ±SEM | 55.4 | 157.4 | 61.3 |
| Median | 259.0 | 105.0 | 107.0 |
| Min, Max | 64.0, 309.0 | 51.0, 548.0 | 66.0, 267.0 |
| 1 hour | | | |
| n | 4 | 3 | 3 |
| Mean | 60.3 | −1.7 | 46.7 |
| ±SEM | 39.0 | 25.2 | 61.3 |
| Median | 37.0 | −15.0 | −7.0 |
| Min, Max | 0.0, 167.0 | −37.0, 47.0 | −22.0, 169.0 |
| 3 hours | | | |
| n | 3 | 3 | 3 |
| Mean | −93.7 | −48.0 | −66.7 |
| ±SEM | 48.0 | 29.7 | 41.7 |
| Median | −99.0 | −47.0 | −60.0 |
| Min, Max | −174.0, −8.0 | −100.0, 3.0 | −142.0, 2.0 |
| 6 hours | | | |
| n | 4 | 3 | 3 |
| Mean | −11.3 | −45.0 | −29.7 |
| ±SEM | 7.4 | 26.0 | 12.9 |
| Median | −9.5 | −45.0 | −41.0 |
| Min, Max | −31.0, 5.0 | −90.0, 0.0 | −44.0, −4.0 |
| 12 hours | | | |
| n | 3 | 2 | 2 |
| Mean | 9.7 | −32.0 | −3.5 |
| ±SEM | 19.6 | 3.0 | 52.5 |
| Median | 16.0 | −32.0 | −3.5 |
| Min, Max | −27.0, 40.0 | −35.0, −29.0 | −56.0, 49.0 |
| 24 hours | | | |
| n | 4 | 3 | 3 |
| Mean | −8.8 | −17.7 | −18.7 |
| ±SEM | 31.2 | 22.7 | 18.0 |
| Median | −2.5 | 5.0 | −7.0 |
| Min, Max | −86.0, 56.0 | −63.0, 5.0 | −54.0, 5.0 |
| 48 hours | | | |
| n | 4 | 3 | 3 |
| Mean | −28.0 | 24.0 | 34.0 |
| ±SEM | 15.2 | 40.2 | 36.0 |
| Median | −35.5 | 2.0 | 17.0 |
| Min, Max | −56.0, 15.0 | −32.0, 102.0 | −18.0, 103.0 |
| 72 hours | | | |
| n | 4 | 2 | 2 |
| Mean | 26.8 | −6.0 | 39.5 |
| ±SEM | 18.7 | 6.0 | 40.5 |
| Median | 26.0 | −6.0 | 39.5 |
| Min, Max | −7.0, 62.0 | −12.0, 0.0 | −1.0, 80.0 |
| 96 hours | | | |
| n | 4 | 2 | 2 |
| Mean | 26.0 | 0 | 18.5 |
| ±SEM | 24.6 | 2.0 | 4.5 |
| Median | 24.0 | 0 | 18.5 |
| Min, Max | −29.0, 85.0 | −2.0, 2.0 | 14.0, 23.0 |

TABLE J-14(B)
Percent Change in Blood Flow Velocity Over Time for Part 1: AB
Evaluable for Efficacy Population

| | |
|---|---|
| 15 mL AB 0.5% | 15 mL AB 0.5% |
| right | left |

Baseline

| | | |
|---|---|---|
| n | 4 | 4 |
| Mean | 0 | 0 |
| ±SEM | 0 | 0 |
| Median | 0 | 0 |
| Min, Max | 0, 0 | 0, 0 |
| 30 minutes | | |
| n | 3 | 3 |
| Mean | 141.7 | 141.3 |
| ±SEM | 130.2 | 53.5 |
| Median | 67.0 | 135.0 |
| Min, Max | −37.0, 395.0 | 52.0, 237.0 |
| 1 hour | | |
| n | 3 | 3 |
| Mean | 68.0 | 97.3 |
| ±SEM | 109.2 | 54.9 |
| Median | −12.0 | 77.0 |
| Min, Max | −68.0, 284.0 | 14.0, 201.0 |
| 3 hours | | |
| n | 3 | 3 |
| Mean | 39.7 | 24.3 |
| ±SEM | 71.7 | 11.8 |
| Median | 16.0 | 13.0 |
| Min, Max | −71.0, 174.0 | 12.0, 48.0 |
| 6 hours | | |
| n | 4 | 4 |
| Mean | −38.8 | −13.0 |
| ±SEM | 23.6 | 19.0 |
| Median | −53.0 | −24.5 |
| Min, Max | −78.0, 29.0 | −45.0, 42.0 |
| 12 hours | | |
| n | 3 | 3 |
| Mean | −14.0 | 0.3 |
| ±SEM | 37.3 | 29.9 |
| Median | −11.0 | −15.0 |
| Min, Max | −80.0, 49.0 | −42.0, 58.0 |
| 24 hours | | |
| n | 4 | 4 |
| Mean | −1.3 | 111.3 |
| ±SEM | 18.1 | 111.9 |
| Median | −3.0 | 7.0 |
| Min, Max | −42.0, 43.0 | −15.0, 446.0 |
| 48 hours | | |
| n | 4 | 4 |
| Mean | 3.8 | 15.8 |
| ±SEM | 24.6 | 12.3 |
| Median | −16.0 | 17.0 |
| Min, Max | −29.0, 76.0 | −15.0, 44.0 |
| 72 hours | | |
| n | 4 | 4 |
| Mean | 0.8 | 2.5 |
| ±SEM | 35.6 | 24.9 |
| Median | −27.0 | −21.0 |
| Min, Max | −49.0, 106.0 | −25.0, 77.0 |
| 96 hours | | |
| n | 4 | 4 |
| Mean | −22.8 | −2.0 |
| ±SEM | 26.8 | 15.2 |
| Median | −28.5 | 4.5 |
| Min, Max | −78.0, 44.0 | −40.0, 23.0 |

TABLE J-14(C)
Percent Change in Blood Flow Velocity Over
Time for Part 2: 40K EDLA
Evaluable for Efficacy Population

| | 7.5 mL | 15 mL | 15 mL | 15 mL |
|---|---|---|---|---|
| | 40K | 40K | 40K | 40K |
| | EDLA | EDLA | EDLA | EDLA |
| | 2.5% | 2.5% | 1.25% | 2.5% |
| | right | Left | right | left |
| Baseline | | | | |
| n | 6 | 6 | 5 | 5 |
| Mean | 0 | 0 | 0 | 0 |
| ±SEM | 0 | 0 | 0 | 0 |
| Median | 0 | 0 | 0 | 0 |
| Min, Max | 0, 0 | 0, 0 | 0, 0 | 0, 0 |
| 30 | | | | |
| minutes | | | | |
| n | 5 | 6 | 5 | 5 |
| Mean | 110.4 | 142 | 86.8 | 85.2 |
| ±SEM | 81.4 | 65.8 | 46.9 | 73.2 |
| Median | 73.0 | 114.0 | 112.0 | 75.0 |
| Min, Max | −46.0, | −20.0, | −58.0, | −119.0, |
| | 396.0 | 436.0 | 213.0 | 332.0 |
| 1 hour | | | | |
| n | 6 | 6 | 5 | 5 |
| Mean | −52.5 | −26.7 | 65.0 | 18.0 |
| ±SEM | 25.0 | 45.4 | 41.9 | 36.5 |
| Median | −35.5 | −42.5 | 73.0 | 7.0 |
| Min, Max | −145.0, | −178.0, | −52.0, | −53.0, |
| | 23.0 | 121.0 | 200.0 | 154.0 |
| 3 hours | | | | |
| N | 5 | 6 | 5 | 5 |
| Mean | 4.8 | 26.7 | −23.8 | −48.2 |
| ±SEM | 13.7 | 39.5 | 35.5 | 21.5 |
| Median | −4.0 | 3.5 | −29.0 | −31.0 |
| Min, Max | −26.0, | −51.0, | −107.0, | −107.0, |
| | 53.0 | 215.0 | 98.0 | 13.0 |
| 6 hours | | | | |
| n | 6 | 6 | 5 | 5 |
| Mean | −42.8 | −21.7 | −3.0 | −14.6 |
| ±SEM | 30.1 | 10.1 | 15.6 | 16.0 |
| Median | −25.0 | −28.0 | 19.0 | 4.0 |
| Min, Max | −185.0, | −42.0, | −48.0, | −53.0, |
| | 17.0 | 24.0 | 27.0 | 22.0 |
| 12 hours | | | | |
| N | 6 | 6 | 5 | 5 |
| Mean | −11.3 | −5.5 | −6.0 | −9.8 |
| ±SEM | 13.0 | 16.8 | 17.2 | 34.4 |
| Median | −17.5 | −9.5 | 0 | −29.0 |
| Min, Max | −44.0, | −50.0, | −62.0, | −79.0, |
| | 44.0 | 68.0 | 45.0 | 121.0 |
| 24 hours | | | | |
| n | 6 | 6 | 5 | 5 |
| Mean | −18.3 | −25.7 | −36.2 | −6.0 |
| ±SEM | 17.1 | 9.6 | 25.3 | 22.4 |
| Median | −1.5 | −21.5 | −44.0 | −22.0 |
| Min, Max | −77.0, | −69.0, | −120.0, | −52.0, |
| | 22.0 | 0.0 | 30.0 | 78.0 |
| 48 hours | | | | |
| n | 6 | 6 | 5 | 5 |
| Mean | −13.8 | −23.0 | −2.0 | 46.8 |
| ±SEM | 17.1 | 17.5 | 46.8 | 37.1 |
| Median | −6.5 | −9.0 | 3.0 | 24.0 |
| Min, Max | −83.0, | −100.0, | −171.0, | −14.0, |
| | 30.0 | 20.0 | 107.0 | 190.0 |
| 72 hours | | | | |
| N | 6 | 6 | 5 | 5 |
| Mean | −31.2 | 7.0 | 93.6 | 78.4 |
| +SEM | 13.9 | 36.0 | 77.5 | 31.5 |
| Median | −37.5 | −21.5 | 10.0 | 69.0 |
| Min, Max | −78.0, | −53.0, | −17.0, | −3.0, |
| | 22.0 | 183.0 | 398.0 | 182.0 |
| 96 hours | | | | |
| n | 6 | 6 | 5 | 5 |
| Mean | −26.0 | −3.8 | −28.2 | −8.0 |
| ±SEM | 10.8 | 21.9 | 15.6 | 26.4 |
| Median | −21.0 | −20.5 | −41.0 | −26.0 |
| Min, Max | −65.0, | −48.0, | −71.0, | −70.0, |
| | −2.0 | 97.0 | 14.0 | 83.0 |

TABLE J-14(D)
Percent Change in Blood Flow Velocity Over Time for Part 2: AB
Evaluable for Efficacy Population

| | 15 mL AB 0.25% | 15 mL AB 0.25% |
|---|---|---|
| | right | left |
| Baseline | | |
| n | 4 | 4 |
| Mean | 0 | 0 |
| ±SEM | 0 | 0 |
| Median | 0 | 0 |
| Min, Max | 0, 0 | 0, 0 |
| 30 | | |
| minutes | | |
| n | 4 | 4 |
| Mean | 90.8 | 17.0 |
| ±SEM | 85.8 | 32.6 |
| Median | 19.0 | 0 |
| Min, Max | −21.0, 346.0 | −39.0, 107.0 |
| 1 hour | | |
| n | 4 | 4 |
| Mean | 38.3 | −17 |
| ±SEM | 38.3 | 10.6 |
| Median | 13.0 | −14.0 |
| Min, Max | −22.0, 149.0 | −44.0, 4.0 |
| 3 hours | | |
| N | 4 | 4 |
| Mean | −33.0 | −37.8 |
| ±SEM | 16.4 | 10.5 |
| Median | −18.0 | −31.5 |
| Min, Max | −82.0, −14.0 | −68.0, −20.0 |
| 6 hours | | |
| n | 4 | 4 |
| Mean | −78.0 | −97.0 |
| ±SEM | 47.0 | 54.7 |
| Median | −54.5 | −56.0 |
| Min, Max | −202.0, −1.0 | −256.0, −20.0 |
| 12 hours | | |
| N | 4 | 4 |
| Mean | −47.5 | −91.8 |
| ±SEM | 39.3 | 26.2 |
| Median | −22.0 | −77.0 |
| Min, Max | −162.0, 16.0 | −164.0, −49.0 |
| 24 hours | | |
| n | 4 | 4 |
| Mean | 35.3 | 24.5 |
| ±SEM | 33.5 | 22.3 |
| Median | 14.5 | 24.5 |
| Min, Max | −20.0, 132.0 | −30.0, 79.0 |
| 48 hours | | |
| n | 4 | 4 |
| Mean | 1.5 | −18.5 |
| ±SEM | 14.4 | 7.6 |
| Median | 5.0 | −17.5 |
| Min, Max | −36.0, −32.0 | −37.0, −2.0 |
| 72 hours | | |
| N | 4 | 4 |
| Mean | −10.8 | −29.3 |
| ±SEM | 28.6 | 23.0 |
| Median | −11.5 | −34.5 |
| Min, Max | −77.0, 57.0 | −70.0, 22.0 |
| 96 hours | | |
| n | 4 | 4 |
| Mean | −5.0 | −26.0 |
| ±SEM | 16.6 | 11.1 |
| Median | −3.0 | −26.0 |
| Min, Max | −38.0, 24.0 | −48.0, −4.0 |

Blood flow velocity increased to approximately 85-235% of baseline across 40K EDLA groups at 30 minutes post-injection. An increase in blood flow velocity was also seen with AB at 30 minutes (17-142% of baseline). Following the 30-minute time point for both 40K EDLA and AB, blood flow velocity changed inconsistently from baseline. Differences in blood flow velocity did not appear to be related to the concentration or volume of 40K EDLA administered.

### Efficacy and/or Pharmacology Discussion and Conclusions

Treatment with bilateral AB 0.5% resulted in a rapid distribution of bupivacaine (tmax=0.81 h) into the systemic circulation compared to either unilateral (59.4 h) or bilateral (58.9 h) 40K EDLA 2.5% treatment. The peak and total exposure to bupivacaine in the plasma was approximately double for the bilateral 40K EDLA 2.5% treatment versus the unilateral 40K EDLA 2.5% treatment; the peak and total exposure to dexamethasone demonstrated a similar dose-proportional relationship. Across all treatments, the plasma Cmax for bupivacaine as 40K EDLA was consistently lower than that for AB, despite a larger total dose of bupivacaine in 40K EDLA-treated subjects.

The microdialysis methodology measured a similar peak and total tissue exposure to bupivacaine at two different sites treated with the same dose of 40K EDLA, and at two different sites treated with the same dose of AB; this was also true for dexamethasone. Although the peak and total tissue exposure to bupivacaine as 40K EDLA increased with increasing dose, a doubling of the delivered dose resulted in less than a doubling of peak and total exposure, regardless of whether the dose was increased by increasing volume or concentration; this was also true for dexamethasone.

Local sensory testing appeared to be unrelated to plasma bupivacaine concentration for the unilateral 15 mL 40K EDLA 2.5% treatment; however, local sensory test results roughly correlated with plasma bupivacaine concentration for the bilateral 15 mL AB 0.5% treatment. Local sensory test results did appear to roughly correlate with local tissue concentrations of bupivacaine delivered either as 40K EDLA or AB.

Local blood flow velocity increased at 30 minutes post-injection with both 40K EDLA and AB treatment, but was changed inconsistently by treatment at subsequent time points.

MRI testing under the current conditions was concluded to have limited utility in future studies of 40K EDLA.

### Safety Evaluation

Safety analyses were performed on all subjects who received study medication. Overall, a total of 28 subjects were included. Table J-15(A) presents the incidence of local adverse events by treatment. Table J-15(B) presents the incidence of systemic adverse events by treatment.

TABLE J-15(A)
Incidence of Local Adverse Events
Safety Population, N = 52 Unique Injection Sites, 28 Subjects

| PART ONE | | | | | PART TWO | | | | | |
|---|---|---|---|---|---|---|---|---|---|---|
| 15 mL | 15 mL | 15 mL | | | 7.5 mL | 15 mL | 15 mL | 15 mL | | |
| 40K | 40K | 40K | 15 mL | 15 mL | 40K | 40K | 40K | 40K | 15 mL | 15 mL |
| EDLA | EDLA | EDLA | AB | AB | EDLA | EDLA | EDLA | EDLA | AB | AB |
| 2.5% | 2.5% | 2.5% | 0.5% | 0.5% | 2.5% | 2.5% | 1.25% | 2.5% | 0.25% | 0.25% |
| left | right | left | right | left | right | left | right | left | right | left |
| calf | calf | calf | calf | calf | calf | calf | calf | calf | calf | calf |
| (N = 4) | (N = 4) | (N = 4) | (N = 4) | (N = 4) | (N = 6) | (N = 6) | (N = 6) | (N = 6) | (N = 4) | (N = 4) |

Number (%) of Unique Injection Sites

| | | | | | | | | | | | |
|---|---|---|---|---|---|---|---|---|---|---|---|
| Unique | 3 (75) | 3 (75) | 3 (75) | 4 (100) | 4 (100) | 5 (83) | 5 (83) | 6 (100) | 6 (100) | 4 (100) | 3 (75) |
| injection sites | | | | | | | | | | | |
| with at least 1 | | | | | | | | | | | |
| local AE | | | | | | | | | | | |

Number (%) of Unique Injection Sites

| | | | | | | | | | | | |
|---|---|---|---|---|---|---|---|---|---|---|---|
| Body System/ | | | | | | | | | | | |
| COSTART | | | | | | | | | | | |
| Term | | | | | | | | | | | |
| Body as a | 2 (50) | 2 (50) | 2 (50) | 0 | 0 | 2 (33) | 3 (50) | 4 (67) | 3 (50) | 2 (50) | 2 (50) |
| whole | | | | | | | | | | | |
| Injection site | 2 (50) | 2 (50) | 2 (50) | 0 | 0 | 2 (33) | 2 (33) | 4 (67) | 3 (50) | 1 (25) | 1 (25) |
| reaction | | | | | | | | | | | |
| Injection site | 0 | 1 (25) | 1 (25) | 0 | 0 | 0 | 0 | 0 | 0 | 0 | 0 |
| mass | | | | | | | | | | | |
| Injection site | 0 | 0 | 0 | 0 | 0 | 0 | 0 | 0 | 0 | 1 (25) | 1 (25) |
| hemorrhage | | | | | | | | | | | |
| Injection site | 0 | 0 | 0 | 0 | 0 | 0 | 1 (17) | 0 | 0 | 0 | 0 |
| pain | | | | | | | | | | | |
| Hematic & | 1 (25) | 1 (25) | 1 (25) | 4 (100) | 4 (100) | 4 (67) | 4 (67) | 4 (67) | 5 (83) | 1 (25) | 1 (25) |
| lymphatic | | | | | | | | | | | |
| Ecchymosis | 1 (25) | 1 (25) | 1 (25) | 4 (100) | 4 (100) | 4 (67) | 4 (67) | 4 (67) | 5 (83) | 1 (25) | 1 (25) |
| Metabolic & | 0 | 0 | 0 | 0 | 0 | 1 (17) | 1 (17) | 0 | 0 | 0 | 0 |
| Nutritional | | | | | | | | | | | |
| Peripheral | 0 | 0 | 0 | 0 | 0 | 1 (17) | 1 (17) | 0 | 0 | 0 | 0 |
| Edema | | | | | | | | | | | |
| Nervous | 0 | 2 (50) | 2 (50) | 0 | 0 | 0 | 1 (17) | 2 (33) | 2 (33) | 1 (25) | 0 |
| Hypesthesia | 0 | 2 (50) | 2 (50) | 0 | 0 | 0 | 1 (17) | 2 (33) | 2 (33) | 1 (25) | 0 |
| Skin & | 1 (25) | 0 | 0 | 0 | 0 | 1 (17) | 1 (17) | 1 (17) | 2 (33) | 1 (25) | 1 (25) |
| Appendages | | | | | | | | | | | |
| Rash | 0 | 0 | 0 | 0 | 0 | 1 (17) | 1 (17) | 1 (17) | 2 (33) | 1 (25) | 0 |
| Vesiculobullous | 1 (25) | 0 | 0 | 0 | 0 | 0 | 1 (17) | 0 | 1 (17) | 0 | 0 |
| rash | | | | | | | | | | | |
| Pruritis | 0 | 0 | 0 | 0 | 0 | 0 | 0 | 0 | 0 | 1 (25) | 1 (25) |

Local adverse events occurred at 75-100% of unique injection sites for each treatment. The 3 most common local adverse events were ecchymosis, injection site reaction, and hypesthesia.

Ecchymosis occurred at 3/12 (25%) unique injection sites across the three 15 mL 40K EDLA 2.5% treatments in Part 1; it also occurred at 8/8 (100%) unique injection sites across the two 15 mL AB 0.5% treatments in Part 1. In Part 2, ecchymosis occurred at 4/6 (67%) unique injection sites for the 7.5 mL 40K EDLA 2.5% treatment and at 4/6 (67%) unique injection sites for the 15 mL 40K EDLA 1.25% treatment. Ecchymosis occurred at 9/12 (75%) unique injection sites across the two 15 mL 40K EDLA 2.5% treatments. Ecchymosis occurred at 2/8 (25%) unique injection sites across the two 15 mL AB 0.25% treatments.

Injection site reaction occurred at 6/12 (50%) unique injection sites across the three 15 mL 40K EDLA 2.5% treatments in Part 1, but did not occur at sites injected with 15 mL AB 0.5% (0/8 sites). In Part 2, injection site reaction occurred at 2/6 (33%) injection sites for 7.5 mL 40K EDLA 2.5% and at 4/6 (67%) injection sites for 15 mL 1.25% 40K EDLA. Injection site reaction occurred at 5/12 (42%) unique injection sites across the two 15 mL 40K EDLA 2.5% treatments in Part 2. Injection site reaction occurred at 2/8 (25%) unique injection sites across the two 15 mL AB 0.25% treatments in Part 2.

In Part 1, hypesthesia occurred at 4/12 (33%) unique injection sites across the three 15 mL 40K EDLA 2.5% treatments; it did not occur with the 15 mL AB 0.5% treatments (0/8 sites). In Part 2, hypesthesia occurred at 3/12 (25%) unique injection sites across the two 15 mL 40K EDLA 2.5% treatments. Hypesthesia occurred at 2/6 (33%) injection sites for the 15 mL 40K EDLA 1.25% treatment, but did not occur for the 7.5 mL 40K EDLA 2.5% treatment. Hypesthesia occurred at ⅛ (13%) unique injection sites across the two 15 mL AB 0.25% treatments.

TABLE J-15(B)
Incidence of Systemic Adverse Events
Safety Population, N = 28 Subjects

| | | | | | |
|---|---|---|---|---|---|
| 15 mL | | | 7.5 mL 40K | | |
| 40K | 15 mL 40K EDLA | 15 mL AB 0.5% | EDLA 2.5% right | 15 mL 40K EDLA | 15 mLAB 0.25% |
| EDLA | 2.5% right calf | right calf | calf | 1.25% right calf | right calf |
| 2.5% | + | + | + | + | + |
| left | 15 mL 40K EDLA | 15 mL AB 0.5% | 15 mL 40K EDLA | 15 mL 40K EDLA | 15 mL AB 0.25% |
| calf | 2.5% left calf | left calf | 2.5% left calf | 2.5% left calf | left calf |
| (N = 4) | (N = 4) | (N = 4) | (N = 6) | (N = 6) | (N = 4) |

Number (%) of Subjects

| | | | | | | |
|---|---|---|---|---|---|---|
| Subjects with | 3 (75) | 1 (25) | 1 (25) | 1 (17) | 2 (33) | 1(25) |
| at least 1 | | | | | | |
| systemic AE | | | | | | |

Number (%) of Subjects

| | | | | | | |
|---|---|---|---|---|---|---|
| Body System/ | | | | | | |
| COSTART | | | | | | |
| Term | | | | | | |
| Body as a | 1 (25) | 1 (25) | 0 | 0 | 0 | 0 |
| Whole | | | | | | |
| Headache | 1 (25) | 0 | 0 | 0 | 0 | 0 |
| Back Pain | 0 | 1 (25) | 0 | 0 | 0 | 0 |
| Cardio- | 1 (25) | 0 | 0 | 0 | 0 | 0 |
| vascular | | | | | | |
| Migraine | 1 (25) | 0 | 0 | 0 | 0 | 0 |
| Nervous | 0 | 0 | 0 | 1 (17) | 1 (17) | 0 |
| Dizziness | 0 | 0 | 0 | 1 (17) | 1 (17) | 0 |
| Respiratory | 1 (25) | 0 | 1 (25) | 0 | 1 (17) | 1 (25) |
| Lung Disease | 0 | 0 | 1 (25) | 0 | 0 | 0 |
| Pharyngitis | 1 (25) | 0 | 0 | 0 | 1 (17) | 1 (25) |

The unilateral 15 mL 40K EDLA 2.5% treatment in Part 1 was associated with the highest incidence of systemic adverse events (¾ [75%] subjects). The incidence of subjects with at least one systemic adverse event ranged from 17-33% across the other treatments.

The most common systemic adverse events were pharyngitis and dizziness. Pharyngitis occurred in ¼ (25%) subjects that received unilateral 15 mL 40K EDLA 2.5%, in ⅙ (17%) subjects that received 15 mL 40K EDLA 1.25%+15 mL 40K EDLA 2.5%, and in ¼ (25%) subjects that received bilateral 15 mL AB 0.25%. Pharyngitis did not occur in subjects that received bilateral 15 mL 40K EDLA 2.5% (0/4 subjects), bilateral AB 0.5% (0/4 subjects), or 7.5 mL 40K EDLA 2.5%+15 mL 40K EDLA 2.5% (0/6 sites).

Dizziness occurred in ⅙ (17%) subjects for the 7.5 mL 40K EDLA 2.5%+15 mL 40K EDLA 2.5% treatment and ⅙ (17%) subjects for the 15 mL 40K EDLA 1.25%+15 mL 40K EDLA 2.5% treatment. Dizziness did not occur in subjects treated with either unilateral (0/4 subjects) or bilateral (0/4 subjects) 15 mL 40K EDLA 2.5%, nor did it occur in subjects treated with bilateral injections of either concentration of AB (0/8 subjects).

There were no deaths or serious adverse events. There were no adverse events that resulted in discontinuation.

Adverse event data were examined to evaluate the incidence of local subcutaneous tissue reactions (ie, injection site reaction, injection site edema, and injection site mass) and local neurological effects (ie, hypesthesia, hyperesthesia, paresthesia, and injection site pain).

Local subcutaneous tissue reactions reported included injection site reaction and injection site mass. Injection site reaction occurred at 11/24 (46%) injection sites treated with 15 mL 40K EDLA 2.5%, 0/8 injection sites treated with AB 0.5%, 2/6 (33%) injection sites treated with 7.5 mL 40K EDLA 2.5%, 4/6 (67%) injection sites treated with 15 mL 40K EDLA 1.25%, and 2/8 (25%) injection sites treated with 15 mL AB 0.25%. Injection site mass occurred at 2/24 (8%) injection sites treated with 15 mL 40K EDLA 2.5%, and did not occur with any other treatment.

Local neurological effects included hypesthesia and injection site pain. Hypesthesia occurred at 7/24 (29%) injection sites treated with 15 mL 40K EDLA 2.5%, 0/8 injection sites treated with 15 mL AB 0.5%, 0/6 injection sites treated with 7.5 mL 40K EDLA 2.5%, 2/6 (33%) injection sites treated with 15 mL 40K EDLA 1.25%, and ⅛ (13%) injection sites treated with 15 mL AB 0.25%. Injection site pain occurred only with 15 mL 40K EDLA 2.5% treatment (1/24 [4%] injection sites).

### Individual Subject Changes: Shifts from Baseline

The majority of the clinical laboratory values were normal at Screening and at 96 hours post-injection. Shifts in laboratory parameters from normal or high at baseline to low at endpoint and normal or low at baseline to high at endpoint is presented in Table J-16

TABLE J-16
Shifts from Baseline in Laboratory Parameters
Safety Population, N = 28 subjects

| | | | | | |
|---|---|---|---|---|---|
| | | | | 15 mL 40K | |
| 15 mL 40K | | | 7.5 mL 40K | EDLA 1.25% | |
| EDLA | 15mL 40K | 15 mL | EDLA 2.5% right + | right + 15 mL | 15 mL AB |
| 2.5% | EDLA 2.5% | AB 0.5% | 15 mL 40K | 40K EDLA | 0.25% |
| unilateral | bilateral | bilateral | EDLA 2.5% left | 2.5% left | bilateral |
| (n = 4) | (n = 4) | (n = 4) | (n = 6) | (n = 6) | (n = 4) |

n (%) subjects

Laboratory Value

| | | | | | | |
|---|---|---|---|---|---|---|
| Normal to Low | | | | | | |
| Hemoglobin | 0 | 1 (25) | 0 | 0 | 1 (17) | 0 |
| Hematocrit | 0 | 1 (25) | 0 | 1 (17) | 1 (17) | 0 |
| RBC | 0 | 0 | 0 | 0 | 1 (17) | 0 |
| Lymphocytes | 0 | 0 | 0 | 0 | 1 (17) | 0 |
| Sodium | 1 (25) | 0 | 0 | 0 | 0 | 0 |
| CO₂ | 0 | 0 | 1 (25) | 2 (33) | 1 (17) | 0 |
| Uric acid | 1 (25) | 0 | 0 | 2 (33) | 0 | 0 |
| AST (SGOT) | 0 | 0 | 1 (25) | 0 | 0 | 0 |
| Glucose | 0 | 0 | 0 | 1 (17) | 0 | 0 |
| Total Protein | 0 | 0 | 0 | 2 (33) | 0 | 0 |
| Calcium | 0 | 0 | 0 | 1 (17) | 0 | 0 |
| Alkaline | 0 | 0 | 0 | 0 | 1 (17) | 0 |
| Phosphatase | | | | | | |
| Normal to High | | | | | | |
| Eosinophils | 0 | 1 (25) | 0 | 0 | 0 | 1 (25) |
| Monocytes | 0 | 0 | 0 | 1 (17) | 1 (17) | 0 |
| Chloride | 1 (25) | 0 | 0 | 2 (33) | 1 (17) | 0 |
| Total protein | 1 (25) | 0 | 0 | 0 | 0 | 0 |
| BUN | 0 | 0 | 0 | 2 (33) | 0 | 0 |
| Sodium | 0 | 0 | 0 | 0 | 3 (50) | 0 |
| ALT (SGPT) | 0 | 0 | 0 | 0 | 2 (33) | 0 |
| Triglycerides | 0 | 0 | 0 | 1 (17) | 1 (17) | 1 (25) |
| Cholesterol | 0 | 0 | 0 | 1 (17) | 0 | 0 |

The shift analysis revealed no shift of clinical concern for hematology or clinical chemistry parameters. No subjects shifted from low to high or from high to low.

The most common normal to low shift occurred for CO₂. CO₂ shifted from normal to low for ¼ (25%) subjects receiving bilateral 15 mL AB 0.5%, 2/6 (33%) subjects receiving 7.5 mL 40K EDLA 2.5%+15 mL 40K EDLA 2.5%, and ¼ (25%) subjects receiving 15 mL 40K EDLA 1.25%+15 mL 40K EDLA 2.5%. CO₂ did not shift in subjects receiving unilateral 15 mL 40K EDLA 2.5% (0/4 subjects), bilateral 15 mL 40K EDLA 2.5% (0/4 subjects), or bilateral 15 mL AB 0.25% (0/4 subjects).

The most common normal to high shift occurred for chloride. Chloride shifted from normal to high for ¼ (25%) subjects receiving unilateral 15 mL 40K EDLA 2.5%, 2/6 (33%) subjects receiving 7.5 mL 40K EDLA 2.5%+15 ML 40K EDLA 2.5%, and ⅙ (17%) subjects receiving 15 mL 40K EDLA 1.25%+15 mL 40K EDLA 2.5%. Chloride did not shift in subjects receiving bilateral 15 mL 40K EDLA 2.5% (0/4 subjects), bilateral 15 mL AB 0.5% (0/4 subjects), or bilateral 15 mL AB 0.25% (0/4 subjects).

Special Analysis of Liver Function Tests

Liver function tests for SGOT or SGPT that were >3 times the upper limit of normal were considered to be of clinical importance. There were no subjects that had a liver function test that changed from normal to >3 times the upper limit of normal between screening and final visit. One subject had SGPT at both screening (136 U/L) and 96 hours post-injection (124 U/L) that was >3 times the upper limit of normal (35 U/L).

Clinically Notable Laboratory Abnormalities

Table J-17 lists clinically notable laboratory abnormalities by subject and parameter

TABLE J-17
Clinically Notable Laboratory Abnormalities
Safety Population (N = 28)

| | | Abnormal | | |
|---|---|---|---|---|
| Treatment Group | Subject | Test | Visit | Value^{a} |
| 15 ml AB 0.5% | 101 | Triglycerides | Screen | ND^{b} |
| bilateral | | | End of Study | 617 mg/dL |
| 15 ml 40K EDLA | 105 | Hematocrit | Screen | 41% |
| 2.5% bilateral | | | End of Study | 37% |
| 7.5 ml 40K EDLA | 200 | CO₂ | Screen | 29 meq/L |
| 2.5% right, 15 ml | | | End of Study | 16 meq/L |
| 40K EDLA 2.5% | | | | |
| left | | | | |
| 15 ml 40K EDLA | 204 | Hematocrit | Screen | 41% |
| 1.25% right, 15 ml | | | End of Study | 36% |
| 40K EDLA 2.5% | | | | |
| left | | | | |
| 7.5 ml 40K EDLA | 208 | Triglycerides | Screen | 85 mg/dL |
| 2.5% right, 15 ml | | | End of Study | 654 mg/dL |
| 40K EDLA 2.5% | | | | |
| left | | | | |

^{a}Clinically notable abnormality is bolded.
^{b}Test not run by laboratory.

One subject (treated with bilateral 15 mL AB 0.5%) had a clinically notably high triglycerides level at end of study, however, this subject's triglycerides level was not measured at screening. A second subject (treated with 7.5 mL 40K EDLA 2.5%+15 mL 40K EDLA 2.5%) also had a clinically notably high triglycerides level at end of study. A third subject (treated with bilateral 15 mL 40K EDLA 2.5%) and a fourth subject (treated with 15 mL 40K EDLA 1.25%+15 mL 40K EDLA 2.5%) had clinically notably low hematocrit at end of study. A fifth subject (treated with 7.5 mL 40K EDLA 2.5%+15 mL 40K EDLA 2.5%) had a clinically notably low CO₂ at end of study.

Summary Statistics Over Time: Vital Signs

Table J-18 presents summary statistics for systolic blood pressure, diastolic blood pressure, heart rate, respiratory rate, and temperature at Baseline and 96 h post-injection.

TABLE J-18
Mean (SEM) Vital Signs and Mean Change From Baseline to Final Visit
Safety Population (N = 28)

| | | 15 mL 40K | | 7.5 mL 40K | 15 mL 40K | |
|---|---|---|---|---|---|---|
| | | EDLA 2.5% | | EDLA 2.5% | EDLA 1.25% | 15 mL AB |
| | | right calf | 15 mL AB | right calf | right calf | 0.25% right |
| | | + | 0.5% right calf | + | + | calf |
| | 15 mL 40K | 15 mL 40K | + | 15 mL 40K | 15 mL 40K | + |
| | EDLA 2.5% | EDLA 2.5% | 15 mL AB | EDLA 2.5% | EDLA 2.5% | 15 mL AB |
| | left calf | left calf | 0.5% left calf | left calf | left calf | 0.25% left calf |
| | (N = 4) | (N = 4) | (N = 4) | (N = 6) | (N = 6) | (N = 4) |
| Systolic BP (mm Hg) | | | | | | |
| Baseline mean (± | 114.5 ± 9.1 | 126.5 ± 5.6 | 116.5 ± 9.5 | 132.2 ± 3.6 | 123.7 ± 4.0 | 142.8 ± 7.5 |
| SEM) | | | | | | |
| Change at end-of- | 5.5 ± 6.7 | 0.3 ± 6.0 | 3.8 ± 7.8 | −0.2 ± 2.9 | 0.8 ± 6.5 | −9.0 ± 7.5 |
| study, mean (± | | | | | | |
| SEM) | | | | | | |
| Range | −14.0, 16.0 | −13.0, 16.0 | −13.0, 24.0 | −12.0, 8.0 | −16.0, 27.0 | −26.0, 6.0 |
| Diastolic BP (mm Hg) | | | | | | |
| Baseline mean (± | 72.8 ± 7.4 | 76.5 ± 3.6 | 69.8 ± 4.3 | 76.8 ± 3.7 | 72.2 ± 4.2 | 85.3 ± 5.5 |
| SEM) | | | | | | |
| Change at end-of- | 3.8 ± 6.9 | 2.3 ± 4.4 | 5.8 ± 3.4 | 3.3 ± 4.0 | 1.2 ± 6.1 | −2.5 ± 4.1 |
| study, mean (± | | | | | | |
| SEM) | | | | | | |
| Range | −14.0, 16.0 | −5.0, 15.0 | −1.0, 15.0 | −7.0, 19.0 | −23.0, 18.0 | −13.0, 7.0 |
| Radial Pulse (beats/min) | | | | | | |
| Baseline, mean (± | 69.8 ± 5.6 | 66.5 ± 6.1 | 70.5 ± 3.2 | 66.8 ± 3.7 | 65.8 ± 1.7 | 68.0 ± 5.2 |
| SEM) | | | | | | |
| Change at end-of- | 1.5 ± 3.1 | 4.8 ± 2.9 | 15.8* ± 1.5 | 4.2 ± 3.2 | 12.5 ± 6.9 | 1.0 ± 5.9 |
| study, mean (± | | | | | | |
| SEM) | | | | | | |
| Range | −6.0, 9.0 | 0.0, 12.0 | 12.0, 19.0 | −4.0, 18.0 | −12.0, 36.0 | −16.0, 11.0 |
| Respiratory Rate (brths/min) | | | | | | |
| Baseline mean (± | 18.0 | 16.5 | 16.0 | 16.0 | 16.3 | 15.5 |
| SEM) | | | | | | |
| Change at end-of- | −1.5 ± 2.1 | −2.0 ± 1.4 | −0.5 ± 1.5 | 0.0 ± 1.8 | −1.0 ± 1.6 | −0.5 ± 1.0 |
| study, mean (± | | | | | | |
| SEM) | | | | | | |
| Range | −6.0, 2.0 | −6.0, 0.0 | −4.0, 2.0 | −6.0, 6.0 | −4.0, 6.0 | −2.0, 2.0 |
| Temperature (° C.) | | | | | | |
| Baseline mean (± | 36.8 | 37.0 | 37.1 | 36.4 | 36.3 | 36.2 |
| SEM) | | | | | | |
| Change at end-of- | −0.7* ± 0.1 | −0.7* ± 0.1 | −0.2 ± 0.3 | −0.2 ± 0.2 | 0.3 ± 0.3 | −0.7* ± 0.1 |
| study, mean (± | | | | | | |
| SEM) | | | | | | |
| Range | −1.1, −0.5 | −1.0, −0.4 | −0.7, 0.5 | −0.8, 0.9 | −0.5, 1.4 | −0.9, −0.3 |

*Mean change is statistically significant (p < 0.05).

The following changes from Screening to Final Visit (96 hours post-injection) were statistically significant p<0.05): a decrease in radial pulse for the 15 mL bilateral AB 0.5% treatment and a decrease in temperature for the unilateral 15 mL 40K EDLA 2.5% treatment, the bilateral 15 mL 40K EDLA 2.5% treatment, and the bilateral 15 mL AB 0.25% treatment. None of the mean changes in vital signs results from baseline to final visit were considered clinically meaningful.

Clinically Notable Vital Sign Abnormalities

Table J-19 lists clinically notable vital sign abnormalities by subject and parameter, along with all other values during the study for that vital sign and parameter and other relevant vital sign parameters at selected time points.

TABLE J-19
Clinically Notable Vital Sign Abnormalities
Safety Population (N = 28)

| | | | SBP | DBP | HR | RR |
|---|---|---|---|---|---|---|
| Treatment Group | Subject | Visit | (mmHg)^{a} | (mmHg)^{a} | (bpm)^{a} | (breaths/min)^{a} |
| 15 mL 40K | 213 | Screening | 112 | 64 | 60 | 16 |
| EDLA 1.25% + | | | | | | |
| 15 mL 40K | | | | | | |
| EDLA 2.5% | | | | | | |
| | | Injection day | 104 | 41^{b} | 48 | 16 |
| | | 24 h post-inj. | 119 | 53 | 69 | 16 |
| | | 48 h post-inj. | 107 | 39 | 55 | 14 |
| | | 72 h post-inj. | 103 | 44 | 55 | 16 |
| | | 96 h post-inj. | 111 | 69 | 61 | 14 |
| 15 mL 40K | 107 | Screening | 110 | 80 | 76 | 14 |
| EDLA 2.5% | | | | | | |
| bilateral | | | | | | |
| | | Injection day | 123 | 75 | 49^{c} | 16 |
| | | 24 h post-inj. | 122 | 78 | 69 | 12 |
| 15 mL AB 0.5% | 106 | Screening | 118 | 70 | 76 | 14 |
| bilateral | | | | | | |
| | | Injection day | 136 | 81 | 68 | 18 |
| | | 24 h post-inj. | 134 | 86 | 68 | 10 |
| | | 48 h post-inj. | 136 | 87 | 68 | 16 |
| 15 mL 40K | 104 | Screening | 116 | 70 | 88 | 16 |
| EDLA 2.5% left | | | | | | |
| | | 96 h post-inj. | 133 | 78 | 62 | 24 |

^{a}Clinically notable abnormality is bolded.
^{b}This value is the lowest of five clinically notable DBP values recorded on the day of injection.
^{c}Occurred at 1 hour post-injection.

Subject #213 had a diastolic blood pressure that decreased following drug injection. This subject's DBP was 65 prior to injection, 46 at 30 minutes post-injection, 43 at 1 hour post-injection, 41 at 3 hours post-injection, 47 at 6 hours post-injection, and 43 at 12 hours post-injection. This subject's DBP was also clinically notably decreased at 48 and 72 hours post-injection, but returned to normal range at 96 hours post-injection. None of this subject's other vital signs were clinically notably abnormal. Subject #107 had a decreased heart rate (49 bpm) one hour following injection. This subject's heart rate returned to normal range at 3 hours post-injection and remained in the normal range thereafter. This subject had no other clinically notable vital sign abnormalities.

Subject #106 had a clinically notably low respiratory rate of 10 breaths per minute at 24 hours post-injection. This subject had no other clinically notable vital sign abnormalities. There were 21 subjects that had post-screening values for respiratory rate that were on the border of the clinically notable range. Ten of these subjects had a respiratory rate value of 12 breaths per minute, and 11 had a respiratory rate value of 20 breaths per minute. Since these values were not consistently produced by any one treatment and were evenly distributed between the high and low end of the normal range, it is unlikely that they reflect an effect of study drug administration.

Physical Examination Findings and Medical History

Abnormal physical examination findings at end of study occurred in the majority (22/28) of subjects and usually involved the skin and extremities. Most appeared to be associated with a local reaction to the injection of study medication. Medical history findings were unremarkable.

Safety Discussion and Conclusions

There were no deaths or other serious or significant adverse events.

Local adverse events occurred at 75-100% of unique injection sites for each treatment. The 3 most common local adverse events were ecchymosis, injection site reaction, and hypesthesia. These events occurred with both 40K EDLA and AB treatment.

The unilateral 15 mL 40K EDLA 2.5% treatment was associated with the highest incidence of systemic adverse events (¾ [75%] subjects). The most common systemic adverse events were pharyngitis and dizziness. Pharyngitis occurred with both 40K EDLA and AB treatment, while dizziness occurred with 40K EDLA but not AB treatment.

Shift analyses from screening to final visit revealed no shift of clinical concern for laboratory parameters. None of the clinically notable laboratory or vital sign abnormalities was considered a serious or significant adverse event.

Medical History at screening and Physical Examination and ECG evaluations at end of study were unremarkable.

### Overall Conclusions for Microdialysis

In this infiltration model, the peak and total plasma exposure to bupivacaine delivered as 40K EDLA was approximately proportional to dose. The microdialysis method employed to examine local tissue concentrations of bupivacaine appears to be reliable. The local tissue concentrations of bupivacaine did appear to correlate with local sensory testing. Overall, subcutaneous infiltration of 40K EDLA was well-tolerated.

For Part 1 of the study:

Aqueous Bupivacaine

Aqueous bupivacaine distributes rapidly into systemic circulation

Peak exposure to bupivacaine in the tissue is e.g., approximately 40-fold greater than in the plasma

EDLA

Bupivacaine from EDLA distributes slowly into systemic circulation

Peak exposure to bupivacaine in the tissue is e.g., approximately 200-fold greater than in the plasma

Peak exposure to dexamethasone in the tissue is e.g., approximately 300-fold greater than in the plasma

For Part II of the study:

EDLA

Bupivacaine from EDLA distributes slowly into systemic circulation

Peak exposure to bupivacaine in the tissue from 1.25% EDLA, 15 ml (405 mg bupivacaine) is similar to that of 2.5% EDLA, 7.5 ml (405 mg bupivacaine)

Peak and total exposure to bupivacaine in the tissue increases with increasing dose. The increase in total exposure is less than proportional. However, the peak exposure increases proportionally.

There is no apparent effects of volume/concentration on peak or total exposure to bupivacaine and dexamethasone either locally or systemically

### Overall Safety Conclusions for In-vivo Studies

The generally recognized maximum recommended dose of aqueous bupivacaine for single administration is 225 mg when co-administered with epinephrine 1:200,000 (Marcaine® PI). The basis for establishing a maximum recommended dose for bupivacaine is to minimize the risk of central nervous system (CNS) and cardiovascular system toxicity. While there is no general agreement in the literature regarding the absolute toxic threshold of bupivacaine plasma concentrations, bupivacaine toxicity has been most commonly reported at concentrations in excess of 2-4 μg/mL Although the bupivacaine concentration is likely the key determinant of systemic toxicity, the rapidity with which a particular blood level is achieved may also influence the toxicity profile of local anesthetic agents.

Aqueous bupivacaine doses well in excess of the maximum labeled single dose of 225 mg are routinely administered over a period of days via indwelling catheters for post-operative pain purposes. When properly placed, these catheters usually provide safe and effective analgesia. EDLA is a polymer-based depot formulation that mimics a conventional continuous infusion of local anesthetic by releasing bupivacaine to the desired neural elements by slow, local diffusion from intact microspheres at the injection site.

The plasma concentration of local anesthetic drugs varies considerably as a function of the site of injection. In the intercostal nerve study, EDLA and IDLA were injected in the vicinity of the intercostal nerves. This compartment is known to be more vascular than that used in microdialysis study (infiltration in the calf) and is generally accepted as the compartment associated with the greatest Cₘₐₓ and earliest Tₘₐₓ when aqueous local anesthetics are administered. For example, intercostal nerve block has been shown to produce more than three times the maximum plasma concentration of a local anesthetic than was seen after local infiltration.

The highest plasma bupivacaine Cₘₐₓ for an EDLA-treated subject via infiltration (540 mg bupivacaine in the calf) was 0.493 μg/mL (Tₘₐₓ=72 hours). In a similar manner, observed plasma bupivacaine concentrations were examined for intercostal administration, known to involve more rapid uptake of a local anesthetic into the systemic circulation. In this case, the greatest observed plasma bupivacaine Cₘₐₓ for an EDLA-treated subject in this model (216 mg bupivacaine) was 0.323 μg/mL (Tₘₐₓ=24 hours). In the same study, the greatest observed plasma bupivacaine Cₘₐₓ in an IDLA-treated subject (108 mg) was 0.259 μg/mL (Tₘₐₓ=24 hours). Following the administration of EDLA and IDLA in close proximity to the superficial radial nerve (27 mg bupivacaine in each case), the greatest observed plasma bupivacaine Cₘₐₓ for an EDLA-treated subject in this model was 0.262 μg/mL (Tₘₐₓ=72 hours) while that of an IDLA-treated subject reached 0.151 μg/mL (Tₘₐₓ=24 hours). In all of these studies, the Cₘₐₓ of bupivacaine remained well below the threshold for toxicity.

1. A method for providing local analgesia, local anesthesia or nerve blockade in a human, comprising administering at a site in a human a formulation comprising a plurality of controlled release microspheres comprising bupivacaine free base and a biocompatible, biodegradable polymer comprising a 65:35 DL copolymer of lactic and glycolic acid having free carboxylic acid end groups, said copolymer having a molecular weight of about 40 kDa to about 120 kDa, said microspheres comprising from about 60% to about 85% bupivacaine free base, by weight, said microspheres being contained in a pharmaceutically acceptable medium for parenteral administration, said formulation having a concentration of bupivacaine free base from about 2.25 mg/ml to about 36.0 mg/ml and the formulation including a total amount of bupivacaine free base from about 45 mg to about 360 mg prior to administration, such that said formulation provides local analgesia, local anesthesia or nerve blockade at the site of administration less than about 2 hours after first administration, and a duration of local analgesia, local anesthesia or nerve blockade which lasts for at least about 1 day after first administration.

2. The method of claim 1, wherein said formulation further comprises an augmenting agent in an amount effective to prolong the effect of the local anesthetic for a time period greater than that obtained via administration of said formulation without said augmenting agent, such that the duration of local analgesia, local anesthesia or nerve blockade lasts for at least about 2 days after first administration.

3. The method of claim 1, wherein the duration of local analgesia is from about 2 to about 4 days after first administration.

4. The method of claim 1, wherein the duration of local analgesia is from about 4 to about 7 days after first administration.

5. The method of claim 1, wherein the level of local anesthetic at the site of administration is at least 150 times the level of local anesthetic in the systemic blood plasma.

6. The method of claim 1, wherein the formulation further comprises a concentration of dexamethasone from about 2.5 mcg/ml to about 10.0 mcg/ml.

7. The method of claim 1, wherein the microspheres further comprise 0.04% dexamethasone by weight.

8. The method of claim 1, wherein the microspheres comprise about 72% bupivacaine base, by weight.

9. The method of claim 1, wherein the microspheres further comprise a polymer selected the group consisting of polyanhydrides, polyesters, polyorthoesters, proteins, and polysaccharides.

10. The method of claim 1, wherein the formulation provides an in-vitro dissolution of the local anesthetic from the biocompatible, biodegradable carrier under in-vitro conditions specified by the USP II Paddle Method, 100 RPM, 37 degrees Celsius, pH 3.0 in 900 ml of 10 mM sodium phosphate buffer, as follows:

| TIME (Hours) | Percent Release |
|---|---|
| 0.25 | about 2 to about 32 |
| 0.5 | about 3 to about 60 |
| 1 | about 6 to about 86 |
| 1.5 | about 9 to about 92 |
| 2 | about 12 to about 94 |
| 3 | about 17 to about 97 |
| 4 | about 23 to about 97 |

11. The method of claim 1, wherein the formulation provides an in-vitro dissolution of the local anesthetic from the biocompatible, biodegradable carrier under in-vitro conditions specified by the USP II Paddle Method, 100 RPM, 37 degrees Celsius, pH 3.0 in 900 ml of 10 mM sodium phosphate buffer, as follows:

| TIME (Hours) | Percent Release |
|---|---|
| 1 | From about 13 to about 36 |
| 2 | From about 33 to about 65 |
| 4 | From about 53 to about 87 |
| 8 | From about 72 to about 95 |
| 12 | From about 81 to about 98 |
| 18 | From about 89 to about 100 |
| 24 | From about 94 to about 100 |

12. The method of claim 1, wherein the formulation is administered perineurially.

13. The method of claim 1, wherein the formulation is administered subcutaneously.

14. The method of claim 1, wherein the formulation is administered intramuscularly.

15. The method of claim 12, wherein the formulation provides an effect characterized by a mechanical pain detection threshold test in human patients in which the lowest number of the von Frey hair in which half of the stimulations produces a sensation of pain or unpleasantness about 16 to about 18 from about 2 to at least about 48 hours after administration, where the median baseline test is about 15.

16. The method of claim 12, wherein the formulation provides an effect characterized by a warm detection threshold test in which the median lowest increase in temperature from 32 C. perceived by human patients, occurs at a temperature as follows in degrees C.: about 40.5 to about 44.05 at 2 hours after administration; about 40.15 to about 44.85 at 4 hours after administration; about 40.15 to about 46.3 at 8 hours after administration; from about 41.7 to about 46.35 at 24 hours after administration; about 41.55 at 48 hours after administration; from about 40.4 to about 46.55 at 72 hours after administration; from about 41.1 to about 45.7 at 96 hours after administration; based on a median baseline test from about 39.9 to about 41.95.

17. The method of claim 12, wherein the formulation provides an effect characterized by perception of a temperature as painful, said temperature being at least 3° C. greater than the temperature that is perceived as painful prior to administration of the formulation, having an onset of at least about 1 hour and a duration of at least about 2 days.

18. The method of claim 12, wherein the formulation provides an effect characterized by a mechanical pain response test in which human patients characterized the pain on stimulating the injected area 5 times with von Frey hair No. 17 on a Verbal Rank Scale of 0-10 where 0=no pain and 10=pain as bad as the patient could imagine, as follows, based on a median result for patients tested: about 1 at 2 hours after administration; about 1 at 4 hours after administration; about 1 at 8 hours after administration; from about 0 to about 0.5 at 24 hours after administration; from about 0 to about 0.5 at 48 hours after administration; from about 0 to about 1 at 72 hours after administration; from about 0 to about 1 at 96 hours after administration; and about 1 at 144 hours after administration, based on a median baseline test result of about 2.

19. The method of claim 1, wherein the administration is intercostally.

20. The method of claim 19, wherein the formulation provides an effect characterized by a pin prick pain response test in which the degree of pain was assessed by administering pin pricks in an area innervated by the intercostal nerve and assessed by O, 1 or 2 wherein O means the subject did not feel any pinpricks, 1 means the subject felt 2 or 3 pinpricks as touch or pressure and 2 means the subject felt 2 or 3 pinpricks as sharp, as follows, based on a mean result for patients tested: from about 1 to about 2 at 1 hour after administration; from about 0.5 to about 1.5 at 2 hours after administration; from 0 to about 1 at 6 hours after administration; from about 0 to about 0.75 at 24 hours after administration.

21. The method of claim 19, wherein the formulation provides an effect characterized by a numbness response test in which human patients characterized the numbness on stimulating the site of injection on a Verbal Rank Scale of 0-10 where 0=not numb and 10=totally numb, as follows, based on a mean result for patients tested: about 0 to about 4 at 2 hours after administration; about 0 to about 3 at 6 hours after administration; about 0 to about 2 at 12 hours and from 0 to about 2 at 24 hours.

22. The method of claim 19, wherein the mean Cmax of bupivacaine does not exceed 250 ng/mL, when administered intercostally.

23. The method of claim 22, wherein the mean Cmax of bupivacaine is from about 10 to about 20 ng/mL, when administered intercostally.

24. The method of claim 1, wherein the administration is at a single nerve and the local analgesia is measured by a pin prick response test in which the degree of pain was assessed by administering pin pricks in an area innervated by the superficial peroneal nerve and assessed by O, 1 or 2 wherein O means the subject did not feel any pinpricks (anesthesia), 1 means the subject felt 2 or 3 pinpricks as touch or pressure or felt one as touch or pressure and 1 as sharp (analgesia) and 2 means the subject felt 2 or 3 pinpricks as sharp.

25. The method of claim 1, wherein the administration is at a single nerve and wherein the maximum plasma bupivacaine concentration is less than about 25 ng/mL.

26. The method of claim 1, wherein the administration is at a single nerve and provides a effect characterized by a numbness response test in which human patients characterized the numbness on stimulating the site of injection on a Verbal Rank Scale of 0-10 where 0=not numb and 10=totally numb, as follows, based on a mean result for patients tested: about 0 to about 5 at 1 hours after administration; about 0 to about 4 at 6 hours after administration; about 0 to about 3 at 12 hours and from 0 to about 3 at 24 hours.

27. The method of claim 24, wherein the single nerve is the superficial peroneal nerve.

28. The method of claim 1, wherein the administration is to the superficial radial nerve.

29. The method of claim 28, wherein the local analgesia is measured by a pin prick response test in which the degree of pain was assessed by administering pin pricks in an area innervated by the superficial radial nerve and assessed by O, 1 or 2 wherein O means the subject did not feel any pinpricks (anesthesia), 1 means the subject felt 2 or 3 pinpricks as touch or pressure or felt one as touch or pressure and 1 as sharp (analgesia) and 2 means the subject felt 2 or 3 pinpricks as sharp.

30. The method of claim 28, wherein the maximum plasma bupivacaine concentration is less than about 100 ng/mL.

31. The method of claim 28, wherein the formulation provides an effect characterized by a numbness response test in which human patients characterized the numbness on stimulating the site of injection on a Verbal Rank Scale of 0-10 where 0=not numb and 10=totally numb, as follows, based on a mean result for patients tested: about 0 to about 5 at 1 hours after administration; about 0 to about 4 at 6 hours after administration; about 0 to about 3 at 12 hours and from 0 to about 3 at 24 hours.

32. The method of claim 1, wherein the polymer has a viscosity from about 0.25 to about 0.42 dL/g.

33. A method for providing local analgesia, local anesthesia or nerve blockade in a human comprising administering at a site in a human a unit dose of microspheres comprising a biocompatible, biodegradable carrier and bupivacaine or a pharmaceutically acceptable salt thereof, effective to provide local analgesia, local anesthesia or nerve blockade at the site of administration in a human which occurs less than about 2 hours after first administration, and a duration of local analgesia, local anesthesia or nerve blockade which lasts for at least about 1 day after first administration, wherein the mean Cmax of bupivacaine measured by microdialysis in the tissue at the site is from about 35,000 ng/ml to below a toxic concentration at the site and wherein a level of local anesthetic at the site of administration is at least 150 times the level of said local anesthetic which is absorbed systemically into blood plasma.

34. The method of claim 33, wherein said microspheres further comprises an effective amount of dexamethasone or a pharmaceutically acceptable salt thereof to prolong the effect of the bupivacaine for a time period greater than that obtained via administration of said formulation without said augmenting agent such that a duration of local analgesia, anesthesia or nerve blockade lasts for at least about 2 days after first administration, wherein the mean Cmax of dexamethasone measured by microdialysis in the tissue at the site is from about 45 ng/ml to below a toxic concentration at the site and wherein a level of augmenting agent at the site of administration is at least 250 times the level of augmenting agent absorbed systemically into blood plasma.

35. A method for providing local analgesia, local anesthesia or nerve blockade in a human, comprising administering a unit dose of microspheres comprising a biocompatible, biodegradable carrier and bupivacaine or a pharmaceutically acceptable salt thereof, effective to provide local analgesia, local anesthesia or nerve blockade at a site of administration in a human which occurs less than about 2 hours after first administration, and a duration of local analgesia, local anesthesia or nerve blockade which lasts for at least about 1 day after first administration, wherein the mean Tmax of bupivacaine at the tissue at the site occurs at a time point from about 10 hours to about 45 hours after first administration.

36. The method of claim 35, wherein said microspheres further comprise an effective amount of dexamethasone or a pharmaceutically acceptable salt thereof to prolong the effect of the bupivacaine for a time period greater than that obtained via administration of said microspheres without said dexamethasone, such that a duration of local analgesia, anesthesia or nerve blockade lasts for at least about 2 days after first administration, wherein the mean Tmax of dexamathasone at the tissue at the site occurs at a time point from about 5 hours to about 40 hours after first administration.

37. The method of claim 33, wherein the mean AUCt of bupivacaine at 96 hours measured by microdialysis in the tissue at the site is from about 2,000,000 ng/ml*h to about 4,000,000 ng/ml*h.

38. The method of claim 37, wherein said microspheres further comprise an effective amount of dexamethasone or a pharmaceutically acceptable salt thereof to prolong the effect of the bupivacaine for a time period greater than that obtained via administration of said microspheres without said dexamethasone, such that a duration of local analgesia, anesthesia or nerve blockade lasts for at least about 2 days after first administration, wherein the mean AUCt of dexamethasone at 96 hours measured by microdialysis in the tissue at the site is from about 800 ng/ml*h to about 3,000 ng/ml*h.

39. The method of claim 33, wherein the mean Cmax of bupivacaine in the plasma is below about 250 ng/ml.

40. The method of claim 34, wherein the mean Cmax of dexamethasone in the plasma is below about 0.50 ng/ml.

41. The method of claim 35, wherein the mean Tmax of bupivicaine occurs at a time point from about 25 hours to about 50 hours after first administration.

42. The method of claim 35, wherein the mean Tmax of dexamethasone occurs at a time point from about 12 hours to about 30 hours after first administration.

43. The method of claim 33, wherein the mean AUCt of bupivacaine at 96 hours in the plasma is below about 12,000 ng/ml*h.

44. The method of claim 38, wherein the mean AUCt of dexamethasone at 96 hours in the plasma is below about 15 ng/ml*h.

45. The method of claim 33, wherein the formulation provides an effect characterized by a mean pin prick pain response test which is less than 1.0 at 3 hours after first administration.

46. The method of claim 33, wherein the formulation provides an effect characterized by a pin mean prick pain response test which is less than 1.0 at 24 hours after first administration.

47. The method of claim 33, wherein the formulation provides an effect characterized by a pin mean prick pain response test which is less than 1.0 at 48 hours after first administration.

48. The method of claim 33, wherein the formulation provides an effect characterized by a pin mean prick pain response test which is less than 1.0 at 72 hours after first administration.

49. The method of claim 33, wherein the formulation provides an effect characterized by a pin mean prick pain response test which is less than 1.0 at 96 hours after first administration.

50. The method of claims 33, wherein the formulation provides an effect characterized by a mean somesthetic response test which is less than 0.6 at 3 hours after first administration.

51. The method of claim 33, wherein the formulation provides an effect characterized by a mean somesthetic response test which is less than 0.6 at 24 hours after first administration.

52. The method of claim 33, wherein the formulation provides an effect characterized by a mean somesthetic response test which is less than 0.6 at 48 hours after first administration.

53. The method of claim 33, wherein the formulation provides an effect characterized by a mean somesthetic response test which is less than 0.6 at 72 hours after first administration.

54. The method of claim 33, wherein the formulation provides an effect characterized by a mean somesthetic response test which is less than 0.6 at 96 hours after first administration.

55. The method of claims 33, wherein the formulation provides an effect characterized by a mean warmth detection threshold result which is at least 3 degrees C. over the baseline at 3 hours after first administration.

56. The method of claim 33, wherein the formulation provides an effect characterized by a mean warmth detection threshold result which is at least 3 degrees C. over the baseline at 24 hours after first administration.

57. The method of claim 33, wherein the formulation provides an effect characterized by a mean warmth detection threshold result which is at least 3 degrees C. over the baseline at 48 hours after first administration.

58. The method of claim 33, wherein the formulation provides an effect characterized by a mean warmth detection threshold result which is at least 3 degrees C. over the baseline at 72 hours after first administration.

59. The method of claim 33, wherein the formulation provides an effect characterized by a mean warmth detection threshold result which is at least 3 degrees C. over the baseline at 96 hours after first administration.

60. The method of claims 33, wherein the formulation provides an effect characterized by a mean warmth detection threshold result which is at least 3 degrees C. over the baseline at 3 hours after first administration.

61. The method of claim 33, wherein the formulation provides an effect characterized by a mean heat pain detection threshold result which is at least 3 degrees C. over the baseline at 24 hours after first administration.

62. The method of claim 33, wherein the formulation provides an effect characterized by a mean heat pain detection threshold result which is at least 3 degrees C. over the baseline at 48 hours after first administration.

63. The method of claim 33, wherein the formulation provides an effect characterized by a mean heat pain detection threshold result which is at least 3 degrees C. over the baseline at 72 hours after first administration.

64. The method of claim 33, wherein biocompatible, biodegradable carrier a copolymer of lactic acid and glycolic acid.

65. The method of claim 33, wherein the local anesthetic is bupivacaine free base.

66. The method of claim 33, wherein the local anesthetic is bupivacaine free base, the augmenting agent is dexamethasone, and the polymer is a copolymer of lactic and glycolic acid.

67. The method of claim 33, wherein the carrier comprises a polymer selected the group consisting of polyanhydrides, polyesters, copolymers of lactic acid and glycolic acid, polyorthoesters, proteins, and polysaccharides.

68. The method of claim 33, wherein the carrier is suspended in a pharmaceutically acceptable vehicle for injection.

69. A pharmaceutical formulation comprising a plurality of controlled release microspheres comprising bupivacaine free base and a biocompatible, biodegradable polymer comprising a 65:35 DL copolymer of lactic and glycolic acid having free carboxylic acid end groups, said copolymer having a molecular weight of about 40 kDa to about 120 kDa, said microspheres comprising from about 60% to about 85% bupivacaine free base, by weight, said microspheres being contained in a pharmaceutically acceptable medium for parenteral administration, such that the formulation has a concentration of bupivacaine free base from about 2.25 mg/ml to about 36.0 mg/ml and the formulation includes a total amount of bupivacaine free base from about 45 mg to about 360 mg prior to administration.

70. A unit dose pharmaceutical formulation suitable for parenteral administration to humans upon reconstitution with a pharmaceutically acceptable medium for parenteral administration comprising a plurality of controlled release microspheres comprising bupivacaine free base and a biocompatible, biodegradable polymer comprising a 65:35 DL copolymer of lactic and glycolic acid having free carboxylic acid end groups, said copolymer a molecular weight of about 40 kDa to about 120 kDa, said microspheres comprising from about 60% to about 85% bupivacaine free base, by weight, said microspheres including a total amount of bupivacaine free base from about 45 mg to about 360 mg prior to administration.

71. The formulation of claim 69, wherein the molecular weight of the polymer is about 40 kDa.

72. The formulation of claim 69, wherein the molecular weight of the polymer is about 120 kDa.

73. The formulation of claim 69, wherein the polymer has a viscosity from about 0.25 to about 0.42 dL/g.

74. The formulation of claim 69, wherein the microspheres are present in the medium in a concentration of about 6.25 mg/ml.

75. The formulation of claim 69, wherein the concentration of bupivacaine free base in said formulation is about 4.5 mg/ml.

76. The formulation of claim 69, wherein the microspheres are present in the medium in a concentration of about 12.5 mg/ml.

77. The formulation of claim 69, wherein the concentration of bupivacaine free base in said formulation is about 9.0 mg/ml.

78. The formulation of claim 69, wherein the microspheres are present in the medium in a concentration of about 25.0 mg/ml.

79. The formulation of claim 69, wherein the concentration of bupivacaine free base in said formulation is about 18.0 mg/ml.

80. The formulation of claim 69, further comprising dexamethasone in said formulation is about 2.5 mcg/ml to about 10.0 mcg/ml.

81. A method for providing local analgesia, local anesthesia or nerve blockade in a human, comprising administering at a site in a human a unit dose of microspheres comprising a biocompatible, biodegradable carrier and a local anesthetic, effective to provide local analgesia, local anesthesia or nerve blockade at the site of administration in a human which occurs less than about 2 hours after first administration, and a duration of local analgesia, local anesthesia or nerve blockade which lasts for at least about 1 day after first administration, wherein the mean Cmax of local anesthetic measured by microdialysis in the tissue at the site is from a Cmax therapeutically equivalent to 35,000 ng/ml bupivicaine to below a toxic concentration at the site.

82. The method of claim 81, wherein said formulation further comprises an augmenting agent in an amount effective to prolong the effect of the local anesthetic for a time period greater than that obtained via administration of said formulation without said augmenting agent such that a duration of local analgesia lasts for at least about 2 days after first administration, wherein the level of augmenting agent at the site of administration is at least 250 times the level of augmenting agent in the blood plasma.

83. The method of claim 81, wherein said microspheres further comprises an effective amount of a cortocosteroid to prolong the effect of the local anesthetic for a time period greater than that obtained via administration of said formulation without said augmenting agent such that a duration of local analgesia, anesthesia or nerve blockade lasts for at least about 2 days after first administration, wherein the mean Cmax of corticosteroid measured by microdialysis in the tissue at the site is from a Cmax therapeutically equivalent to 45 ng/ml dexamethasone to below a toxic concentration at the site.

84. A method of detecting the local concentration of a local anesthetic at a site of administration comprising administering a local anesthetic at a site of a human and measuring the concentration of said local anesthetic in the tissue of said site by microdialysis at one or more time intervals.

85. A method of detecting the local concentration of a corticosteroid at a site of administration comprising administering a corticosteroid at a site of a human and measuring the concentration of said local anesthetic in the tissue of said site by microdialysis at one or more time intervals.

86. A method for preparing a local anesthetic formulation suitable for obtaining local analgesia, local anesthesia or nerve blockade in a human, comprising preparing a plurality of controlled release microspheres comprising bupivacaine free base and a biocompatible, biodegradable polymer comprising a 65:35 DL copolymer of lactic and glycolic acid having free carboxylic acid end groups, said copolymer a molecular weight of about 40 kDa to about 120 kDa, said microspheres comprising from about 60% to about 85% bupivacaine free base, by weight, and containing said microspheres in a pharmaceutically acceptable medium for parenteral administration, such that the formulation has a concentration of bupivacaine free base from about 2.25 mg/ml to about 36.0 mg/ml and the formulation includes a total amount of bupivacaine free base from about 45 mg to about 360 mg prior to administration.

87. The method of claim 1, wherein said microspheres are microcapsules.

88. The formulation of claim 69, wherein said microspheres are microcapsules.

89. A method for providing local analgesia, local anesthesia or nerve blockade in a human, comprising administering at a site in a human a formulation comprising a plurality of controlled release microspheres comprising bupivacaine free base and a biocompatible, biodegradable polymer comprising a 65:35 DL copolymer of lactic and glycolic acid having free carboxylic acid end groups, said copolymer having a molecular weight of about 40 kDa to about 120 kDa, said microspheres comprising from about 60% to about 85% bupivacaine free base, by weight, said microspheres being contained in a pharmaceutically acceptable medium for administration, said formulation having a concentration of bupivacaine free base from about 2.25 mg/ml to about 36.0 mg/ml and the formulation including a total amount of bupivacaine free base from about 45 mg to about 360 mg prior to administration, such that said formulation provides local analgesia, local anesthesia or nerve blockade at the site of administration less than about 2 hours after first administration, and a duration of local analgesia, local anesthesia or nerve blockade which lasts for at least about 1 day after first administration.

90. A pharmaceutical formulation comprising a unit dose of microspheres comprising a biocompatible, biodegradable carrier and bupivacaine or a pharmaceutically acceptable salt thereof, effective to provide local analgesia, local anesthesia or nerve blockade at the site of administration in a human which occurs less than about 2 hours after first administration, and a duration of local analgesia, local anesthesia or nerve blockade which lasts for at least about 1 day after first administration, said formulation providing a mean Cmax of bupivacaine measured by microdialysis in the tissue at the site from about 35,000 ng/ml to below a toxic concentration at the site and a level of local anesthetic at the site of administration at least 150 times the level of said local anesthetic absorbed systemically into blood plasma.

91. The formulation of claim method of claim 90, wherein said microspheres further comprise an effective amount of dexamethasone or a pharmaceutically acceptable salt thereof to prolong the effect of the bupivacaine for a time period greater than that obtained via administration of said formulation without said augmenting agent such that a duration of local analgesia, anesthesia or nerve blockade lasts for at least about 2 days after first administration, said formulation providing a mean Cmax of dexamethasone measured by microdialysis in the tissue at the site from about 45 ng/ml to below a toxic concentration at the site and a level of augmenting agent at the site of administration at least 250 times the level of augmenting agent absorbed systemically into blood plasma.

92. A pharmaceutical formulation comprising a unit dose of microspheres comprising a biocompatible, biodegradable carrier and bupivacaine or a pharmaceutically acceptable salt thereof, effective to provide local analgesia, local anesthesia or nerve blockade at a site of administration in a human which occurs less than about 2 hours after first administration, and a duration of local analgesia, local anesthesia or nerve blockade which lasts for at least about 1 day after first administration, said formulation providing a mean Tmax of bupivacaine at the tissue at the site occurs which occurs at a time point from about 10 hours to about 45 hours after first administration.

93. The formulation of claim 92, wherein said microspheres further comprise an effective amount of dexamethasone or a pharmaceutically acceptable salt thereof to prolong the effect of the bupivacaine for a time period greater than that obtained via administration of said microspheres without said dexamethasone, such that a duration of local analgesia, anesthesia or nerve blockade lasts for at least about 2 days after first administration, said formulation providing a mean Tmax of dexamathasone at the tissue at the site which occurs at a time point from about 5 hours to about 40 hours after first administration.
